(19) United States
(12) Patent Application Publication
(10) Pub. No.: US 2024/0180880 A1
(43) Pub. Date: Jun. 6, 2024

(54) METHODS OF USING EHMT2 INHIBITORS IN TREATING OR PREVENTING BLOOD DISORDERS

(71) Applicant: Epizyme, Inc., Cambridge, MA (US)

(72) Inventors: Elayne PENEBRE, Auburndale, MA (US); Veronica GIBAJA, Dorchester, MA (US); Maria Alejandra RAIMONDI, Jamaica Plain, MA (US)

(21) Appl. No.: 18/454,612

(22) Filed: Aug. 23, 2023

Related U.S. Application Data
(63) Continuation of application No. 16/756,304, filed on Apr. 15, 2020, now abandoned.
(60) Provisional application No. 62/573,876, filed on Oct. 18, 2017.
(60) Provisional application No. 62/574,128, filed on Oct. 18, 2017.

(51) Int. Cl. A61K 31/4184; A61K 31/155; A61K 31/165; A61K 31/167; A61K 31/17; A61K 31/198; A61K 31/4045; A61K 31/407; A61K 31/4192; A61K 31/428; A61K 31/437; A61K 31/4406; A61K 31/454; A61K 31/4709; A61K 31/496; A61K 31/4965; A61K 31/4985; A61K 31/506; A61K 31/519; A61K 31/706; A61K 38/46; A61P 7/06; C07C 211/44; C07C 211/45; C07C 211/46; C07C 211/47; C07C 211/48

(52) U.S. Cl. CPC A61K 31/4184; A61K 31/155; A61K 31/165; A61K 31/167; A61K 31/17; A61K 31/198; A61K 31/4045; A61K 31/407; A61K 31/4192; A61K 31/428; A61K 31/437; A61K 31/4406; A61K 31/454; A61K 31/4709; A61K 31/496; A61K 31/4965; A61K 31/4985; A61K 31/506; A61K 31/519; A61K 31/706; A61K 38/465; A61P 7/06; C07C 211/44; C07C 211/45; C07C 211/46; C07C 211/47; C07C 211/48

(57) ABSTRACT

The present disclosure relates to a method of preventing or treating a blood disorder (e.g., sickle-cell disease) via administering an EHMT2 inhibitor compound disclosed herein or a pharmaceutical composition thereof to subjects in need thereof. The present disclosure also relates to the use of such compounds for research or other non-therapeutic purposes.

### RELATED APPLICATIONS

This application claims benefit of, and priority to, U.S. Application No. 62/573,876, filed on Oct. 18, 2017, and U.S. Application No. 62/574,128, filed on Oct. 18, 2017, the entire contents of each of which are incorporated herein by reference.

### BACKGROUND

Methylation of protein lysine residues is an important signaling mechanism in eukaryotic cells, and the methylation state of histone lysines encodes signals that are recognized by a multitude of proteins and protein complexes in the context of epigenetic gene regulation.

Histone methylation is catalyzed by histone methyltransferases (HMTs), and HMTs have been implicated in various human diseases. HMTs can play a role in either activating or repressing gene expression, and certain HMTs (e.g., euchromatic histone-lysine N-methyltransferase 2 or EHMT2, also called G9a) may methylate many nonhistone proteins, such as tumor suppressor proteins (see, e.g., Liu et al., Journal of Medicinal Chemistry 56:8931-8942, 2013 and Krivega et al., Blood 126(5):665-672, 2015).

### SUMMARY

In one aspect, the present disclosure provides methods of preventing or treating a blood disorder (e.g., sickle-cell disease), the method comprising administering to a subject in need thereof a therapeutically effective amount of an EHMT2 inhibitor. In some embodiments, the EHMT2 inhibitor is a compound disclosed herein. In some embodiments, the EHMT2 inhibitor is not 2-cyclohexyl-6-methoxy-N-[1-(1-methylethyl)-4-piperidinyl]-7-[3-(1-pyrrolidinyl)propoxy]-4-quinazolinamine; N-(1-isopropylpiperidin-4-yl)-6-methoxy-2-(4-methyl-1,4-diazepan-1-yl)-7-(3-(piperidin-1-yl)propoxy)quinazolin-4-amine; 2-(4,4-difluoropiperidin-1-yl)-N-(1-isopropylpiperidin-4-yl)-6-methoxy-7-(3-(pyrrolidin-1-yl)propoxy)quinazolin-4-amine; or 2-(4-isopropyl-1,4-diazepan-1-yl)-N-(1-isopropylpiperidin-4-yl)-6-methoxy-7-(3-(piperidin-1-yl)propoxy)quinazolin-4-amine. In some embodiments, the blood disorder is anemia. In some embodiments, the blood disorder is thalassemia. In some embodiments, the blood disorder is leukemia. In some embodiments, the blood disorder is lymphoma. In certain embodiments, the blood disorder is Acute lymphoblastic leukemia (ALL), Acute myeloid leukemia (AML) (e.g., acute promyelocytic leukemia, APL), Amyloidosis, Anemia, Aplastic anemia, Bone marrow failure syndromes, Chronic lymphocytic leukemia (CLL), Chronic myeloid leukemia (CML), Deep vein thrombosis (DVT), Diamond-Blackfan anemia, Dyskeratosis congenita (DKC), Eosinophilic disorder, Essential thrombocythemia, Fanconi anemia, Gaucher disease, Hemochromatosis, Hemolytic anemia, Hemophilia, Hereditary spherocytosis, Hodgkin's lymphoma, Idiopathic thrombocytopenic purpura (ITP), Inherited bone marrow failure syndromes, Iron-deficiency anemia, Langerhans cell histiocytosis, Large granular lymphocytic (LGL) leukemia, Leukemia, Leukopenia, Mastocytosis, Monoclonal gammopathy, Multiple myeloma, Myelodysplastic syndromes (MDS), Myelofibrosis, Myeloproliferative neoplasms (MPN), Non-Hodgkin's lymphoma, Paroxysmal nocturnal hemoglobinuria (PNH), Pernicious anemia (B12 deficiency), Polycythemia vera, Porphyria, Post-transplant lymphoproliferative disorder (PTLD), Pulmonary embolism (PE), Shwachman-Diamond syndrome (SDS), Sickle-cell disease (SCD), Thalassemia, Thrombocytopenia, Thrombotic thrombocytopenic purpura (TTP), Venous thromboembolism, Von Willebrand disease, or Waldenstrom's macroglobulinemia (lymphoplasmacytic lymphoma). In some embodiments, the blood disorder is sickle-cell disease.

In certain embodiments, the EHMT2 inhibitor is a compound of any one of Formulae (I), (I′), (I″), (II″), (III″), (I′″), (II′″), and (III′″):

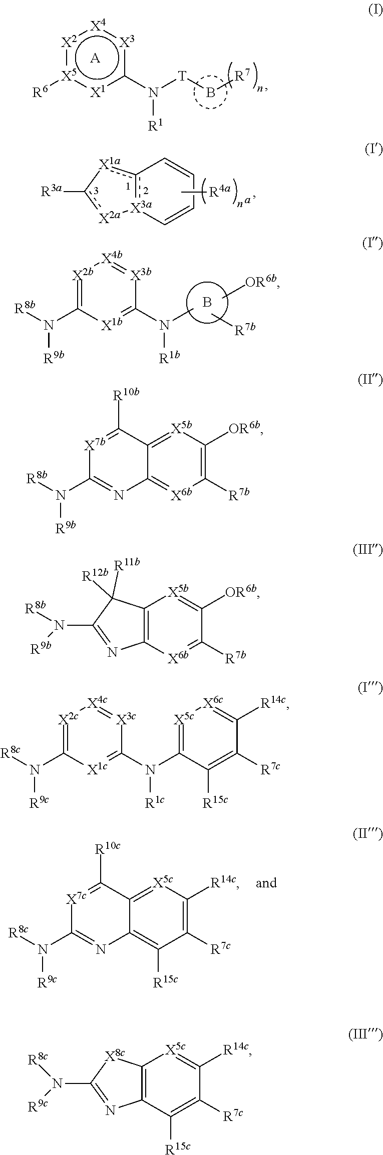

a tautomer thereof, a pharmaceutically acceptable salt of the compound, or a pharmaceutically acceptable salt of the tautomer, wherein the variables are as defined herein.

In some aspects, the present disclosure provides an EHMT2 inhibitor disclosed herein for preventing or treating a blood disorder.

In some aspects, the present disclosure provides an EHMT2 inhibitor disclosed herein for preventing or treating a blood disorder, wherein the blood disorder is Acute lymphoblastic leukemia (ALL), Acute myeloid leukemia (AML) (e.g., acute promyelocytic leukemia, APL), Amyloidosis, Anemia, Aplastic anemia, Bone marrow failure syndromes, Chronic lymphocytic leukemia (CLL), Chronic myeloid leukemia (CML), Deep vein thrombosis (DVT), Diamond-Blackfan anemia, Dyskeratosis congenita (DKC), Eosinophilic disorder, Essential thrombocythemia, Fanconi anemia, Gaucher disease, Hemochromatosis, Hemolytic anemia, Hemophilia, Hereditary spherocytosis, Hodgkin's lymphoma, Idiopathic thrombocytopenic purpura (ITP), Inherited bone marrow failure syndromes, Iron-deficiency anemia, Langerhans cell histiocytosis, Large granular lymphocytic (LGL) leukemia, Leukemia, Leukopenia, Mastocytosis, Monoclonal gammopathy, Multiple myeloma, Myelodysplastic syndromes (MDS), Myelofibrosis, Myeloproliferative neoplasms (MPN), Non-Hodgkin's lymphoma, Paroxysmal nocturnal hemoglobinuria (PNH), Pernicious anemia (B12 deficiency), Polycythemia vera, Porphyria, Post-transplant lymphoproliferative disorder (PTLD), Pulmonary embolism (PE), Shwachman-Diamond syndrome (SDS), Sickle-cell disease (SCD), Thalassemia, Thrombocytopenia, Thrombotic thrombocytopenic purpura (TTP), Venous thromboembolism, Von Willebrand disease, or Waldenstrom's macroglobulinemia (lymphoplasmacytic lymphoma).

In some aspects, the present disclosure provides an EHMT2 inhibitor disclosed herein for use in combination with one or more additional therapeutic agent for preventing or treating a blood disorder.

In some aspects, the present disclosure provides an EHMT2 inhibitor disclosed herein for use in combination with one or more additional therapeutic agent for preventing or treating a blood disorder, wherein the blood disorder is Acute lymphoblastic leukemia (ALL), Acute myeloid leukemia (AML) (e.g., acute promyelocytic leukemia, APL), Amyloidosis, Anemia, Aplastic anemia, Bone marrow failure syndromes, Chronic lymphocytic leukemia (CLL), Chronic myeloid leukemia (CML), Deep vein thrombosis (DVT), Diamond-Blackfan anemia, Dyskeratosis congenita (DKC), Eosinophilic disorder, Essential thrombocythemia, Fanconi anemia, Gaucher disease, Hemochromatosis, Hemolytic anemia, Hemophilia, Hereditary spherocytosis, Hodgkin's lymphoma, Idiopathic thrombocytopenic purpura (ITP), Inherited bone marrow failure syndromes, Iron-deficiency anemia, Langerhans cell histiocytosis, Large granular lymphocytic (LGL) leukemia, Leukemia, Leukopenia, Mastocytosis, Monoclonal gammopathy, Multiple myeloma, Myelodysplastic syndromes (MDS), Myelofibrosis, Myeloproliferative neoplasms (MPN), Non-Hodgkin's lymphoma, Paroxysmal nocturnal hemoglobinuria (PNH), Pernicious anemia (B12 deficiency), Polycythemia vera, Porphyria, Post-transplant lymphoproliferative disorder (PTLD), Pulmonary embolism (PE), Shwachman-Diamond syndrome (SDS), Sickle-cell disease (SCD), Thalassemia, Thrombocytopenia, Thrombotic thrombocytopenic purpura (TTP), Venous thromboembolism, Von Willebrand disease, or Waldenstrom's macroglobulinemia (lymphoplasmacytic lymphoma).

In some aspects, the present disclosure provides use of an EHMT2 inhibitor disclosed herein in the manufacture of a medicament for preventing or treating a blood disorder.

In some aspects, the present disclosure provides use of an EHMT2 inhibitor disclosed herein in the manufacture of a medicament for preventing or treating a blood disorder, wherein the blood disorder is Acute lymphoblastic leukemia (ALL), Acute myeloid leukemia (AML) (e.g., acute promyelocytic leukemia, APL), Amyloidosis, Anemia, Aplastic anemia, Bone marrow failure syndromes, Chronic lymphocytic leukemia (CLL), Chronic myeloid leukemia (CML), Deep vein thrombosis (DVT), Diamond-Blackfan anemia, Dyskeratosis congenita (DKC), Eosinophilic disorder, Essential thrombocythemia, Fanconi anemia, Gaucher disease, Hemochromatosis, Hemolytic anemia, Hemophilia, Hereditary spherocytosis, Hodgkin's lymphoma, Idiopathic thrombocytopenic purpura (ITP), Inherited bone marrow failure syndromes, Iron-deficiency anemia, Langerhans cell histiocytosis, Large granular lymphocytic (LGL) leukemia, Leukemia, Leukopenia, Mastocytosis, Monoclonal gammopathy, Multiple myeloma, Myelodysplastic syndromes (MDS), Myelofibrosis, Myeloproliferative neoplasms (MPN), Non-Hodgkin's lymphoma, Paroxysmal nocturnal hemoglobinuria (PNH), Pernicious anemia (B12 deficiency), Polycythemia vera, Porphyria, Post-transplant lymphoproliferative disorder (PTLD), Pulmonary embolism (PE), Shwachman-Diamond syndrome (SDS), Sickle-cell disease (SCD), Thalassemia, Thrombocytopenia, Thrombotic thrombocytopenic purpura (TTP), Venous thromboembolism, Von Willebrand disease, or Waldenstrom's macroglobulinemia (lymphoplasmacytic lymphoma).

In some aspects, the present disclosure provides use of an EHMT2 inhibitor disclosed herein in the manufacture of a medicament for use in combination with one or more additional therapeutic agent for preventing or treating a blood disorder.

In some aspects, the present disclosure provides use of an EHMT2 inhibitor disclosed herein in the manufacture of a medicament for use in combination with one or more additional therapeutic agent for preventing or treating a blood disorder, wherein the blood disorder is Acute lymphoblastic leukemia (ALL), Acute myeloid leukemia (AML) (e.g., acute promyelocytic leukemia, APL), Amyloidosis, Anemia, Aplastic anemia, Bone marrow failure syndromes, Chronic lymphocytic leukemia (CLL), Chronic myeloid leukemia (CML), Deep vein thrombosis (DVT), Diamond-Blackfan anemia, Dyskeratosis congenita (DKC), Eosinophilic disorder, Essential thrombocythemia, Fanconi anemia, Gaucher disease, Hemochromatosis, Hemolytic anemia, Hemophilia, Hereditary spherocytosis, Hodgkin's lymphoma, Idiopathic thrombocytopenic purpura (ITP), Inherited bone marrow failure syndromes, Iron-deficiency anemia, Langerhans cell histiocytosis, Large granular lymphocytic (LGL) leukemia, Leukemia, Leukopenia, Mastocytosis, Monoclonal gammopathy, Multiple myeloma, Myelodysplastic syndromes (MDS), Myelofibrosis, Myeloproliferative neoplasms (MPN), Non-Hodgkin's lymphoma, Paroxysmal nocturnal hemoglobinuria (PNH), Pernicious anemia (B12 deficiency), Polycythemia vera, Porphyria, Post-transplant lymphoproliferative disorder (PTLD), Pulmonary embolism (PE), Shwachman-Diamond syndrome (SDS), Sickle-cell disease (SCD), Thalassemia, Thrombocytopenia, Thrombotic thrombocytopenic purpura (TTP), Venous thromboembolism, Von Willebrand disease, or Waldenstrom's macroglobulinemia (lymphoplasmacytic lymphoma).

Compounds that are suitable for the methods of the disclosure include subsets of the compounds of Formulae (I), (I′), (I″), (II″), (III″), (I′″), (II′″) and specific examples that are described in U.S. Application Nos. 62/323,602, 62/348,837, 62/402,997, 62/402,863, 62/509,620, 62/436,139, 62/517,840, 62/573,442, 62/681,804, 62/746,252, and 62/746,495, and Ser. No. 15/601,888, and PCT Application Nos. PCT/US2017/027918, PCT/US2017/054468, PCT/US2017/067192, PCT/US2018/056333, and PCT/US2018/056428, the contents of each of which are incorporated herein by reference in their entireties.

In some embodiments, the method of preventing or treating a blood disorder (e.g., sickle-cell disease) comprises administering to a subject in need thereof a therapeutically effective amount of an EHMT2 inhibitor and a therapeutically effective amount of one or more additional therapeutic agent. In some embodiments, the one or more additional therapeutic agent consists of a single additional therapeutic agent. In some embodiments, the one or more additional therapeutic agent comprises a therapeutic agent provided herein. In some embodiments, the one or more additional therapeutic agent comprises a plurality of therapeutic agents, e.g., 2, 3, 4, 5, 6, 7, 8, 9, or 10 additional therapeutic agents. In some embodiments, the one or more additional therapeutic agent comprises more than 10 additional therapeutic agents.

Unless otherwise stated, any description of a method of preventing or treating embraces use of a compound, e.g., an EHMT2 inhibitor, provided herein to effect such prevention or treatment, as well as use of such compound to prepare a medicament for treating or preventing such condition. In some embodiments, the subject being treated is a human subject. In some embodiments, the subject being treated is a non-human primate. In some embodiments, the subject is a mammal, for example, a rodent. In some embodiments, the subject being treated is an animal, e.g., an animal that serves as a disease model. Methods described herein may be used to determine the efficiency of an EHMT2 inhibitor, also referred to as a candidate, in treating or preventing blood disorders. In some embodiments, the disclosure also provides methods of identifying an inhibitor of EHMT1, of EHMT2, or of both EHMT1 and EHMT2.

In some embodiments, the method further comprises the steps of performing an assay to detect a degree of protein methylation, e.g., of histone methylation, by EHMT1 and/or EHMT2 in a sample comprising blood cells from a subject in need thereof, e.g., a subject being subjected to a method provided herein, or being treated with an EHMT2 inhibitor provided herein.

In some embodiments, performing the assay to detect methylation of lysine 9 of histone 3 (H3-K9) in the histone substrate comprises measuring incorporation of labeled methyl groups.

In some embodiments, the labeled methyl groups are isotopically labeled methyl groups.

In some embodiments, performing the assay to detect methylation of H3-K9 in the histone substrate comprises contacting the histone substrate with an antibody that binds specifically to dimethylated H3-K9.

Some aspects of the disclosure provide a method of inhibiting conversion of H3-K9 to dimethylated H3-K9. In some embodiments, the method comprises contacting a mutant EHMT, a wild-type EHMT, or both, with a histone substrate comprising H3-K9 and an effective amount of a compound of the present disclosure, wherein the compound inhibits histone methyltransferase activity of EHMT, thereby inhibiting conversion of H3-K9 to dimethylated H3-K9.

Further, the compounds or methods described herein can be used for research (e.g., studying epigenetic enzymes) and other non-therapeutic purposes.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the present disclosure. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### BRIEF DESCRIPTION OF THE FIGURES

FIG. 1A-ID are a series of graphs illustrating the in vitro and in vivo studies of combining Compound 205 (an EHMT2 or G9a inhibitor) with various second agents as described in Example 3 including an exemplary dose matrix, Loewe excess model and synergy quantification by V Loewe and iobologram as well as dose response curves of Fa (fraction affected) vs log concentration of compound in the presence or absence of a combination partner and IC₅₀ of one compound vs concentration of combination partner plots (FIG. 1A), exemplary studies of synergy observed in several cell lines cotreated with Compound 205 and ATRA (FIG. 1B), exemplary studies of synergy observed in several cell lines cotreated with Compound 205 and Venetoclax (FIG. 1C), and exemplary studies of synergy observed in several cell lines cotreated with Compound 205 and DNA hypomethylating agents in 7-day cotreatment models (FIG. 1D).

FIG. 2A is a plot of cell count IC₅₀ in micromolar (μM) concentration values for all cell lines compared to type of cancer with cell lines having a cell count IC₅₀ less than 1 μM labeled demonstrating that multiple indications are sensitive to inhibition by Compound 205 in a 10-day proliferation assay and thus suitable for treatment via EHMT2 inhibition via a single agent (e.g. an EHMT2 inhibitor) as described in Example 4.

FIG. 2B is a bar graph of the number of cell lines within each type of cancer that were investigated as suitable for treatment via EHMT2 inhibition via a single agent (e.g. an EHMT2 inhibitor) as described in Example 4.

FIGS. 3A and 3B are bar graphs demonstrating the positive combinatorial effect observed for Compound 205 combined with 10 μM hydroxyurea (FIG. 3A) and observed for Compound 205 combined with 0.1 μM pomalidomide.

FIG. 4 is a series of graphs demonstrating the synergistic increase in % HbF+CD34+ cells observed by treatment with combinations of Compound 205 and hydroxyurea by FACS analysis.

FIG. 5 is a series of graphs demonstrating the synergistic increase in protein expression of Hbγ in CD34+ cells by treatment with combinations of Compound 205 and hydroxyurea by mass spectrometry analysis.

FIG. 6 is a series of graphs demonstrating the pan cellular effect Compound D5R has on human CD34+ progenitor cells isolated from SCD donors.

FIG. 7 is a series of graphs demonstrating the pan cellular combinatorial effect observed between hydroxyurea and a low dose of compound D5R.

### DETAILED DESCRIPTION

Some aspects of the present disclosure provide a method of preventing or treating a blood disorder (e.g., sickle-cell disease), the method comprising administering to a subject in need thereof a therapeutically effective amount of an EHMT2 inhibitor. In some embodiments, the EHMT2 inhibitor is a compound disclosed herein.

In certain embodiments, the blood disorder is Acute lymphoblastic leukemia (ALL), Acute myeloid leukemia (AML) (e.g., acute promyelocytic leukemia, APL), Amyloidosis, Anemia, Aplastic anemia, Bone marrow failure syndromes, Chronic lymphocytic leukemia (CLL), Chronic myeloid leukemia (CML), Deep vein thrombosis (DVT), Diamond-Blackfan anemia, Dyskeratosis congenita (DKC), Eosinophilic disorder, Essential thrombocythemia, Fanconi anemia, Gaucher disease, Hemochromatosis, Hemolytic anemia, Hemophilia, Hereditary spherocytosis, Hodgkin's lymphoma, Idiopathic thrombocytopenic purpura (ITP), Inherited bone marrow failure syndromes, Iron-deficiency anemia, Langerhans cell histiocytosis, Large granular lymphocytic (LGL) leukemia, Leukemia, Leukopenia, Mastocytosis, Monoclonal gammopathy, Multiple myeloma, Myelodysplastic syndromes (MDS), Myelofibrosis, Myeloproliferative neoplasms (MPN), Non-Hodgkin's lymphoma, Paroxysmal nocturnal hemoglobinuria (PNH), Pernicious anemia (B12 deficiency), Polycythemia vera, Porphyria, Post-transplant lymphoproliferative disorder (PTLD), Pulmonary embolism (PE), Shwachman-Diamond syndrome (SDS), Sickle-cell disease (SCD), Thalassemia, Thrombocytopenia, Thrombotic thrombocytopenic purpura (TTP), Venous thromboembolism, Von Willebrand disease, or Waldenstrom's macroglobulinemia (lymphoplasmacytic lymphoma).

In some embodiments, the blood disorder is sickle-cell anemia or beta-thalassemia. In some embodiments, the blood disease or disorder is a hematological cancer. In some embodiments, the hematological cancer is acute myeloid leukemia (AML) or chronic lymphocytic leukemia (CLL).

In some embodiments the blood disorder is sickle-cell disease (SCD).

In some embodiments, the sickle-cell disease is hemoglobin SS disease, hemoglobin SC disease, hemoglobin Sβ⁰ thalassemia disease, hemoglobin Sβ⁺ thalassemia disease, hemoglobin SD disease, or hemoglobin SE disease.

Without wishing to be bound by any theory, it is believed that sickle-cell disease describes a group of inherited red blood cell disorders in which at least some of the red blood cells of a subject having sickle-cell disease contain hemoglobin S (“HbS”). Hemoglobin S is a mutated, abnormal form of adult hemoglobin. Without wishing to be bound by any theory, it is believed that, in some embodiments, the contemplated compounds may treat sickle-cell disease by inducing fetal hemoglobin (“HbF”) expression. See, e.g., Renneville et al., Blood 126(16): 1930-1939, 2015, the content of which is incorporated herein by reference in its entirety.

In some embodiments, one or more complications of sickle-cell disease may be treated or prevented using a compound and/or a method disclosed herein. Non-limiting examples of complications that may be treated or prevented using such compounds and/or methods include anemia (e.g., severe anemia), hand-foot syndrome, splenic sequestration, delayed developmental growth, eye disorders (e.g., vision loss caused by, e.g., blockages in blood vessels supplying the eyes), skin ulcers (e.g., leg ulcers), heart disease, chest syndrome (e.g., acute chest syndrome), priapism, and pain.

Some aspects of the present disclosure provide a method of preventing or treating a blood disorder (e.g., sickle-cell disease) by administering to a subject in need thereof an effective amount of a compound of Formula (I) below:

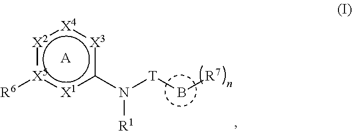

or a tautomer thereof, or a pharmaceutically acceptable salt of the compound or the tautomer, wherein
ring A is phenyl or a 5- or 6-membered heteroaryl;
X¹ is N, CR², or NR²′ as valency permits;
X² is N, CR³, or NR³′ as valency permits;
X³ is N, CR⁴, or NR⁴′ as valency permits;
X⁴ is N or CR⁵, or X⁴ is absent such that ring A is a 5-membered heteroaryl containing at least one N atom;
X⁵ is C or N as valency permits;
B is absent or a ring structure selected from the group consisting of C₆-C₁₀ aryl, C₃-C₁₀ cycloalkyl, 5- to 10-membered heteroaryl, and 4- to 12-membered heterocycloalkyl containing 1-4 heteroatoms selected from N, O, and S:
T is a bond or C₁-C₆ alkylene, C₂-C₆ alkenylene, or C₂-C₆ alkynylene linker optionally substituted with one or more of halo, cyano, hydroxyl, oxo; or C₁-C₆ alkoxy when B is present; or T is H and n is 0 when B is absent; or T is C₁-C₆ alkyl optionally substituted with (R⁷)ₙ when B is absent; or when B is absent, T and R¹ together with the atoms to which they are attached optionally form a 4-7 membered heterocycloalkyl or 5-6 membered heteroaryl, each of which is optionally substituted with (R⁷)ₙ;
R¹ is H or C₁-C₄ alkyl;
each of R², R³, and R⁴, independently is selected from the group consisting of H, halo, cyano, C₁-C₆ alkoxyl, C₆-C₁₀ aryl, NR^{a}R^{b}, C(O)NR^{a}R^{b}, NR^{a}C(O)R^{b}, C₃-C₈ cycloalkyl, 4- to 7-membered heterocycloalkyl, 5- to 6-membered heteroaryl, and C₁-C₆ alkyl, wherein C₁-C₆ alkoxyl and C₁-C₆ alkyl are optionally substituted with one or more of halo, OR, or NR^{a}R^{b}, in which each of R^{a} and R^{b} independently is H or C₁-C₆ alkyl, or R³ is -Q¹-T¹, in which Q¹ is a bond or C₁-C₆ alkylene, C₂-C₆ alkenylene, or C₂-C₆ alkynylene linker optionally substituted with one or more of halo, cyano, hydroxyl, oxo, or C₁-C₆ alkoxyl, and T¹ is H, halo, cyano, NR⁸R⁹, C(O)NR⁸R⁹, OR⁸, OR⁹, or R^{S1}, in which R^{S1} is C₃-C₈ cycloalkyl, phenyl, 4- to 12-membered heterocycloalkyl containing 1-4 heteroatoms selected from N, O, and S, or a 5- or 6-membered heteroaryl and R^{S1} is optionally substituted with one or more of halo, C₁-C₆ alkyl, hydroxyl, oxo, —C(O)R⁹, —SO₂R⁸, —SO₂N(R⁸)₂, —NR⁸C(O)R⁹, amino, mono- or di-alkylamino, or C₁-C₆ alkoxyl; or when ring A is a 5-membered heteroaryl containing at least one N atom, R⁴ is a spiro-fused 4- to 12-membered heterocycloalkyl containing 1-4 heteroatoms selected from N, O, and S;
each of R²′, R³′ and R⁴′ independently is H or C₁-C₃ alkyl;
R⁵ is selected from the group consisting of H, F, Br, cyano, C₁-C₆ alkoxyl, C₆-C₁₀ aryl, NR^{a}R^{b}, C(O)NR^{a}R^{b}, NR^{a}C(O)R^{b}, C₃-C₈ cycloalkyl, 4- to 12-membered heterocycloalkyl containing 1-4 heteroatoms selected from N, O, and S, C₁-C₆ alkyl optionally substituted with one or more of halo, OR³ or NR^{a}R^{b}, and C₂-C₆ alkynyl optionally substituted with 4- to 12-membered heterocycloalkyl; wherein said C₃-C₈ cycloalkyl or 4- to 12-membered heterocycloalkyl are optionally substituted with one or more of halo, C(O)R^{a}, OR^{a}, NR^{a}R^{b}, 4- to 7-membered heterocycloalkyl, —C₁-C₆ alkylene-4- to 7-membered heterocycloalkyl, or C₁-C₄ alkyl optionally substituted with one or more of halo, OR^{a} or NR^{a}R^{b}, in which each of R^{a} and R^{b} independently is H or C₁-C₆ alkyl; or
R⁵ and one of R³ or R⁴ together with the atoms to which they are attached form phenyl or a 5- or 6-membered heteroaryl; or R⁵ and one of R³′ or R⁴′ together with the atoms to which they are attached form a 5- or 6-membered heteroaryl, in which the phenyl or 5- or 6-membered heteroaryl as formed is optionally substituted with one or more of halo, C₁-C₃ alkyl, hydroxyl or C₁-C₃ alkoxyl;
R⁶ is absent when X⁵ is N and ring A is a 6-membered heteroaryl; or R⁶ is -Q¹-T¹, in which Q¹ is a bond or C₁-C₆ alkylene, C₂-C₆ alkenylene, or C₂-C₆ alkynylene linker optionally substituted with one or more of halo, cyano, hydroxyl, oxo, or C₁-C₆ alkoxyl, and T¹ is H, halo, cyano, NR⁸R⁹, C(O)NR⁸R⁹, C(O)R⁹, OR⁸, OR⁹, or R^{S1}, in which R^{S1} is C₃-C₈ cycloalkyl, phenyl, 4- to 12-membered heterocycloalkyl containing 1-4 heteroatoms selected from N, O, and S, or a 5- or 6-membered heteroaryl and R^{S1} is optionally substituted with one or more of halo, C₁-C₆ alkyl, hydroxyl, oxo, —C(O)R⁹, —SO₂R⁸, —SO₂N(R⁸)₂, —NR⁸C(O)R⁹, NR⁸R⁹, or C₁-C₆ alkoxyl; and R⁶ is not NR⁸C(O)NR¹²R¹³; or
R⁶ and one of R² or R³ together with the atoms to which they are attached form phenyl or a 5- or 6-membered heteroaryl; or R⁶ and one of R²′ or R³′ together with the atoms to which they are attached form a 5- or 6-membered heteroaryl, in which the phenyl or 5- or 6-membered heteroaryl as formed is optionally substituted with one or more of halo, C₁-C₃ alkyl, hydroxyl, oxo (═O), C₁-C₃ alkoxyl, or -Q¹-T¹;
each R⁷ is independently oxo (═O) or -Q²-T², in which each Q² independently is a bond or C₁-C₆ alkylene, C₂-C₆ alkenylene, or C₂-C₆ alkynylene linker optionally substituted with one or more of halo, cyano, hydroxyl, amino, mono- or di-alkylamino, or C₁-C₆ alkoxyl, and each T² independently is H, halo, cyano, OR¹⁰, OR¹¹, C(O)R¹¹, NR¹⁰R¹¹, C(O)NR¹⁰R¹¹, NR¹⁰C(O)R¹¹, 5- to 10-membered heteroaryl, C₃-C₈ cycloalkyl, or 4- to 12-membered heterocycloalkyl containing 1-4 heteroatoms selected from N, O, and S, and wherein the 5- to 10-membered heteroaryl, C₃-C₈ cycloalkyl or 4- to 12-membered heterocycloalkyl is optionally substituted with one or more of halo, C₁-C₆ alkyl optionally substituted with NR^{x}R^{y}, hydroxyl, oxo, N(R⁸)₂, cyano, C₁-C₆ haloalkyl, —SO₂R⁸, or C₁-C₆ alkoxyl, each of R^{x} and R^{y} independently being H or C₁-C₆ alkyl; and R₇ is not H or C(O)OR^{g};
each R⁸ independently is H or C₁-C₆ alkyl;
each R⁹ is independently -Q³-T³, in which Q³ is a bond or C₁-C₆ alkylene, C₂-C₆ alkenylene, or C₂-C₆ alkynylene linker optionally substituted with one or more of halo, cyano, hydroxyl, or C₁-C₆ alkoxyl, and T³ is H, halo, OR¹², OR¹³, NR¹²R¹³, NR¹²C(O)R¹³, C(O)NR¹²R¹³, C(O)R¹³, S(O)₂R¹³, S(O)₂NR¹²R¹³, or R^{S2}, in which R^{S2} is C₃-C₈ cycloalkyl, C₆-C₁₀ aryl, 4- to 12-membered heterocycloalkyl containing 1-4 heteroatoms selected from N, O, and S, or a 5- to 10-membered heteroaryl, and R^{S2} is optionally substituted with one or more -Q⁴-T⁴, wherein each Q⁴ independently is a bond or C₁-C₃ alkylene, C₂-C₃ alkenylene, or C₂-C₃ alkynylene linker each optionally substituted with one or more of halo, cyano, hydroxyl, or C₁-C₆ alkoxy, and each T⁴ independently is selected from the group consisting of H, halo, cyano, C₁-C₆ alkyl, C₃-C₈ cycloalkyl, C₆-C₁₀ aryl, 4- to 7-membered heterocycloalkyl containing 1-4 heteroatoms selected from N, O, and S, 5- to 6-membered heteroaryl, OR^{c}, C(O)R^{c}, S(O)₂R^{c}, NR^{c}R^{d}, C(O)NR^{c}R^{d}, and NR^{c}C(O)R^{d}, each of R^{c} and R^{d} independently being H or C₁-C₆ alkyl; or -Q⁴-T⁴ is oxo; or
R⁸ and R⁹ taken together with the nitrogen atom to which they are attached form a 4- to 12-membered heterocycloalkyl containing 1-4 heteroatoms selected from N, O and S, which is optionally substituted with one or more of -Q⁵-T⁵, wherein each Q⁵ independently is a bond or C₁-C₃ alkylene, C₂-C₃ alkenylene, or C₂-C₃ alkynylene linker each optionally substituted with one or more of halo, cyano, hydroxyl, or C₁-C₆ alkoxy, and each T independently is selected from the group consisting of H, halo, cyano, C₁-C₆ alkyl, C₃-C₈ cycloalkyl, C₆-C₁₀ aryl, 4- to 7-membered heterocycloalkyl containing 1-4 heteroatoms selected from N, O, and S, 5- to 6-membered heteroaryl, OR^{e}, C(O)R^{e}, S(O)₂R^{e}, S(O)₂NR^{e}R^{f}, NR^{e}R^{f}, C(O)NR^{e}R^{f}, and NR^{e}C(O)R^{f}, each of R^{e} and R^{f} independently being H or C₁-C₆ alkyl; or -Q⁵-T⁵ is oxo;
R¹⁰ is selected from the group consisting of H and C₁-C₆ alkyl;
R¹¹ is -Q⁶-T⁶, in which Q⁶ is a bond or C₁-C₆ alkylene, C₂-C₆ alkenylene, or C₂-C₆ alkynylene linker optionally substituted with one or more of halo, cyano, hydroxyl, oxo, or C₁-C₆ alkoxyl, and T⁶ is H, halo, OR^{g}, NR^{g}R^{h}, NR^{g}C(O)R^{h}, C(O)NR^{g}R^{h}, C(O)R^{g}, S(O)₂R^{g}, or R^{S3}, in which each of R^{g} and R^{h} independently is H, phenyl, C₃-C₈ cycloalkyl, or C₁-C₆ alkyl optionally substituted with C₃-C₈ cycloalkyl, or R^{g} and R^{h} together with the nitrogen atom to which they are attached form a 4- to 12-membered heterocycloalkyl containing 1-4 heteroatoms selected from N, O, and S, and R^{S3} is C₃-C₈ cycloalkyl, C₆-C₁₀ aryl, 4- to 12-membered heterocycloalkyl containing 1-4 heteroatoms selected from N, O and S, or a 5- to 10-membered heteroaryl, and R^{S3} is optionally substituted with one or more -Q⁷-T⁷, wherein each Q⁷ independently is a bond or C₁-C₃ alkylene, C₂-C₃ alkenylene, or C₂-C₃ alkynylene linker each optionally substituted with one or more of halo, cyano, hydroxyl, or C₁-C₆ alkoxy, and each T⁷ independently is selected from the group consisting of H, halo, cyano, C₁-C₆ alkyl, C₃-C₈ cycloalkyl, C₆-C₁₀ aryl, 4- to 7-membered heterocycloalkyl containing 1-4 heteroatoms selected from N, O, and S, 5- to 6-membered heteroaryl, OR^{j}, C(O)R^{j}, NR^{j}R^{k}, C(O)NR^{j}R^{k}, S(O)₂R^{j}, and NR^{j}C(O)R^{k}, each of R^{j} and R^{k} independently being H or C₁-C₆ alkyl optionally substituted with one or more halo; or -Q⁷-T⁷ is oxo; or
R¹⁰ and R¹¹ taken together with the nitrogen atom to which they are attached form a 4- to 12-membered heterocycloalkyl containing 1-4 heteroatoms selected from N, O, and S, which is optionally substituted with one or more of halo, C₁-C₆ alkyl, hydroxyl, or C₁-C₆ alkoxyl;
R¹² is H or C₁-C₆ alkyl;
R¹³ is C₁-C₆ alkyl, C₃-C₈ cycloalkyl, C₆-C₁₀ aryl, 4- to 12-membered heterocycloalkyl containing 1-4 heteroatoms selected from N, O, and S, or a 5- to 10-membered heteroaryl, each of which is optionally substituted with one or more -Q⁸-T⁸, wherein each Q⁸ independently is a bond or C₁-C₃ alkylene, C₂-C₃ alkenylene, or C₂-C₃ alkynylene linker each optionally substituted with one or more of halo, cyano, hydroxyl, or C₁-C₆ alkoxy, and each T⁸ independently is selected from the group consisting of H, halo, cyano, C₁-C₆ alkyl, C₃-C₈ cycloalkyl, C₆-C₁₀ aryl, 4- to 7-membered heterocycloalkyl containing 1-4 heteroatoms selected from N, O, and S, and 5- to 6-membered heteroaryl; or -Q⁸-T⁸ is oxo; and
n is 0, 1, 2, 3, or 4, provided that
the compound of Formula (I) is not
2-cyclohexyl-6-methoxy-N-[1-(1-methylethyl)-4-piperidinyl]-7-[3-(1-pyrrolidinyl)propoxy]-4-quinazolinamine;
N-(1-isopropylpiperidin-4-yl)-6-methoxy-2-(4-methyl-1,4-diazepan-1-yl)-7-(3-(piperidin-1-yl)propoxy)quinazolin-4-amine;
2-(4,4-difluoropiperidin-1-yl)-N-(1-isopropylpiperidin-4-yl)-6-methoxy-7-(3-(pyrrolidin-1-yl)propoxy)quinazolin-4-amine; or
2-(4-isopropyl-1,4-diazepan-1-yl)-N-(1-isopropylpiperidin-4-yl)-6-methoxy-7-(3-(piperidin-1-yl)propoxy)quinazolin-4-amine.

The compounds of Formula (I) may have one or more of the following features when applicable.

In some embodiments, the EHMT2-inhibitor is not a compound selected from the group consisting of:
4-(((2-((1-acetylindolin-6-yl)amino)-6-(trifluoromethyl)pyrimidin-4-yl)amino)methyl)benzenesulfonamide;
5-bromo-N⁴-(4-fluorophenyl)-N²-(4-methoxy-3-(2-(pyrrolidin-1-yl)ethoxy)phenyl)pyrimidine-2,4-diamine;
N²-(4-methoxy-3-(2-(pyrrolidin-1-yl)ethoxy)phenyl)-N⁴-(5-(tert-pentyl)-1H-pyrazol-3-yl)pyrimidine-2,4-diamine;
4-((2,4-dichloro-5-methoxyphenyl)amino)-2-((3-(2-(pyrrolidin-1-yl)ethoxy)phenyl)amino)pyrimidine-5-carbonitrile;
N-(naphthalen-2-yl)-2-(piperidin-1-ylmethoxy)pyrimidin-4-amine;
N-(3,5-difluorobenzyl)-2-(3-(pyrrolidin-1-yl)propyl)pyrimidin-4-amine;
N-(((4-(3-(piperidin-1-yl)propyl)pyrimidin-2-yl)amino)methyl)benzamide;
N-(2-((2-(3-(dimethylamino)propyl)pyrimidin-4-yl)amino)ethyl)benzamide; and
2-(hexahydro-4-methyl-1H-1,4-diazepin-1-yl)-6,7-dimethoxy-N-[1-(phenylmethyl)-4-piperidinyl]-4-quinazolinamine;

In some embodiments, when T is a bond, B is substituted phenyl, and R⁶ is NR⁸R⁹, in which R⁹ is -Q³-R^{S2}, and R^{S2} is optionally substituted 4- to 7-membered heterocycloalkyl or a 5- to 6-membered heteroaryl, then B is substituted with at least one substituent selected from (i) -Q²-OR¹¹ in which R¹¹ is -Q⁶-R^{S3} and Q⁶ is optionally substituted C₂-C₆ alkylene, C₂-C₆ alkenylene, or C₂-C₆ alkynylene linker and (ii) -Q²-NR¹⁰R¹¹ in which R¹¹ is -Q⁶-R^{S3};

In some embodiments, when T is a bond and B is optionally substituted phenyl, then R⁶ is not OR⁹ or NR⁸R⁹ in which R⁹ is optionally substituted naphthyl;

In some embodiments, when T is a bond and B is optionally substituted phenyl, naphthyl, indanyl or 1,2,3,4-tetrahydronaphthyl, then R⁶ is not NR⁸R⁹ in which R⁹ is optionally substituted phenyl, naphthyl, indanyl or 1,2,3,4-tetrahydronaphthyl;

In some embodiments, when T is a bond and B is optionally substituted phenyl or thiazolyl, then R⁶ is not optionally substituted imidazolyl, pyrazolyl, pyridyl, pyrimidyl, or NR⁸R⁹ in which R⁹ is optionally substituted imidazolyl or 6- to 10-membered heteroaryl; or

In some embodiments, when T is a C₁-C₆ alkylene linker and B is absent or optionally substituted C₆-C₁₀ aryl or 4- to 12-membered heterocycloalkyl; or when T is a bond and B is optionally substituted C₃-C₁₀ cycloalkyl or 4- to 12-membered heterocycloalkyl, then R⁶ is not NR⁸C(O)R³;

In some embodiments, when X¹ and X³ are N, X² is CR³, X⁴ is CR⁵, X⁵ is C, R⁵ is 4- to 12-membered heterocycloalkyl substituted with one or more C₁-C₆ alkyl, and R⁶ and R³ together with the atoms to which they are attached form phenyl which is substituted with one or more of optionally substituted C₁-C₃ alkoxyl, then B is absent, C₆-C₁₀ aryl, C₃-C₁₀ cycloalkyl, or 5- to 10-membered heteroaryl, or

In some embodiments, when X² and X³ are N, X¹ is CR², X⁴ is CR⁵, X⁵ is C, R⁵ is C₃-C₈ cycloalkyl or 4- to 12-membered heterocycloalkyl, each optionally substituted with one or more C₁-C₆ alkyl, and R⁶ and R² together with the atoms to which they are attached form phenyl which is substituted with one or more of optionally substituted C₁-C₃ alkoxyl, then B is absent, C₆-C₁₀ aryl, C₃-C₁₀ cycloalkyl, or 5- to 10-membered heteroaryl.

In some embodiments, ring A is a 6-membered heteroaryl, at least one of X¹, X², X³ and X⁴ is N and X⁵ is C.

In some embodiments, ring A is a 6-membered heteroaryl, two of X¹, X², X³ and X⁴ are N and X⁵ is C.

In some embodiments, R⁶ and one of R² or R³ together with the ring A to which they are attached form a 6,5-fused bicyclic heteroaryl; or R⁶ and one of R^{2′} or R^{3′} together the ring A to which they are attached form a 6,5-fused bicyclic heteroaryl.

In some embodiments, at least one of R⁶, R², R³, and R⁴ is not H.

In some embodiments, when one or more of R²′, R³′, and R⁴′ are present, at least one of R⁶, R²′, R³′, and R⁴′ is not H.

In some embodiments, the EHMT2 inhibitor is a compound of Formula (II):

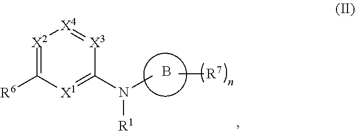

wherein
ring B is phenyl or pyridyl,
one or both of X¹ and X² are N while X³ is CR⁴ and X⁴ is CR⁵ or one or both of X¹ and X³ are N while X² is CR³ and X⁴ is CR⁵; and
n is 1, 2, or 3.

In some embodiments, the EHMT2 inhibitor is a compound of Formula (IIa1), (IIa2), (IIa3), (IIa4), or (IIa5):

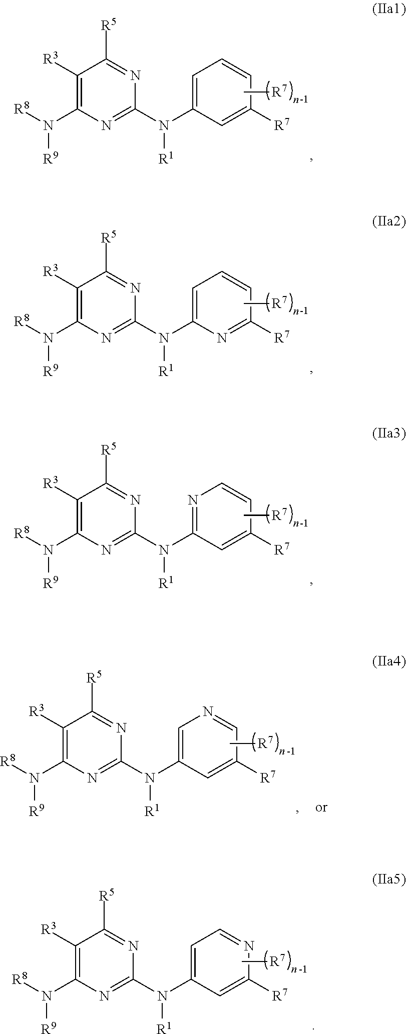

In some embodiments, at most one of R³ and R⁵ is not H.

In some embodiments, the EHMT2 inhibitor is a compound of Formula (IIb1), (IIb2), (IIb3), (IIb4), or (IIb5):

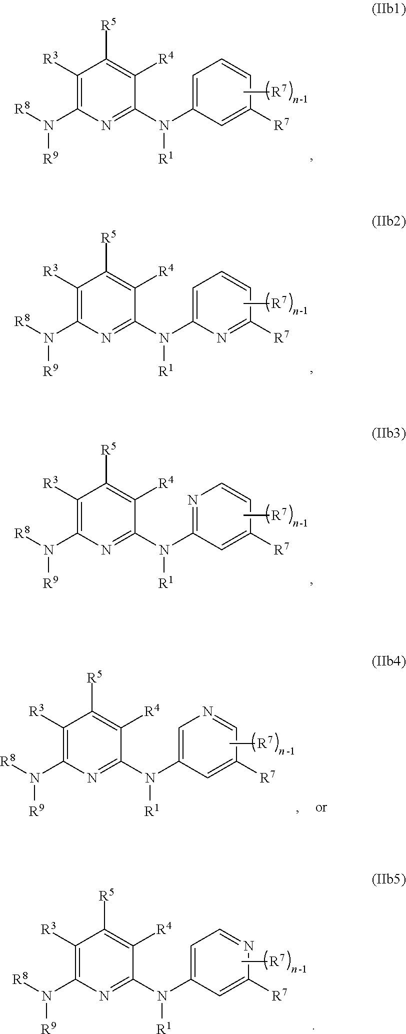

In some embodiments, at most one of R³, R⁴ and R⁵ is not H.

In some embodiments, the EHMT2 inhibitor is a compound of Formula (IIc1), (IIc2), (IIc3), (IIc4), or (IIc5):

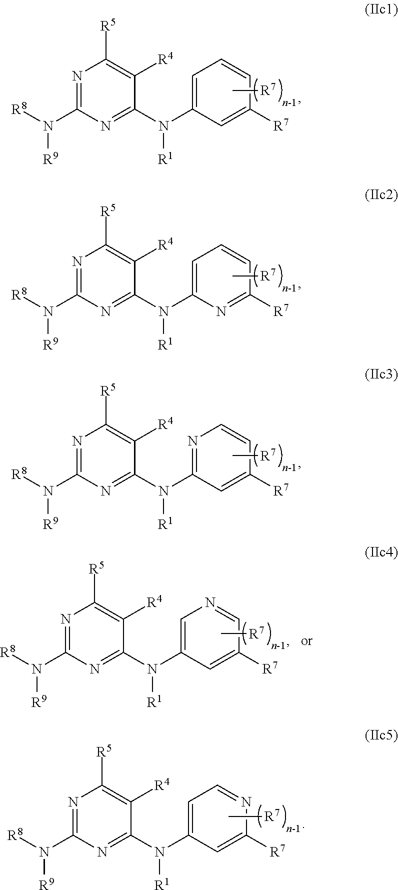

In some embodiments, at most one of R⁴ and R⁵ is not H.

In some embodiments, the EHMT2 inhibitor is a compound of Formula (IId1), (IId2), (IId3), (IId4), or (IId5):

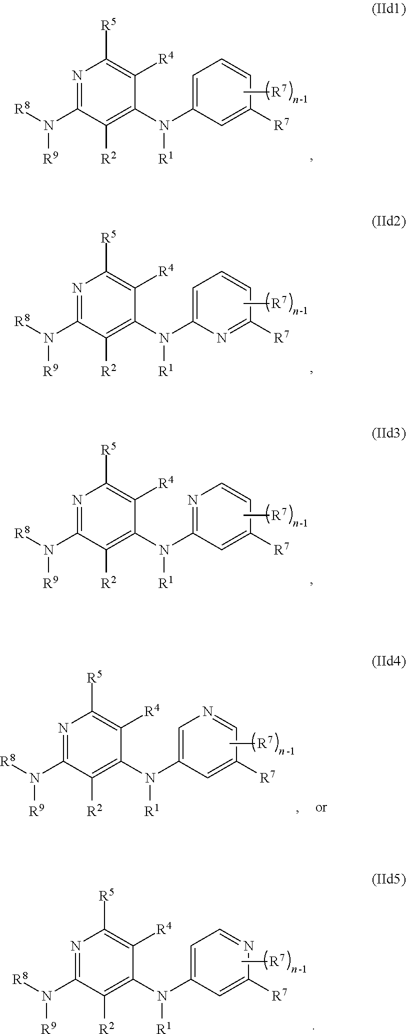

In some embodiments, at most one of R², R⁴, and R⁵ is not H.

In some embodiments, ring A is a 5-membered heteroaryl.

In some embodiments, the EHMT2 inhibitor is a compound of Formula (III):

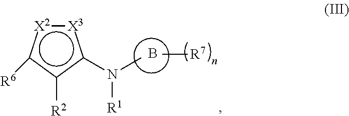

wherein
ring B is phenyl or pyridyl,
at least one of X² and X³ is N; and
n is 1 or 2.

In some embodiments, the EHMT2 inhibitor is a compound of Formula (IIIa):

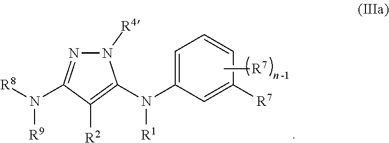

In some embodiments, at most one of R⁴′ and R² is not H.

In some embodiments, the optionally substituted 6,5-fused bicyclic heteroaryl contains 1-4 N atoms.

In some embodiments, T is a bond and ring B is phenyl or pyridyl.

In some embodiments, n is 1 or 2.

In some embodiments, the EHMT2 inhibitor is a compound of Formula (IV):

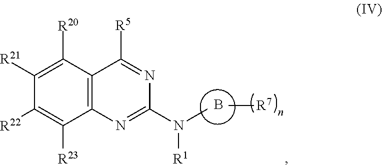

wherein
ring B is C₃-C₆ cycloalkyl;
each of R²⁰, R²¹, R²² and R²³ independently is H, halo, C₁-C₃ alkyl, hydroxyl, or C₁-C₃ alkoxyl; and
n is 1 or 2.

In some embodiments, ring B is cyclohexyl.

In some embodiments, R¹ is H or CH₃.

In some embodiments, n is 1 or 2, and at least one of R⁷ is -Q²-OR¹¹ in which R¹¹ is -Q⁶-R^{S3} and Q⁶ is optionally substituted C₂-C₆ alkylene, C₂-C₆ alkenylene, or C₂-C₆ alkynylene linker.

In some embodiments, n is 1 or 2, and at least one of R⁷ is -Q²-NR¹⁰R¹¹ in which R¹¹ is -Q⁶-R^{S3}.

In some embodiments, Q⁶ is C₂-C₆ alkylene, C₂-C₆ alkenylene, or C₂-C₆ alkynylene linker optionally substituted with a hydroxyl and R^{S3} is 4- to 7-membered heterocycloalkyl optionally substituted with one or more -Q⁷-T⁷.

In some embodiments, Q⁶ is C₁-C₆ alkylene, C₂-C₆ alkenylene, or C₂-C₆ alkynylene linker optionally substituted with a hydroxyl and R^{S3} is C₃-C₆ cycloalkyl optionally substituted with one or more -Q⁷-T⁷.

In some embodiments, each Q⁷ is independently a bond or a C₁-C₃ alkylene, C₂-C₃ alkenylene, or C₂-C₃ alkynylene linker and each T⁷ is independently H, halo, C₁-C₆ alkyl, or phenyl.

In some embodiments, Q² is a bond or a C₁-C₄ alkylene, C₂-C₄ alkenylene, or C₂-C₄ alkynylene linker.

In some embodiments, at least one of R⁷ is

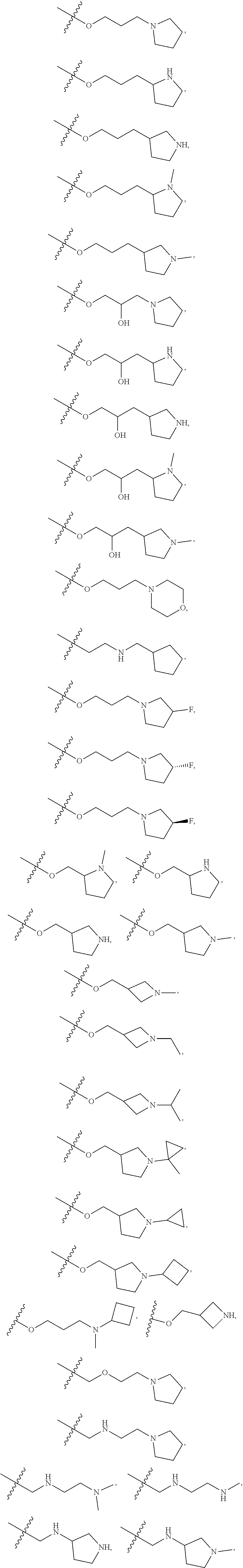

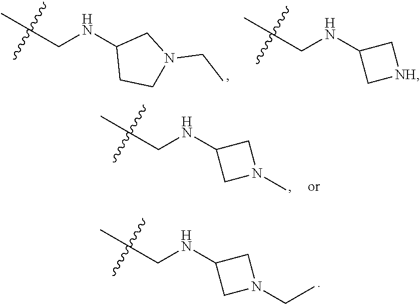

In some embodiments, n is 2 and the compound further comprises another R selected from halo and methoxy.

In some embodiments, ring B is selected from phenyl, pyridyl, and cyclohexyl, and the halo or methoxy is at the para-position to NR¹.

In some embodiments, R⁶ is NR⁸R⁹.

In some embodiments, R⁹ is -Q³-T³, in which T³ is OR¹², NR¹²C(O)R¹³, C(O)R¹³, C(O)NR¹²R¹³, S(O)₂NR¹²R¹³, or R^{S2}.

In some embodiments, Q³ is C₁-C₆ alkylene, C₂-C₆ alkenylene, or C₂-C₆ alkynylene linker optionally substituted with a hydroxyl.

In some embodiments, R^{S2} is C₃-C₆ cycloalkyl, phenyl, 4- to 12-membered heterocycloalkyl, or a 5- to 10-membered heteroaryl, and R^{S2} is optionally substituted with one or more-Q⁴-T⁴.

In some embodiments, each Q⁴ is independently a bond or C₁-C₃ alkylene, C₂-C₃ alkenylene, or C₂-C₃ alkynylene linker optionally substituted with one or more of hydroxyl and halo, and each T⁴ is independently H, halo, C₁-C₆ alkyl, or phenyl; or -Q⁴-T⁴ is oxo.

In some embodiments, R⁶ or NR⁸R⁹ is selected from the group consisting of:

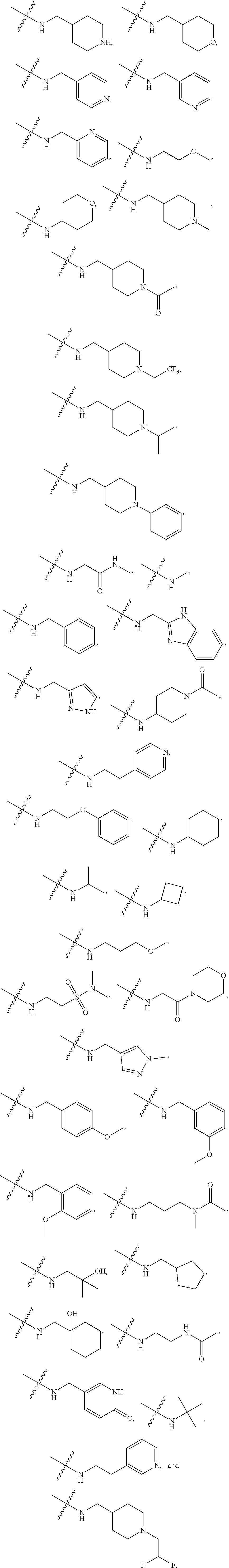

In some embodiments, B is absent and T is unsubstituted C₁-C₆ alkyl or T is C₁-C₆ alkyl substituted with at least one R⁷.

In some embodiments, B is 4- to 12-membered heterocycloalkyl and T is unsubstituted C₁-C₆ alkyl.

In some embodiments, the EHMT2 inhibitor is a compound of Formula (V):

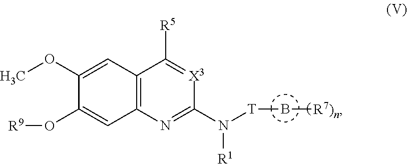

wherein
ring B is absent or C₃-C₆ cycloalkyl;
X³ is N or CR⁴ in which R⁴ is H or C₁-C₄ alkyl;
R¹ is H or C₁-C₄ alkyl;
or when B is absent, T and R¹ together with the atoms to which they are attached optionally form a 4-7 membered heterocycloalkyl or 5-6 membered heteroaryl, each of which is optionally substituted with (R⁷)ₙ; or when B is absent, T is H and n is 0;
each R⁷ is independently oxo (═O) or -Q²-T², in which each Q² independently is a bond or C₁-C₆ alkylene, C₂-C₆ alkenylene, or C₂-C₆ alkynylene linker optionally substituted with one or more of halo, cyano, hydroxyl, amino, mono- or di-alkylamino, or C₁-C₆ alkoxyl, and each T² independently is H, halo, OR¹⁰, OR¹¹, C(O)R¹¹, NR¹⁰R¹¹, C(O)NR¹⁰R¹¹, NR¹⁰C(O)R¹¹, C₃-C₈ cycloalkyl, or 4- to 12-membered heterocycloalkyl containing 1-4 heteroatoms selected from N, O, and S, and wherein the C₃-C₈ cycloalkyl or 4- to 12-membered heterocycloalkyl is optionally substituted with one or more of halo, C₁-C₆ alkyl optionally substituted with NR^{x}R^{y}, hydroxyl, oxo, N(R⁸)₂, cyano, C₁-C₆ haloalkyl, —SO₂R⁸, or C₁-C₆ alkoxyl, each of R^{x} and R^{y} independently being H or C₁-C₆ alkyl; and R⁷ is not H or C(O)OR⁹:
R⁵ is selected from the group consisting of C₁-C₆ alkyl, C₃-C₈ cycloalkyl and 4- to 12-membered heterocycloalkyl containing 1-4 heteroatoms selected from N, O and S, wherein the C₃-C₈ cycloalkyl and 4- to 12-membered heterocycloalkyl is optionally substituted with one or more of 4- to 7-membered heterocycloalkyl, —C₁-C₆ alkylene-4- to 7-membered heterocycloalkyl, —C(O)C₁-C₆ alkyl or C₁-C₆ alkyl optionally substituted with one or more of halo or OR^{a};
R⁹ is -Q³-T³, in which Q³ is a bond or C₁-C₆ alkylene, C₂-C₆ alkenylene, or C₂-C₆ alkynylene linker optionally substituted with one or more of halo, cyano, hydroxyl, or C₁-C₆ alkoxyl, and T³ is 4- to 12-membered heterocycloalkyl containing 1-4 heteroatoms selected from N, O, and S, optionally substituted with one or more -Q⁴-T⁴, wherein each Q⁴ independently is a bond or C₁-C₃ alkylene, C₂-C₃ alkenylene, or C₂-C₃ alkynylene linker each optionally substituted with one or more of halo, cyano, hydroxyl, or C₁-C₆ alkoxy, and each T⁴ independently is selected from the group consisting of H, halo, cyano, C₁-C₆ alkyl, C₃-C₈ cycloalkyl, C₆-C₁₀ aryl, 4- to 7-membered heterocycloalkyl containing 1-4 heteroatoms selected from N, O, and S, 5- to 6-membered heteroaryl, OR^{c}, C(O)R^{c}, S(O)₂R^{c}, NR^{c}R^{d}, C(O)NR^{c}R^{d}, and NR^{c}C(O)R^{d} each of R^{c} and R^{d} independently being H or C₁-C₆ alkyl; or -Q⁴-T⁴ is oxo; and
n is 0, 1 or 2.

In some embodiments, the EHMT2 inhibitor is a compound of Formula (VI):

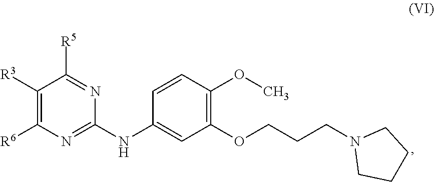

wherein
R⁵ and R⁶ are independently selected from the group consisting of C₁-C₆ alkyl and NR⁸R⁹, or R⁶ and R³ together with the atoms to which they are attached form phenyl or a 5- or 6-membered heteroaryl.

In some embodiments, R⁶ is methyl.

In some embodiments, the EHMT2 inhibitor is a compound of Formula (VII):

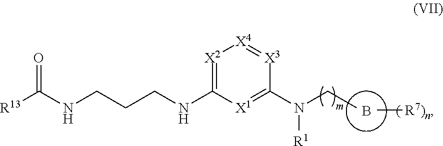

wherein m is 1 or 2 and n is 0, 1, or 2.

In some embodiments, both of X¹ and X³ are N while X² is CR³ and X⁴ is CR⁵.

In some embodiments, the EHMT2 inhibitor is a compound of Formula (VIIIa):

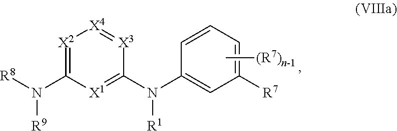

wherein
X¹ is N or CR²;
X² is N or CR³;
X³ is N or CR⁴;
X⁴ is N or CR⁵:
R² is selected from the group consisting of H, C₃-C₈ cycloalkyl, and C₁-C₆ alkyl optionally substituted with one or more of halo, OR^{a}, or N^{a}R^{b};
each of R³ and R⁴ is H; and
R⁵ are independently selected from the group consisting of H, C₃-C₈ cycloalkyl, and C₁-C₆ alkyl optionally substituted with one or more of halo or OR^{a}; or
R⁵ and one of R³ or R⁴ together with the atoms to which they are attached form phenyl or a 5- or 6-membered heteroaryl; or R⁵ and one of R³′ or R⁴′ together with the atoms to which they are attached form a 5- or 6-membered heteroaryl, in which the phenyl or 5- or 6-membered heteroaryl as formed is optionally substituted with one or more of halo, C₁-C₃ alkyl, hydroxyl or C₁-C₃ alkoxyl; and
wherein at least one of R₂ or R₅ are not H.

In some embodiments, the EHMT2 inhibitor is a compound of Formula (VIIIb):

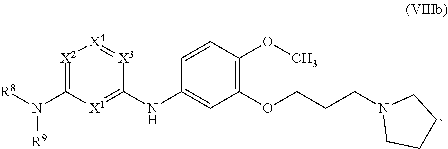

wherein
X¹ is N or CR²;
X² is N or CR³;
X³ is N or CR⁴;
X⁴ is N or CR⁵;
R² is selected from the group consisting of H, C₃-C₈ cycloalkyl, and C₁-C₆ alkyl each of R³ and R⁴ is H; and
R⁵ is selected from the group consisting of H, C₃-C₈ cycloalkyl, and C₁-C₆ alkyl; or
R⁵ and one of R³ or R⁴ together with the atoms to which they are attached form phenyl or a 5- or 6-membered heteroaryl; or R⁵ and one of R³′ or R⁴′ together with the atoms to which they are attached form a 5- or 6-membered heteroaryl, in which the phenyl or 5- or 6-membered heteroaryl as formed is optionally substituted with one or more of halo, C₁-C₃ alkyl, hydroxyl or C₁-C₃ alkoxyl; and
wherein at least one of R₂ or R₅ are not H.

In some embodiments, the EHMT2 inhibitor is a compound of Formula (VIIIc):

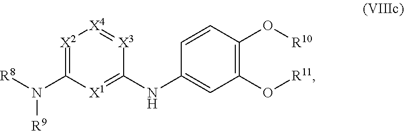

wherein
X¹ is N or CR²;
X² is N or CR³;
X³ is N or CR⁴;
X⁴ is N or CR⁵:
R² is selected from the group consisting of H, C₃-C₈ cycloalkyl, and C₁-C₆ alkyl each of R³ and R⁴ is H; and
R⁵ is selected from the group consisting of H, C₃-C₈ cycloalkyl, and C₁-C₆ alkyl; or
R⁵ and one of R³ or R⁴ together with the atoms to which they are attached form phenyl or a 5- or 6-membered heteroaryl; or R⁵ and one of R³′ or R⁴′ together with the atoms to which they are attached form a 5- or 6-membered heteroaryl, in which the phenyl or 5- or 6-membered heteroaryl as formed is optionally substituted with one or more of halo, C₁-C₃ alkyl, hydroxyl or C₁-C₃ alkoxyl; and
wherein at least one of R₂ or R₅ are not H.

In some embodiments, the EHMT2 inhibitor is a compound of (IX):

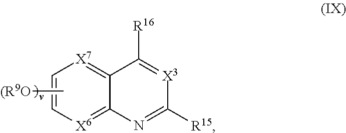

or a tautomer thereof, or a pharmaceutically acceptable salt of the compound or the tautomer, wherein
X⁶ is N or CH;
X⁷ is N or CH;
X³ is N or CR⁴;
R⁴, independently is selected from the group consisting of H, halo, cyano, C₁-C₆ alkoxyl, C₆-C₁₀ aryl, NR^{a}R^{b}, C(O)NR^{a}R, NR^{a}C(O)R^{b}, C₃-C₈ cycloalkyl, 4- to 7-membered heterocycloalkyl, 5- to 6-membered heteroaryl, and C₁-C₆ alkyl, wherein C₁-C₆ alkoxyl and C₁-C₆ alkyl are optionally substituted with one or more of halo, OR^{a}, or NR^{a}R^{b}, in which each of R^{a} and R^{b} independently is H or C₁-C₆ alkyl;
each R⁹ is independently -Q³-T³, in which Q³ is a bond or C₁-C₆ alkylene, C₂-C₆ alkenylene, or C₂-C₆ alkynylene linker optionally substituted with one or more of halo, cyano, hydroxyl, or C₁-C₆ alkoxyl, and T³ is H, halo, OR¹², OR¹³, NR¹²R¹³, NR¹²C(O)R¹³, C(O)NR¹²R¹³, C(O)R¹³, S(O)₂R¹³, S(O)₂NR¹²R¹³, or R^{S2}, in which R^{S2} is C₃-C₈ cycloalkyl, C₆-C₁₀ aryl, 4- to 12-membered heterocycloalkyl containing 1-4 heteroatoms selected from N, O, and S, or a 5- to 10-membered heteroaryl, and R^{S2} is optionally substituted with one or more -Q⁴-T⁴, wherein each Q⁴ independently is a bond or C₁-C₃ alkylene, C₂-C₃ alkenylene, or C₂-C₃ alkynylene linker each optionally substituted with one or more of halo, cyano, hydroxyl, or C₁-C₆ alkoxy, and each T⁴ independently is selected from the group consisting of H, halo, cyano, C₁-C₆ alkyl, C₃-C₈ cycloalkyl, C₆-C₁₀ aryl, 4- to 7-membered heterocycloalkyl containing 1-4 heteroatoms selected from N, O, and S, 5- to 6-membered heteroaryl, OR^{c}, C(O)R^{c}, S(O)₂R^{c}, NR^{c}R^{d}, C(O)NR^{c}R^{d}, and NR^{c}C(O)R^{d}, each of R^{c} and R^{d} independently being H or C₁-C₆ alkyl; or -Q⁴-T⁴ is oxo; or
R¹² is H or C₁-C₆ alkyl;
R¹³ is C₁-C₆ alkyl, C₃-C₈ cycloalkyl, C₆-C₁₀ aryl, 4- to 12-membered heterocycloalkyl containing 1-4 heteroatoms selected from N, O, and S, or a 5- to 10-membered heteroaryl, each of which is optionally substituted with one or more -Q⁸-T⁸, wherein each Q⁸ independently is a bond or C₁-C₃ alkylene, C₂-C₃ alkenylene, or C₂-C₃ alkynylene linker each optionally substituted with one or more of halo, cyano, hydroxyl, or C₁-C₆ alkoxy, and each T⁸ independently is selected from the group consisting of H, halo, cyano, C₁-C₆ alkyl, C₃-C₈ cycloalkyl, C₆-C₁₀ aryl, 4- to 7-membered heterocycloalkyl containing 1-4 heteroatoms selected from N, O, and S, and 5- to 6-membered heteroaryl; or -Q⁸-T⁸ is oxo;
R¹⁵ is C₁-C₆ alkyl, NHR¹⁷, C₃-C₈ cycloalkyl, C₆-C₁₀ aryl, 4- to 12-membered heterocycloalkyl containing 1-4 heteroatoms selected from N, O, and S, or 5- to 10-membered heteroaryl, wherein each of said C₁-C₆ alkyl, C₃-C₈ cycloalkyl, C₆-C₁₀ aryl, 4- to 12-membered heterocycloalkyl, and 5- to 10-membered heteroaryl is optionally substituted with one or more -Q⁹-T⁹, wherein each Q⁹ independently is a bond or C₁-C₃ alkylene, C₂-C₃ alkenylene, or C₂-C₃ alkynylene linker each optionally substituted with one or more of halo, cyano, hydroxyl, or C₁-C₆ alkoxy, and each T⁹ independently is selected from the group consisting of H, halo, cyano, C₁-C₆ alkyl, C₃-C₈ cycloalkyl, C₆-C₁₀ aryl, 4- to 7-membered heterocycloalkyl containing 1-4 heteroatoms selected from N, O, and S, and 5- to 6-membered heteroaryl; or -Q⁹-T⁹ is oxo;
R¹⁶ is C₁-C₆ alkyl, C₂-C₆ alkenyl, C₂-C₆ alkynyl, C₃-C₈ cycloalkyl, C₆-C₁₀ aryl, 4- to 12-membered heterocycloalkyl containing 1-4 heteroatoms selected from N, O, and S, or a 5- to 10-membered heteroaryl, each of which is optionally substituted with one or more -Q¹⁰-T¹⁰, wherein each Q¹⁰ independently is a bond or C₁-C₃ alkylene, C₂-C₃ alkenylene, or C₂-C₃ alkynylene linker each optionally substituted with one or more of halo, cyano, hydroxyl, or C₁-C₆ alkoxy, and each T¹⁰ independently is selected from the group consisting of H, halo, cyano, C₁-C₆ alkyl, C₃-C₈ cycloalkyl, C₆-C₁₀ aryl, 4- to 7-membered heterocycloalkyl containing 1-4 heteroatoms selected from N, O, and S, and 5- to 6-membered heteroaryl; or -Q¹⁰-T¹⁰ is oxo;
R¹⁷ is H or C₁-C₆ alkyl; and
v is 0, 1, or 2.

In some embodiments, each T³ independently is OR¹² or OR¹².

In some embodiments, each Q³ independently is a bond or C₁-C₆ alkylene, C₂-C₆ alkenylene, or C₂-C₆ alkynylene linker optionally substituted with a hydroxyl.

In some embodiments, R¹⁵ is C₁-C₆ alkyl, NHR¹⁷, or 4- to 12-membered heterocycloalkyl.

In some embodiments, R¹⁶ is C₁-C₆ alkyl or 4- to 12-membered heterocycloalkyl, each optionally substituted with one or more -Q¹⁰-T¹⁰.

In some embodiments, each T¹⁰ independently is selected from the group consisting of H, halo, cyano, C₁-C₆ alkyl, and 4- to 7-membered heterocycloalkyl.

In some embodiments, each Q¹⁰ independently is a bond or C₁-C₃ alkylene, C₂-C₃ alkenylene, or C₂-C₃ alkynylene linker optionally substituted with a hydroxyl.

In some embodiments, the EHMT2 inhibitor is a compound of Formula (X):

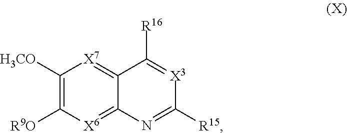

wherein X³ is N or CR⁴, wherein R⁴ is selected from the group consisting of H, halo, and cyano.

In some embodiments, the EHMT2 inhibitor is a compound of Formula (Xa), (Xb), (Xc), (Xd), (Xe), (Xf), or (Xg):

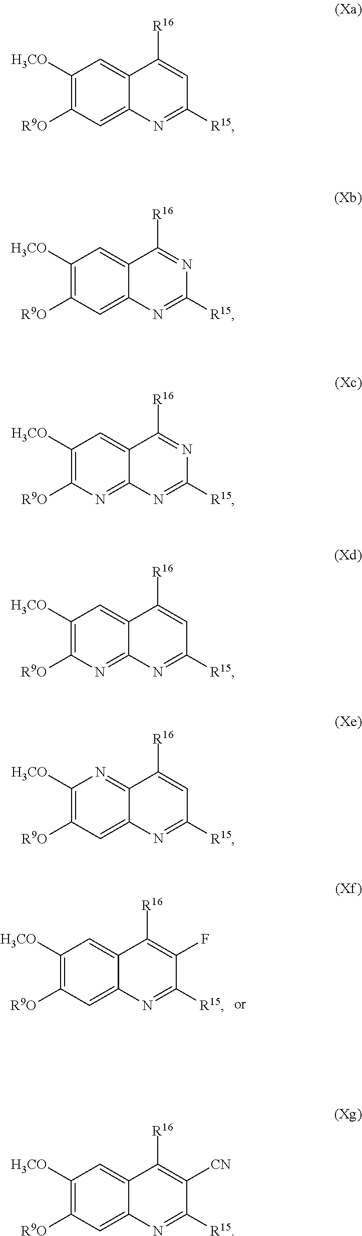

In some embodiments, at least one of X¹, X², X³ and X⁴ is N.

In some embodiments, X² and X³ is CH, and X¹ and X⁴ is N.

In some embodiments, X² and X³ is N, X¹ is CR², and X⁴ is CR⁵.

In some embodiments, R⁶ is NR⁸R⁹ and R⁵ is C₁₋₆ alkyl or R⁵ and R³ together with the atoms to which they are attached form phenyl or a 5- to 6-membered heteroaryl ring.

In another aspect, the present disclosure provides a method of preventing or treating a blood disorder (e.g., sickle-cell disease) by administering to a subject in need thereof an effective amount of a compound of Formula (I′):

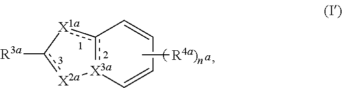

or a tautomer thereof, or a pharmaceutically acceptable salt of the compound or the tautomer, wherein
X^{1a} is O, S, CR^{1a}R^{11a}, or NR^{1a′} when [symbol] is a single bond, or X^{1a} is N when [symbol] is a double bond;
X^{2a} is N or CR^{2a} when [symbol] is a double bond, or X^{2a} is NR^{2a′} when [symbol] is a single bond;
X^{3a} is N or C; when X^{3a} is N, [symbol] is a double bond and [symbol] is a single bond, and when X^{3a} s is C, [symbol] a is a single bond and [symbol] is double bond;
each of R^{1a}, R^{2a} and R^{11a}, independently, is -Q^{1a}-T^{1a}, in which each Q^{1a} independently is a bond or C₁-C₆ alkylene, C₂-C₆ alkenylene, or C₂-C₆ alkynylene linker optionally substituted with one or more of halo, cyano, hydroxyl, or C₁-C₆ alkoxyl, and each Tia independently is H, halo, cyano, NR^{5a}R^{6a}, C(O)NR^{5a}R^{6a}, —OC(O)NR^{5a}R^{6a}, C(O)OR^{5a}, —OC(O)R^{5a}, C(O)R^{5a}, —NR^{5a}C(O)R^{6a}, —NR^{5a}C(O)OR^{6a}, OR^{5a}, or R^{S1a}, in which R^{S1a} is C₃-C₁₂ cycloalkyl, phenyl, 4- to 12-membered heterocycloalkyl containing 1-4 heteroatoms selected from N, O, and S, or a 5- or 6-membered heteroaryl and R^{S1a} is optionally substituted with one or more of halo, C₁-C₆ alkyl, hydroxyl, oxo, —C(O)R^{6a}, —SO₂R^{5a}, —SO₂N(R^{5a})₂, —NR^{5a}C(O)R^{6a}, amino, mono- or di-alkylamino, or C₁-C₆ alkoxyl; or
R^{1a} and R^{11a} together with the carbon atom to which they are attached form a C₃-C₁₂ cycloalkyl or 4- to 12-membered heterocycloalkyl containing 1-4 heteroatoms selected from N, O, and S, wherein the C₃-C₁₂ cycloalkyl or 4- to 12-membered heterocycloalkyl is optionally substituted with one or more of halo, C₁-C₆ alkyl, hydroxyl, oxo, amino, mono- or di-alkylamino, or C₁-C₆ alkoxyl;
each of R^{1a′} and R^{2a′}, independently, is -Q^{2a}-T^{2a}, in which Q^{2a} is a bond or C₁-C₆ alkylene, C₂-C₆ alkenylene, or C₂-C₆ alkynylene linker optionally substituted with one or more of halo, cyano, hydroxyl, or C₁-C₆ alkoxyl, and T^{2a} is H, halo, cyano, or R^{S2a}, in which R^{S2a} is C₃-C₁₂ cycloalkyl, phenyl, 4- to 12-membered heterocycloalkyl containing 1-4 heteroatoms selected from N, O, and S, or a 5- or 6-membered heteroaryl and R^{S2a} is optionally substituted with one or more of halo, C₁-C₆ alkyl, hydroxyl, oxo, —C(O)R^{6a}, —SO₂R^{5a}, —SO₂N(R^{5a})₂, —NR^{5a}C(O)R^{6a}, amino, mono- or di-alkylamino, or C₁-C₆ alkoxyl;
R^{3a} is H, NR^{aa}R^{ba}, OR^{aa}, or R^{S4a}, in which R^{S4a} is C₁-C₆ alkyl, C₂-C₆ alkenyl, C₂-C₆ alkynyl, C₃-C₁₂ cycloalkyl, phenyl, 5- or 6-membered heteroaryl, or 4- to 12-membered heterocycloalkyl containing 1-4 heteroatoms selected from N, O, and S, wherein each of R^{aa} and R^{ba} independently is H or R^{S5a}, or R^{aa} and R^{ba} together with the nitrogen atom to which they are attached form a 4- to 12-membered heterocycloalkyl containing 1-4 heteroatoms selected from N, O, and S; in which R^{S5a} is C₁-C₆ alkyl, phenyl, 5- or 6-membered heteroaryl, or 4- to 12-membered heterocycloalkyl containing 1-4 heteroatoms selected from N, O, and S, and each of R^{S4a}, R^{S5a}, and the heterocycloalkyl formed by R^{aa} and R^{ba} is independently optionally substituted with one or more of halo, hydroxyl, oxo, CN, amino, mono- or di-alkylamino, C₁-C₆ alkyl, C₁-C₆ alkoxyl, C₃-C₁₂ cycloalkyl, phenyl, 5- or 6-membered heteroaryl, or 4- to 12-membered heterocycloalkyl containing 1-4 heteroatoms selected from N, O, and S, or alternatively;
R^{3a} and one of R^{1a′}, R^{2a′}, R^{1a}, R^{2a} and R^{11a}, together with the atoms to which they are attached, form a 5- or 6-membered heteroaryl that is optionally substituted with one or more of halo, C₁-C₃ alkyl, hydroxyl or C₁-C₃ alkoxyl; or
R^{3a} is oxo and [symbol] is a single bond;
each R^{4a} independently is -Q^{3a}-T^{3a}, in which each Q^{3a} independently is a bond or C₁-C₆ alkylene, C₂-C₆ alkenylene, or C₂-C₆ alkynylene linker optionally substituted with one or more of halo, cyano, hydroxyl, amino, mono- or di-alkylamino, or C₁-C₆ alkoxyl, and each T^{3a} independently is H, halo, cyano, OR^{7a}, OR^{8a}, C(O)R^{8a}, NR^{7a}R^{8a}, C(O)NR^{7a}R^{8a}, NR^{7a}C(O)R^{8a}, C₆-C₁₀ aryl, 5- to 10-membered heteroaryl, C₃-C₁₂ cycloalkyl, or 4- to 12-membered heterocycloalkyl containing 1-4 heteroatoms selected from N, O, and S, and wherein the C₆-C₁₀ aryl, 5- to 10-membered heteroaryl, C₃-C₁₂ cycloalkyl or 4- to 12-membered heterocycloalkyl is optionally substituted with one or more of halo, hydroxyl, cyano, C₁-C₆ haloalkyl, —SO₂R^{5a}, C₁-C₆ alkoxyl or C₁-C₆ alkyl optionally substituted with one or more of NR^{5a}R^{6a};
each of R^{5a}, R^{6a}, and R^{7a}, independently, is H or C₁-C₆ alkyl optionally substituted with one or more of halo, cyano, hydroxyl, amino, mono- or di-alkylamino, or C₁-C₆ alkoxyl;
R^{8a} is -Q^{4a}-T^{4a}, in which Q^{4a} is a bond or C₁-C₆ alkylene, C₂-C₆ alkenylene, or C₂-C₆ alkynylene linker optionally substituted with one or more of halo, cyano, hydroxyl, or C₁-C₆ alkoxyl, and T^{4a} is H, halo, or R^{S3a}, in which R^{S3a} is C₃-C₁₂ cycloalkyl, C₆-C₁₀ aryl, 4- to 12-membered heterocycloalkyl containing 1-4 heteroatoms selected from N, O and S, or a 5- to 10-membered heteroaryl, and R^{S3a} is optionally substituted with one or more -Q^{5a}T^{5a}, wherein each Q^{5a} independently is a bond or C₁-C₃ alkylene, C₂-C₃ alkenylene, or C₂-C₃ alkynylene linker each optionally substituted with one or more of halo, cyano, hydroxyl, or C₁-C₆ alkoxy, and each TV independently is selected from the group consisting of H, halo, cyano, C₁-C₆ alkyl, C₃-C₁₂ cycloalkyl, C₆-C₁₀ aryl, 4- to 7-membered heterocycloalkyl containing 1-4 heteroatoms selected from N, O, and S, 5- to 6-membered heteroaryl, OR^{ca}, C(O)R^{ca}, NR^{ca}R^{da}, C(O)NR^{ca}R^{da}, S(O)₂R^{ca}, and NR^{ca}C(O)R^{da}, each of R^{ca} and R^{da} independently being H or C₁-C₆ alkyl optionally substituted with one or more halo; or -Q^{5a}-T^{5a} is oxo; and
n is 1, 2, 3, or 4.

In some embodiments, the compound is not

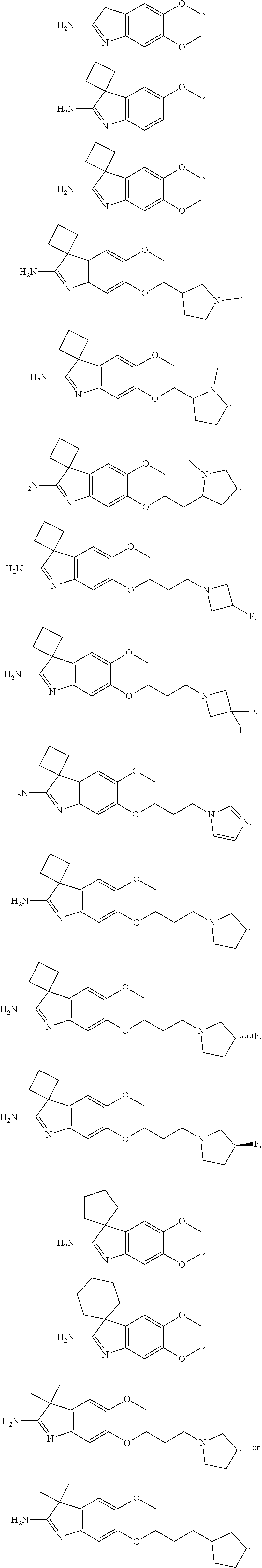

In some embodiments, when n is 2, X^{1a} is CR^{1a}R^{11a}, X^{2a} is N, X^{3a} is C, R^{3a} is NH₂, and at least one R^{4a} is OR^{7a}, then one of (1)-(4) below applies:
(1) at least one of R^{1a} and R^{11a} is -Q^{1a}-T^{1a}, in which Q^{1a} is a C₁-C₆ alkylene linker optionally substituted with one or more of halo, cyano, hydroxyl, or C₁-C₆ alkoxyl, and T^{1a} is cyano, NR^{5a}R^{6a}, C(O)NR^{5a}R^{6a}, —OC(O)NR^{5a}R^{6a}, C(O)OR^{5a}, —OC(O)R^{5a}, C(O)R^{5a}, —NR^{5a}C(O)R^{6a}, —NR^{5a}C(O)OR^{6a}, OR^{5a}, or R^{S1a}, in which R^{S1a} is C₃-C₁₂ cycloalkyl, phenyl, 4- to 12-membered heterocycloalkyl containing 1-4 heteroatoms selected from N, O, and S, or a 5- or 6-membered heteroaryl and R^{S1a} is optionally substituted with one or more of halo, C₁-C₆ alkyl, hydroxyl, oxo, —C(O)R^{6a}, —SO₂R^{5a}, —SO₂N(R^{5a})₂, —NR^{5a}C(O)R^{6a}, amino, mono- or di-alkylamino, or C₁-C₆ alkoxyl; or
(2) at least one of R^{1a} and R^{11a} is -Q^{1a}-T^{1a}, in which Q^{1a} is a C₂-C₆ alkenylene or C₂-C₆ alkynylene linker optionally substituted with one or more of halo, cyano, hydroxyl, or C₁-C₆ alkoxyl, and T^{1a} is H, halo, cyano, NR^{5a}R^{6a} C(O)NR^{5a}R^{6a}, —OC(O)NR^{5a}R^{6a}, C(O)OR^{5a}, —OC(O)R^{5a}, C(O)R^{5a}, —NR^{5a}C(O)R^{6a}, —NR^{5a}C(O)OR^{6a}, OR^{5a}, or R^{S1a}, in which R^{S1a} is C₃-C₁₂ cycloalkyl, phenyl, 4- to 12-membered heterocycloalkyl containing 1-4 heteroatoms selected from N, O, and S, or a 5- or 6-membered heteroaryl and R^{S1a} is optionally substituted with one or more of halo, C₁-C₆ alkyl, hydroxyl, oxo, —C(O)R^{6a}, —SO₂R^{5a}, —SO₂N(R^{5a})₂, —NR^{5a}C(O)R^{6a}, amino, mono- or di-alkylamino, or C₁-C₆ alkoxyl; or
(3) at least one of R^{1a} and R^{11a} is -Q^{1a}-T^{1a}, in which Q^{1a} is a bond, and T^{1a} is halo, cyano, NR^{5a}R^{6a}, C(O)NR^{5a}R^{6a}, —OC(O)NR^{5a}R^{6a}, C(O)OR^{5a}, —OC(O)R^{5a}, C(O)R^{5a}, —NR^{5a}C(O)R^{6a}, —NR^{5a}C(O)OR^{6a}, OR^{5a}, or R^{S1a}, in which R^{S1a} is C₃-C₁₂ cycloalkyl, phenyl, 4- to 12-membered heterocycloalkyl containing 1-4 heteroatoms selected from N, O, and S, or a 5- or 6-membered heteroaryl and R^{S1a} is optionally substituted with one or more of halo, C₁-C₆ alkyl, hydroxyl, oxo, —C(O)R^{6a}, —SO₂R^{5a}, —SO₂N(R^{5a})₂, —NR^{5a}C(O)R^{6a}, amino, mono- or di-alkylamino, or C₁-C₆ alkoxyl; or
(4) R^{1a} and R^{11a} together with the carbon atom to which they are attached form a C₇-C₁₂ cycloalkyl or 4- to 12-membered heterocycloalkyl containing 1-4 heteroatoms selected from N, O, and S, wherein the C₇-C₁₂ cycloalkyl or 4- to 12-membered heterocycloalkyl is optionally substituted with one or more of halo, C₁-C₆ alkyl, hydroxyl, oxo, amino, mono- or di-alkylamino, or C₁-C₆ alkoxyl.

In some embodiments, at least one of X^{2a} and X^{3a} is N.

In some embodiments, at least two of X^{1a}, X^{2a}, and X^{3a} comprise N.

In some embodiments, at least one of [symbol] and [symbol] is a double bond.

In some embodiments, [symbol] a double bond.

In some embodiments, [symbol] is a single bond.

In some embodiments, X^{2a} is NR^{2a′} and R^{3a} is oxo.

In some embodiments, X^{2a} is N and X^{3a} is C.

In some embodiments, X^{2a} is CR^{2a} and X^{3a} is N.

In some embodiments, X^{1a} is S.

In some embodiments, X^{1a} is NR^{1a′}.

In some embodiments, X^{1a} is CR^{1a}R^{11a}.

In some embodiments, R^{1a} and R^{11a} together with the carbon atom to which they are attached form a 4- to 7-membered heterocycloalkyl containing 1-4 heteroatoms selected from N, O, and S, wherein the 4- to 7-membered heterocycloalkyl is optionally substituted with one or more of halo, C₁-C₆ alkyl, hydroxyl, oxo, amino, mono- or di-alkylamino, or C₁-C₆ alkoxyl.

In some embodiments, n is 1 or 2.

In some embodiments, n is 2.

In some embodiments, the compound is of Formula (IIa′), (IIb′), (IIc′), (IId′), (IIe′), (IIIa′), (IIIb′), (IIIc′), (IIId′), (IIIe′), (IIIf′), (IVa′), or (IVb′):

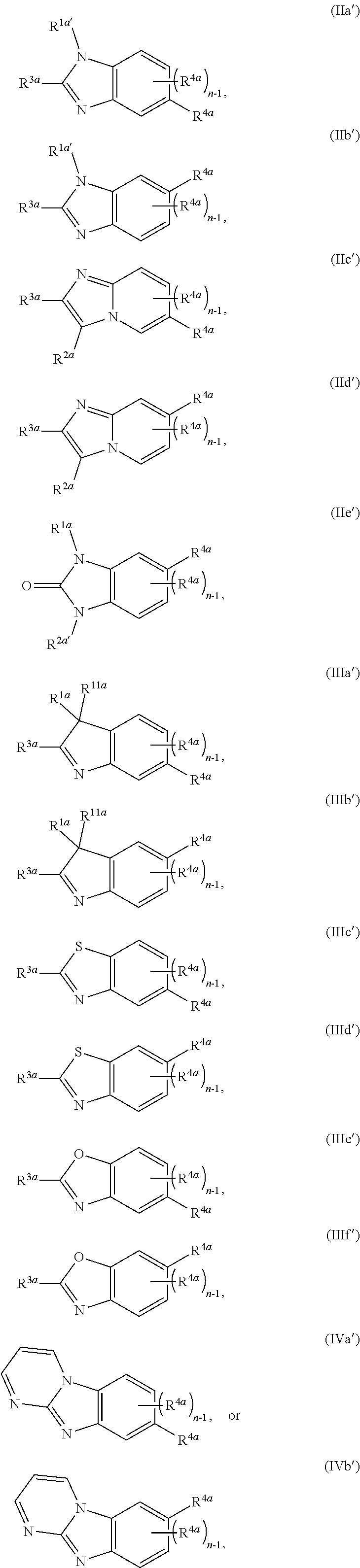

a tautomer thereof, or a pharmaceutically acceptable salt of the compound or the tautomer.

In some embodiments, the compound is of Formula (IIf′), (IIg′), (IIh′), (IIIi′), (IIIj′), (IIIk′), or (IIIl′):

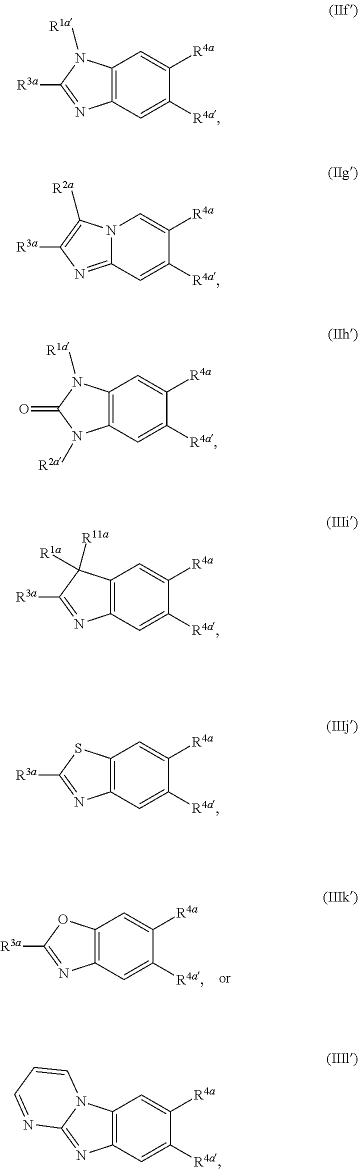

a tautomer thereof, or a pharmaceutically acceptable salt of the compound or the tautomer, wherein
R^{3a} is H, NR^{aa}R^{ba}, OR^{aa}, or R^{S4a}, in which R^{S4a} is C₁-C₆ alkyl, C₂-C₆ alkenyl, C₂-C₆ alkynyl, C₃-C₁₂ cycloalkyl, phenyl, 5- or 6-membered heteroaryl, or 4- to 12-membered heterocycloalkyl containing 1-4 heteroatoms selected from N, O, and S, wherein each of R^{ca} and R^{ba} independently is H or R^{S5a}, or R^{aa} and R^{ba} together with the nitrogen atom to which they are attached form a 4- to 12-membered heterocycloalkyl containing 1-4 heteroatoms selected from N, O, and S; in which R^{S5a} is C₁-C₆ alkyl, phenyl, 5- or 6-membered heteroaryl, or 4- to 12-membered heterocycloalkyl containing 1-4 heteroatoms selected from N, O, and S, and each of R^{S4a}, R^{S5a}, and the heterocycloalkyl formed by R^{aa} and R^{ba} is independently optionally substituted with one or more of halo, hydroxyl, oxo, CN, amino, mono- or di-alkylamino, C₁-C₆ alkyl, C₁-C₆ alkoxyl, C₃-C₁₂ cycloalkyl, phenyl, 5- or 6-membered heteroaryl, or 4- to 12-membered heterocycloalkyl containing 1-4 heteroatoms selected from N, O, and S;
each of R^{4a} and R^{4a′} independently is -Q^{3a}-T^{3a}, in which each Q^{3a} independently is a bond or C₁-C₆ alkylene, C₂-C₆ alkenylene, or C₂-C₆ alkynylene linker optionally substituted with one or more of halo, cyano, hydroxyl, amino, mono- or di-alkylamino, or C₁-C₆ alkoxyl, and each T³a independently is H, halo, cyano, OR^{7a}, OR^{8a}, C(O)R^{8a}, NR^{7a}R^{8a}, C(O)NR^{7a}R^{8a}, NR^{7a}C(O)R^{8a}, C₆-C₁₀ aryl, 5- to 10-membered heteroaryl, C₃-C₁₂ cycloalkyl, or 4- to 12-membered heterocycloalkyl containing 1-4 heteroatoms selected from N, O, and S, and wherein the C₆-C₁₀ aryl, 5- to 10-membered heteroaryl, C₃-C₁₂ cycloalkyl or 4- to 12-membered heterocycloalkyl is optionally substituted with one or more of halo, hydroxyl, cyano, C₁-C₆ haloalkyl, —SO₂R^{5a}, C₁-C₆ alkoxyl or C₁-C₆ alkyl optionally substituted with one or more of NR^{5a}R^{6a};
each of R^{5a}, R^{6a}, and R^{7a}, independently, is H or C₁-C₆ alkyl optionally substituted with one or more of halo, cyano, hydroxyl, amino, mono- or di-alkylamino, or C₁-C₆ alkoxyl;
R^{8a} is -Q^{4a}-T^{4a}, in which Q^{4a} is a bond or C₁-C₆ alkylene, C₂-C₆ alkenylene, or C₂-C₆ alkynylene linker optionally substituted with one or more of halo, cyano, hydroxyl, or C₁-C₆ alkoxyl, and T^{4a} is H, halo, or R^{S3a}, in which R^{S3a} is C₃-C₁₂ cycloalkyl, C₆-C₁₀ aryl, 4- to 12-membered heterocycloalkyl containing 1-4 heteroatoms selected from N, O and S, or a 5- to 10-membered heteroaryl, and R^{S3a} is optionally substituted with one or more -Q^{5a}-T^{5a}, wherein each Q^{5a} independently is a bond or C₁-C₃ alkylene, C₂-C₃ alkenylene, or C₂-C₃ alkynylene linker each optionally substituted with one or more of halo, cyano, hydroxyl, or C₁-C₆ alkoxy, and each T^{5a} independently is selected from the group consisting of H, halo, cyano, C₁-C₆ alkyl, C₃-C₁₂ cycloalkyl, C₆-C₁₀ aryl, 4- to 7-membered heterocycloalkyl containing 1-4 heteroatoms selected from N, O, and S, 5- to 6-membered heteroaryl, OR^{ca}, C(O)R^{ca}, NR^{ca}, C(O)NR^{ca}R^{da}, S(O)₂R^{ca}, and NR^{ca}C(O)R^{da}, each of R^{ca} and R^{da} independently being H or C₁-C₆ alkyl optionally substituted with one or more halo; or -Q^{5a}-T^{5a} is oxo.

In some embodiments, the compound is not one of those described in EP 0356234; U.S. Pat. Nos. 5,106,862; 6,025,379; 9,284,272; WO2002/059088; and/or WO2015/200329.

In some embodiments, when n is 2, X^{1a} is CR^{1a}R^{11a}, X^{2a} is N, X^{3a} is C, R^{3a} is NH₂, and at least one R^{4a} is OR^{7a}, then at least one of R^{1a} and R^{11a} is -Q^{1a}-T^{1a}, in which Q^{1a} is a C₁-C₆ alkylene linker optionally substituted with one or more of halo, cyano, hydroxyl, or C₁-C₆ alkoxyl, and T^{1a} is cyano, NR^{5a}R^{6a}, C(O)NR^{5a}R^{6a}, —OC(O)NR^{5a}R^{6a}, C(O)OR^{5a}, —OC(O)R^{5a}, C(O)R^{5a}, —NR^{5a}C(O)R^{6a}, —NR^{5a}C(O)OR^{6a}, OR^{5a}, or R^{S1a}, in which R^{S1a} is C₃-C₁₂ cycloalkyl, phenyl, 4- to 12-membered heterocycloalkyl (e.g., 4- to 7-membered heterocycloalkyl) containing 1-4 heteroatoms selected from N, O, and S, or a 5- or 6-membered heteroaryl and R^{S1a} is optionally substituted with one or more of halo, C₁-C₆ alkyl, hydroxyl, oxo, —C(O)R^{6a}, —SO₂R^{5a}, —SO₂N(R^{5a})₂, —NR^{5a}C(O)R^{6a}, amino, mono- or di-alkylamino, or C₁-C₆ alkoxyl.

In some embodiments, when n is 2, X^{1a} is CR^{1a}R^{11a}, X^{2a} is N, X^{3a} is C, R^{3a} is NH₂, and at least one R^{4a} is OR^{7a}, then at least one of R^{1a} and R^{11a} is -Q^{1a}-T^{1a}, in which Q^{1a} is a C₂-C₆ alkenylene or C₂-C₆ alkynylene linker optionally substituted with one or more of halo, cyano, hydroxyl, or C₁-C₆ alkoxyl, and T^{1a} is H, halo, cyano, NR^{5a}R^{6a}, C(O)NR^{5a}R^{6a}, —OC(O)NR^{5a}R^{6a}, C(O)OR^{5a}, —OC(O)R^{5a}, C(O)R^{5a}, —NR^{5a}C(O)R^{6a}, —NR^{5a}C(O)OR^{6a}, OR^{5a}, or R^{S1a}, in which R^{S1a} is C₃-C₁₂ cycloalkyl, phenyl, 4- to 12-membered heterocycloalkyl (e.g., 4- to 7-membered heterocycloalkyl) containing 1-4 heteroatoms selected from N, O, and S, or a 5- or 6-membered heteroaryl and R^{S1a} is optionally substituted with one or more of halo, C₁-C₆ alkyl, hydroxyl, oxo, —C(O)R^{6a}, —SO₂R^{5a}, —SO₂N(R^{5a})₂, —NR^{5a}C(O)R^{6a}, amino, mono- or di-alkylamino, or C₁-C₆ alkoxyl.

In some embodiments, when n is 2, X^{1a} is CR^{1a}R^{11a}, X^{2a} is N, X^{3a} is C, R^{3a} is NH₂, and at least one R^{4a} is OR^{7a}, then at least one of R^{1a} and R^{11a} is -Q^{1a}-T^{1a}, in which Q^{1a} is a bond, and T^{1a} is halo, cyano, NR^{5a}R^{6a}, C(O)NR^{5a}R^{6a}, —OC(O)NR^{5a}R^{6a}, C(O)OR^{5a}, —OC(O)R^{5a}, C(O)R^{5a}, —NR^{5a}C(O)R^{6a}, —NR^{5a}C(O)OR^{6a}, OR^{5a}, or R^{S1a}, in which R^{S1a} is C₃-C₁₂ cycloalkyl, phenyl, 4- to 12-membered heterocycloalkyl (e.g., 4- to 7-membered heterocycloalkyl) containing 1-4 heteroatoms selected from N, O, and S, or a 5- or 6-membered heteroaryl and R^{S1a} is optionally substituted with one or more of halo, C₁-C₆ alkyl, hydroxyl, oxo, —C(O)R^{6a}, —SO₂R^{5a}, —SO₂N(R^{5a})₂, —NR^{5a}C(O)R^{6a}, amino, mono- or di-alkylamino, or C₁-C₆ alkoxyl.

In some embodiments, when n is 2, X^{1a} is CR^{1a}R^{11a}, X^{2a} is N, X^{3a} is C, R^{3a} is NH₂, and at least one R^{4a} is OR^{7a}, then R^{1a} and R^{11a} together with the carbon atom to which they are attached form a C₇-C₁₂ cycloalkyl or 4- to 12-membered heterocycloalkyl (e.g., 4- to 7-membered heterocycloalkyl) containing 1-4 heteroatoms selected from N, O, and S, wherein the C₇-C₁₂ cycloalkyl or 4- to 12-membered heterocycloalkyl (e.g., 4- to 7-membered heterocycloalkyl) is optionally substituted with one or more of halo, C₁-C₆ alkyl, hydroxyl, oxo, amino, mono- or di-alkylamino, or C₁-C₆ alkoxyl.

In some embodiments, R^{2a} is -Q^{1a}-T^{1a}, in which Q^{1a} is a bond or C₁-C₆ alkylene, C₂-C₆ alkenylene, or C₂-C₆ alkynylene linker optionally substituted with one or more of halo, cyano, hydroxyl, or C₁-C₆ alkoxyl, and Tia is H, halo, cyano, or R^{S1a}, in which R^{S1a} is C₃-C₁₂ cycloalkyl (e.g., C₃-C₈ cycloalkyl), phenyl, 4- to 12-membered heterocycloalkyl (e.g., 4- to 7-membered heterocycloalkyl) containing 1-4 heteroatoms selected from N, O, and S, or a 5- or 6-membered heteroaryl and R^{S1a} is optionally substituted with one or more of halo, C₁-C₆ alkyl, hydroxyl, oxo, amino, mono- or di-alkylamino, or C₁-C₆ alkoxyl.

In some embodiments, R^{2a} is C₁-C₆ alkyl optionally substituted with one or more of halo, cyano, hydroxyl, or C₁-C₆ alkoxyl. In some embodiments, R^{2a} is unsubstituted C₁-C₆ alkyl.

In some embodiments, Q^{1a} is a bond or C₁-C₆ alkylene linker optionally substituted with one or more of halo, cyano, hydroxyl, or C₁-C₆ alkoxyl, and Tia is H, halo, cyano, or R^{S1a}, in which R^{S1a} is C₃-C₁₂ cycloalkyl (e.g., C₃-C₈ cycloalkyl), phenyl, 4- to 12-membered heterocycloalkyl (e.g., 4- to 7-membered heterocycloalkyl) containing 1-4 heteroatoms selected from N, O, and S, or a 5- or 6-membered heteroaryl and R^{S1a} is optionally substituted with one or more of halo, C₁-C₆ alkyl, hydroxyl, oxo, amino, mono- or di-alkylamino, or C₁-C₆ alkoxyl.

In some embodiments, Q^{1a} is a C₂-C₆ alkenylene or C₂-C₆ alkynylene linker optionally substituted with one or more of halo, cyano, hydroxyl, or C₁-C₆ alkoxyl, and Tia is H, halo, cyano, or R^{S1a}, in which R^{S1a} is C₃-C₁₂ cycloalkyl (e.g., C₃-C₈ cycloalkyl), phenyl, 4- to 12-membered heterocycloalkyl (e.g., 4- to 7-membered heterocycloalkyl) containing 1-4 heteroatoms selected from N, O, and S, or a 5- or 6-membered heteroaryl and R^{S1a} is optionally substituted with one or more of halo, C₁-C₆ alkyl, hydroxyl, oxo, amino, mono- or di-alkylamino, or C₁-C₆ alkoxyl.

In some embodiments, R^{1a′} is -Q^{2a}-T^{2a}, in which Q^{2a} is a bond or C₁-C₆ alkylene, C₂-C₆ alkenylene, or C₂-C₆ alkynylene linker optionally substituted with one or more of halo, cyano, hydroxyl, or C₁-C₆ alkoxyl, and T²a is H, halo, cyano, or R^{S2a}, in which R^{S2a} is C₃-C₁₂ cycloalkyl (e.g., C₃-C₈ cycloalkyl), phenyl, 4- to 12-membered heterocycloalkyl (e.g., 4- to 7-membered heterocycloalkyl) containing 1-4 heteroatoms selected from N, O, and S, or a 5- or 6-membered heteroaryl and R^{S2a} is optionally substituted with one or more of halo, C₁-C₆ alkyl, hydroxyl, oxo, amino, mono- or di-alkylamino, or C₁-C₆ alkoxyl.

In some embodiments, R^{2a′} is -Q^{2a}-T^{2a}, in which Q^{2a} is a bond or C₁-C₆ alkylene, C₂-C₆ alkenylene, or C₂-C₆ alkynylene linker optionally substituted with one or more of halo, cyano, hydroxyl, or C₁-C₆ alkoxyl, and T^{2a}, is H, halo, cyano, or R^{S2a}, in which R^{S2a} is C₃-C₁₂ cycloalkyl (e.g., C₃-C₈ cycloalkyl), phenyl, 4- to 12-membered heterocycloalkyl (e.g., 4- to 7-membered heterocycloalkyl) containing 1-4 heteroatoms selected from N, O, and S, or a 5- or 6-membered heteroaryl and R^{S2a} is optionally substituted with one or more of halo, C₁-C₆ alkyl, hydroxyl, oxo, amino, mono- or di-alkylamino, or C₁-C₆ alkoxyl.

In some embodiments, each Q^{2a} independently is a bond or C₁-C₆ alkylene linker optionally substituted with one or more of halo and each T^{2a} independently is H, halo, C₃-C₁₂ cycloalkyl (e.g., C₃-C₈ cycloalkyl), or a 4- to 7-membered heterocycloalkyl.

In some embodiments, each Q^{2a} independently is C₂-C₆ alkenylene or C₂-C₆ alkynylene linker optionally substituted with one or more of halo, cyano, hydroxyl, or C₁-C₆ alkoxyl.

In some embodiments, R^{2a′} is H or C₁-C₆ alkyl.

In some embodiments, R^{3a} is H.

In some embodiments, R^{3a} is NR^{aa}R^{ba} or OR^{aa}, wherein each of R^{aa} and R^{ba} independently is H or C₁-C₆ alkyl optionally substituted with one or more of halo, hydroxyl, CN, amino, mono- or di-alkylamino, or C₁-C₆ alkoxyl.

In some embodiments, R^{3a} is NR^{aa}R^{ba} or OR^{aa}, wherein each of R^{aa} and R^{ba} independently is H or C₁-C₆ alkyl optionally substituted with one or more of halo, hydroxyl, amino, mono- or di-alkylamino, C₁-C₆ alkoxyl, C₃-C₁₂ cycloalkyl, phenyl, 5- or 6-membered heteroaryl, or 4- to 12-membered heterocycloalkyl (e.g., 4- to 7-membered heterocycloalkyl) containing 1-4 heteroatoms selected from N, O, and S.

In some embodiments, R^{3a} is NR^{aa}R^{ba}.

In some embodiments, each of R^{aa} and R^{ba} independently is H or R^{S5a}.

In some embodiments, one of R^{aa} and R^{ba} is H and the other is R^{S5a}.

In some embodiments, R^{aa} and R^{ba} together with the nitrogen atom to which they are attached form a 4- to 12-membered heterocycloalkyl (e.g., 4- to 7-membered heterocycloalkyl), which is optionally substituted with one or more of halo, hydroxyl, oxo, CN, amino, mono- or di-alkylamino, C₁-C₆ alkyl, C₁-C₆ alkoxyl, C₃-C₁₂ cycloalkyl, phenyl, 5- or 6-membered heteroaryl, or 4- to 12-membered heterocycloalkyl (e.g., 4- to 7-membered heterocycloalkyl).

In some embodiments, R^{aa} and R^{ba} together with the nitrogen atom to which they are attached form a 4- to 12-membered heterocycloalkyl (e.g., 4- to 7-membered heterocycloalkyl), which is optionally substituted with one or more of halo, hydroxyl, oxo, CN, amino, mono- or di-alkylamino, C₁-C₆ alkyl, or C₁-C₆ alkoxyl.

In some embodiments, R^{S5a} is C₁-C₆ alkyl, and R^{S5a} is optionally substituted with one or more of halo, hydroxyl, CN, amino, mono- or di-alkylamino, C₁-C₆ alkoxyl, C₃-C₁₂ cycloalkyl, phenyl, 5- or 6-membered heteroaryl, or 4- to 12-membered heterocycloalkyl (e.g., 4- to 7-membered heterocycloalkyl).

In some embodiments, R^{S5a} is phenyl, 5- or 6-membered heteroaryl, or 4- to 12-membered heterocycloalkyl (e.g., 4- to 7-membered heterocycloalkyl), and R^{S5a} is optionally substituted with one or more of halo, hydroxyl, oxo, CN, amino, mono- or di-alkylamino, C₁-C₆ alkyl, C₁-C₆ alkoxyl, C₃-C₁₂ cycloalkyl, phenyl, 5- or 6-membered heteroaryl, or 4- to 12-membered heterocycloalkyl (e.g., 4- to 7-membered heterocycloalkyl).

In some embodiments, the compound is of Formulae (Va′), (Vb′), (Vc′), (Vd′), (Ve′), or (Vf′):

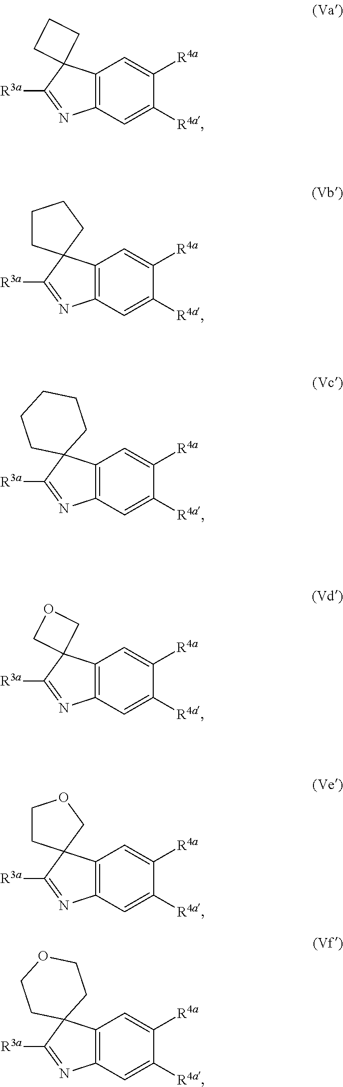

a tautomer thereof, or a pharmaceutically acceptable salt of the compound or the tautomer, wherein
R^{3a} is H, NR^{aa}R^{ba}, OR^{aa}, or R^{S4a}, in which R^{S4a} is C₁-C₆ alkyl, C₂-C₆ alkenyl, C₂-C₆ alkynyl, C₃-C₁₂ cycloalkyl, phenyl, 5- or 6-membered heteroaryl, or 4- to 12-membered heterocycloalkyl containing 1-4 heteroatoms selected from N, O, and S, wherein each of R^{aa} and R^{ba} independently is H or R^{S5a}, or R^{aa} and R^{ba} together with the nitrogen atom to which they are attached form a 4- to 12-membered heterocycloalkyl containing 1-4 heteroatoms selected from N, O, and S; in which R^{S5a} is C₁-C₆ alkyl, phenyl, 5- or 6-membered heteroaryl, or 4- to 12-membered heterocycloalkyl containing 1-4 heteroatoms selected from N, O, and S, and each of R^{S4a}, R^{S5a}, and the heterocycloalkyl formed by R^{aa} and R^{ba} is independently optionally substituted with one or more of halo, hydroxyl, oxo, CN, amino, mono- or di-alkylamino, C₁-C₆ alkyl, C₁-C₆ alkoxyl, C₃-C₁₂ cycloalkyl, phenyl, 5- or 6-membered heteroaryl, or 4- to 12-membered heterocycloalkyl containing 1-4 heteroatoms selected from N, O, and S;
each of R^{4a} and R^{4a′} independently is -Q^{3a}-T^{3a}, in which each Q^{3a} independently is a bond or C₁-C₆ alkylene, C₂-C₆ alkenylene, or C₂-C₆ alkynylene linker optionally substituted with one or more of halo, cyano, hydroxyl, amino, mono- or di-alkylamino, or C₁-C₆ alkoxyl, and each T^{3a} independently is H, halo, cyano, OR^{7a}, OR^{8a}, C(O)R^{8a}, NR^{7a}R^{8a}, C(O)NR^{7a}R^{8a}, NR^{7a}C(O)R^{8a}, C₆-C₁₀ aryl, 5- to 10-membered heteroaryl, C₃-C₁₂ cycloalkyl, or 4- to 12-membered heterocycloalkyl containing 1-4 heteroatoms selected from N, O, and S, and wherein the C₆-C₁₀ aryl, 5- to 10-membered heteroaryl, C₃-C₁₂ cycloalkyl or 4- to 12-membered heterocycloalkyl is optionally substituted with one or more of halo, hydroxyl, cyano, C₁-C₆ haloalkyl, —SO₂R^{5a}, C₁-C₆ alkoxyl or C₁-C₆ alkyl optionally substituted with one or more of NR^{5a}R^{6a};
each of R^{5a}, R^{6a}, and R^{7a}, independently, is H or C₁-C₆ alkyl optionally substituted with one or more of halo, cyano, hydroxyl, amino, mono- or di-alkylamino, or C₁-C₆ alkoxyl; and
R^{8a} is -Q^{4a}-T^{4a}, in which Q^{4a} is a bond or C₁-C₆ alkylene, C₂-C₆ alkenylene, or C₂-C₆ alkynylene linker optionally substituted with one or more of halo, cyano, hydroxyl, or C₁-C₆ alkoxyl, and T^{4a} is H, halo, or R^{S3a}, in which R^{S3a} is C₃-C₁₂ cycloalkyl, C₆-C₁₀ aryl, 4- to 12-membered heterocycloalkyl containing 1-4 heteroatoms selected from N, O and S, or a 5- to 10-membered heteroaryl, and R^{S3a} is optionally substituted with one or more -Q^{5a}-T^{5a}, wherein each Q^{5a} independently is a bond or C₁-C₃ alkylene, C₂-C₃ alkenylene, or C₂-C₃ alkynylene linker each optionally substituted with one or more of halo, cyano, hydroxyl, or C₁-C₆ alkoxy, and each T^{5a} independently is selected from the group consisting of H, halo, cyano, C₁-C₆ alkyl, C₃-C₁₂ cycloalkyl, C₆-C₁₀ aryl, 4- to 7-membered heterocycloalkyl containing 1-4 heteroatoms selected from N, O, and S, 5- to 6-membered heteroaryl, OR^{ca}, C(O)R^{ca}, NR^{ca}R^{da}, C(O)NR^{ca}R^{da}, S(O)₂R^{ca}, and NR^{ca}C(O)R^{da}, each of R^{ca} and R^{da} independently being H or C₁-C₆ alkyl optionally substituted with one or more halo; or -Q^{5a}-T^{5a} is oxo.

In some embodiments, when R^{3a} is —NH₂, then R^{4a} is not —OCH₃.

In some embodiments, when R^{3a} is —NH₂, and R^{4a} is not —OCH₃, then R^{4a′} is not OR^{8a}.

In some embodiments, R^{3a} is C₁-C₆ alkyl, C₂-C₆ alkenyl, or C₂-C₆ alkynyl, each of which is optionally substituted with one or more of halo, hydroxyl, oxo, CN, amino, mono- or di-alkylamino, C₁-C₆ alkoxyl, C₃-C₁₂ cycloalkyl, phenyl, 5- or 6-membered heteroaryl, or 4- to 12-membered heterocycloalkyl (e.g., 4- to 7-membered heterocycloalkyl) containing 1-4 heteroatoms selected from N, O, and S; in which each of the C₃-C₂ cycloalkyl, phenyl, 5- or 6-membered heteroaryl, and 4- to 12-membered heterocycloalkyl (e.g., 4- to 7-membered heterocycloalkyl) is independently optionally substituted with one or more of halo, hydroxyl, oxo, CN, amino, mono- or di-alkylamino, C₁-C₆ alkyl, or C₁-C₆ alkoxyl.

In some embodiments, R^{3a} is C₃-C₁₂ cycloalkyl or 4- to 12-membered heterocycloalkyl (e.g., 4- to 7-membered heterocycloalkyl) containing 1-4 heteroatoms selected from N, O, and S, wherein each of the C₃-C₁₂ cycloalkyl and 4- to 12-membered heterocycloalkyl (e.g., 4- to 7-membered heterocycloalkyl) is independently optionally substituted with one or more of halo, hydroxyl, oxo, CN, amino, mono- or di-alkylamino, C₁-C₆ alkyl, or C₁-C₆ alkoxyl.

In some embodiments, R^{3a} is

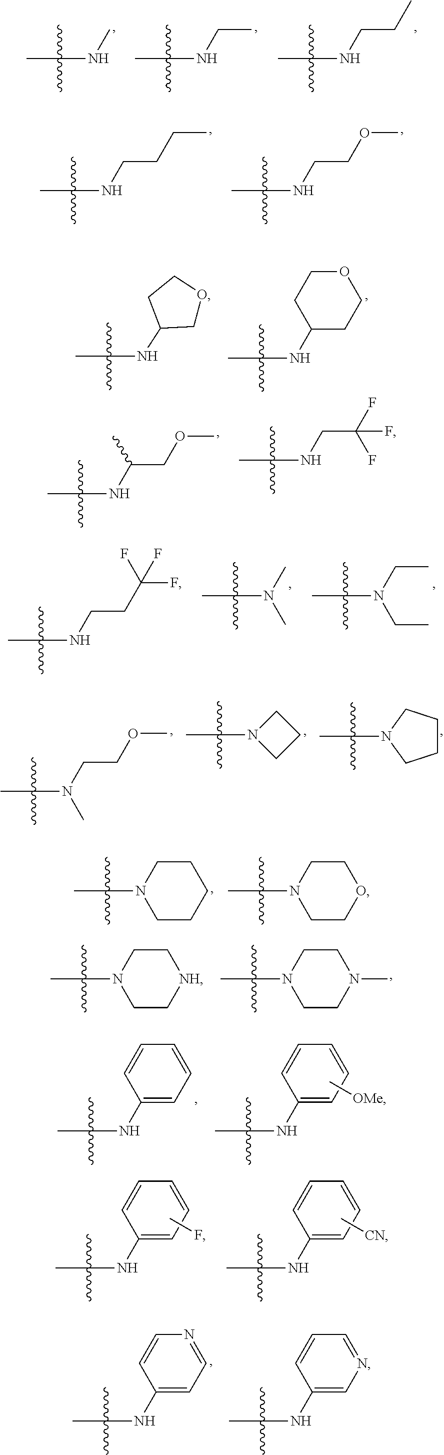

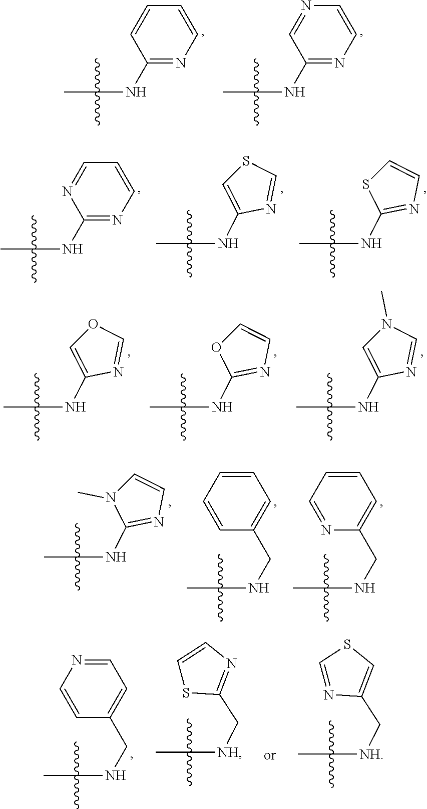

In some embodiments, R^{3a} is NH₂.

In some embodiments, R^{3a} is NR^{aa}R^{ba}, in which one of R^{aa} and R^{ba} is H and the other is C₁-C₆ alkyl optionally substituted with one or more of halo or C₁-C₆ alkoxyl.

In some embodiments, R^{3a} is oxo and [symbol] is a single bond.

In some embodiments, R^{3a} is OH.

In some embodiments, R^{3a} is C₁-C₆ alkoxyl.

In some embodiments, R^{3a} and one of R^{1a′}, R^{2a′}, R^{1a}, R^{2a} and R^{11a}, together with the atoms to which they are attached, form a 6-membered heteroaryl that is optionally substituted with one or more of halo, C₁-C₃ alkyl, hydroxyl or C₁-C₃ alkoxyl.

In some embodiments, R^{3a} and one of R^{1a′}, R^{2a′}, R^{1a}, R^{2a} and R^{11a}, together with the atoms to which they are attached, form a 5-membered heteroaryl that is optionally substituted with one or more of halo, C₁-C₃ alkyl, hydroxyl or C₁-C₃ alkoxyl.

In some embodiments, the compound is of Formulae (VIa′), (VIb′), (VIc′), (VId′), (VIe′), or (VIf′):

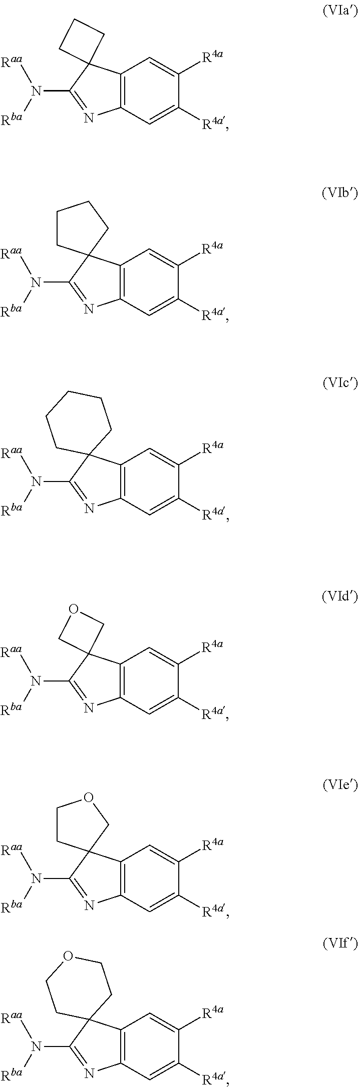

a tautomer thereof, or a pharmaceutically acceptable salt of the compound or the tautomer, wherein
each of R^{aa} and R^{ba} independently is H or R^{S5a}, or R^{aa} and R^{ba} together with the nitrogen atom to which they are attached form a 4- to 12-membered heterocycloalkyl containing 1-4 heteroatoms selected from N, O, and S; in which R^{S5a} is C₁-C₆ alkyl, phenyl, 5- or 6-membered heteroaryl, or 4- to 12-membered heterocycloalkyl containing 1-4 heteroatoms selected from N, O, and S, and each of R^{S4a}, R^{S5a}, and the heterocycloalkyl formed by R^{aa} and R^{ba} is independently optionally substituted with one or more of halo, hydroxyl, oxo, CN, amino, mono- or di-alkylamino, C₁-C₆ alkyl, C₁-C₆ alkoxyl, C₃-C₁₂ cycloalkyl, phenyl, 5- or 6-membered heteroaryl, or 4- to 12-membered heterocycloalkyl containing 1-4 heteroatoms selected from N, O, and S, or alternatively; and
each of R^{4a} and R^{4a′} independently is -Q^{3a}-T^{3a}, in which each Q^{3a} independently is a bond or C₁-C₆ alkylene, C₂-C₆ alkenylene, or C₂-C₆ alkynylene linker optionally substituted with one or more of halo, cyano, hydroxyl, amino, mono- or di-alkylamino, or C₁-C₆ alkoxyl, and each T^{3a} independently is H, halo, cyano, OR^{7a}, OR^{8a}, C(O)R^{8a}, NR^{7a}R^{8a}, C(O)NR^{7a}R^{8a}, NR^{7a}C(O)R^{8a}, C₆-C₁₀ aryl, 5- to 10-membered heteroaryl, C₃-C₁₂ cycloalkyl, or 4- to 12-membered heterocycloalkyl containing 1-4 heteroatoms selected from N, O, and S, and wherein the C₆-C₁₀ aryl, 5- to 10-membered heteroaryl, C₃-C₁₂ cycloalkyl or 4- to 12-membered heterocycloalkyl is optionally substituted with one or more of halo, hydroxyl, cyano, C₁-C₆ haloalkyl, —SO₂R^{5a}, C₁-C₆ alkoxyl or C₁-C₆ alkyl optionally substituted with one or more of NR^{5a}R^{6a};
each of R^{5a}, R^{6a}, and R^{7a}, independently, is H or C₁-C₆ alkyl optionally substituted with one or more of halo, cyano, hydroxyl, amino, mono- or di-alkylamino, or C₁-C₆ alkoxyl; and
R^{8a} is -Q^{4a}-T^{4a}, in which Q^{4a} is a bond or C₁-C₆ alkylene, C₂-C₆ alkenylene, or C₂-C₆ alkynylene linker optionally substituted with one or more of halo, cyano, hydroxyl, or C₁-C₆ alkoxyl, and T^{4a} is H, halo, or R^{S3a}, in which R^{S3a} is C₃-C₁₂ cycloalkyl, C₆-C₁₀ aryl, 4- to 12-membered heterocycloalkyl containing 1-4 heteroatoms selected from N, O and S, or a 5- to 10-membered heteroaryl, and R^{S3a} is optionally substituted with one or more -Q^{5a}-T^{5a}, wherein each Q^{5a} independently is a bond or C₁-C₃ alkylene, C₂-C₃ alkenylene, or C₂-C₃ alkynylene linker each optionally substituted with one or more of halo, cyano, hydroxyl, or C₁-C₆ alkoxy, and each T^{5a} independently is selected from the group consisting of H, halo, cyano, C₁-C₆ alkyl, C₃-C₁₂ cycloalkyl, C₆-C₁₀ aryl, 4- to 7-membered heterocycloalkyl containing 1-4 heteroatoms selected from N, O, and S, 5- to 6-membered heteroaryl, OR^{ca}, C(O)R^{ca}, NR^{ca}R^{da}, C(O)NR^{ca}R^{da}, S(O)₂R^{ca}, and NR^{ca}C(O)R^{da}, each of R^{ca} and R^{da} independently being H or C₁-C₆ alkyl optionally substituted with one or more halo; or -Q^{5a}-T^{5a} is oxo.

In some embodiments, at least one of R^{aa} and R^{ba} is R^{S5a}.

In some embodiments, when both of R^{aa} and R^{ba} are H, then R^{4a} is not —OCH₃.

In some embodiments, when both of R^{aa} and R^{ba} are H, and R^{4a} is —OCH₃, then R^{4a} is not OR^{8a}.

In some embodiments, each of R^{4a} and R^{4a′} is independently -Q^{3a}-T^{3a}, in which each Q^{3a} independently is a bond or C₁-C₆ alkylene, C₂-C₆ alkenylene, or C₂-C₆ alkynylene linker optionally substituted with one or more of halo, cyano, hydroxyl, amino, mono- or di-alkylamino, or C₁-C₆ alkoxyl, and each T^{3a} independently is H, halo, OR^{7a}, OR^{8a}, NR^{7a}R^{8a}, C₆-C₁₀ aryl, 5- to 10-membered heteroaryl, C₃-C₁₂ cycloalkyl, or 4- to 12-membered heterocycloalkyl.

In some embodiments, R^{4a′} is -Q^{3a}-T^{3a}, in which Q^{3a} is a bond or C₁-C₆ alkylene linker, and T^{3a} is H, halo, OR^{7a}, C₆-C₁₀ aryl, or 5- to 10-membered heteroaryl.

In some embodiments, R^{4′} is -Q^{3a}-T^{3a}, in which Q^{3a} independently is a bond or C₁-C₆ alkylene, C₂-C₆ alkenylene, or C₂-C₆ alkynylene linker optionally substituted with one or more of halo, cyano, hydroxyl, amino, mono- or di-alkylamino, or C₁-C₆ alkoxyl, and each T^{3a} independently is H, OR^{7a}, OR^{8a}, NR^{7a}R^{8a}, C₃-C₁₂ cycloalkyl, or 4- to 12-membered heterocycloalkyl.

In some embodiments, at least one of R^{4a} and R^{4a} is C₁-C₆ alkyl. In some embodiments, R^{4a} is C₁-C₆ alkyl.

In some embodiments, at least one of R^{4a} and R^{4a′} is CH₃. In some embodiments, R^{4a} is CH₃.

In some embodiments, at least one of R^{4a} and R^{4a′} is halo. In some embodiments, R^{4a} is halo.

In some embodiments, at least one of R^{4a} and R^{4a′} is F or Cl. In some embodiments, R^{4a} is F or Cl.

In some embodiments, at least one of R^{4a} and R^{4a′} is C₆-C₁₀ aryl. In some embodiments, R^{4a} is C₆-C₁₀ aryl.

In some embodiments, at least one of R^{4a} and R^{4a′} is

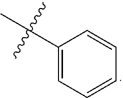 [SMILES: *c1ccccc1]

In some embodiments, R^{4a} is

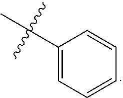 [SMILES: *c1ccccc1]

In some embodiments, at least one of R^{4a} and R^{4a′} is 5- to 10-membered heteroaryl. In some embodiments, R^{4a} is 5- to 10-membered heteroaryl.

In some embodiments, at least one of R^{4a} and R^{4a′} is

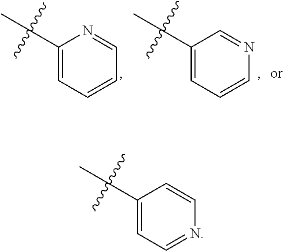 [SMILES: *c1ccccn1 ; *c1cccnc1 ; *c1ccncc1]

In some embodiments, R^{4a} is

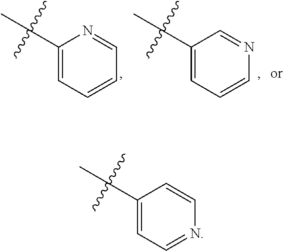 [SMILES: *c1ccccn1 ; *c1cccnc1 ; *c1ccncc1]

In some embodiments, at least one of R^{4a} and R^{4a′} is

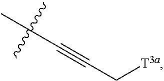

wherein T^{3a} is H, halo, cyano, OR^{7a}, OR^{8a}, C(O)R^{8a}, NR^{7a}R^{8a}, C(O)NR^{7a}R^{8a}, NR^{7a}C(O)R^{8a}, C₆-C₁₀ aryl, 5- to 10-membered heteroaryl, C₃-C₁₂ cycloalkyl, or 4- to 12-membered heterocycloalkyl containing 1-4 heteroatoms selected from N, O, and S, and wherein the C₆-C₁₀ aryl, 5- to 10-membered heteroaryl, C₃-C₁₂ cycloalkyl or 4- to 12-membered heterocycloalkyl is optionally substituted with one or more of halo, hydroxyl, cyano, C₁-C₆ haloalkyl, —SO₂R^{5a}, C₁-C₆ alkoxyl or C₁-C₆ alkyl optionally substituted with one or more of NR^{5a}R^{6a}.

In some embodiments, R^{4a′} is

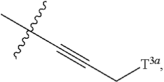

wherein T^{3a} is H, halo, cyano, OR^{7a}, OR^{8a}, C(O)R^{8a}, NR^{7a}R^{8a}, C(O)NR^{7a}R^{8a}, NR^{7a}C(O)R^{8a}, C₆-C₁₀ aryl, 5- to 10-membered heteroaryl, C₃-C₁₂ cycloalkyl, or 4- to 12-membered heterocycloalkyl containing 1-4 heteroatoms selected from N, O, and S, and wherein the C₆-C₁₀ aryl, 5- to 10-membered heteroaryl, C₃-C₁₂ cycloalkyl or 4- to 12-membered heterocycloalkyl is optionally substituted with one or more of halo, hydroxyl, cyano, C₁-C₆ haloalkyl, —SO₂R^{5a}, C₁-C₆ alkoxyl or C₁-C₆ alkyl optionally substituted with one or more of NR^{5a}R^{6a}.

In some embodiments, at least one of R^{4a} and R^{4a′} is

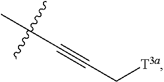

wherein T^{3a} is 5- to 10-membered heteroaryl or 4- to 12-membered heterocycloalkyl optionally substituted with one or more of halo, hydroxyl, C₁-C₆ alkoxyl or C₁-C₆ alkyl.

In some embodiments, R^{4a′} is

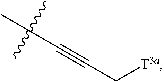

wherein T^{3a} is 5- to 10-membered heteroaryl or 4- to 12-membered heterocycloalkyl optionally substituted with one or more of halo, hydroxyl, C₁-C₆ alkoxyl or C₁-C₆ alkyl.

In some embodiments, at least one of R^{4a} and R^{4a′} is

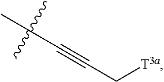

wherein T^{3a} is 5- to 10-membered heteroaryl or 4- to 12-membered heterocycloalkyl optionally substituted with one or more of halo, hydroxyl, C₁-C₆ alkoxyl or C₁-C₆ alkyl and the other of R^{4a} and R^{4a′} is halo, C₁-C₆ alkyl, or OR^{7a}. In some embodiments, R^{7a} is H or C₁-C₆ alkyl optionally substituted with one or more of hydroxyl, amino or mono- or di-alkylamino.

In some embodiments, at least one of R^{4a} and R^{4a′} is —OCH₃, —OCH₂CH₃, or —OCH(CH₃)₂. In some embodiments, at least one of R^{4a} and R^{4a′} is

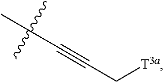

wherein T^{3a} is 5- to 10-membered heteroaryl or 4- to 12-membered heterocycloalkyl optionally substituted with one or more of halo, hydroxyl, C₁-C₆ alkoxyl or C₁-C₆ alkyl and the other of R^{4a} and R^{4a′} is OCH₃, —OCH₂CH₃, or —OCH(CH₃)₂.

In some embodiments, at least one of R^{4a} and R^{4a′}is —OCH₃.

In some embodiments, at least one of R^{4a} and R^{4a′} is

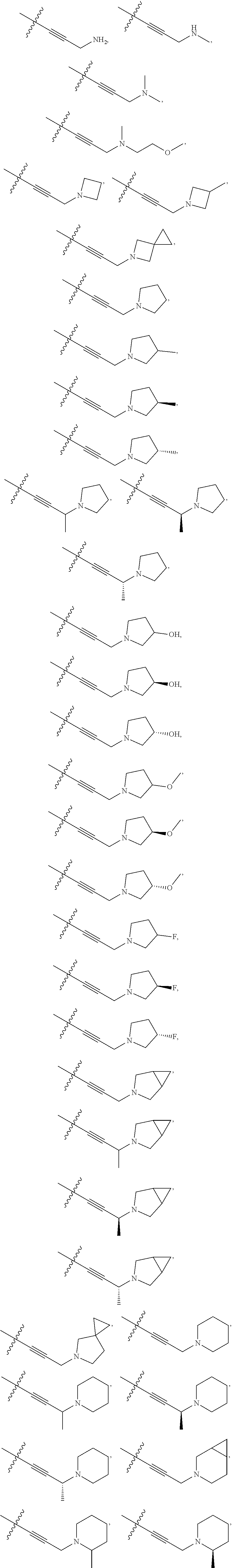

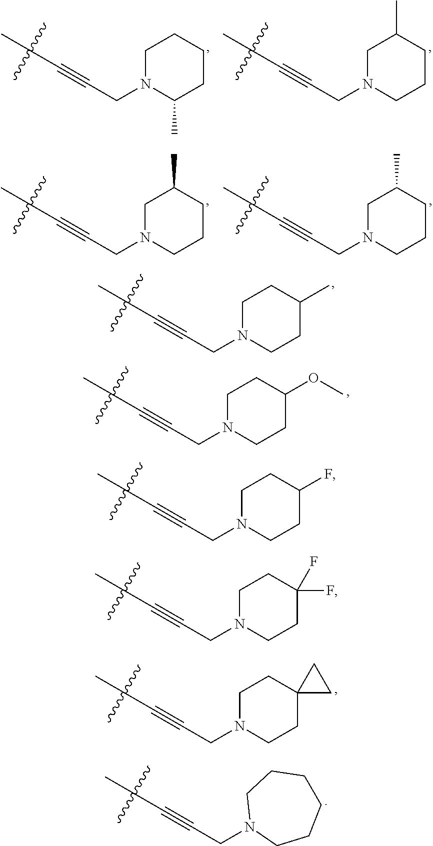

In some embodiments, R^{4a′} is

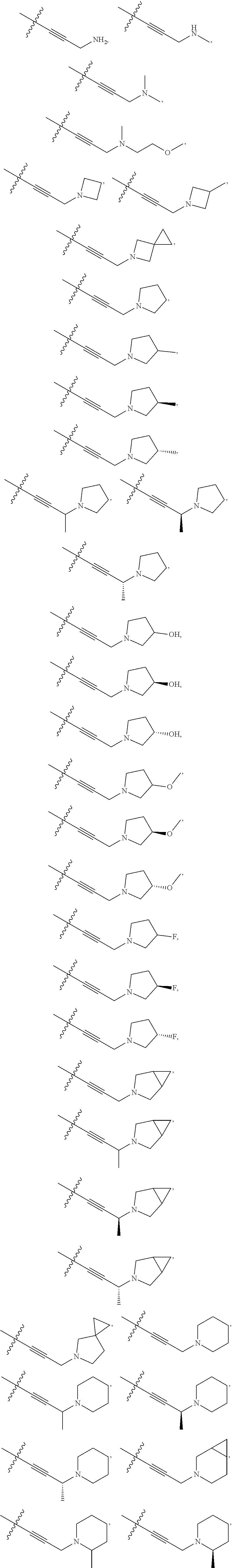

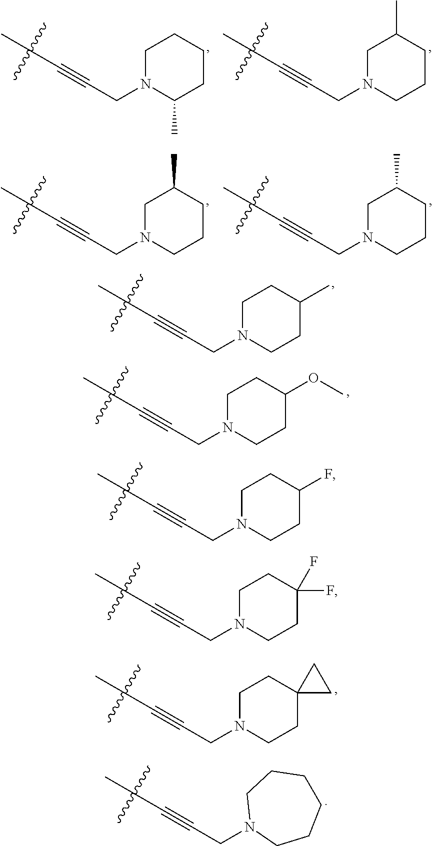

In some embodiments, at least one of R^{4a} and R^{4a′} is OR^{7a}. In some embodiments, R^{4a} is OR^{7a}. In some embodiments, R^{4a′} is OR^{7a}

In some embodiments, at least one of R^{4a} and R^{4a′} is OR^{8a}. In some embodiments, R^{4a′} is OR^{8a}.

In some embodiments, at least one of R^{4a} and R^{4a′} is —CH₂-T^{3a}, wherein T^{3a} is H, halo, cyano, OR^{7a}, OR^{8a}, C(O)R^{8a}, NR^{7a}R^{8a}, C(O)NR^{7a}R^{8a}, NR^{7a}C(O)R^{8a}, C₆-C₁₀ aryl, 5- to 10-membered heteroaryl, C₃-C₁₂ cycloalkyl, or 4- to 12-membered heterocycloalkyl containing 1-4 heteroatoms selected from N, O, and S, and wherein the C₆-C₁₀ aryl, 5- to 10-membered heteroaryl, C₃-C₁₂ cycloalkyl or 4- to 12-membered heterocycloalkyl is optionally substituted with one or more of halo, hydroxyl, cyano, C₁-C₆ haloalkyl, —SO₂R^{5a}, C₁-C₆ alkoxyl or C₁-C₆ alkyl optionally substituted with one or more of NR^{5a}R^{6a}.

In some embodiments, R^{4a′} is —CH₂-T^{3a}, wherein T^{3a} is H, halo, cyano, OR^{7a}, OR^{8a}, C(O)R^{8a}, NR^{7a}R^{8a}, C(O)NR^{7a}R^{8a}, NR^{7a}C(O)R^{8a}, C₆-C₁₀ aryl, 5- to 10-membered heteroaryl, C₃-C₁₂ cycloalkyl, or 4- to 12-membered heterocycloalkyl containing 1-4 heteroatoms selected from N, O, and S, and wherein the C₆-C₁₀ aryl, 5- to 10-membered heteroaryl, C₃-C₁₂ cycloalkyl or 4- to 12-membered heterocycloalkyl is optionally substituted with one or more of halo, hydroxyl, cyano, C₁-C₆ haloalkyl, —SO₂R^{5a}, C₁-C₆ alkoxyl or C₁-C₆ alkyl optionally substituted with one or more of NR^{5a}R^{6a}.

In some embodiments, at least one of R^{4a} and R^{4a′} is —CH₂—OR₈. In some embodiments, R^{4a′} is —CH₂—OR₈.

In some embodiments, at least one of R^{4a} and R^{4a′} is —CH₂—NR₇R₈. In some embodiments, R^{4a′} is —CH₂—NR₇R₈.

In some embodiments, at least one of R^{4a} and R^{4a′} is halo, C₁-C₆ alkyl, or OR^{7a}. In some embodiments, R^{4a} is halo, C₁-C₆ alkyl, or OR^{7a}.

In some embodiments, at least one of R^{4a} and R^{4a′} is C₁-C₆ alkoxyl. In some embodiments, R^{4a} is C₁-C₆ alkoxyl.

In some embodiments, at least one of R^{4a} and R^{4a′} is —OCH₃, —OCH₂CH₃, or —OCH(CH₃)₂. In some embodiments, R^{4a} is —OCH₃, —OCH₂CH₃, or —OCH(CH₃)₂.

In some embodiments, at least one of R^{4a} and R^{4a′} is —OCH₃. In some embodiments, R^{4a} is —OCH₃.

In some embodiments, R^{7a} is H or C₁-C₆ alkyl optionally substituted with one or more of hydroxyl, amino or mono- or di-alkylamino.

In some embodiments, R^{8a} is -Q^{4a}-T^{4a}, in which Q^{4a} is a C₁-C₆ alkylene, C₂-C₆ alkenylene, or C₂-C₆ alkynylene linker optionally substituted with one or more of halo, cyano, hydroxyl, or C₁-C₆ alkoxyl, and T^{4a} is C₃-C₁₂ cycloalkyl, C₆-C₁₀ aryl, or 4- to 12-membered heterocycloalkyl (e.g., 4- to 7-membered heterocycloalkyl) containing 1-4 heteroatoms selected from N, O and S which is optionally substituted with one or more -Q^{5a}-T^{5a}.

In some embodiments, each 4- to 12-membered heterocycloalkyl described herein include, e.g., a 4 to 7-membered monocyclic heterocycloalkyl or 7 to 12-membered bicyclic heterocycloalkyl such as azetidinyl, oxetanyl, thietanyl, pyrrolidinyl, imidazolidinyl, pyrazolidinyl, oxazolidinyl, isoxazolidinyl, triazolidinyl, tetrahyrofuranyl, piperidinyl, 1,2,3,6-tetrahydropyridinyl, piperazinyl, tetrahydro-2H-pyranyl, 3,6-dihydro-2H-pyranyl, tetrahydro-2H-thiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, morpholinyl, 3-azabicyclo[3.1.0]hexan-3-yl, 3-azabicyclo[3.1.0]hexanyl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, and the like.

In some embodiments, R^{8a} is -Q^{4a}-R^{S3a}, in which Q^{4a} is a bond or a C₁-C₆ alkylene linker (e.g., C₂-C₆ alkylene linker) optionally substituted with a hydroxyl and R^{S5a} is 4- to 12-membered heterocycloalkyl (e.g., a 4 to 7-membered monocyclic heterocycloalkyl or 7 to 12-membered bicyclic heterocycloalkyl such as azetidinyl, oxetanyl, thietanyl, pyrrolidinyl, imidazolidinyl, pyrazolidinyl, oxazolidinyl, isoxazolidinyl, triazolidinyl, tetrahyrofuranyl, piperidinyl, 1,2,3,6-tetrahydropyridinyl, piperazinyl, tetrahydro-2H-pyranyl, 3,6-dihydro-2H-pyranyl, tetrahydro-2H-thiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, morpholinyl, 3-azabicyclo[3.1.0]hexan-3-yl, 3-azabicyclo[3.1.0]hexanyl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, and the like), which is optionally substituted with one or more -Q^{5a}-T^{5a}.

In some embodiments, Q^{4a} is C₁-C₆ alkylene linker optionally substituted with a hydroxyl and R^{S3a} is C₃-C₆ cycloalkyl optionally substituted with one or more -Q^{5a}-T^{5a}.

In some embodiments, Q^{4a} is an optionally substituted C₂-C₆ alkenylene or C₂-C₆ alkynylene linker and R^{S3a} is 4- to 12-membered heterocycloalkyl (e.g., a 4 to 7-membered monocyclic heterocycloalkyl or 7 to 12-membered bicyclic heterocycloalkyl such as azetidinyl, oxetanyl, thietanyl, pyrrolidinyl, imidazolidinyl, pyrazolidinyl, oxazolidinyl, isoxazolidinyl, triazolidinyl, tetrahyrofuranyl, piperidinyl, 1,2,3,6-tetrahydropyridinyl, piperazinyl, tetrahydro-2H-pyranyl, 3,6-dihydro-2H-pyranyl, tetrahydro-2H-thiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, morpholinyl, 3-azabicyclo[3.1.0]hexan-3-yl, 3-azabicyclo[3.1.0]hexanyl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, and the like), which is optionally substituted with one or more -Q^{5a}-T^{5a}.

In some embodiments, Q^{4a} is an optionally substituted C₂-C₆ alkenylene or C₂-C₆ alkynylene linker and R^{S3a} is C₃-C₆ cycloalkyl optionally substituted with one or more -Q^{5a}-T^{5a}.

In some embodiments, each Q^{5a} independently is a bond or C₁-C₃ alkylene linker each optionally substituted with one or more of halo, cyano, hydroxyl, or C₁-C₆ alkoxy, and each T^{5a} independently is selected from the group consisting of H, halo, cyano, C₁-C₆ alkyl, C₃-C₁₂cycloalkyl (e.g., C₃-C₈ cycloalkyl), or 4- to 7-membered heterocycloalkyl containing 1-4 heteroatoms selected from N, O, and S.

In some embodiments, each Q independently is a C₂-C₃ alkenylene, or C₂-C₃ alkynylene linker each optionally substituted with one or more of halo, cyano, hydroxyl, or C₁-C₆ alkoxy, and each T^{5a} independently is selected from the group consisting of H, halo, cyano, C₁-C₆ alkyl, C₃-C₁₂cycloalkyl (e.g., C₃-C₈ cycloalkyl), or 4- to 7-membered heterocycloalkyl containing 1-4 heteroatoms selected from N, O, and S.

In some embodiments, -Q^{5a}-T^{5a} is oxo.

In some embodiments, at least one of R^{4a} and R^{4a′} is

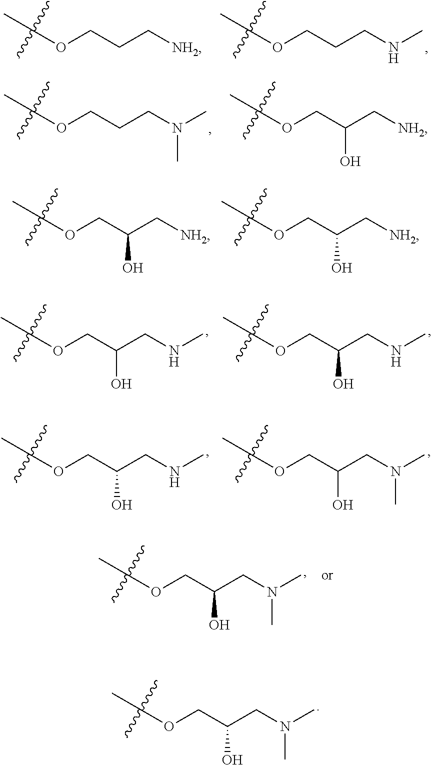

In some embodiments, R^{4a′} is

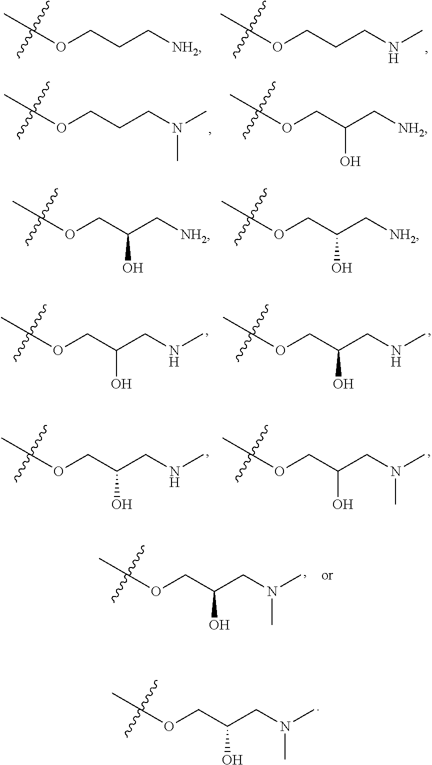

In some embodiments, at least one of R^{4a} and R^{4a′} is

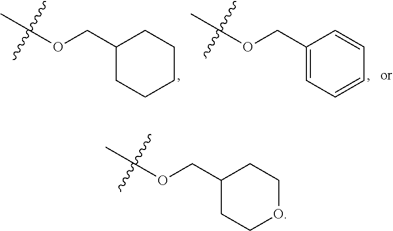 [SMILES: *OCC1CCCCC1 ; *OCc1ccccc1 ; *OCC1CCOCC1]

In some embodiments, R^{4a′} is

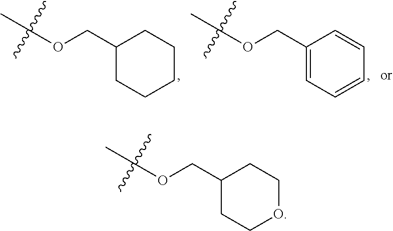 [SMILES: *OCC1CCCCC1 ; *OCc1ccccc1 ; *OCC1CCOCC1]

In some embodiments, at least one of R^{4a} and R^{4a′} is

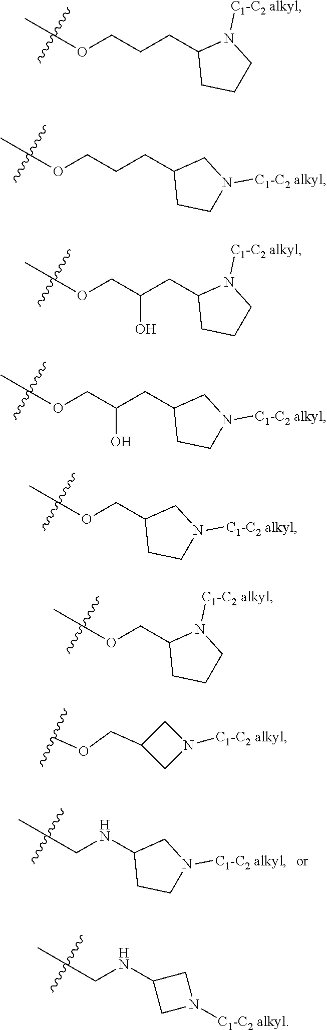

In some embodiments, R^{4a′} is

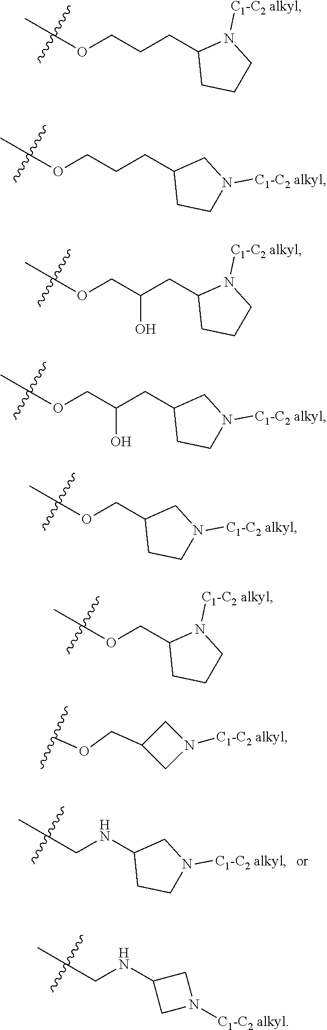

In some embodiments, at least one of R^{4a} and R^{4a′} is

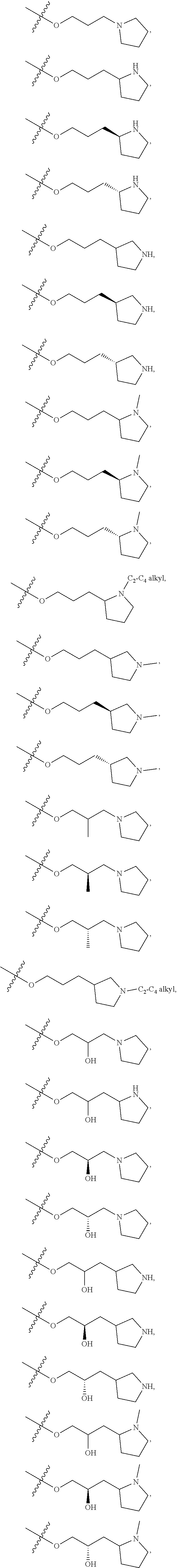

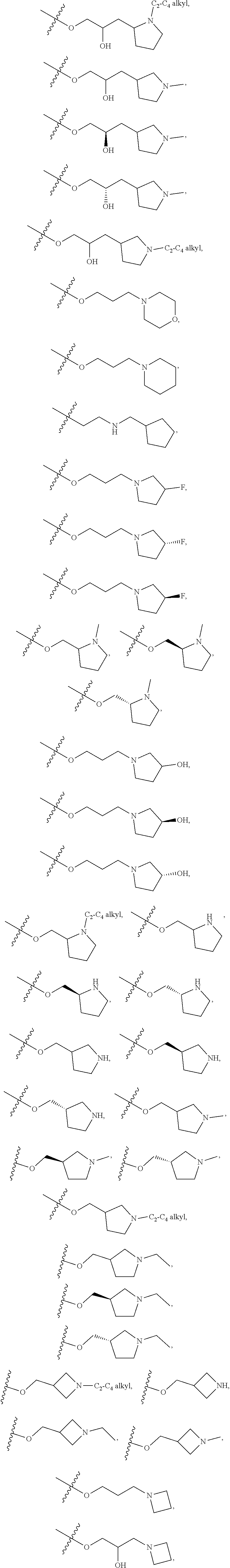

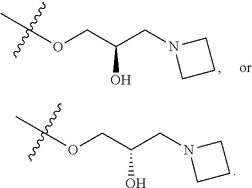 [SMILES: *OC[C@H](O)CN1CCC1 ; *OC[C@@H](O)CN1CCC1]

In some embodiments, R^{4a′} is

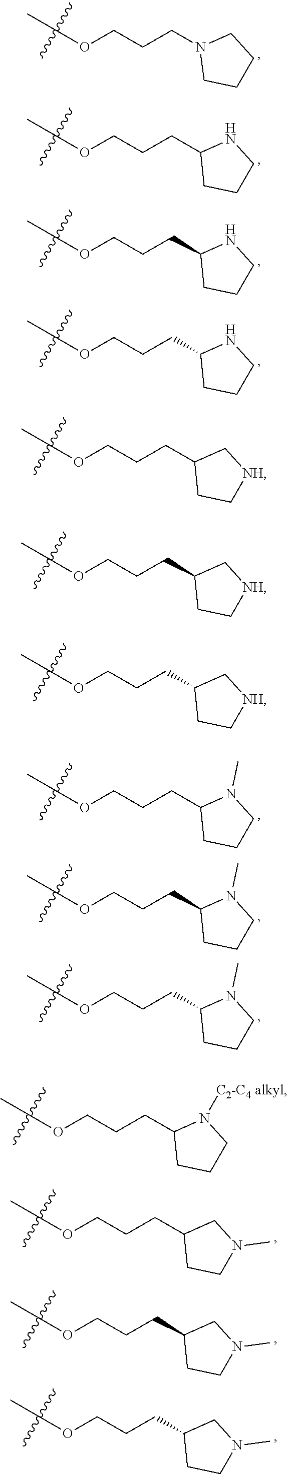

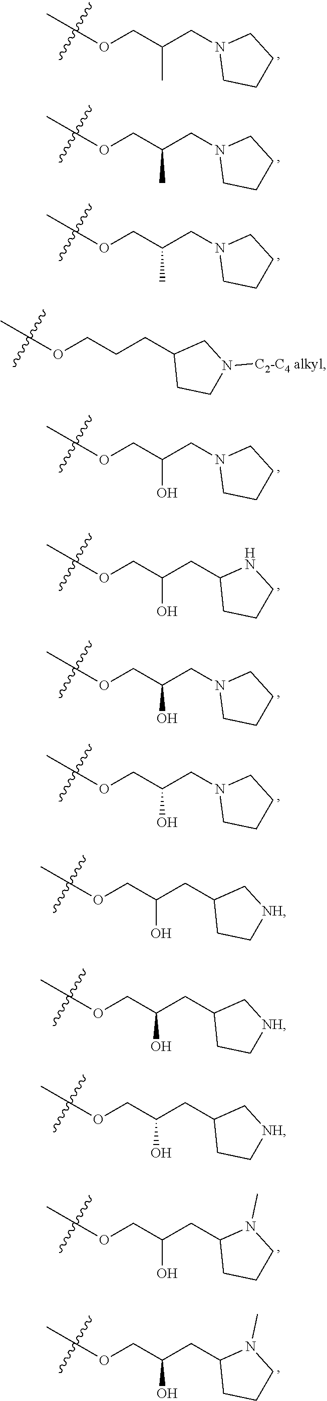

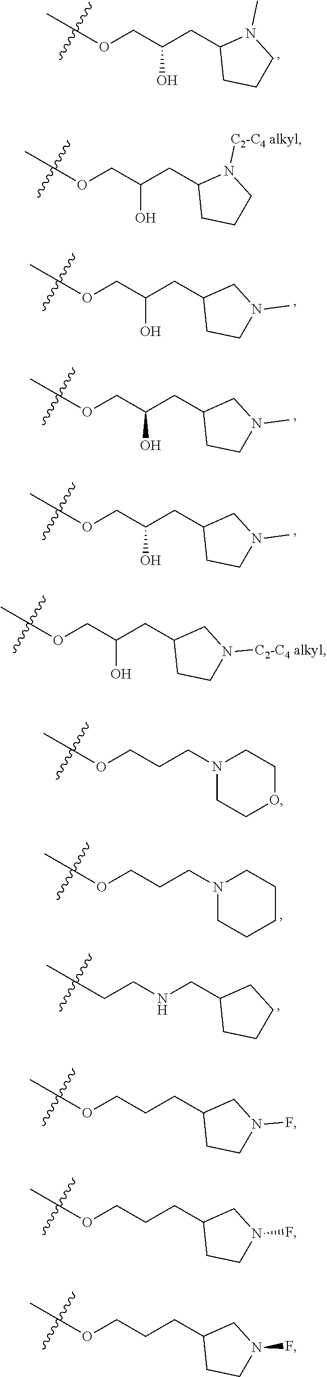

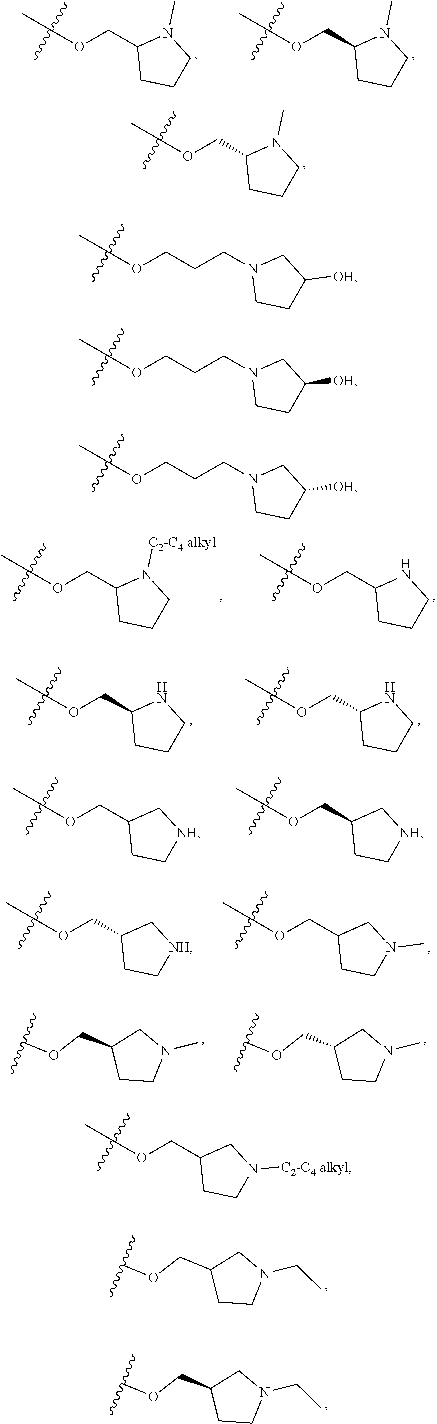

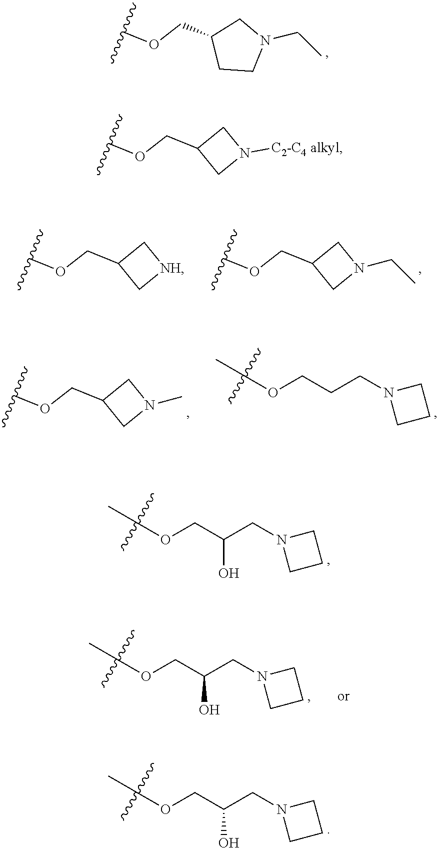

In some embodiments, wherein at least one of R^{4a} and R^{4′} is

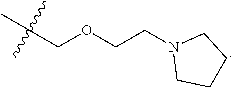 [SMILES: *COCCN1CCCC1]

In some embodiments, R^{4a′} is

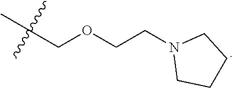 [SMILES: *COCCN1CCCC1]

In some embodiments, wherein at least one of R^{4a} and R^{4a′} is

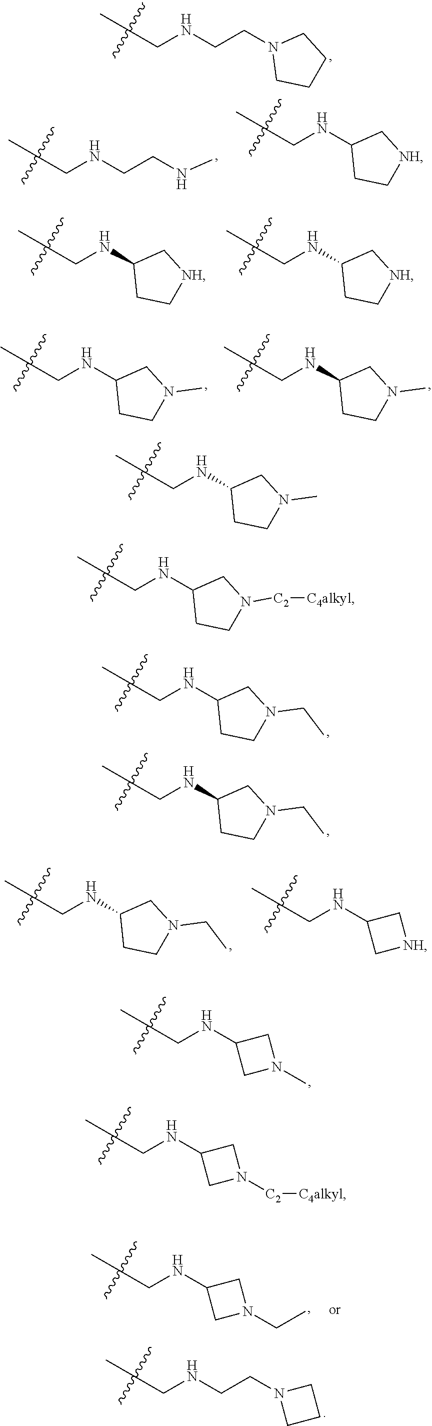

In some embodiments, R^{4a′} is

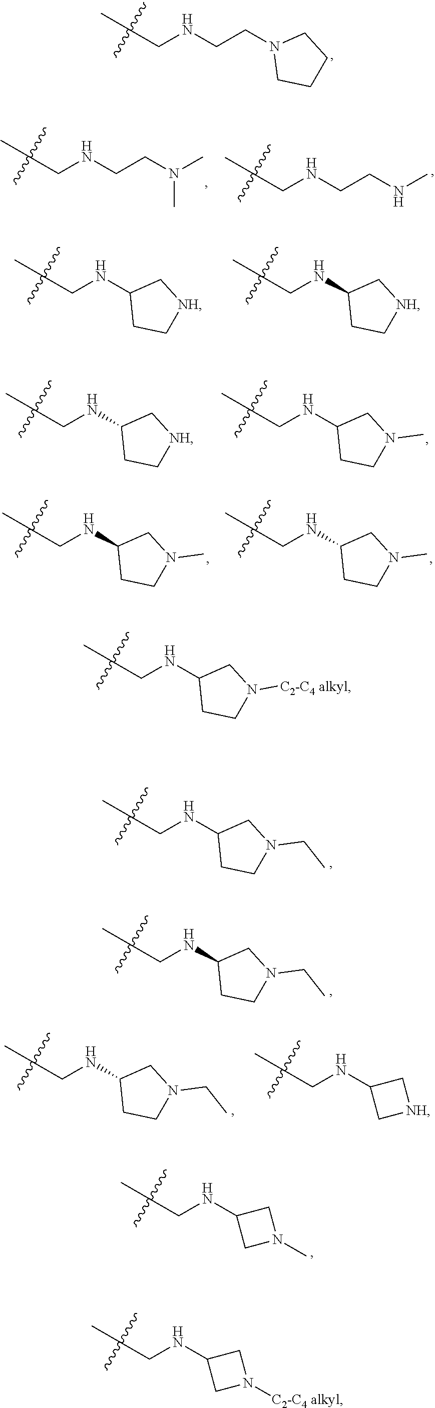

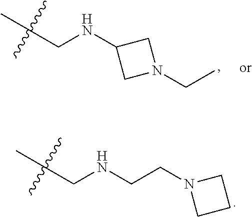

In some embodiments, one of R^{4a} and R^{4a′} is halo, C₁-C₆ alkyl, or OR^{7a}, and the other is

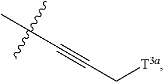

wherein T^{3a} is 5- to 10-membered heteroaryl or 4- to 12-membered heterocycloalkyl optionally substituted with one or more of halo, hydroxyl, C₁-C₆ alkoxyl or C₁-C₆ alkyl.

In some embodiments, R^{4a} is halo, C₁-C₆ alkyl, or OR^{7a}, and R^{4′} is

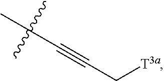

wherein T^{3a} is 5- to 10-membered heteroaryl or 4- to 12-membered heterocycloalkyl optionally substituted with one or more of halo, hydroxyl, C₁-C₆ alkoxyl or C₁-C₆ alkyl.

In some embodiments, one of R^{4a} and R^{4a′} is C₁-C₆ alkoxyl and the other is

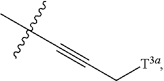

wherein T^{3a} is 5- to 10-membered heteroaryl or 4- to 12-membered heterocycloalkyl optionally substituted with one or more of halo, hydroxyl, C₁-C₆ alkoxyl or C₁-C₆ alkyl.

In some embodiments, R^{4a} is C₁-C₆ alkoxyl, and R^{4a′} is

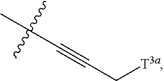

wherein T^{3a} is 5- to 10-membered heteroaryl or 4- to 12-membered heterocycloalkyl optionally substituted with one or more of halo, hydroxyl, C₁-C₆ alkoxyl or C₁-C₆ alkyl.

In some embodiments, one of R^{4a} and R^{4a′} is —OCH₃, and the other is

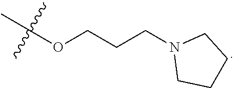 [SMILES: *OCCCN1CCCC1]

In some embodiments, R^{4a} is —OCH₃, and R^{4a′} is

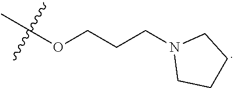 [SMILES: *OCCCN1CCCC1]

In some embodiments, and one of R^{4a} and R^{4a′} is —OCH₃, and the other is

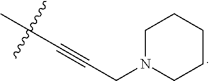 [SMILES: *C#CCN1CCCCC1]

In some embodiments, R^{4a} is —OCH₃, and R^{4a′} is

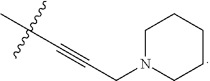 [SMILES: *C#CCN1CCCCC1]

In some embodiments, the compound is of Formula (VIIa′), (VIIb′), (VIIc′), (VIId′), (VIIe′), or (VIIf′):

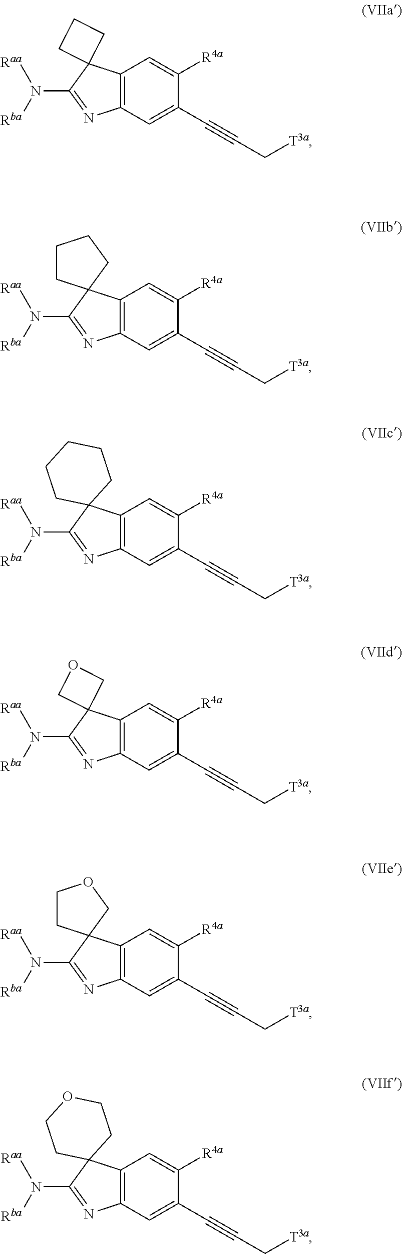

a tautomer thereof, or a pharmaceutically acceptable salt of the compound or the tautomer, wherein
each of R^{aa} and R^{ba} independently is H or R^{S5a}, or R^{aa} and R^{ba} together with the nitrogen atom to which they are attached form a 4- to 12-membered heterocycloalkyl containing 1-4 heteroatoms selected from N, O, and S; in which R^{S5a} is C₁-C₆ alkyl, phenyl, 5- or 6-membered heteroaryl, or 4- to 12-membered heterocycloalkyl containing 1-4 heteroatoms selected from N, O, and S, and each of R^{S4a}, R^{S5a}, and the heterocycloalkyl formed by R^{aa} and R^{ba} is independently optionally substituted with one or more of halo, hydroxyl, oxo, CN, amino, mono- or di-alkylamino, C₁-C₆ alkyl, C₁-C₆ alkoxyl, C₃-C₁₂ cycloalkyl, phenyl, 5- or 6-membered heteroaryl, or 4- to 12-membered heterocycloalkyl containing 1-4 heteroatoms selected from N, O, and S, or alternatively; and
R^{4a} is halo, C₁-C₆ alkyl, or OR^{7a};
T^{3a} is H, halo, cyano, OR^{7a}, OR^{8a}, C(O)R^{8a}, N^{7a}R^{8a}, C(O)NR^{7a}R^{8a}, NR^{7a}C(O)R^{8a}, C₆-C₁₀ aryl, 5- to 10-membered heteroaryl, C₃-C₁₂ cycloalkyl, or 4- to 12-membered heterocycloalkyl containing 1-4 heteroatoms selected from N, O, and S, and wherein the C₆-C₁₀ aryl, 5- to 10-membered heteroaryl, C₃-C₁₂ cycloalkyl or 4- to 12-membered heterocycloalkyl is optionally substituted with one or more of halo, hydroxyl, cyano, C₁-C₆ haloalkyl, —SO₂R^{5a}, C₁-C₆ alkoxyl or C₁-C₆ alkyl optionally substituted with one or more of NR^{5a}R^{6a};
each of R^{5a}, R^{6a}, and R^{7a}, independently, is H or C₁-C₆ alkyl optionally substituted with one or more of halo, cyano, hydroxyl, amino, mono- or di-alkylamino, or C₁-C₆ alkoxyl; and
each R^{8a} independently is -Q^{4a}-T^{4a}, in which Q^{4a} is a bond or C₁-C₆ alkylene, C₂-C₆ alkenylene, or C₂-C₆ alkynylene linker optionally substituted with one or more of halo, cyano, hydroxyl, or C₁-C₆ alkoxyl, and T^{4a} is H, halo, or R^{S3a}, in which R^{S3a} is C₃-C₁₂ cycloalkyl, C₆-C₁₀ aryl, 4- to 12-membered heterocycloalkyl containing 1-4 heteroatoms selected from N, O and S, or a 5- to 10-membered heteroaryl, and R^{S3a} is optionally substituted with one or more -Q^{5a}-T^{5a}, wherein each Q^{5a} independently is a bond or C₁-C₃ alkylene, C₂-C₃ alkenylene, or C₂-C₃ alkynylene linker each optionally substituted with one or more of halo, cyano, hydroxyl, or C₁-C₆ alkoxy, and each T^{5a} independently is selected from the group consisting of H, halo, cyano, C₁-C₆ alkyl, C₃-C₁₂ cycloalkyl, C₆-C₁₀ aryl, 4- to 7-membered heterocycloalkyl containing 1-4 heteroatoms selected from N, O, and S, 5- to 6-membered heteroaryl, OR^{ca}, C(O)R^{ca}, NR^{ca}R^{da} C(O)NR^{ca}R^{da}, S(O)₂R^{ca}, and NR^{ca}C(O)R^{da}, each of R^{ca} and R^{da} independently being H or C₁-C₆ alkyl optionally substituted with one or more halo; or -Q^{5a}-T^{5a} is oxo.

In some embodiments, R^{4a} is —OCH₃.

In some embodiments, T^{3a} is 5- to 10-membered heteroaryl or 4- to 12-membered heterocycloalkyl optionally substituted with one or more of halo, hydroxyl. C₁-C₆ alkoxyl or C₁-C₆ alkyl.

In some embodiments, the compound is of Formula (VIIIa′), (VIIIb′), (VIIIc′), (VIIId′), (VIIIe′), or (VIIIf′):

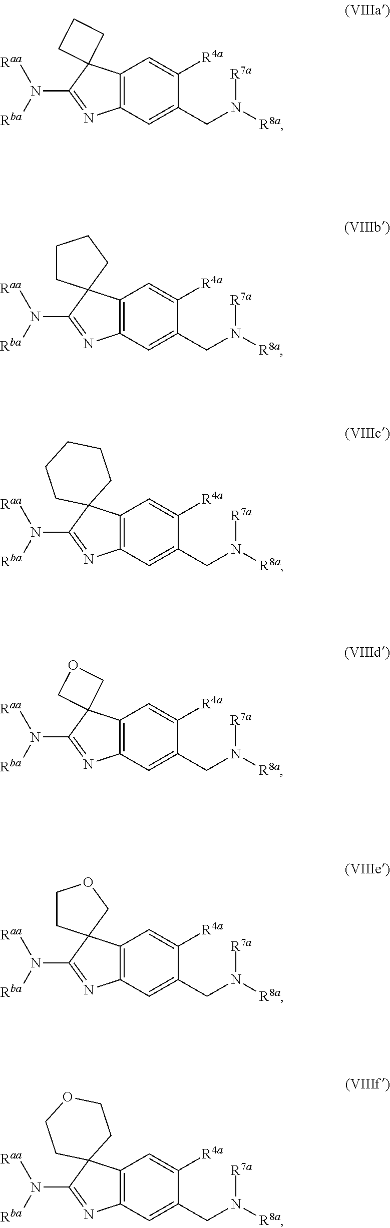

a tautomer thereof, or a pharmaceutically acceptable salt of the compound or the tautomer, wherein
each of R^{aa} and R^{ba} independently is H or R^{S5a}, or R^{aa} and R^{ba} together with the nitrogen atom to which they are attached form a 4- to 12-membered heterocycloalkyl containing 1-4 heteroatoms selected from N, O, and S; in which R^{S5a} is C₁-C₆ alkyl, phenyl, 5- or 6-membered heteroaryl, or 4- to 12-membered heterocycloalkyl containing 1-4 heteroatoms selected from N, O, and S, and each of R^{S4a}, R^{S5a}, and the heterocycloalkyl formed by R^{aa} and R^{ba} is independently optionally substituted with one or more of halo, hydroxyl, oxo, CN, amino, mono- or di-alkylamino, C₁-C₆ alkyl, C₁-C₆ alkoxyl, C₃-C₁₂ cycloalkyl, phenyl, 5- or 6-membered heteroaryl, or 4- to 12-membered heterocycloalkyl containing 1-4 heteroatoms selected from N, O, and S, or alternatively; and
R^{4a} is -Q^{3a}-T^{3a}, in which Q^{3a} is a bond or C₁-C₆ alkylene, C₂-C₆ alkenylene, or C₂-C₆ alkynylene linker optionally substituted with one or more of halo, cyano, hydroxyl, amino, mono- or di-alkylamino, or C₁-C₆ alkoxyl, and T^{3a} is H, halo, cyano, OR^{7a}, OR^{8a}, C(O)R^{8a}, NR^{7a}R^{8a}, C(O)NR^{7a}R^{8a}, NR^{7a}C(O)R^{8a}, C₆-C₁₀ aryl, 5- to 10-membered heteroaryl, C₃-C₁₂ cycloalkyl, or 4- to 12-membered heterocycloalkyl containing 1-4 heteroatoms selected from N, O, and S, and wherein the C₆-C₁₀ aryl, 5- to 10-membered heteroaryl, C₃-C₁₂ cycloalkyl or 4- to 12-membered heterocycloalkyl is optionally substituted with one or more of halo, hydroxyl, cyano, C₁-C₆ haloalkyl, —SO₂R^{5a}, C₁-C₆ alkoxyl or C₁-C₆ alkyl optionally substituted with one or more of NR^{5a}R^{6a};
each of R^{5a}, R^{6a}, and R^{7a}, independently, is H or C₁-C₆ alkyl optionally substituted with one or more of halo, cyano, hydroxyl, amino, mono- or di-alkylamino, or C₁-C₆ alkoxyl; and
each R^{8a} independently is -Q^{4a}-T^{4a}, in which Q^{4a} is a bond or C₁-C₆ alkylene, C₂-C₆ alkenylene, or C₂-C₆ alkynylene linker optionally substituted with one or more of halo, cyano, hydroxyl, or C₁-C₆ alkoxyl, and T^{4a} is H, halo, or R^{S3a}, in which R^{S3a} is C₃-C₁₂ cycloalkyl, C₆-C₁₀ aryl, 4- to 12-membered heterocycloalkyl containing 1-4 heteroatoms selected from N, O and S, or a 5- to 10-membered heteroaryl, and R^{S3a} is optionally substituted with one or more Q^{5a}-T^{5a}, wherein each Q^{5a} independently is a bond or C₁-C₃ alkylene, C₂-C₃ alkenylene, or C₂-C₃ alkynylene linker each optionally substituted with one or more of halo, cyano, hydroxyl, or C₁-C₆ alkoxy, and each T^{5a} independently is selected from the group consisting of H, halo, cyano, C₁-C₆ alkyl, C₃-C₁₂ cycloalkyl, C₆-C₁₀ aryl, 4- to 7-membered heterocycloalkyl containing 1-4 heteroatoms selected from N, O, and S, 5- to 6-membered heteroaryl, OR^{ca}, C(O)R^{ca}, NR^{ca}R^{da}, C(O)NR^{ca}R^{da}, S(O)₂R^{ca}, and NR^{ca}C(O)R^{da}, each of R^{ca} and R^{da} independently being H or C₁-C₆ alkyl optionally substituted with one or more halo; or -Q^{5a}-T^{5a} is oxo.

In some embodiments, R^{4a} is halo, C₁-C₆ alkyl, or OR^{7a}. In some embodiments, R^{4a} is C₁-C₆ alkoxyl. In some embodiments, R^{4a} is —OCH₃.

In some embodiments, the compound is of Formulae (IXa′), (IXb′), (IXc′), (IXd′), (IXe′), or (IXf′):

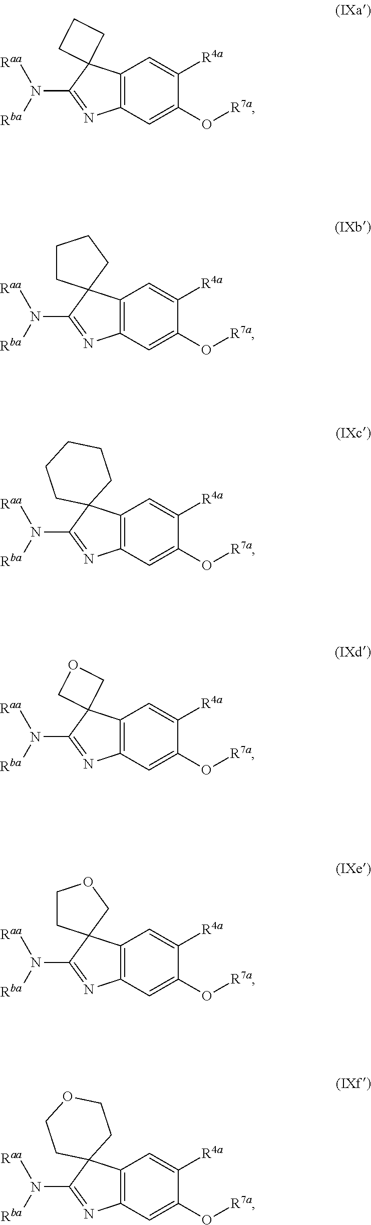

a tautomer thereof, or a pharmaceutically acceptable salt of the compound or the tautomer, wherein
each of R^{aa} and R^{ba} independently is H or R^{S5a}, or R^{aa} and R^{ba} together with the nitrogen atom to which they are attached form a 4- to 12-membered heterocycloalkyl containing 1-4 heteroatoms selected from N, O, and S; in which R^{S5a} is C₁-C₆ alkyl, phenyl, 5- or 6-membered heteroaryl, or 4- to 12-membered heterocycloalkyl containing 1-4 heteroatoms selected from N, O, and S, and each of R^{S4a}, R^{S5a}, and the heterocycloalkyl formed by R^{aa} and R^{ba} is independently optionally substituted with one or more of halo, hydroxyl, oxo, CN, amino, mono- or di-alkylamino, C₁-C₆ alkyl, C₁-C₆ alkoxyl, C₃-C₁₂ cycloalkyl, phenyl, 5- or 6-membered heteroaryl, or 4- to 12-membered heterocycloalkyl containing 1-4 heteroatoms selected from N, O, and S, or alternatively; and
R^{4a} is -Q^{3a}-T^{3a}, in which Q^{3a} is a bond or C₁-C₆ alkylene, C₂-C₆ alkenylene, or C₂-C₆ alkynylene linker optionally substituted with one or more of halo, cyano, hydroxyl, amino, mono- or di-alkylamino, or C₁-C₆ alkoxyl, and T^{3a} is H, halo, cyano, OR^{7a}, OR^{8a}, C(O)R^{8a}, NR^{7a}R^{8a}, C(O)NR^{7a}R^{8a}, NR^{7a}C(O)R^{8a}, C₆-C₁₀ aryl, 5- to 10-membered heteroaryl, C₃-C₁₂ cycloalkyl, or 4- to 12-membered heterocycloalkyl containing 1-4 heteroatoms selected from N, O, and S, and wherein the C₆-C₁₀ aryl, 5- to 10-membered heteroaryl, C₃-C₁₂ cycloalkyl or 4- to 12-membered heterocycloalkyl is optionally substituted with one or more of halo, hydroxyl, cyano, C₁-C₆ haloalkyl, —SO₂R^{5a}, C₁-C₆ alkoxyl or C₁-C₆ alkyl optionally substituted with one or more of NR^{5a}R^{6a};
each of R^{5a}, R^{6a}, and R^{7a}, independently, is H or C₁-C₆ alkyl optionally substituted with one or more of halo, cyano, hydroxyl, amino, mono- or di-alkylamino, or C₁-C₆ alkoxyl; and
each R^{8a} independently is -Q^{4a}-T^{4a}, in which Q^{4a} is a bond or C₁-C₆ alkylene, C₂-C₆ alkenylene, or C₂-C₆ alkynylene linker optionally substituted with one or more of halo, cyano, hydroxyl, or C₁-C₆ alkoxyl, and T^{4a} is H, halo, or R^{S3a}, in which R^{S3a} is C₃-C₁₂ cycloalkyl, C₆-C₁₀ aryl, 4- to 12-membered heterocycloalkyl containing 1-4 heteroatoms selected from N, O and S, or a 5- to 10-membered heteroaryl, and R^{S3a} is optionally substituted with one or more -Q^{5a}-T^{5a}, wherein each Q^{5a} independently is a bond or C₁-C₃ alkylene, C₂-C₃ alkenylene, or C₂-C₃ alkynylene linker each optionally substituted with one or more of halo, cyano, hydroxyl, or C₁-C₆ alkoxy, and each T^{5a} independently is selected from the group consisting of H, halo, cyano, C₁-C₆ alkyl, C₃-C₁₂ cycloalkyl, C₆-C₁₀ aryl, 4- to 7-membered heterocycloalkyl containing 1-4 heteroatoms selected from N, O, and S, 5- to 6-membered heteroaryl, OR^{ca}, C(O)R^{ca}, NR^{ca}R^{da}, C(O)NR^{ca}R^{da}, S(O)₂R^{ca}, and NR^{ca}C(O)R^{da}, each of R^{ca} and R^{da} independently being H or C₁-C₆ alkyl optionally substituted with one or more halo; or -Q^{5a}-T^{5a} is oxo.

In some embodiments, R^{4a} is halo, C₁-C₆ alkyl, or OR^{7a}. In some embodiments, R^{4a} is C₁-C₆ alkoxyl. In some embodiments, R^{4a} is —OCH₃.

In some embodiments, the compound is of Formula (Xa′), (Xb′), (Xc′), (Xd′), (Xe′), or (Xf′):

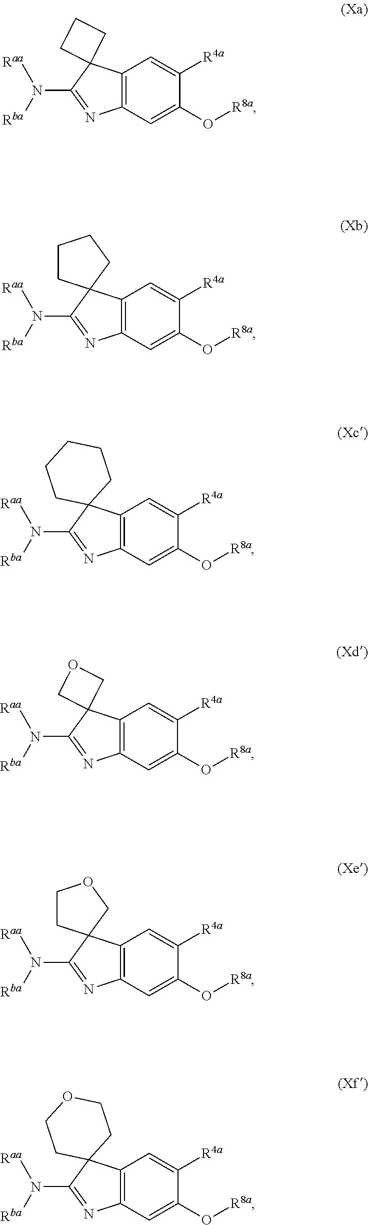

a tautomer thereof, or a pharmaceutically acceptable salt of the compound or the tautomer, wherein
each of R^{aa} and R^{ba} independently is H or R^{S5a}, or R^{aa} and R^{ba} together with the nitrogen atom to which they are attached form a 4- to 12-membered heterocycloalkyl containing 1-4 heteroatoms selected from N, O, and S; in which R^{S5a} is C₁-C₆ alkyl, phenyl, 5- or 6-membered heteroaryl, or 4- to 12-membered heterocycloalkyl containing 1-4 heteroatoms selected from N, O, and S, and each of R^{S4a}, R^{S5a}, and the heterocycloalkyl formed by R^{aa} and R^{ba} is independently optionally substituted with one or more of halo, hydroxyl, oxo, CN, amino, mono- or di-alkylamino, C₁-C₆ alkyl, C₁-C₆ alkoxyl, C₃-C₁₂ cycloalkyl, phenyl, 5- or 6-membered heteroaryl, or 4- to 12-membered heterocycloalkyl containing 1-4 heteroatoms selected from N, O, and S, or alternatively; and
R^{4a} is -Q^{3a}-T^{3a}, in which Q^{3a} is a bond or C₁-C₆ alkylene, C₂-C₆ alkenylene, or C₂-C₆ alkynylene linker optionally substituted with one or more of halo, cyano, hydroxyl, amino, mono- or di-alkylamino, or C₁-C₆ alkoxyl, and T^{3a} is H, halo, cyano, OR^{7a}, OR^{8a}, C(O)R^{8a}, NR^{7a}R^{8a}, C(O)NR^{7a}R^{8a}, NR^{7a}C(O)R^{8a}, C₆-C₁₀ aryl, 5- to 10-membered heteroaryl, C₃-C₁₂ cycloalkyl, or 4- to 12-membered heterocycloalkyl containing 1-4 heteroatoms selected from N, O, and S, and wherein the C₆-C₁₀ aryl, 5- to 10-membered heteroaryl, C₃-C₁₂ cycloalkyl or 4- to 12-membered heterocycloalkyl is optionally substituted with one or more of halo, hydroxyl, cyano, C₁-C₆ haloalkyl, —SO₂R^{5a}, C₁-C₆ alkoxyl or C₁-C₆ alkyl optionally substituted with one or more of NR^{5a}R^{6a};
each of R^{5a}, R^{6a}, and R^{7a}, independently, is H or C₁-C₆ alkyl optionally substituted with one or more of halo, cyano, hydroxyl, amino, mono- or di-alkylamino, or C₁-C₆ alkoxyl; and
each R^{8a} independently is -Q^{4a}-T^{4a}, in which Q^{4a} is a bond or C₁-C₆ alkylene, C₂-C₆ alkenylene, or C₂-C₆ alkynylene linker optionally substituted with one or more of halo, cyano, hydroxyl, or C₁-C₆ alkoxyl, and T^{4a} is H, halo, or R^{S3a}, in which R^{S3a} is C₃-C₁₂ cycloalkyl, C₆-C₁₀ aryl, 4- to 12-membered heterocycloalkyl containing 1-4 heteroatoms selected from N, O and S, or a 5- to 10-membered heteroaryl, and R^{S3a} is optionally substituted with one or more -Q^{5a}-T^{5a}, wherein each Q^{5a} independently is a bond or C₁-C₃ alkylene, C₂-C₃ alkenylene, or C₂-C₃ alkynylene linker each optionally substituted with one or more of halo, cyano, hydroxyl, or C₁-C₆ alkoxy, and each T^{5a} independently is selected from the group consisting of H, halo, cyano, C₁-C₆ alkyl, C₃-C₁₂ cycloalkyl, C₆-C₁₀ aryl, 4- to 7-membered heterocycloalkyl containing 1-4 heteroatoms selected from N, O, and S, 5- to 6-membered heteroaryl, OR^{ca}, C(O)R^{ca}, NR^{ca}R^{da}, C(O)NR^{ca}R^{da}, S(O)₂R^{ca}, and NR^{ca}C(O)R^{da}, each of R_{ca} and R^{da} independently being H or C₁-C₆ alkyl optionally substituted with one or more halo; or -Q^{5a}-T^{5a} is oxo.

In some embodiments, R^{4a} is halo, C₁-C₆ alkyl, or OR^{7a}. In some embodiments, R^{4a} is C₁-C₆ alkoxyl. In some embodiments, R^{4a} is —OCH₃.

In another aspect, the present disclosure provides a method of preventing or treating a blood disorder (e.g., sickle-cell disease) by administering to a subject in need thereof an effective amount of a compound of Formula (I″), (II″), or (III″):

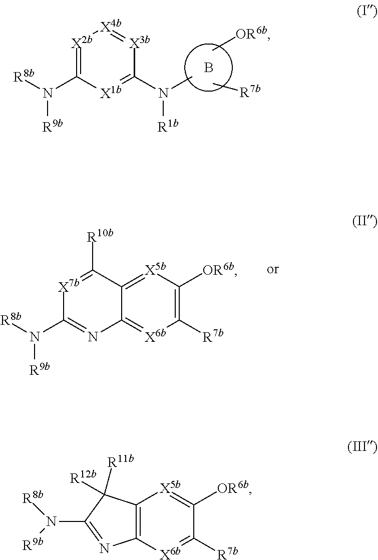

or a tautomer thereof, or a pharmaceutically acceptable salt of the compound or the tautomer, wherein
X^{1b} is N or CR^{2b};
X^{2b} is N or CR^{3b};
X^{3b} is N or CR^{4b};
X^{4b} is N or CR^{5b};
each of X^{5b}, X^{6b} and X^{7b} is independently N or CH;
B is C₆-C₁₀ aryl or 5- to 10-membered heteroaryl;
R^{1b} is H or C₁-C₄ alkyl;
each of R^{2b}, R^{3b}, R^{4b}, and R^{5b}, independently is selected from the group consisting of H, halo, cyano, C₁-C₆ alkoxyl, C₆-C₁₀ aryl, OH, NR^{ab}R^{bb}, C(O)NR^{ab}R^{bb}, NR^{ab}C(O)R^{bb}, C(O)OR^{ab}, OC(O)R^{ab}, OC(O)NR^{ab}R^{bb}, NR^{ab}C(O)OR^{bb}, C₃-C₈ cycloalkyl, 4- to 7-membered heterocycloalkyl, 5- to 6-membered heteroaryl, C₁-C₆ alkyl, C₂-C₆ alkenyl, and C₂-C₆ alkynyl, wherein the C₆-C₁₀ aryl, C₃-C₈ cycloalkyl, 4- to 7-membered heterocycloalkyl, 5- to 6-membered heteroaryl, C₁-C₆ alkoxyl, C₁-C₆ alkyl, C₂-C₆ alkenyl, and C₂-C₆ alkynyl, are each optionally substituted with one or more of halo, OR^{ab}, or NR^{ab}R^{bb}, in which each of R^{ab} and R^{bb} independently is H or C₁-C₆ alkyl;
R^{6b} is Q^{1b}-T^{1b}, in which Q^{1b} is a bond, or C₁-C₆ alkylene, C₂-C₆ alkenylene, or C₂-C₆ alkynylene linker each optionally substituted with one or more of halo, cyano, hydroxyl, oxo, or C₁-C₆ alkoxyl, and T^{1b} is H, halo, cyano, or R^{S1b}, in which R^{S1b} is C₃-C₈ cycloalkyl, phenyl, 4- to 12-membered heterocycloalkyl containing 1-4 heteroatoms selected from N, O, and S, or a 5- or 6-membered heteroaryl and R^{S1b} is optionally substituted with one or more of halo, C₁-C₆ alkyl, C₂-C₆ alkenyl, C₂-C₆ alkynyl, hydroxyl, oxo, —C(O)R^{cb}, —C(O)OR^{cb}, —SO₂R^{cb}, —SO₂N(R^{cb})₂, —NR^{cb}C(O)R^{db}, —C(O)NR^{cb}R^{db}, —NR^{cb}C(O)OR^{db}, —OC(O)NR^{cb}R^{db}, NR^{cb}R^{db}, or C₁-C₆ alkoxyl, in which each of R^{cb} and R^{db} independently is H or C₁-C₆ alkyl;
R^{7b} is -Q^{2b}-T^{2b}, in which Q^{2b} is a bond, C(O)NR^{eb}, or NR^{eb}C(O), R^{eb} being H or C₁-C₆ alkyl and T^{2b} is 5- to 10-membered heteroaryl or 4- to 12-membered heterocycloalkyl, and wherein the 5- to 10-membered heteroaryl or 4- to 12-membered heterocycloalkyl is optionally substituted with one or more -Q^{3b}-T^{3b}, wherein each Q^{3b} independently is a bond or C₁-C₃ alkylene linker each optionally substituted with one or more of halo, cyano, hydroxyl, or C₁-C₆ alkoxy, and each T^{3b} independently is selected from the group consisting of H, halo, cyano, C₁-C₆ alkyl, C₂-C₆ alkenyl, C₂-C₆ alkynyl, C₃-C₈ cycloalkyl, C₆-C₁₀ aryl, 4- to 7-membered heterocycloalkyl containing 1-4 heteroatoms selected from N, O, and S, 5- to 6-membered heteroaryl, OR^{fb}, C(O)R^{fb}, C(O)OR^{fb}, OC(O)R^{fb}, S(O)₂R^{fb}, NR^{fb}R^{gb}, OC(O)NR^{fb}R^{gb}, NR^{fb}C(O)OR^{gb}, C(O)NR^{fb}R^{gb}, and NR^{fb}C(O)R^{gb}, each of R^{fb} and R^{gb} independently being H or C₁-C₆ alkyl, in which the C₃-C₈ cycloalkyl, C₆-C₁₀ aryl, 4- to 7-membered heterocycloalkyl or 5- to 6-membered heteroaryl is optionally substituted with one or more halo, cyano, hydroxyl, C₁-C₆ alkyl, C₂-C₆ alkenyl, C₂-C₆ alkynyl, or C₁-C₆ alkoxy; or -Q^{3b}-T^{3b} is oxo;
R^{8b} is H or C₁-C₆ alkyl;
R^{9b} is -Q^{4b}-T^{4b}, in which Q^{4b} is a bond or C₁-C₆ alkylene, C₂-C₆ alkenylene, or C₂-C₆ alkynylene linker each optionally substituted with one or more of halo, cyano, hydroxyl, or C₁-C₆ alkoxyl, and T^{4b} is H, halo, OR^{hb}, NR^{hb}R^{ib}, NR^{hb}C(O)R^{ib}, C(O)NR^{hb}R^{ib}, C(O)R^{hb}, C(O)OR^{hb}, NR^{hb}C(O)OR^{ib}, OC(O)NR^{hb}R^{ib}, S(O)₂R^{hb}, S(O)₂NR^{hb}R^{ib}, or R^{S2b}, in which each of R^{hb} and R^{ib} independently is H or C₁-C₆ alkyl, and R^{S2b} is C₃-C₈ cycloalkyl, C₆-C₁₀ aryl, 4- to 12-membered heterocycloalkyl containing 1-4 heteroatoms selected from N, O, and S, or a 5- to 10-membered heteroaryl, and R^{S2b} is optionally substituted with one or more -Q^{5b}-T^{5b}, wherein each Q^{5b} independently is a bond or C₁-C₃ alkylene linker each optionally substituted with one or more of halo, cyano, hydroxyl, or C₁-C₆ alkoxy, and each T^{5b} independently is selected from the group consisting of H, halo, cyano, C₁-C₆ alkyl, C₂-C₆ alkenyl, C₂-C₆ alkynyl, C₃-C₈ cycloalkyl, C₆-C₁₀ aryl, 4- to 7-membered heterocycloalkyl containing 1-4 heteroatoms selected from N, O, and S, 5- to 6-membered heteroaryl, OR^{jb}, C(O)R^{jb}, C(O)OR^{jb}, OC(O)R^{jb}, S(O)₂R^{jb}, NR^{jb}R^{kb}, OC(O)NR^{jb}R^{kb}, NR^{jb}C(O)OR^{kb}, C(O)NR^{jb}R^{kb}, and NR^{jb}C(O)R^{kb}, each of R^{jb} and R^{kb} independently being H or C₁-C₆ alkyl; or -Q^{5b}-T^{5b} is oxo;

R^{10b} is 4- to 12-membered heterocycloalkyl containing 1-4 heteroatoms selected from N, O, and S, which is optionally substituted with one or more halo, cyano, hydroxyl, oxo, amino, mono- or di-alkylamino, C₁-C₆ alkyl, C₂-C₆ alkenyl, C₂-C₆ alkynyl, or C₁-C₆ alkoxy; and
R^{11b} and R^{12b} together with the carbon atom to which they are attached form a C₃-C₁₂ cycloalkyl or 4- to 12-membered heterocycloalkyl containing 1-4 heteroatoms selected from N, O, and S, wherein the C₃-C₁₂ cycloalkyl or 4- to 12-membered heterocycloalkyl is optionally substituted with one or more of halo, C₁-C₆ alkyl, C₂-C₆ alkenyl, C₂-C₆ alkynyl, hydroxyl, oxo, amino, mono- or di-alkylamino, or C₁-C₆ alkoxyl.

The compounds of Formulae (I″)-(III″) may have one or more of the following features when applicable.

In some embodiments, the EHMT2 inhibitor is a compound is of Formula (I″).

In some embodiments, at least one of X^{1b}, X^{2b}, X^{3b} and X^{4b} is N.

In some embodiments, X^{1b} and X^{3b} are N.

In some embodiments, X^{1b} and X^{3b} are N, X^{2b} is CR^{3b} and X^{4b} is CR^{5b}.

In some embodiments,

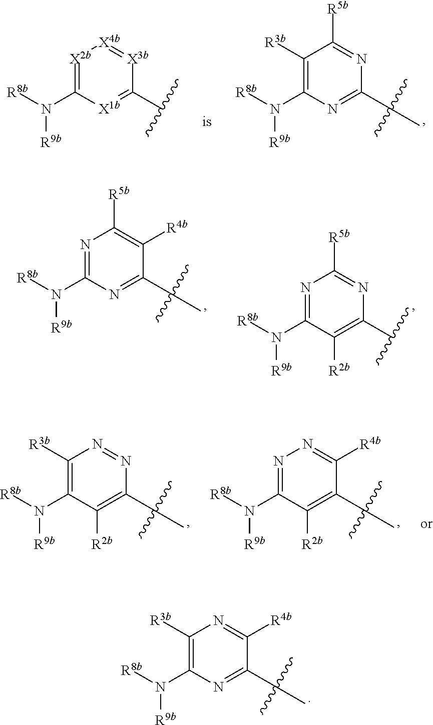

In embodiments,

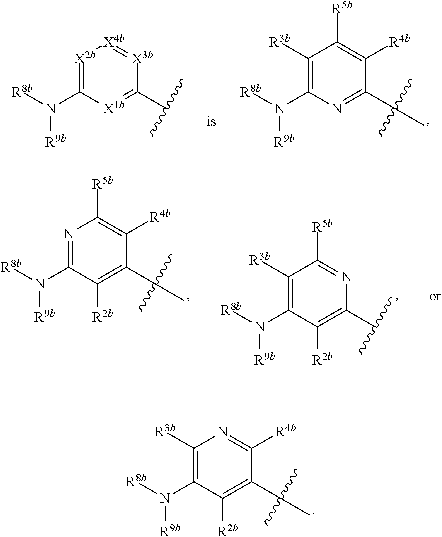

In some embodiments, ring B is phenyl or 6-membered heteroaryl.

In embodiments,

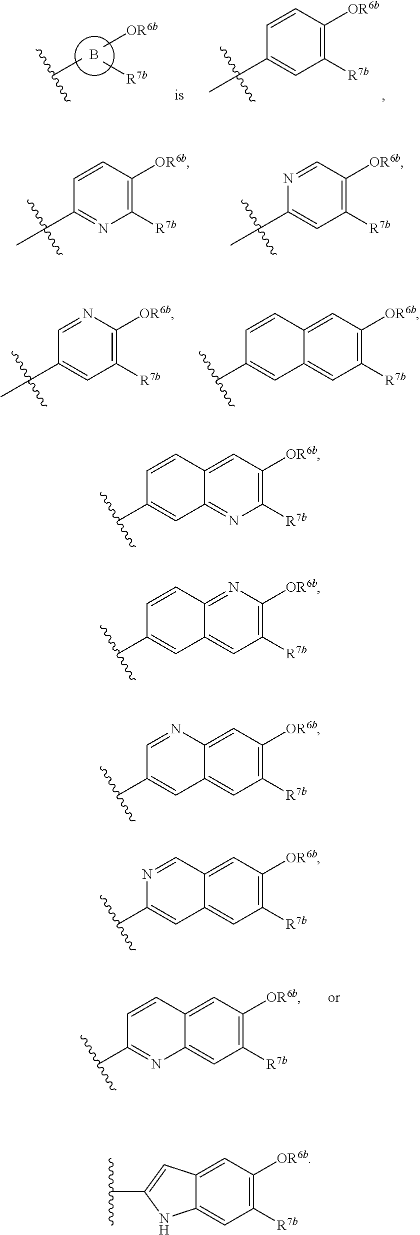

In some embodiments, ring B is phenyl or pyridyl.

In some embodiments, the EHMT2 inhibitor is a compound of Formula (Ia″), (Ib″), (Ic″), or (Id″):

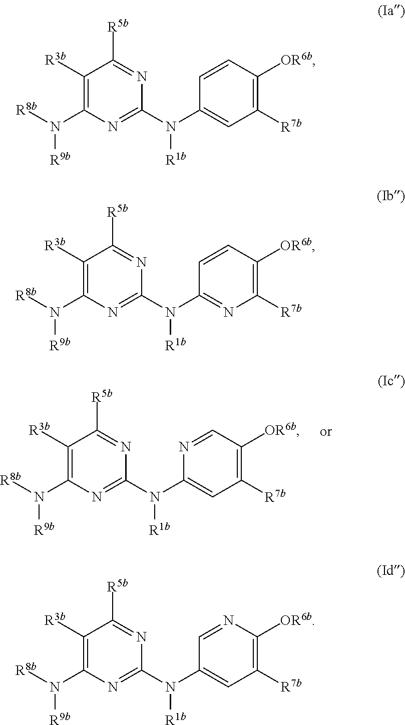

In some embodiments, at most one of R^{3b} and R^{5b} is not H.

In some embodiments, at least one of R^{3b} and R^{5b} is not H.

In some embodiments, R^{3b} is H or halo.

In some embodiments, the EHMT2 inhibitor is a compound of Formula (Ie″), (If″), (Ig″), or (Ih″):

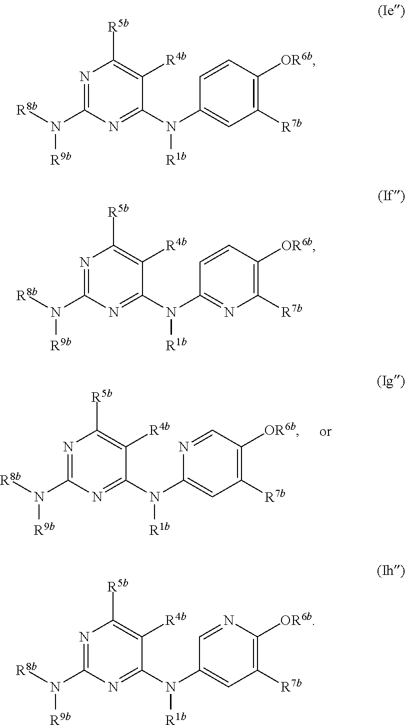

In some embodiments, at most one of R^{4b} and R^{5b} is not H.

In some embodiments, at least one of R^{4b} and R^{5b} is not H.

In some embodiments, R^{4b} is H, C₁-C₆ alkyl, or halo.

In some embodiments, the EHMT2 inhibitor is a compound of Formula (Ii″), (Ij″), (Ik″), or (Il″):

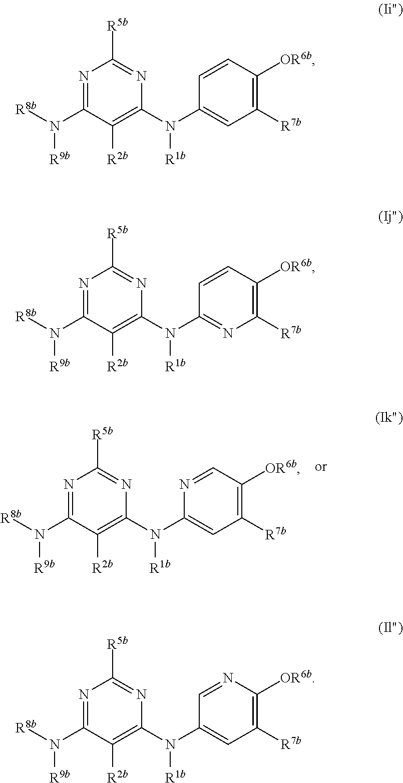

In some embodiments, at most one of R^{2b} and R^{5b} is not H.

In some embodiments, at least one of R^{2b} and R^{5b} is not H.

In some embodiments, R^{2b} is H, C₁-C₆ alkyl, or halo.

In some embodiments, R^{5b} is C₁-C₆ alkyl.

In some embodiments, the EHMT2 inhibitor is a compound is of Formula (II″).

In some embodiments, each of X^{5b}, X^{6b} and X^{7b} is CH.

In some embodiments, at least one of X^{5b}, X^{6b} and X^{7b} is N.

In some embodiments, at most one of X^{5b}, X^{6b} and X^{7b} is N.

In some embodiments, R^{10b} is optionally substituted 4- to 7-membered heterocycloalkyl containing 1-4 heteroatoms selected from N, O, and S.

In some embodiments, R^{10b} is connected to the bicyclic group of Formula (II″) via a carbon-carbon bond.

In some embodiments, R^{10b} is connected to the bicyclic group of Formula (II″) via a carbon-nitrogen bond.

In some embodiments, the compound is of Formula (III″).

In some embodiments, R^{11b} and R^{12b} together with the carbon atom to which they are attached form a 4- to 7-membered heterocycloalkyl containing 1-4 heteroatoms selected from N, O, and S, wherein the 4- to 7-membered heterocycloalkyl is optionally substituted with one or more of halo, C₁-C₆ alkyl, hydroxyl, oxo, amino, mono- or di-alkylamino, or C₁-C₆ alkoxyl.

In some embodiments, R^{11b} and R^{12b} together with the carbon atom to which they are attached form a C₄-C₈ cycloalkyl which is optionally substituted with one or more of halo, C₁-C₆ alkyl, hydroxyl, oxo, amino, mono- or di-alkylamino, or C₁-C₆ alkoxyl.

In some embodiments, each of X^{5b} and X^{6b} is CH.

In some embodiments, each of X^{5b} and X^{6b} is N.

In some embodiments, one of X^{5b} and X^{6b} is CH and the other is CH.

In some embodiments, R^{6b} is -Q^{1b}-T^{1b}, in which Q^{1b} is a bond or C₁-C₆ alkylene linker optionally substituted with one or more of halo, and T^{1b} is H, halo, cyano, or R^{S1b}, in which R^{S1b} is C₃-C₈ cycloalkyl, phenyl, 4- to 12-membered heterocycloalkyl containing 1-4 heteroatoms selected from N, O, and S, or a 5- or 6-membered heteroaryl and R^{S1b} is optionally substituted with one or more of halo, C₁-C₆ alkyl, hydroxyl, oxo, NR^{cb}R^{db}, or C₁-C₆ alkoxyl.

In some embodiments, R^{bb} is C₁-C₆ alkyl optionally substituted with one or more of halo, cyano, hydroxyl, or C₁-C₆ alkoxyl.

In some embodiments, R^{bb} is unsubstituted C₁-C₆ alkyl.

In some embodiments, R^{7b} is -Q^{2b}-T^{2b}, in which Q^{2b} is a bond or C(O)NR^{cb}, and T^{2b} is 5- to 10-membered heteroaryl or 4- to 12-membered heterocycloalkyl, wherein the 5- to 10-membered heteroaryl or 4- to 12-membered heterocycloalkyl is optionally substituted with one or more -Q^{3b}-T^{3b}.

In some embodiments, Q^{2b} is a bond.

In some embodiments, T^{2b} is 4- to 12-membered heterocycloalkyl containing 1-4 heteroatoms selected from N, O, and S, which is optionally substituted with one or more -Q^{3b}-T^{3b}.

In some embodiments, T^{2b} is 8- to 12-membered bicyclic heterocycloalkyl that comprises a 5- or 6-membered aryl or heteroaryl ring fused with a non-aromatic ring.

In some embodiments, T^{2b} is 8- to 12-membered bicyclic heterocycloalkyl that comprises a 5- or 6-membered aryl or heteroaryl ring fused with a non-aromatic ring, in which the 5- or 6-membered aryl or heteroaryl ring is connected to Q^{2b}.

In some embodiments, T^{2b} is 5- to 10-membered heteroaryl.

In some embodiments, T^{2b} is selected from

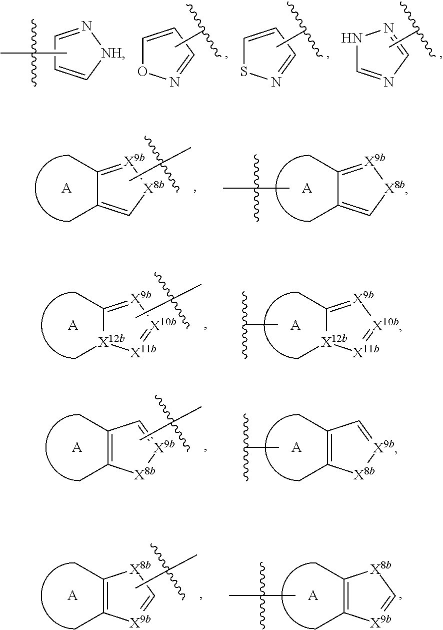

and tautomers thereof, each of which is optionally substituted with one or more -Q^{3b}-T^{3b}, wherein X^{8b} is NH, O, or S, each of X^{9b}, X^{10b}, X^{11b}, and X^{12b} is independently CH or N, and at least one of X^{9b}, X^{10b}, X^{11b}, and X^{12b} is N, and ring A is a C₅-C₈ cycloalkyl, phenyl, 6-membered heteroaryl, or 4- to 8-membered heterocycloalkyl containing 1-4 heteroatoms selected from N, O, and S.

In some embodiments, T^{2b} is selected from

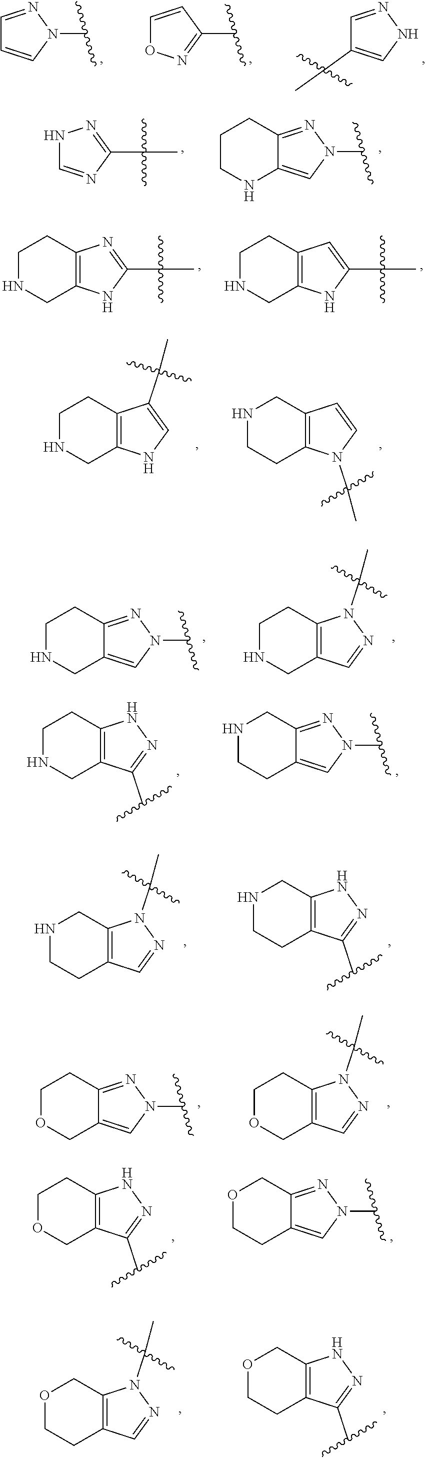

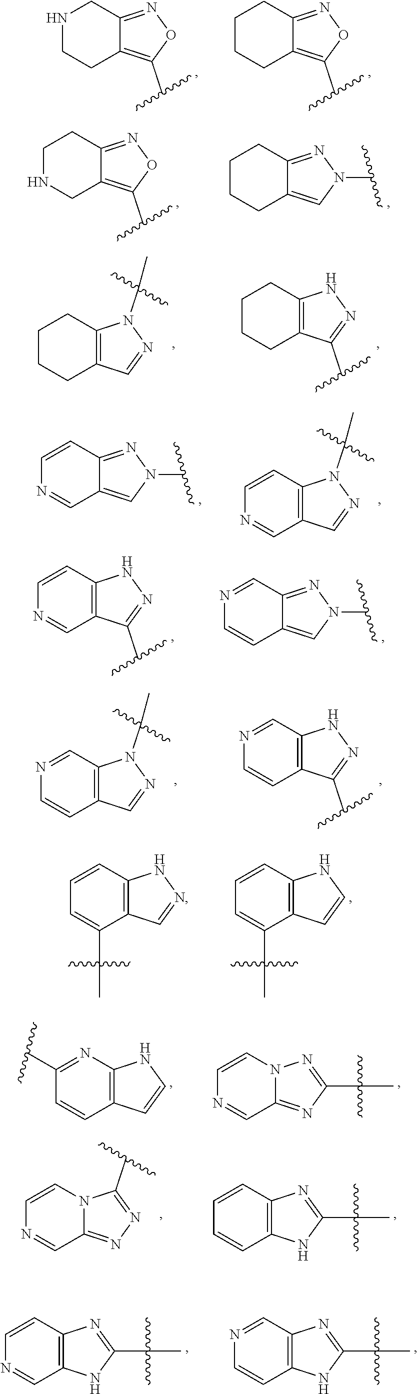

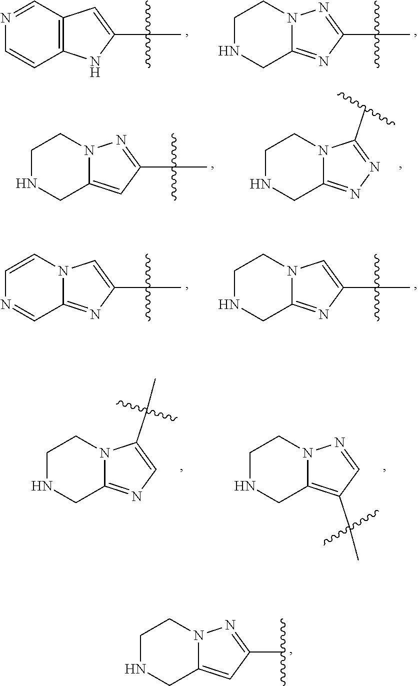

and tautomers thereof, each of which is optionally substituted with one or more -Q^{3b}T^{3b}.

In some embodiments, each Q^{3b} independently is a bond or C₁-C₃ alkylene linker each optionally substituted with one or more of halo, cyano, hydroxyl, or C₁-C₆ alkoxy, and each T^{3b} independently is selected from the group consisting of H, C₁-C₆ alkyl, C₃-C₈ cycloalkyl, 4- to 7-membered heterocycloalkyl, OR^{fb}, C(O)R^{fb}, C(O)OR^{fb}, NR^{fb}R^{gb}, C(O)NR^{fb}R^{gb}, and NR^{fb}C(O)R^{gb}, in which the C₃-C₈ cycloalkyl or 4- to 7-membered heterocycloalkyl is optionally substituted with one or more halo, cyano, hydroxyl, C₁-C₆ alkyl or C₁-C₆ alkoxy.

In some embodiments, at least one of R^{8b} and R^{9b} is H.

In some embodiments, each of R^{8b} and R^{9b} is H.

In some embodiments, R^{8b} is H.

In some embodiments, R^{9b} is -Q^{4b}-T^{4b}, in which Q⁴¹ is a bond or C₁-C₆ alkylene linker optionally substituted with one or more of halo, cyano, hydroxyl, or C₁-C₆ alkoxyl, and T^{4b} is H, halo, OR^{bb}, NR^{bb}R^{ib}, NR^{bb}C(O)R^{ib}, C(O)NR^{bb}R^{ib}, C(O)R^{bb}, C(O)OR^{bb}, or R^{S2b}, in which R^{S2b} is C₃-C₈ cycloalkyl or 4- to 7-membered heterocycloalkyl, and R^{S2b} is optionally substituted with one or more -Q^{5b}-T^{5b}.

In some embodiments, each Q^{5b} independently is a bond or C₁-C₃ alkylene linker.

In some embodiments, each T^{5b} independently is selected from the group consisting of H, halo, cyano, C₁-C₆ alkyl, OR^{jb}, C(O)R^{jb}, C(O)OR^{jb}, NR^{jb}R^{kb}, C(O)NR^{jb}R^{kb}, and NR^{jb}C(O)R^{kb}.

In some embodiments, R^{9b} is C₁-C₃ alkyl.

In some embodiments, for the methods disclosed herein, the EHMT2 inhibitor is of Formula (I′″), (II′″), or (III′″):

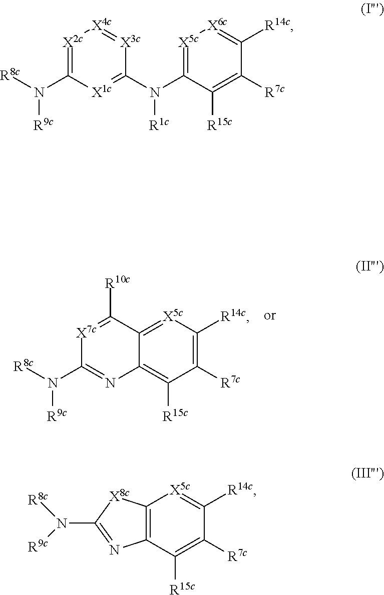

tautomers thereof, and pharmaceutically acceptable salts of the compounds and the tautomers, wherein
X^{1c} is N or CR^{2c};
X^{2c} is N or CR^{3c};
X^{3c} is N or CR^{4c};
X^{4c} is N or CR^{5c};
each of X^{5c}, X^{6c} and X^{7c} is independently N or CH:
X^{8c} is NR^{13c} or CR^{11c}R^{12c};
R^{1c} is H or C₁-C₄ alkyl;
each of R^{2c}, R^{3c}, R^{4c}, and R^{5c}, independently is selected from the group consisting of H, halo, cyano, C₁-C₆ alkoxyl, C₆-C₁₀ aryl, OH, NR^{ac}R^{bc}, C(O)NR^{ac}R^{bc}, NR^{ac}C(O)R^{bc}, C(O)OR^{ac}, OC(O)R^{ac}, OC(O)NR^{ac}R^{bc}, NR^{ac}C(O)OR^{bc}, C₃-C₈ cycloalkyl, 4- to 7-membered heterocycloalkyl, 5- to 6-membered heteroaryl, C₁-C₆ alkyl, C₂-C₆ alkenyl, and C₂-C₆ alkynyl, wherein the C₆-C₁₀ aryl, C₃-C₈ cycloalkyl, 4- to 7-membered heterocycloalkyl, 5- to 6-membered heteroaryl, C₁-C₆ alkoxyl, C₁-C₆ alkyl, C₂-C₆ alkenyl, and C₂-C₆ alkynyl, are each optionally substituted with one or more of halo, OR^{ac}, or NR^{ac}R^{bc}, in which each of R^{ac} and R^{bc} independently is H or C₁-C₆ alkyl;
R^{6c} is -Q^{1c}-T^{1c}, in which Q^{1c} is a bond, or C₁-C₆ alkylene, C₂-C₆ alkenylene, or C₂-C₆ alkynylene linker each optionally substituted with one or more of halo, cyano, hydroxyl, oxo, or C₁-C₆ alkoxyl, and T_{1c} is H, halo, cyano, or R^{S1c}, in which R^{S1c} is C₃-C₈ cycloalkyl, phenyl, 4- to 12-membered heterocycloalkyl containing 1-4 heteroatoms selected from N, O, and S, or a 5- or 6-membered heteroaryl and R^{S1c} is optionally substituted with one or more of halo, C₁-C₆ alkyl, C₂-C₆ alkenyl, C₂-C₆ alkynyl, hydroxyl, oxo, —C(O)R^{cc}, —C(O)OR^{cc}, —SO₂R^{cc}, —SO₂N(R^{cc})₂, —NR^{cc}C(O)R^{dc}, —C(O)NR^{cc}R^{dc}, —NR^{cc}C(O)OR^{dc}, —OC(O)NR^{cc}R^{dc}, NR^{cc}R^{dc}, or C₁-C₆ alkoxyl, in which each of R^{cc} and R^{dc} independently is H or C₁-C₆ alkyl;
R^{7c} is -Q^{2c}-T^{2c}, in which Q^{2c} is a bond, C₁-C₆ alkylene, C₂-C₆ alkenylene, or C₂-C₆ alkynylene linker optionally substituted with one or more of halo, cyano, hydroxyl, amino, mono- or di-alkylamino, and T^{2c} is H, halo, cyano, OR^{cc}, OR^{fc}, C(O)R^{fc}, NR^{cc}R^{fc}, C(O)NR^{cc}R^{fc}, NR^{cc}C(O)R^{fc}, C₆-C₁₀ aryl, 5- to 10-membered heteroaryl, C₃-C₁₂ cycloalkyl, or 4- to 12-membered heterocycloalkyl, and wherein the C₆-C₁₀ aryl, 5- to 10-membered heteroaryl, C₃-C₁₂ cycloalkyl, or 4- to 12-membered heterocycloalkyl is optionally substituted with one or more -Q^{3c}-T^{3c}, wherein each Q^{3c} independently is a bond or C₁-C₃ alkylene linker each optionally substituted with one or more of halo, cyano, hydroxyl, or C₁-C₆ alkoxy, and each T^{3c} independently is selected from the group consisting of H, halo, cyano, C₁-C₆ alkyl, C₂-C₆ alkenyl, C₂-C₆ alkynyl, C₃-C₈ cycloalkyl, C₆-C₁₀ aryl, 4- to 7-membered heterocycloalkyl containing 1-4 heteroatoms selected from N, O, and S, 5- to 6-membered heteroaryl, OR^{cc}, OR^{fc}, C(O)R^{fc}, C(O)OR^{fc}, OC(O)R^{f}c, S(O)₂R^{fc}, NR^{fc}R^{gc}, OC(O)NR^{fc}R^{gc}, NR^{fc}C(O)OR^{gc}, C(O)NR^{fc}R^{gc}, and NR^{fc}C(O)R^{gc}; or -Q^{3c}-T^{3c} is oxo;
each R^{cc} independently is H or C₁-C₆ alkyl optionally substituted with one or more of halo, cyano, hydroxyl, amino, mono- or di-alkylamino, or C₁-C₆ alkoxyl;
each of R^{fc} and R^{gc}, independently, is -Q^{6c}-T⁶, in which Q^{6c} is a bond or C₁-C₆ alkylene, C₂-C₆ alkenylene, or C₂-C₆ alkynylene linker each optionally substituted with one or more of halo, cyano, hydroxyl, or C₁-C₆ alkoxyl, and T⁶ is H, halo, OR^{m1c}, NR^{m1c}R^{m2c}, NR^{m1c}C(O)R^{m2c}, C(O)NR^{m1c}R^{m2c}, C(O)R^{m1c}, C(O)OR^{m1c}, NR^{m1c}C(O)OR^{m2c}, OC(O)NR^{m1c}R^{m2c}, S(O)₂R^{m1c}, S(O)₂NR^{m1c}R^{m2c}, or R^{S1c}, in which each of R^{m1c} and R^{m2c} independently is H, C₁-C₆ alkyl, or (C₁-C₆ alkyl)-R^{S3c}, and R^{S3c} is C₃-C₈ cycloalkyl, C₆-C₁₀ aryl, 4- to 12-membered heterocycloalkyl containing 1-4 heteroatoms selected from N, O, and S, or a 5- to 10-membered heteroaryl, and R^{S3c} is optionally substituted with one or more -Q^{7c}-T^{7c}, wherein each Q^{7c} independently is a bond or C₁-C₃ alkylene linker each optionally substituted with one or more of halo, cyano, hydroxyl, or C₁-C₆ alkoxy, and each T^{7c} independently is selected from the group consisting of H, halo, cyano, C₁-C₆ alkyl, C₂-C₆ alkenyl, C₂-C₆ alkynyl, C₃-C₈ cycloalkyl, C₆-C₁₀ aryl, 4- to 7-membered heterocycloalkyl containing 1-4 heteroatoms selected from N, O, and S, 5- to 6-membered heteroaryl, OR^{n1c}, C(O)R^{n1c}, C(O)OR^{n1c}, OC(O)R^{n1c}, S(O)₂R^{n1c}, NR^{n1c}CR^{n2c}, OC(O)NR^{n1c}R^{n2c}, NR^{n1c}C(O)OR^{n2c}, C(O)NR^{n1c}R^{n2c}, and NR^{n1c}C(O)R^{n2c}, each of R^{n1c} and R^{n2c} independently being H or C₁-C₆ alkyl; or -Q^{7c}-T^{7c} is oxo;
R^{8c} is H or C₁-C₆ alkyl;
R^{8c} is -Q^{4c}-T^{4c}, in which Q^{4c} is a bond or C₁-C₆ alkylene, C₂-C₆ alkenylene, or C₂-C₆ alkynylene linker each optionally substituted with one or more of halo, cyano, hydroxyl, or C₁-C₆ alkoxyl, and T^{4c} is H, halo, OR^{hc}, NR^{hc}R^{ic}, NR^{hc}C(O)R^{ic}, C(O)NR^{hc}R^{ic}, C(O)R^{hc}, C(O)OR^{hc}, NR^{hc}C(O)OR^{ic}, OC(O)NR^{hc}R^{ic}, S(O)₂R^{hc}, S(O)₂NR^{hc}R^{ic}, or R^{S2c}, in which each of R^{hc} and R^{ic} independently is H or C₁-C₆ alkyl, and R^{S2c} is C₃-C₈ cycloalkyl, C₆-C₁₀ aryl, 4- to 12-membered heterocycloalkyl containing 1-4 heteroatoms selected from N, O, and S, or a 5- to 10-membered heteroaryl, and R^{S2c} is optionally substituted with one or more -Q^{5c}-T^{5c}, wherein each Q^{5c} independently is a bond or C₁-C₃ alkylene linker each optionally substituted with one or more of halo, cyano, hydroxyl, or C₁-C₆ alkoxy, and each T^{5c} independently is selected from the group consisting of H, halo, cyano, C₁-C₆ alkyl, C₂-C₆ alkenyl, C₂-C₆ alkynyl, C₃-C₈ cycloalkyl, C₆-C₁₀ aryl, 4- to 7-membered heterocycloalkyl containing 1-4 heteroatoms selected from N, O, and S, 5- to 6-membered heteroaryl, OR^{jc}, C(O)R^{jc}, C(O)OR^{jc}, OC(O)R^{jc}, S(O)₂R^{jc}, NR^{jc}R^{kc}, OC(O)NR^{jc}R^{kc}, NR^{jc}C(O)OR^{kc}, C(O)NR^{jc}R^{kc}, and NR^{jc}C(O)R^{kc}, each of R^{jc} and R^{kc} independently being H or C₁-C₆ alkyl; or -Q^{5c}T^{5c} is oxo;
R^{10c} is halo, C₁-C₆ alkyl, C₂-C₆ alkenyl, C₂-C₆ alkynyl, C₃-C₈ cycloalkyl, or 4- to 12-membered heterocycloalkyl containing 1-4 heteroatoms selected from N, O, and S, wherein each of the C₁-C₆ alkyl, C₂-C₆ alkenyl, C₂-C₆ alkynyl, C₃-C₈ cycloalkyl, and 4- to 12-membered heterocycloalkyl is optionally substituted with one or more halo, cyano, hydroxyl, oxo, amino, mono- or di-alkylamino, C₁-C₆ alkyl, C₂-C₆ alkenyl, C₂-C₆ alkynyl, C₁-C₆ alkoxy, C(O)NR^{jc}R^{kc}, or NR^{jc}C(O)R^{kc};
R^{11c} and R^{12c} together with the carbon atom to which they are attached form a C₃-C₁₂ cycloalkyl or 4- to 12-membered heterocycloalkyl containing 1-4 heteroatoms selected from N, O, and S, wherein the C₃-C₁₂ cycloalkyl or 4- to 12-membered heterocycloalkyl is optionally substituted with one or more of halo, C₁-C₆ alkyl, C₂-C₆ alkenyl, C₂-C₆ alkynyl, hydroxyl, oxo, amino, mono- or di-alkylamino, or C₁-C₆ alkoxyl;
R^{13c} is H, C₁-C₆ alkyl, C₂-C₆ alkenyl, C₂-C₆ alkynyl, C₃-C₁₂ cycloalkyl, or 4- to 12-membered heterocycloalkyl containing 1-4 heteroatoms selected from N, O, and S; and
each of R^{14c} and R^{15c}, independently, is H, halo, cyano, C₁-C₆ alkyl optionally substituted with one or more of halo or cyano, C₂-C₆ alkenyl optionally substituted with one or more of halo or cyano, C₂-C₆ alkynyl optionally substituted with one or more of halo or cyano, C₃-C₈ cycloalkyl optionally substituted with one or more of halo or cyano, or —OR^{6c}.

In some embodiments, for the methods disclosed herein, the EHMT2 inhibitor is of Formula (I′″), (II′″), or (III′″), a tautomer thereof, or a pharmaceutically acceptable salt of the compound or the tautomer, wherein
X^{1c} is N or CR^{2c};
X^{2c} is N or CR^{3c};
X^{3c} is N or CR^{4c};
X^{4c} is N or CR^{5c};
each of X^{5c}, X^{6c} and X^{7c} is independently N or CH;
X^{8c} is NR^{13c} or CR^{11c}R^{12c};
R^{1c} is H or C₁-C₄ alkyl;
each of R^{2c}, R^{3c}, R^{4c}, and R^{5c}, independently is selected from the group consisting of H, halo, cyano, C₁-C₆ alkoxyl, C₆-C₁₀ aryl, OH, NR^{ac}R^{bc}, C(O)NR^{ac}R^{bc}, NR^{ac}C(O)R^{bc}, C(O)OR^{ac}, OC(O)R^{ac}, OC(O)NR^{ac}R^{bc}, NR^{ac}C(O)OR^{bc}, C₃-C₈ cycloalkyl, 4- to 7-membered heterocycloalkyl, 5- to 6-membered heteroaryl, C₁-C₆ alkyl, C₂-C₆ alkenyl, and C₂-C₆ alkynyl, wherein the C₆-C₁₀ aryl, C₃-C₈ cycloalkyl, 4- to 7-membered heterocycloalkyl, 5- to 6-membered heteroaryl, C₁-C₆ alkoxyl, C₁-C₆ alkyl, C₂-C₆ alkenyl, and C₂-C₆ alkynyl, are each optionally substituted with one or more of halo, OR^{ac}, or NR^{ac}R^{bc}, in which each of R^{ac} and R^{bc} independently is H or C₁-C₆ alkyl;
R^{6c} is -Q^{1c}-T^{1c}, in which Q^{1c} is a bond, or C₁-C₆ alkylene, C₂-C₆ alkenylene, or C₂-C₆ alkynylene linker each optionally substituted with one or more of halo, cyano, hydroxyl, oxo, or C₁-C₆ alkoxyl, and T^{1c} is H, halo, cyano, or R^{S1c}, in which R^{S1c} is C₃-C₈ cycloalkyl, phenyl, 4- to 12-membered heterocycloalkyl containing 1-4 heteroatoms selected from N, O, and S, or a 5- or 6-membered heteroaryl and R^{S1c} is optionally substituted with one or more of halo, C₁-C₆ alkyl, C₂-C₆ alkenyl, C₂-C₆ alkynyl, hydroxyl, oxo, —C(O)R^{cc}, —C(O)OR^{cc}, —SO₂R^{cc}, —SO₂N(R^{cc})₂, —NR^{cc}C(O)R^{dc}, —C(O)NR^{cc}R^{dc}, —NR^{cc}C(O)OR^{dc}, —OC(O)NR^{cc}R^{dc}, NR^{cc}R^{dc}, or C₁-C₆ alkoxyl, in which each of R^{cc} and R^{dc} independently is H or C₁-C₆ alkyl;
R^{7c} is -Q^{2c}-T^{2c}, in which Q^{2c} is a bond, C₁-C₆ alkylene, C₂-C₆ alkenylene, or C₂-C₆ alkynylene linker optionally substituted with one or more of halo, cyano, hydroxyl, amino, mono- or di-alkylamino, and T^{2c} is H, halo, cyano, OR^{cc}, OR^{fc}, C(O)R^{fc}, NR^{cc}R^{fc}, C(O)NR^{cc}R^{fc}, NR^{cc}C(O)R^{fc}, C₆-C₁₀ aryl, 5- to 10-membered heteroaryl, C₃-C₁₂ cycloalkyl, or 4- to 12-membered heterocycloalkyl, and wherein the C₆-C₁₀ aryl, 5- to 10-membered heteroaryl, C₃-C₁₂ cycloalkyl, or 4- to 12-membered heterocycloalkyl is optionally substituted with one or more -Q^{3c}-T^{3c}, wherein each Q³ independently is a bond or C₁-C₃ alkylene linker each optionally substituted with one or more of halo, cyano, hydroxyl, or C₁-C₆ alkoxy, and each T^{3c} independently is selected from the group consisting of H, halo, cyano, C₁-C₆ alkyl, C₂-C₆ alkenyl, C₂-C₆ alkynyl, C₃-C₈ cycloalkyl, C₆-C₁₀ aryl, 4- to 7-membered heterocycloalkyl containing 1-4 heteroatoms selected from N, O, and S, 5- to 6-membered heteroaryl, OR^{cc}, OR^{fc}, C(O)R^{fc}, C(O)OR^{fc}, OC(O)R^{fc}, S(O)₂R^{fc}, NR^{fc}R^{gc}, OC(O)NR^{fc}R^{gc}, NR^{fc}C(O)OR^{gc}, C(O)NR^{fc}R^{gc}, and NR^{fc}C(O)R^{gc}; or -Q^{3c}-T^{3c} is oxo;
each R^{cc} independently is H or C₁-C₆ alkyl optionally substituted with one or more of halo, cyano, hydroxyl, amino, mono- or di-alkylamino, or C₁-C₆ alkoxyl;
each of R^{fc} and R^{gc}, independently, is -Q^{6c}-T^{6c}, in which Q^{6c} is a bond or C₁-C₆ alkylene, C₂-C₆ alkenylene, or C₂-C₆ alkynylene linker each optionally substituted with one or more of halo, cyano, hydroxyl, or C₁-C₆ alkoxyl, and T^{6c} is H, halo, OR^{m1c}, NR^{m1c}R^{m2c}, NR^{m1c}C(O)R^{m2c}, C(O)NR^{m1c}R^{m2c}, C(O)R^{m1c}, C(O)OR^{m1c}, NR^{m1c}C(O)OR^{m2c}, OC(O)NR^{m1c}R^{m2c}, S(O)₂R^{m1c}, S(O)₂NR^{m1c}R^{m2c}, or R^{S3c}, in which each of R^{m1c} and R^{m2c} independently is H or C₁-C₆ alkyl, and R^{S3c} is C₃-C₈ cycloalkyl, C₆-C₁₀ aryl, 4- to 12-membered heterocycloalkyl containing 1-4 heteroatoms selected from N, O, and S, or a 5- to 10-membered heteroaryl, and R^{S3c} is optionally substituted with one or more -Q^{7c}-T^{7c}, wherein each Q^{7c} independently is a bond or C₁-C₃ alkylene linker each optionally substituted with one or more of halo, cyano, hydroxyl, or C₁-C₆ alkoxy, and each T^{7c} independently is selected from the group consisting of H, halo, cyano, C₁-C₆ alkyl, C₂-C₆ alkenyl, C₂-C₆ alkynyl, C₃-C₈ cycloalkyl, C₆-C₁₀ aryl, 4- to 7-membered heterocycloalkyl containing 1-4 heteroatoms selected from N, O, and S, 5- to 6-membered heteroaryl, OR^{n1c}, C(O)R^{n1c}, C(O)OR^{n1c}, OC(O)R^{n1c}, S(O)₂R^{n1c}, NR^{n1c}R^{n2c}, OC(O)NR^{n1c}R^{n2c}, NR^{n1c}C(O)OR^{n2c}, C(O)NR^{n1c}R^{n2c}, and NR^{n1c}C(O)R^{n2c}, each of R^{n1c} and R^{n2c} independently being H or C₁-C₆ alkyl; or Q^{7c}-T^{7c} is oxo;
R^{8c} is H or C₁-C₆ alkyl;
R^{9c} is -Q^{4c}-T^{4c}, in which Q^{4c} is a bond or C₁-C₆ alkylene, C₂-C₆ alkenylene, or C₂-C₆ alkynylene linker each optionally substituted with one or more of halo, cyano, hydroxyl, or C₁-C₆ alkoxyl, and T^{4c} is H, halo, OR^{hc}, NR^{hc}R^{ic}, NR^{hc}C(O)R^{ic}, C(O)NR^{hc}R^{ic}, C(O)R^{hc}, C(O)OR^{hc}, NR^{hc}C(O)OR^{ic}, OC(O)NR^{hc}R^{ic}, S(O)₂R^{hc}, S(O)₂NR^{hc}R^{ic}, or R^{S2c}, in which each of R^{hc} and R^{ic} independently is H or C₁-C₆ alkyl, and R^{S2c} is C₃-C₈ cycloalkyl, C₆-C₁₀ aryl, 4- to 12-membered heterocycloalkyl containing 1-4 heteroatoms selected from N, O, and S, or a 5- to 10-membered heteroaryl, and R^{S2c} is optionally substituted with one or more -Q^{5c}-T^{5c}, wherein each Q^{5c} independently is a bond or C₁-C₃ alkylene linker each optionally substituted with one or more of halo, cyano, hydroxyl, or C₁-C₆ alkoxy, and each T⁵ independently is selected from the group consisting of H, halo, cyano, C₁-C₆ alkyl, C₂-C₆ alkenyl, C₂-C₆ alkynyl, C₃-C₈ cycloalkyl, C₆-C₁₀ aryl, 4- to 7-membered heterocycloalkyl containing 1-4 heteroatoms selected from N, O, and S, 5- to 6-membered heteroaryl, OR^{jc}, C(O)R^{jc}, C(O)OR^{jc}, OC(O)R^{jc}, S(O)₂R^{jc}, NR^{jc}R^{kc}, OC(O)NR^{jc}R^{kc}, NR^{jc}C(O)OR^{kc}, C(O)NR^{jc}R^{kc}, and NR^{jc}C(O)R^{kc}, each of R^{jc} and R^{kc} independently being H or C₁-C₆ alkyl; or -Q^{5c}-T^{5c} is oxo;
R^{10C} is halo, C₁-C₆ alkyl, C₂-C₆ alkenyl, C₂-C₆ alkynyl, C₃-C₈ cycloalkyl, or 4- to 12-membered heterocycloalkyl containing 1-4 heteroatoms selected from N, O, and S, wherein each of the C₁-C₆ alkyl, C₂-C₆ alkenyl, C₂-C₆ alkynyl, C₃-C₈ cycloalkyl, and 4- to 12-membered heterocycloalkyl is optionally substituted with one or more halo, cyano, hydroxyl, oxo, amino, mono- or di-alkylamino, C₁-C₆ alkyl, C₂-C₆ alkenyl, C₂-C₆ alkynyl, C₁-C₆ alkoxy, C(O)NR^{jc}R^{kc}, or NR^{jc}C(O)R^{kc};
R^{11c} and R^{12c} together with the carbon atom to which they are attached form a C₃-C₁₂ cycloalkyl or 4- to 12-membered heterocycloalkyl containing 1-4 heteroatoms selected from N, O, and S, wherein the C₃-C₁₂ cycloalkyl or 4- to 12-membered heterocycloalkyl is optionally substituted with one or more of halo, C₁-C₆ alkyl, C₂-C₆ alkenyl, C₂-C₆ alkynyl, hydroxyl, oxo, amino, mono- or di-alkylamino, or C₁-C₆ alkoxyl;
R^{13c} is H, C₁-C₆ alkyl, C₂-C₆ alkenyl, C₂-C₆ alkynyl, C₃-C₁₂ cycloalkyl, or 4- to 12-membered heterocycloalkyl containing 1-4 heteroatoms selected from N, O, and S; and
each of R¹⁴ and R^{15c}, independently, is H, halo, cyano, C₁-C₆ alkyl optionally substituted with one or more of halo or cyano, C₂-C₆ alkenyl optionally substituted with one or more of halo or cyano, C₂-C₆ alkynyl optionally substituted with one or more of halo or cyano, C₃-C₈ cycloalkyl optionally substituted with one or more of halo or cyano, or —OR^{6c}.

In some embodiments, the compound is of Formula (I′″), a tautomer thereof, or a pharmaceutically acceptable salt of the compound or the tautomer.

In some embodiments, when X^{1c} is N, X^{2c} is CH, X^{3c} is N, X^{4c} is CCH₃, X^{5c} is CH, X^{6c} is CH, R^{1c} is H, R^{7c} is

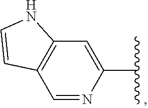 [SMILES: *c1cc2[nH]ccc2cn1]

one of R^{8c} and R^{9c} is H and the other one is CH₃, and R^{14c} is OCH₃, then
R^{15c} is H, halo, cyano, C₁-C₆ alkyl optionally substituted with one or more of halo or cyano, C₂-C₆ alkenyl optionally substituted with one or more of halo or cyano, C₂-C₆ alkynyl optionally substituted with one or more of halo or cyano, C₃-C₈ cycloalkyl optionally substituted with one or more of halo or cyano, or —OR^{6c}.

In some embodiments, when X^{1c} is N, X^{2c} is CH, X^{3c} is N, X^{4c} is CCH₃, X^{5c} is CH, X^{6c} is CH, R^{1c} is H, R^{7c} is

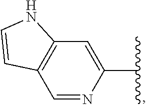 [SMILES: *c1cc2[nH]ccc2cn1]

of R^{8c} and R^{9c} is H and the other one is CH₃, and R^{14c} is OCH₃, then
R^{15c} is H, Cl, Br, cyano, C₁-C₆ alkyl optionally substituted with one or more of halo or cyano, C₂-C₆ alkenyl optionally substituted with one or more of halo or cyano, C₂-C₆ alkynyl optionally substituted with one or more of halo or cyano, C₃-C₈ cycloalkyl optionally substituted with one or more of halo or cyano, or —OR^{6c}.

In some embodiments, wherein when X^{1c} is N, X^{2c} is CH, X^{3c} is N, X^{4C} is CCH₃, X^{5c} is CH, X^{6c} is CH, R^{1c} is H, R^{7c} is selected from the group consisting of

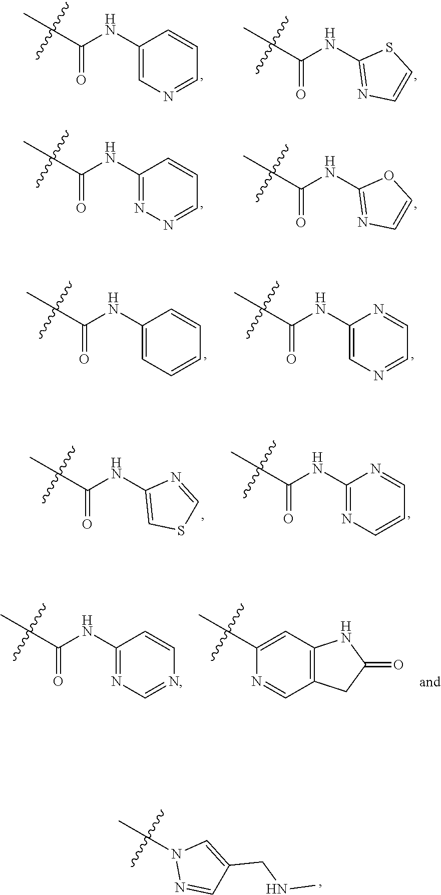

one of R^{8c} and R^{9c} is H and the other one is CH₃, and R^{14c} is Cl, then
R^{15c} is H, halo, cyano, C₁-C₆ alkyl optionally substituted with one or more of halo or cyano, C₂-C₆ alkenyl optionally substituted with one or more of halo or cyano, C₂-C₆ alkynyl optionally substituted with one or more of halo or cyano, C₃-C₈ cycloalkyl optionally substituted with one or more of halo or cyano, or —OR^{6c}.

In some embodiments, wherein when X^{1c} is N, X^{2c} is CH, X^{3c} is N, Xc is CCH₃, X^{5c} is CH, X^{6c} is CH, R^{1c} is H, R^{7c} is selected from the group consisting of

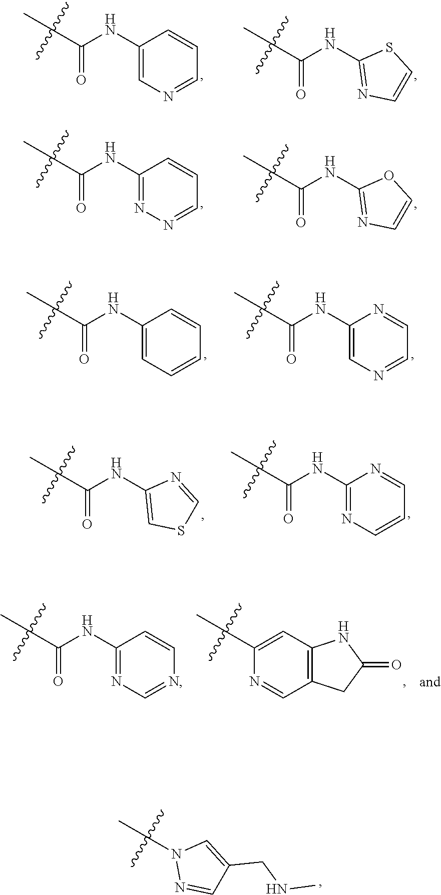

one of R^{8c} and R^{9c} is H and the other one is CH₃, and R^{14C} is Cl, then
R^{15c} is halo, cyano, C₁-C₆ alkyl optionally substituted with one or more of halo or cyano, C₂-C₆ alkenyl optionally substituted with one or more of halo or cyano, C₂-C₆ alkynyl optionally substituted with one or more of halo or cyano, C₃-C₈ cycloalkyl optionally substituted with one or more of halo or cyano, or —OR⁶.

In some embodiments, the compound is not one of the following compounds:

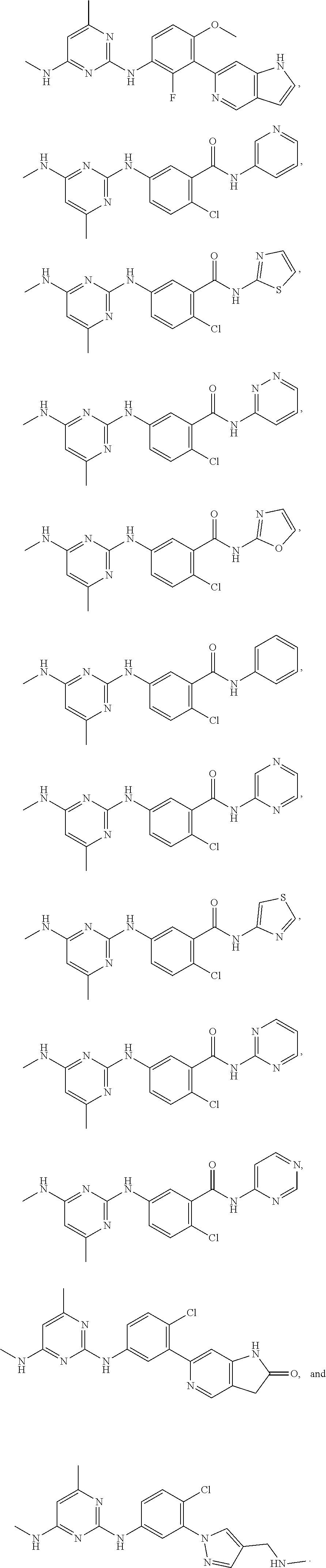

In some embodiments, the compound is of Formula (II′″) or a tautomer thereof, or a pharmaceutically acceptable salt of the compound or the tautomer.

In some embodiments, when X^{5c} is CH, X^{7c} is CH, R^{7c} is

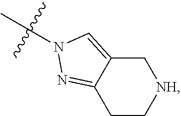 [SMILES: *n1cc2c(n1)CCNC2]

one of R^{8c} and R^{9c} is H and the other one is CH₃, R^{10c} is

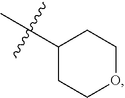 [SMILES: *C1CCOCC1]

and R^{14c} is OCH₃, then
R^{15c} is H, halo, cyano, C₁-C₆ alkyl optionally substituted with one or more of halo or cyano, C₂-C₆ alkenyl optionally substituted with one or more of halo or cyano, C₂-C₆ alkynyl optionally substituted with one or more of halo or cyano, C₃-C₈ cycloalkyl optionally substituted with one or more of halo or cyano, or —OR^{6c}.

In some embodiments, when X^{5c} is CH, X^{7c} is CH, R^{7c} is

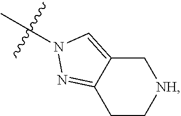 [SMILES: *n1cc2c(n1)CCNC2]

one of R^{8c} and R^{9c} is H and the other one is CH₃, R^{10c} is

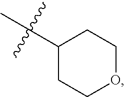 [SMILES: *C1CCOCC1]

and R^{14c} is OCH₃, then

R^{15c} is H, Cl, Br, cyano, C₁-C₆ alkyl optionally substituted with one or more of halo or cyano, C₂-C₆ alkenyl optionally substituted with one or more of halo or cyano, C₂-C₆ alkynyl optionally substituted with one or more of halo or cyano, C₃-C₈ cycloalkyl optionally substituted with one or more of halo or cyano, or —OR⁶.

In some embodiments, the compound is not

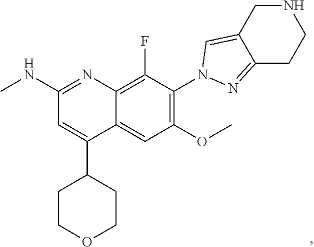 [SMILES: CNc1cc(C2CCOCC2)c2cc(OC)c(-n3cc4c(n3)CCNC4)c(F)c2n1]

In some embodiments, the compound is of Formula (III′″) or a tautomer thereof, or a pharmaceutically acceptable salt of the compound or the tautomer.

In some embodiments, when X^{5c} is CH, X^{8c} is CR^{11c}R^{12c}, in which R^{11c} and R^{12c} together with the carbon atom to which they are attached form a cyclobutyl, R^{7c} is

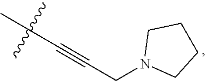 [SMILES: *C#CCN1CCCC1]

one of R^{8c} and R^{9c} is H and the other one is CH₃, and R^{14c} is OCH₃, then
R^{15c} is H, halo, cyano, C₁-C₆ alkyl optionally substituted with one or more of halo or cyano, C₂-C₆ alkenyl optionally substituted with one or more of halo or cyano, C₂-C₆ alkynyl optionally substituted with one or more of halo or cyano, C₃-C₈ cycloalkyl optionally substituted with one or more of halo or cyano, or —OR^{6c}.

In some embodiments, when X^{5c} is CH, X^{8c} is CR^{11c}R^{12c}, in which R^{11c} and R^{12c} to ether with the carbon atom to which they are attached form a cyclobutyl, R^{7c} is

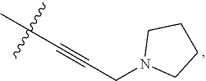 [SMILES: *C#CCN1CCCC1]

one of R^{8c} and R^{9c} is H and the other one is CH₃, and R^{14c} is OCH₃, then
R^{15c} is H, Cl, Br, cyano, C₁-C₆ alkyl optionally substituted with one or more of halo or cyano, C₂-C₆ alkenyl optionally substituted with one or more of halo or cyano, C₂-C₆ alkynyl optionally substituted with one or more of halo or cyano, C₃-C₈ cycloalkyl optionally substituted with one or more of halo or cyano, or —OR^{6c}.

In some embodiments, the compound is not

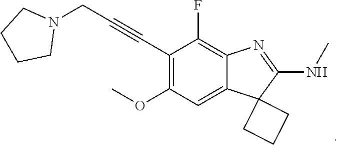 [SMILES: CNC1=Nc2c(cc(OC)c(C#CCN3CCCC3)c2F)C12CCC2]

In some embodiments, at least one of R^{14c} and R^{15c} is halo. In some embodiments, at least one of R^{14c} and R^{15c} is F. In some embodiments, at least one of R^{14c} and R^{15c} is Cl. In some embodiments, at least one of R^{14c} and R^{15c} is Br. In some embodiments, one of R^{14c} and R^{15c} is halo. In some embodiments, one of R^{14c} and R^{15c} is F. In some embodiments, one of R^{14c} and R^{15c} is Cl. In some embodiments, one of R^{14c} and R^{15c} is Br. In some embodiments, R^{14c} is halo. In some embodiments, R^{14c} is F. In some embodiments, R^{14c} is Cl. In some embodiments, R^{14c} is Br. In some embodiments, R^{15c} is halo. In some embodiments, R^{15c} is F. In some embodiments, R^{15c} is Cl. In some embodiments, R^{15c} is Br. In some embodiments, both of R^{14c} and R^{15c} are halo.

In some embodiments, one of R^{14c} and R^{15c} is halo, and the other one is H, cyano, C₁-C₆ alkyl optionally substituted with one or more of halo or cyano, C₂-C₆ alkenyl optionally substituted with one or more of halo or cyano, C₂-C₆ alkynyl optionally substituted with one or more of halo or cyano, C₃-C₈ cycloalkyl optionally substituted with one or more of halo or cyano, or —OR^{6c}.

In some embodiments, one of R^{14c} and R^{15c} is halo, and the other one is H, C₁-C₆ alkyl optionally substituted with one or more of halo or cyano, C₃-C₈ cycloalkyl optionally substituted with one or more of halo or cyano, or —OR^{6c}, in which R^{6c} is C₁-C₆ alkyl optionally substituted with one or more of halo or cyano.

In some embodiments, one of R^{14c} and R^{15c} is halo, and the other one is H, C₁-C₆ alkyl, C₃-C₈ cycloalkyl, or —OR^{6c}, in which R^{6c} is C₁-C₆ alkyl. In some embodiments, R^{14c} is halo, and R^{15c} is H, C₁-C₆ alkyl, C₃-C₈ cycloalkyl, or —OR, in which R^{6c} is C₁-C₆ alkyl. In some embodiments, R^{14c} is halo, and R^{15c} is H. In some embodiments, R^{14c} is halo, and R^{15c} is C₁-C₆ alkyl. In some embodiments, R^{14c} is halo, and R^{15c} is C₃-C₈ cycloalkyl. In some embodiments, R^{14c} is halo, and R^{15c} is —OR^{6c}, in which R^{6c} is C₁-C₆ alkyl. In some embodiments, R^{15c} is halo, and R^{14c} is H, C₁-C₆ alkyl, C₃-C₈ cycloalkyl, or —OR^{6c}, in which R^{6c} is C₁-C₆ alkyl. In some embodiments, R^{15c} is halo, and R^{14c} is H. In some embodiments, R^{15c} is halo, and R^{14c} is C₁-C₆ alkyl. In some embodiments, R^{15c} is halo, and R^{14c} is C₃-C₈ cycloalkyl. In some embodiments, R^{15c} is halo, and R^{14c} is —OR^{6c}, in which R^{6c} is C₁-C₆ alkyl. In some embodiments, one of R^{14c} and R^{15c} is halo, and the other one is H, —CH₃, cyclopropyl, or —OCH₃.

In some embodiments, the compound is of any of Formula (I′″-1), (I′″-2), (II′″-1), (II′″-2), (III′″-1), or (III′″-2):

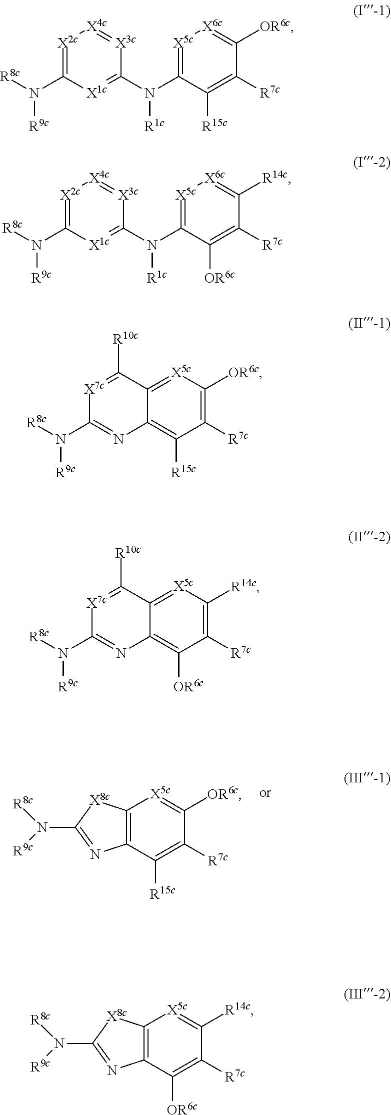

a tautomer thereof, or a pharmaceutically acceptable salt of the compound or the tautomer, wherein
X^{1c} is N or CR^{2c};
X^{2c} is N or CR^{3c};
X^{3c} is N or CR^{4c};
X^{4c} is N or CR^{5c};
each of X^{5c}, X^{6c} and X^{7c} is independently N or CH;
R^{1c} is H or C₁-C₄ alkyl;
each of R^{2c}, R^{3c}, R^{4c}, and R^{5c}, independently is selected from the group consisting of H, halo, cyano, C₁-C₆ alkoxyl, C₆-C₁₀ aryl, OH, NR^{ac}R^{bc}, C(O)NR^{ac}R^{bc}, NR^{ac}C(O)R^{bc}, C(O)OR^{ac}, OC(O)R^{ac}, OC(O)NR^{ac}R^{bc}, NR^{ac}C(O)OR^{bc}, C₃-C₈ cycloalkyl, 4- to 7-membered heterocycloalkyl, 5- to 6-membered heteroaryl, C₁-C₆ alkyl, C₂-C₆ alkenyl, and C₂-C₆ alkynyl, wherein the C₆-C₁₀ aryl, C₃-C₈ cycloalkyl, 4- to 7-membered heterocycloalkyl, 5- to 6-membered heteroaryl, C₁-C₆ alkoxyl, C₁-C₆ alkyl, C₂-C₆ alkenyl, and C₂-C₆ alkynyl, are each optionally substituted with one or more of halo, OR^{ac}, or NR^{ac}R^{bc}, in which each of R^{ac} and R^{bc} independently is H or C₁-C₆ alkyl;
R^{6c} is -Q^{1c}-T^{1c}, in which Q^{1c} is a bond, or C₁-C₆ alkylene, C₂-C₆ alkenylene, or C₂-C₆ alkynylene linker each optionally substituted with one or more of halo, cyano, hydroxyl, oxo, or C₁-C₆ alkoxyl, and T^{1c} is H, halo, cyano, or R^{S1c}, in which R^{S1c} is C₃-C₈ cycloalkyl, phenyl, 4- to 12-membered heterocycloalkyl containing 1-4 heteroatoms selected from N, O, and S, or a 5- or 6-membered heteroaryl and R^{S1c} is optionally substituted with one or more of halo, C₁-C₆ alkyl, C₂-C₆ alkenyl, C₂-C₆ alkynyl, hydroxyl, oxo, —C(O)R^{cc}, —C(O)OR^{cc}, —SO₂R^{cc}, —SO₂N(R^{cc})₂, —NR^{cc}C(O)R^{dc}, —C(O)NR^{cc}R^{dc}, —NR^{cc}C(O)OR^{dc}, —OC(O)NR^{cc}R^{dc}, NR^{cc}R^{dc}, or C₁-C₆ alkoxyl, in which each of R^{cc} and R^{dc} independently is H or C₁-C₆ alkyl;
R^{7c} is -Q^{2c}-T^{2c}, in which Q^{2c} is a bond, a bond or C₁-C₆ alkylene, C₂-C₆ alkenylene, or C₂-C₆ alkynylene linker optionally substituted with one or more of halo, cyano, hydroxyl, amino, mono- or di-alkylamino, and T^{2c} is H, halo, cyano, OR^{ec}, OR^{fc}, C(O)R^{fc}, NR^{ec}R^{fc}, C(O)NR^{ec}R^{fc}, NR^{ec}C(O)R^{fc}, C₆-C₁₀ aryl, 5- to 10-membered heteroaryl, C₃-C₁₂ cycloalkyl, or 4- to 12-membered heterocycloalkyl, and wherein the C₆-C₁₀ aryl, 5- to 10-membered heteroaryl, C₃-C₁₂ cycloalkyl, or 4- to 12-membered heterocycloalkyl is optionally substituted with one or more -Q^{3c}-T^{3c}, wherein each Q^{3c} independently is a bond or C₁-C₃ alkylene linker each optionally substituted with one or more of halo, cyano, hydroxyl, or C₁-C₆ alkoxy, and each T^{3c} independently is selected from the group consisting of H, halo, cyano, C₁-C₆ alkyl, C₂-C₆ alkenyl, C₂-C₆ alkynyl, C₃-C₈ cycloalkyl, C₆-C₁₀ aryl, 4- to 7-membered heterocycloalkyl containing 1-4 heteroatoms selected from N, O, and S, 5- to 6-membered heteroaryl, OR^{ec}, OR^{fc}, C(O)R^{fc}, C(O)OR^{fc}, OC(O)R^{fc}, S(O)₂R^{fc}, NR^{fc}R^{gc}, OC(O)NR^{fc}R^{gc}, NR^{fc}C(O)OR^{gc}, C(O)NR^{fc}R^{gc}, and NR^{fc}C(O)R^{gc}; or -Q^{3c}-T^{3c} is oxo;
each R^{ec} independently is H or C₁-C₆ alkyl optionally substituted with one or more of halo, cyano, hydroxyl, amino, mono- or di-alkylamino, or C₁-C₆ alkoxyl; each of R^{fc} and R^{gc}, independently, is -Q^{6c}-T^{6c}, in which Q^{6c} is a bond or C₁-C₆ alkylene, C₂-C₆ alkenylene, or C₂-C₆ alkynylene linker each optionally substituted with one or more of halo, cyano, hydroxyl, or C₁-C₆ alkoxyl, and T^{6c} is H, halo, OR^{m1c}, NR^{m1c}R^{m2c}, NR^{m1c}C(O)R^{m2c}, C(O)NR^{m1c}R^{m2c}, C(O)R^{m1c}, C(O)OR^{m1c}, NR^{m1c}(O)OR^{m2c}, OC(O)NR^{m1c}R^{m2c}, S(O)₂R^{m1c}, S(O)₂NR^{m1c}R^{m2c}, or R^{S3c}, in which each of R^{m1c} and R^{m2c} independently is H or C₁-C₆ alkyl, and R^{S3c} is C₃-C₈ cycloalkyl, C₆-C₁₀ aryl, 4- to 12-membered heterocycloalkyl containing 1-4 heteroatoms selected from N, O, and S, or a 5- to 10-membered heteroaryl, and R^{S3c} is optionally substituted with one or more -Q^{7c}-T^{7c}, wherein each Q^{7c} independently is a bond or C₁-C₃ alkylene linker each optionally substituted with one or more of halo, cyano, hydroxyl, or C₁-C₆ alkoxy, and each T^{7c} independently is selected from the group consisting of H, halo, cyano, C₁-C₆ alkyl, C₂-C₆ alkenyl, C₂-C₆ alkynyl, C₃-C₈ cycloalkyl, C₆-C₁₀ aryl, 4- to 7-membered heterocycloalkyl containing 1-4 heteroatoms selected from N, O, and S, 5- to 6-membered heteroaryl, OR^{n1c}, C(O)R^{n1c}, C(O)OR^{n1c}, OC(O)R^{n1c}, S(O)₂R^{n1c}, NR^{n1c}R^{n2c}, OC(O)NR^{n1c}R^{n2c}, NR^{n1c}C(O)OR^{n2c}, C(O)NR^{n1c}R^{n2c}, and NR^{n1c}C(O)R^{n2c}, each of R^{n1c} and R^{n2c} independently being H or C₁-C₆ alkyl; or Q^{7c}-T^{7c} is oxo; R^{8c} is H or C₁-C₆ alkyl;
R^{9c} is -Q^{4c}-T^{4c}, in which Q^{4c} is a bond or C₁-C₆ alkylene, C₂-C₆ alkenylene, or C₂-C₆ alkynylene linker each optionally substituted with one or more of halo, cyano, hydroxyl, or C₁-C₆ alkoxyl, and T^{4c} is H, halo, OR^{hc}, NR^{hc}R^{ic}, NR^{hc}C(O)R^{ic}, C(O)NR^{hc}R^{ic}, C(O)R^{hc}, C(O)OR^{hc}, NR^{hc}C(O)OR^{ic}, OC(O)NR^{hc}R^{ic}, S(O)₂R^{hc}, S(O)₂NR^{hc}R^{ic}, or R^{S2c}, in which each of R^{hc} and R^{ic} independently is H or C₁-C₆ alkyl, and R^{S2c} is C₃-C₈ cycloalkyl, C₆-C₁₀ aryl, 4- to 12-membered heterocycloalkyl containing 1-4 heteroatoms selected from N, O, and S, or a 5- to 10-membered heteroaryl, and R^{S2c} is optionally substituted with one or more -Q^{5c}-T^{5c}, wherein each Q^{5c} independently is a bond or C₁-C₃ alkylene linker each optionally substituted with one or more of halo, cyano, hydroxyl, or C₁-C₆ alkoxy, and each T^{5c} independently is selected from the group consisting of H, halo, cyano, C₁-C₆ alkyl, C₂-C₆ alkenyl, C₂-C₆ alkynyl, C₃-C₈ cycloalkyl, C₆-C₁₀ aryl, 4- to 7-membered heterocycloalkyl containing 1-4 heteroatoms selected from N, O, and S, 5- to 6-membered heteroaryl, OR^{jc}, C(O)R^{jc}, C(O)OR^{jc}, OC(O)R^{jc}, S(O)₂R^{jc}, NR^{jc}R^{kc}, OC(O)NR^{jc}R^{kc}, NR^{jc}C(O)OR^{kc}, C(O)NR^{jc}R^{kc}, and NR^{jc}C(O)R^{kc}, each of R^{jc} and R^{kc} independently being H or C₁-C₆ alkyl; or Q^{5c}-T^{5c} is oxo;
R¹⁰ is halo, C₁-C₆ alkyl, C₂-C₆ alkenyl, C₂-C₆ alkynyl, C₃-C₈ cycloalkyl, or 4- to 12-membered heterocycloalkyl containing 1-4 heteroatoms selected from N, O, and S, wherein each of the C₁-C₆ alkyl, C₂-C₆ alkenyl, C₂-C₆ alkynyl, C₃-C₈ cycloalkyl, and 4- to 12-membered heterocycloalkyl is optionally substituted with one or more halo, cyano, hydroxyl, oxo, amino, mono- or di-alkylamino, C₁-C₆ alkyl, C₂-C₆ alkenyl, C₂-C₆ alkynyl, C₁-C₆ alkoxy, C(O)NR^{jc}R^{kc}, or NR^{jc}C(O)R^{kc}; and
R^{11c} and R^{12c} together with the carbon atom to which they are attached form a C₃-C₁₂ cycloalkyl or 4- to 12-membered heterocycloalkyl containing 1-4 heteroatoms selected from N, O, and S, wherein the C₃-C₁₂ cycloalkyl or 4- to 12-membered heterocycloalkyl is optionally substituted with one or more of halo, C₁-C₆ alkyl, C₂-C₆ alkenyl, C₂-C₆ alkynyl, hydroxyl, oxo, amino, mono- or di-alkylamino, or C₁-C₆ alkoxyl
each of R^{14c} and R^{15c}, independently, is H, halo, cyano, C₁-C₆ alkyl optionally substituted with one or more of halo or cyano, C₂-C₆ alkenyl optionally substituted with one or more of halo or cyano, C₂-C₆ alkynyl optionally substituted with one or more of halo or cyano, or C₃-C₈ cycloalkyl optionally substituted with one or more of halo or cyano.

In some embodiments, the compound is of Formula (I′″-1) or (I′″-2), a tautomer thereof, or a pharmaceutically acceptable salt of the compound or the tautomer.

In some embodiments, at least one of X^{1c}, X^{2c}, X^{3c} and X^{4c} is N. In some embodiments, X^{1c} and X^{3c} are N. In some embodiments, X^{1c} and X^{3c} are N, X^{2c} is CR^{3C} and X^{4c} is CR^{5c}.

In some embodiments,

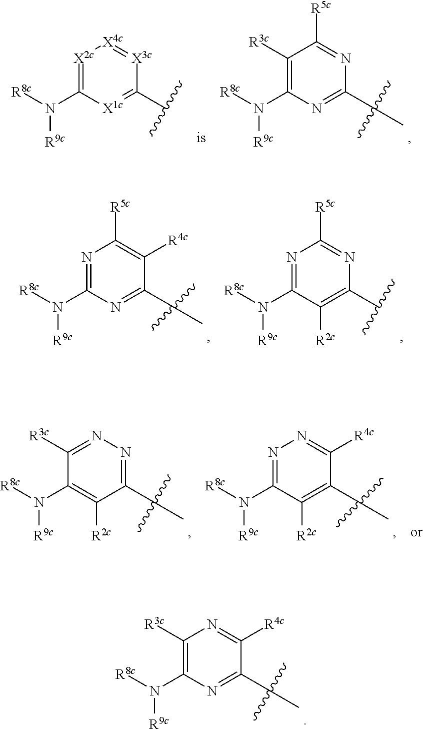

In some embodiments,

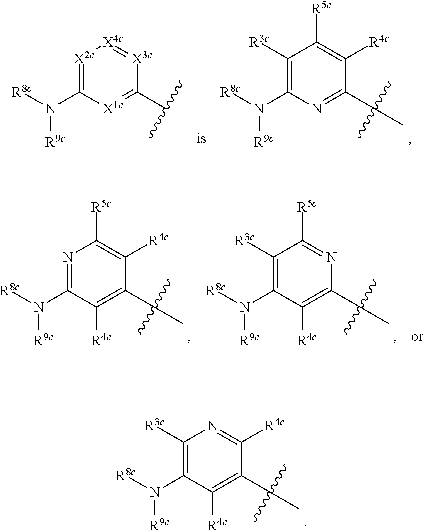

In some embodiments, the compound is of Formula (I′″-1a), (I′″-2a), (I′″-1b), (I′″-2b), (I′″-1c), or (I′″-2c):

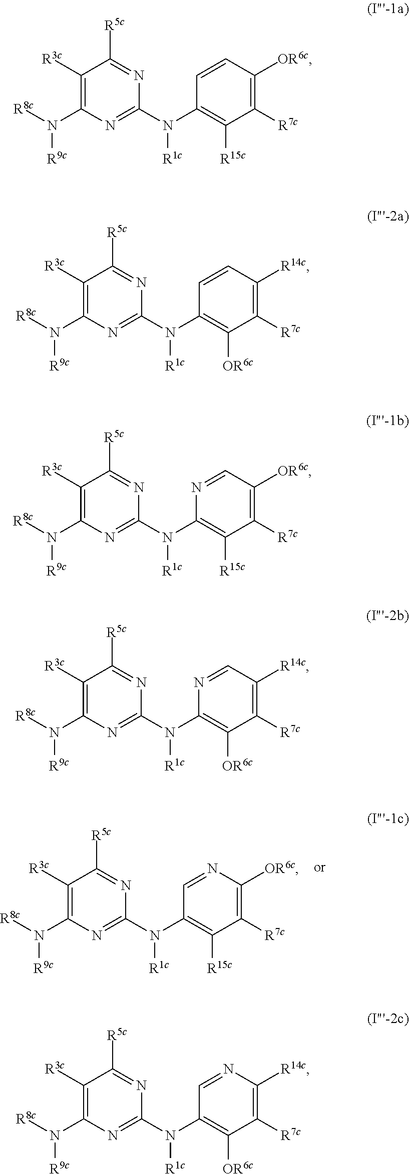

a tautomer thereof, or a pharmaceutically acceptable salt of the compound or the tautomer.

In some embodiments, at most one of R^{3c} and R^{5c} is not H. In some embodiments, at least one of R^{3c} and R^{5c} is not H. In some embodiments, R^{3c} is H or halo.

In some embodiments, the compound is of Formula (I′″-1d), (I′″-2d), (I′″-1e), (I′″-2e), (I′″-1f), or (I′″-2f):

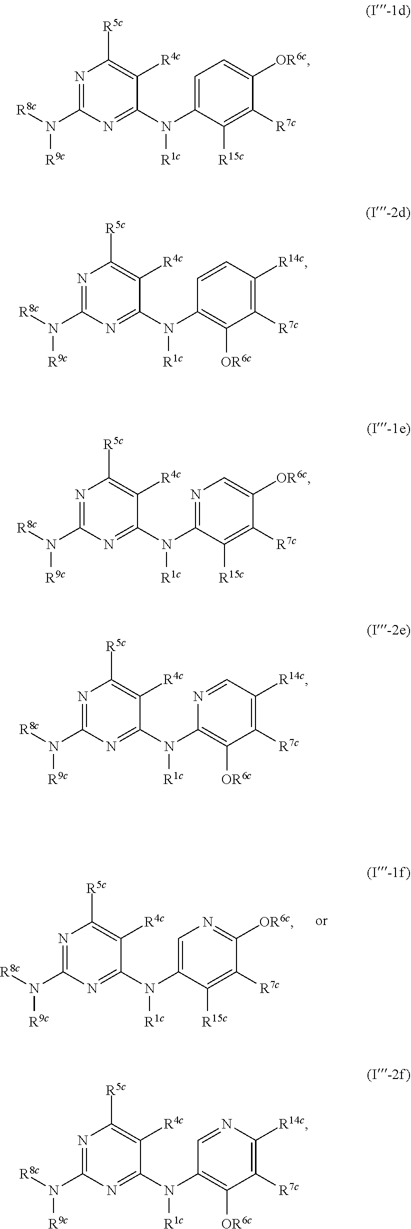

a tautomer thereof, or a pharmaceutically acceptable salt of the compound or the tautomer.

In some embodiments, at most one of R^{4c} and R^{5c} is not H. In some embodiments, at least one of R^{4c} and R^{5c} is not H. In some embodiments, R^{4c} is H, C₁-C₆ alkyl, or halo.

In some embodiments, the compound of Formula (I′″-1g), (I′″-2g), (I′″-1h), (I′″-2h), (I′″-1i), or (I′″-2i):

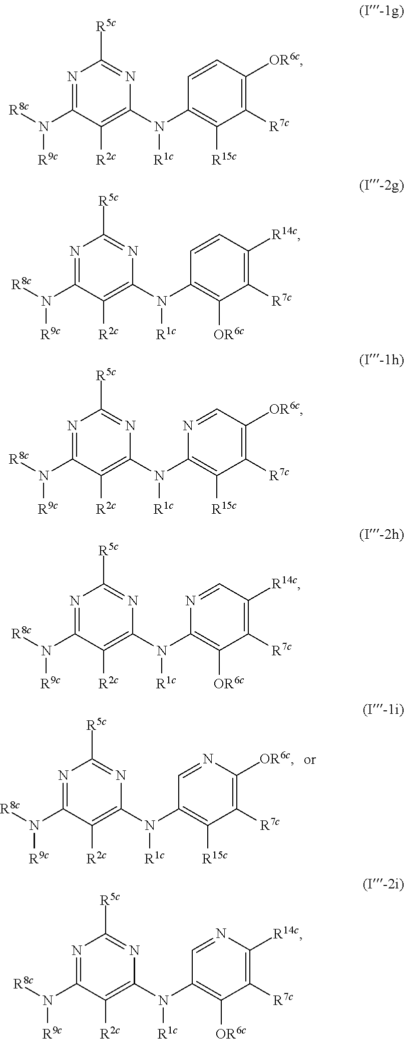

a tautomer thereof, or a pharmaceutically acceptable salt of the compound or the tautomer.

In some embodiments, at most one of R^{2c} and R^{5c} is not H. In some embodiments, at least one of R^{2c} and R^{5c} is not H. In some embodiments, R^{2c} is H, C₁-C₆ alkyl, or halo. In some embodiments, R^{5c} is C₁-C₆ alkyl.

In some embodiments, the compound is of Formula (II′″-1) of (I′″-2), a tautomer thereof, or a pharmaceutically acceptable salt of the compound or the tautomer.

In some embodiments, each of X^{5c}, X^{6c} and X^{7c} is CH. In some embodiments, at least one of X^{5c}, X^{6c} and X^{7c} is N. In some embodiments, at most one of X^{5c}, X^{6c} and X^{7c} is N.

In some embodiments, R¹⁰ is optionally substituted 4- to 7-membered heterocycloalkyl containing 1-4 heteroatoms selected from N, O, and S. In some embodiments, R¹⁰ is connected to the bicyclic group of Formula (II′″-1) or (II′″-2) via a carbon-carbon bond. In some embodiments, R¹⁰ is connected to the bicyclic group of Formula (II′″-1) or (II′″-2) via a carbon-nitrogen bond.

In some embodiments, the compound is of Formula (III′″-1) or (III′″-2), a tautomer thereof, or a pharmaceutically acceptable salt of the compound or the tautomer.

In some embodiments, R^{11c} and R^{12c} together with the carbon atom to which they are attached form a 4- to 7-membered heterocycloalkyl containing 1-4 heteroatoms selected from N, O, and S, wherein the 4- to 7-membered heterocycloalkyl is optionally substituted with one or more of halo, C₁-C₆ alkyl, hydroxyl, oxo, amino, mono- or di-alkylamino, or C₁-C₆ alkoxyl.

In some embodiments, R^{11c} and R^{12c} together with the carbon atom to which they are attached form a C₄-C₈ cycloalkyl which is optionally substituted with one or more of halo, C₁-C₆ alkyl, hydroxyl, oxo, amino, mono- or di-alkylamino, or C₁-C₆ alkoxyl.

In some embodiments, each of X^{5c} and X^{6c} is CH. In some embodiments, each of Xc and X^{6c} is N. In some embodiments, one of X^{5c} and X^{6c} is CH and the other is CH.

In some embodiments, R^{6c} is -Q^{1c}-T^{1c}, in which Q^{1c} is a bond or C₁-C₆ alkylene linker optionally substituted with one or more of halo, and T^{1c} is H, halo, cyano, or R^{S1c}, in which R^{S1c} is C₃-C₈ cycloalkyl, phenyl, 4- to 12-membered heterocycloalkyl containing 1-4 heteroatoms selected from N, O, and S, or a 5- or 6-membered heteroaryl and R^{S1c} is optionally substituted with one or more of halo, C₁-C₆ alkyl, hydroxyl, oxo, NR^{cc}R^{dd}, or C₁-C₆ alkoxyl.

In some embodiments, wherein R^{6c} is C₁-C₆ alkyl optionally substituted with one or more of halo, cyano, hydroxyl, or C₁-C₆ alkoxyl. In some embodiments, R^{6c} is C₁-C₆ alkyl. In some embodiments, R^{6c} is —CH₃.

In some embodiments, R^{7c} is -Q^{2c}-T^{2c}, in which Q^{2c} is a bond or C₁-C₆ alkylene, C₂-C₆ alkenylene, or C₂-C₆ alkynylene linker optionally substituted with one or more of halo, cyano, hydroxyl, amino, mono- or di-alkylamino, and T^{2c} is C(O)NR^{ec}R^{fc}.

In some embodiments, Q^{2c} is a bond. In some embodiments, R^{ec} is H.

In some embodiments, R^{fc} is -Q^{6c}-T^{6c}, in which Q^{6c} is a bond or C₁-C₆ alkylene, C₂-C₆ alkenylene, or C₂-C₆ alkynylene linker each optionally substituted with one or more of halo, cyano, hydroxyl, or C₁-C₆ alkoxyl, and T^{6c} is H, NR^{m1c}R^{m2c}, or R^{S3c}, in which each of R^{m1c} and R^{m2c} independently is H, C₁-C₆ alkyl, or —(C₁-C₆ alkyl)-R^{S3c}, and R^{S3c} is C₃-C₈ cycloalkyl, C₆-C₁₀ aryl, 4- to 12-membered heterocycloalkyl containing 1-4 heteroatoms selected from N, O, and S, or a 5- to 10-membered heteroaryl, and R^{S3}c is optionally substituted with one or more -Q^{7c}-T^{7c}.

In some embodiments, R^{fc} is -Q^{6c}-T^{6c}, in which Q^{6c} is a bond or C₁-C₆ alkylene, C₂-C₆ alkenylene, or C₂-C₆ alkynylene linker each optionally substituted with one or more of halo, cyano, hydroxyl, or C₁-C₆ alkoxyl, and T^{6c} is H, NR^{m1c}CR^{m2c}, or R^{S3c}, in which each of R^{m1c} and R^{m2c} independently is H or C₁-C₆ alkyl, and R^{S3c} is C₃-C₈ cycloalkyl, C₆-C₁₀ aryl, 4- to 12-membered heterocycloalkyl containing 1-4 heteroatoms selected from N, O, and S, or a 5- to 10-membered heteroaryl, and R^{S3c} is optionally substituted with one or more -Q^{7c}-T^{7c}.

In some embodiments, T^{6c} is 8- to 12-membered bicyclic heterocycloalkyl that comprises a 5- or 6-membered aryl or heteroaryl ring fused with a non-aromatic ring. In some embodiments, T^{6c} is 8- to 12-membered bicyclic heterocycloalkyl that comprises a 5- or 6-membered aryl or heteroaryl ring fused with a non-aromatic ring, in which the 5- or 6-membered aryl or heteroaryl ring is connected to Q^{2c}. In some embodiments, T^{6c} is 5- to 10-membered heteroaryl.

In some embodiments, T^{6c} is selected from

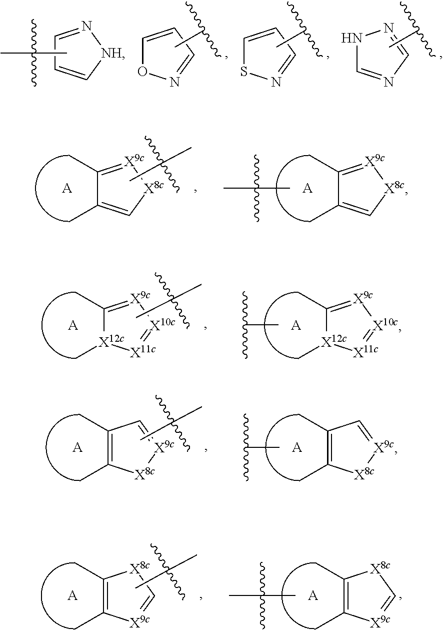

and tautomers thereof, each of which is optionally substituted with one or more -Q^{7c}-T^{7c}, wherein X^{8c} is NH, O, or S, each of X^{9c}, X^{10c}, X^{11c}, and X^{12c} is independently CH or N, and at least one of X^{9c}, X¹⁰, X^{11c}, and X^{12c} is N, and ring A is a C₅-C₈cycloalkyl, phenyl, 6-membered heteroaryl, or 4- to 8-membered heterocycloalkyl containing 1-4 heteroatoms selected from N O and S.

In some embodiments, T^{6c} is selected from

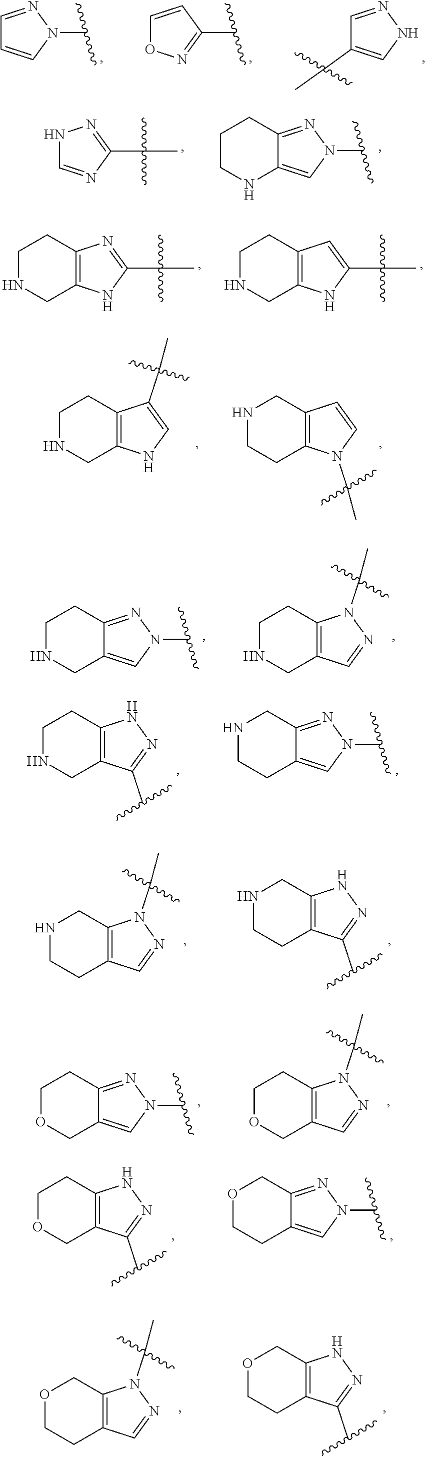

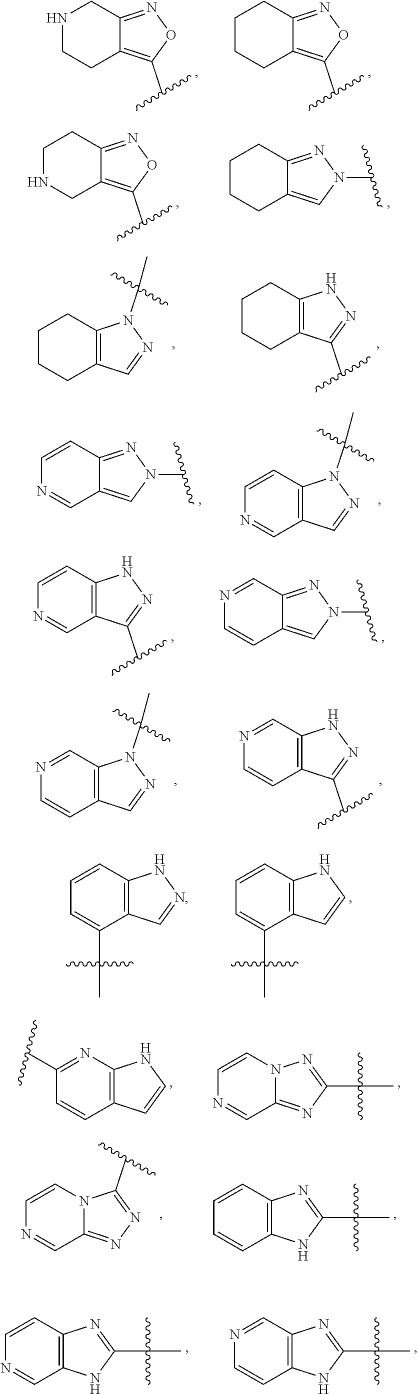

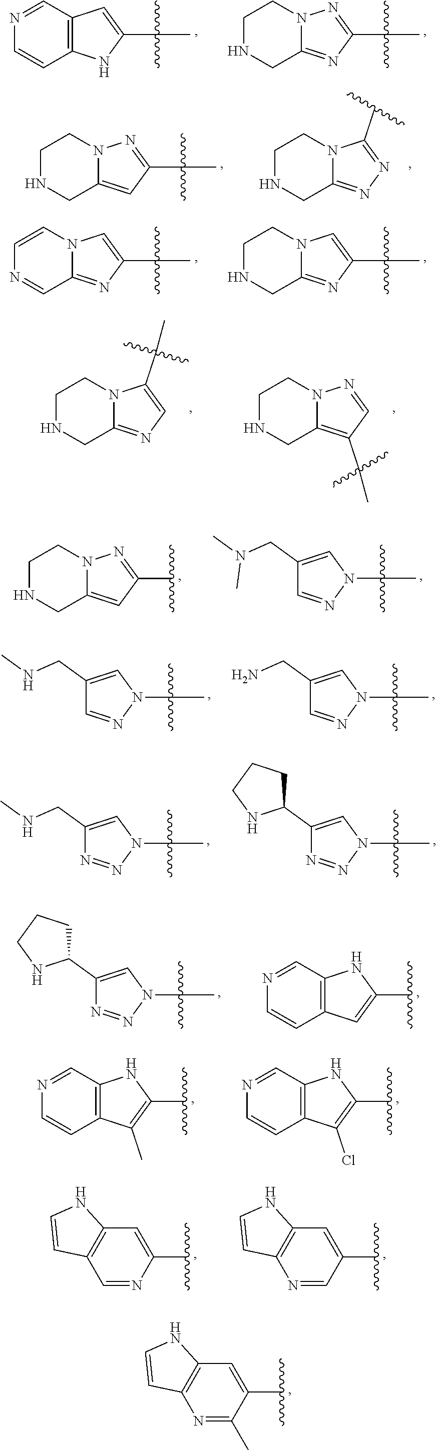

and tautomers thereof, each of which is optionally substituted with one or more -Q^{7c}-T^{7c}.

In some embodiments, each Q^{7c} independently is a bond or C₁-C₃ alkylene linker each optionally substituted with one or more of halo, cyano, hydroxyl, or C₁-C₆ alkoxy, and each T^{7c} independently is selected the group consisting of H, halo, cyano, C₁-C₆ alkyl, C₂-C₆ alkenyl, C₂-C₆ alkynyl, C₃-C₈ cycloalkyl, C₆-C₁₀ aryl, 4- to 7-membered heterocycloalkyl containing 1-4 heteroatoms selected from N, O, and S, 5- to 6-membered heteroaryl, OR^{n1c}, C(O)R^{n1c}, C(O)OR^{n1c}, OC(O)R^{n1c}, S(O)₂R^{n1c}, NR^{n1c}R^{n2c}, OC(O)NR^{n1c}R^{n2c}, NR^{n1c}C(O)OR^{n2c}, C(O)NR^{n1c}R^{n2c}, and NR^{n1c}C(O)R^{n2c}, each of R^{n1c} and R^{n2c} independently being H or C₁-C₆ alkyl; or -Q^{7c}-T^{7c} is oxo.

In some embodiments, each Q^{7c} independently is a bond or C₁-C₃ alkylene linker each optionally substituted with one or more of halo, cyano, hydroxyl, or C₁-C₆ alkoxy, and each T^{7c} independently is selected from the group consisting of H, halo, cyano, C₁-C₆ alkyl, and NR^{n1c}R^{n2c}, each of R^{n1c} and R^{n2c} independently being H or C₁-C₆ alkyl.

In some embodiments, R^{7c} is

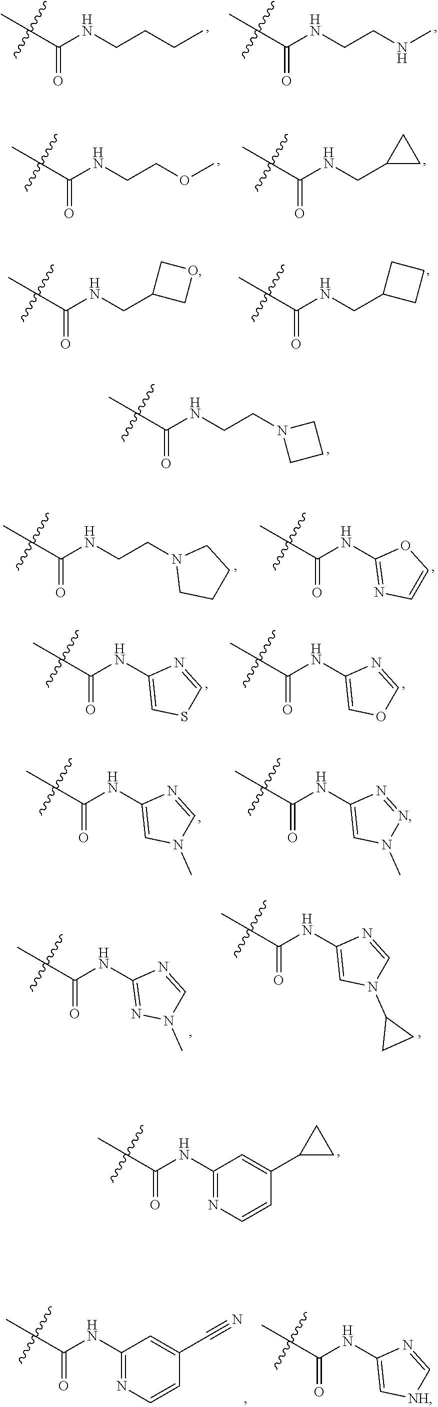

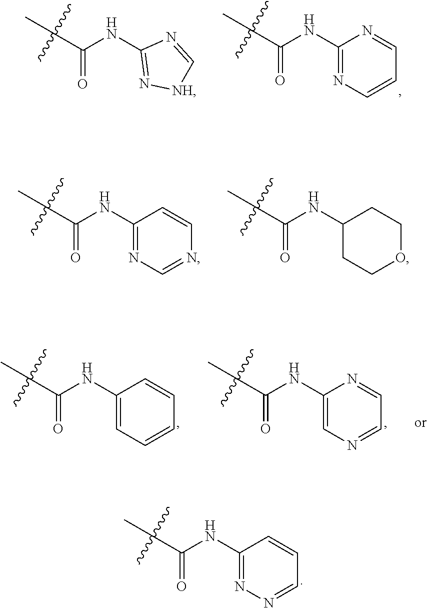

In some embodiments, R^{7c} is -Q^{2c}-T^{2c}, in which Q^{2c} is a bond or C₁-C₆ alkylene, C₂-C₆ alkenylene, or C₂-C₆ alkynylene linker optionally substituted with one or more of halo, cyano, hydroxyl, amino, mono- or di-alkylamino, or C₁-C₆ alkoxyl, and each T^{2c} independently is H, OR^{fc}, OR^{fc}, NR^{cc}R^{fc}, C₃-C₁₂ cycloalkyl, or 4- to 12-membered heterocycloalkyl.

In some embodiments, R^{7c} is

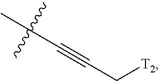

wherein T^{2c} is H, halo, cyano, OR^{cc}, OR^{fc}, C(O)R^{fc}, NR^{cc}R^{fc}, C(O)NR^{cc}R^{fc}, NR^{cc}C(O)R^{fc}, C₆-C₁₀ aryl, 5- to 10-membered heteroaryl, C₃-C₁₂ cycloalkyl, or 4- to 12-membered heterocycloalkyl containing 1-4 heteroatoms selected from N, O, and S, and wherein the C₆-C₁₀ aryl, 5- to 10-membered heteroaryl, C₃-C₁₂ cycloalkyl or 4- to 12-membered heterocycloalkyl is optionally substituted with one or more of halo, hydroxyl, cyano, C₁-C₆ haloalkyl, —SO₂R^{cc}, C₁-C₆ alkoxyl or C₁-C₆ alkyl optionally substituted with one or more of NR^{cc}R^{dc}.

In some embodiments, R^{7c} is

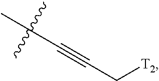

wherein T^{2c} is 5- to 10-membered heteroaryl or 4- to 12-membered heterocycloalkyl optionally substituted with one or more of halo, hydroxyl, C₁-C₆ alkoxyl or C₁-C₆ alkyl.

In some embodiments, R^{7c} is

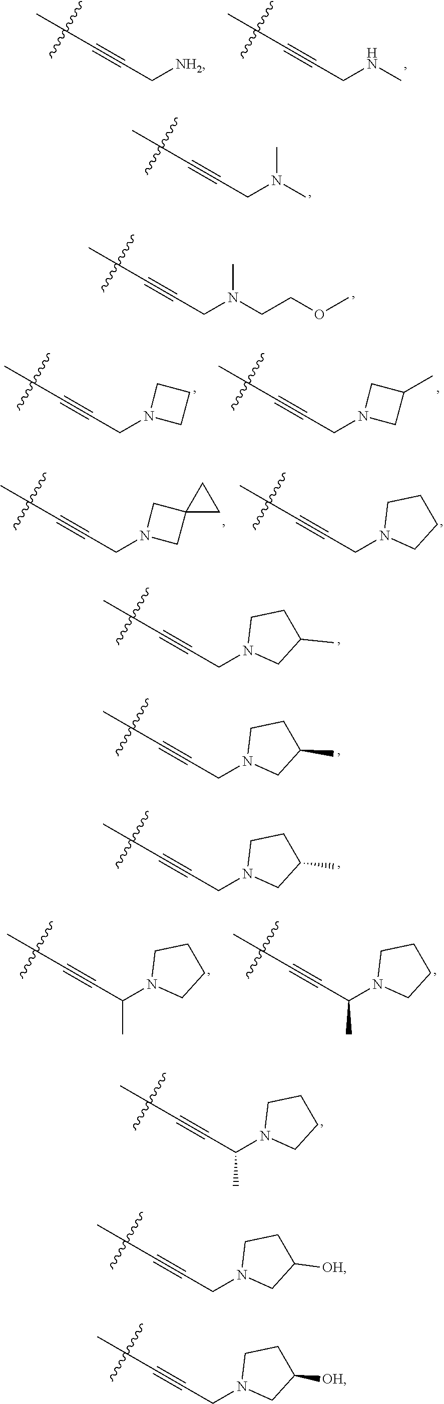

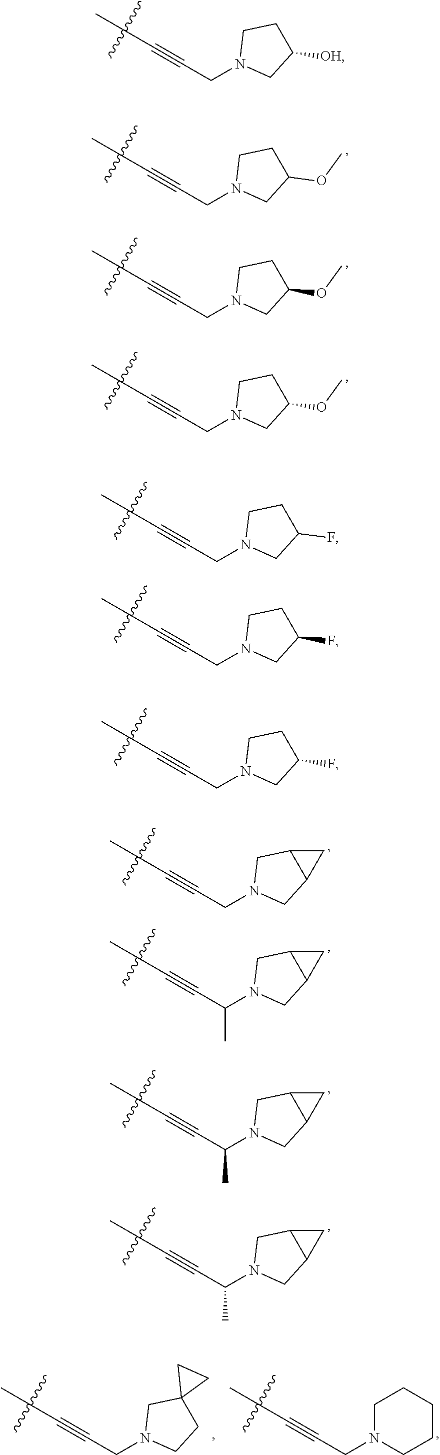

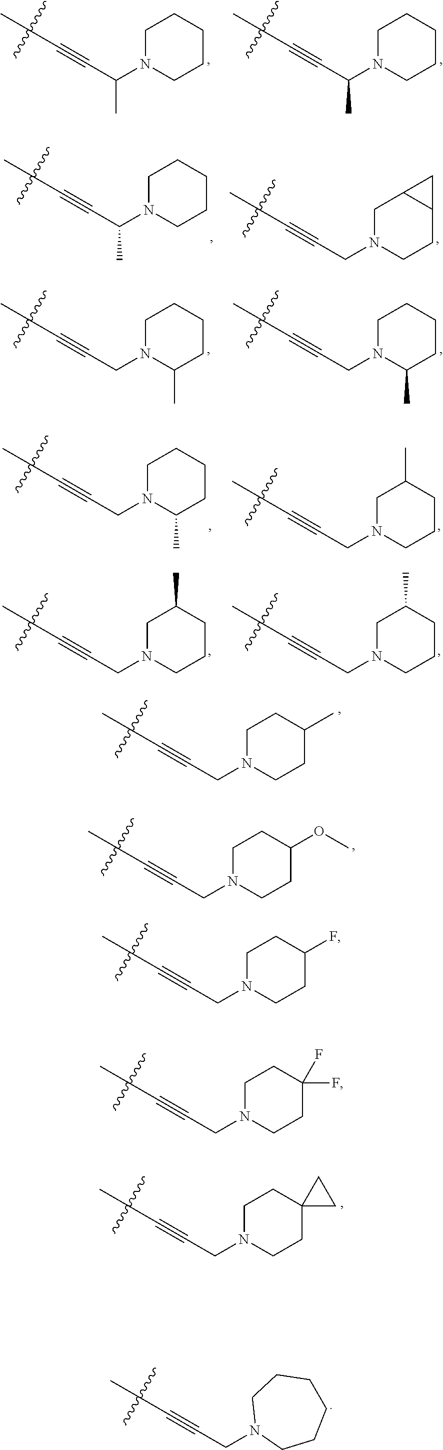

In some embodiments, R^{7c} is OR^{cc}.

In some embodiments, R^{7c} is OR^{fc}.

In some embodiments, R^{7c} is O-Q^{6c}-NR^{m1c}R^{m2c}. In some embodiments, R^{7c} is O-Q^{6c}-NH—(C₁-C₆ alkyl)-R^{S3c}.

In some embodiments, R^{7c} is —CH₂-T^{2c}, wherein T^{2c} is H, halo, cyano, OR^{cc}, OR^{fc}, C(O)R^{fc}, NR^{7c}R^{fc}, C(O)NR^{cc}R^{fc}, NR^{cc}C(O)R^{fc}, C₆-C₁₀ aryl, 5- to 10-membered heteroaryl, C₃-C₁₂ cycloalkyl, or 4- to 12-membered heterocycloalkyl containing 1-4 heteroatoms selected from N, O, and S, and wherein the C₆-C₁₀ aryl, 5- to 10-membered heteroaryl, C₃-C₁₂ cycloalkyl or 4- to 12-membered heterocycloalkyl is optionally substituted with one or more of halo, hydroxyl, cyano, C₁-C₆ haloalkyl, —SO₂R^{cc}, C₁-C₆ alkoxyl or C₁-C₆ alkyl optionally substituted with one or more of NR^{cc}R^{dc}.

In some embodiments, R^{7c} is —CH₂—OR₈.

In some embodiments, R^{7c} is —CH₂—NR₇R₈.

In some embodiments, R^{7c} is

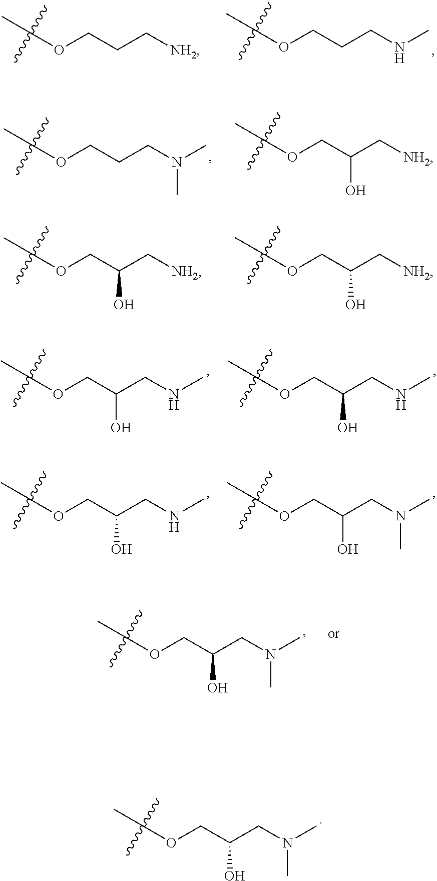

In some embodiments, R^{7c} is

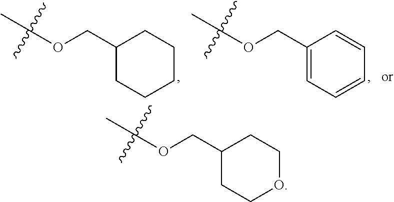

In some embodiments, R^{7c} is

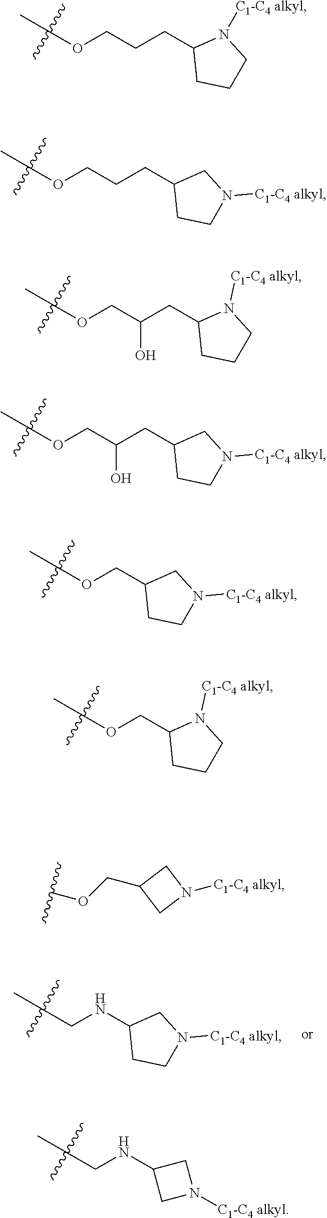

In some embodiments, R^{7c} is

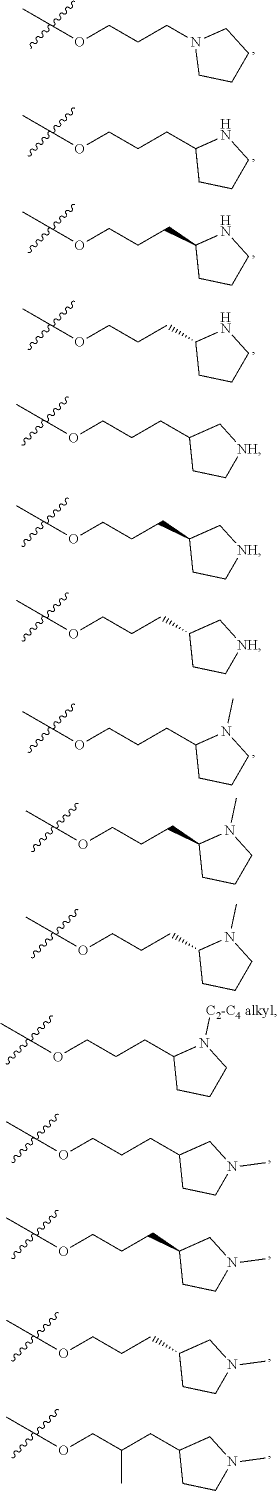

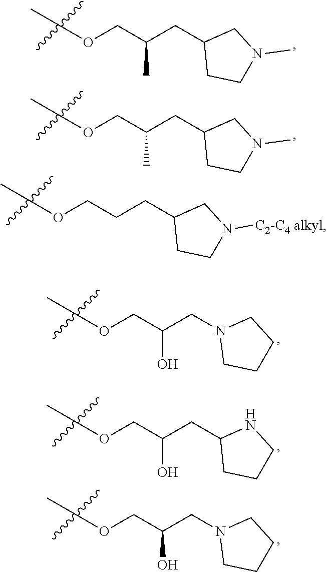

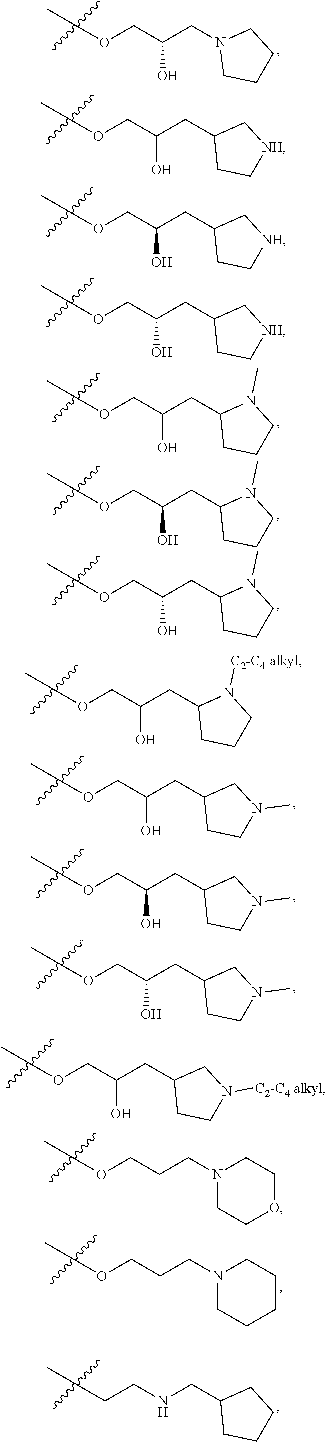

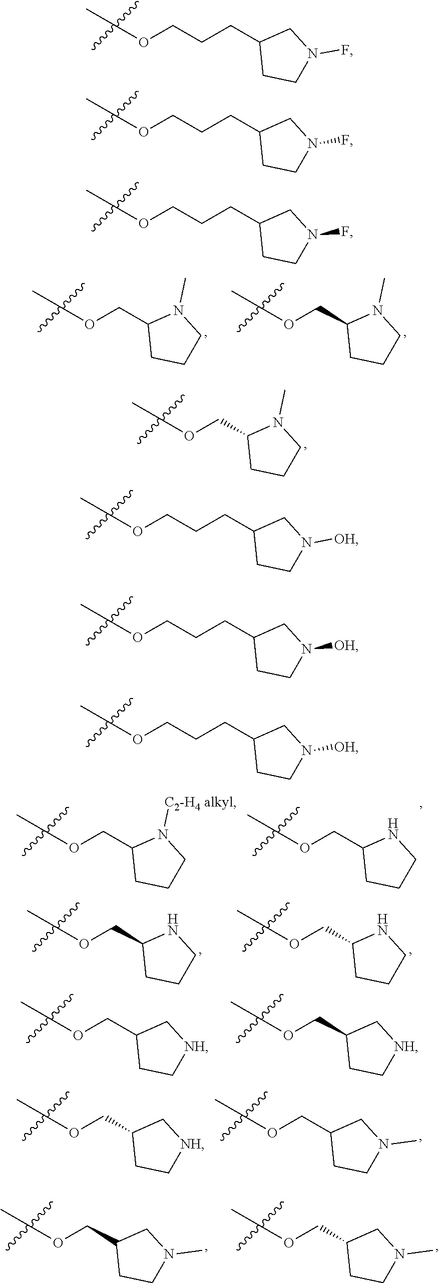

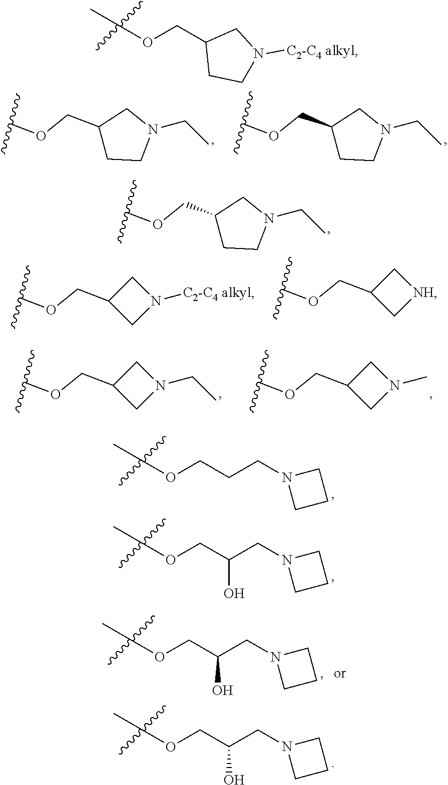

In some embodiments, R^{7c} is

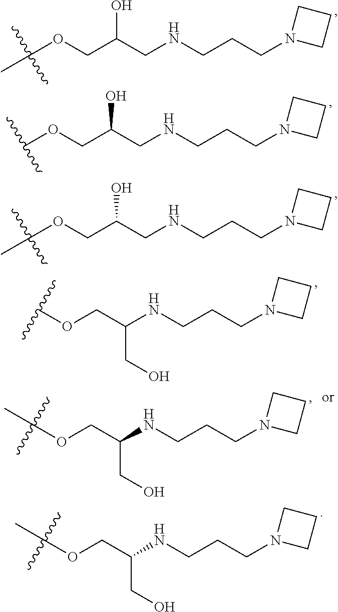

In some embodiments, R^{7c} is

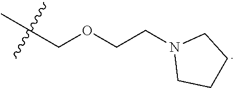 [SMILES: *COCCN1CCCC1]

In some embodiments, R^{7c} is is

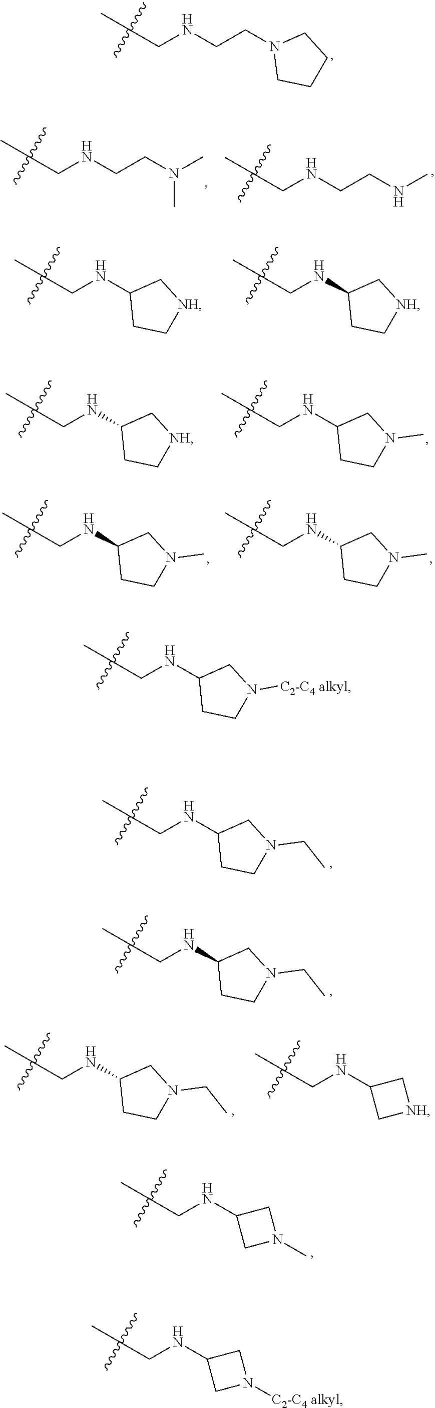

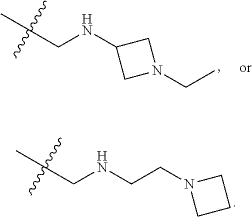

In some embodiments, at least one of R^{8c} and R^{9c} is H. In some embodiments, each of R^{8c} and R is H. In some embodiments, R^{8c} is H.

In some embodiments, R^{9c} is -Q^{4c}-T^{4c}, in which Q^{4c} is a bond or C₁-C₆ alkylene linker optionally substituted with one or more of halo, cyano, hydroxyl, or C₁-C₆ alkoxyl, and T^{4c} is H, halo, OR^{hc}, NR^{hc}R^{ic}, NR^{hc}C(O)R^{ic}, C(O)NR^{hc}R^{ic}, C(O)R^{hc}, C(O)OR^{hc}, or R^{S2C}, in which R^{S2c} is C₃-C₈ cycloalkyl or 4- to 7-membered heterocycloalkyl, and R^{S2c} is optionally substituted with one or more -Q^{5c}-T^{5c}.

In some embodiments, each Q^{5c} independently is a bond or C₁-C₃ alkylene linker.

In some embodiments, each T^{5c} independently is selected from the group consisting of H, halo, cyano, C₁-C₆ alkyl, OR^{jc}, C(O)R^{jc}, C(O)OR^{jc}, NR^{jc}R^{kc}, C(O)NR^{jc}R^{kc}, and NR^{jc}C(O)R^{kc}.

In some embodiments, R^{9c} is C₁-C₃ alkyl.

In some embodiments, R^{14c} is H, halo, or C₁-C₆ alkyl.

In some aspects, the present disclosure provides a compound of Formula (IA′″) or (IIA′″):

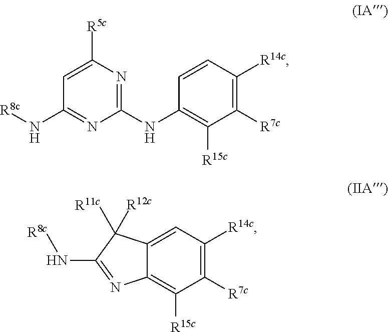

a tautomer thereof, a pharmaceutically acceptable salt thereof, or a pharmaceutically acceptable salt of the tautomer, wherein:
R^{8c} is C₁-C₆ alkyl;
R^{5c} is C₁-C₆ alkyl;
R^{11c} and R^{12c} each independently is C₁-C₆ alkyl, or R^{11c} and R^{12c} together with the carbon atom to which they are attached form C₃-C₁₂ cycloalkyl;
R^{14c} and R^{15c} each independently is H, halogen, or C₁-C₆ alkoxyl; and
R^{7c} is 5- to 10-membered heteroaryl or 4- to 12-membered heterocycloalkyl containing 1-4 heteroatoms selected from N, O, and S, wherein the 5- to 10-membered heteroaryl or 4- to 12-membered heterocycloalkyl is optionally substituted with one or more of R^{7cS}; each R^{7cS} independently is COOH, oxo, C₁-C₆ alkyl, C₁-C₆ haloalkyl, or 4- to 12-membered heterocycloalkyl, wherein the C₁-C₆ alkyl or 4- to 12-membered heterocycloalkyl is optionally substituted with one or more of oxo, C₁-C₆ alkyl, or NR^{7cSa}R^{7cSb}; R^{7cSa} and R^{7cSb} each independently is H or C₁-C₆ alkyl, or R^{7cSa} and R^{7cSb} together with the nitrogen atom to which they are attached form C₃-C₆ heterocycloalkyl.

In some embodiments, the compound is of Formula (IA′″) or (IIA′″), a tautomer thereof, a pharmaceutically acceptable salt thereof, or a pharmaceutically acceptable salt of the tautomer, wherein:
R^{8c} is C₁-C₆ alkyl;
R^{5c} is C₁-C₆ alkyl;
R^{11c} and R^{12c} each independently is C₁-C₆ alkyl, or R^{11c} and R^{12c} together with the carbon atom to which they are attached form C₃-C₁₂ cycloalkyl;
R^{14c} and R^{15c} each independently is H, halogen, or C₁-C₆ alkoxyl; and
R^{7c} is 5- to 10-membered heteroaryl or 4- to 12-membered heterocycloalkyl containing 1-4 heteroatoms selected from N, O, and S, wherein the 5- to 10-membered heteroaryl or 4- to 12-membered heterocycloalkyl is optionally substituted with one or more of R^{7cS}; each R^{7cS} independently is C₁-C₆ alkyl or 4- to 12-membered heterocycloalkyl, wherein the C₁-C₆ alkyl or 4- to 12-membered heterocycloalkyl is optionally substituted with one or more of NR^{7cSa}R^{7cSb}; R^{7cSa} and R^{7cSb} each independently is H or C₁-C₆ alkyl, or R^{7cSa} and R^{7cSb} together with the nitrogen atom to which they are attached form C₃-C₆ heterocycloalkyl.

In some embodiments, R^{8c} is methyl or ethyl. In some embodiments, R^{8c} is methyl.

In some embodiments, R^{5c} is methyl, ethyl, n-propyl, or i-propyl. In some embodiments, R^{5c} is methyl. In some embodiments, R^{5c} is i-propyl.

In some embodiments, R^{11c} and R^{12c} each independently is C₁-C₆ alkyl. In some embodiments, R^{11c} and R^{12c} each independently is methyl, ethyl, n-propyl, i-propyl, n-butyl, i-butyl, s-butyl, t-butyl, pentyl, or hexyl. In some embodiments, R^{11c} and R^{12c} each independently is methyl, ethyl, n-propyl, or i-propyl.

In some embodiments, R^{11c} and R^{12c} together with the carbon atom to which they are attached form C₃-C₁₂ cycloalkyl. In some embodiments, R^{11c} and R^{12c} together with the carbon atom to which they are attached form cyclopropyl, cyclobutyl, cyclopentyl, or cyclohexyl. In some embodiments, R^{11c} and R^{12c} together with the carbon atom to which they are attached form cyclobutyl.

In some embodiments, at least one of R^{11c} and R^{12c} is halogen. In some embodiments, at least one of R^{14c} and R^{15c} is F or Cl. In some embodiments, at least one of R^{14c} and R^{15c} is F. In some embodiments, at least one of R^{14c} and R^{15c} is Cl.

In some embodiments, R^{14c} is halogen. In some embodiments, R^{14c} is F or Cl. In some embodiments, R^{14c} is F. In some embodiments, R^{3c} is Cl.

In some embodiments, R^{15c} is halogen. In some embodiments, R^{15c} is F or Cl. In some embodiments, R^{15c} is F. In some embodiments, R^{15c} is Cl.

In some embodiments, one of R^{14c} and R^{15c} is halogen, and the other one is H or or C₁-C₆ alkoxyl. In some embodiments, at least one of R^{14c} and R^{15c} is F or Cl, and the other one is H or or C₁-C₆ alkoxyl. In some embodiments, at least one of R^{14c} and R^{15c} is F or Cl, and the other one is H. In some embodiments, at least one of R^{14c} and R^{15c} is F or Cl, and the other one is methoxy.

In some embodiments, R^{14c} is halogen, and R^{15c} is H or or C₁-C₆ alkoxyl. In some embodiments, R^{14c} is F or Cl, and R^{15c} is H or or C₁-C₆ alkoxyl. In some embodiments, R^{14c} is F or Cl, and R^{15c} is H. In some embodiments, R^{14c} is F or Cl, and R^{15c} is methoxy.

In some embodiments, R^{15c} is halogen, and R^{14c} is H or or C₁-C₆ alkoxyl. In some embodiments, R^{15c} is F or Cl, and R^{14c} is H or or C₁-C₆ alkoxyl. In some embodiments, R^{15c} is F or Cl, and R^{14c} is H. In some embodiments, R^{15c} is F or Cl, and R^{14c} is methoxy.

In some embodiments, both R^{14c} and R^{15c} are halogen. In some embodiments, R^{14c} and R^{15c} each independently is F or Cl. In some embodiments, both R^{14c} and R^{15c} are F. In some embodiments, R^{14c} is F, and R^{15c} is Cl. In some embodiments, R^{15c} is F, and R^{14c} is Cl. In some embodiments, both R^{14c} and R^{15c} are Cl.

In some embodiments, R^{7c} is 5- to 10-membered heteroaryl containing 1-4 heteroatoms selected from N, O, and S, wherein the 5- to 10-membered heteroaryl is optionally substituted with one or more of R^{7cS}.

In some embodiments, R^{7c} is 5-membered heteroaryl containing 3 of N, wherein the 5-membered heteroaryl is optionally substituted with one or more of R^{7cS}.

In some embodiments, R^{7c} is

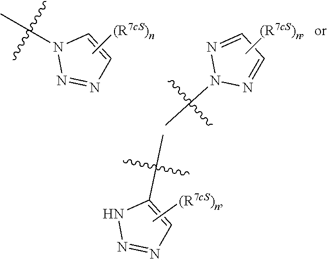

wherein n is 0, 1, or 2.

In some embodiments, R^{7c} is

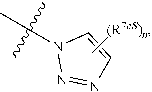

wherein n is 0, 1, or 2.

In some embodiments, the compound is of Formula (IAa′″) or (IIAa′″):

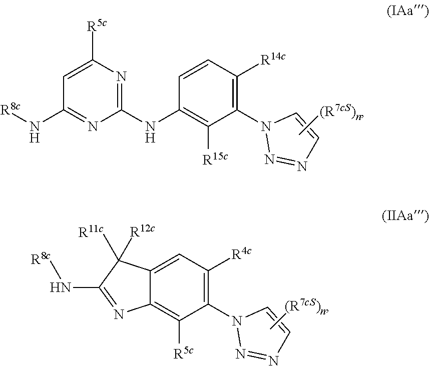

a tautomer thereof, a pharmaceutically acceptable salt thereof, or a pharmaceutically acceptable salt of the tautomer.

In some embodiments, the compound is of Formula (IAb′″) or (IIAb)′″:

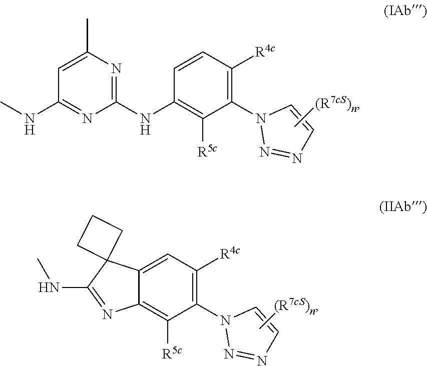

a tautomer thereof, a pharmaceutically acceptable salt thereof, or a pharmaceutically acceptable salt of the tautomer.

In some embodiments, n is 0 or 1. In some embodiments, n is 0. In some embodiments, n is 1.

In some embodiments, R^{7c} is 4- to 12-membered heterocycloalkyl containing 1-4 heteroatoms selected from N, O, and S, wherein the 4- to 12-membered heterocycloalkyl is optionally substituted with one or more of R^{7cS}.

In some embodiments, at least one R^{7cS} is COOH.

In some embodiments, at least one R^{7cS} is oxo.

In some embodiments, at least one R^{7cS} is C₁-C₆ haloalkyl (e.g., methyl, ethyl, propyl, butyl, pental, or hexyl in which at least one H is substituted with a halogen (e.g., F, Cl, Br, or I)). In some embodiments, at least one R^{7cS} is CH₂F, CHF₂, or CF₃. In some embodiments, at least one R^{7cS} is CF₃.

In some embodiments, at least one R^{7cS} is C₁-C₆ alkyl optionally substituted with one or more of oxo or NR^{7cSa}R^{7cSb}. In some embodiments, at least one R^{7cS} is C₁-C₆ alkyl substituted with one oxo and one NR^{7cSa}R^{7cSb}.

In some embodiments, at least one R^{7cS} is C₁-C₆ alkyl optionally substituted with one or more of NR^{7cSa}R^{7cSb} In some embodiments, at least one R^{7cS} is methyl optionally substituted with one or more of NR^{7cSa}R^{7cSb}. In some embodiments, at least one R^{7cS} is

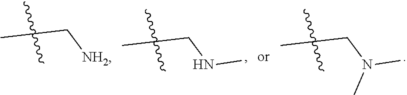

In some embodiments, at least one R^{7cS} is

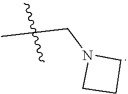 [SMILES: *CN1CCC1]

In some embodiments, at least one R^{7cS} is 4- to 12-membered heterocycloalkyl optionally substituted with one or more of oxo, C₁-C₆ alkyl, or NR^{7cSa}R^{7cSb}. In some embodiments, at least one R^{7cS} is 4- to 12-membered heterocycloalkyl optionally substituted with one or more of C₁-C₆ alkyl.

In some embodiments, at least one R^{7cS} is 4- to 12-membered heterocycloalkyl optionally substituted with one or more of NR^{7cSa}R^{7cSb}. In some embodiments, at least one R^{7cS} is 5-membered heterocycloalkyl optionally substituted with one or more of NR^{7cSa}R^{7cSb}. In some embodiments, at least one R^{7cS} is pyrrolidinyl optionally substituted with one or more of NR^{7cSa}R^{7cSb}. In some embodiments, at least one R^{7cS} is pyrrolidinyl. In some embodiments, at least one R^{7cS} is

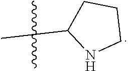 [SMILES: *C1CCCN1]

In some embodiments, at least one R^{7cS} is

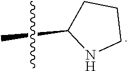

In some embodiments, at least one R^{7cS} is

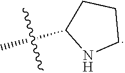

In some embodiments, both of R^{7cSa} and R^{7cSb} are H. In some embodiments, one of R^{7cSa} and R^{7cSb} is H, and the other is C₁-C₆ alkyl. In some embodiments, one of R^{7cSa} and R^{7cSb} is H, and the other is methyl. In some embodiments, both of R^{7cSa} and R^{7cSb} are C₁-C₆ alkyl. In some embodiments, both of R^{7cSa} and R^{7cSb} are methyl.

In some embodiments, R^{7cSa} and R^{7cSb} together with the nitrogen atom to which they are attached form C₃-C₆ heterocycloalkyl. In some embodiments, R^{7cSa} and R^{7cSb} together with the nitrogen atom to which they are attached form C₄ heterocycloalkyl. In some embodiments, R^{7cSa} and R^{7cSb} together with the nitrogen atom to which they are attached form

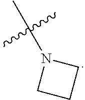 [SMILES: *N1CCC1]

In some embodiments, R^{7c} is

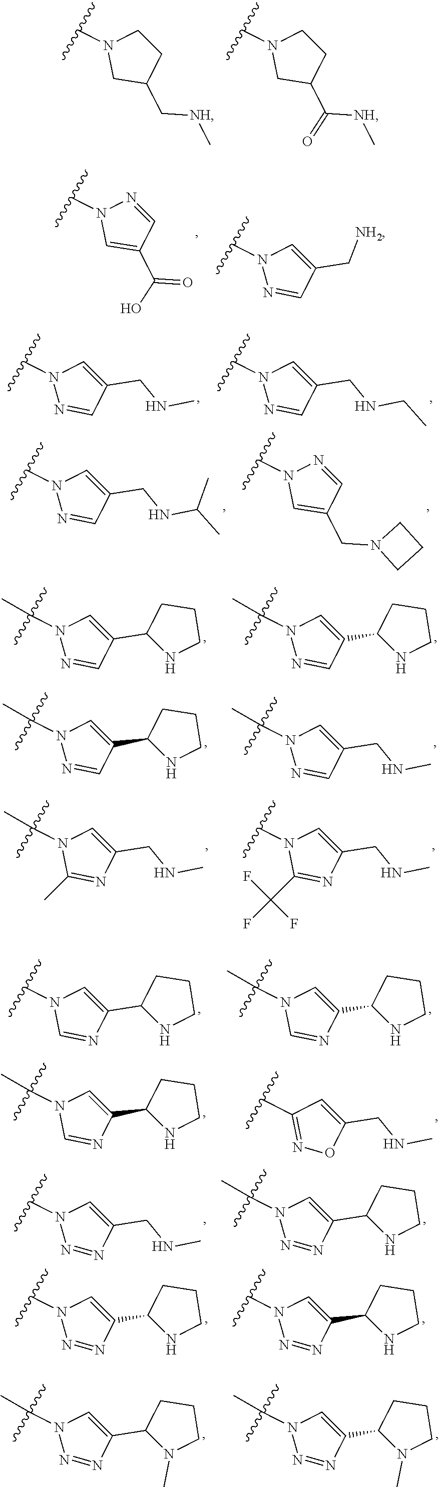

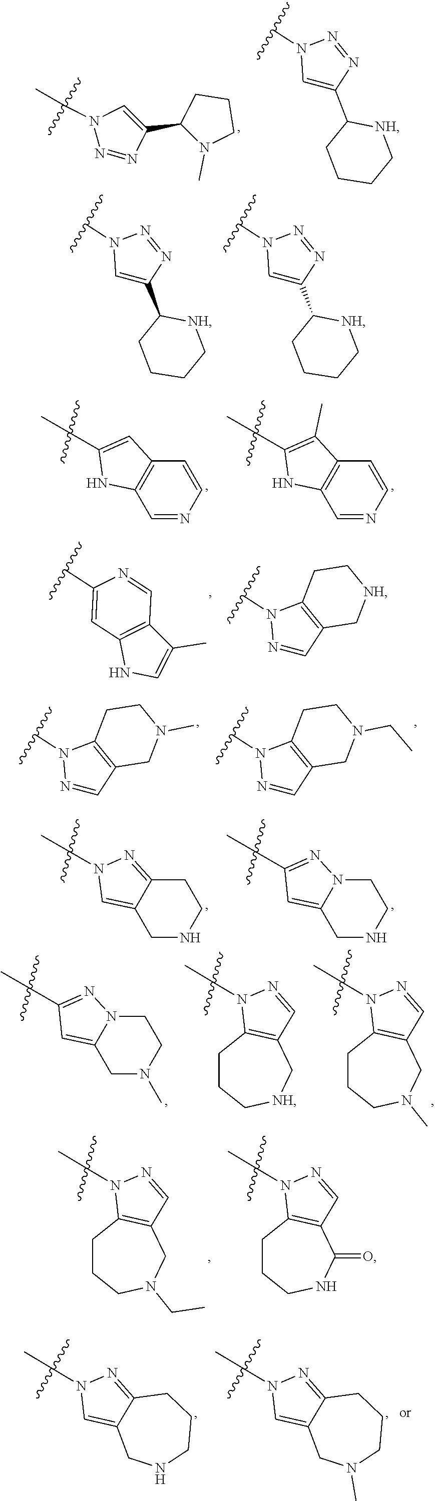

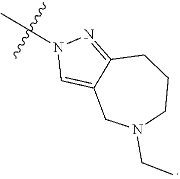 [SMILES: *n1cc2c(n1)CCCN(CC)C2]

In some embodiments, the compound is selected from those in Tables 1A-1E, 2-4, 4A, and 5, tautomers thereof, and pharmaceutically acceptable salts of the compounds and tautomers.

In some embodiments of the methods provided herein, e.g., of the therapeutic methods comprising administering an EHMT2 inhibitor to a subject in need thereof, the EHMT2 inhibitor used is not 2-cyclohexyl-6-methoxy-N-[1-(1-methylethyl)-4-piperidinyl]-7-[3-(1-pyrrolidinyl)propoxy]-4-quinazolinamine; N-(1-isopropylpiperidin-4-yl)-6-methoxy-2-(4-methyl-1,4-diazepan-1-yl)-7-(3-(piperidin-1-yl)propoxy)quinazolin-4-amine; 2-(4,4-difluoropiperidin-1-yl)-N-(1-isopropylpiperidin-4-yl)-6-methoxy-7-(3-(pyrrolidin-1-yl)propoxy)quinazolin-4-amine; or 2-(4-isopropyl-1,4-diazepan-1-yl)-N-(1-isopropylpiperidin-4-yl)-6-methoxy-7-(3-(piperidin-1-yl)propoxy)quinazolin-4-amine.

In some embodiments of the methods provided herein, the EHMT2 inhibitor used is a selective inhibitors of EHMT2.

In some embodiments of the methods provided herein, administration of the EHMT2 inhibitor activates a gene the deactivation of which is associated with a blood disorder. In some embodiments, administration of the EHMT2 inhibitor deactivates a gene the activation of which is associated with a blood disorder.

For example, in some embodiments, administration of the EHMT2 inhibitor activates a gene located on a chromosome selected from the group consisting of 6q24, 7, 11p15.5, 14q32, 15q11q13, 15q11.2, 20q13, and 20. In some embodiments, administration of the EHMT2 inhibitor deactivates a gene located on a chromosome selected from the group consisting of 6q24, 7,11p15.5, 14q32, 15q11q13, 15q11.2, 20q13, and 20.

In some embodiments, administration of the EHMT2 inhibitor inhibits dimethylation of histone 3 at lysine residue 9 (H3K9me2).

In some embodiments, a compound, composition, or treatment modality provided herein, e.g., an EHMT2 inhibitor provided herein, is used in combination with one or more additional therapeutic treatments (e.g., one or more additional therapeutic agent, or one or more intervention), e.g., with one or more approved or experimental treatment of a blood disorder. In some embodiments, the one or more additional therapeutic treatment is an approved or experimental treatment of sickle-cell disease. In some embodiments, the one or more additional therapeutic treatment is an approved or experimental therapeutic agent used for the treatment of sickle-cell disease. For example, in some embodiments, a therapeutic method is provided that comprises administering to a subject having a blood disorder, e.g., sickle-cell disease, an effective amount of an EHMT2 inhibitor provided herein, and one or more therapeutic agent(s) for the treatment of sickle-cell disease. In some embodiments, the method comprises administering to the subject an effective amount of an EHMT2 inhibitor provided herein and an effective amount of hydroxyurea. In some embodiments, the method comprises administering to the subject an effective amount of an EHMT2 inhibitor provided herein and an effective amount of L-glutamine. In some embodiments, the method comprises administering to the subject an effective amount of an EHMT2 inhibitor provided herein, an effective amount of hydroxyurea, and an effective amount of L-glutamine.

In some embodiments, a method of the present disclosure further comprises administering to the subject in need thereof a therapeutically effective amount of one or more additional therapeutic agent. In some embodiments, the EHMT2 inhibitor and the one or more therapeutic agent is administered to the subject in temporal proximity, e.g., at the same time, within an hour, two hours, three hours, four hours, five hours, six hours, eight hours, twelve hours, eighteen hours, one day, two days, three days, four days, five days, six days, one week, two weeks, three weeks, or a month of each other; or, where the administration schedule of the EHMT2 inhibitor and/or the one or more additional therapeutic agent is recurrent over a certain period of time (e.g., recurrent (e.g., daily, twice daily, etc.) doses over several days or weeks), the administration schedule of the EHMT2 inhibitor and of the one or more additional therapeutic agent overlap. In some embodiments, the EHMT2 inhibitor and the one or more additional therapeutic agent is administered simultaneously, sequentially, or alternately.

In some embodiments, a method of the present disclosure comprises administering the EHMT2 inhibitor and the one or more additional therapeutic agent simultaneously. In some embodiments, a method of the present disclosure comprises administering the EHMT2 inhibitor and the one or more additional therapeutic agent sequentially. In some embodiments, a method of the present disclosure further comprises administering the EHMT2 inhibitor and the one or more additional therapeutic agent alternately.

In some embodiments, the EHMT2 inhibitor is administered prior to administering one or more additional therapeutic agent. In some embodiments, one or more additional therapeutic agent is administered prior to administering the EHMT2 inhibitor.

In some embodiments, the one or more additional therapeutic agent comprises a standard-of-care agent, a therapeutic agent for a blood disorder, a histone deacetylase (HDAC) inhibitor, a DNA methyltransferase (DNMT) inhibitor or a hypomethylating agent, a BCL11A inhibitor, a KLF inhibitor, a GATA inhibitor, a c-MYB inhibitor, a PRMT1 inhibitor, a PRMT5 inhibitor, a LSD inhibitor, a P-selectin inhibitor, an immunosuppressive agent, an anti-inflammatory agent, an antihistamine, an aromatic L-amino acid decarboxylase (AADC) or DOPA decarboxylase inhibitor, an immunomodulatory drug, an interleukin-1 beta inhibitor, a cell transplant or a cell population transplant, a clinical intervention associated with preparing a subject for a transplantation procedure, a gene or a protein that induces expression of a target gene or to provide and/or express a functional copy of a gene product in a target cell (e.g., in a blood cell), or any combination thereof.

In some embodiments, the one or more additional therapeutic agent comprises a standard-of-care agent for SCD. In some embodiments, the one or more additional therapeutic agent comprises hydroxyurea. In some embodiments, the one or more additional therapeutic agent comprises L-glutamine. Other standard-of-care agents that can be used in combination with the compounds, compositions, or treatment modalities provided herein are disclosed elsewhere herein or will otherwise be apparent to the person of ordinary skill in the art based on the present disclosure. The disclosure is not limited in this respect.

In some embodiments, the one or more additional therapeutic agent comprises a therapeutic agent for a blood disorder. In some embodiments, the one or more additional therapeutic agent comprises a therapeutic agent for anemia, thalassemia, and/or a hemoglobinopathy, e.g., an agent that increases the number of red blood cells, the amount of functional hemoglobin in the blood, and/or the amount of oxygen-bound hemoglobin in the blood. In some embodiments, the one or more additional therapeutic agent comprises BAX-555 (5-HMF-Aes; 5-hydroxymethyl furfural; Aes-103). In some embodiments, the one or more additional therapeutic agent comprises erythropoietin. In some embodiments, the one or more additional therapeutic agent comprises epogen. In some embodiments, the one or more additional therapeutic agent comprises aranesp. In some embodiments, the one or more additional therapeutic agent comprises Procrit. In some embodiments, the one or more additional therapeutic agent comprises epoetin alfa. In some embodiments, the one or more additional therapeutic agent comprises IMR-687. In some embodiments, the one or more additional therapeutic agent comprises GBT440. In some embodiments, the one or more additional therapeutic agent comprises GCSF. In some embodiments, the one or more additional therapeutic agent comprises isobutyramide. In some embodiments, the one or more additional therapeutic agent comprises anticoagulant treatment. In some embodiments, the anticoagulant treatment comprises a heparin treatment, e.g., tinzaparin.

In some embodiments, the one or more additional therapeutic agent comprises a histone deacetylase (HDAC) inhibitor. In some embodiments, the one or more additional therapeutic agent comprises an HDAC1 inhibitor. In some embodiments, the one or more additional therapeutic agent comprises an HDAC2 inhibitor. In some embodiments, the one or more additional therapeutic agent comprises an HDAC3 inhibitor. In some embodiments, the one or more additional therapeutic agent comprises an HDAC1/2 inhibitor. In some embodiments, the one or more additional therapeutic agent comprises an HDAC1/3 inhibitor. In some embodiments, the one or more additional therapeutic agent comprises an HDAC2/3 inhibitor. In some embodiments, the one or more additional therapeutic agent comprises entinostat. In some embodiments, the one or more additional therapeutic agent comprises vorinostat. In some embodiments, the one or more additional therapeutic agent comprises BG-45.

In some embodiments, the one or more additional therapeutic agent comprises a chemotherapeutic (such as 2CdA, 5-FU, 6-Mercaptopurine, 6-TG, Abraxane™, Accutane®, Actinomycin-D, Adriamycin®, Alimta®, all-trans retinoic acid, amethopterin, Ara-C, Azacitadine, BCNU, Blenoxane®, Camptosar®, CeeNU®, Clofarabine, Clolar™, Cytoxan®, daunorubicin hydrochloride, DaunoXome®, Dacogen®, DIC, Doxil®, Ellence®, Eloxatin®, Emcyt®, etoposide phosphate, Fludara®, FUDR®, Gemzar®, Gleevec®, hexamethylmelamine, Hycamtin®, Hydrea®, Idamycin®, Ifex®, ixabepilone, Ixempra®, L-asparaginase, Leukeran®, liposomal Ara-C, L-PAM, Lysodren, Matulane®, mithracin, Mitomycin-C, Myleran®, Navelbine®, Neutrexin®, nilotinib, Nipent®, Nitrogen Mustard, Novantrone®, Oncaspar®, Panretin®, Paraplatin®, Platinol®, prolifeprospan 20 with carmustine implant, Sandostatin®, Targretin®, Tasigna®, Taxotere®, Temodar®, TESPA, Trisenox®, Valstar®, Velban®, Vidaza™, vincristine sulfate, VM 26, Xeloda® and Zanosar®); biologics (such as Alpha Interferon, Bacillus Calmette-Guerin, Bexxar®, Campath®, Ergamisol®, Erlotinib, Herceptin®, Interleukin-2, Iressa®, lenalidomide, Mylotarg®, Ontak®, Pegasys®, Revlimid®, Rituxan®, Tarceva™, Thalomid®, Velcade® and Zevalin™); a small molecule (such as Tykerb®); a corticosteroid (such as dexamethasone sodium phosphate, DeltaSone® and Delta-Cortef®); a hormonal therapeutic (such as Arimidex®, Aromasin®, Casodex®, Cytadren®, Eligard®, Eulexin®, Evista®, Faslodex®, Femara®, Halotestin®, Megace®, Nilandron®, Nolvadex®, Plenaxis™ and Zoladex®); or a radiopharmaceutical (such as Iodotope®, Metastron®, Phosphocol® and Samarium SM-153).

In some embodiments, the one or more additional therapeutic agent comprises a DNA methyltransferase (DNMT) inhibitor or a hypomethylating agent. In some embodiments, the one or more additional therapeutic agent comprises azacitidine, cytarabine, daunorubicin, decitabine, tetrahydroridine, or any combination thereof. In some embodiments, the one or more additional therapeutic agent comprises azacitidine. In some embodiments, the one or more additional therapeutic agent comprises decitabine. In some embodiments, the one or more additional therapeutic agent comprises decitabine, tetrahydrouridine, or a combination thereof.

In some embodiments, the one or more additional therapeutic agent comprises a BCL11a inhibitor (e.g., a BCL11a inhibitor described in Blood 121(5):830-839 (2013)). In some embodiments, the one or more additional therapeutic agent comprises a KLF inhibitor (e.g., a KLF inhibitor described in Blood 121(5):830-839 (2013)). In some embodiments, the one or more additional therapeutic agent comprises a GATA1 inhibitor. In some embodiments, the one or more additional therapeutic agent comprises a c-MYB inhibitor. In some embodiments, the one or more additional therapeutic agent comprises a PRMT1 inhibitor. In some embodiments, the one or more additional therapeutic agent comprises a PRMT5 inhibitor. In some embodiments, the PRMT1 inhibitors and/or the PRMT5 inhibitor is a PRMT1 inhibitor or a PRMT5 inhibitor described in PCT Application PCT/US2013/77151, filed Dec. 20, 2013; PCT Application PCT/US2013/77221, filed Dec. 20, 2013; PCT Application PCT/US2013/77235, filed Dec. 20, 2013; PCT Application PCT/US2013/77250, filed Dec. 20, 2013; PCT Application PCT/US2013/077308, filed Dec. 20, 2013; PCT Application PCT/US2013/77256, filed Dec. 20, 2013; PCT Application PCT/US2015/037759, filed Jun. 25, 2015; PCT Application PCT/US2015/037768, filed Jun. 25, 2015; PCT Application PCT/US2015/043679, filed Aug. 4, 2015; PCT Application PCT/US2014/029583, filed Mar. 14, 2014; PCT Application PCT/US2014/029710, filed Mar. 14, 2014; PCT Application PCT/US2014/029062, filed Mar. 14, 2014; PCT Application PCT/US2015/050750, filed Sep. 17, 2015; PCT Application PCT/US2014/029009, filed Mar. 14, 2014; PCT Application PCT/US2014/029160, filed Mar. 14, 2014; PCT Application PCT/US2014/029605, filed Mar. 14, 2014; PCT Application PCT/US2014/029665, filed Mar. 14, 2014; PCT Application PCT/US2014/029750, filed Mar. 14, 2014; PCT Application PCT/US2014/029408, filed Mar. 14, 2014; PCT Application PCT/US2015/050675, filed Sep. 17, 2015; PCT Application PCT/US2015/050629, filed Sep. 17, 2015; and/or PCT Application PCT/US2017/016472, filed Feb. 3, 2017, the entire contents of each of which are incorporated herein by reference.

In some embodiments, the one or more additional therapeutic agent comprises a LSD1 inhibitor. In some embodiments, the one or more additional therapeutic agent comprises a P-selectin inhibitor, e.g., a small-molecule P-selectin antagonist or an anti-P-selectin antibody. In some embodiments, the one or more additional therapeutic agent comprises PS1697. In some embodiments, the one or more additional therapeutic agent comprises SelG1 (Crizanlizumab).

In some embodiments, a protein inhibitor described herein (e.g., the BCL11A inhibitor, KLF inhibitor, GATA inhibitor, c-MYB inhibitor, PRMT1 inhibitor, PRMT5 inhibitor, LSD inhibitor, or P-selectin inhibitor) is a small molecule inhibitor. In some embodiments, a protein inhibitor described herein is a nucleic acid mediating protein-targeted RNA interference. For example, in some embodiments, the BCL11a inhibitor is a nucleic acid mediating BCL11a-targeted RNA interference, e.g., a BLC11a-targeted shRNA or siRNA. In some embodiments, a protein inhibitor described herein is an endonuclease that targets a protein-encoding nucleic acid, and mediates a nuclease activity resulting in abolishment or reduction of the protein expression from the protein-encoding nucleic acid. For example, in some embodiments, the BCL11a inhibitor is an endonuclease that targets a BCL11a-encoding nucleic acid, and mediates a nuclease activity resulting in abolishment or reduction of BCL11a expression from the BCL11a-encoding nucleic acid, e.g., a zinc-finger nuclease, a TALE nuclease, or a CRISPR/Cas nuclease. In some embodiments, the one or more additional therapeutic agent comprises a hematopoietic stem cell, e.g., a bone marrow-derived CD34+ cell transduced with a heterologous nucleic acid, e.g., in the form of a viral vector (e.g., a lentiviral vector) encoding a protein inhibitor. For example, in some embodiments, the one or more additional therapeutic agent comprises a hematopoietic stem cell, e.g., a bone marrow-derived CD34+ cell transduced with a heterologous nucleic acid, e.g., in the form of a viral vector (e.g., a lentiviral vector) encoding a BCL11a inhibitor, e.g., encoding a short-hairpin RNA targeting BCL11a or a CRISPR/Cas nuclease targeting BCL11a.

In some embodiments, the one or more additional therapeutic agent comprises an immunosuppressive agent, e.g., an immunosuppressive agent used or useful in the context of an organ or cell transplantation, or in the context of treatment of anemia, e.g., aplastic anemia. In some embodiments, the one or more additional therapeutic agent comprises anti-thymocyte globulin (ATG), e.g., horse- or rabbit-derived ATG. In some embodiments, the one or more additional therapeutic agent comprises cyclosporine, e.g., cyclosporine A. In some embodiments, the one or more additional therapeutic agent comprises mycophenolate mofetil (MMF). In some embodiments, the one or more additional therapeutic agent comprises cyclosporine A and MMF. In some embodiments, the one or more additional therapeutic agent comprises anti-thymocyte globulin (ATG), e.g., derived from horse or rabbit.

In some embodiments, the one or more additional therapeutic agent comprises an anti-inflammatory agent. In some embodiments, the one or more additional therapeutic agent comprises a nonsteroidal anti-inflammatory drug. In some embodiments, the one or more additional therapeutic agent comprises a corticosteroid, e.g., a glucocorticoid. In some embodiments, the one or more additional therapeutic agent comprises prednisone or prednisolone. In some embodiments, the one or more additional therapeutic agent comprises dexamethasone. In some embodiments, the one or more additional therapeutic agent comprises vepoloxamer.

In some embodiments, the one or more additional therapeutic agent comprises an antihistamine. In some embodiments, the antihistamine is an H1 antihistamine. In some embodiments, the antihistamine is desloratidine.

In some embodiments, the one or more additional therapeutic agent comprises an aromatic L-amino acid decarboxylase (AADC) or DOPA decarboxylase inhibitor. In some embodiments, the one or more additional therapeutic agent comprises Benzerazide.

In some embodiments, the one or more additional therapeutic agent comprises an immunomodulatory drug. In some embodiments, the one or more additional therapeutic agent comprises an LSD1-specific inhibitor. In some embodiments, the one or more additional therapeutic agent comprises INCB59872. In some embodiments, the one or more additional therapeutic agent comprises an immune checkpoint inhibitor.

In some embodiments, the one or more additional therapeutic agent comprises an interleukin-1 beta inhibitor. In some embodiments, the one or more additional therapeutic agent comprises Canakinumab.

In some embodiments, the one or more additional therapeutic agent comprises a cell transplant or a cell population transplant, e.g., a blood cell transplant or cell population transplant, or a bone marrow cell transplant or cell population transplant. In some embodiments, the transplant comprises a blood transplant. In some embodiments, the transplant comprises a bone marrow transplant. In some embodiments, the transplant comprises a transplant of a cell population enriched in hematopoietic stem cells. For example, in some embodiments, the transplant comprises the transplant of a cell population enriched in cells expressing CD34 and/or CD133. In some embodiments, the transplant comprises a transplant of a cell population depleted for T-cells or specific sub-populations of T-cells. For example, in some embodiments, the transplant comprises a transplant of a cell population depleted for CD4 and/or CD8 expressing T-cells. In some embodiments, the transplant comprises a transplant of a cell population that is haploidentical with a cell or cell population in the recipient subject, e.g., a haploidentical bone marrow transplant or a haploidentical stem cell transplant. In some embodiments, the transplant comprises a cord blood transplant. In some embodiments, the transplant comprises a transplant of a cell population obtained from cord blood and enriched for CD34 and/or CD133 expressing cells. In some embodiments, the one or more additional therapeutic agent comprises leukapheresis. In some embodiments, the one or more additional therapeutic agent comprises blood transfusion, e.g., whole blood transfusion or transfusion of blood cell populations enriched and/or depleted in certain blood cell subtypes. In some embodiments, the blood transfucion is in the form of a one-time intervention or in the form of a recurring transfuction schedule.

In some embodiments, the one or more additional therapeutic agent comprises a clinical intervention associated with preparing a subject for a transplantation procedure. In some embodiments, the one or more additional therapeutic agent comprises a preparative regimen ablating certain cell populations within the recipient subject, e.g., a myeloablative preparative regimen. In some embodiments, the one or more additional therapeutic agent comprises radiotherapy, e.g., total-body irradiation.

In some embodiments, the one or more additional therapeutic agent comprises a stem cell transplant, e.g., a peripheral blood stem cell transplant, a bone marrow transplant, or a hematopoietic stem cell transplant. In some embodiments, the one or more additional therapeutic agent comprises a cell or plasma exchange, e.g., an amicus red cell exchange. In some embodiments, the transplant is an allogeneic transplant. In some embodiments, the transplant is an autologous transplant, e.g., a cell or cell population is obtained from a subject, treated or expanded ex vivo, and then re-administered to the same subject. In some embodiments of an autologous transplant, cells that are obtained from the subject are dedifferentiated, e.g., into a stem cell or stem-cell-like state, e.g., into an embryonic stem (ES) cell-like state or a hematopoietic stem cell state, and then differentiated into a cell type of interest, e.g., from an ES cell-like state into a hematopoietic stem cell state, or from a hematopoietic stem cell state into a peripheral blood cell state, and then returned to the donor subject.

In some embodiments, a cell is obtained from a subject and a genetic defect is corrected ex vivo before the cell is returned to the donor subject. In some embodiments, a cell is obtained from a donor subject, and a nucleic acid encoding a gene product missing or lacking in the cell, e.g., a nucleic acid encoding a functional hemoglobin gene product, or a portion thereof, is introduced into the cell before the cell is returned to the donor subject. In some embodiments, the nucleic acid is introduced into the cell by viral infection, e.g., by lentiviral infection. In some embodiments, the one or more additional therapeutic agent comprises a treatment of a cell or cell population, e.g., a hematopoietic stem cell population, obtained from a subject expressing a dysfunctional version of the HBB gene encoding the beta chain of hemoglobin with LentiGlobin BB305, thus delivering a functional version of the HBB gene encoding the beta chain of hemoglobin to the cells, before returning the cells to the donor subject. In some embodiments, the cells obtained from the donor are enriched for hematopoietic stem cells (e.g., based on their expression of CD34 and/or CD133) before the cells are contacted with the nucleic acid, e.g., in the form of infection by a lentiviral vector. In some embodiments, the nucleic acid delivered to the cells encodes an anti-sickling form of hemoglobin, or a hemoglobin chain characteristic for an anti-sickling form of hemoglobin, e.g., a with a lentiviral beta-AS3-FB vector.

In some embodiments, a cell is obtained from a donor subject, and a gene or allele associated with a disease or disorder is repaired, knocked out, or silenced in the cell, e.g., by delivering a targeted endonuclease (e.g., a TALE nuclease, zinc finger nuclease, or a CRISPR/Cas nuclease to the cell), or an RNA interference agent (e.g., an shRNA or an siRNA) to the cell.

In some embodiments, the one or more additional therapeutic agent comprises a gene or a protein that induces expression of a target gene or to provide and/or express a functional copy of a gene product in a target cell, e.g., in a blood cell. In some embodiments, the one or more additional therapeutic agent comprises an agent that increases or prolongs the expression of fetal hemoglobin. In some embodiments, the one or more additional therapeutic agent comprises a gene or a protein encoding a transcription factor or cell signaling protein involved in the regulation of fetal hemoglobin. In some embodiments, the one or more additional therapeutic agent comprises a gene or a protein that induces or increases expression of TR2/TR4 or members of the direct repeat eryhtroid definitive (DRED) complex. In some embodiments, the one or more additional therapeutic agent comprises a gene or a protein that is an epigenetic regulator of the human beta globin locus LCR. In some embodiments, the one or more additional therapeutic agent comprises a synthetic zinc finger transcriptional activator, e.g., zinc finger gg1-VP64. In some embodiments, the synthetic zinc finger transcriptional activator targets a locus of (i.e. binds to the DNA of) a fetal or adult hemoglobin gene. In some embodiments the synthetic zinc finger transcriptional activator targets a locus of a gene that regulates the production of fetal or adult hemoglobin. In some embodiments, the one or more additional therapeutic agent comprises an adoptive cell therapy agent. In some embodiments, a functional copy of a fetal or adult hemoglobin gene is inserted into at least one cell of a patient. In some embodiments, the cells are hematopoietic stem cells. In some embodiments, the cells are autologous. In some embodiments, the cells are allogenic. In some embodiments, the functional copy of a fetal or adult hemoglobin gene is inserted into the at least one cell of a patient with a viral vector. In some embodiments, the viral vector encodes a functional copy of a fetal or adult hemoglobin gene. In some embodiments, the viral vector is a lentiviral vector. In some embodiments, the one or more additional therapeutic agent comprises LentiGlobin BB305. In some embodiments, the viral vector is an adenovirus vector, adeno-associated vector (AAV), or a retroviral vector. In some embodiments, the functional copy of a fetal or adult hemoglobin gene is inserted into the at least one cell of a patient using genome engineering. In some embodiments, the genome engineering comprises homologous recombination. In some embodiments, the genome engineering comprises a Cas9, a TALEN, a zinc finger nuclease, an endonuclease or a combination thereof. In some embodiments, the genome engineering repairs a genetic lesion in a hemoglobin locus of the patient to restore function to that locus. In certain embodiments, the genome engineering introduces a functional copy of a hemoglobin gene at another location in the genome.

In some embodiments, the one or more additional therapeutic agent comprises 6R-BH4 (sapropterin), A-001 (Varespladib sodium), Abatacept, Abrisentan, Acetaminophen, Acetylcholine, Aes-103 (BAX-555, 5-hydroxymethyl-2-furfural (5-HMF)), Albuterol, Alemtuzumab, alpha-lipoic acid, acetyl-L-carnitine, ambrisentan, anti-thymocyte globulin (ATG), Apixaban, Arginine (e.g., arginine butyrate, arginine hydrochloride; continuous or loading), aspirin, atorvastatin, azacitadine, azithromycin, benzerazide, BG-45, BMD, BPX-501 (rivogenlecleucel), AP1903 (rimiducid), budesonide, busulfan, busulfex, butyrate, canakinumab, clotrimazole, codeine, cogmed, crizanlizumab, cyclophosphamide (CTX), cyclosporine, dalteparin, decitabine, tetrahydrouridine, deferasirox (ICL670), deferiprone, deferoxamine (DFO), defibrotide, desloratidine, desmopressin, dihydroartemisinin-piperaquine (DP), diphenhydramine, a DNMT inhibitor, docosahexaenoic acid, erythropoietin, hydroxyurea, etinostat, FBS0701, fentanyl citrate, ferriprox, fludarabine, gabapentin, GBT440, GCSF, gene therapy, GMI-1070, granulocyte colony-stimulating factor, GSK1024850A (Synflorix), graft-versus-host-disease (GVHD) prophylaxis, a HDAC inhibitor, a HDAC1/2 inhibitor, HIDA, high dose ICA-17043, HQK-1001, hydromorphone, hydroxyurea, a hypomethylating agent, ICL670, ilaris, intravenous immune globulin, IMR-687, a vaccine (e.g., inactivated influenza A (HIN1) virus vaccine), INCB059872, citrulline, magnesium sulfate, isobutyramide, ketamine, LDV/SOF, LentiGlobin BB305, levetiracetam, L-Glutamine, lidocaine, L-NMMA, losartan, low dose ICA-17043, low dose ketamine, an LSD1 inhibitor, macitentan, magnesium pidolate, a TR2/TR4 agonist, a DRED (direct repeat eryhtroid definitive) agonist, a BCL11 inhibitor, a c-MYB inhibitor, a GATA1 inhibitor, a KLF inhibitor, mefloquine, artesunate, melphalan, memantine hydrochloride, meperidine, mesna (e.g., mesnex), metformin, methadone, methotrexate, methylphenidate, methylprednisolone, prednisone, mometasone furoate, montelukast (e.g., in combination with hydroxyurea), morphine, MP4CO, MST-188 (vepoloxamer), mycophenolate mofetil (MMF), N-acetylcysteine (NAC), niacin-ER, NiCord (ex vivo expanded cell graft derived from umbilical cord stem cells), nitric oxide (e.g., by inhalation), nitroglycerin, NKTT120 (NKT Therapeutics), NO-CO (e.g., by inhalation and expiration), nubain (nalbuphine hydrochloride), NVX-508, omega-3 fatty acids, tetrahydrouridine, L-citrulline, oxypurinol, paludrine, folic acid, panobinostat, PDE9i, penicillin, pentostatin, plerixafor, poloxamer 188, pomalidomide, prasugrel, a PRMT1 inhibitor, a PRMT5 inhibitor, proguanil, propranolol, PSI697, a RAS Inhibitors, r-ATG, recombinant-methionyl human stem cell factor, riociguat, rivaroxaban, rivipansel, sangstat, sanguinate, SC411, SCD-101, SCD-Omegatex, SelG1 (crizanlizumab), sevuparin, siklos (hydroxycarbamide), sildenafil, simvastatin, sirolimus, sodium bicarbonate, sodium nitrite, SPD602 (FBS0701, SSP-004184), sulfadoxine pyrimethamine, synthetic zinc finger transcriptional activators, tacrolimus, t-butylhydroquinone, tDCS plus PES, thiotepa, thymoglobulin, ticagrelor, TLI, treosulfan, tritanrix-HepB/Hib, unfractionated heparin, Vaccination (e.g., Polio Sabin, Prevenar, Pneumo 23), vepoloxamer, vitamin D3, vorinostat, or zileuton, or any combination thereof.

In some embodiments, the one or more additional therapeutic agent comprises hydroxyurea. In some embodiments, the one or more additional therapeutic agent comprises L-Glutamine. In some embodiments, the one or more additional therapeutic agent comprises hydroxyurea and L-Glutamine. Additional, non-limiting examples of some embodiments include those, where the one or more additional therapeutic agent comprises alpha-lipoic acid and acetyl-L-carnitine; BPX-501 and AP1903; cyclosporine A and MMF; decitabine and tetrahydrouridine; erythropoietin and hydroxyurea; mefloquine and artesunate; methylprednisolone and prednisone (e.g., in the form of a prednisone taper); montelukast and hydroxyurea; decitabine and tetrahydrouridine; paludrine and folic acid; paludrine, folic acid, and jobelyn; simvastatin and t-butylhydroquinone; and sulfadoxine-pyrimethamine and amodiaquine.

In some embodiments, the administration of the EHMT2 inhibitor and the one or more additional therapeutic agent results in a pan-cellular induction of HbF.

In some embodiments, the one or more additional therapeutic agent comprises an HbF inducing agent.

In some embodiments, the HbF inducing agent is not an HbF pan cellular inducing agent.

In some embodiments, the one or more additional therapeutic agent comprises an HbF pan cellular inducing agent.

In some embodiments, the one or more additional therapeutic agent does not comprise an HbF pan cellular inducing agent.

In some embodiments, the one or more additional therapeutic agent comprises hydroxyurea.

In some embodiments, the one or more additional therapeutic agent comprises a Pan-HDAC inhibitor.

In some embodiments, the one or more additional therapeutic agent comprises entinostat, vorinostat, or panobinostat.

In some embodiments, the one or more additional therapeutic agent comprises an HDAC inhibitor.

In some embodiments, the one or more additional therapeutic agent comprises an HDAC 1/2 inhibitor. In some embodiments, the one or more additional therapeutic agent comprises Acethylon ACY-957.

In some embodiments, the one or more additional therapeutic agent comprises an HDAC 3 inhibitor. In some embodiments, the one or more additional therapeutic agent comprises Acethylon BG-45.

In some embodiments, the one or more additional therapeutic agent comprises a DMNT1 inhibitor. In some embodiments, the one or more additional therapeutic agent comprises Decitabine.

In some embodiments, the one or more additional therapeutic agent comprises a Decarboxilase inhibitor. In some embodiments, the one or more additional therapeutic agent comprises Benzerazide.

In some embodiments, the one or more additional therapeutic agent comprises an Immunomodulator. In some embodiments, the one or more additional therapeutic agent comprises Pomalidomide.

In some embodiments, the one or more additional therapeutic agent comprises a FOXO-3 Inducer. In some embodiments, the one or more additional therapeutic agent comprises Metformin.

In some embodiments, the one or more additional therapeutic agent comprises a Phosphodiesterase 9 Inhibitor. In some embodiments, the one or more additional therapeutic agent comprises PDE9.

Exemplary EHMT2 inhibitory compounds suitable for use in the methods of the present disclosure include, without limitation, compounds listed in Tables 1A-1E, 2-4, 4A, and 5, and tautomers and salts thereof.

The compounds of Tables 1A-1E are the compounds found in U.S. Application Nos. 62/323,602, 62/348,837, 62/402,997, and Ser. No. 15/601,888, and PCT Application No. PCT/US2017/027918, the entire contents of which are incorporated herein by reference.

TABLE 1A

| Compound | |
|---|---|
| No. | Structure |
| 1 | 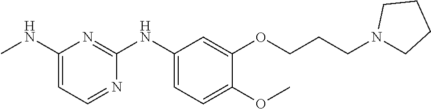 [SMILES: CNc1ccnc(Nc2ccc(OC)c(OCCCN3CCCC3)c2)n1] |
| 2 | 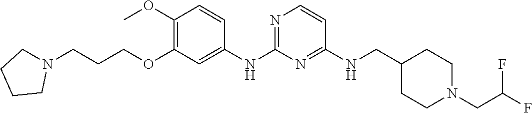 [SMILES: COc1ccc(Nc2nccc(NCC3CCN(CC(F)F)CC3)n2)cc1OCCCN1CCCC1] |
| 3 | 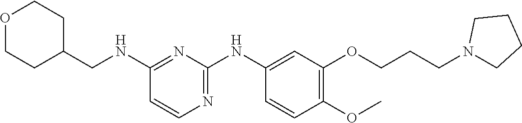 [SMILES: COc1ccc(Nc2nccc(NCC3CCOCC3)n2)cc1OCCCN1CCCC1] |
| 4 | 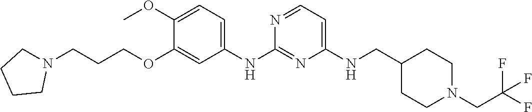 [SMILES: COc1ccc(Nc2nccc(NCC3CCN(CC(F)(F)F)CC3)n2)cc1OCCCN1CCCC1] |
| 5 | 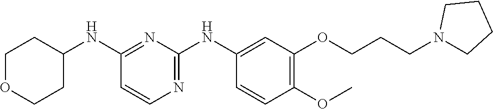 [SMILES: COc1ccc(Nc2nccc(NC3CCOCC3)n2)cc1OCCCN1CCCC1] |
| 6 | 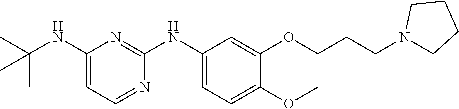 [SMILES: COc1ccc(Nc2nccc(NC(C)(C)C)n2)cc1OCCCN1CCCC1] |
| 7 | 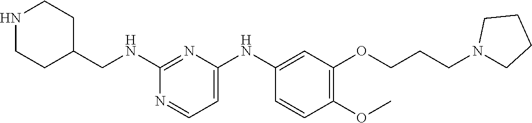 [SMILES: COc1ccc(Nc2ccnc(NCC3CCNCC3)n2)cc1OCCCN1CCCC1] |
| 8 | 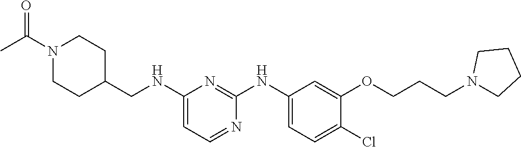 [SMILES: CC(=O)N1CCC(CNc2ccnc(Nc3ccc(Cl)c(OCCCN4CCCC4)c3)n2)CC1] |
| 9 | 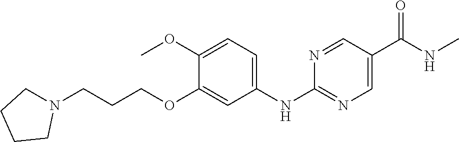 [SMILES: CNC(=O)c1cnc(Nc2ccc(OC)c(OCCCN3CCCC3)c2)nc1] |
| 10 | 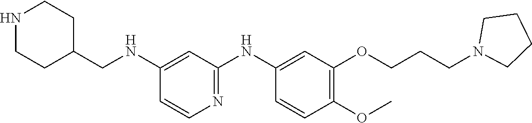 [SMILES: COc1ccc(Nc2cc(NCC3CCNCC3)ccn2)cc1OCCCN1CCCC1] |
| 11 | 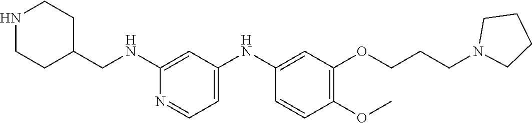 [SMILES: COc1ccc(Nc2ccnc(NCC3CCNCC3)c2)cc1OCCCN1CCCC1] |
| 12 | 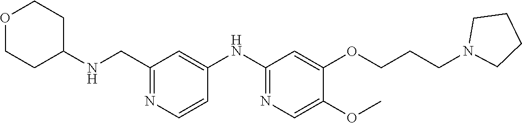 [SMILES: COc1cnc(Nc2ccnc(CNC3CCOCC3)c2)cc1OCCCN1CCCC1] |
| 13 | 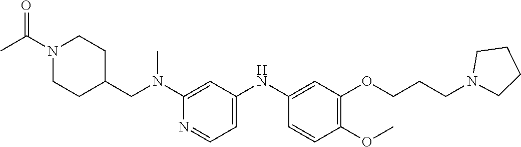 [SMILES: COc1ccc(Nc2ccnc(N(C)CC3CCN(C(C)=O)CC3)c2)cc1OCCCN1CCCC1] |
| 14 | 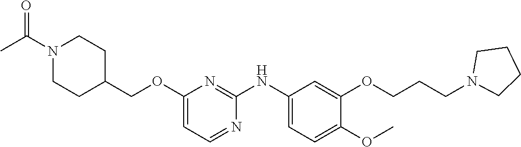 [SMILES: COc1ccc(Nc2nccc(OCC3CCN(C(C)=O)CC3)n2)cc1OCCCN1CCCC1] |
| 15 | 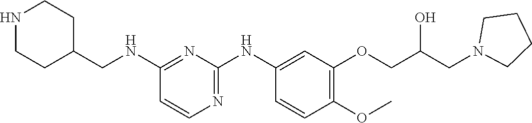 [SMILES: COc1ccc(Nc2nccc(NCC3CCNCC3)n2)cc1OCC(O)CN1CCCC1] |
| 16 | 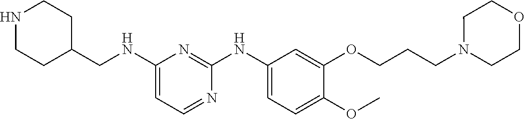 [SMILES: COc1ccc(Nc2nccc(NCC3CCNCC3)n2)cc1OCCCN1CCOCC1] |
| 17 | 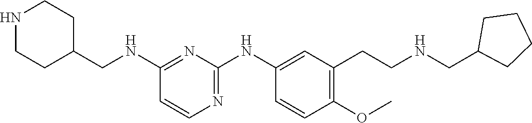 [SMILES: COc1ccc(Nc2nccc(NCC3CCNCC3)n2)cc1CCNCC1CCCC1] |
| 18 | 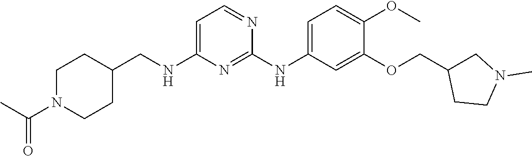 [SMILES: COc1ccc(Nc2nccc(NCC3CCN(C(C)=O)CC3)n2)cc1OCC1CCN(C)C1] |
| 19 | 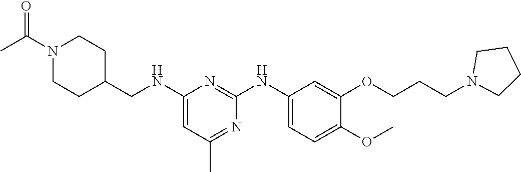 [SMILES: COc1ccc(Nc2nc(C)cc(NCC3CCN(C(C)=O)CC3)n2)cc1OCCCN1CCCC1] |
| 20 | 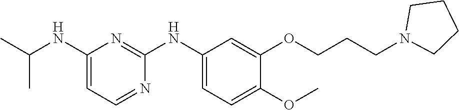 [SMILES: COc1ccc(Nc2nccc(NC(C)C)n2)cc1OCCCN1CCCC1] |
| 21 | 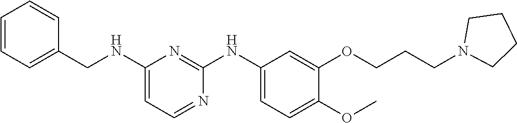 [SMILES: COc1ccc(Nc2nccc(NCc3ccccc3)n2)cc1OCCCN1CCCC1] |
| 22 | 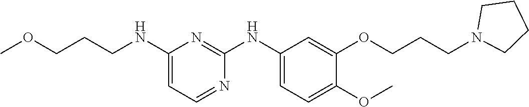 [SMILES: COCCCNc1ccnc(Nc2ccc(OC)c(OCCCN3CCCC3)c2)n1] |
| 23 | 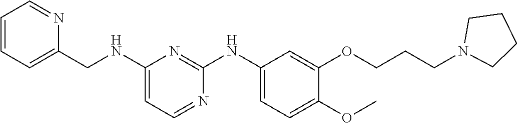 [SMILES: COc1ccc(Nc2nccc(NCc3ccccn3)n2)cc1OCCCN1CCCC1] |
| 24 | 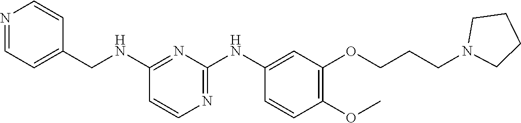 [SMILES: COc1ccc(Nc2nccc(NCc3ccncc3)n2)cc1OCCCN1CCCC1] |
| 25 | 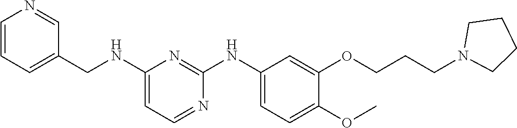 [SMILES: COc1ccc(Nc2nccc(NCc3cccnc3)n2)cc1OCCCN1CCCC1] |
| 26 | 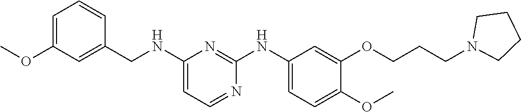 [SMILES: COc1cccc(CNc2ccnc(Nc3ccc(OC)c(OCCCN4CCCC4)c3)n2)c1] |
| 27 | 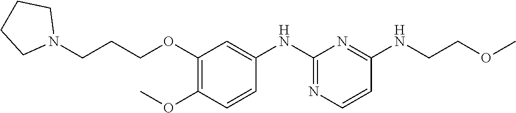 [SMILES: COCCNc1ccnc(Nc2ccc(OC)c(OCCCN3CCCC3)c2)n1] |
| 28 | 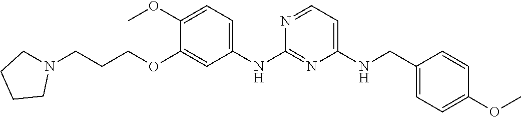 [SMILES: COc1ccc(CNc2ccnc(Nc3ccc(OC)c(OCCCN4CCCC4)c3)n2)cc1] |
| 29 | 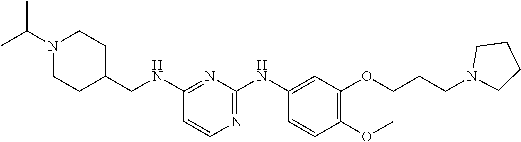 [SMILES: COc1ccc(Nc2nccc(NCC3CCN(C(C)C)CC3)n2)cc1OCCCN1CCCC1] |
| 30 | 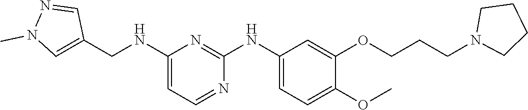 [SMILES: COc1ccc(Nc2nccc(NCc3cnn(C)c3)n2)cc1OCCCN1CCCC1] |
| 31 | 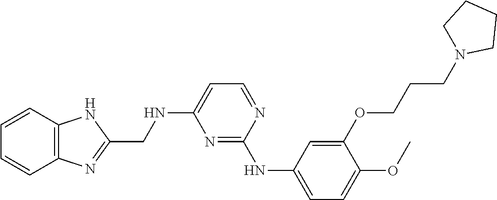 [SMILES: COc1ccc(Nc2nccc(NCc3nc4ccccc4[nH]3)n2)cc1OCCCN1CCCC1] |
| 32 | 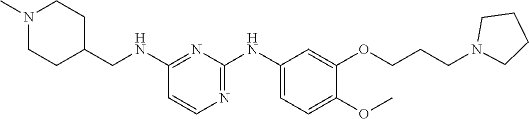 [SMILES: COc1ccc(Nc2nccc(NCC3CCN(C)CC3)n2)cc1OCCCN1CCCC1] |
| 33 | 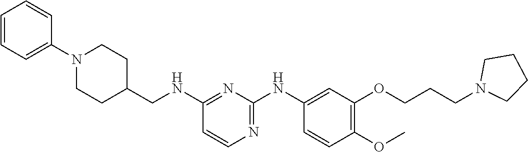 [SMILES: COc1ccc(Nc2nccc(NCC3CCN(c4ccccc4)CC3)n2)cc1OCCCN1CCCC1] |
| 34 | 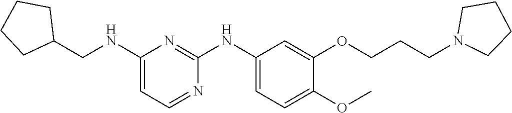 [SMILES: COc1ccc(Nc2nccc(NCC3CCCC3)n2)cc1OCCCN1CCCC1] |
| 35 | 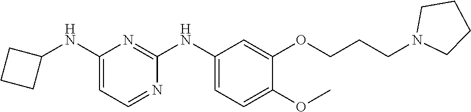 [SMILES: COc1ccc(Nc2nccc(NC3CCC3)n2)cc1OCCCN1CCCC1] |
| 36 | 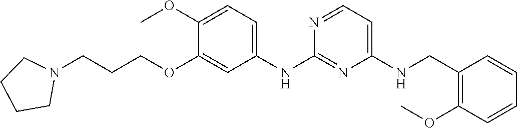 [SMILES: COc1ccccc1CNc1ccnc(Nc2ccc(OC)c(OCCCN3CCCC3)c2)n1] |
| 37 | 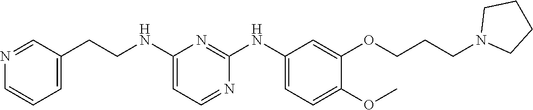 [SMILES: COc1ccc(Nc2nccc(NCCc3cccnc3)n2)cc1OCCCN1CCCC1] |
| 38 | 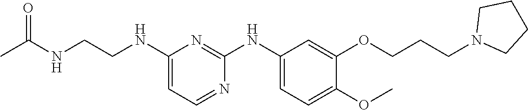 [SMILES: COc1ccc(Nc2nccc(NCCNC(C)=O)n2)cc1OCCCN1CCCC1] |
| 39 | 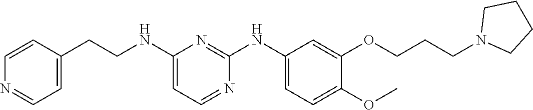 [SMILES: COc1ccc(Nc2nccc(NCCc3ccncc3)n2)cc1OCCCN1CCCC1] |
| 40 | 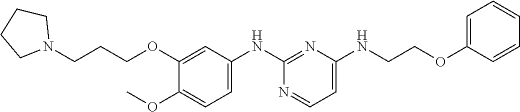 [SMILES: COc1ccc(Nc2nccc(NCCOc3ccccc3)n2)cc1OCCCN1CCCC1] |
| 41 | 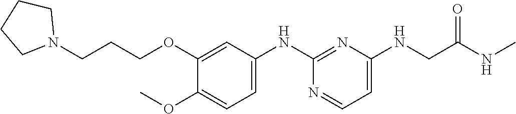 [SMILES: CNC(=O)CNc1ccnc(Nc2ccc(OC)c(OCCCN3CCCC3)c2)n1] |
| 42 | 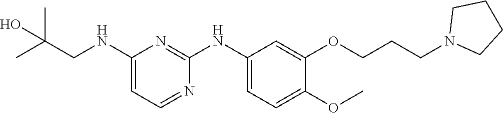 [SMILES: COc1ccc(Nc2nccc(NCC(C)(C)O)n2)cc1OCCCN1CCCC1] |
| 43 | 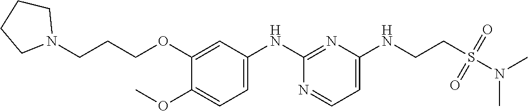 [SMILES: COc1ccc(Nc2nccc(NCCS(=O)(=O)N(C)C)n2)cc1OCCCN1CCCC1] |
| 44 | 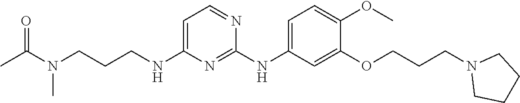 [SMILES: COc1ccc(Nc2nccc(NCCCN(C)C(C)=O)n2)cc1OCCCN1CCCC1] |
| 45 | 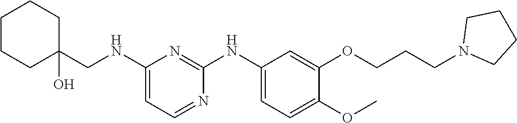 [SMILES: COc1ccc(Nc2nccc(NCC3(O)CCCCC3)n2)cc1OCCCN1CCCC1] |
| 46 | 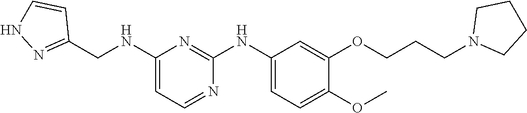 [SMILES: COc1ccc(Nc2nccc(NCc3cc[nH]n3)n2)cc1OCCCN1CCCC1] |
| 47 | 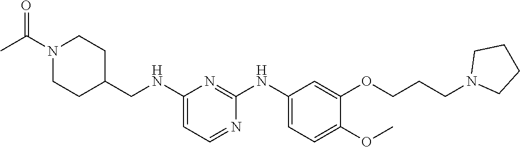 [SMILES: COc1ccc(Nc2nccc(NCC3CCN(C(C)=O)CC3)n2)cc1OCCCN1CCCC1] |
| 48 | 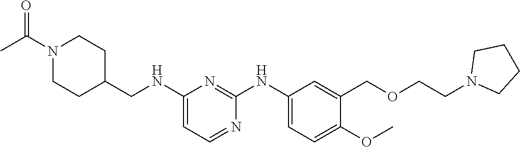 [SMILES: COc1ccc(Nc2nccc(NCC3CCN(C(C)=O)CC3)n2)cc1COCCN1CCCC1] |
| 49 | 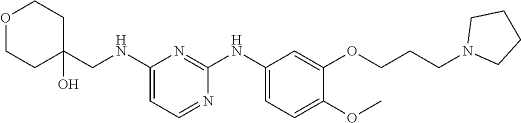 [SMILES: COc1ccc(Nc2nccc(NCC3(O)CCOCC3)n2)cc1OCCCN1CCCC1] |
| 50 | 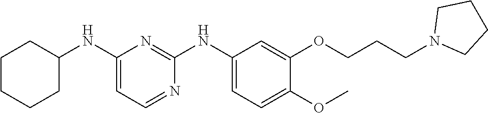 [SMILES: COc1ccc(Nc2nccc(NC3CCCCC3)n2)cc1OCCCN1CCCC1] |
| 51 | 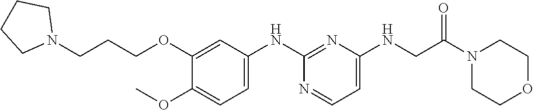 [SMILES: COc1ccc(Nc2nccc(NCC(=O)N3CCOCC3)n2)cc1OCCCN1CCCC1] |
| 52 | 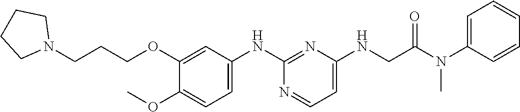 [SMILES: COc1ccc(Nc2nccc(NCC(=O)N(C)c3ccccc3)n2)cc1OCCCN1CCCC1] |
| 53 | 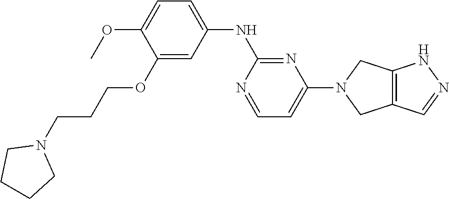 [SMILES: COc1ccc(Nc2nccc(N3Cc4cn[nH]c4C3)n2)cc1OCCCN1CCCC1] |
| 54 | 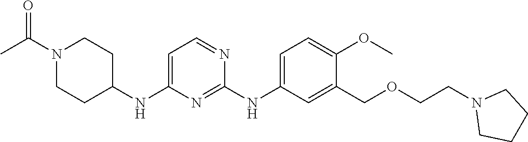 [SMILES: COc1ccc(Nc2nccc(NC3CCN(C(C)=O)CC3)n2)cc1COCCN1CCCC1] |
| 55 | 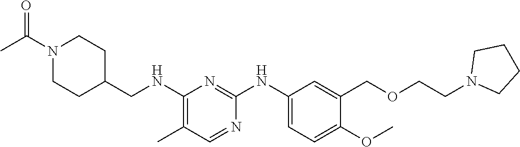 [SMILES: COc1ccc(Nc2ncc(C)c(NCC3CCN(C(C)=O)CC3)n2)cc1COCCN1CCCC1] |
| 56 | 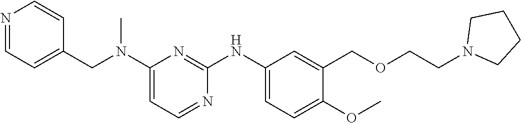 [SMILES: COc1ccc(Nc2nccc(N(C)Cc3ccncc3)n2)cc1COCCN1CCCC1] |
| 57 | 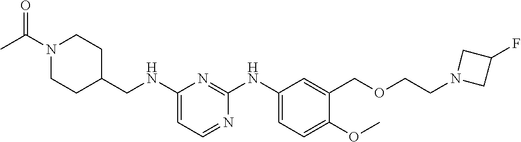 [SMILES: COc1ccc(Nc2nccc(NCC3CCN(C(C)=O)CC3)n2)cc1COCCN1CC(F)C1] |
| 58 | 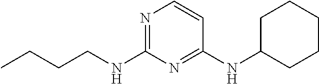 [SMILES: CCCCNc1nccc(NC2CCCCC2)n1] |
| 59 | 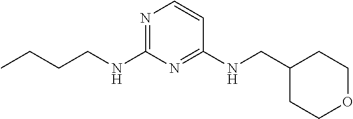 [SMILES: CCCCNc1nccc(NCC2CCOCC2)n1] |
| 60 | 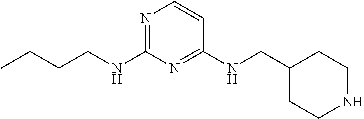 [SMILES: CCCCNc1nccc(NCC2CCNCC2)n1] |
| 61 | 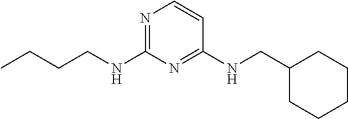 [SMILES: CCCCNc1nccc(NCC2CCCCC2)n1] |
| 62 | 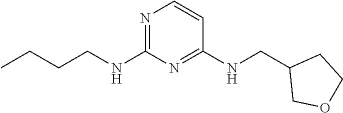 [SMILES: CCCCNc1nccc(NCC2CCOC2)n1] |
| 63 | 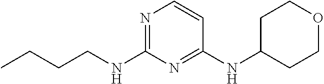 [SMILES: CCCCNc1nccc(NC2CCOCC2)n1] |
| 64 | 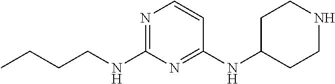 [SMILES: CCCCNc1nccc(NC2CCNCC2)n1] |
| 65 | 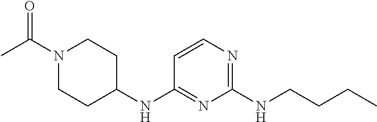 [SMILES: CCCCNc1nccc(NC2CCN(C(C)=O)CC2)n1] |
| 66 | 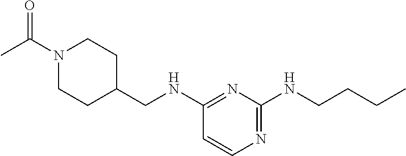 [SMILES: CCCCNc1nccc(NCC2CCN(C(C)=O)CC2)n1] |
| 67 | 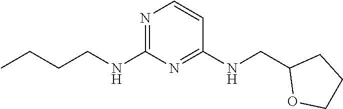 [SMILES: CCCCNc1nccc(NCC2CCCO2)n1] |
| 68 | 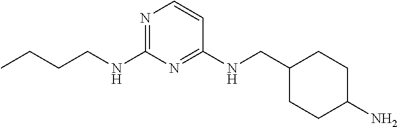 [SMILES: CCCCNc1nccc(NCC2CCC(N)CC2)n1] |
| 69 | 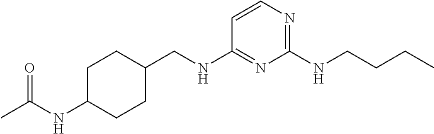 [SMILES: CCCCNc1nccc(NCC2CCC(NC(C)=O)CC2)n1] |
| 70 | 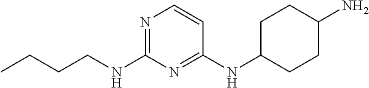 [SMILES: CCCCNc1nccc(NC2CCC(N)CC2)n1] |
| 71 | 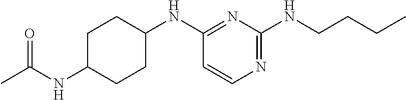 [SMILES: CCCCNc1nccc(NC2CCC(NC(C)=O)CC2)n1] |
| 72 | 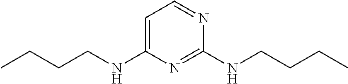 [SMILES: CCCCNc1ccnc(NCCCC)n1] |
| 73 | 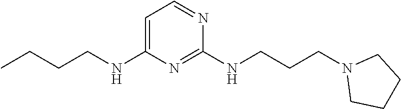 [SMILES: CCCCNc1ccnc(NCCCN2CCCC2)n1] |
| 74 | 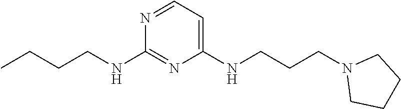 [SMILES: CCCCNc1nccc(NCCCN2CCCC2)n1] |
| 75 | 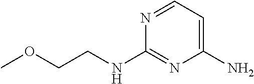 |
| 76 | 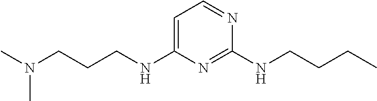 [SMILES: CCCCNc1nccc(NCCCN(C)C)n1] |
| 77 | 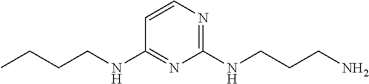 [SMILES: CCCCNc1ccnc(NCCCN)n1] |
| 78 | 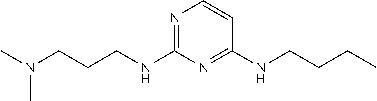 [SMILES: CCCCNc1ccnc(NCCCN(C)C)n1] |
| 79 | 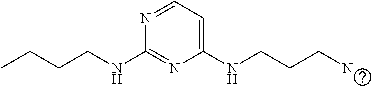 [SMILES: CCCCNc1nccc(NCCCN)n1] |
| 80 | 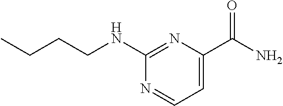 |
| 81 | 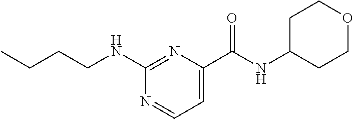 [SMILES: CCCCNc1nccc(C(=O)NC2CCOCC2)n1] |
| 82 | 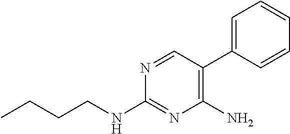 [SMILES: CCCCNc1ncc(-c2ccccc2)c(N)n1] |
| 83 | 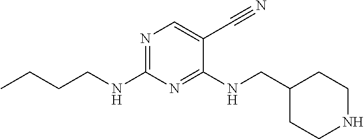 [SMILES: CCCCNc1ncc(C#N)c(NCC2CCNCC2)n1] |
| 84 | 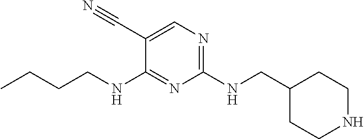 [SMILES: CCCCNc1nc(NCC2CCNCC2)ncc1C#N] |
| 85 | 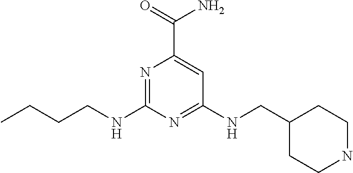 [SMILES: CCCCNc1nc(NCC2CCNCC2)cc(C(N)=O)n1] |
| 86 | 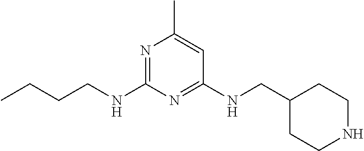 [SMILES: CCCCNc1nc(C)cc(NCC2CCNCC2)n1] |
| 87 | 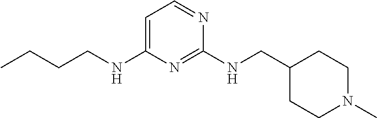 [SMILES: CCCCNc1ccnc(NCC2CCN(C)CC2)n1] |
| 88 | 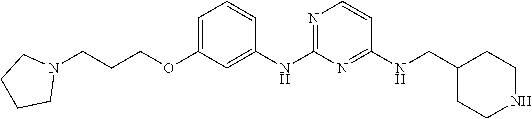 [SMILES: c1cc(Nc2nccc(NCC3CCNCC3)n2)cc(OCCCN2CCCC2)c1] |
| 89 | 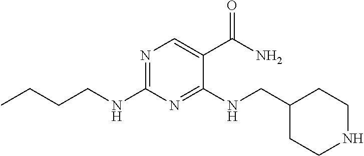 [SMILES: CCCCNc1ncc(C(N)=O)c(NCC2CCNCC2)n1] |
| 90 | 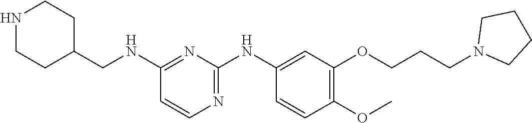 [SMILES: COc1ccc(Nc2nccc(NCC3CCNCC3)n2)cc1OCCCN1CCCC1] |
| 91 | 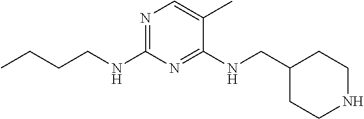 [SMILES: CCCCNc1ncc(C)c(NCC2CCNCC2)n1] |
| 92 | 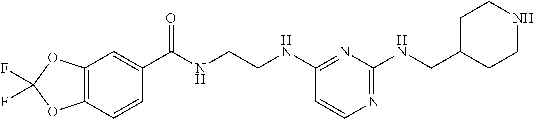 [SMILES: O=C(NCCNc1ccnc(NCC2CCNCC2)n1)c1ccc2c(c1)OC(F)(F)O2] |
| 93 | 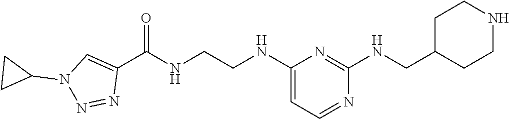 [SMILES: O=C(NCCNc1ccnc(NCC2CCNCC2)n1)c1cn(C2CC2)nn1] |
| 94 | 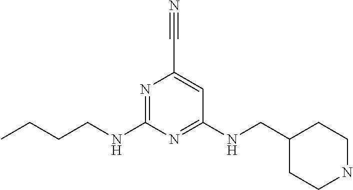 [SMILES: CCCCNc1nc(C#N)cc(NCC2CCNCC2)n1] |
| 95 | 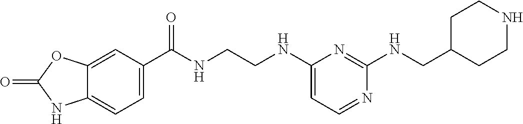 [SMILES: O=C(NCCNc1ccnc(NCC2CCNCC2)n1)c1ccc2[nH]c(=O)oc2c1] |
| 96 | 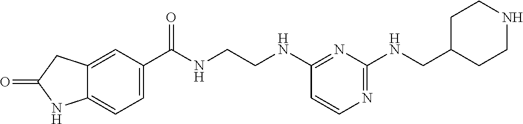 [SMILES: O=C1Cc2cc(C(=O)NCCNc3ccnc(NCC4CCNCC4)n3)ccc2N1] |
| 97 | 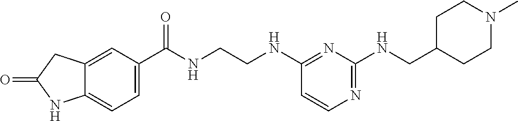 [SMILES: CN1CCC(CNc2nccc(NCCNC(=O)c3ccc4c(c3)CC(=O)N4)n2)CC1] |
| 98 | 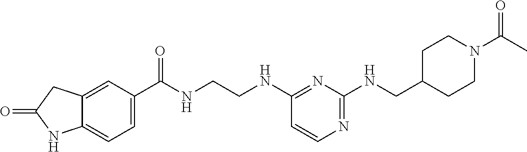 [SMILES: CC(=O)N1CCC(CNc2nccc(NCCNC(=O)c3ccc4c(c3)CC(=O)N4)n2)CC1] |
| 99 | 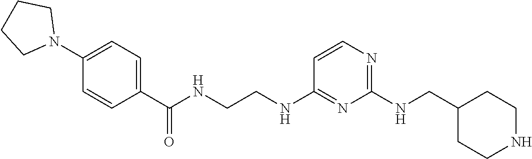 [SMILES: O=C(NCCNc1ccnc(NCC2CCNCC2)n1)c1ccc(N2CCCC2)cc1] |
| 100 | 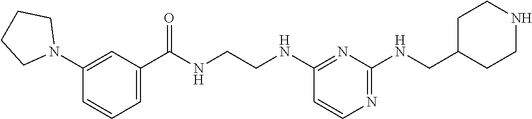 [SMILES: O=C(NCCNc1ccnc(NCC2CCNCC2)n1)c1cccc(N2CCCC2)c1] |
| 101 | 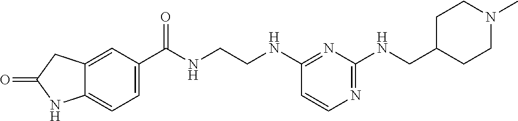 [SMILES: CN1CCC(CNc2nccc(NCCNC(=O)c3ccc4c(c3)CC(=O)N4)n2)CC1] |
| 102 | 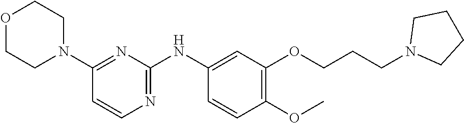 [SMILES: COc1ccc(Nc2nccc(N3CCOCC3)n2)cc1OCCCN1CCCC1] |
| 103 | 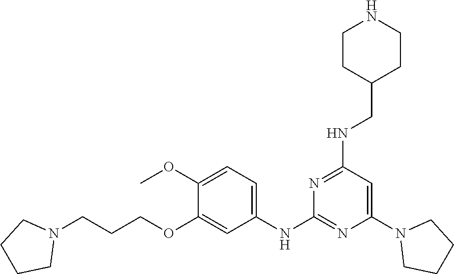 [SMILES: COc1ccc(Nc2nc(NCC3CCNCC3)cc(N3CCCC3)n2)cc1OCCCN1CCCC1] |
| 104 | 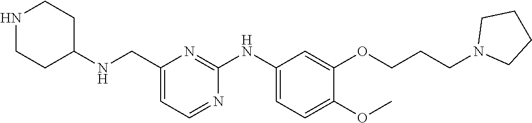 [SMILES: COc1ccc(Nc2nccc(CNC3CCNCC3)n2)cc1OCCCN1CCCC1] |
| 105 | 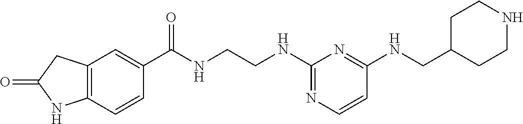 [SMILES: O=C1Cc2cc(C(=O)NCCNc3nccc(NCC4CCNCC4)n3)ccc2N1] |
| 106 | 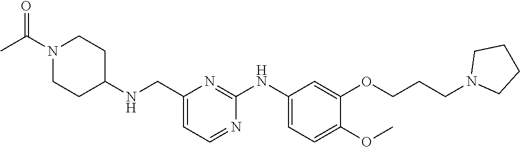 [SMILES: COc1ccc(Nc2nccc(CNC3CCN(C(C)=O)CC3)n2)cc1OCCCN1CCCC1] |
| 107 | 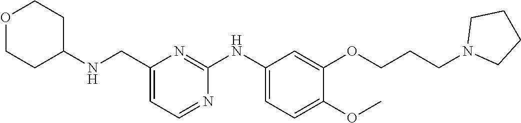 [SMILES: COc1ccc(Nc2nccc(CNC3CCOCC3)n2)cc1OCCCN1CCCC1] |
| 108 | 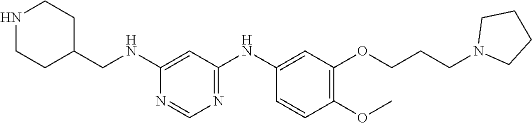 [SMILES: COc1ccc(Nc2cc(NCC3CCNCC3)ncn2)cc1OCCCN1CCCC1] |
| 109 | 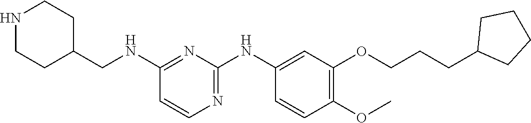 [SMILES: COc1ccc(Nc2nccc(NCC3CCNCC3)n2)cc1OCCCC1CCCC1] |
| 110 | 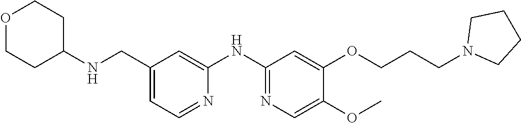 [SMILES: COc1cnc(Nc2cc(CNC3CCOCC3)ccn2)cc1OCCCN1CCCC1] |
| 111 | 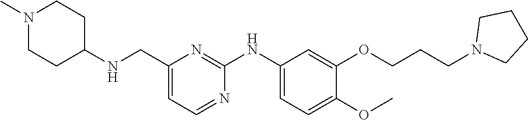 [SMILES: COc1ccc(Nc2nccc(CNC3CCN(C)CC3)n2)cc1OCCCN1CCCC1] |
| 112 | 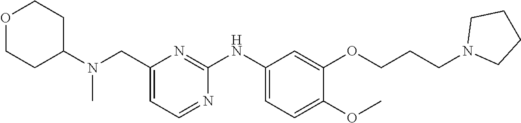 [SMILES: COc1ccc(Nc2nccc(CN(C)C3CCOCC3)n2)cc1OCCCN1CCCC1] |
| 113 | 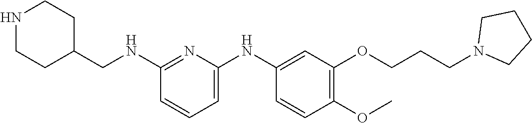 [SMILES: COc1ccc(Nc2cccc(NCC3CCNCC3)n2)cc1OCCCN1CCCC1] |
| 114 | 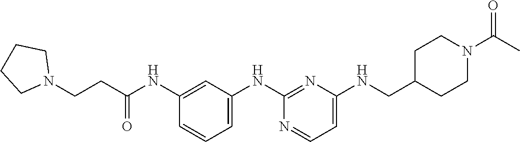 [SMILES: CC(=O)N1CCC(CNc2ccnc(Nc3cccc(NC(=O)CCN4CCCC4)c3)n2)CC1] |
| 115 | 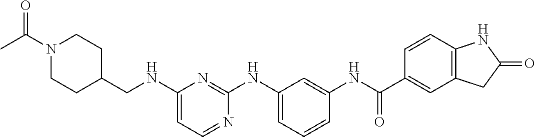 [SMILES: CC(=O)N1CCC(CNc2ccnc(Nc3cccc(NC(=O)c4ccc5c(c4)CC(=O)N5)c3)n2)CC1] |
| 116 | 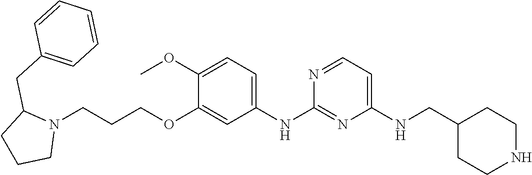 [SMILES: COc1ccc(Nc2nccc(NCC3CCNCC3)n2)cc1OCCCN1CCCC1Cc1ccccc1] |
| 117 | 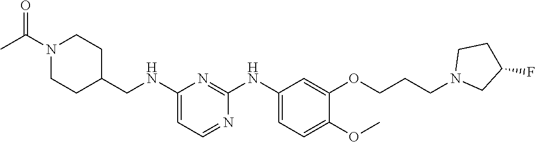 [SMILES: COc1ccc(Nc2nccc(NCC3CCN(C(C)=O)CC3)n2)cc1OCCCN1CC[C@H](F)C1] |
| 118 | 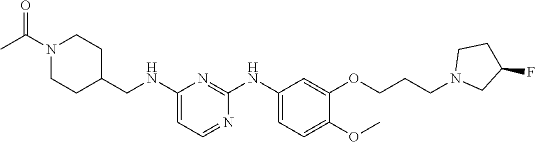 [SMILES: COc1ccc(Nc2nccc(NCC3CCN(C(C)=O)CC3)n2)cc1OCCCN1CC[C@@H](F)C1] |
| 119 | 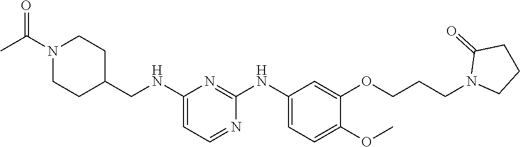 [SMILES: COc1ccc(Nc2nccc(NCC3CCN(C(C)=O)CC3)n2)cc1OCCCN1CCCC1=O] |
| 120 | 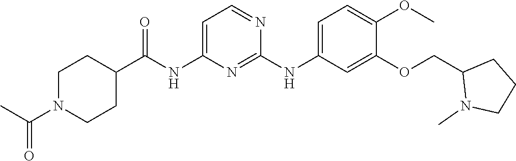 [SMILES: COc1ccc(Nc2nccc(NC(=O)C3CCN(C(C)=O)CC3)n2)cc1OCC1CCCN1C] |
| 121 | 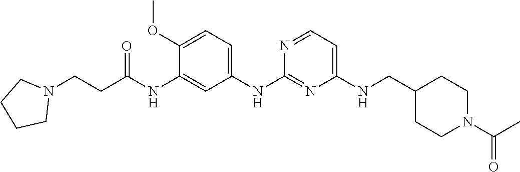 [SMILES: COc1ccc(Nc2nccc(NCC3CCN(C(C)=O)CC3)n2)cc1NC(=O)CCN1CCCC1] |
| 122 | 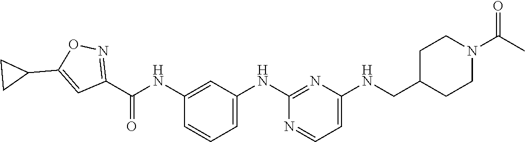 [SMILES: CC(=O)N1CCC(CNc2ccnc(Nc3cccc(NC(=O)c4cc(C5CC5)on4)c3)n2)CC1] |
| 123 | 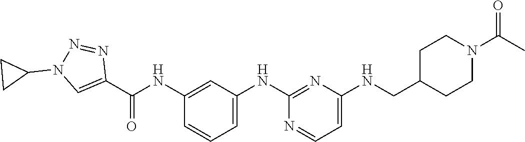 [SMILES: CC(=O)N1CCC(CNc2ccnc(Nc3cccc(NC(=O)c4cn(C5CC5)nn4)c3)n2)CC1] |
| 124 | 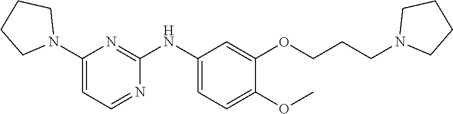 [SMILES: COc1ccc(Nc2nccc(N3CCCC3)n2)cc1OCCCN1CCCC1] |
| 125 | 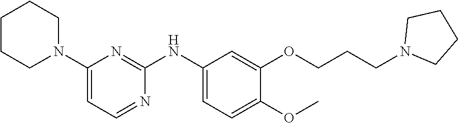 [SMILES: COc1ccc(Nc2nccc(N3CCCCC3)n2)cc1OCCCN1CCCC1] |
| 126 | 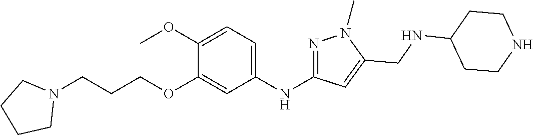 [SMILES: COc1ccc(Nc2cc(CNC3CCNCC3)n(C)n2)cc1OCCCN1CCCC1] |
| 127 | 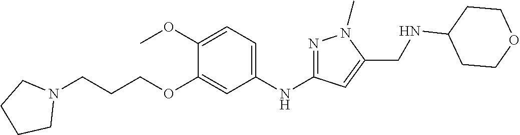 [SMILES: COc1ccc(Nc2cc(CNC3CCOCC3)n(C)n2)cc1OCCCN1CCCC1] |
| 128 | 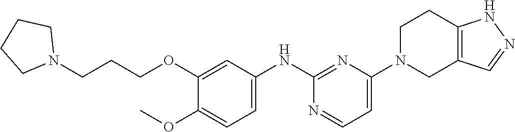 [SMILES: COc1ccc(Nc2nccc(N3CCc4[nH]ncc4C3)n2)cc1OCCCN1CCCC1] |
| 129 | 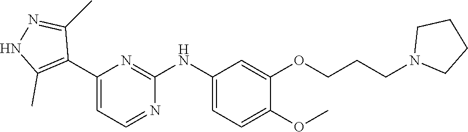 [SMILES: COc1ccc(Nc2nccc(-c3c(C)n[nH]c3C)n2)cc1OCCCN1CCCC1] |
| 130 | 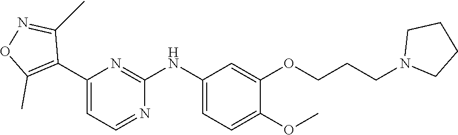 [SMILES: COc1ccc(Nc2nccc(-c3c(C)noc3C)n2)cc1OCCCN1CCCC1] |
| 131 | 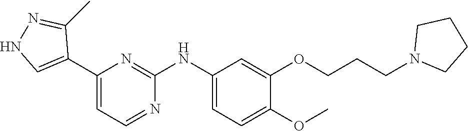 [SMILES: COc1ccc(Nc2nccc(-c3c[nH]nc3C)n2)cc1OCCCN1CCCC1] |
| 132 | 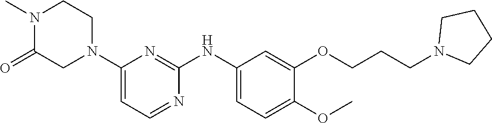 [SMILES: COc1ccc(Nc2nccc(N3CCN(C)C(=O)C3)n2)cc1OCCCN1CCCC1] |
| 133 | 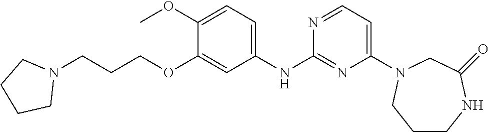 [SMILES: COc1ccc(Nc2nccc(N3CCCNC(=O)C3)n2)cc1OCCCN1CCCC1] |
| 134 | 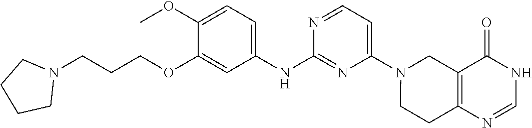 [SMILES: COc1ccc(Nc2nccc(N3CCc4nc[nH]c(=O)c4C3)n2)cc1OCCCN1CCCC1] |
| 135 | 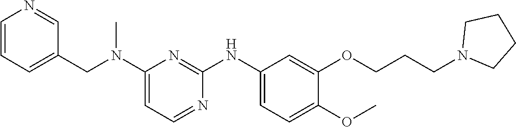 [SMILES: COc1ccc(Nc2nccc(N(C)Cc3cccnc3)n2)cc1OCCCN1CCCC1] |
| 136 | 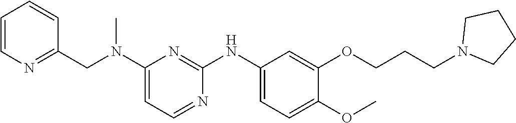 [SMILES: COc1ccc(Nc2nccc(N(C)Cc3ccccn3)n2)cc1OCCCN1CCCC1] |
| 137 | 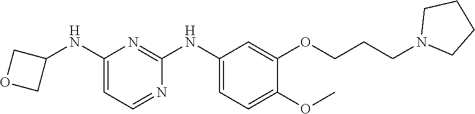 [SMILES: COc1ccc(Nc2nccc(NC3COC3)n2)cc1OCCCN1CCCC1] |
| 138 | 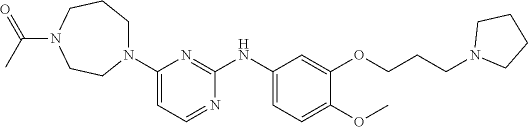 [SMILES: COc1ccc(Nc2nccc(N3CCCN(C(C)=O)CC3)n2)cc1OCCCN1CCCC1] |
| 139 | 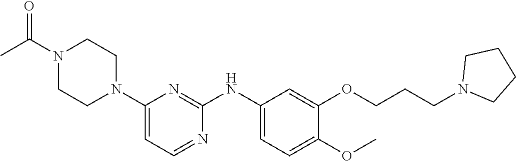 [SMILES: COc1ccc(Nc2nccc(N3CCN(C(C)=O)CC3)n2)cc1OCCCN1CCCC1] |
| 140 | 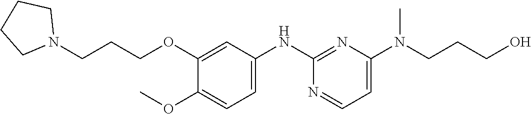 [SMILES: COc1ccc(Nc2nccc(N(C)CCCO)n2)cc1OCCCN1CCCC1] |
| 141 | 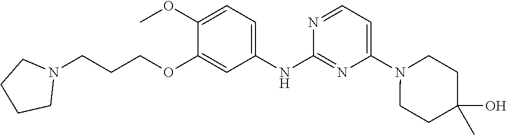 [SMILES: COc1ccc(Nc2nccc(N3CCC(C)(O)CC3)n2)cc1OCCCN1CCCC1] |
| 142 | 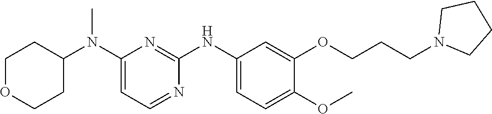 [SMILES: COc1ccc(Nc2nccc(N(C)C3CCOCC3)n2)cc1OCCCN1CCCC1] |
| 143 | 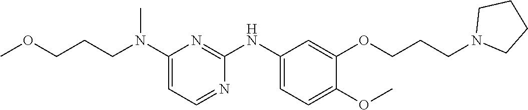 [SMILES: COCCCN(C)c1ccnc(Nc2ccc(OC)c(OCCCN3CCCC3)c2)n1] |
| 144 | 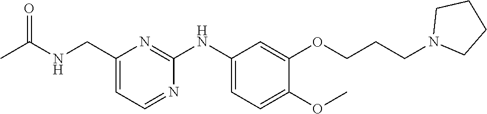 [SMILES: COc1ccc(Nc2nccc(CNC(C)=O)n2)cc1OCCCN1CCCC1] |
| 145 | 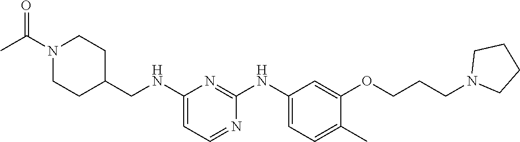 [SMILES: CC(=O)N1CCC(CNc2ccnc(Nc3ccc(C)c(OCCCN4CCCC4)c3)n2)CC1] |
| 146 | 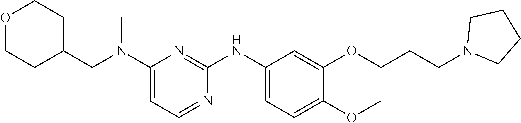 [SMILES: COc1ccc(Nc2nccc(N(C)CC3CCOCC3)n2)cc1OCCCN1CCCC1] |
| 147 | 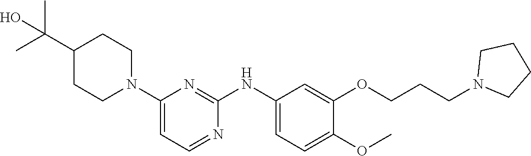 [SMILES: COc1ccc(Nc2nccc(N3CCC(C(C)(C)O)CC3)n2)cc1OCCCN1CCCC1] |
| 148 | 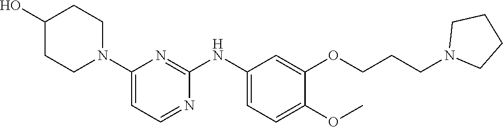 [SMILES: COc1ccc(Nc2nccc(N3CCC(O)CC3)n2)cc1OCCCN1CCCC1] |
| 149 | 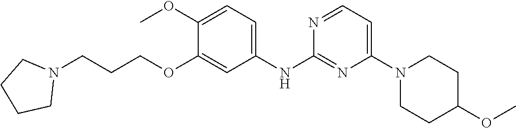 [SMILES: COc1ccc(Nc2nccc(N3CCC(OC)CC3)n2)cc1OCCCN1CCCC1] |
| 150 | 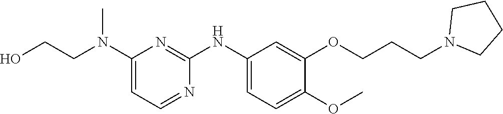 [SMILES: COc1ccc(Nc2nccc(N(C)CCO)n2)cc1OCCCN1CCCC1] |
| 151 | 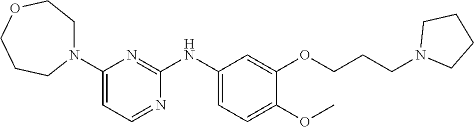 [SMILES: COc1ccc(Nc2nccc(N3CCCOCC3)n2)cc1OCCCN1CCCC1] |
| 152 | 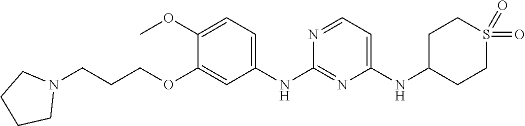 [SMILES: COc1ccc(Nc2nccc(NC3CCS(=O)(=O)CC3)n2)cc1OCCCN1CCCC1] |
| 153 | 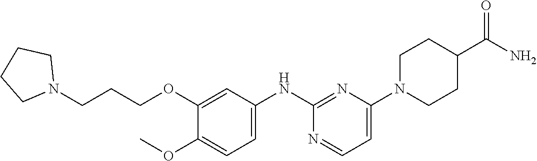 [SMILES: COc1ccc(Nc2nccc(N3CCC(C(N)=O)CC3)n2)cc1OCCCN1CCCC1] |
| 154 | 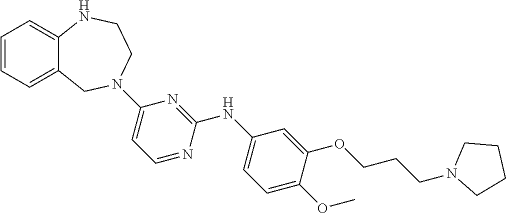 [SMILES: COc1ccc(Nc2nccc(N3CCNc4ccccc4C3)n2)cc1OCCCN1CCCC1] |
| 155 | 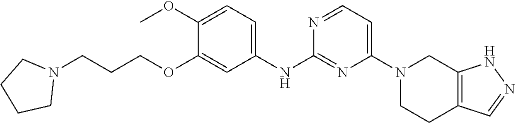 [SMILES: COc1ccc(Nc2nccc(N3CCc4cn[nH]c4C3)n2)cc1OCCCN1CCCC1] |
| 156 | 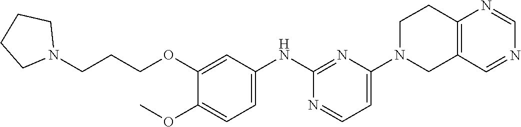 [SMILES: COc1ccc(Nc2nccc(N3CCc4ncncc4C3)n2)cc1OCCCN1CCCC1] |
| 157 | 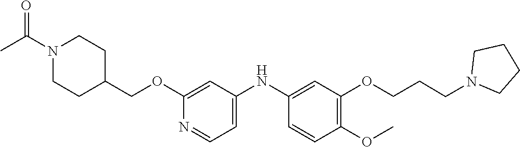 [SMILES: COc1ccc(Nc2ccnc(OCC3CCN(C(C)=O)CC3)c2)cc1OCCCN1CCCC1] |
| 158 | 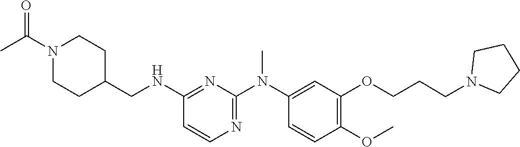 [SMILES: COc1ccc(N(C)c2nccc(NCC3CCN(C(C)=O)CC3)n2)cc1OCCCN1CCCC1] |
| 159 | 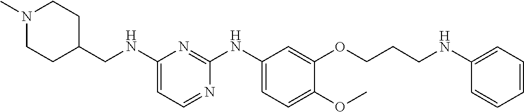 [SMILES: COc1ccc(Nc2nccc(NCC3CCN(C)CC3)n2)cc1OCCCNc1ccccc1] |
| 160 | 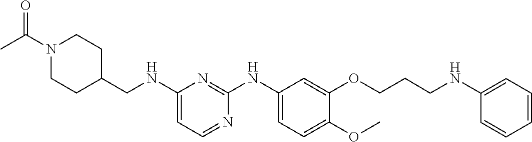 [SMILES: COc1ccc(Nc2nccc(NCC3CCN(C(C)=O)CC3)n2)cc1OCCCNc1ccccc1] |
| 161 | 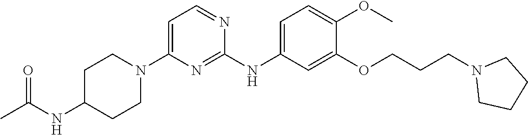 [SMILES: COc1ccc(Nc2nccc(N3CCC(NC(C)=O)CC3)n2)cc1OCCCN1CCCC1] |
| 162 | 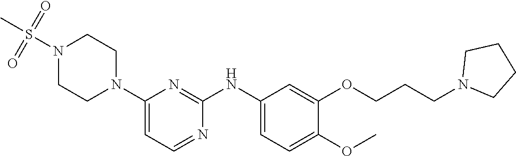 [SMILES: COc1ccc(Nc2nccc(N3CCN(S(C)(=O)=O)CC3)n2)cc1OCCCN1CCCC1] |
| 163 | 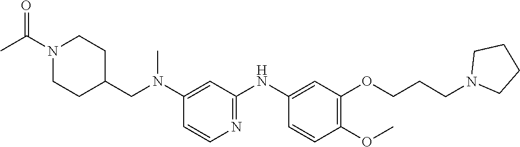 [SMILES: COc1ccc(Nc2cc(N(C)CC3CCN(C(C)=O)CC3)ccn2)cc1OCCCN1CCCC1] |
| 164 | 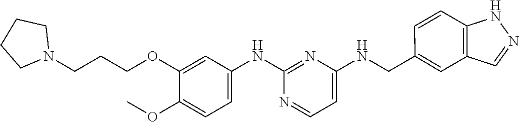 [SMILES: COc1ccc(Nc2nccc(NCc3ccc4[nH]ncc4c3)n2)cc1OCCCN1CCCC1] |
| 165 | 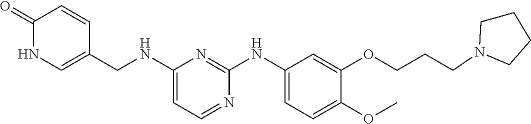 [SMILES: COc1ccc(Nc2nccc(NCc3ccc(=O)[nH]c3)n2)cc1OCCCN1CCCC1] |
| 166 | 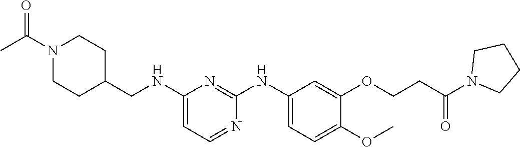 [SMILES: COc1ccc(Nc2nccc(NCC3CCN(C(C)=O)CC3)n2)cc1OCCC(=O)N1CCCC1] |
| 167 | 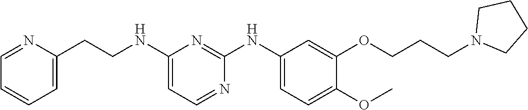 [SMILES: COc1ccc(Nc2nccc(NCCc3ccccn3)n2)cc1OCCCN1CCCC1] |
| 168 | 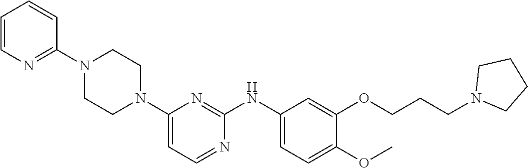 [SMILES: COc1ccc(Nc2nccc(N3CCN(c4ccccn4)CC3)n2)cc1OCCCN1CCCC1] |
| 169 | 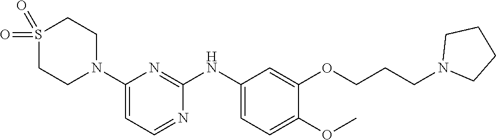 [SMILES: COc1ccc(Nc2nccc(N3CCS(=O)(=O)CC3)n2)cc1OCCCN1CCCC1] |
| 170 | 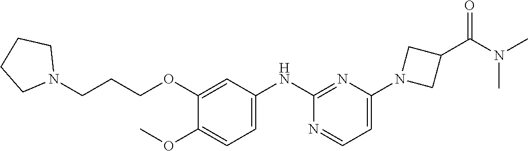 [SMILES: COc1ccc(Nc2nccc(N3CC(C(=O)N(C)C)C3)n2)cc1OCCCN1CCCC1] |
| 171 | 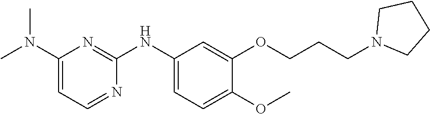 [SMILES: COc1ccc(Nc2nccc(N(C)C)n2)cc1OCCCN1CCCC1] |
| 172 | 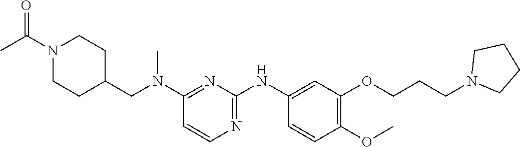 [SMILES: COc1ccc(Nc2nccc(N(C)CC3CCN(C(C)=O)CC3)n2)cc1OCCCN1CCCC1] |
| 173 | 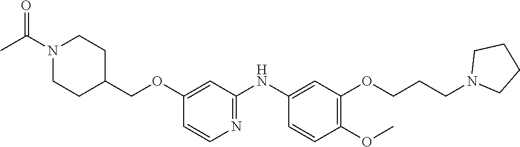 [SMILES: COc1ccc(Nc2cc(OCC3CCN(C(C)=O)CC3)ccn2)cc1OCCCN1CCCC1] |
| 174 | 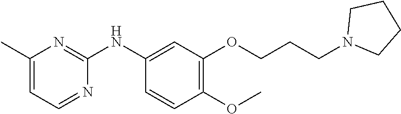 [SMILES: COc1ccc(Nc2nccc(C)n2)cc1OCCCN1CCCC1] |
| 175 | 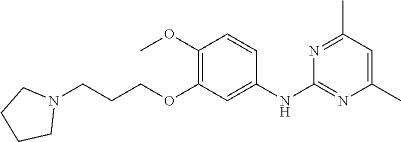 [SMILES: COc1ccc(Nc2nc(C)cc(C)n2)cc1OCCCN1CCCC1] |
| 176 | 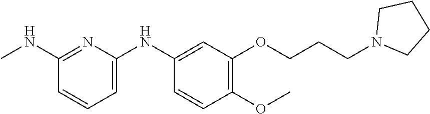 [SMILES: CNc1cccc(Nc2ccc(OC)c(OCCCN3CCCC3)c2)n1] |
| 177 | 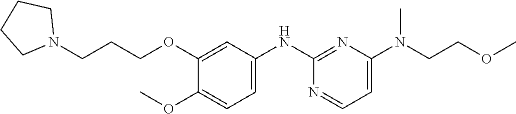 [SMILES: COCCN(C)c1ccnc(Nc2ccc(OC)c(OCCCN3CCCC3)c2)n1] |
| 178 | 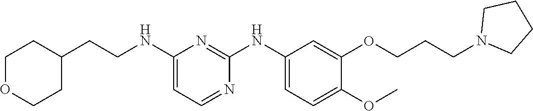 [SMILES: COc1ccc(Nc2nccc(NCCC3CCOCC3)n2)cc1OCCCN1CCCC1] |
| 179 | 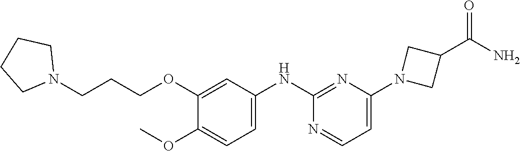 [SMILES: COc1ccc(Nc2nccc(N3CC(C(N)=O)C3)n2)cc1OCCCN1CCCC1] |
| 180 | 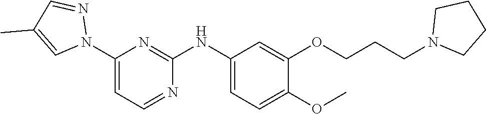 [SMILES: COc1ccc(Nc2nccc(-n3cc(C)cn3)n2)cc1OCCCN1CCCC1] |
| 181 | 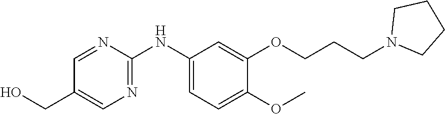 [SMILES: COc1ccc(Nc2ncc(CO)cn2)cc1OCCCN1CCCC1] |
| 182 | 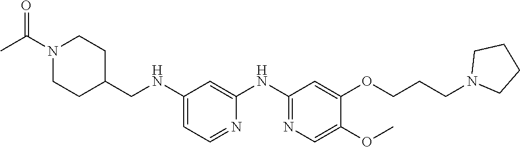 [SMILES: COc1cnc(Nc2cc(NCC3CCN(C(C)=O)CC3)ccn2)cc1OCCCN1CCCC1] |
| 183 | 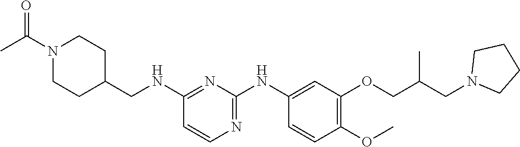 [SMILES: COc1ccc(Nc2nccc(NCC3CCN(C(C)=O)CC3)n2)cc1OCC(C)CN1CCCC1] |
| 184 | 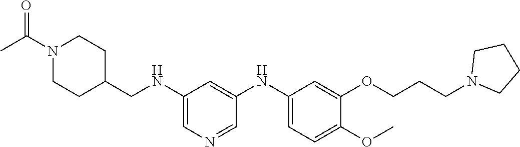 [SMILES: COc1ccc(Nc2cncc(NCC3CCN(C(C)=O)CC3)c2)cc1OCCCN1CCCC1] |
| 185 | 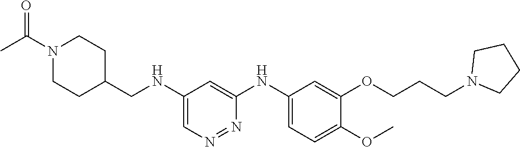 [SMILES: COc1ccc(Nc2cc(NCC3CCN(C(C)=O)CC3)cnn2)cc1OCCCN1CCCC1] |
| 186 | 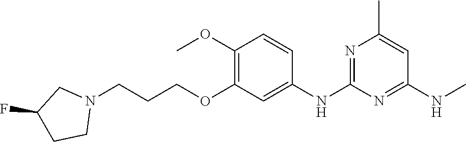 [SMILES: CNc1cc(C)nc(Nc2ccc(OC)c(OCCCN3CC[C@@H](F)C3)c2)n1] |
| 187 | 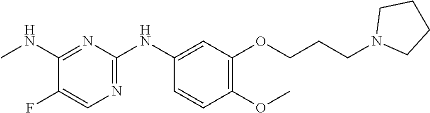 [SMILES: CNc1nc(Nc2ccc(OC)c(OCCCN3CCCC3)c2)ncc1F] |
| 188 | 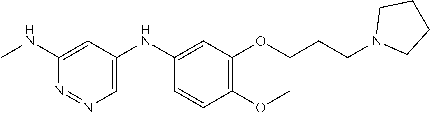 [SMILES: CNc1cc(Nc2ccc(OC)c(OCCCN3CCCC3)c2)cnn1] |
| 190 | 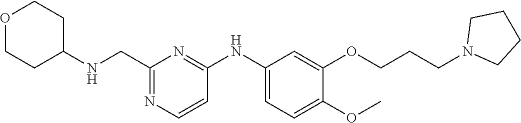 [SMILES: COc1ccc(Nc2ccnc(CNC3CCOCC3)n2)cc1OCCCN1CCCC1] |
| 191 | 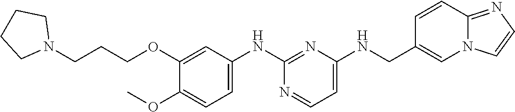 [SMILES: COc1ccc(Nc2nccc(NCc3ccc4nccn4c3)n2)cc1OCCCN1CCCC1] |
| 192 | 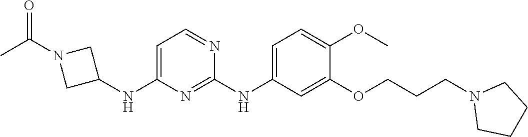 [SMILES: COc1ccc(Nc2nccc(NC3CN(C(C)=O)C3)n2)cc1OCCCN1CCCC1] |
| 193 | 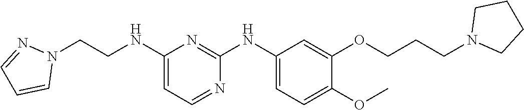 [SMILES: COc1ccc(Nc2nccc(NCCn3cccn3)n2)cc1OCCCN1CCCC1] |
| 194 | 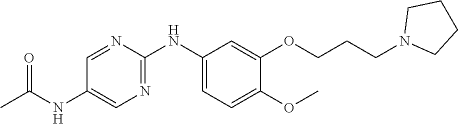 [SMILES: COc1ccc(Nc2ncc(NC(C)=O)cn2)cc1OCCCN1CCCC1] |
| 195 | 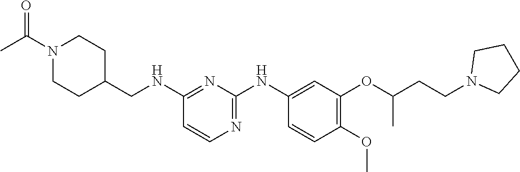 [SMILES: COc1ccc(Nc2nccc(NCC3CCN(C(C)=O)CC3)n2)cc1OC(C)CCN1CCCC1] |
| 196 | 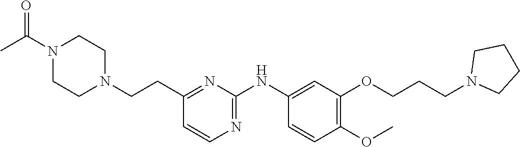 [SMILES: COc1ccc(Nc2nccc(CCN3CCN(C(C)=O)CC3)n2)cc1OCCCN1CCCC1] |
| 197 | 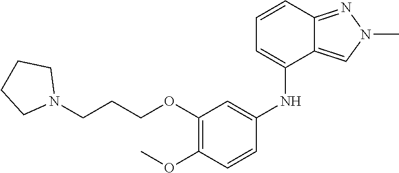 [SMILES: COc1ccc(Nc2cccc3nn(C)cc23)cc1OCCCN1CCCC1] |
| 199 | 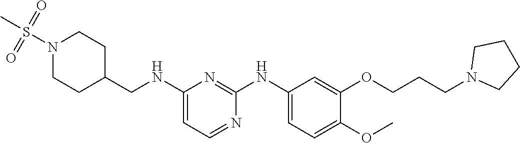 [SMILES: COc1ccc(Nc2nccc(NCC3CCN(S(C)(=O)=O)CC3)n2)cc1OCCCN1CCCC1] |
| 200 | 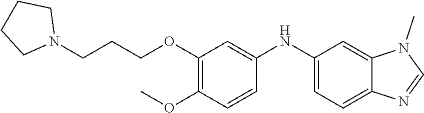 [SMILES: COc1ccc(Nc2ccc3ncn(C)c3c2)cc1OCCCN1CCCC1] |
| 201 | 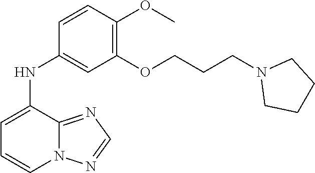 [SMILES: COc1ccc(Nc2cccn3ncnc23)cc1OCCCN1CCCC1] |
| 202 | 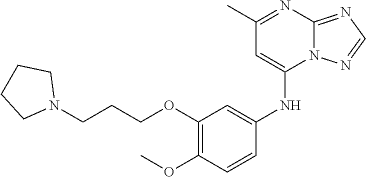 [SMILES: COc1ccc(Nc2cc(C)nc3ncnn23)cc1OCCCN1CCCC1] |
| 203 | 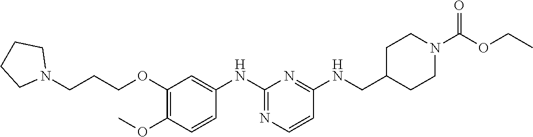 [SMILES: CCOC(=O)N1CCC(CNc2ccnc(Nc3ccc(OC)c(OCCCN4CCCC4)c3)n2)CC1] |
| 204 | 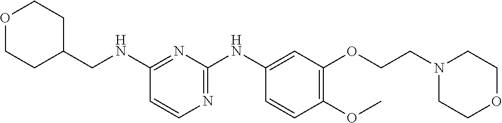 [SMILES: COc1ccc(Nc2nccc(NCC3CCOCC3)n2)cc1OCCN1CCOCC1] |
| 205 | 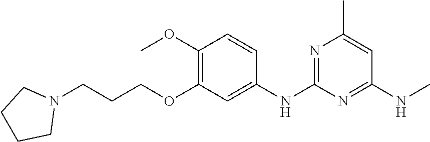 [SMILES: CNc1cc(C)nc(Nc2ccc(OC)c(OCCCN3CCCC3)c2)n1] |
| 206 | 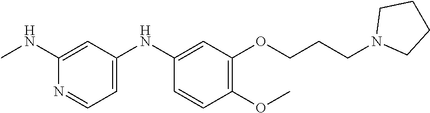 [SMILES: CNc1cc(Nc2ccc(OC)c(OCCCN3CCCC3)c2)ccn1] |
| 207 | 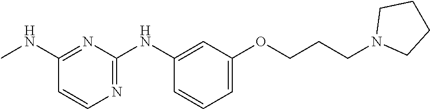 [SMILES: CNc1ccnc(Nc2cccc(OCCCN3CCCC3)c2)n1] |
| 208 | 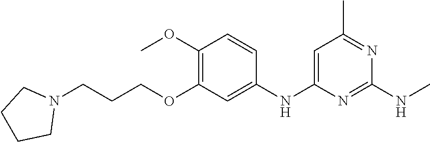 [SMILES: CNc1nc(C)cc(Nc2ccc(OC)c(OCCCN3CCCC3)c2)n1] |
| 209 | 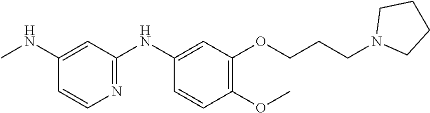 [SMILES: CNc1ccnc(Nc2ccc(OC)c(OCCCN3CCCC3)c2)c1] |
| 210 | 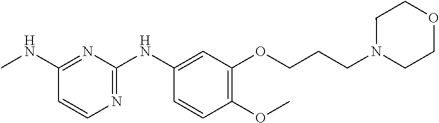 [SMILES: CNc1ccnc(Nc2ccc(OC)c(OCCCN3CCOCC3)c2)n1] |
| 211 | 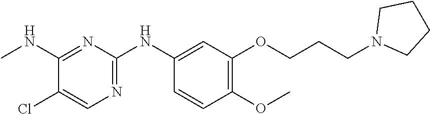 [SMILES: CNc1nc(Nc2ccc(OC)c(OCCCN3CCCC3)c2)ncc1Cl] |
| 212 | 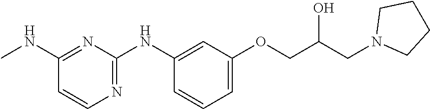 [SMILES: CNc1ccnc(Nc2cccc(OCC(O)CN3CCCC3)c2)n1] |
| 213 | 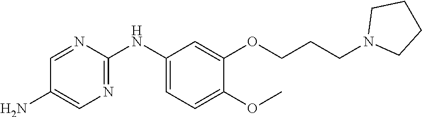 [SMILES: COc1ccc(Nc2ncc(N)cn2)cc1OCCCN1CCCC1] |
| 214 | 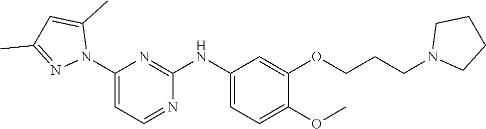 [SMILES: COc1ccc(Nc2nccc(-n3nc(C)cc3C)n2)cc1OCCCN1CCCC1] |
| 215 | 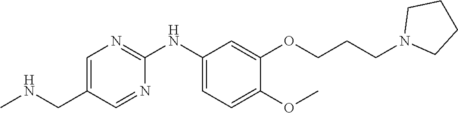 [SMILES: CNCc1cnc(Nc2ccc(OC)c(OCCCN3CCCC3)c2)nc1] |
| 216 | 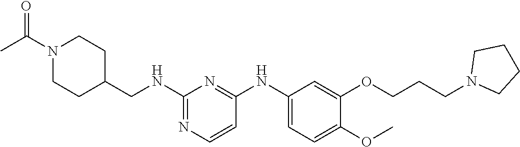 [SMILES: COc1ccc(Nc2ccnc(NCC3CCN(C(C)=O)CC3)n2)cc1OCCCN1CCCC1] |
| 217 | 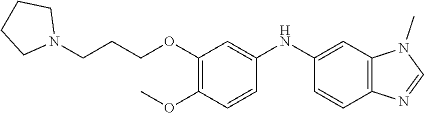 [SMILES: COc1ccc(Nc2ccc3ncn(C)c3c2)cc1OCCCN1CCCC1] |
| 218 | 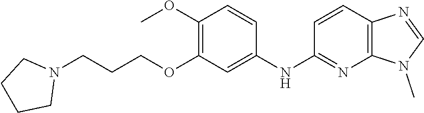 [SMILES: COc1ccc(Nc2ccc3ncn(C)c3n2)cc1OCCCN1CCCC1] |
| 219 | 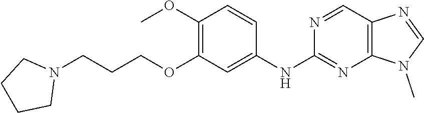 [SMILES: COc1ccc(Nc2ncc3ncn(C)c3n2)cc1OCCCN1CCCC1] |
| 220 | 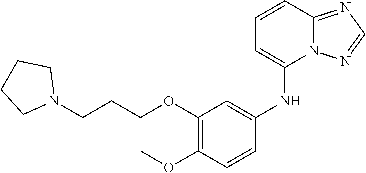 [SMILES: COc1ccc(Nc2cccc3ncnn23)cc1OCCCN1CCCC1] |
| 221 | 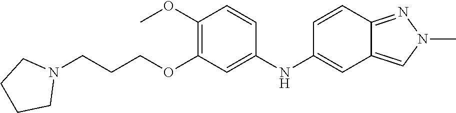 [SMILES: COc1ccc(Nc2ccc3nn(C)cc3c2)cc1OCCCN1CCCC1] |
| 222 | 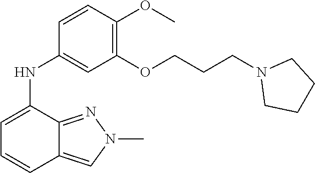 [SMILES: COc1ccc(Nc2cccc3cn(C)nc23)cc1OCCCN1CCCC1] |
| 223 | 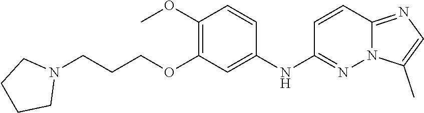 [SMILES: COc1ccc(Nc2ccc3ncc(C)n3n2)cc1OCCCN1CCCC1] |
| 224 | 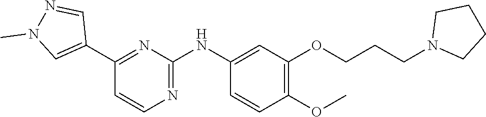 [SMILES: COc1ccc(Nc2nccc(-c3cnn(C)c3)n2)cc1OCCCN1CCCC1] |
| 225 | 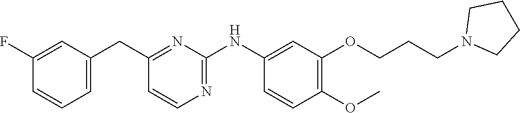 [SMILES: COc1ccc(Nc2nccc(Cc3cccc(F)c3)n2)cc1OCCCN1CCCC1] |
| 226 | 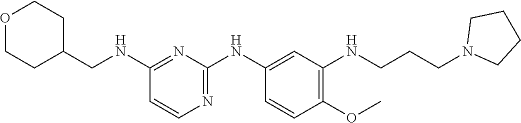 [SMILES: COc1ccc(Nc2nccc(NCC3CCOCC3)n2)cc1NCCCN1CCCC1] |
| 227 | 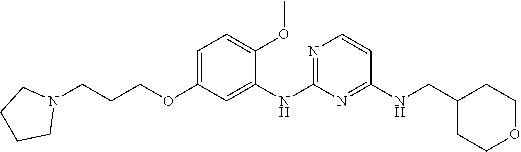 [SMILES: COc1ccc(OCCCN2CCCC2)cc1Nc1nccc(NCC2CCOCC2)n1] |
| 228 | 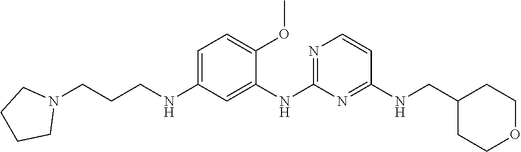 [SMILES: COc1ccc(NCCCN2CCCC2)cc1Nc1nccc(NCC2CCOCC2)n1] |
| 229 | 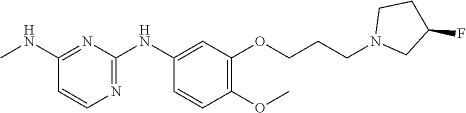 [SMILES: CNc1ccnc(Nc2ccc(OC)c(OCCCN3CC[C@@H](F)C3)c2)n1] |
| 230 | 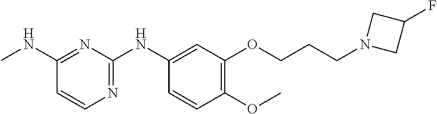 [SMILES: CNc1ccnc(Nc2ccc(OC)c(OCCCN3CC(F)C3)c2)n1] |
| 231 | 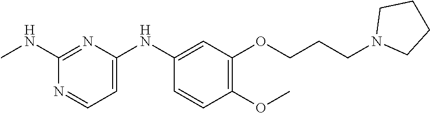 [SMILES: CNc1nccc(Nc2ccc(OC)c(OCCCN3CCCC3)c2)n1] |
| 232 | 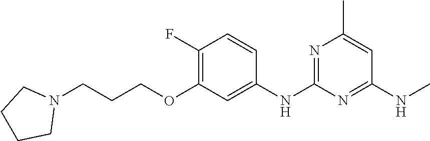 [SMILES: CNc1cc(C)nc(Nc2ccc(F)c(OCCCN3CCCC3)c2)n1] |
| 233 | 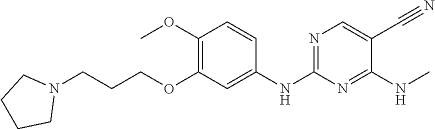 [SMILES: CNc1nc(Nc2ccc(OC)c(OCCCN3CCCC3)c2)ncc1C#N] |
| 234 | 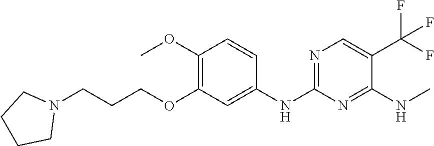 [SMILES: CNc1nc(Nc2ccc(OC)c(OCCCN3CCCC3)c2)ncc1C(F)(F)F] |
| 235 | 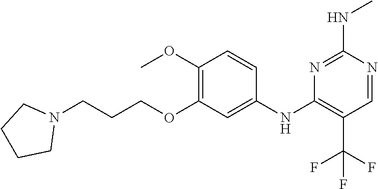 [SMILES: CNc1ncc(C(F)(F)F)c(Nc2ccc(OC)c(OCCCN3CCCC3)c2)n1] |
| 236 | 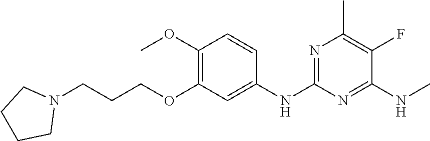 [SMILES: CNc1nc(Nc2ccc(OC)c(OCCCN3CCCC3)c2)nc(C)c1F] |
| 237 | 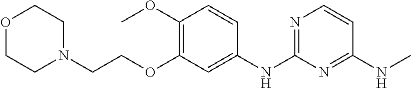 [SMILES: CNc1ccnc(Nc2ccc(OC)c(OCCN3CCOCC3)c2)n1] |
| 238 | 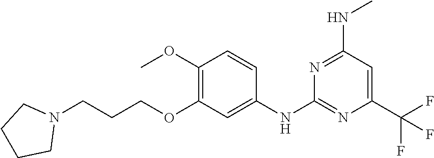 [SMILES: CNc1cc(C(F)(F)F)nc(Nc2ccc(OC)c(OCCCN3CCCC3)c2)n1] |
| 239 | 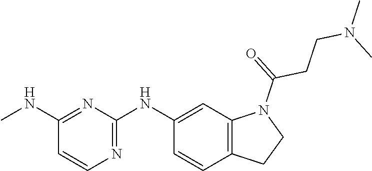 |
| 240 | 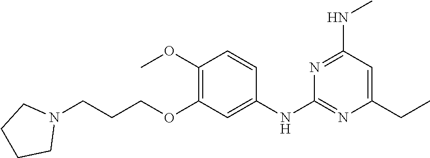 [SMILES: CCc1cc(NC)nc(Nc2ccc(OC)c(OCCCN3CCCC3)c2)n1] |
| 241 | 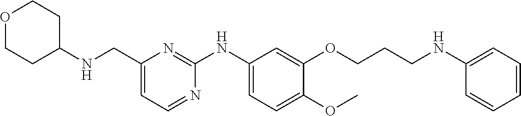 [SMILES: COc1ccc(Nc2nccc(CNC3CCOCC3)n2)cc1OCCCNc1ccccc1] |
| 242 | 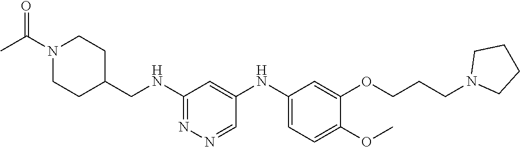 [SMILES: COc1ccc(Nc2cnnc(NCC3CCN(C(C)=O)CC3)c2)cc1OCCCN1CCCC1] |
| 243 | 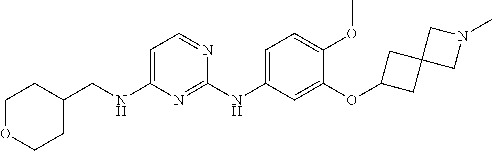 [SMILES: COc1ccc(Nc2nccc(NCC3CCOCC3)n2)cc1OC1CC2(C1)CN(C)C2] |
| 244 | 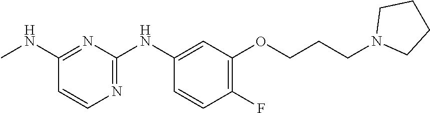 [SMILES: CNc1ccnc(Nc2ccc(F)c(OCCCN3CCCC3)c2)n1] |
| 245 | 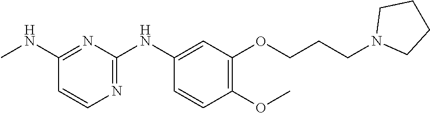 [SMILES: CNc1ccnc(Nc2ccc(OC)c(OCCCN3CCCC3)c2)n1] |
| 246 | 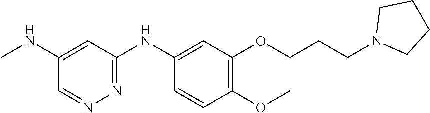 [SMILES: CNc1cnnc(Nc2ccc(OC)c(OCCCN3CCCC3)c2)c1] |
| 247 | 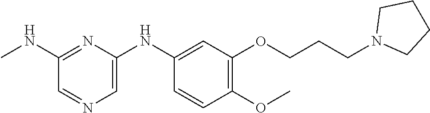 [SMILES: CNc1cncc(Nc2ccc(OC)c(OCCCN3CCCC3)c2)n1] |
| 248 | 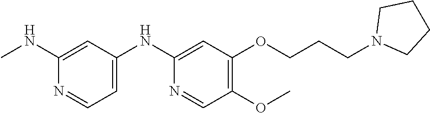 [SMILES: CNc1cc(Nc2cc(OCCCN3CCCC3)c(OC)cn2)ccn1] |
| 249 | 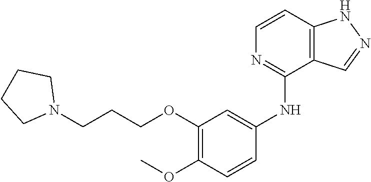 [SMILES: COc1ccc(Nc2nccc3[nH]ncc23)cc1OCCCN1CCCC1] |
| 250 | 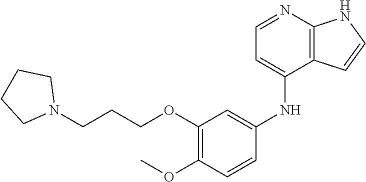 [SMILES: COc1ccc(Nc2ccnc3[nH]ccc23)cc1OCCCN1CCCC1] |
| 251 | 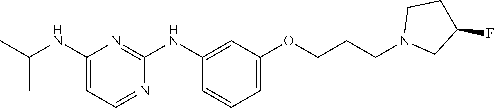 [SMILES: CC(C)Nc1ccnc(Nc2cccc(OCCCN3CC[C@@H](F)C3)c2)n1] |
| 252 | 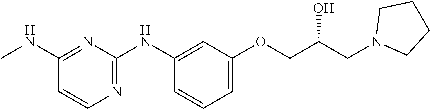 [SMILES: CNc1ccnc(Nc2cccc(OC[C@H](O)CN3CCCC3)c2)n1] |
| 253 | 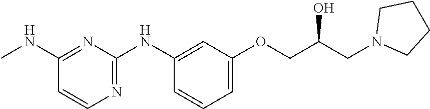 [SMILES: CNc1ccnc(Nc2cccc(OC[C@@H](O)CN3CCCC3)c2)n1] |
| 254 | 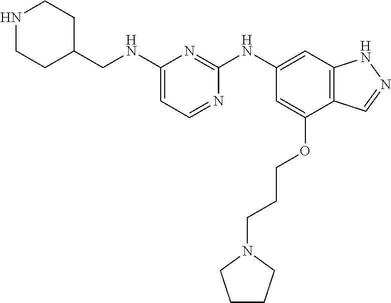 [SMILES: c1cc(NCC2CCNCC2)nc(Nc2cc(OCCCN3CCCC3)c3cn[nH]c3c2)n1] |
| 255 | 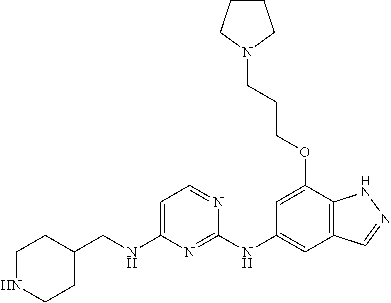 [SMILES: c1cc(NCC2CCNCC2)nc(Nc2cc(OCCCN3CCCC3)c3[nH]ncc3c2)n1] |
| 256 | 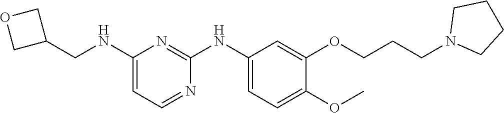 [SMILES: COc1ccc(Nc2nccc(NCC3COC3)n2)cc1OCCCN1CCCC1] |
| 257 | 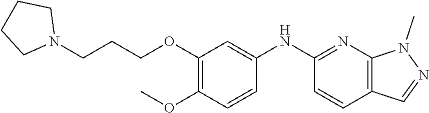 [SMILES: COc1ccc(Nc2ccc3cnn(C)c3n2)cc1OCCCN1CCCC1] |
| 258 | 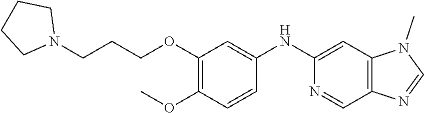 [SMILES: COc1ccc(Nc2cc3c(cn2)ncn3C)cc1OCCCN1CCCC1] |
| 259 | 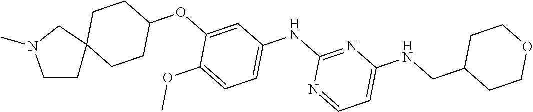 [SMILES: COc1ccc(Nc2nccc(NCC3CCOCC3)n2)cc1OC1CCC2(CC1)CCN(C)C2] |
| 260 | 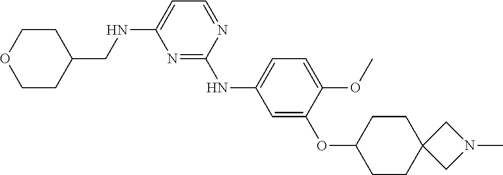 [SMILES: COc1ccc(Nc2nccc(NCC3CCOCC3)n2)cc1OC1CCC2(CC1)CN(C)C2] |
| 261 | 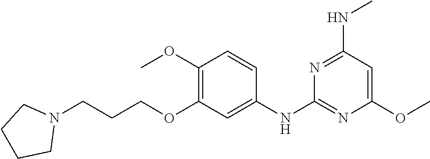 [SMILES: CNc1cc(OC)nc(Nc2ccc(OC)c(OCCCN3CCCC3)c2)n1] |
| 262a | 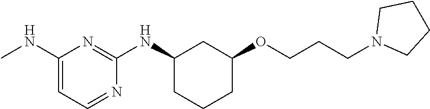 [SMILES: CNc1ccnc(N[C@@H]2CCC[C@H](OCCCN3CCCC3)C2)n1] |
| 262b | 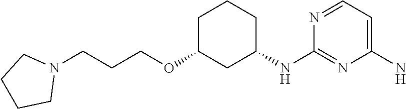 [SMILES: Nc1ccnc(N[C@H]2CCC[C@@H](OCCCN3CCCC3)C2)n1] |
| 263 | 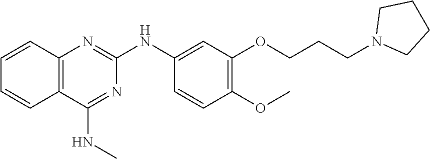 [SMILES: CNc1nc(Nc2ccc(OC)c(OCCCN3CCCC3)c2)nc2ccccc12] |
| 264 | 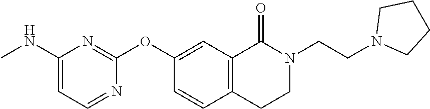 [SMILES: CNc1ccnc(Oc2ccc3c(c2)C(=O)N(CCN2CCCC2)CC3)n1] |
| 265 | 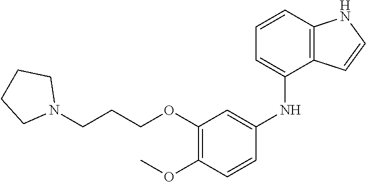 [SMILES: COc1ccc(Nc2cccc3[nH]ccc23)cc1OCCCN1CCCC1] |
| 266 | 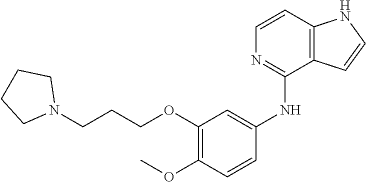 [SMILES: COc1ccc(Nc2nccc3[nH]ccc23)cc1OCCCN1CCCC1] |
| 267 | 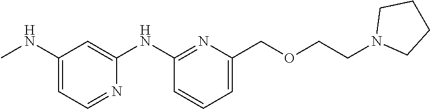 [SMILES: CNc1ccnc(Nc2cccc(COCCN3CCCC3)n2)c1] |
| 268 | 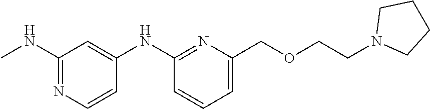 [SMILES: CNc1cc(Nc2cccc(COCCN3CCCC3)n2)ccn1] |
| 269 | 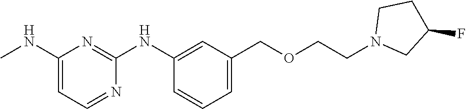 [SMILES: CNc1ccnc(Nc2cccc(COCCN3CC[C@@H](F)C3)c2)n1] |
| 271 | 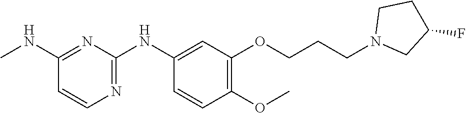 [SMILES: CNc1ccnc(Nc2ccc(OC)c(OCCCN3CC[C@H](F)C3)c2)n1] |
| 272 | 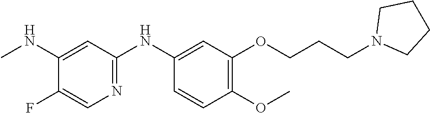 [SMILES: CNc1cc(Nc2ccc(OC)c(OCCCN3CCCC3)c2)ncc1F] |
| 273 | 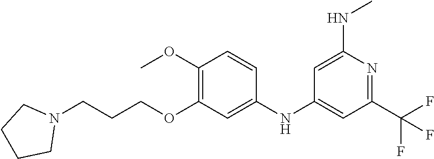 [SMILES: CNc1cc(Nc2ccc(OC)c(OCCCN3CCCC3)c2)cc(C(F)(F)F)n1] |
| 274 | 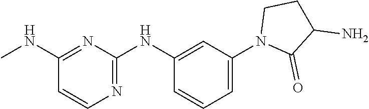 [SMILES: CNc1ccnc(Nc2cccc(N3CCC(N)C3=O)c2)n1] |
| 275 | 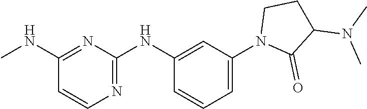 |
| 276 | 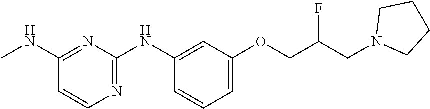 [SMILES: CNc1ccnc(Nc2cccc(OCC(F)CN3CCCC3)c2)n1] |
| 277 | 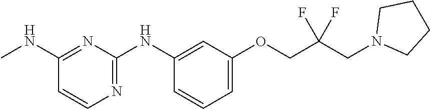 [SMILES: CNc1ccnc(Nc2cccc(OCC(F)(F)CN3CCCC3)c2)n1] |
| 278 | 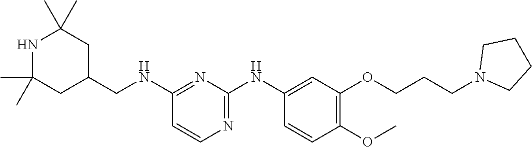 [SMILES: COc1ccc(Nc2nccc(NCC3CC(C)(C)NC(C)(C)C3)n2)cc1OCCCN1CCCC1] |
| 279 | 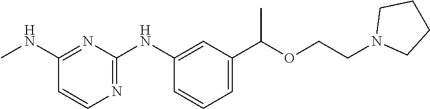 [SMILES: CNc1ccnc(Nc2cccc(C(C)OCCN3CCCC3)c2)n1] |
| 280 | 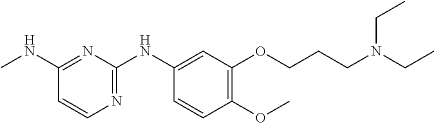 [SMILES: CCN(CC)CCCOc1cc(Nc2nccc(NC)n2)ccc1OC] |
| 281 | 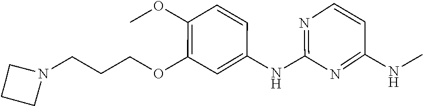 [SMILES: CNc1ccnc(Nc2ccc(OC)c(OCCCN3CCCC3)c2)n1] |
| 282 | 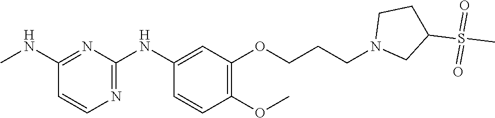 |
| 283 | 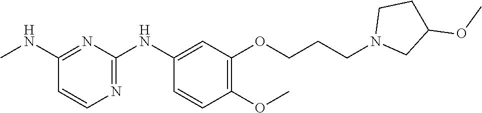 |
| 284 | 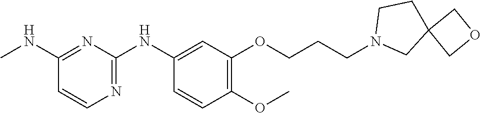 [SMILES: CNc1ccnc(Nc2ccc(OC)c(OCCCN3CCC4(COC4)C3)c2)n1] |
| 285 | 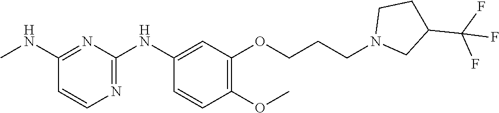 [SMILES: CNc1ccnc(Nc2ccc(OC)c(OCCCN3CCC(C(F)(F)F)C3)c2)n1] |
| 286 | 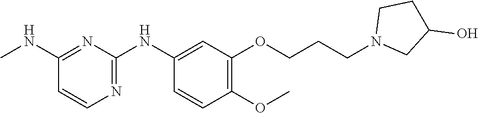 [SMILES: CNc1ccnc(Nc2ccc(OC)c(OCCCN3CCC(O)C3)c2)n1] |
| 287 | 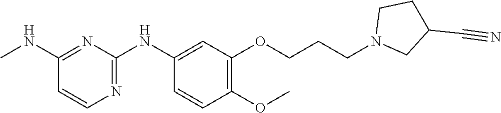 [SMILES: CNc1ccnc(Nc2ccc(OC)c(OCCCN3CCC(C#N)C3)c2)n1] |
| 288 | 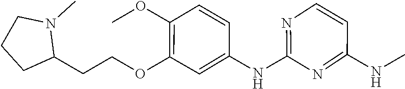 [SMILES: CNc1ccnc(Nc2ccc(OC)c(OCCC3CCCN3C)c2)n1] |
| 289 | 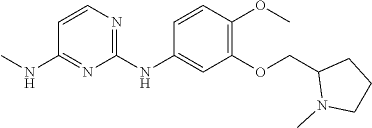 [SMILES: CNc1ccnc(Nc2ccc(OC)c(OCC3CCCN3C)c2)n1] |
| 290 | 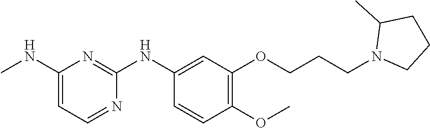 [SMILES: CNc1ccnc(Nc2ccc(OC)c(OCCCN3CCCC3C)c2)n1] |
| 291 | 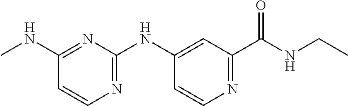 [SMILES: CCNC(=O)c1cc(Nc2nccc(NC)n2)ccn1] |
| 292 | 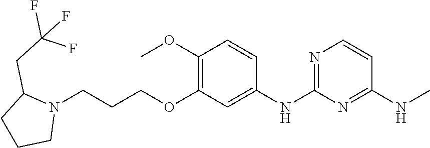 [SMILES: CNc1ccnc(Nc2ccc(OC)c(OCCCN3CCCC3CC(F)(F)F)c2)n1] |
| 293 | 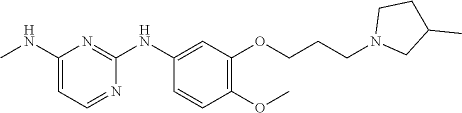 |
| 294 | 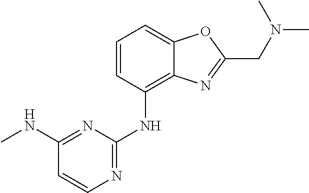 |
| 295 | 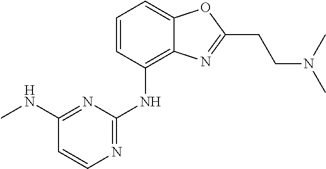 |
| 296 | 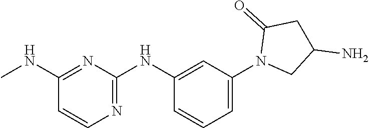 [SMILES: CNc1ccnc(Nc2cccc(N3CC(N)CC3=O)c2)n1] |
| 297 | 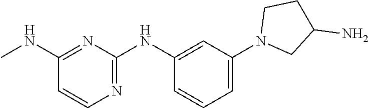 [SMILES: CNc1ccnc(Nc2cccc(N3CCC(N)C3)c2)n1] |
| 298 | 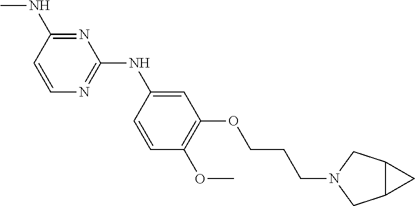 [SMILES: CNc1ccnc(Nc2ccc(OC)c(OCCCN3CC4CC4C3)c2)n1] |
| 299 | 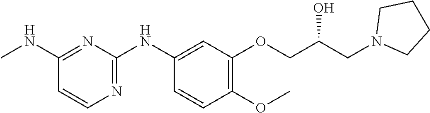 [SMILES: CNc1ccnc(Nc2ccc(OC)c(OC[C@H](O)CN3CCCC3)c2)n1] |
| 300 | 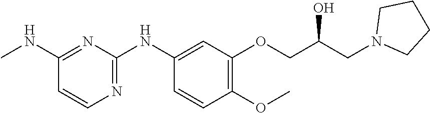 [SMILES: CNc1ccnc(Nc2ccc(OC)c(OC[C@@H](O)CN3CCCC3)c2)n1] |
| 301 | 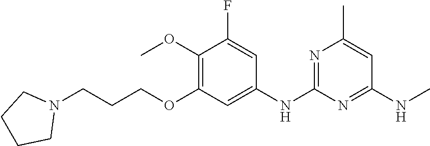 [SMILES: CNc1cc(C)nc(Nc2cc(F)c(OC)c(OCCCN3CCCC3)c2)n1] |
| 302 | 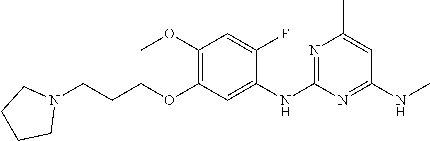 [SMILES: CNc1cc(C)nc(Nc2cc(OCCCN3CCCC3)c(OC)cc2F)n1] |
| 303 | 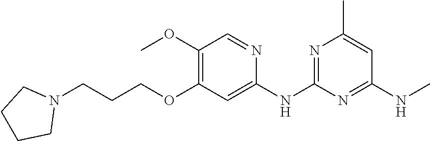 [SMILES: CNc1cc(C)nc(Nc2cc(OCCCN3CCCC3)c(OC)cn2)n1] |
| 304 | 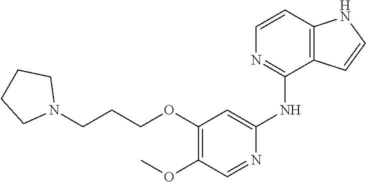 |
| 305 | 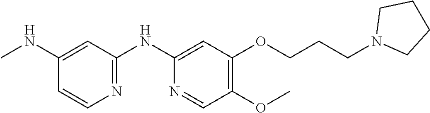 [SMILES: CNc1ccnc(Nc2cc(OCCCN3CCCC3)c(OC)cn2)c1] |
| 306 | 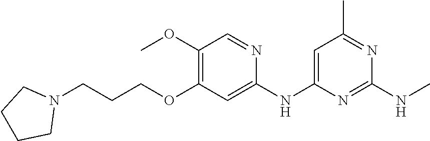 [SMILES: CNc1nc(C)cc(Nc2cc(OCCCN3CCCC3)c(OC)cn2)n1] |
| 307 | 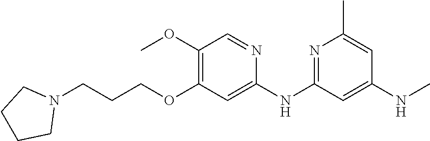 [SMILES: CNc1cc(C)nc(Nc2cc(OCCCN3CCCC3)c(OC)cn2)c1] |
| 308 | 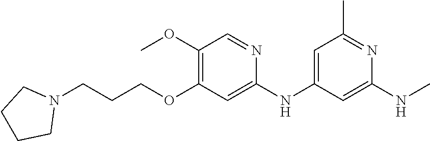 [SMILES: CNc1cc(Nc2cc(OCCCN3CCCC3)c(OC)cn2)cc(C)n1] |
| 309 | 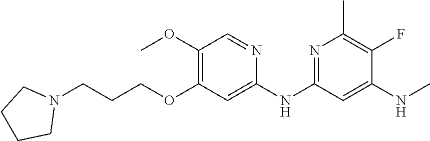 [SMILES: CNc1cc(Nc2cc(OCCCN3CCCC3)c(OC)cn2)nc(C)c1F] |
| 310 | 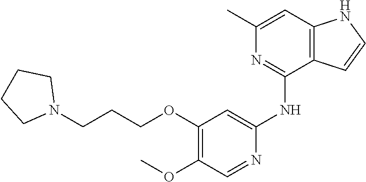 [SMILES: COc1cnc(Nc2nc(C)cc3[nH]ccc23)cc1OCCCN1CCCC1] |
| 311 | 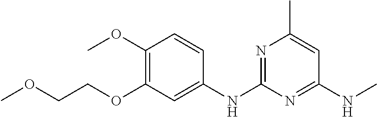 |
| 312 | 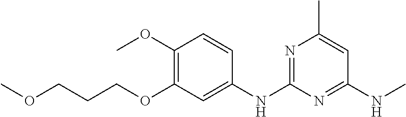 |
| 313 | 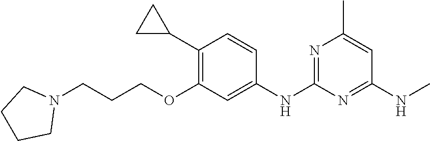 [SMILES: CNc1cc(C)nc(Nc2ccc(C3CC3)c(OCCCN3CCCC3)c2)n1] |
| 314 | 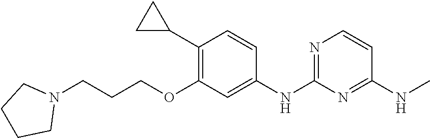 [SMILES: CNc1ccnc(Nc2ccc(C3CC3)c(OCCCN3CCCC3)c2)n1] |
| 315 | 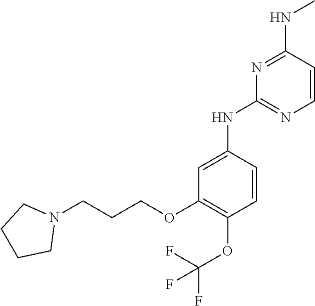 [SMILES: CNc1ccnc(Nc2ccc(OC(F)(F)F)c(OCCCN3CCCC3)c2)n1] |
| 316 | 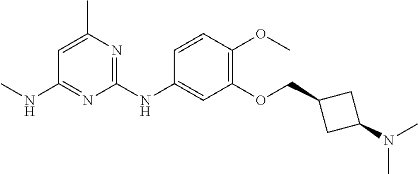 |
| 317 | 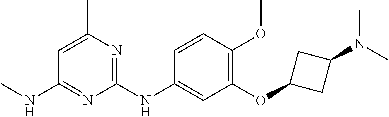 |
| 318 | 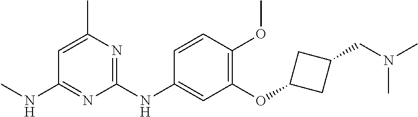 |
| 319 | 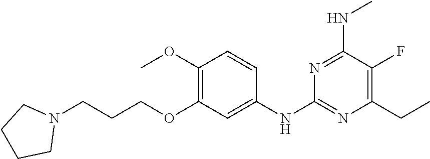 [SMILES: CCc1nc(Nc2ccc(OC)c(OCCCN3CCCC3)c2)nc(NC)c1F] |
| 320 | 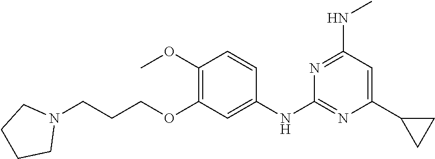 [SMILES: CNc1cc(C2CC2)nc(Nc2ccc(OC)c(OCCCN3CCCC3)c2)n1] |
| 321 | 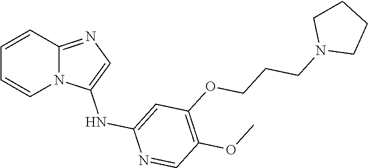 [SMILES: COc1cnc(Nc2cnc3ccccn23)cc1OCCCN1CCCC1] |
| 322 | 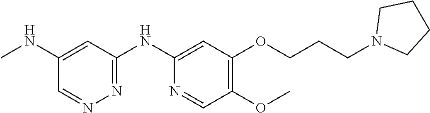 |
| 323 | 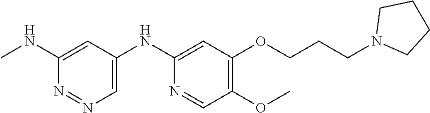 |
| 324 | 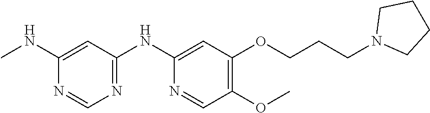 [SMILES: CNc1cc(Nc2cc(OCCCN3CCCC3)c(OC)cn2)ncn1] |
| 325 | 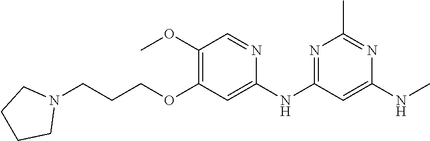 [SMILES: CNc1cc(Nc2cc(OCCCN3CCCC3)c(OC)cn2)nc(C)n1] |
| 326 | 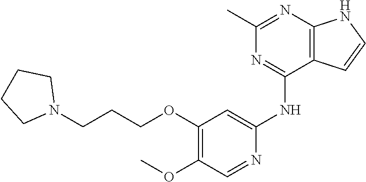 [SMILES: COc1cnc(Nc2nc(C)nc3[nH]ccc23)cc1OCCCN1CCCC1] |
| 327 | 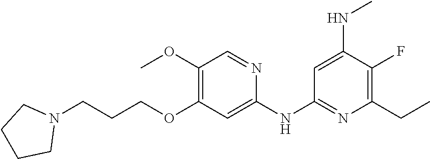 [SMILES: CCc1nc(Nc2cc(OCCCN3CCCC3)c(OC)cn2)cc(NC)c1F] |
| 328 | 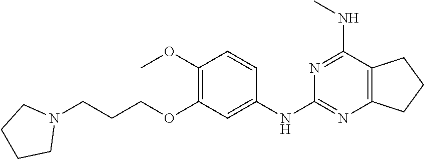 [SMILES: CNc1nc(Nc2ccc(OC)c(OCCCN3CCCC3)c2)nc2c1CCC2] |
| 329 | 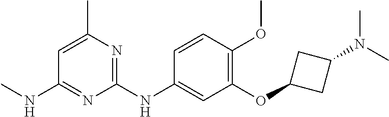 |
| 330 | 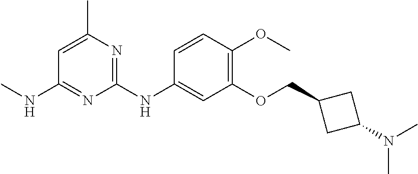 |
| 331 | 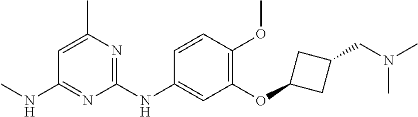 |
| 332 | 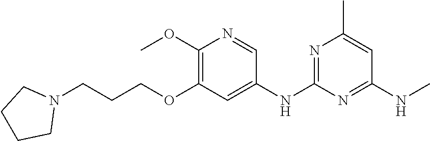 [SMILES: CNc1cc(C)nc(Nc2cnc(OC)c(OCCCN3CCCC3)c2)n1] |
| 333 | 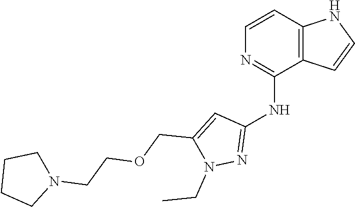 [SMILES: CCn1nc(Nc2nccc3[nH]ccc23)cc1COCCN1CCCC1] |
| 334 | 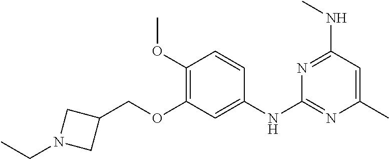 [SMILES: CCN1CC(COc2cc(Nc3nc(C)cc(NC)n3)ccc2OC)C1] |
| 335 | 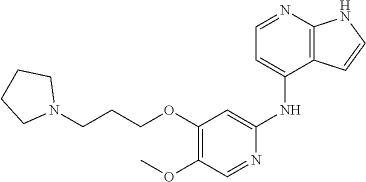 [SMILES: COc1cnc(Nc2ccnc3[nH]ccc23)cc1OCCCN1CCCC1] |
| 336 | 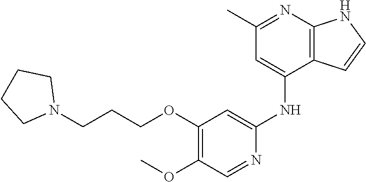 [SMILES: COc1cnc(Nc2cc(C)nc3[nH]ccc23)cc1OCCCN1CCCC1] |
| 337 | 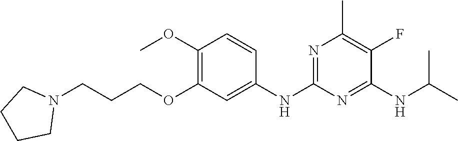 [SMILES: COc1ccc(Nc2nc(C)c(F)c(NC(C)C)n2)cc1OCCCN1CCCC1] |

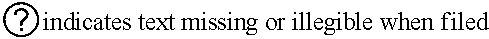

TABLE 1B

| Cmpd. | |
|---|---|
| No. | Structure |
| 338 | 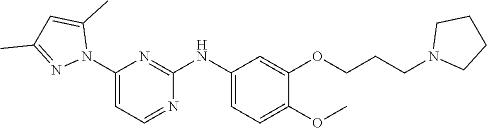 [SMILES: COc1ccc(Nc2nccc(-n3nc(C)cc3C)n2)cc1OCCCN1CCCC1] |
| 339 | 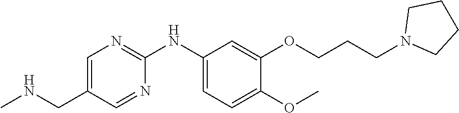 [SMILES: CNCc1cnc(Nc2ccc(OC)c(OCCCN3CCCC3)c2)nc1] |
| 340 | 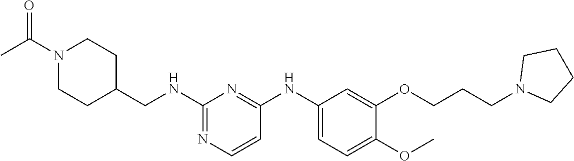 [SMILES: COc1ccc(Nc2ccnc(NCC3CCN(C(C)=O)CC3)n2)cc1OCCCN1CCCC1] |
| 341 | 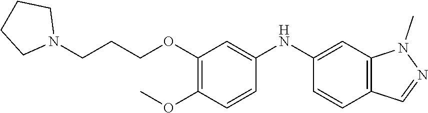 [SMILES: COc1ccc(Nc2ccc3cnn(C)c3c2)cc1OCCCN1CCCC1] |
| 342 | 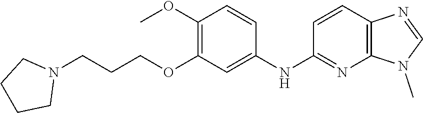 [SMILES: COc1ccc(Nc2ccc3ncn(C)c3n2)cc1OCCCN1CCCC1] |
| 343 | 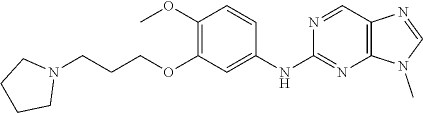 [SMILES: COc1ccc(Nc2ncc3ncn(C)c3n2)cc1OCCCN1CCCC1] |
| 344 | 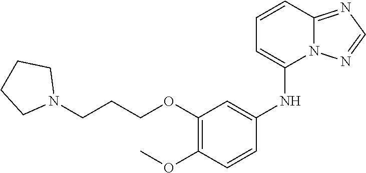 [SMILES: COc1ccc(Nc2cccc3ncnn23)cc1OCCCN1CCCC1] |
| 345 | 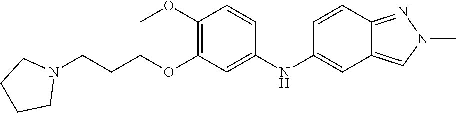 [SMILES: COc1ccc(Nc2ccc3nn(C)cc3c2)cc1OCCCN1CCCC1] |
| 346 | 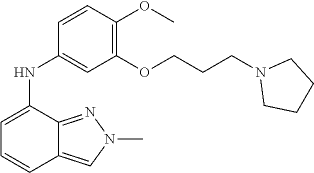 [SMILES: COc1ccc(Nc2cccc3cn(C)nc23)cc1OCCCN1CCCC1] |
| 347 | 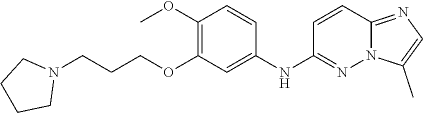 [SMILES: COc1ccc(Nc2ccc3ncc(C)n3n2)cc1OCCCN1CCCC1] |
| 348 | 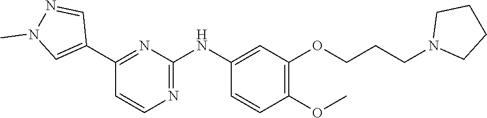 [SMILES: COc1ccc(Nc2nccc(-c3cnn(C)c3)n2)cc1OCCCN1CCCC1] |
| 349 | 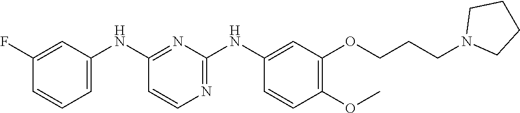 [SMILES: COc1ccc(Nc2nccc(Nc3cccc(F)c3)n2)cc1OCCCN1CCCC1] |
| 350 | 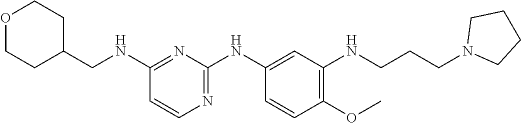 [SMILES: COc1ccc(Nc2nccc(NCC3CCOCC3)n2)cc1NCCCN1CCCC1] |
| 351 | 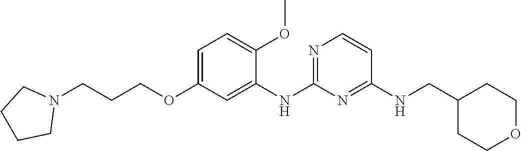 [SMILES: COc1ccc(OCCCN2CCCC2)cc1Nc1nccc(NCC2CCOCC2)n1] |
| 352 | 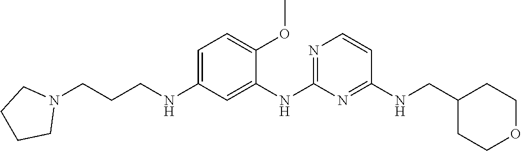 [SMILES: COc1ccc(NCCCN2CCCC2)cc1Nc1nccc(NCC2CCOCC2)n1] |
| 353 | 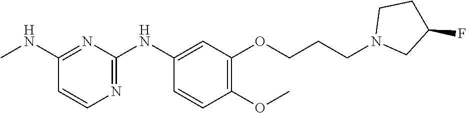 [SMILES: CNc1ccnc(Nc2ccc(OC)c(OCCCN3CC[C@@H](F)C3)c2)n1] |
| 354 | 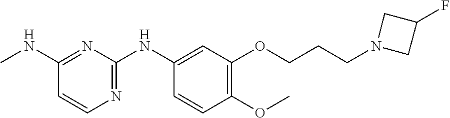 [SMILES: CNc1ccnc(Nc2ccc(OC)c(OCCCN3CC(F)C3)c2)n1] |
| 355 | 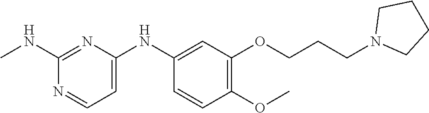 [SMILES: CNc1nccc(Nc2ccc(OC)c(OCCCN3CCCC3)c2)n1] |
| 356 | 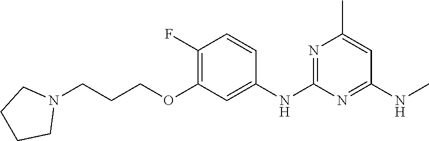 [SMILES: CNc1cc(C)nc(Nc2ccc(F)c(OCCCN3CCCC3)c2)n1] |
| 357 | 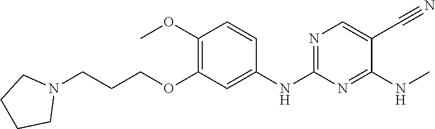 [SMILES: CNc1nc(Nc2ccc(OC)c(OCCCN3CCCC3)c2)ncc1C#N] |
| 358 | 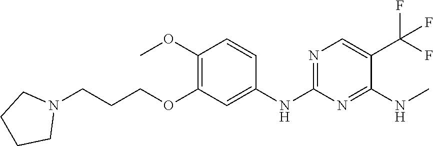 [SMILES: CNc1nc(Nc2ccc(OC)c(OCCCN3CCCC3)c2)ncc1C(F)(F)F] |
| 359 | 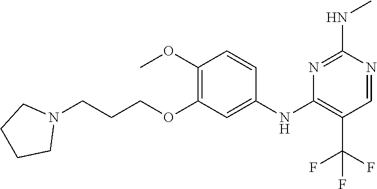 [SMILES: CNc1ncc(C(F)(F)F)c(Nc2ccc(OC)c(OCCCN3CCCC3)c2)n1] |
| 360 | 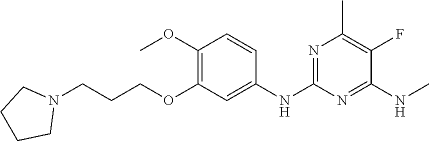 [SMILES: CNc1nc(Nc2ccc(OC)c(OCCCN3CCCC3)c2)nc(C)c1F] |
| 361 | 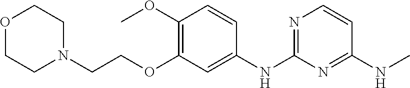 [SMILES: CNc1ccnc(Nc2ccc(OC)c(OCCN3CCOCC3)c2)n1] |
| 362 | 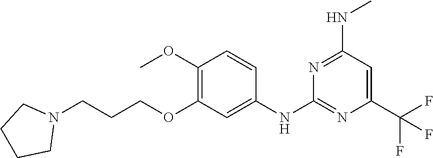 [SMILES: CNc1cc(C(F)(F)F)nc(Nc2ccc(OC)c(OCCCN3CCCC3)c2)n1] |
| 363 | 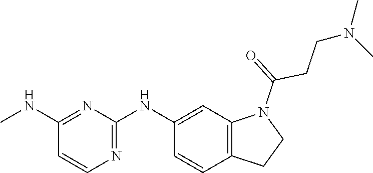 |
| 364 | 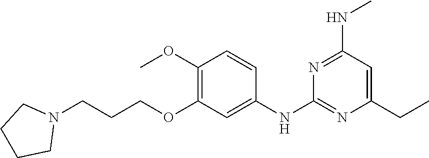 [SMILES: CCc1cc(NC)nc(Nc2ccc(OC)c(OCCCN3CCCC3)c2)n1] |
| 365 | 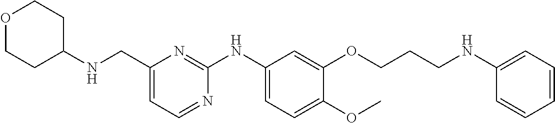 [SMILES: COc1ccc(Nc2nccc(CNC3CCOCC3)n2)cc1OCCCNc1ccccc1] |
| 366 | 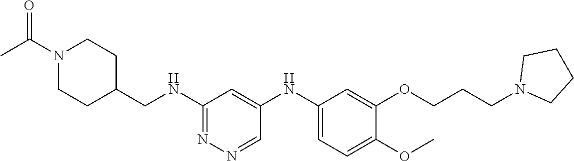 [SMILES: COc1ccc(Nc2cnnc(NCC3CCN(C(C)=O)CC3)c2)cc1OCCCN1CCCC1] |
| 367 | 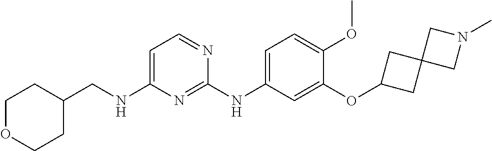 [SMILES: COc1ccc(Nc2nccc(NCC3CCOCC3)n2)cc1OC1CC2(C1)CN(C)C2] |
| 368 | 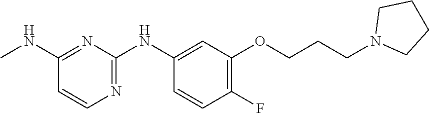 [SMILES: CNc1ccnc(Nc2ccc(F)c(OCCCN3CCCC3)c2)n1] |
| 369 | 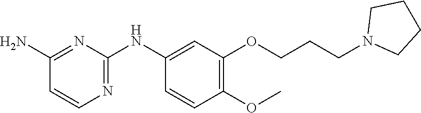 [SMILES: COc1ccc(Nc2nccc(N)n2)cc1OCCCN1CCCC1] |
| 370 | 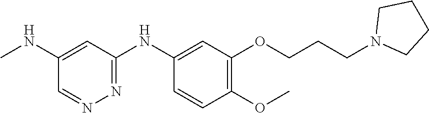 [SMILES: CNc1cnnc(Nc2ccc(OC)c(OCCCN3CCCC3)c2)c1] |
| 371 | 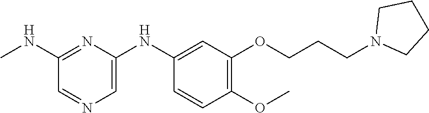 [SMILES: CNc1cncc(Nc2ccc(OC)c(OCCCN3CCCC3)c2)n1] |
| 372 | 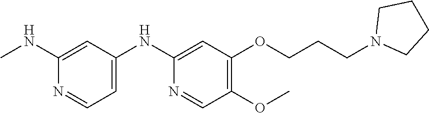 [SMILES: CNc1cc(Nc2cc(OCCCN3CCCC3)c(OC)cn2)ccn1] |
| 373 | 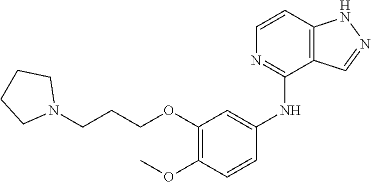 [SMILES: COc1ccc(Nc2nccc3[nH]ncc23)cc1OCCCN1CCCC1] |
| 374 | 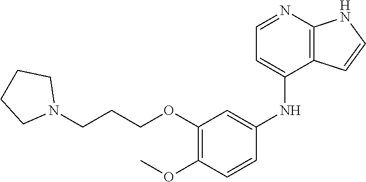 [SMILES: COc1ccc(Nc2ccnc3[nH]ccc23)cc1OCCCN1CCCC1] |
| 375 | 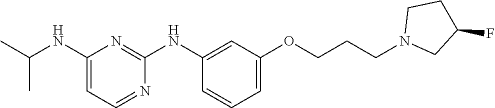 [SMILES: CC(C)Nc1ccnc(Nc2cccc(OCCCN3CC[C@@H](F)C3)c2)n1] |
| 376 | 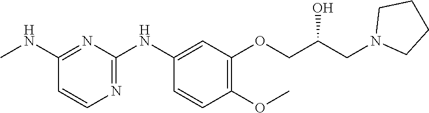 [SMILES: CNc1ccnc(Nc2ccc(OC)c(OC[C@H](O)CN3CCCC3)c2)n1] |
| 377 | 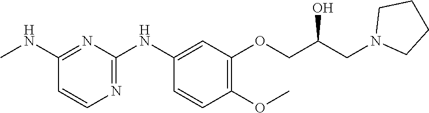 [SMILES: CNc1ccnc(Nc2ccc(OC)c(OC[C@@H](O)CN3CCCC3)c2)n1] |
| 378 | 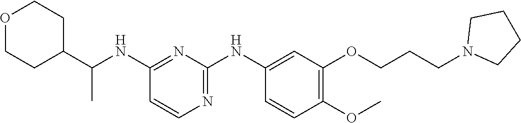 [SMILES: COc1ccc(Nc2nccc(NC(C)C3CCOCC3)n2)cc1OCCCN1CCCC1] |
| 379 | 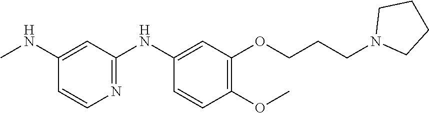 [SMILES: CNc1ccnc(Nc2ccc(OC)c(OCCCN3CCCC3)c2)c1] |
| 380 | 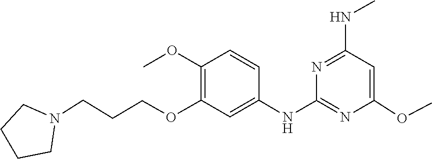 [SMILES: CNc1cc(OC)nc(Nc2ccc(OC)c(OCCCN3CCCC3)c2)n1] |
| 381 | 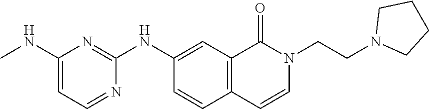 [SMILES: CNc1ccnc(Nc2ccc3ccn(CCN4CCCC4)c(=O)c3c2)n1] |
| 382 | 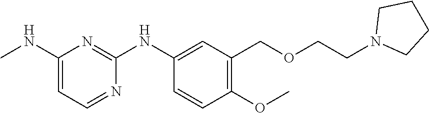 [SMILES: CNc1ccnc(Nc2ccc(OC)c(COCCN3CCCC3)c2)n1] |
| 383 | 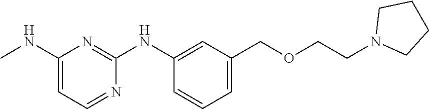 [SMILES: CNc1ccnc(Nc2cccc(COCCN3CCCC3)c2)n1] |
| 384 | 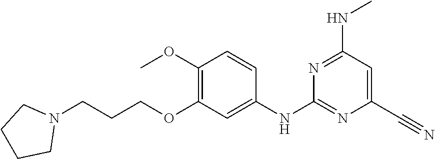 [SMILES: CNc1cc(C#N)nc(Nc2ccc(OC)c(OCCCN3CCCC3)c2)n1] |
| 385 | 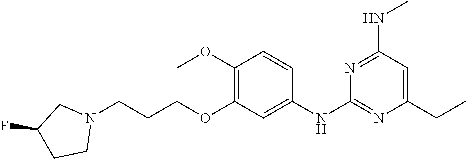 [SMILES: CCc1cc(NC)nc(Nc2ccc(OC)c(OCCCN3CC[C@@H](F)C3)c2)n1] |
| 386 | 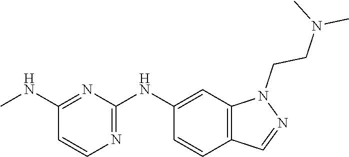 |
| 387 | 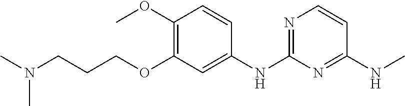 |
| 388 | 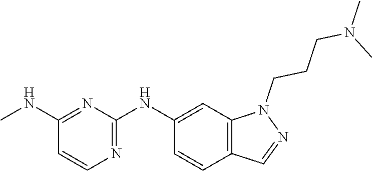 |
| 389 | 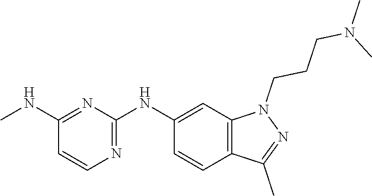 |
| 390 | 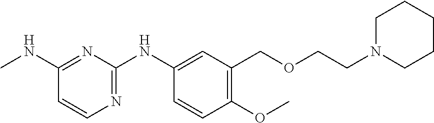 [SMILES: CNc1ccnc(Nc2ccc(OC)c(COCCN3CCCCC3)c2)n1] |
| 391 | 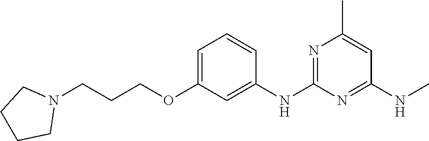 [SMILES: CNc1cc(C)nc(Nc2cccc(OCCCN3CCCC3)c2)n1] |
| 392 | 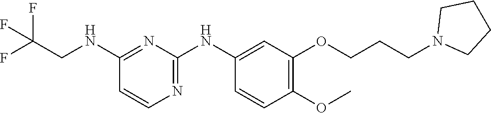 [SMILES: COc1ccc(Nc2nccc(NCC(F)(F)F)n2)cc1OCCCN1CCCC1] |
| 393 | 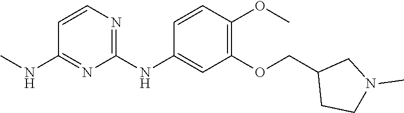 |
| 394 | 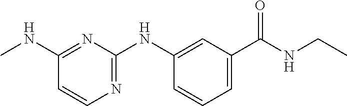 [SMILES: CCNC(=O)c1cccc(Nc2nccc(NC)n2)c1] |
| 395 | 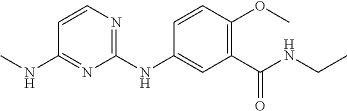 [SMILES: CCNC(=O)c1cc(Nc2nccc(NC)n2)ccc1OC] |
| 396 | 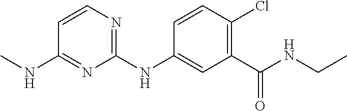 [SMILES: CCNC(=O)c1cc(Nc2nccc(NC)n2)ccc1Cl] |
| 397 | 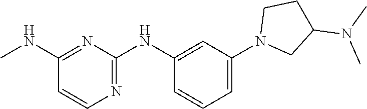 |
| 398 | 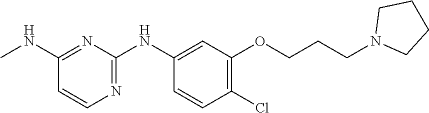 [SMILES: CNc1ccnc(Nc2ccc(Cl)c(OCCCN3CCCC3)c2)n1] |
| 399 | 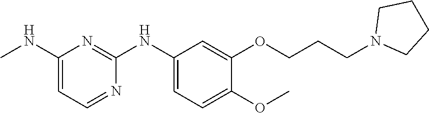 [SMILES: CNc1ccnc(Nc2ccc(OC)c(OCCCN3CCCC3)c2)n1] |
| 400 | 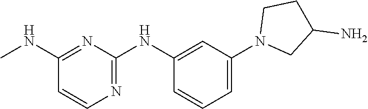 [SMILES: CNc1ccnc(Nc2cccc(N3CCC(N)C3)c2)n1] |
| 401 | 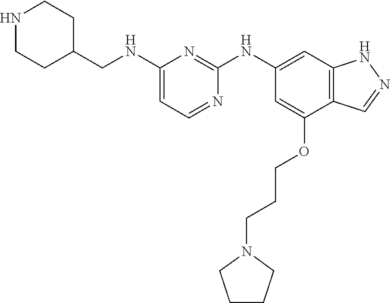 [SMILES: c1cc(NCC2CCNCC2)nc(Nc2cc(OCCCN3CCCC3)c3cn[nH]c3c2)n1] |
| 402 | 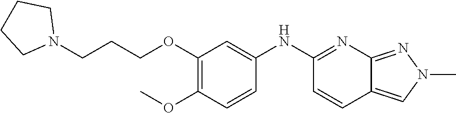 [SMILES: COc1ccc(Nc2ccc3cn(C)nc3n2)cc1OCCCN1CCCC1] |
| 404 | 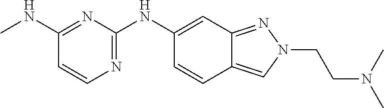 |
| 405 | 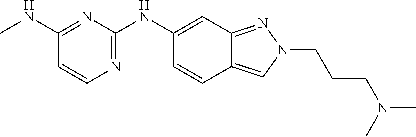 |
| 406 | 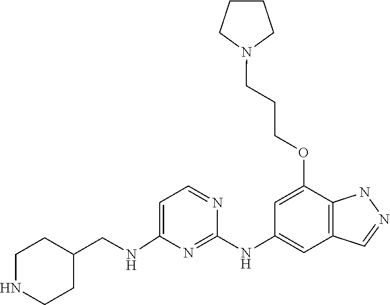 [SMILES: CN1CCC(CNc2ccnc(Nc3cc(OCCCN4CCCC4)c4[nH]ncc4c3)n2)CC1] |
| 407 | 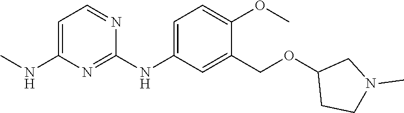 |
| 408 | 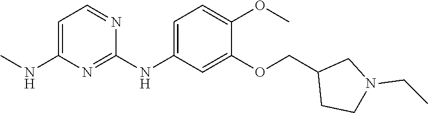 [SMILES: CCN1CCC(COc2cc(Nc3nccc(NC)n3)ccc2OC)C1] |
| 409 | 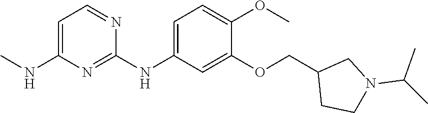 [SMILES: CNc1ccnc(Nc2ccc(OC)c(OCC3CCN(C(C)C)C3)c2)n1] |
| 410 | 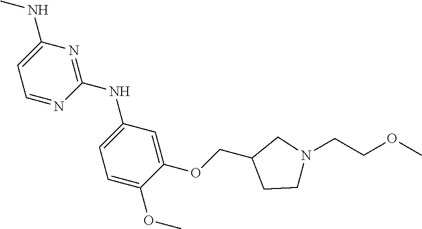 |
| 411 | 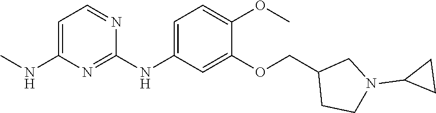 [SMILES: CNc1ccnc(Nc2ccc(OC)c(OCC3CCN(C4CC4)C3)c2)n1] |
| 412 | 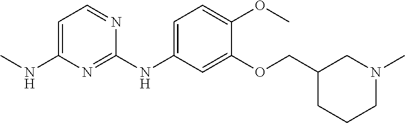 |
| 413 | 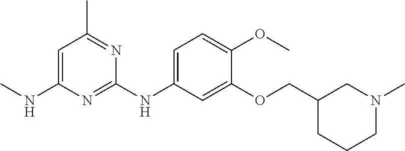 |
| 414 | 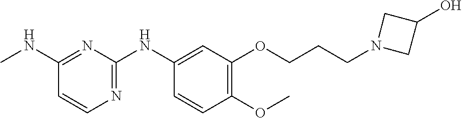 [SMILES: CNc1ccnc(Nc2ccc(OC)c(OCCCN3CC(O)C3)c2)n1] |
| 415 | 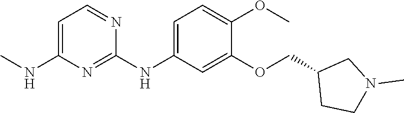 |
| 416 | 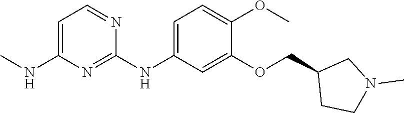 |
| 417 | 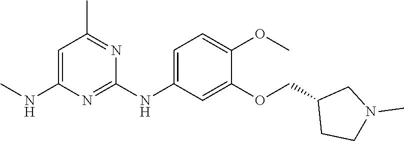 |
| 418 | 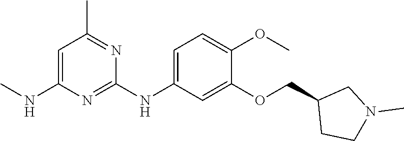 |
| 419 | 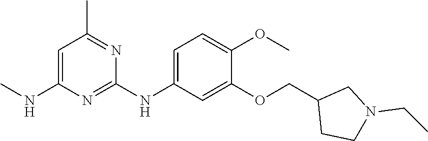 [SMILES: CCN1CCC(COc2cc(Nc3nc(C)cc(NC)n3)ccc2OC)C1] |
| 420 | 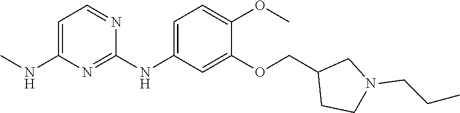 [SMILES: CCCN1CCC(COc2cc(Nc3nccc(NC)n3)ccc2OC)C1] |
| 421 | 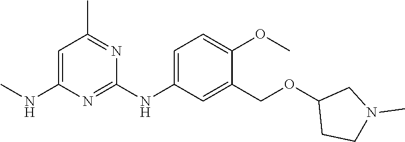 |
| 422 | 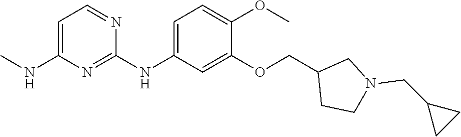 [SMILES: CNc1ccnc(Nc2ccc(OC)c(OCC3CCN(CC4CC4)C3)c2)n1] |
| 423 | 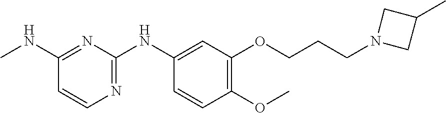 |
| 424 | 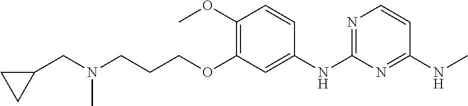 [SMILES: CNc1ccnc(Nc2ccc(OC)c(OCCCN(C)CC3CC3)c2)n1] |
| 425 | 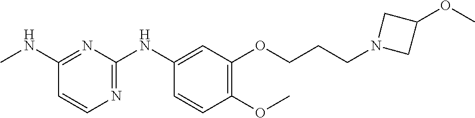 |
| 426 | 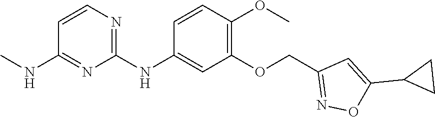 [SMILES: CNc1ccnc(Nc2ccc(OC)c(OCc3cc(C4CC4)on3)c2)n1] |
| 427 | 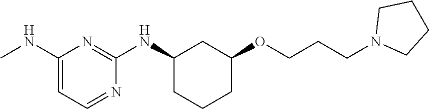 [SMILES: CNc1ccnc(N[C@@H]2CCC[C@H](OCCCN3CCCC3)C2)n1] |
| 428 | 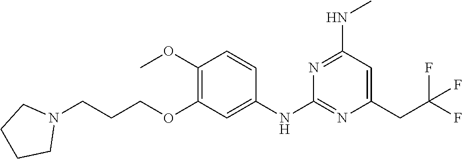 [SMILES: CNc1cc(CC(F)(F)F)nc(Nc2ccc(OC)c(OCCCN3CCCC3)c2)n1] |
| 429 | 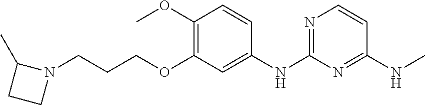 |
| 430 | 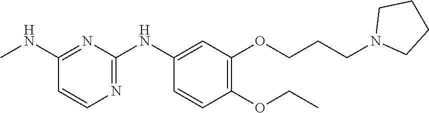 [SMILES: CCOc1ccc(Nc2nccc(NC)n2)cc1OCCCN1CCCC1] |
| 431 | 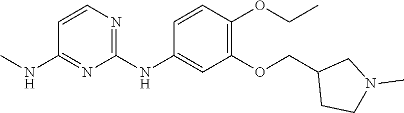 |
| 432 | 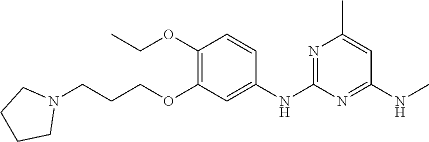 [SMILES: CCOc1ccc(Nc2nc(C)cc(NC)n2)cc1OCCCN1CCCC1] |
| 433 | 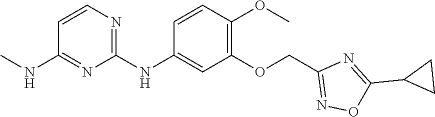 [SMILES: CNc1ccnc(Nc2ccc(OC)c(OCc3noc(C4CC4)n3)c2)n1] |
| 434 | 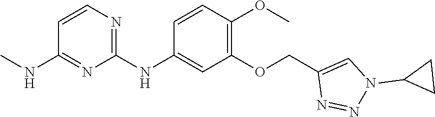 [SMILES: CNc1ccnc(Nc2ccc(OC)c(OCc3cn(C4CC4)nn3)c2)n1] |
| 435 | 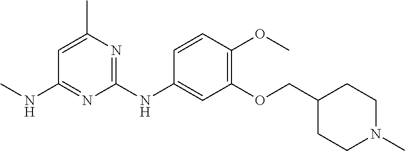 |
| 436 | 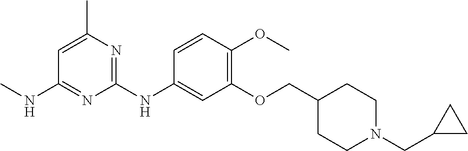 [SMILES: CNc1cc(C)nc(Nc2ccc(OC)c(OCC3CCN(CC4CC4)CC3)c2)n1] |
| 437 | 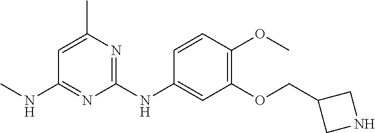 [SMILES: CNc1cc(C)nc(Nc2ccc(OC)c(OCC3CNC3)c2)n1] |
| 438 | 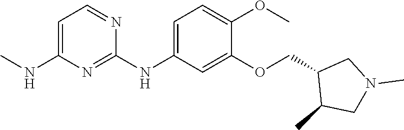 |
| 439 | 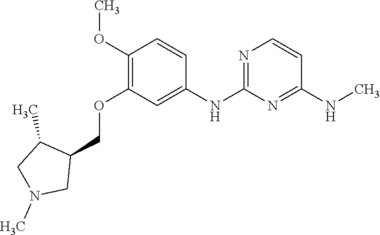 [SMILES: CNc1ccnc(Nc2ccc(OC)c(OC[C@H]3CN(C)C[C@@H]3C)c2)n1] |
| 440 | 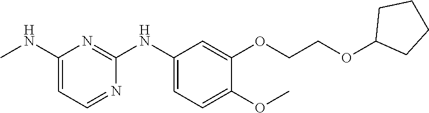 [SMILES: CNc1ccnc(Nc2ccc(OC)c(OCCOC3CCCC3)c2)n1] |
| 441 | 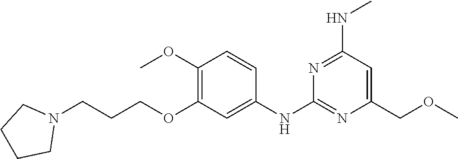 [SMILES: CNc1cc(COC)nc(Nc2ccc(OC)c(OCCCN3CCCC3)c2)n1] |
| 442 | 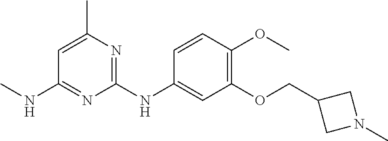 |
| 443 | 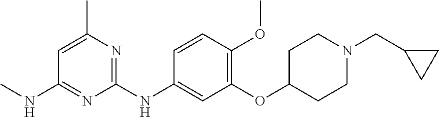 [SMILES: CNc1cc(C)nc(Nc2ccc(OC)c(OC3CCN(CC4CC4)CC3)c2)n1] |
| 444 | 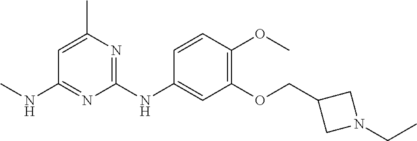 [SMILES: CCN1CC(COc2cc(Nc3nc(C)cc(NC)n3)ccc2OC)C1] |
| 445 | 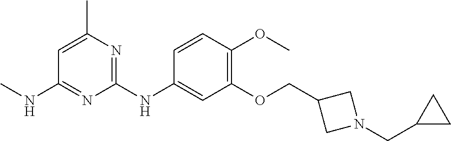 [SMILES: CNc1cc(C)nc(Nc2ccc(OC)c(OCC3CN(CC4CC4)C3)c2)n1] |
| 446 | 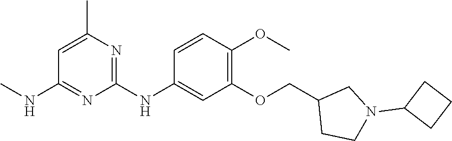 [SMILES: CNc1cc(C)nc(Nc2ccc(OC)c(OCC3CCN(C4CCC4)C3)c2)n1] |
| 447 | 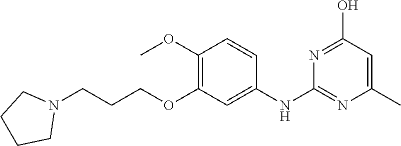 [SMILES: COc1ccc(Nc2nc(C)cc(O)n2)cc1OCCCN1CCCC1] |
| 448 | 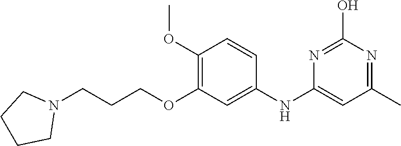 [SMILES: COc1ccc(Nc2cc(C)nc(O)n2)cc1OCCCN1CCCC1] |
| 449 | 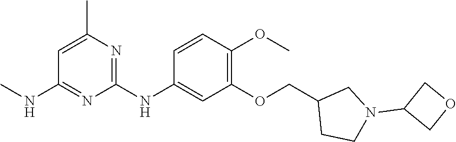 [SMILES: CNc1cc(C)nc(Nc2ccc(OC)c(OCC3CCN(C4COC4)C3)c2)n1] |
| 450 | 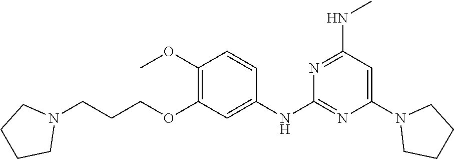 [SMILES: CNc1cc(N2CCCC2)nc(Nc2ccc(OC)c(OCCCN3CCCC3)c2)n1] |
| 451 | 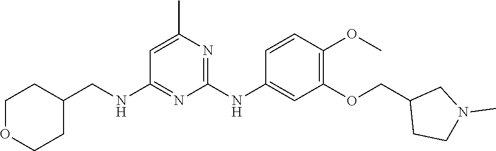 [SMILES: COc1ccc(Nc2nc(C)cc(NCC3CCOCC3)n2)cc1OCC1CCN(C)C1] |
| 452 | 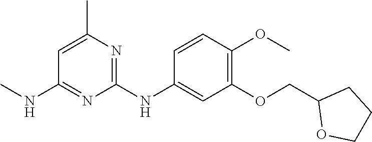 [SMILES: CNc1cc(C)nc(Nc2ccc(OC)c(OCC3CCCO3)c2)n1] |
| 453 | 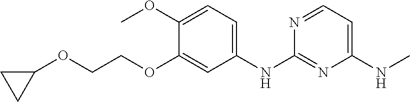 [SMILES: CNc1ccnc(Nc2ccc(OC)c(OCCOC3CC3)c2)n1] |
| 455 | 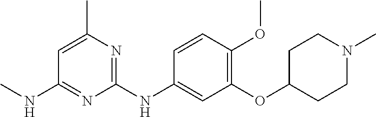 |
| 456 | 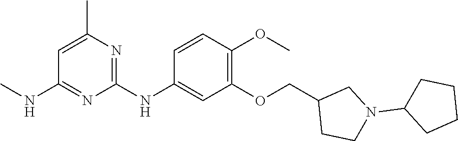 [SMILES: CNc1cc(C)nc(Nc2ccc(OC)c(OCC3CCN(C4CCCC4)C3)c2)n1] |
| 457 | 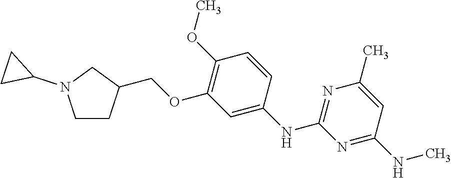 [SMILES: CNc1cc(C)nc(Nc2ccc(OC)c(OCC3CCN(C4CC4)C3)c2)n1] |
| 458 | 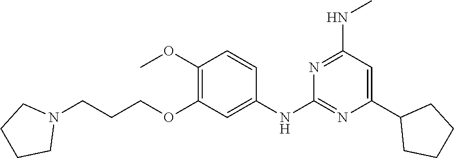 [SMILES: CNc1cc(C2CCCC2)nc(Nc2ccc(OC)c(OCCCN3CCCC3)c2)n1] |
| 459 | 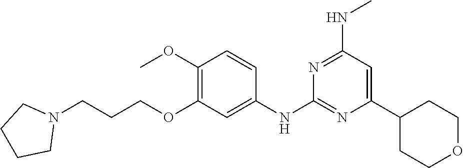 [SMILES: CNc1cc(C2CCOCC2)nc(Nc2ccc(OC)c(OCCCN3CCCC3)c2)n1] |
| 460 | 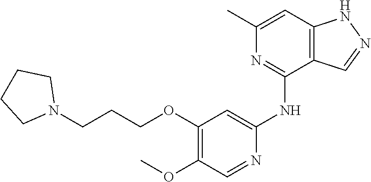 [SMILES: COc1cnc(Nc2nc(C)cc3[nH]ncc23)cc1OCCCN1CCCC1] |
| 461 | 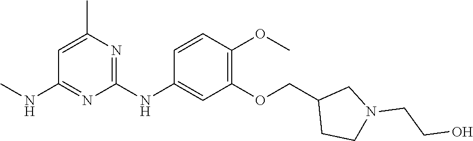 [SMILES: CNc1cc(C)nc(Nc2ccc(OC)c(OCC3CCN(CCO)C3)c2)n1] |
| 462 | 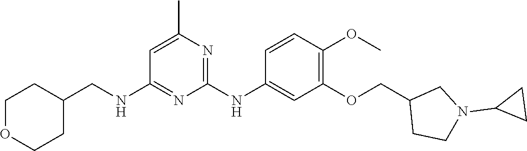 [SMILES: COc1ccc(Nc2nc(C)cc(NCC3CCOCC3)n2)cc1OCC1CCN(C2CC2)C1] |
| 463 | 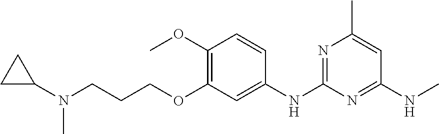 [SMILES: CNc1cc(C)nc(Nc2ccc(OC)c(OCCCN(C)C3CC3)c2)n1] |
| 464 | 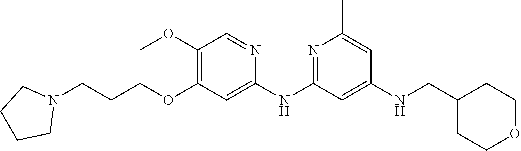 [SMILES: COc1cnc(Nc2cc(NCC3CCOCC3)cc(C)n2)cc1OCCCN1CCCC1] |
| 465 | 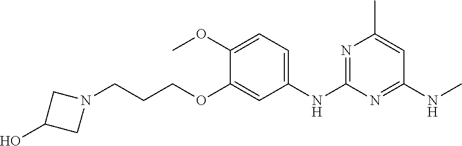 [SMILES: CNc1cc(C)nc(Nc2ccc(OC)c(OCCCN3CC(O)C3)c2)n1] |
| 466 | 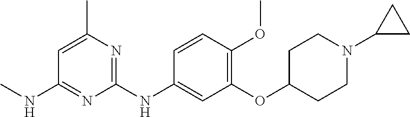 [SMILES: CNc1cc(C)nc(Nc2ccc(OC)c(OC3CCN(C4CC4)CC3)c2)n1] |
| 467 | 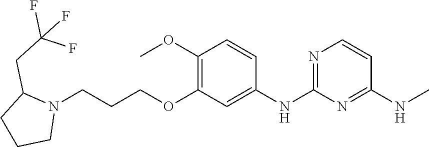 [SMILES: CNc1ccnc(Nc2ccc(OC)c(OCCCN3CCCC3CC(F)(F)F)c2)n1] |
| 468 | 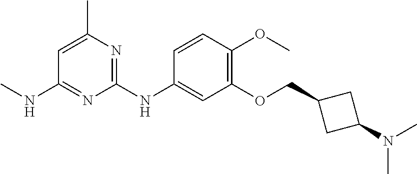 |
| 469 | 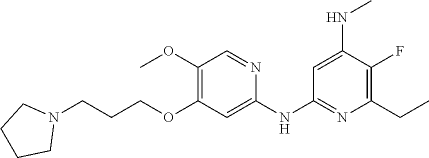 [SMILES: CCc1nc(Nc2cc(OCCCN3CCCC3)c(OC)cn2)cc(NC)c1F] |
| 470 | 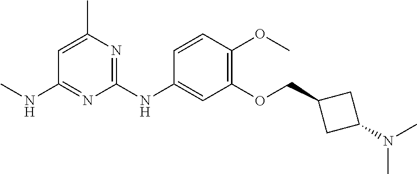 |
| 471 | 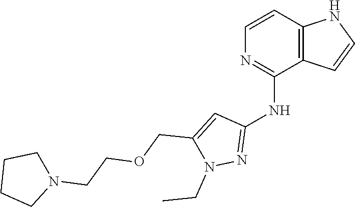 [SMILES: CCn1nc(Nc2nccc3[nH]ccc23)cc1COCCN1CCCC1] |
| 472 | 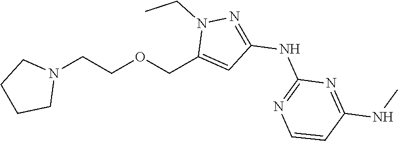 [SMILES: CCn1nc(Nc2nccc(NC)n2)cc1COCCN1CCCC1] |
| 473 | 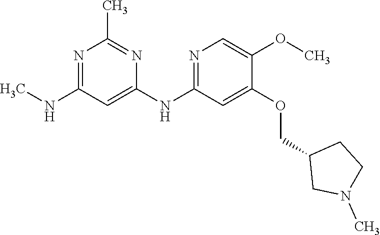 [SMILES: CNc1cc(Nc2cc(OC[C@@H]3CCN(C)C3)c(OC)cn2)nc(C)n1] |
| 474 | 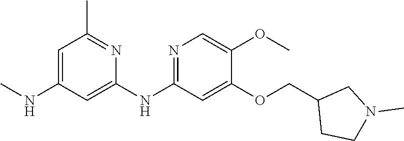 |
| 475 | 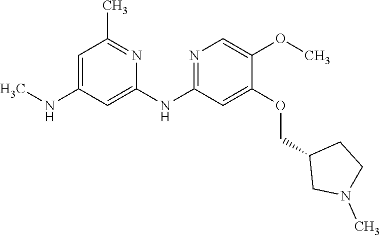 [SMILES: CNc1cc(C)nc(Nc2cc(OC[C@@H]3CCN(C)C3)c(OC)cn2)c1] |
| 476 | 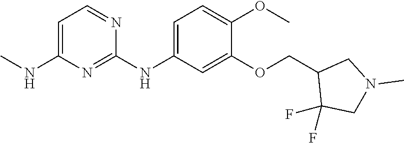 |
| 477 | 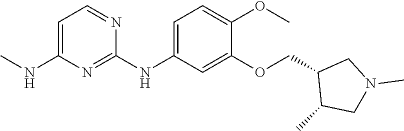 |
| 478 | 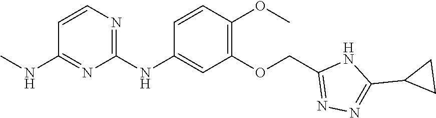 [SMILES: CNc1ccnc(Nc2ccc(OC)c(OCc3nnc(C4CC4)[nH]3)c2)n1] |
| 479 | 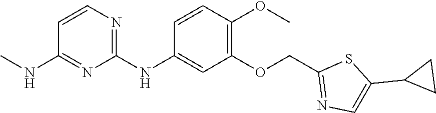 [SMILES: CNc1ccnc(Nc2ccc(OC)c(OCc3ncc(C4CC4)s3)c2)n1] |
| 480 | 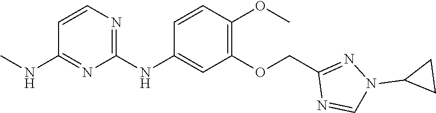 [SMILES: CNc1ccnc(Nc2ccc(OC)c(OCc3ncn(C4CC4)n3)c2)n1] |
| 481 | 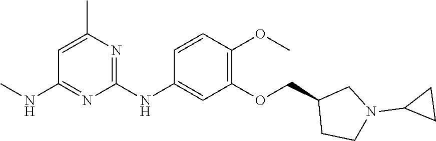 [SMILES: CNc1cc(C)nc(Nc2ccc(OC)c(OC[C@@H]3CCN(C4CC4)C3)c2)n1] |
| 482 | 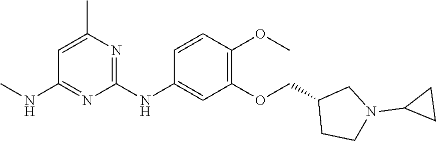 [SMILES: CNc1cc(C)nc(Nc2ccc(OC)c(OC[C@H]3CCN(C4CC4)C3)c2)n1] |
| 483 | 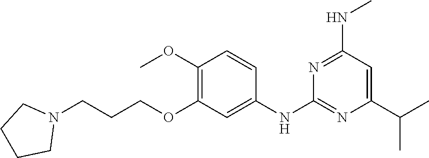 [SMILES: CNc1cc(C(C)C)nc(Nc2ccc(OC)c(OCCCN3CCCC3)c2)n1] |
| 484 | 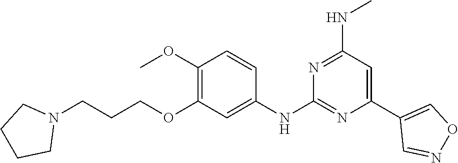 [SMILES: CNc1cc(-c2cnoc2)nc(Nc2ccc(OC)c(OCCCN3CCCC3)c2)n1] |
| 485 | 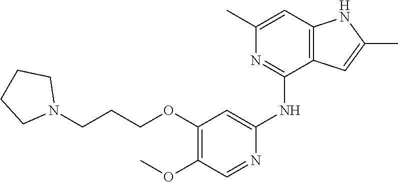 [SMILES: COc1cnc(Nc2nc(C)cc3[nH]c(C)cc23)cc1OCCCN1CCCC1] |
| 486 | 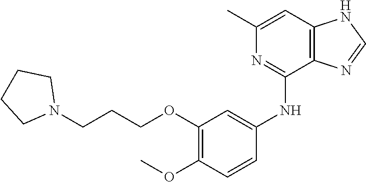 [SMILES: COc1ccc(Nc2nc(C)cc3[nH]cnc23)cc1OCCCN1CCCC1] |
| 487 | 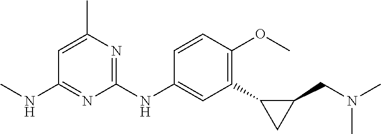 |
| 488 | 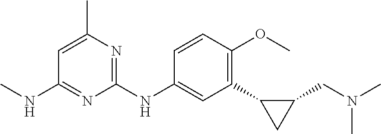 |
| 489 | 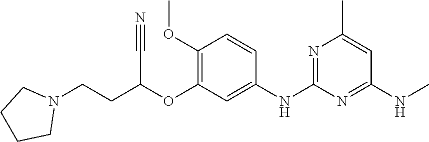 [SMILES: CNc1cc(C)nc(Nc2ccc(OC)c(OC(C#N)CCN3CCCC3)c2)n1] |
| 490 | 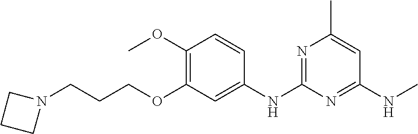 [SMILES: CNc1cc(C)nc(Nc2ccc(OC)c(OCCCN3CCC3)c2)n1] |
| 491 | 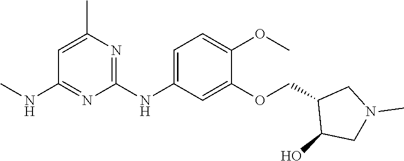 |
| 492 | 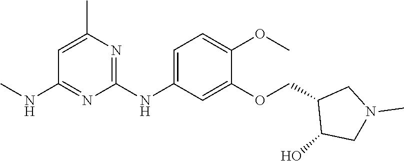 |
| 493 | 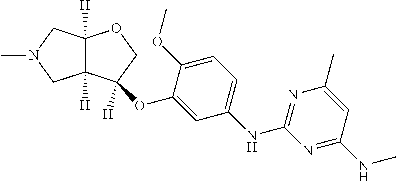 |
| 494 | 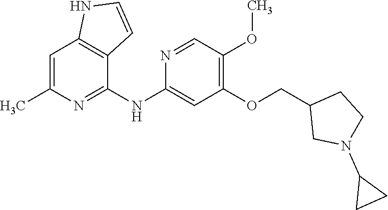 [SMILES: COc1cnc(Nc2nc(C)cc3[nH]ccc23)cc1OCC1CCN(C2CC2)C1] |
| 494a | 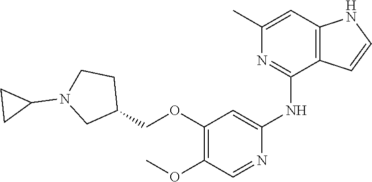 [SMILES: COc1cnc(Nc2nc(C)cc3[nH]ccc23)cc1OC[C@H]1CCN(C2CC2)C1] |
| 495 | 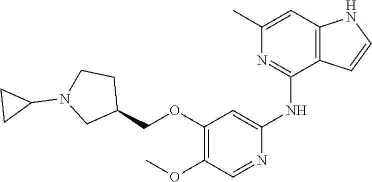 [SMILES: COc1cnc(Nc2nc(C)cc3[nH]ccc23)cc1OC[C@@H]1CCN(C2CC2)C1] |
| 496 | 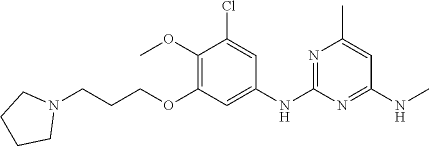 [SMILES: CNc1cc(C)nc(Nc2cc(Cl)c(OC)c(OCCCN3CCCC3)c2)n1] |
| 497 | 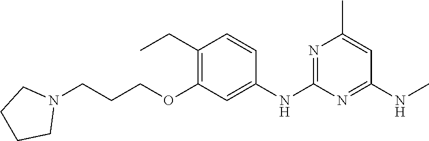 [SMILES: CCc1ccc(Nc2nc(C)cc(NC)n2)cc1OCCCN1CCCC1] |
| 498 | 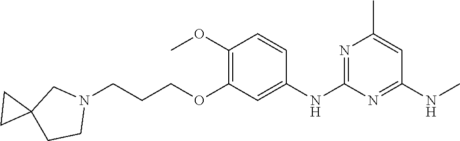 [SMILES: CNc1cc(C)nc(Nc2ccc(OC)c(OCCCN3CCC4(CC4)C3)c2)n1] |
| 499 | 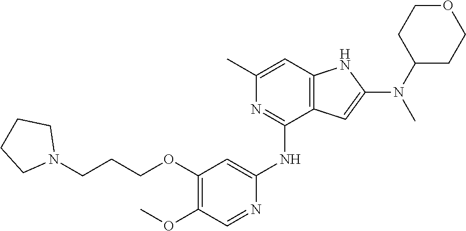 [SMILES: COc1cnc(Nc2nc(C)cc3[nH]c(N(C)C4CCOCC4)cc23)cc1OCCCN1CCCC1] |
| 500 | 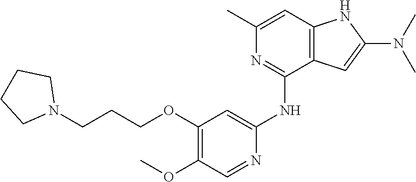 [SMILES: COc1cnc(Nc2nc(C)cc3[nH]c(N(C)C)cc23)cc1OCCCN1CCCC1] |
| 501 | 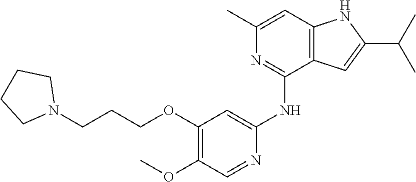 [SMILES: COc1cnc(Nc2nc(C)cc3[nH]c(C(C)C)cc23)cc1OCCCN1CCCC1] |
| 502 | 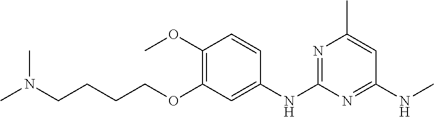 |
| 503 | 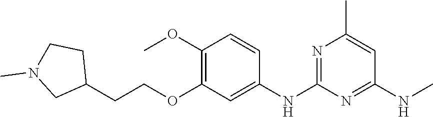 |
| 504 | 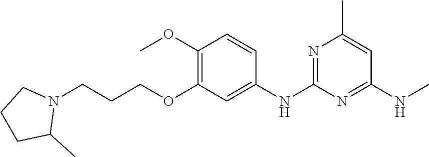 [SMILES: CNc1cc(C)nc(Nc2ccc(OC)c(OCCCN3CCCC3C)c2)n1] |
| 505 | 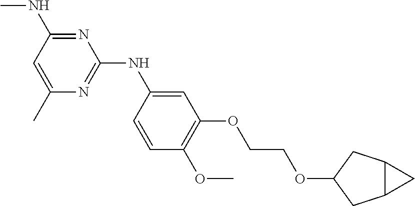 [SMILES: CNc1cc(C)nc(Nc2ccc(OC)c(OCCOC3CC4CC4C3)c2)n1] |
| 506 | 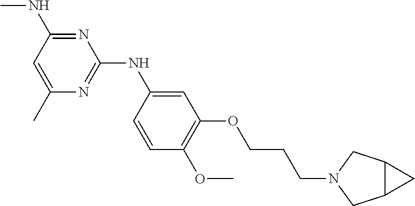 [SMILES: CNc1cc(C)nc(Nc2ccc(OC)c(OCCCN3CC4CC4C3)c2)n1] |
| 507 | 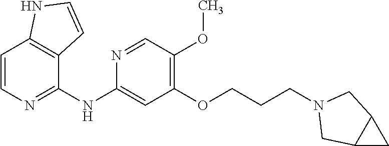 [SMILES: COc1cnc(Nc2nccc3[nH]ccc23)cc1OCCCN1CC2CC2C1] |
| 508 | 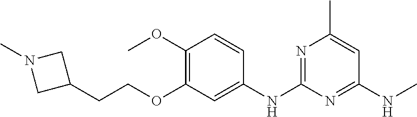 |
| 509 | 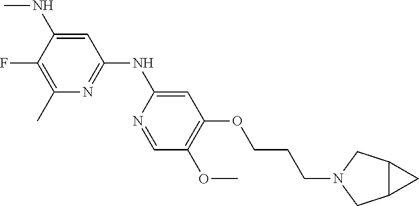 [SMILES: CNc1cc(Nc2cc(OCCCN3CC4CC4C3)c(OC)cn2)nc(C)c1F] |
| 510 | 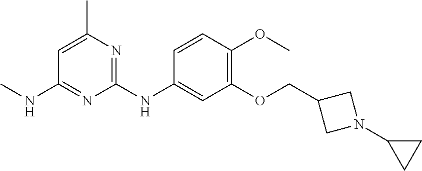 [SMILES: CNc1cc(C)nc(Nc2ccc(OC)c(OCC3CN(C4CC4)C3)c2)n1] |
| 511 | 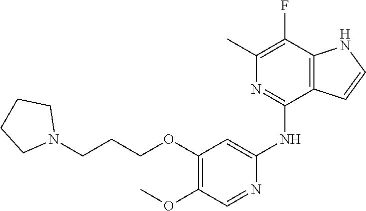 [SMILES: COc1cnc(Nc2nc(C)c(F)c3[nH]ccc23)cc1OCCCN1CCCC1] |
| 512 | 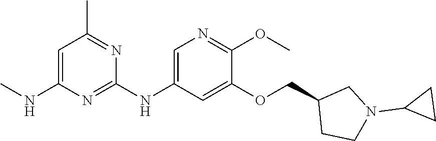 [SMILES: CNc1cc(C)nc(Nc2cnc(OC)c(OC[C@@H]3CCN(C4CC4)C3)c2)n1] |
| 513 | 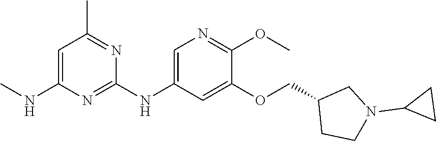 [SMILES: CNc1cc(C)nc(Nc2cnc(OC)c(OC[C@H]3CCN(C4CC4)C3)c2)n1] |
| 514 | 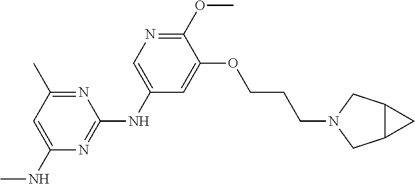 [SMILES: CNc1cc(C)nc(Nc2cnc(OC)c(OCCCN3CC4CC4C3)c2)n1] |
| 515 | 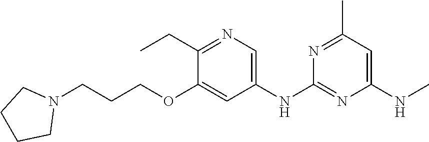 [SMILES: CCc1ncc(Nc2nc(C)cc(NC)n2)cc1OCCCN1CCCC1] |
| 516 | 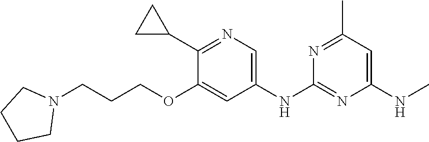 [SMILES: CNc1cc(C)nc(Nc2cnc(C3CC3)c(OCCCN3CCCC3)c2)n1] |
| 517a | 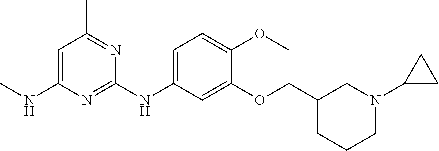 [SMILES: CNc1cc(C)nc(Nc2ccc(OC)c(OCC3CCCN(C4CC4)C3)c2)n1] |
| 517b | 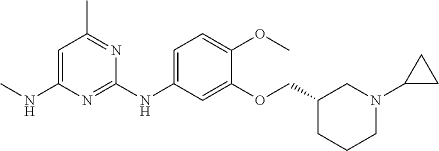 [SMILES: CNc1cc(C)nc(Nc2ccc(OC)c(OC[C@H]3CCCN(C4CC4)C3)c2)n1] |

TABLE 1C

| Comd. | |
|---|---|
| No. | Structure |
| 270 | 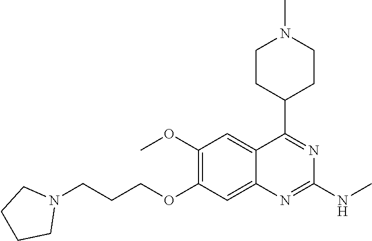 [SMILES: CNc1nc(C2CCN(C)CC2)c2cc(OC)c(OCCCN3CCCC3)cc2n1] |
| 518 | 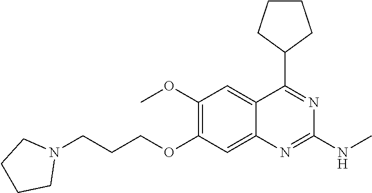 [SMILES: CNc1nc(C2CCCC2)c2cc(OC)c(OCCCN3CCCC3)cc2n1] |
| 519 | 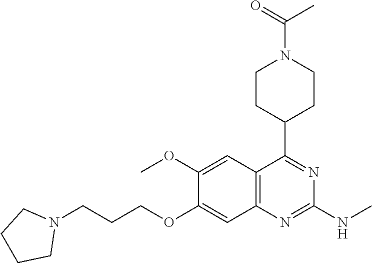 [SMILES: CNc1nc(C2CCN(C(C)=O)CC2)c2cc(OC)c(OCCCN3CCCC3)cc2n1] |
| 520 | 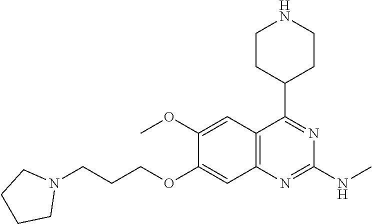 [SMILES: CNc1nc(C2CCNCC2)c2cc(OC)c(OCCCN3CCCC3)cc2n1] |
| 521 | 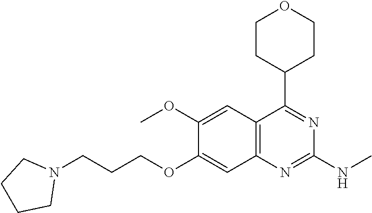 [SMILES: CNc1nc(C2CCOCC2)c2cc(OC)c(OCCCN3CCCC3)cc2n1] |
| 522 | 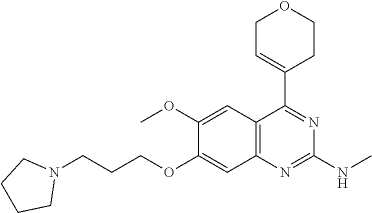 [SMILES: CNc1nc(C2=CCOCC2)c2cc(OC)c(OCCCN3CCCC3)cc2n1] |
| 523 | 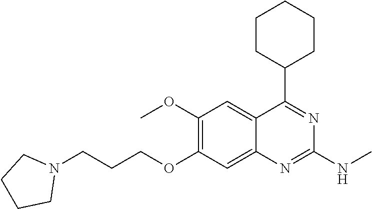 [SMILES: CNc1nc(C2CCCCC2)c2cc(OC)c(OCCCN3CCCC3)cc2n1] |
| 524 | 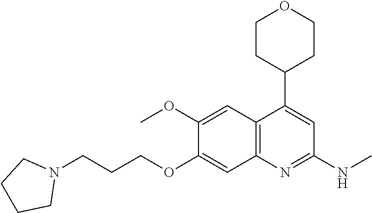 [SMILES: CNc1cc(C2CCOCC2)c2cc(OC)c(OCCCN3CCCC3)cc2n1] |
| 525 | 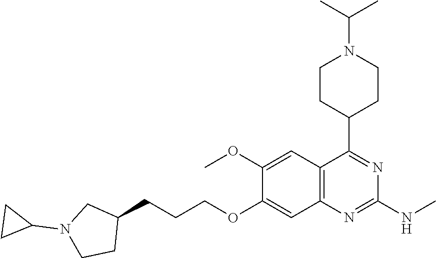 [SMILES: CNc1nc(C2CCN(C(C)C)CC2)c2cc(OC)c(OCCC[C@H]3CCN(C4CC4)C3)cc2n1] |
| 526 | 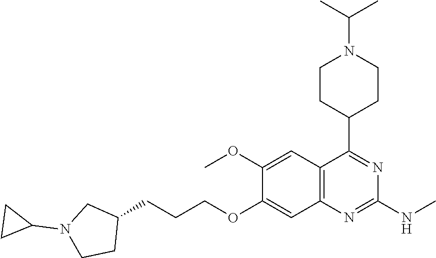 [SMILES: CNc1nc(C2CCN(C(C)C)CC2)c2cc(OC)c(OCCC[C@@H]3CCN(C4CC4)C3)cc2n1] |
| 527 | 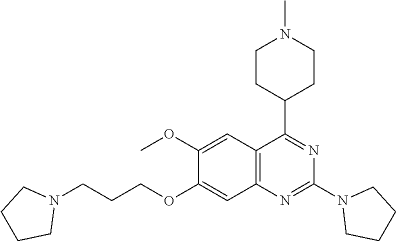 [SMILES: COc1cc2c(C3CCN(C)CC3)nc(N3CCCC3)nc2cc1OCCCN1CCCC1] |
| 528 | 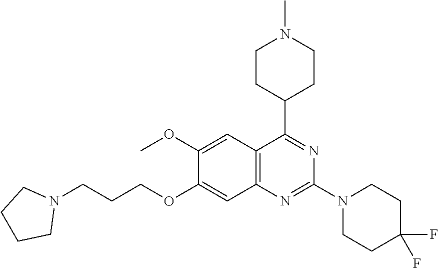 [SMILES: COc1cc2c(C3CCN(C)CC3)nc(N3CCC(F)(F)CC3)nc2cc1OCCCN1CCCC1] |
| 529 | 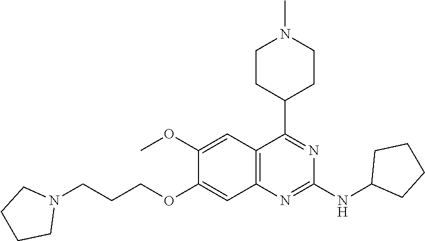 [SMILES: COc1cc2c(C3CCN(C)CC3)nc(NC3CCCC3)nc2cc1OCCCN1CCCC1] |
| 530 | 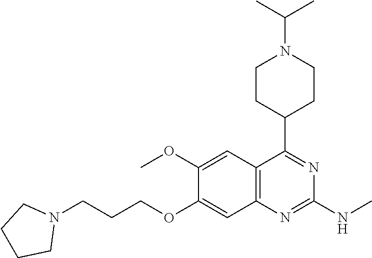 [SMILES: CNc1nc(C2CCN(C(C)C)CC2)c2cc(OC)c(OCCCN3CCCC3)cc2n1] |
| 531 | 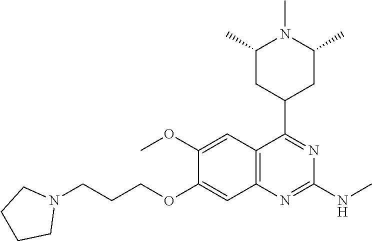 [SMILES: CNc1nc(C2C[C@@H](C)N(C)[C@@H](C)C2)c2cc(OC)c(OCCCN3CCCC3)cc2n1] |
| 532 | 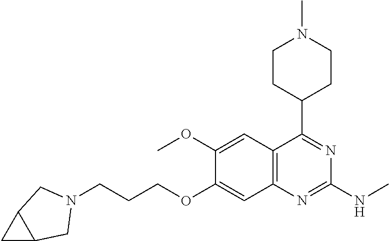 [SMILES: CNc1nc(C2CCN(C)CC2)c2cc(OC)c(OCCCN3CC4CC4C3)cc2n1] |
| 533 | 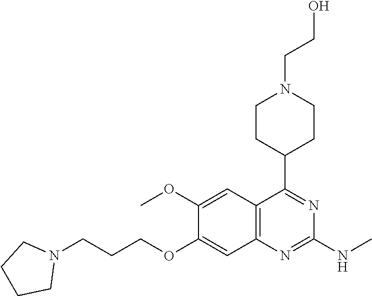 [SMILES: CNc1nc(C2CCN(CCO)CC2)c2cc(OC)c(OCCCN3CCCC3)cc2n1] |
| 534 | 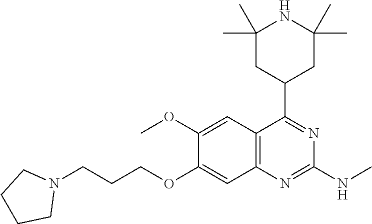 [SMILES: CNc1nc(C2CC(C)(C)NC(C)(C)C2)c2cc(OC)c(OCCCN3CCCC3)cc2n1] |
| 535 | 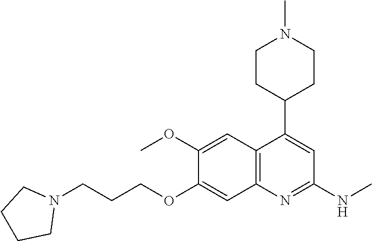 [SMILES: CNc1cc(C2CCN(C)CC2)c2cc(OC)c(OCCCN3CCCC3)cc2n1] |
| 536 | 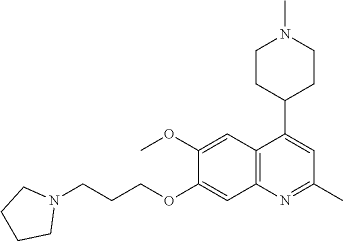 [SMILES: COc1cc2c(C3CCN(C)CC3)cc(C)nc2cc1OCCCN1CCCC1] |
| 537 | 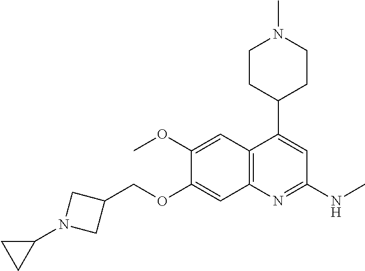 [SMILES: CNc1cc(C2CCN(C)CC2)c2cc(OC)c(OCC3CN(C4CC4)C3)cc2n1] |
| 538 | 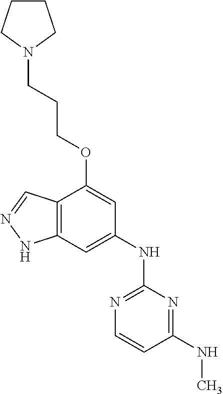 [SMILES: CNc1ccnc(Nc2cc(OCCCN3CCCC3)c3cn[nH]c3c2)n1] |
| 539 | 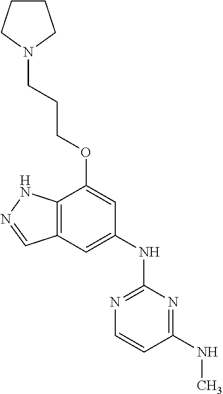 [SMILES: CNc1ccnc(Nc2cc(OCCCN3CCCC3)c3[nH]ncc3c2)n1] |
| 540 | 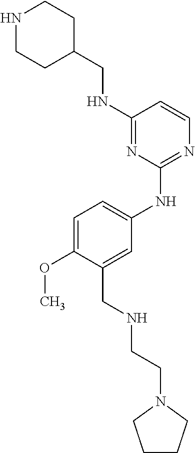 [SMILES: COc1ccc(Nc2nccc(NCC3CCNCC3)n2)cc1CNCCN1CCCC1] |
| 541 | 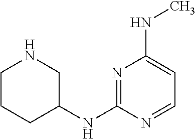 |
| 542 | 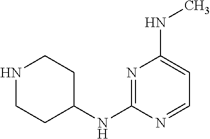 |
| 543 | 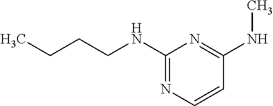 |
| 544 | 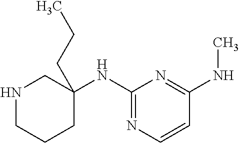 [SMILES: CCCC1(Nc2nccc(NC)n2)CCCNC1] |
| 545 | 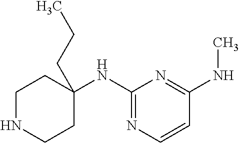 [SMILES: CCCC1(Nc2nccc(NC)n2)CCNCC1] |
| 546 | 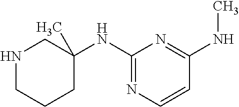 [SMILES: CNc1ccnc(NC2(C)CCCNC2)n1] |
| 547 | 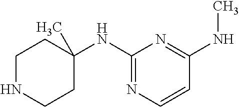 [SMILES: CNc1ccnc(NC2(C)CCNCC2)n1] |
| 548 | 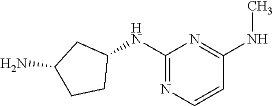 |
| 549 | 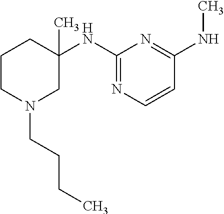 [SMILES: CCCCN1CCCC(C)(Nc2nccc(NC)n2)C1] |
| 550 | 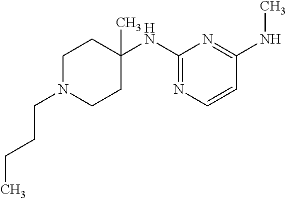 [SMILES: CCCCN1CCC(C)(Nc2nccc(NC)n2)CC1] |
| 551 | 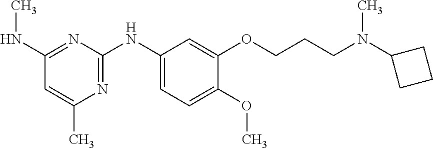 [SMILES: CNc1cc(C)nc(Nc2ccc(OC)c(OCCCN(C)C3CCC3)c2)n1] |
| 552 | 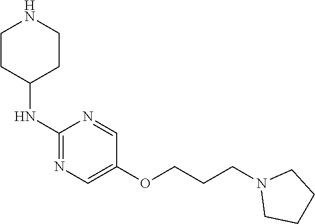 [SMILES: c1nc(NC2CCNCC2)ncc1OCCCN1CCCC1] |
| 553 | 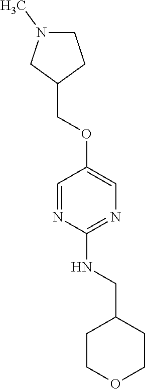 [SMILES: CN1CCC(COc2cnc(NCC3CCOCC3)nc2)C1] |
| 554 | 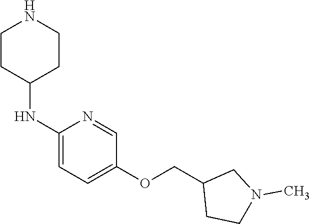 [SMILES: CN1CCC(COc2ccc(NC3CCNCC3)nc2)C1] |
| 555 | 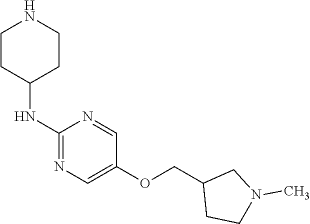 [SMILES: CN1CCC(COc2cnc(NC3CCNCC3)nc2)C1] |
| 556 | 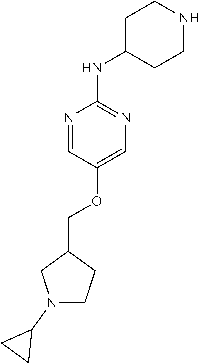 [SMILES: c1nc(NC2CCNCC2)ncc1OCC1CCN(C2CC2)C1] |
| 557 | 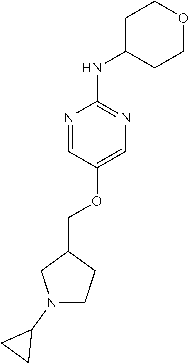 [SMILES: c1nc(NC2CCOCC2)ncc1OCC1CCN(C2CC2)C1] |
| 558 | 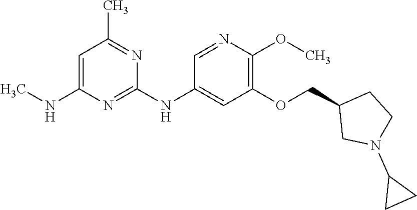 [SMILES: CNc1cc(C)nc(Nc2cnc(OC)c(OC[C@H]3CCN(C4CC4)C3)c2)n1] |
| 559 | 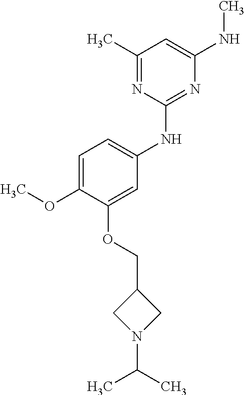 [SMILES: CNc1cc(C)nc(Nc2ccc(OC)c(OCC3CN(C(C)C)C3)c2)n1] |
| 560 | 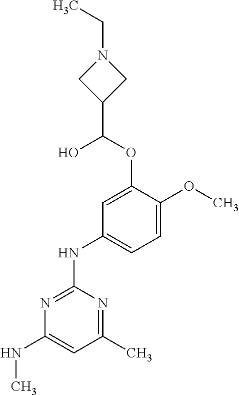 |
| 561 | 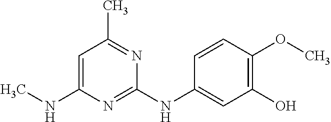 |
| 562 | 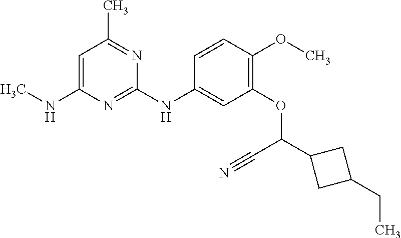 [SMILES: CCC1CC(C(C#N)Oc2cc(Nc3nc(C)cc(NC)n3)ccc2OC)C1] |
| 563 | 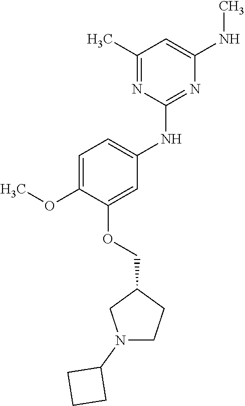 |
| 564 | 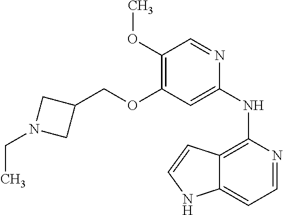 [SMILES: CCN1CC(COc2cc(Nc3nccc4[nH]ccc34)ncc2OC)C1] |
| 565 | 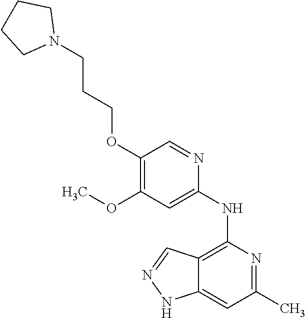 [SMILES: COc1cc(Nc2nc(C)cc3[nH]ncc23)ncc1OCCCN1CCCC1] |
| 566 | 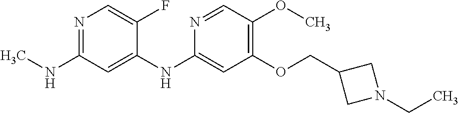 [SMILES: CCN1CC(COc2cc(Nc3cc(NC)ncc3F)ncc2OC)C1] |
| 567 | 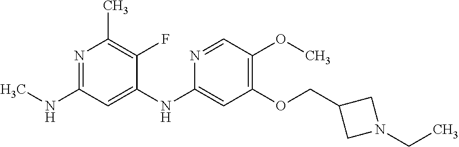 [SMILES: CCN1CC(COc2cc(Nc3cc(NC)nc(C)c3F)ncc2OC)C1] |
| 568 | 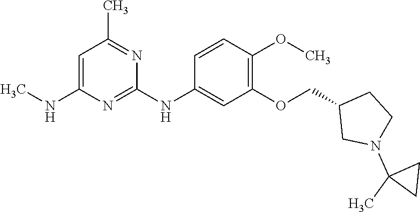 [SMILES: CNc1cc(C)nc(Nc2ccc(OC)c(OC[C@@H]3CCN(C4(C)CC4)C3)c2)n1] |
| 569 | 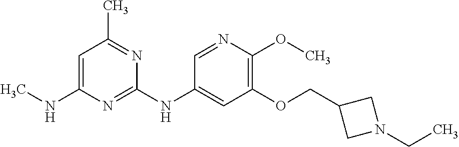 [SMILES: CCN1CC(COc2cc(Nc3nc(C)cc(NC)n3)cnc2OC)C1] |
| 570 | 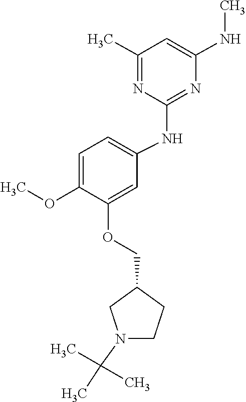 [SMILES: CNc1cc(C)nc(Nc2ccc(OC)c(OC[C@@H]3CCN(C(C)(C)C)C3)c2)n1] |
| 571 | 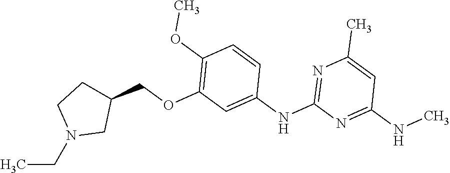 [SMILES: CCN1CC[C@@H](COc2cc(Nc3nc(C)cc(NC)n3)ccc2OC)C1] |
| 572 | 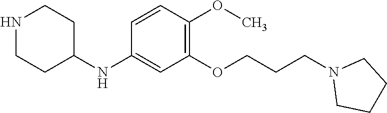 [SMILES: COc1ccc(NC2CCNCC2)cc1OCCCN1CCCC1] |
| 573 | 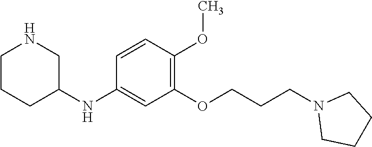 [SMILES: COc1ccc(NC2CCCNC2)cc1OCCCN1CCCC1] |
| 574 | 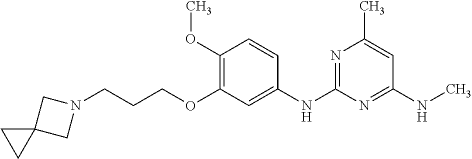 [SMILES: CNc1cc(C)nc(Nc2ccc(OC)c(OCCCN3CC4(CC4)C3)c2)n1] |
| 575 | 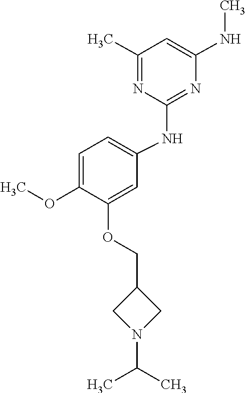 [SMILES: CNc1cc(C)nc(Nc2ccc(OC)c(OCC3CN(C(C)C)C3)c2)n1] |
| 576 | 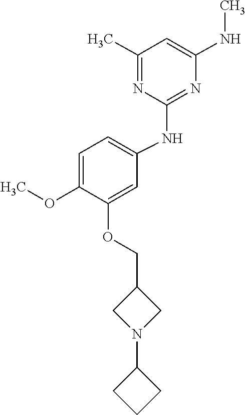 [SMILES: CNc1cc(C)nc(Nc2ccc(OC)c(OCC3CN(C4CCC4)C3)c2)n1] |
| 577 | 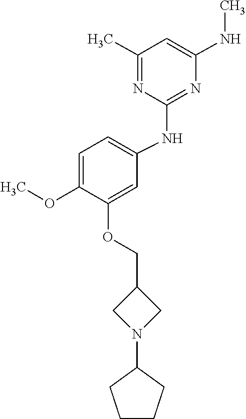 [SMILES: CNc1cc(C)nc(Nc2ccc(OC)c(OCC3CN(C4CCCC4)C3)c2)n1] |
| 578 | 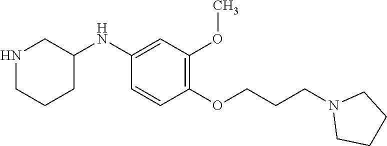 [SMILES: COc1cc(NC2CCCNC2)ccc1OCCCN1CCCC1] |
| 579 | 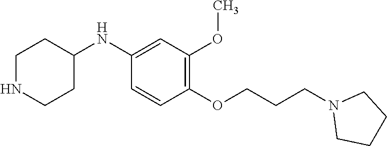 [SMILES: COc1cc(NC2CCNCC2)ccc1OCCCN1CCCC1] |
| 580 | 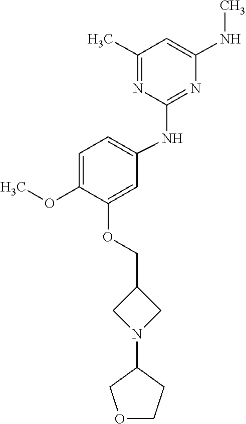 [SMILES: CNc1cc(C)nc(Nc2ccc(OC)c(OCC3CN(C4CCOC4)C3)c2)n1] |
| 581 | 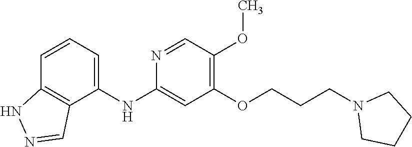 [SMILES: COc1cnc(Nc2cccc3[nH]ncc23)cc1OCCCN1CCCC1] |
| 582 | 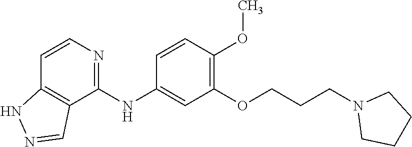 [SMILES: COc1ccc(Nc2nccc3[nH]ncc23)cc1OCCCN1CCCC1] |
| 583 | 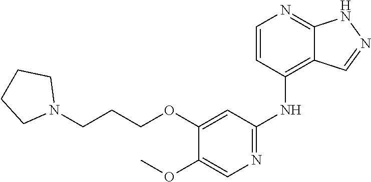 [SMILES: COc1cnc(Nc2ccnc3[nH]ncc23)cc1OCCCN1CCCC1] |
| 584 | 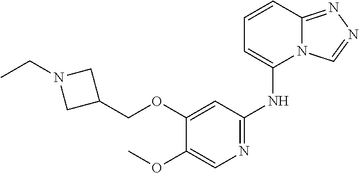 [SMILES: CCN1CC(COc2cc(Nc3cccc4nncn34)ncc2OC)C1] |
| 585 | 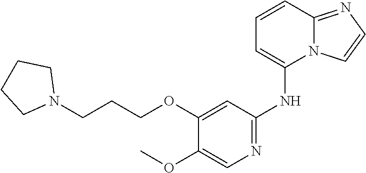 [SMILES: COc1cnc(Nc2cccc3nccn23)cc1OCCCN1CCCC1] |
| 586 | 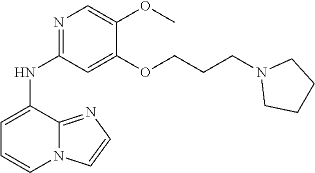 [SMILES: COc1cnc(Nc2cccn3ccnc23)cc1OCCCN1CCCC1] |
| 587 | 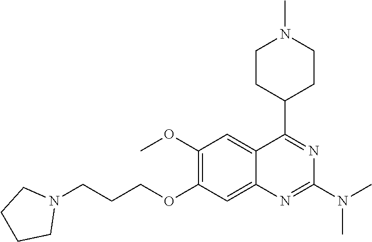 [SMILES: COc1cc2c(C3CCN(C)CC3)nc(N(C)C)nc2cc1OCCCN1CCCC1] |
| 588 | 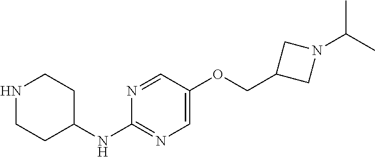 [SMILES: CC(C)N1CC(COc2cnc(NC3CCNCC3)nc2)C1] |
| 589 | 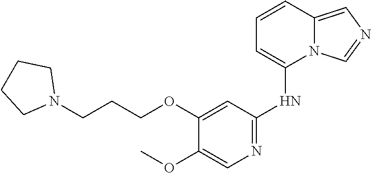 [SMILES: COc1cnc(Nc2cccc3cncn23)cc1OCCCN1CCCC1] |
| 590 | 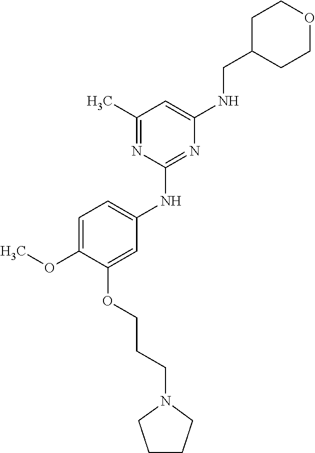 [SMILES: COc1ccc(Nc2nc(C)cc(NCC3CCOCC3)n2)cc1OCCCN1CCCC1] |
| 591 | 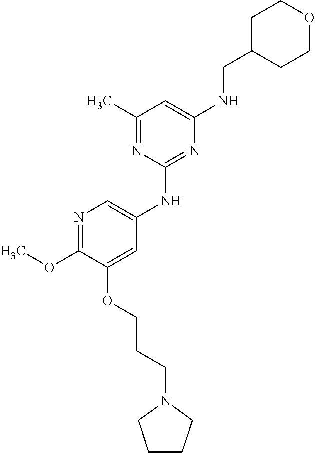 [SMILES: COc1ncc(Nc2nc(C)cc(NCC3CCOCC3)n2)cc1OCCCN1CCCC1] |
| 592 | 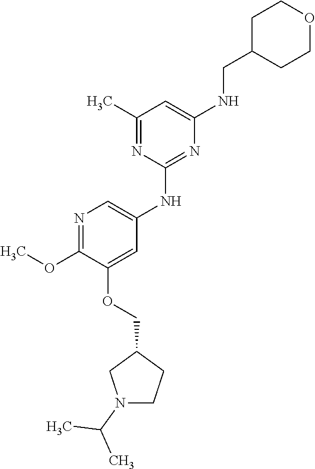 [SMILES: COc1ncc(Nc2nc(C)cc(NCC3CCOCC3)n2)cc1OC[C@@H]1CCN(C(C)C)C1] |
| 593 | 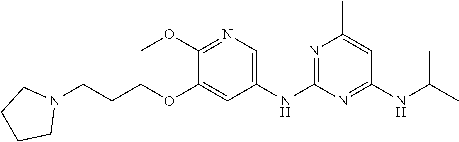 [SMILES: COc1ncc(Nc2nc(C)cc(NC(C)C)n2)cc1OCCCN1CCCC1] |
| 594 | 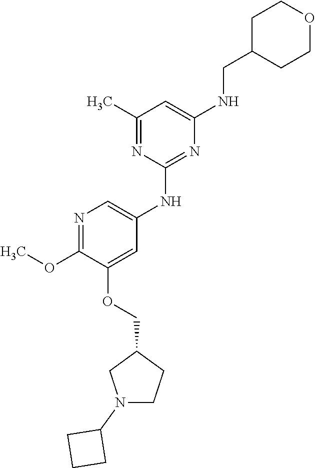 [SMILES: COc1ncc(Nc2nc(C)cc(NCC3CCOCC3)n2)cc1OC[C@@H]1CCN(C2CCC2)C1] |
| 595 | 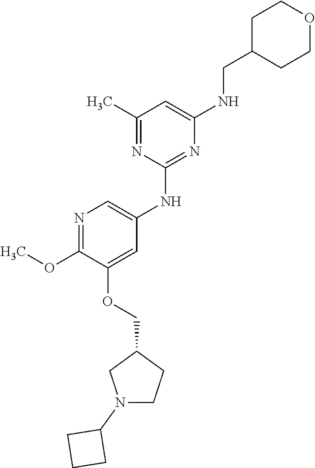 [SMILES: COc1ncc(Nc2nc(C)cc(NCC3CCOCC3)n2)cc1OC[C@@H]1CCN(C2CCC2)C1] |
| 596 | 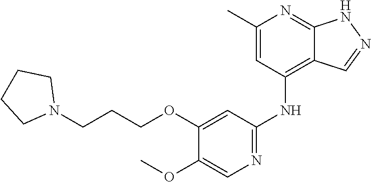 [SMILES: COc1cnc(Nc2cc(C)nc3[nH]ncc23)cc1OCCCN1CCCC1] |
| 597 | 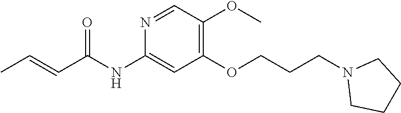 [SMILES: C/C=C/C(=O)Nc1cc(OCCCN2CCCC2)c(OC)cn1] |
| 598 | 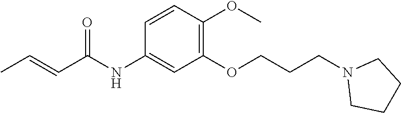 [SMILES: C/C=C/C(=O)Nc1ccc(OC)c(OCCCN2CCCC2)c1] |
| 599 | 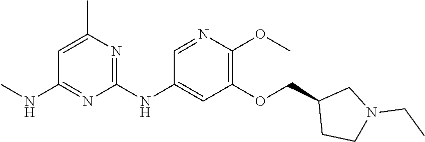 [SMILES: CCN1CC[C@@H](COc2cc(Nc3nc(C)cc(NC)n3)cnc2OC)C1] |
| 600 | 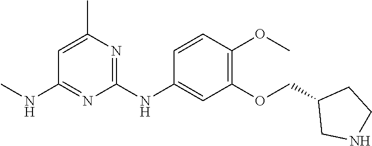 [SMILES: CNc1cc(C)nc(Nc2ccc(OC)c(OC[C@@H]3CCNC3)c2)n1] |
| 601 | 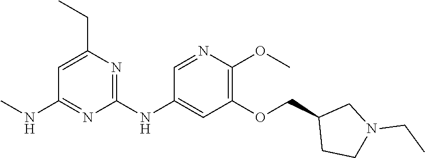 [SMILES: CCc1cc(NC)nc(Nc2cnc(OC)c(OC[C@@H]3CCN(CC)C3)c2)n1] |
| 602 | 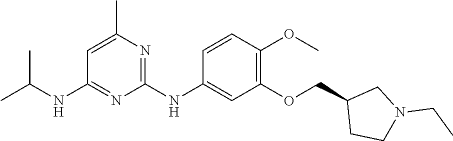 [SMILES: CCN1CC[C@@H](COc2cc(Nc3nc(C)cc(NC(C)C)n3)ccc2OC)C1] |
| 603 | 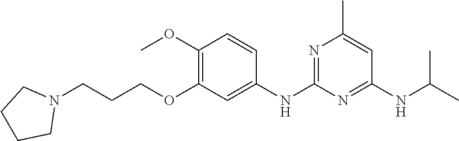 [SMILES: COc1ccc(Nc2nc(C)cc(NC(C)C)n2)cc1OCCCN1CCCC1] |
| 604 | 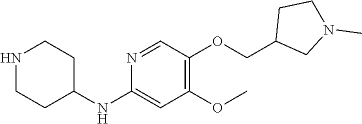 [SMILES: COc1cc(NC2CCNCC2)ncc1OCC1CCN(C)C1] |
| 605 | 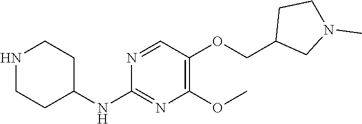 [SMILES: COc1nc(NC2CCNCC2)ncc1OCC1CCN(C)C1] |
| 606 | 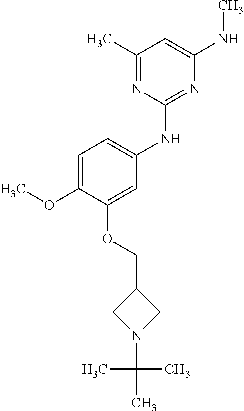 |
| 607 | 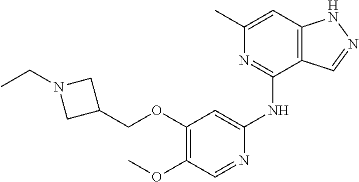 [SMILES: CCN1CC(COc2cc(Nc3nc(C)cc4[nH]ncc34)ncc2OC)C1] |
| 608 | 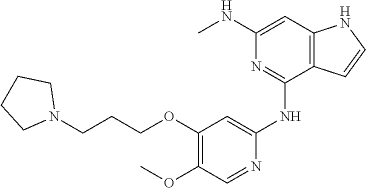 [SMILES: CNc1cc2[nH]ccc2c(Nc2cc(OCCCN3CCCC3)c(OC)cn2)n1] |
| 609 | 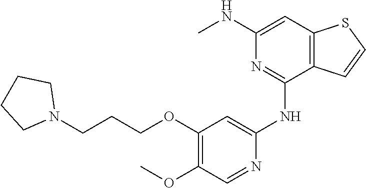 [SMILES: CNc1cc2sccc2c(Nc2cc(OCCCN3CCCC3)c(OC)cn2)n1] |
| 610 | 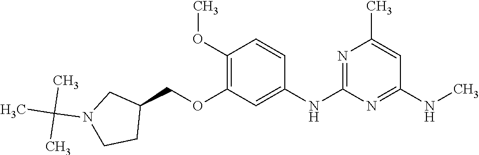 [SMILES: CNc1cc(C)nc(Nc2ccc(OC)c(OC[C@H]3CCN(C(C)(C)C)C3)c2)n1] |
| 611 | 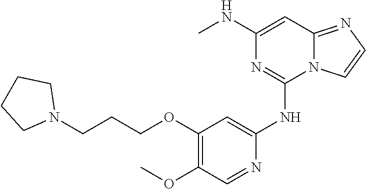 [SMILES: CNc1cc2nccn2c(Nc2cc(OCCCN3CCCC3)c(OC)cn2)n1] |
| 612 | 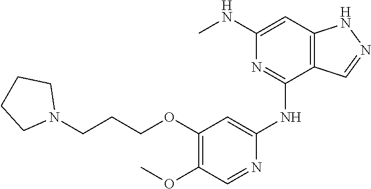 [SMILES: CNc1cc2[nH]ncc2c(Nc2cc(OCCCN3CCCC3)c(OC)cn2)n1] |
| 613 | 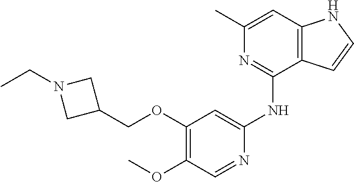 [SMILES: CCN1CC(COc2cc(Nc3nc(C)cc4[nH]ccc34)ncc2OC)C1] |
| 614 | 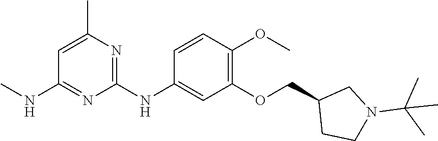 [SMILES: CNc1cc(C)nc(Nc2ccc(OC)c(OC[C@@H]3CCN(C(C)(C)C)C3)c2)n1] |
| 616 | 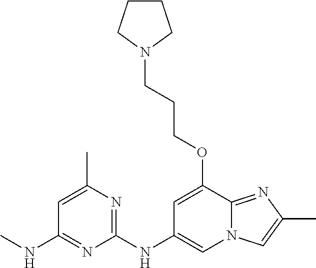 |
| 617 | 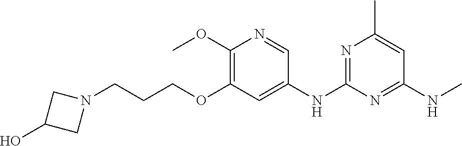 [SMILES: CNc1cc(C)nc(Nc2cnc(OC)c(OCCCN3CC(O)C3)c2)n1] |
| 618 | 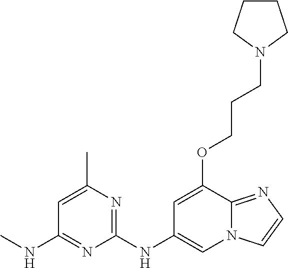 [SMILES: CNc1cc(C)nc(Nc2cc(OCCCN3CCCC3)c3nccn3c2)n1] |
| 619 | 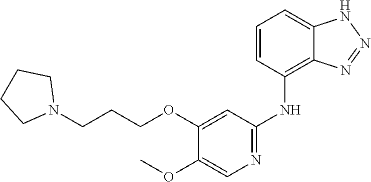 [SMILES: COc1cnc(Nc2cccc3[nH]nnc23)cc1OCCCN1CCCC1] |
| 620 | 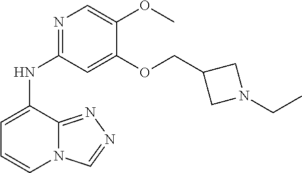 [SMILES: CCN1CC(COc2cc(Nc3cccn4cnnc34)ncc2OC)C1] |
| 621 | 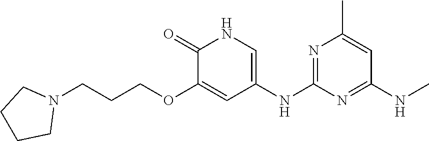 [SMILES: CNc1cc(C)nc(Nc2c[nH]c(=O)c(OCCCN3CCCC3)c2)n1] |
| 622 | 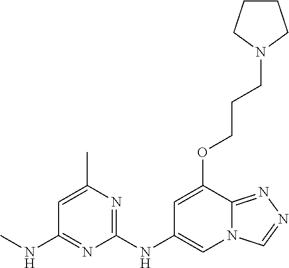 [SMILES: CNc1cc(C)nc(Nc2cc(OCCCN3CCCC3)c3nncn3c2)n1] |
| 623 | 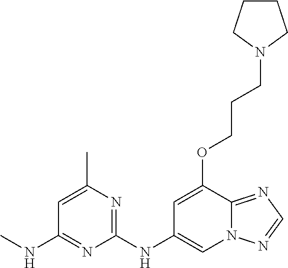 [SMILES: CNc1cc(C)nc(Nc2cc(OCCCN3CCCC3)c3ncnn3c2)n1] |
| 624 | 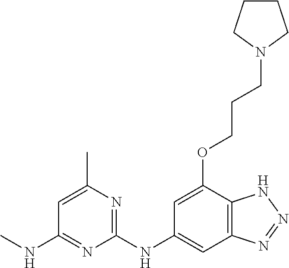 [SMILES: CNc1cc(C)nc(Nc2cc(OCCCN3CCCC3)c3[nH]nnc3c2)n1] |
| 625 | 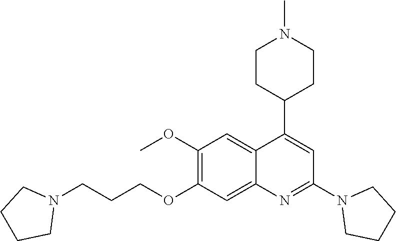 [SMILES: COc1cc2c(C3CCN(C)CC3)cc(N3CCCC3)nc2cc1OCCCN1CCCC1] |
| 626 | 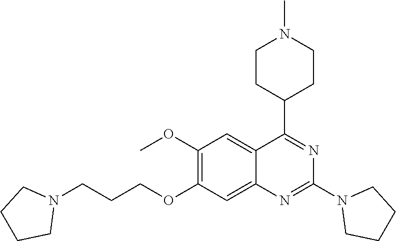 [SMILES: COc1cc2c(C3CCN(C)CC3)nc(N3CCCC3)nc2cc1OCCCN1CCCC1] |
| 627 | 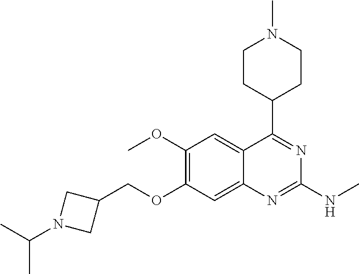 [SMILES: CNc1nc(C2CCN(C)CC2)c2cc(OC)c(OCC3CN(C(C)C)C3)cc2n1] |
| 628 | 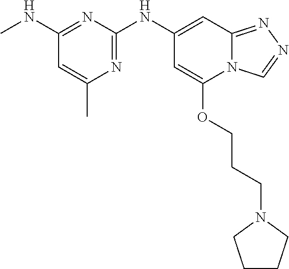 [SMILES: CNc1cc(C)nc(Nc2cc(OCCCN3CCCC3)n3cnnc3c2)n1] |
| 629 | 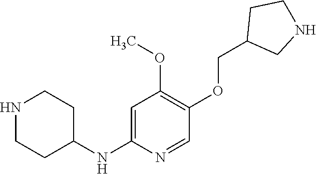 [SMILES: COc1cc(NC2CCNCC2)ncc1OCC1CCNC1] |
| 630 | 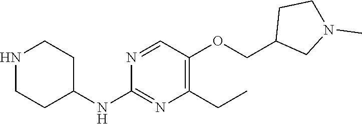 [SMILES: CCc1nc(NC2CCNCC2)ncc1OCC1CCN(C)C1] |
| 631 | 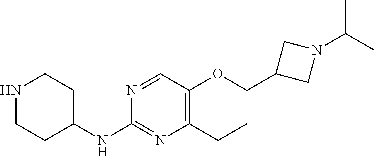 [SMILES: CCc1nc(NC2CCNCC2)ncc1OCC1CN(C(C)C)C1] |
| 632 | 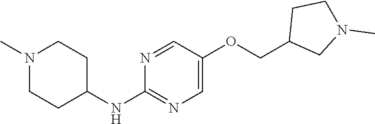 |
| 633 | 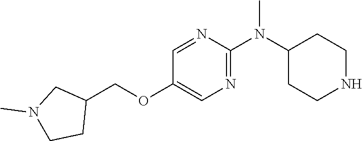 [SMILES: CN1CCC(COc2cnc(N(C)C3CCNCC3)nc2)C1] |
| 634 | 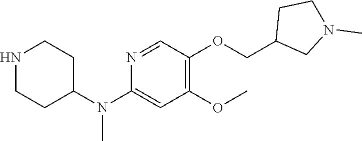 [SMILES: COc1cc(N(C)C2CCNCC2)ncc1OCC1CCN(C)C1] |
| 635 | 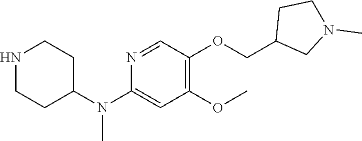 [SMILES: COc1cc(N(C)C2CCNCC2)ncc1OCC1CCN(C)C1] |
| 636 | 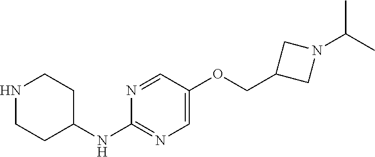 [SMILES: CC(C)N1CC(COc2cnc(NC3CCNCC3)nc2)C1] |
| 637 | 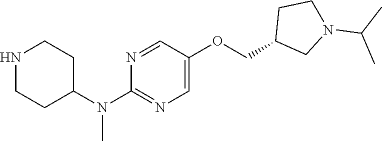 [SMILES: CC(C)N1CC[C@@H](COc2cnc(N(C)C3CCNCC3)nc2)C1] |
| 638 | 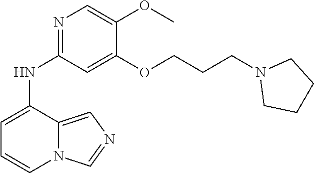 [SMILES: COc1cnc(Nc2cccn3cncc23)cc1OCCCN1CCCC1] |
| 639 | 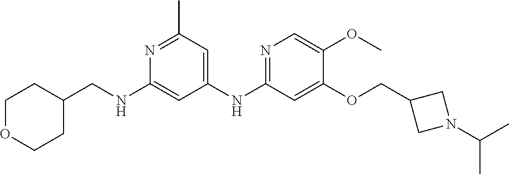 [SMILES: COc1cnc(Nc2cc(C)nc(NCC3CCOCC3)c2)cc1OCC1CN(C(C)C)C1] |
| 640 | 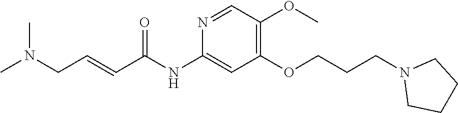 [SMILES: COc1cnc(NC(=O)/C=C/CN(C)C)cc1OCCCN1CCCC1] |
| 641 | 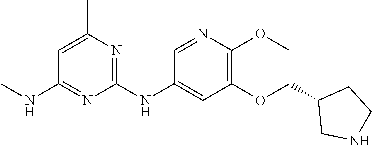 [SMILES: CNc1cc(C)nc(Nc2cnc(OC)c(OC[C@@H]3CCNC3)c2)n1] |
| 642 | 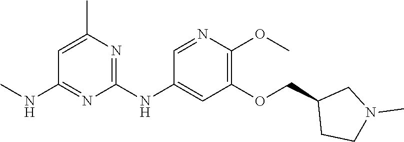 |
| 643 | 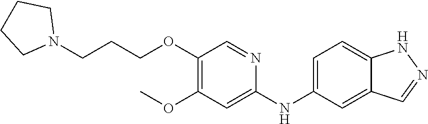 [SMILES: COc1cc(Nc2ccc3[nH]ncc3c2)ncc1OCCCN1CCCC1] |
| 644 | 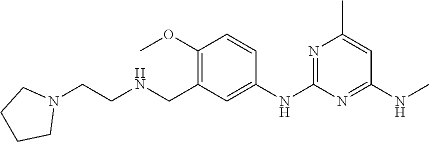 [SMILES: CNc1cc(C)nc(Nc2ccc(OC)c(CNCCN3CCCC3)c2)n1] |
| 645 | 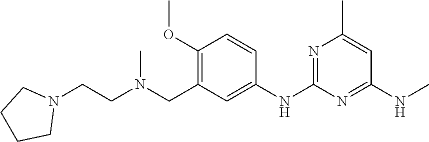 [SMILES: CNc1cc(C)nc(Nc2ccc(OC)c(CN(C)CCN3CCCC3)c2)n1] |
| 646 | 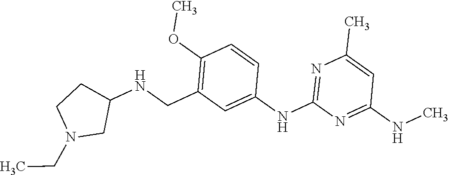 [SMILES: CCN1CCC(NCc2cc(Nc3nc(C)cc(NC)n3)ccc2OC)C1] |
| 647 | 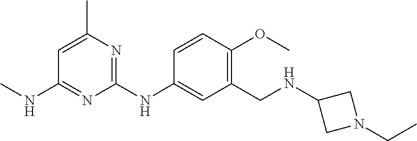 [SMILES: CCN1CC(NCc2cc(Nc3nc(C)cc(NC)n3)ccc2OC)C1] |
| 648 | 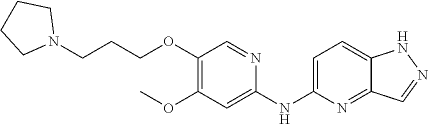 [SMILES: COc1cc(Nc2ccc3[nH]ncc3n2)ncc1OCCCN1CCCC1] |
| 649 | 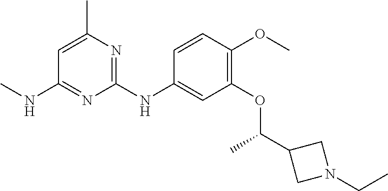 [SMILES: CCN1CC([C@H](C)Oc2cc(Nc3nc(C)cc(NC)n3)ccc2OC)C1] |
| 650 | 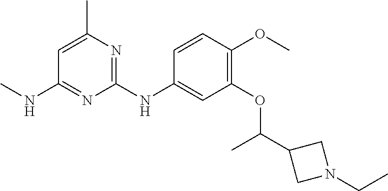 [SMILES: CCN1CC(C(C)Oc2cc(Nc3nc(C)cc(NC)n3)ccc2OC)C1] |
| 651 | 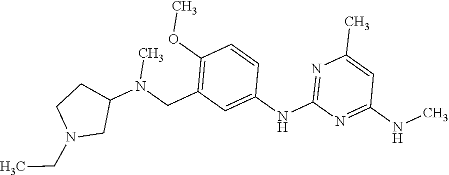 [SMILES: CCN1CCC(N(C)Cc2cc(Nc3nc(C)cc(NC)n3)ccc2OC)C1] |
| 652 | 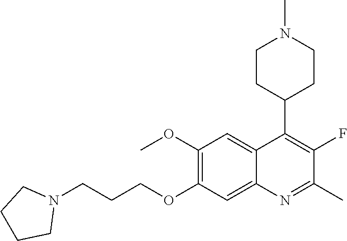 [SMILES: COc1cc2c(C3CCN(C)CC3)c(F)c(C)nc2cc1OCCCN1CCCC1] |
| 653 | 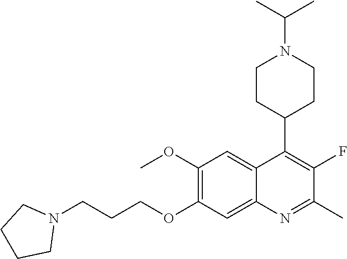 [SMILES: COc1cc2c(C3CCN(C(C)C)CC3)c(F)c(C)nc2cc1OCCCN1CCCC1] |
| 654 | 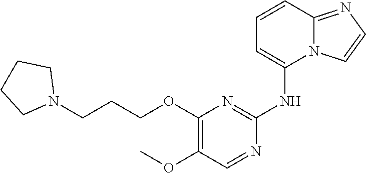 [SMILES: COc1cnc(Nc2cccc3nccn23)nc1OCCCN1CCCC1] |
| 655 | 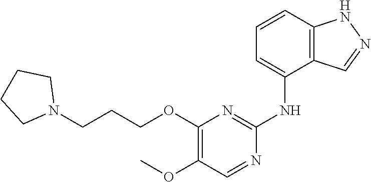 [SMILES: COc1cnc(Nc2cccc3[nH]ncc23)nc1OCCCN1CCCC1] |
| 656 | 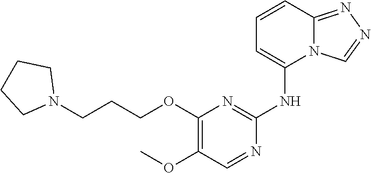 [SMILES: COc1cnc(Nc2cccc3nncn23)nc1OCCCN1CCCC1] |
| 657 | 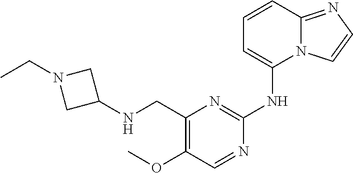 [SMILES: CCN1CC(NCc2nc(Nc3cccc4nccn34)ncc2OC)C1] |
| 658 | 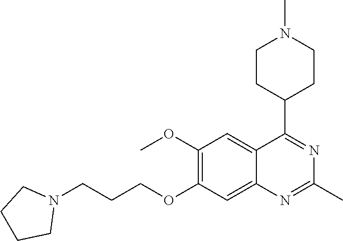 [SMILES: COc1cc2c(C3CCN(C)CC3)nc(C)nc2cc1OCCCN1CCCC1] |
| 659 | 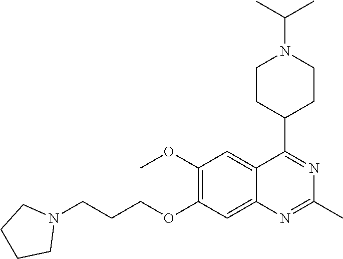 [SMILES: COc1cc2c(C3CCN(C(C)C)CC3)nc(C)nc2cc1OCCCN1CCCC1] |
| 660 | 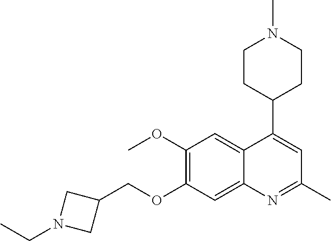 [SMILES: CCN1CC(COc2cc3nc(C)cc(C4CCN(C)CC4)c3cc2OC)C1] |
| 661 | 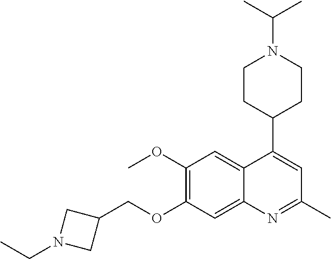 [SMILES: CCN1CC(COc2cc3nc(C)cc(C4CCN(C(C)C)CC4)c3cc2OC)C1] |
| 662 | 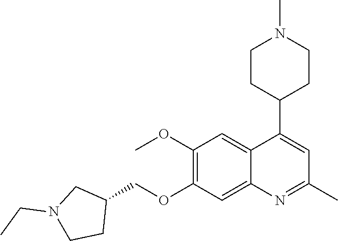 [SMILES: CCN1CC[C@@H](COc2cc3nc(C)cc(C4CCN(C)CC4)c3cc2OC)C1] |
| 663 | 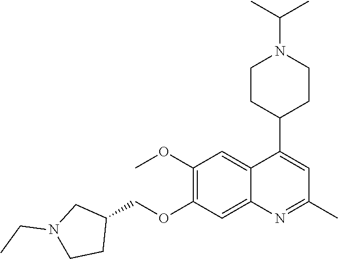 [SMILES: CCN1CC[C@@H](COc2cc3nc(C)cc(C4CCN(C(C)C)CC4)c3cc2OC)C1] |
| 664 | 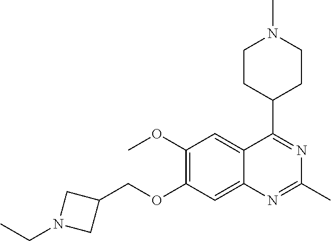 [SMILES: CCN1CC(COc2cc3nc(C)nc(C4CCN(C)CC4)c3cc2OC)C1] |
| 665 | 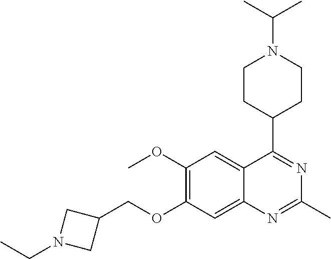 [SMILES: CCN1CC(COc2cc3nc(C)nc(C4CCN(C(C)C)CC4)c3cc2OC)C1] |
| 666 | 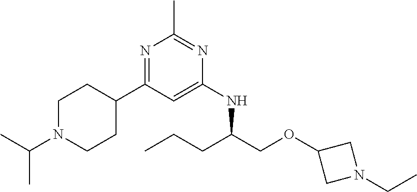 [SMILES: CCC[C@H](COC1CN(CC)C1)Nc1cc(C2CCN(C(C)C)CC2)nc(C)n1] |
| 667 | 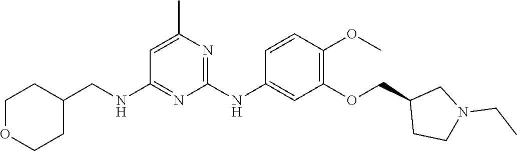 [SMILES: CCN1CC[C@@H](COc2cc(Nc3nc(C)cc(NCC4CCOCC4)n3)ccc2OC)C1] |
| 668 | 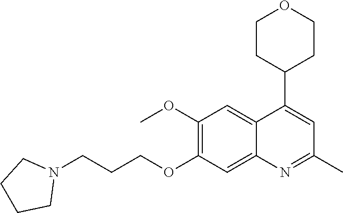 [SMILES: COc1cc2c(C3CCOCC3)cc(C)nc2cc1OCCCN1CCCC1] |
| 669 | 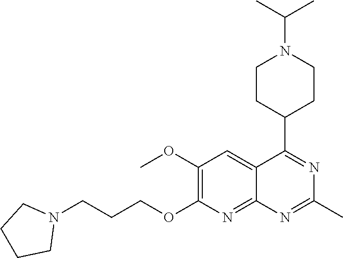 [SMILES: COc1cc2c(C3CCN(C(C)C)CC3)nc(C)nc2nc1OCCCN1CCCC1] |
| 670 | 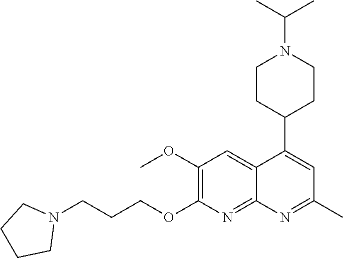 [SMILES: COc1cc2c(C3CCN(C(C)C)CC3)cc(C)nc2nc1OCCCN1CCCC1] |
| 671 | 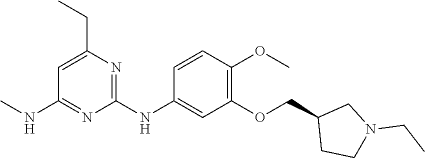 [SMILES: CCc1cc(NC)nc(Nc2ccc(OC)c(OC[C@@H]3CCN(CC)C3)c2)n1] |
| 672 | 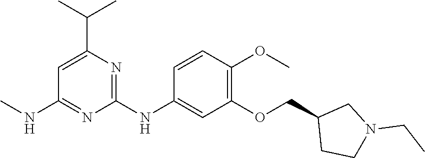 [SMILES: CCN1CC[C@@H](COc2cc(Nc3nc(NC)cc(C(C)C)n3)ccc2OC)C1] |
| 673 | 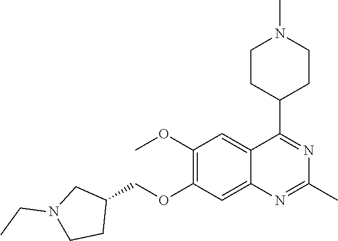 [SMILES: CCN1CC[C@@H](COc2cc3nc(C)nc(C4CCN(C)CC4)c3cc2OC)C1] |
| 674 | 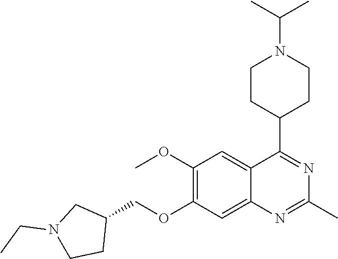 [SMILES: CCN1CC[C@@H](COc2cc3nc(C)nc(C4CCN(C(C)C)CC4)c3cc2OC)C1] |
| 675 | 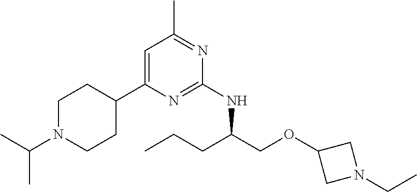 [SMILES: CCC[C@H](COC1CN(CC)C1)Nc1nc(C)cc(C2CCN(C(C)C)CC2)n1] |
| 676 | 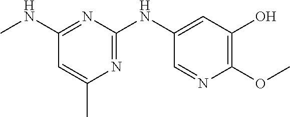 |
| 677 | 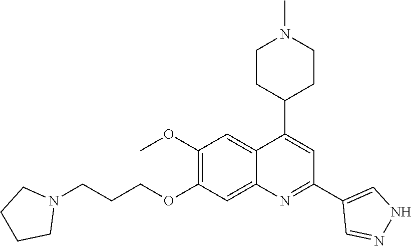 [SMILES: COc1cc2c(C3CCN(C)CC3)cc(-c3cn[nH]c3)nc2cc1OCCCN1CCCC1] |
| 678 | 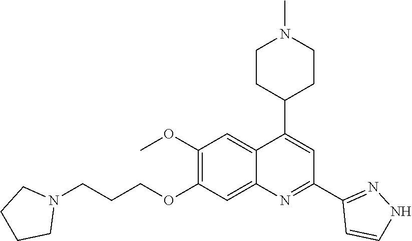 [SMILES: COc1cc2c(C3CCN(C)CC3)cc(-c3cc[nH]n3)nc2cc1OCCCN1CCCC1] |
| 679 | 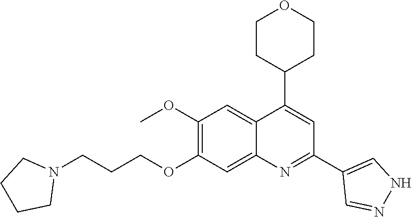 [SMILES: COc1cc2c(C3CCOCC3)cc(-c3cn[nH]c3)nc2cc1OCCCN1CCCC1] |
| 680 | 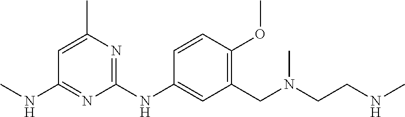 |
| 681 | 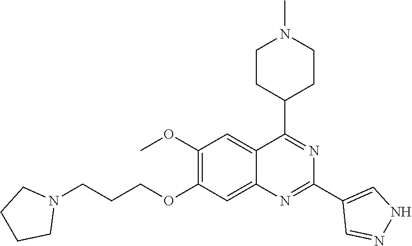 [SMILES: COc1cc2c(C3CCN(C)CC3)nc(-c3cn[nH]c3)nc2cc1OCCCN1CCCC1] |
| 682 | 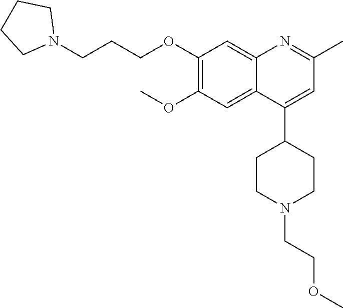 [SMILES: COCCN1CCC(c2cc(C)nc3cc(OCCCN4CCCC4)c(OC)cc23)CC1] |
| 683 | 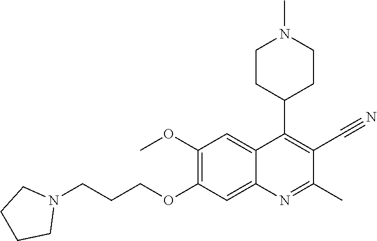 [SMILES: COc1cc2c(C3CCN(C)CC3)c(C#N)c(C)nc2cc1OCCCN1CCCC1] |
| 684 | 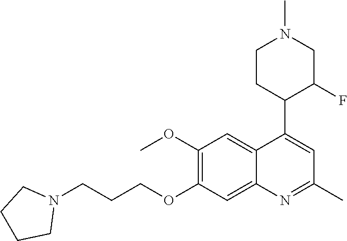 [SMILES: COc1cc2c(C3CCN(C)CC3F)cc(C)nc2cc1OCCCN1CCCC1] |
| 685 | 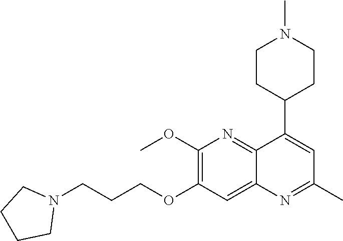 [SMILES: COc1nc2c(C3CCN(C)CC3)cc(C)nc2cc1OCCCN1CCCC1] |
| 686 | 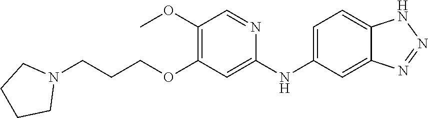 [SMILES: COc1cnc(Nc2ccc3[nH]nnc3c2)cc1OCCCN1CCCC1] |
| 687 | 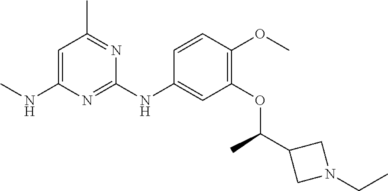 [SMILES: CCN1CC([C@@H](C)Oc2cc(Nc3nc(C)cc(NC)n3)ccc2OC)C1] |
| 688 | 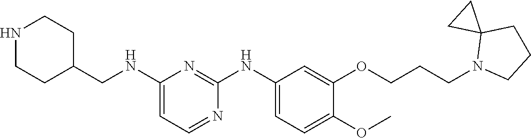 [SMILES: COc1ccc(Nc2nccc(NCC3CCNCC3)n2)cc1OCCCN1CCCC12CC2] |
| 689 | 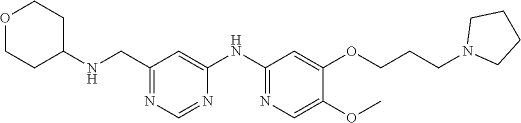 [SMILES: COc1cnc(Nc2cc(CNC3CCOCC3)ncn2)cc1OCCCN1CCCC1] |
| 690 | 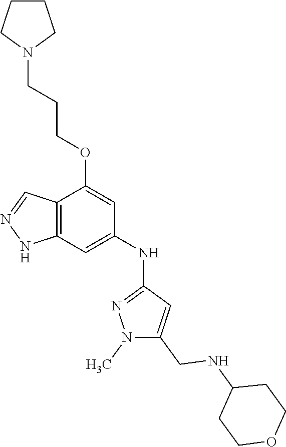 [SMILES: Cn1nc(Nc2cc(OCCCN3CCCC3)c3cn[nH]c3c2)cc1CNC1CCOCC1] |
| 691 | 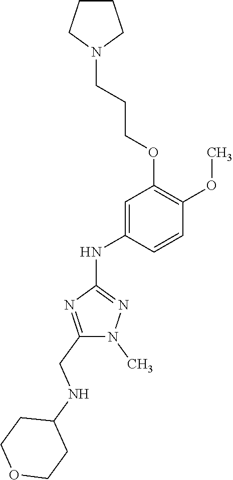 [SMILES: COc1ccc(Nc2nc(CNC3CCOCC3)n(C)n2)cc1OCCCN1CCCC1] |
| 692 | 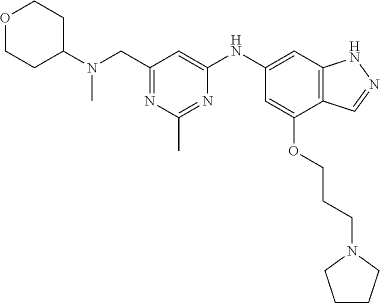 [SMILES: Cc1nc(CN(C)C2CCOCC2)cc(Nc2cc(OCCCN3CCCC3)c3cn[nH]c3c2)n1] |
| 693 | 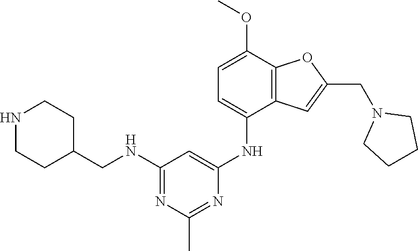 [SMILES: COc1ccc(Nc2cc(NCC3CCNCC3)nc(C)n2)c2cc(CN3CCCC3)oc12] |
| 694 | 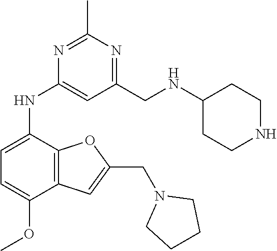 [SMILES: COc1ccc(Nc2cc(CNC3CCNCC3)nc(C)n2)c2oc(CN3CCCC3)cc12] |
| 695 | 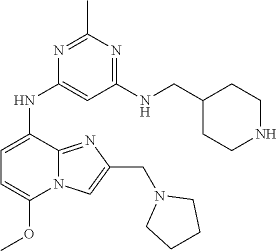 [SMILES: COc1ccc(Nc2cc(NCC3CCNCC3)nc(C)n2)c2nc(CN3CCCC3)cn12] |
| 696 | 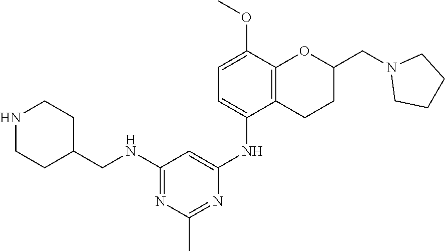 [SMILES: COc1ccc(Nc2cc(NCC3CCNCC3)nc(C)n2)c2c1OC(CN1CCCC1)CC2] |
| 697 | 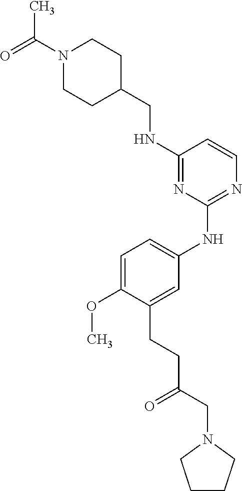 [SMILES: COc1ccc(Nc2nccc(NCC3CCN(C(C)=O)CC3)n2)cc1CCC(=O)CN1CCCC1] |
| 698 | 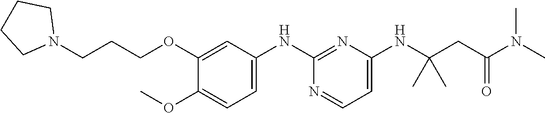 [SMILES: COc1ccc(Nc2nccc(NC(C)(C)CC(=O)N(C)C)n2)cc1OCCCN1CCCC1] |
| 699 | 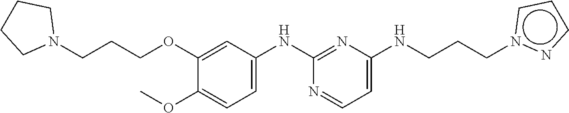 |
| 700 | 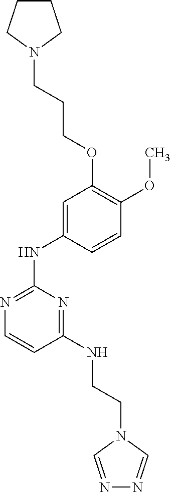 [SMILES: COc1ccc(Nc2nccc(NCCn3cnnc3)n2)cc1OCCCN1CCCC1] |
| 701 | 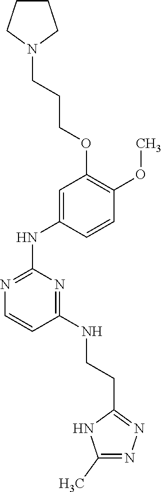 [SMILES: COc1ccc(Nc2nccc(NCCc3nnc(C)[nH]3)n2)cc1OCCCN1CCCC1] |
| 702 | 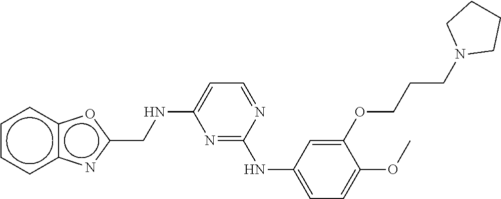 |
| 703 | 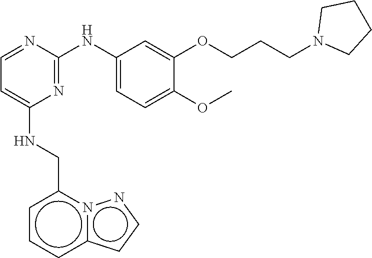 |
| 704 | 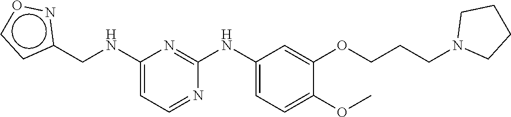 |
| 705 | 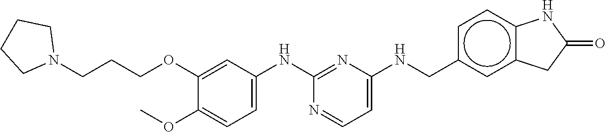 |
| 706 | 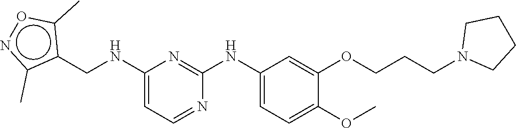 |
| 707 | 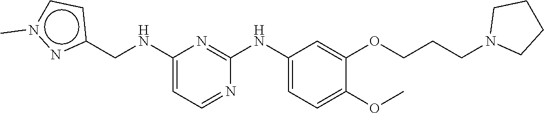 |
| 708 | 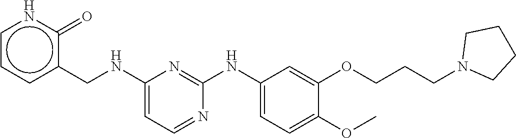 |
| 709 | 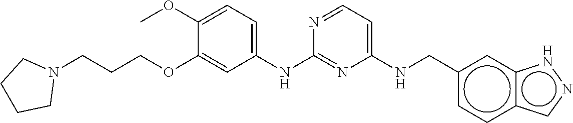 |
| 710 | 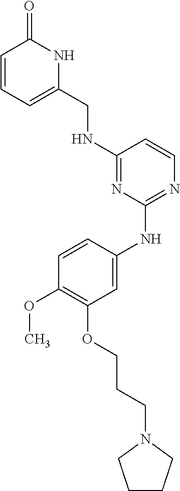 [SMILES: COc1ccc(Nc2nccc(NCc3cccc(=O)[nH]3)n2)cc1OCCCN1CCCC1] |
| 711 | 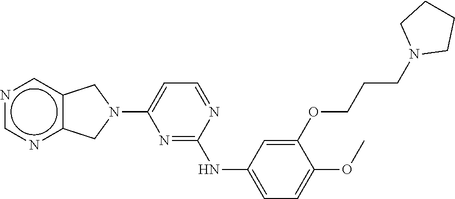 |
| 712 | 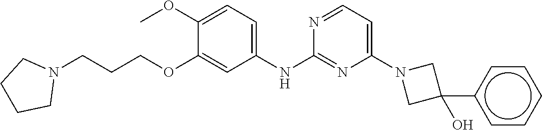 |
| 713 | 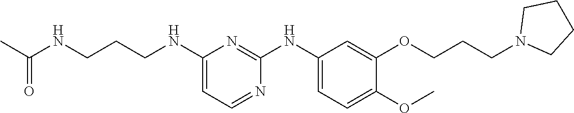 [SMILES: COc1ccc(Nc2nccc(NCCCNC(C)=O)n2)cc1OCCCN1CCCC1] |
| 714 | 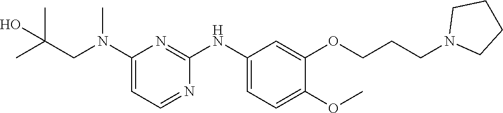 [SMILES: COc1ccc(Nc2nccc(N(C)CC(C)(C)O)n2)cc1OCCCN1CCCC1] |
| 715 | 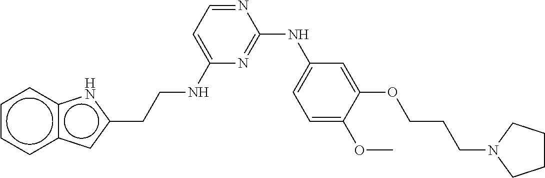 |
| 716 | 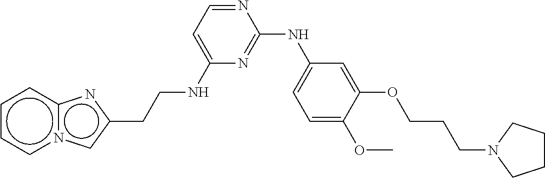 |
| 717 | 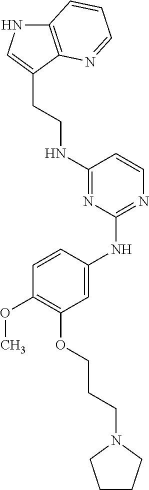 [SMILES: COc1ccc(Nc2nccc(NCCc3c[nH]c4cccnc34)n2)cc1OCCCN1CCCC1] |
| 718 | 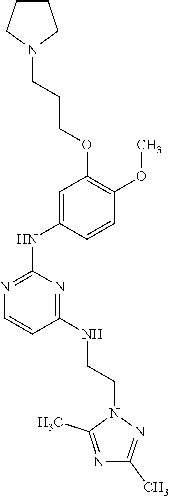 [SMILES: COc1ccc(Nc2nccc(NCCn3nc(C)nc3C)n2)cc1OCCCN1CCCC1] |
| 719 | 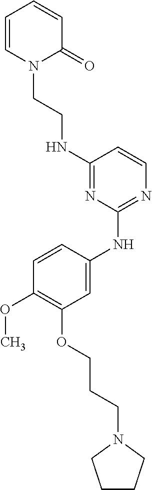 [SMILES: COc1ccc(Nc2nccc(NCCn3ccccc3=O)n2)cc1OCCCN1CCCC1] |
| 720 | 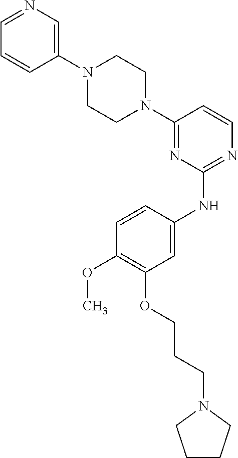 [SMILES: COc1ccc(Nc2nccc(N3CCN(c4cccnc4)CC3)n2)cc1OCCCN1CCCC1] |
| 721 | 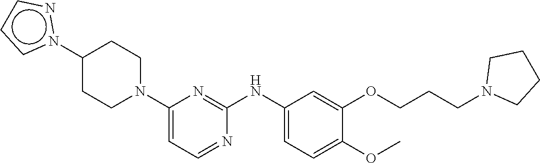 |
| 722 | 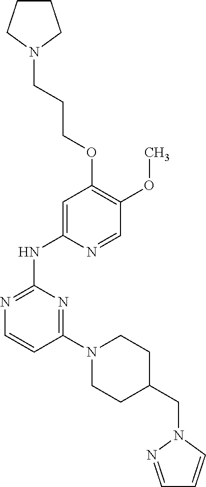 [SMILES: COc1cnc(Nc2nccc(N3CCC(Cn4cccn4)CC3)n2)cc1OCCCN1CCCC1] |
| 723 | 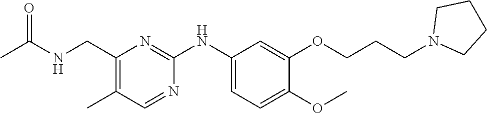 [SMILES: COc1ccc(Nc2ncc(C)c(CNC(C)=O)n2)cc1OCCCN1CCCC1] |
| 724 | 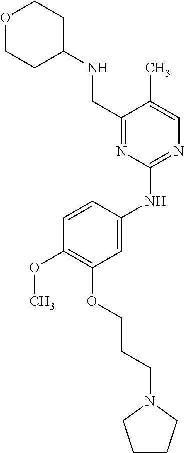 [SMILES: COc1ccc(Nc2ncc(C)c(CNC3CCOCC3)n2)cc1OCCCN1CCCC1] |
| 725 | 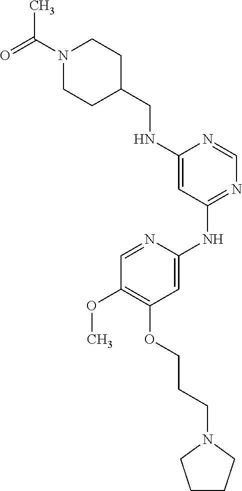 [SMILES: COc1cnc(Nc2cc(NCC3CCN(C(C)=O)CC3)ncn2)cc1OCCCN1CCCC1] |
| 726 | 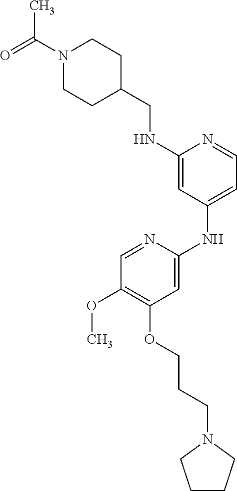 [SMILES: COc1cnc(Nc2ccnc(NCC3CCN(C(C)=O)CC3)c2)cc1OCCCN1CCCC1] |
| 727 | 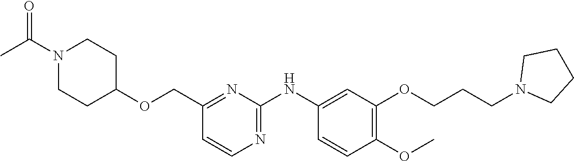 [SMILES: COc1ccc(Nc2nccc(COC3CCN(C(C)=O)CC3)n2)cc1OCCCN1CCCC1] |
| 728 | 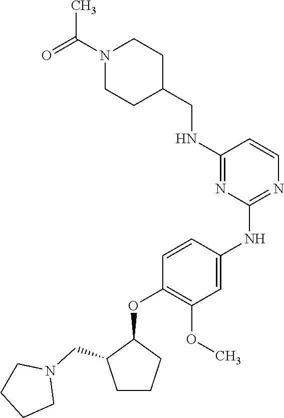 [SMILES: COc1cc(Nc2nccc(NCC3CCN(C(C)=O)CC3)n2)ccc1O[C@H]1CCC[C@@H]1CN1CCCC1] |
| 729 | 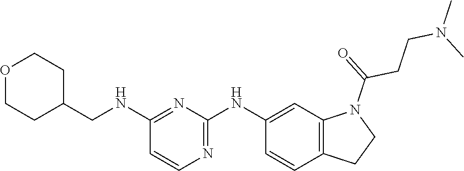 [SMILES: CN(C)CCC(=O)N1CCc2ccc(Nc3nccc(NCC4CCOCC4)n3)cc21] |
| 730 | 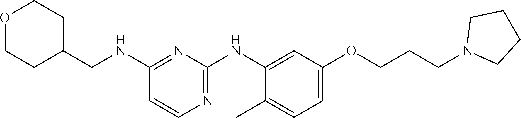 [SMILES: Cc1ccc(OCCCN2CCCC2)cc1Nc1nccc(NCC2CCOCC2)n1] |
| 731 | 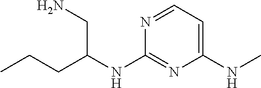 |
| 732 | 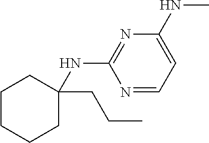 [SMILES: CCCC1(Nc2nccc(NC)n2)CCCCC1] |
| 733 | 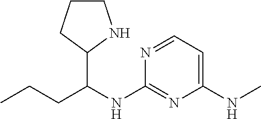 [SMILES: CCCC(Nc1nccc(NC)n1)C1CCCN1] |
| 734 | 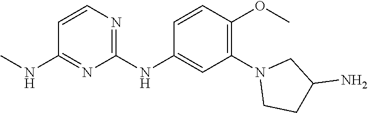 [SMILES: CNc1ccnc(Nc2ccc(OC)c(N3CCC(N)C3)c2)n1] |
| 735 | 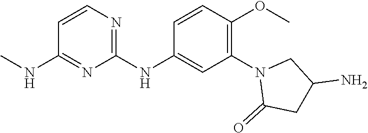 [SMILES: CNc1ccnc(Nc2ccc(OC)c(N3CC(N)CC3=O)c2)n1] |
| 736 | 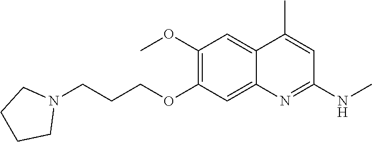 [SMILES: CNc1cc(C)c2cc(OC)c(OCCCN3CCCC3)cc2n1] |
| 737 | 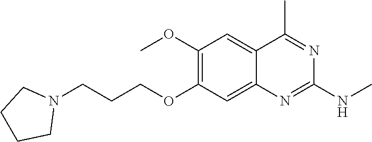 [SMILES: CNc1nc(C)c2cc(OC)c(OCCCN3CCCC3)cc2n1] |
| 738 | 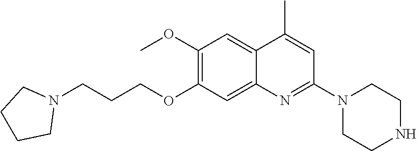 [SMILES: COc1cc2c(C)cc(N3CCNCC3)nc2cc1OCCCN1CCCC1] |
| 739 | 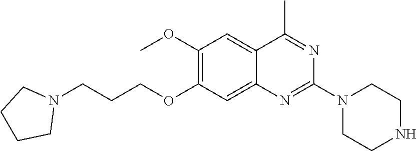 [SMILES: COc1cc2c(C)nc(N3CCNCC3)nc2cc1OCCCN1CCCC1] |
| 740 | 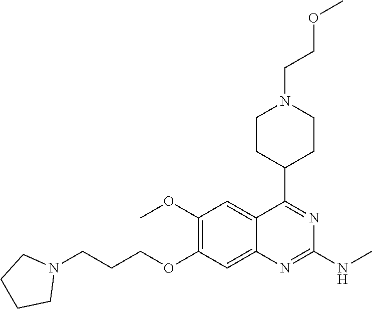 [SMILES: CNc1nc(C2CCN(CCOC)CC2)c2cc(OC)c(OCCCN3CCCC3)cc2n1] |
| 741 | 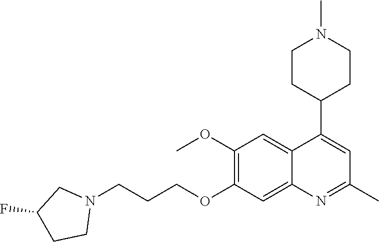 [SMILES: COc1cc2c(C3CCN(C)CC3)cc(C)nc2cc1OCCCN1CC[C@H](F)C1] |
| 742 | 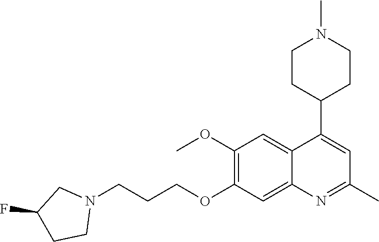 [SMILES: COc1cc2c(C3CCN(C)CC3)cc(C)nc2cc1OCCCN1CC[C@@H](F)C1] |
| 743 | 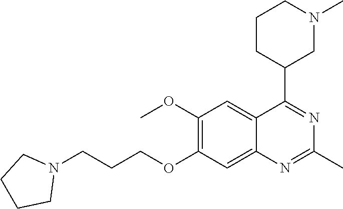 [SMILES: COc1cc2c(C3CCCN(C)C3)nc(C)nc2cc1OCCCN1CCCC1] |
| 744 | 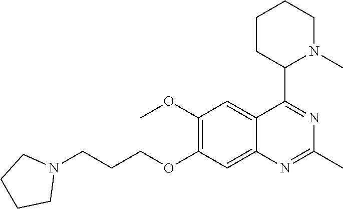 [SMILES: COc1cc2c(C3CCCCN3C)nc(C)nc2cc1OCCCN1CCCC1] |
| 745 | 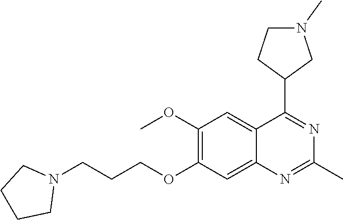 [SMILES: COc1cc2c(C3CCN(C)C3)nc(C)nc2cc1OCCCN1CCCC1] |
| 746 | 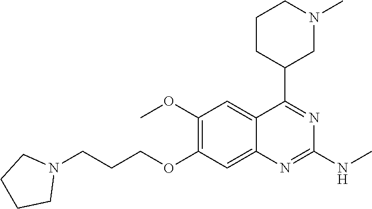 [SMILES: CNc1nc(C2CCCN(C)C2)c2cc(OC)c(OCCCN3CCCC3)cc2n1] |
| 747 | 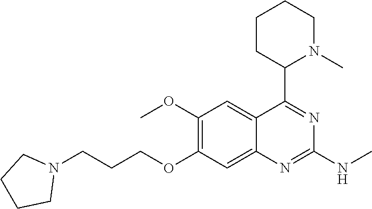 [SMILES: CNc1nc(C2CCCCN2C)c2cc(OC)c(OCCCN3CCCC3)cc2n1] |
| 748 | 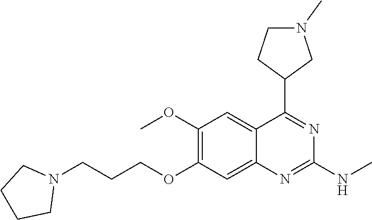 [SMILES: CNc1nc(C2CCN(C)C2)c2cc(OC)c(OCCCN3CCCC3)cc2n1] |
| 749 | 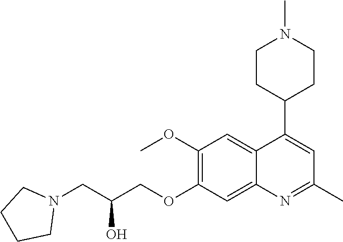 [SMILES: COc1cc2c(C3CCN(C)CC3)cc(C)nc2cc1OC[C@@H](O)CN1CCCC1] |
| 750 | 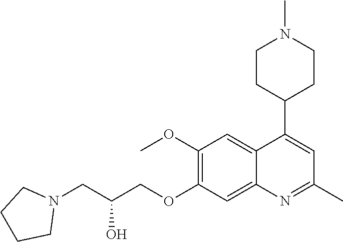 [SMILES: COc1cc2c(C3CCN(C)CC3)cc(C)nc2cc1OC[C@H](O)CN1CCCC1] |
| 751 | 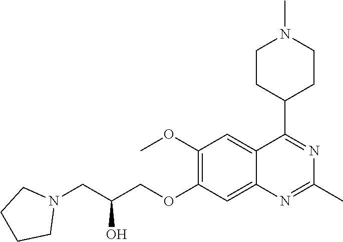 [SMILES: COc1cc2c(C3CCN(C)CC3)nc(C)nc2cc1OC[C@@H](O)CN1CCCC1] |
| 752 | 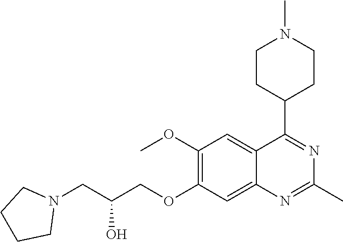 [SMILES: COc1cc2c(C3CCN(C)CC3)nc(C)nc2cc1OC[C@H](O)CN1CCCC1] |
| 753 | 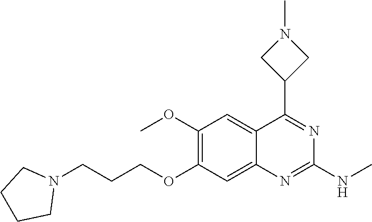 [SMILES: CNc1nc(C2CN(C)C2)c2cc(OC)c(OCCCN3CCCC3)cc2n1] |
| 754 | 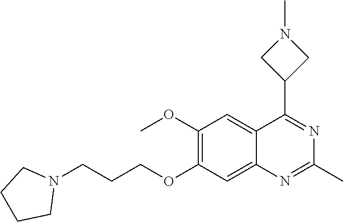 [SMILES: COc1cc2c(C3CN(C)C3)nc(C)nc2cc1OCCCN1CCCC1] |
| 755 | 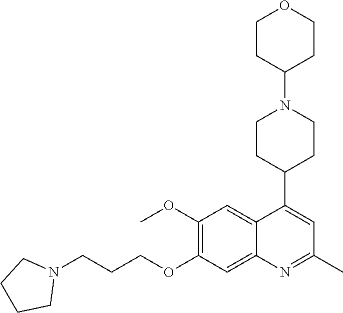 [SMILES: COc1cc2c(C3CCN(C4CCOCC4)CC3)cc(C)nc2cc1OCCCN1CCCC1] |
| 756 | 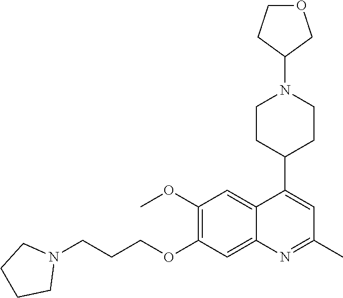 [SMILES: COc1cc2c(C3CCN(C4CCOC4)CC3)cc(C)nc2cc1OCCCN1CCCC1] |
| 757 | 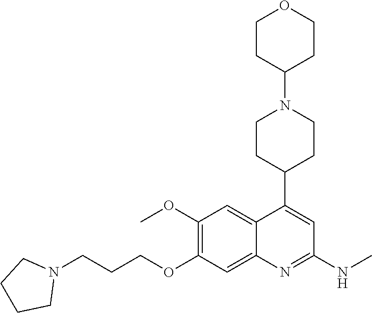 [SMILES: CNc1cc(C2CCN(C3CCOCC3)CC2)c2cc(OC)c(OCCCN3CCCC3)cc2n1] |
| 758 | 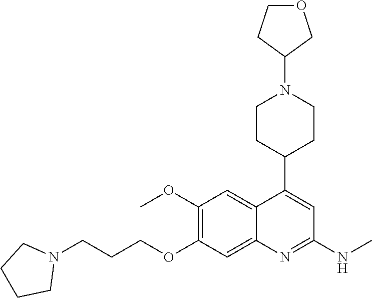 [SMILES: CNc1cc(C2CCN(C3CCOC3)CC2)c2cc(OC)c(OCCCN3CCCC3)cc2n1] |
| 759 | 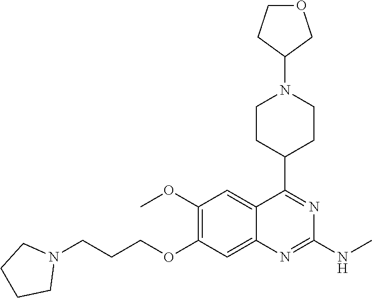 [SMILES: CNc1nc(C2CCN(C3CCOC3)CC2)c2cc(OC)c(OCCCN3CCCC3)cc2n1] |
| 760 | 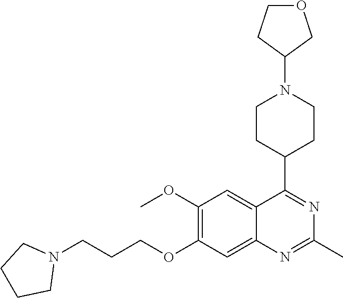 [SMILES: COc1cc2c(C3CCN(C4CCOC4)CC3)nc(C)nc2cc1OCCCN1CCCC1] |
| 761 | 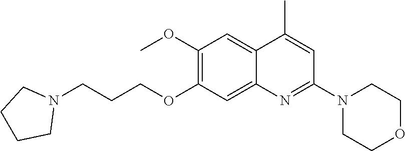 [SMILES: COc1cc2c(C)cc(N3CCOCC3)nc2cc1OCCCN1CCCC1] |
| 762 | 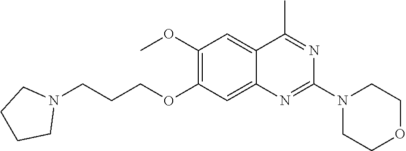 [SMILES: COc1cc2c(C)nc(N3CCOCC3)nc2cc1OCCCN1CCCC1] |
| 763 | 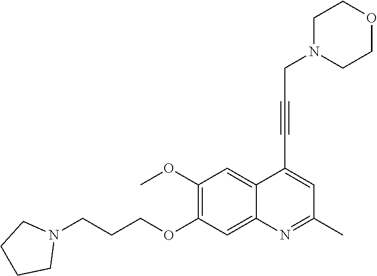 [SMILES: COc1cc2c(C#CCN3CCOCC3)cc(C)nc2cc1OCCCN1CCCC1] |
| 764 | 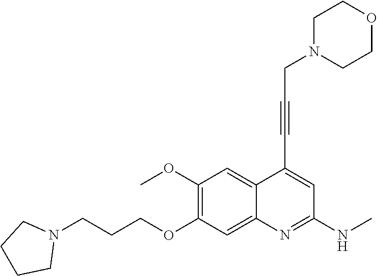 [SMILES: CNc1cc(C#CCN2CCOCC2)c2cc(OC)c(OCCCN3CCCC3)cc2n1] |
| 765 | 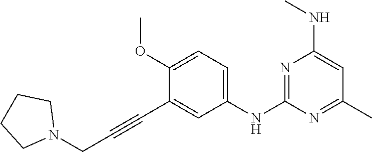 [SMILES: CNc1cc(C)nc(Nc2ccc(OC)c(C#CCN3CCCC3)c2)n1] |

TABLE 1D

| Cmpd. | |
|---|---|
| No. | Structure |
| 784 | 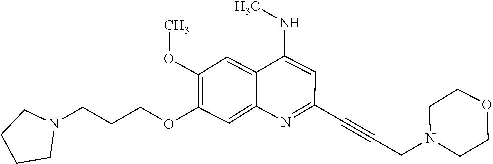 [SMILES: CNc1cc(C#CCN2CCOCC2)nc2cc(OCCCN3CCCC3)c(OC)cc12] |
| 786 | 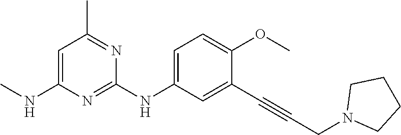 [SMILES: CNc1cc(C)nc(Nc2ccc(OC)c(C#CCN3CCCC3)c2)n1] |
| 787 | 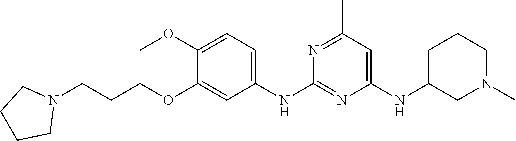 [SMILES: COc1ccc(Nc2nc(C)cc(NC3CCCN(C)C3)n2)cc1OCCCN1CCCC1] |
| 788 | 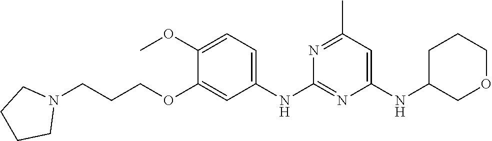 [SMILES: COc1ccc(Nc2nc(C)cc(NC3CCCOC3)n2)cc1OCCCN1CCCC1] |
| 789 | 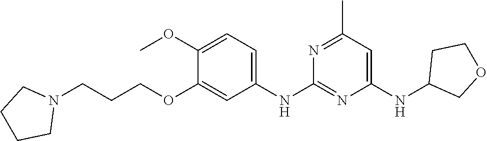 [SMILES: COc1ccc(Nc2nc(C)cc(NC3CCOC3)n2)cc1OCCCN1CCCC1] |
| 790 | 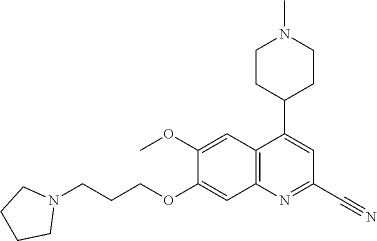 [SMILES: COc1cc2c(C3CCN(C)CC3)cc(C#N)nc2cc1OCCCN1CCCC1] |
| 791 | 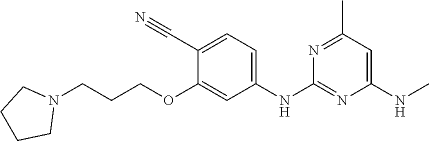 [SMILES: CNc1cc(C)nc(Nc2ccc(C#N)c(OCCCN3CCCC3)c2)n1] |
| 792 | 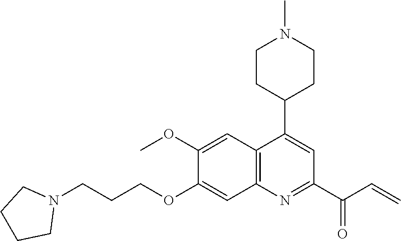 [SMILES: C=CC(=O)c1cc(C2CCN(C)CC2)c2cc(OC)c(OCCCN3CCCC3)cc2n1] |
| 793 | 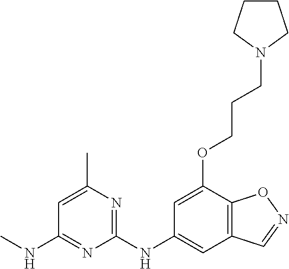 [SMILES: CNc1cc(C)nc(Nc2cc(OCCCN3CCCC3)c3oncc3c2)n1] |
| 794 | 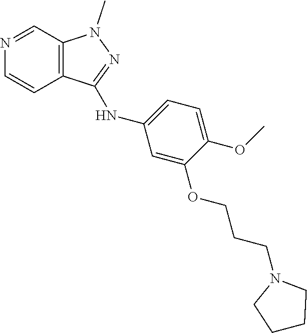 [SMILES: COc1ccc(Nc2nn(C)c3cnccc23)cc1OCCCN1CCCC1] |
| 795 | 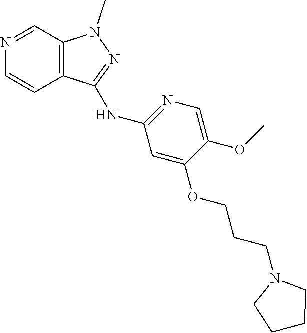 [SMILES: COc1cnc(Nc2nn(C)c3cnccc23)cc1OCCCN1CCCC1] |
| 796 | 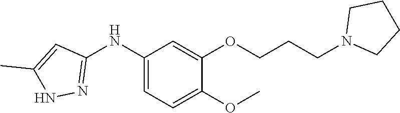 [SMILES: COc1ccc(Nc2cc(C)[nH]n2)cc1OCCCN1CCCC1] |
| 797 | 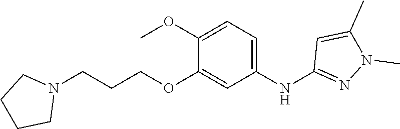 [SMILES: COc1ccc(Nc2cc(C)n(C)n2)cc1OCCCN1CCCC1] |
| 798 | 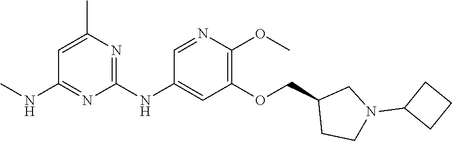 [SMILES: CNc1cc(C)nc(Nc2cnc(OC)c(OC[C@@H]3CCN(C4CCC4)C3)c2)n1] |
| 799 | 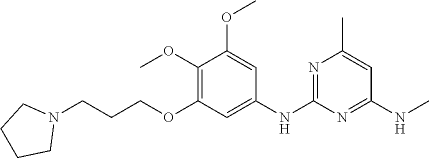 [SMILES: CNc1cc(C)nc(Nc2cc(OC)c(OC)c(OCCCN3CCCC3)c2)n1] |
| 800 | 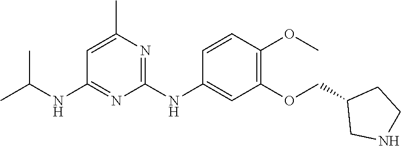 [SMILES: COc1ccc(Nc2nc(C)cc(NC(C)C)n2)cc1OC[C@@H]1CCNC1] |
| 801 | 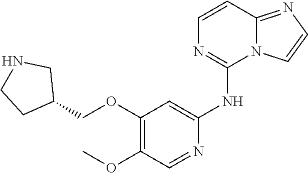 [SMILES: COc1cnc(Nc2nccc3nccn23)cc1OC[C@@H]1CCNC1] |
| 802 | 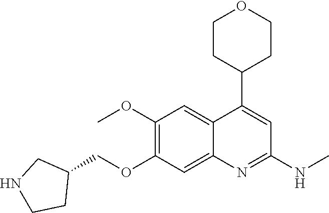 [SMILES: CNc1cc(C2CCOCC2)c2cc(OC)c(OC[C@@H]3CCNC3)cc2n1] |
| 803 | 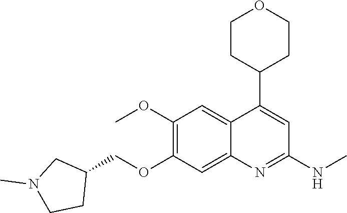 |
| 804 | 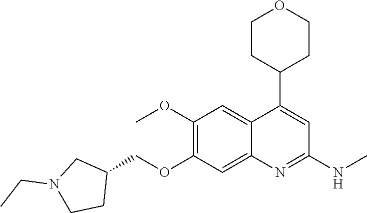 [SMILES: CCN1CC[C@@H](COc2cc3nc(NC)cc(C4CCOCC4)c3cc2OC)C1] |
| 805 | 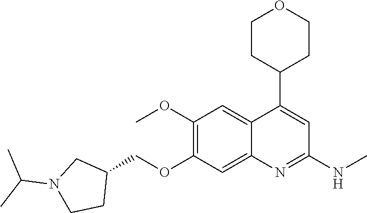 [SMILES: CNc1cc(C2CCOCC2)c2cc(OC)c(OC[C@@H]3CCN(C(C)C)C3)cc2n1] |
| 806 | 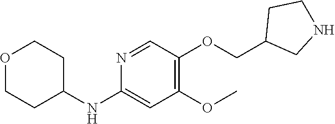 [SMILES: COc1cc(NC2CCOCC2)ncc1OCC1CCNC1] |
| 807 | 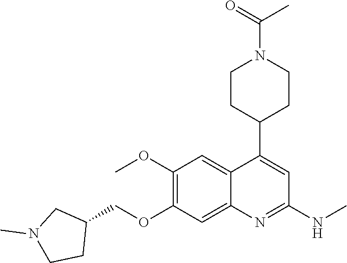 |
| 808 | 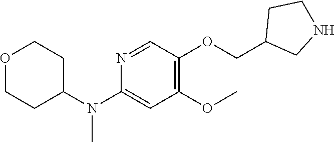 [SMILES: COc1cc(N(C)C2CCOCC2)ncc1OCC1CCNC1] |
| 809 | 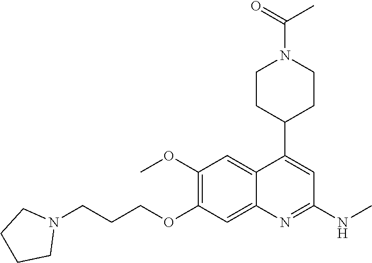 [SMILES: CNc1cc(C2CCN(C(C)=O)CC2)c2cc(OC)c(OCCCN3CCCC3)cc2n1] |
| 810 | 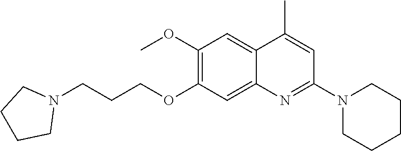 [SMILES: COc1cc2c(C)cc(N3CCCCC3)nc2cc1OCCCN1CCCC1] |
| 811 | 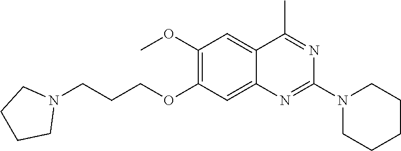 [SMILES: COc1cc2c(C)nc(N3CCCCC3)nc2cc1OCCCN1CCCC1] |
| 812 | 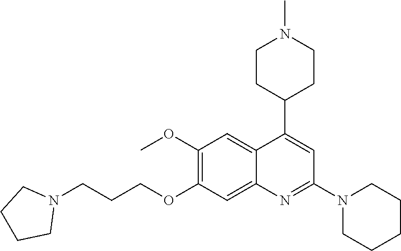 [SMILES: COc1cc2c(C3CCN(C)CC3)cc(N3CCCCC3)nc2cc1OCCCN1CCCC1] |
| 813 | 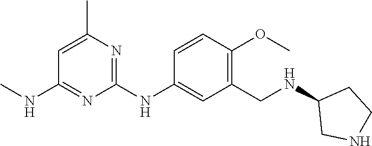 [SMILES: CNc1cc(C)nc(Nc2ccc(OC)c(CN[C@H]3CCNC3)c2)n1] |
| 814 | 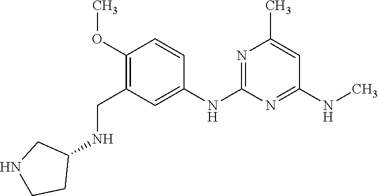 [SMILES: CNc1cc(C)nc(Nc2ccc(OC)c(CN[C@@H]3CCNC3)c2)n1] |
| 815 | 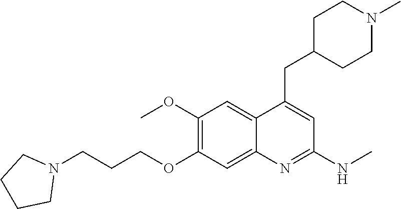 [SMILES: CNc1cc(CC2CCN(C)CC2)c2cc(OC)c(OCCCN3CCCC3)cc2n1] |
| 816 | 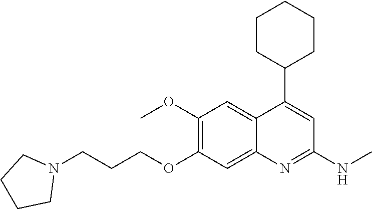 [SMILES: CNc1cc(C2CCCCC2)c2cc(OC)c(OCCCN3CCCC3)cc2n1] |
| 817 | 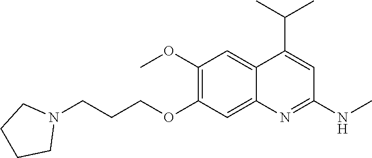 [SMILES: CNc1cc(C(C)C)c2cc(OC)c(OCCCN3CCCC3)cc2n1] |
| 820 | 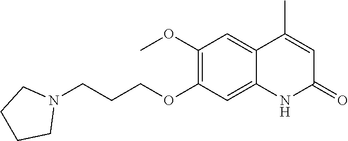 [SMILES: COc1cc2c(C)cc(=O)[nH]c2cc1OCCCN1CCCC1] |
| 821 | 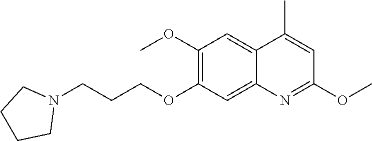 [SMILES: COc1cc(C)c2cc(OC)c(OCCCN3CCCC3)cc2n1] |
| 822 | 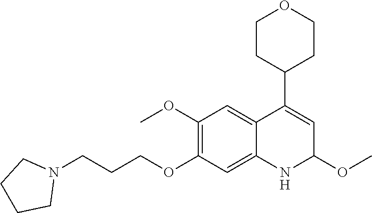 [SMILES: COc1cc2c(cc1OCCCN1CCCC1)NC(OC)C=C2C1CCOCC1] |
| 823 | 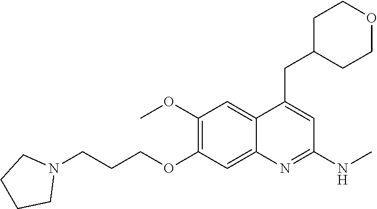 [SMILES: CNc1cc(CC2CCOCC2)c2cc(OC)c(OCCCN3CCCC3)cc2n1] |
| 824 | 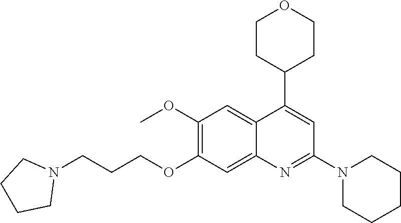 [SMILES: COc1cc2c(C3CCOCC3)cc(N3CCCCC3)nc2cc1OCCCN1CCCC1] |
| 825 | 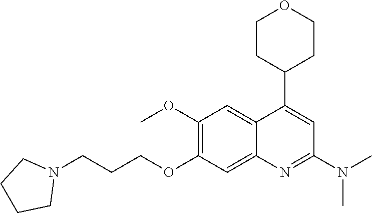 [SMILES: COc1cc2c(C3CCOCC3)cc(N(C)C)nc2cc1OCCCN1CCCC1] |
| 826 | 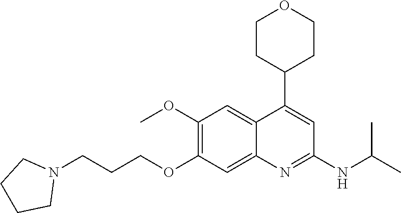 [SMILES: COc1cc2c(C3CCOCC3)cc(NC(C)C)nc2cc1OCCCN1CCCC1] |
| 827 | 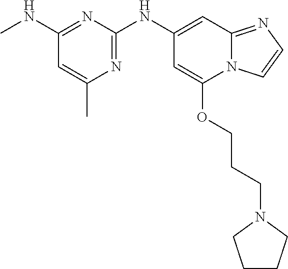 [SMILES: CNc1cc(C)nc(Nc2cc(OCCCN3CCCC3)n3ccnc3c2)n1] |
| 828 | 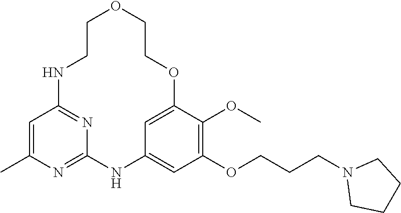 [SMILES: COc1c(OCCCN2CCCC2)cc2cc1OCCOCCNc1cc(C)nc(n1)N2] |
| 832 | 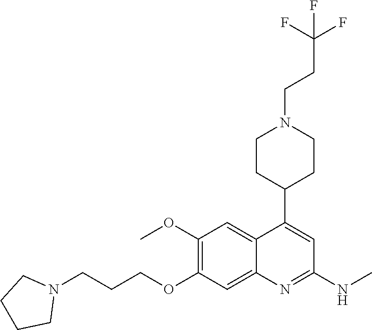 [SMILES: CNc1cc(C2CCN(CCC(F)(F)F)CC2)c2cc(OC)c(OCCCN3CCCC3)cc2n1] |
| 833 | 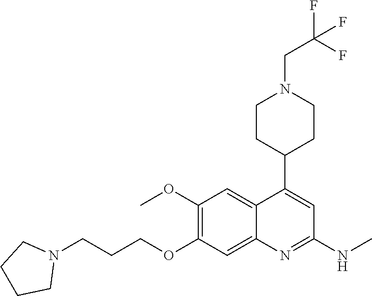 [SMILES: CNc1cc(C2CCN(CC(F)(F)F)CC2)c2cc(OC)c(OCCCN3CCCC3)cc2n1] |
| 834 | 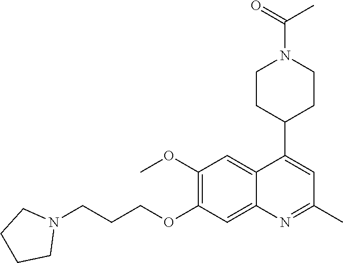 [SMILES: COc1cc2c(C3CCN(C(C)=O)CC3)cc(C)nc2cc1OCCCN1CCCC1] |
| 836 | 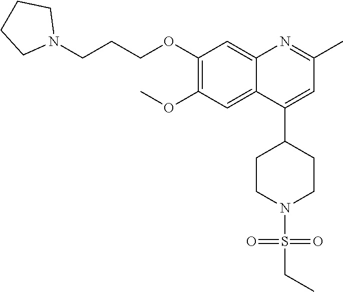 [SMILES: CCS(=O)(=O)N1CCC(c2cc(C)nc3cc(OCCCN4CCCC4)c(OC)cc23)CC1] |
| 837 | 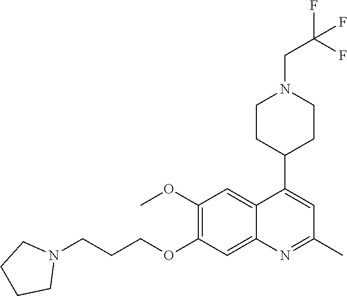 [SMILES: COc1cc2c(C3CCN(CC(F)(F)F)CC3)cc(C)nc2cc1OCCCN1CCCC1] |
| 838 | 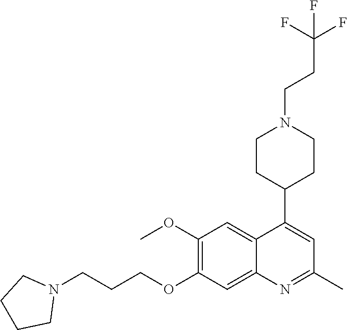 [SMILES: COc1cc2c(C3CCN(CCC(F)(F)F)CC3)cc(C)nc2cc1OCCCN1CCCC1] |
| 839 | 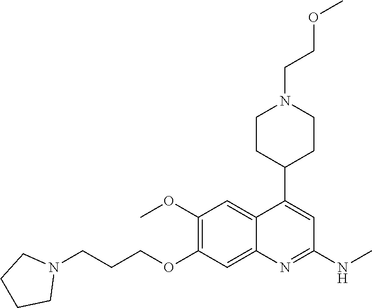 [SMILES: CNc1cc(C2CCN(CCOC)CC2)c2cc(OC)c(OCCCN3CCCC3)cc2n1] |
| 840 | 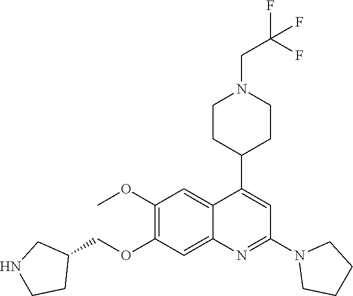 [SMILES: COc1cc2c(C3CCN(CC(F)(F)F)CC3)cc(N3CCCC3)nc2cc1OC[C@@H]1CCNC1] |
| 841 | 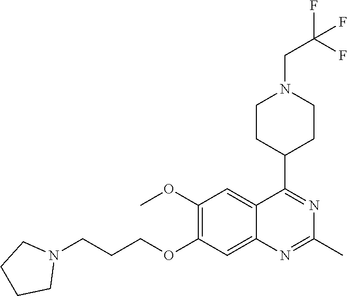 [SMILES: COc1cc2c(C3CCN(CC(F)(F)F)CC3)nc(C)nc2cc1OCCCN1CCCC1] |
| 842 | 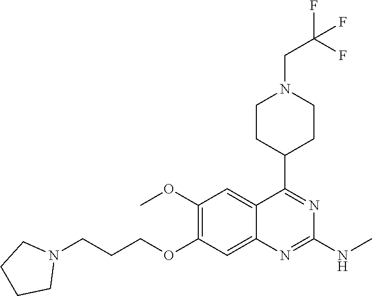 [SMILES: CNc1nc(C2CCN(CC(F)(F)F)CC2)c2cc(OC)c(OCCCN3CCCC3)cc2n1] |
| 844 | 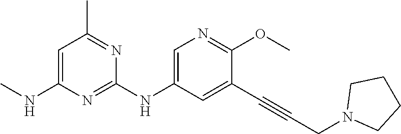 [SMILES: CNc1cc(C)nc(Nc2cnc(OC)c(C#CCN3CCCC3)c2)n1] |
| 845 | 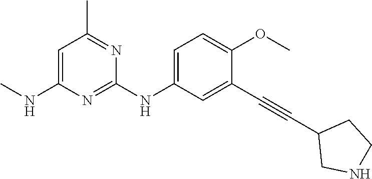 [SMILES: CNc1cc(C)nc(Nc2ccc(OC)c(C#CC3CCNC3)c2)n1] |
| 846 | 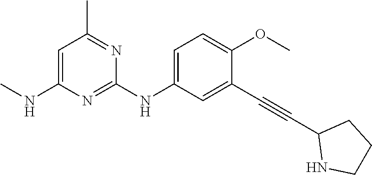 [SMILES: CNc1cc(C)nc(Nc2ccc(OC)c(C#CC3CCCN3)c2)n1] |
| 847 | 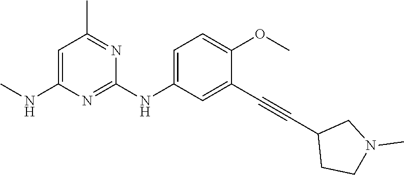 |
| 848 | 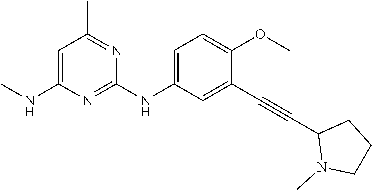 [SMILES: CNc1cc(C)nc(Nc2ccc(OC)c(C#CC3CCCN3C)c2)n1] |
| 849 | 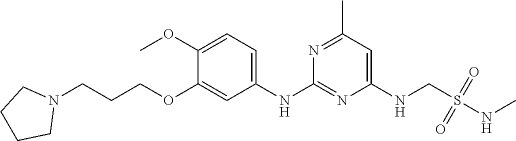 [SMILES: CNS(=O)(=O)CNc1cc(C)nc(Nc2ccc(OC)c(OCCCN3CCCC3)c2)n1] |
| 850 | 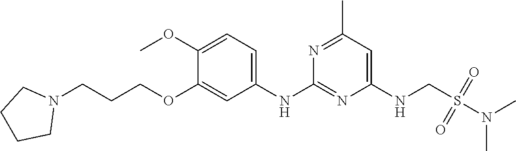 [SMILES: COc1ccc(Nc2nc(C)cc(NCS(=O)(=O)N(C)C)n2)cc1OCCCN1CCCC1] |
| 851 | 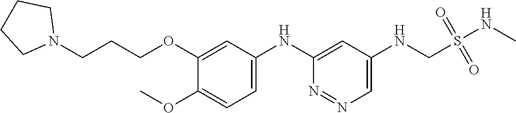 [SMILES: CNS(=O)(=O)CNc1cnnc(Nc2ccc(OC)c(OCCCN3CCCC3)c2)c1] |
| 852 | 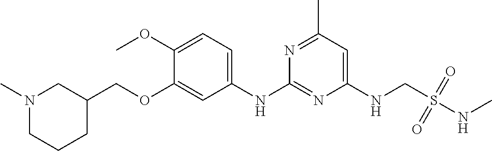 |
| 853 | 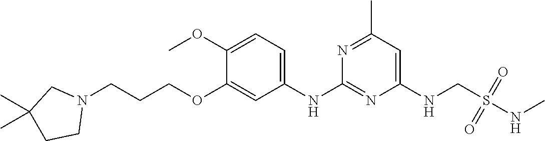 |
| 854 | 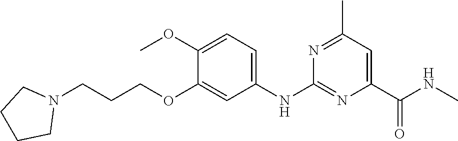 [SMILES: CNC(=O)c1cc(C)nc(Nc2ccc(OC)c(OCCCN3CCCC3)c2)n1] |
| 855 | 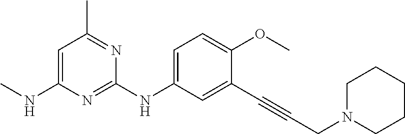 [SMILES: CNc1cc(C)nc(Nc2ccc(OC)c(C#CCN3CCCCC3)c2)n1] |
| 856 | 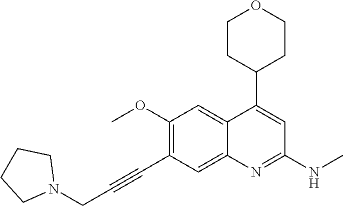 [SMILES: CNc1cc(C2CCOCC2)c2cc(OC)c(C#CCN3CCCC3)cc2n1] |
| 857 | 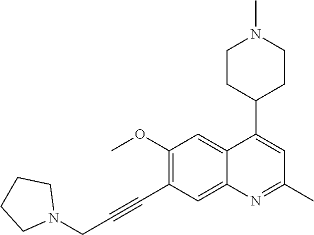 [SMILES: COc1cc2c(C3CCN(C)CC3)cc(C)nc2cc1C#CCN1CCCC1] |
| 858 | 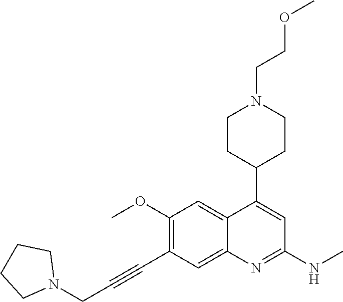 [SMILES: CNc1cc(C2CCN(CCOC)CC2)c2cc(OC)c(C#CCN3CCCC3)cc2n1] |
| 859 | 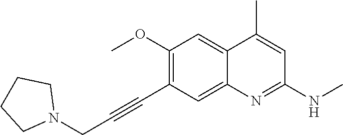 [SMILES: CNc1cc(C)c2cc(OC)c(C#CCN3CCCC3)cc2n1] |
| 860 | 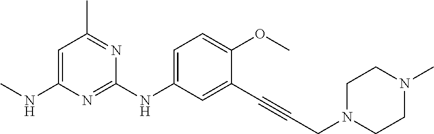 |
| 861 | 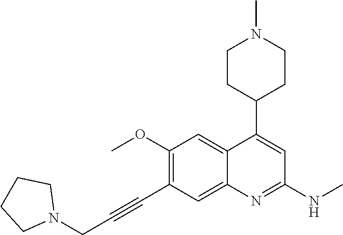 [SMILES: CNc1cc(C2CCN(C)CC2)c2cc(OC)c(C#CCN3CCCC3)cc2n1] |
| 862 | 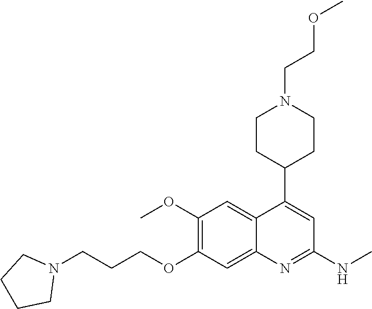 [SMILES: CNc1cc(C2CCN(CCOC)CC2)c2cc(OC)c(OCCCN3CCCC3)cc2n1] |
| 863 | 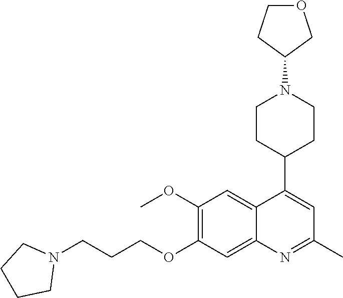 [SMILES: COc1cc2c(C3CCN([C@@H]4CCOC4)CC3)cc(C)nc2cc1OCCCN1CCCC1] |
| 864 | 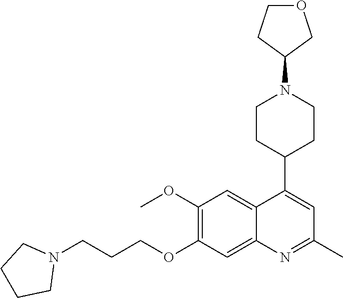 [SMILES: COc1cc2c(C3CCN([C@H]4CCOC4)CC3)cc(C)nc2cc1OCCCN1CCCC1] |
| 865 | 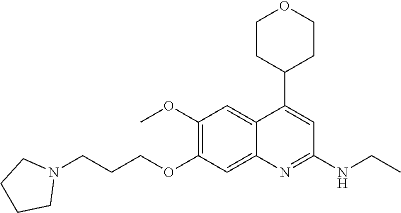 [SMILES: CCNc1cc(C2CCOCC2)c2cc(OC)c(OCCCN3CCCC3)cc2n1] |
| 866 | 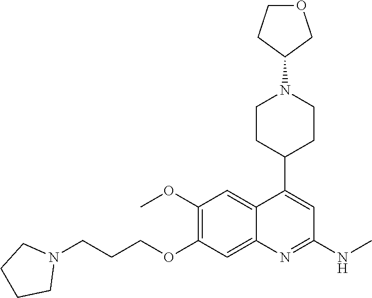 [SMILES: CNc1cc(C2CCN([C@@H]3CCOC3)CC2)c2cc(OC)c(OCCCN3CCCC3)cc2n1] |
| 867 | 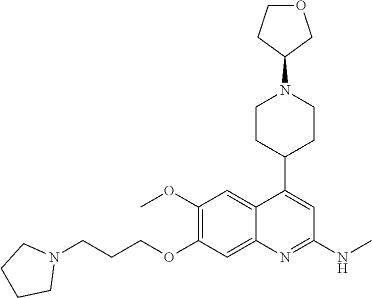 [SMILES: CNc1cc(C2CCN([C@H]3CCOC3)CC2)c2cc(OC)c(OCCCN3CCCC3)cc2n1] |
| 868 | 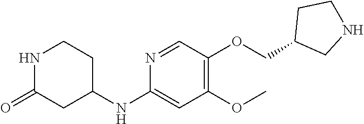 [SMILES: COc1cc(NC2CCNC(=O)C2)ncc1OC[C@@H]1CCNC1] |
| 869 | 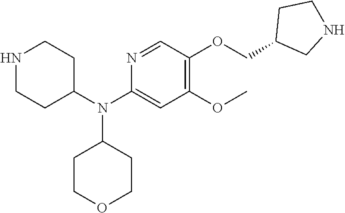 [SMILES: COc1cc(N(C2CCNCC2)C2CCOCC2)ncc1OC[C@@H]1CCNC1] |
| 870 | 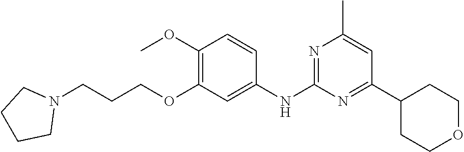 [SMILES: COc1ccc(Nc2nc(C)cc(C3CCOCC3)n2)cc1OCCCN1CCCC1] |
| 871 | 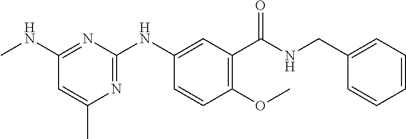 [SMILES: CNc1cc(C)nc(Nc2ccc(OC)c(C(=O)NCc3ccccc3)c2)n1] |
| 872 | 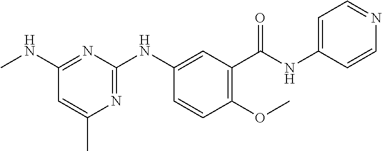 [SMILES: CNc1cc(C)nc(Nc2ccc(OC)c(C(=O)Nc3ccncc3)c2)n1] |
| 873 | 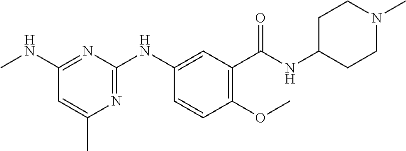 |
| 874 | 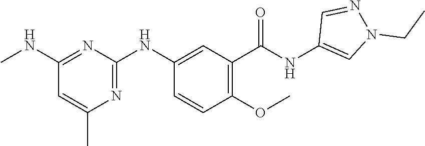 [SMILES: CCn1cc(NC(=O)c2cc(Nc3nc(C)cc(NC)n3)ccc2OC)cn1] |
| 875 | 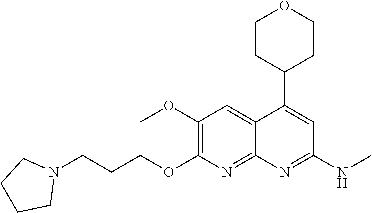 [SMILES: CNc1cc(C2CCOCC2)c2cc(OC)c(OCCCN3CCCC3)nc2n1] |
| 876 | 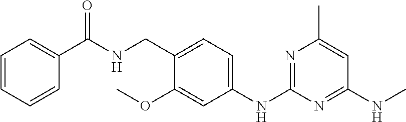 [SMILES: CNc1cc(C)nc(Nc2ccc(CNC(=O)c3ccccc3)c(OC)c2)n1] |
| 877 | 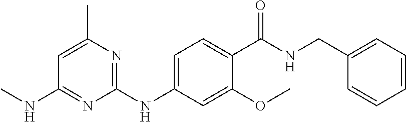 [SMILES: CNc1cc(C)nc(Nc2ccc(C(=O)NCc3ccccc3)c(OC)c2)n1] |
| 878 | 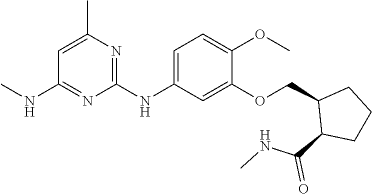 [SMILES: CNC(=O)[C@@H]1CCC[C@@H]1COc1cc(Nc2nc(C)cc(NC)n2)ccc1OC] |
| 879 | 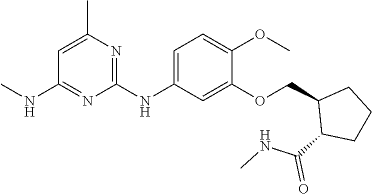 [SMILES: CNC(=O)[C@H]1CCC[C@@H]1COc1cc(Nc2nc(C)cc(NC)n2)ccc1OC] |
| 881 | 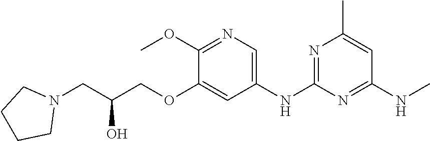 [SMILES: CNc1cc(C)nc(Nc2cnc(OC)c(OC[C@@H](O)CN3CCCC3)c2)n1] |
| 882 | 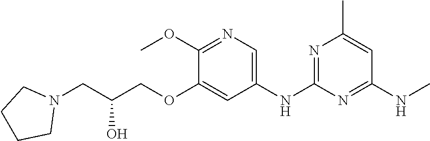 [SMILES: CNc1cc(C)nc(Nc2cnc(OC)c(OC[C@H](O)CN3CCCC3)c2)n1] |
| 883 | 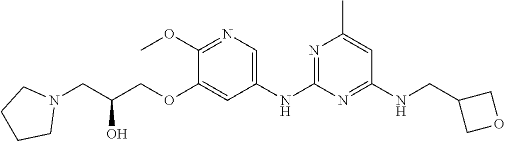 [SMILES: COc1ncc(Nc2nc(C)cc(NCC3COC3)n2)cc1OC[C@@H](O)CN1CCCC1] |
| 884 | 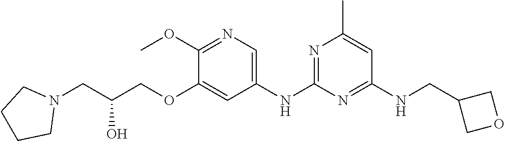 [SMILES: COc1ncc(Nc2nc(C)cc(NCC3COC3)n2)cc1OC[C@H](O)CN1CCCC1] |
| 885 | 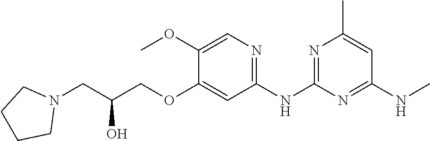 [SMILES: CNc1cc(C)nc(Nc2cc(OC[C@@H](O)CN3CCCC3)c(OC)cn2)n1] |
| 886 | 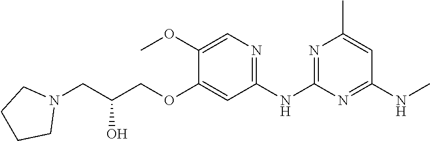 [SMILES: CNc1cc(C)nc(Nc2cc(OC[C@H](O)CN3CCCC3)c(OC)cn2)n1] |
| 887 | 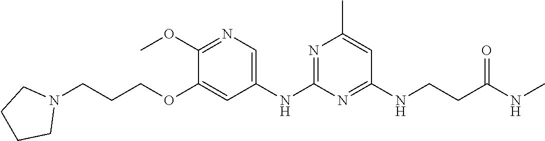 [SMILES: CNC(=O)CCNc1cc(C)nc(Nc2cnc(OC)c(OCCCN3CCCC3)c2)n1] |
| 888 | 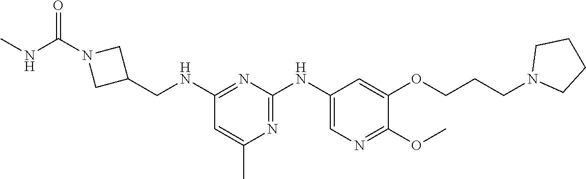 [SMILES: CNC(=O)N1CC(CNc2cc(C)nc(Nc3cnc(OC)c(OCCCN4CCCC4)c3)n2)C1] |
| 890 | 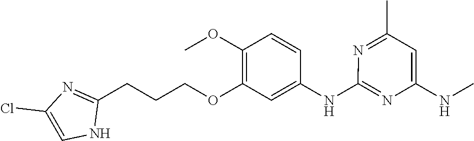 [SMILES: CNc1cc(C)nc(Nc2ccc(OC)c(OCCCc3nc(Cl)c[nH]3)c2)n1] |
| 891 | 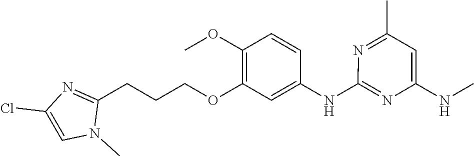 [SMILES: CNc1cc(C)nc(Nc2ccc(OC)c(OCCCc3nc(Cl)cn3C)c2)n1] |
| 892 | 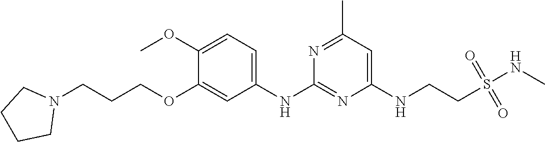 [SMILES: CNS(=O)(=O)CCNc1cc(C)nc(Nc2ccc(OC)c(OCCCN3CCCC3)c2)n1] |
| 893 | 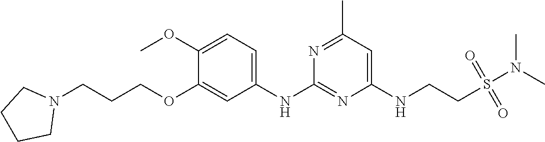 [SMILES: COc1ccc(Nc2nc(C)cc(NCCS(=O)(=O)N(C)C)n2)cc1OCCCN1CCCC1] |
| 894 | 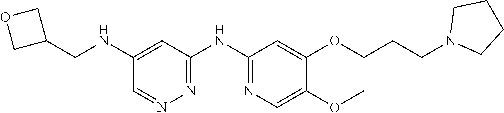 [SMILES: COc1cnc(Nc2cc(NCC3COC3)cnn2)cc1OCCCN1CCCC1] |
| 895 | 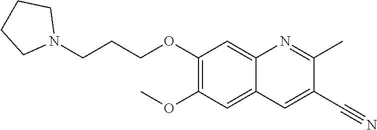 [SMILES: COc1cc2cc(C#N)c(C)nc2cc1OCCCN1CCCC1] |
| 896 | 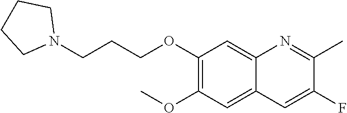 [SMILES: COc1cc2cc(F)c(C)nc2cc1OCCCN1CCCC1] |
| 897 | 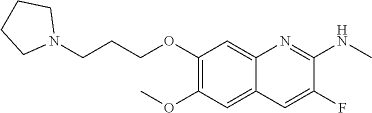 [SMILES: CNc1nc2cc(OCCCN3CCCC3)c(OC)cc2cc1F] |
| 898 | 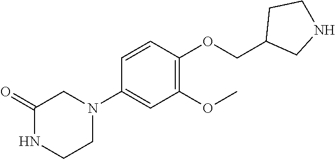 [SMILES: COc1cc(N2CCNC(=O)C2)ccc1OCC1CCNC1] |
| 899 | 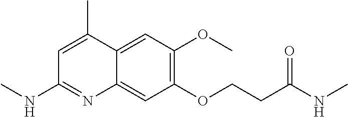 |
| 900 | 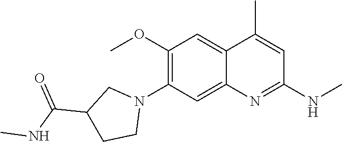 |
| 901 | 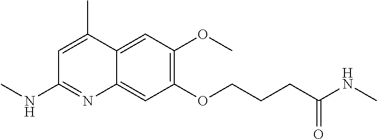 |
| 902 | 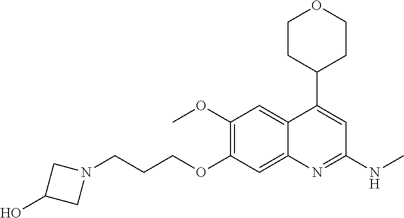 [SMILES: CNc1cc(C2CCOCC2)c2cc(OC)c(OCCCN3CC(O)C3)cc2n1] |
| 903 | 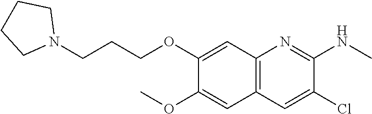 [SMILES: CNc1nc2cc(OCCCN3CCCC3)c(OC)cc2cc1Cl] |
| 904 | 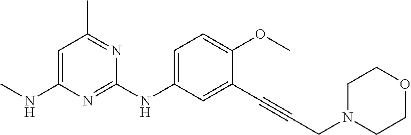 [SMILES: CNc1cc(C)nc(Nc2ccc(OC)c(C#CCN3CCOCC3)c2)n1] |
| 905 | 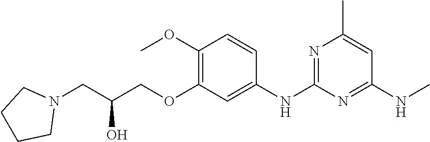 [SMILES: CNc1cc(C)nc(Nc2ccc(OC)c(OC[C@@H](O)CN3CCCC3)c2)n1] |
| 906 | 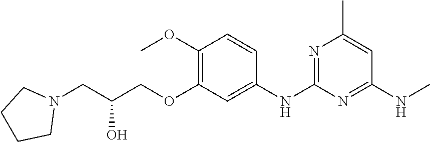 [SMILES: CNc1cc(C)nc(Nc2ccc(OC)c(OC[C@H](O)CN3CCCC3)c2)n1] |
| 907 | 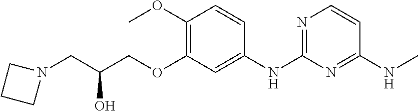 [SMILES: CNc1ccnc(Nc2ccc(OC)c(OC[C@@H](O)CN3CCC3)c2)n1] |
| 908 | 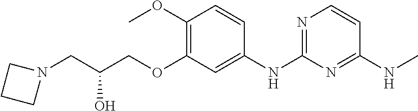 [SMILES: CNc1ccnc(Nc2ccc(OC)c(OC[C@H](O)CN3CCC3)c2)n1] |
| 909 | 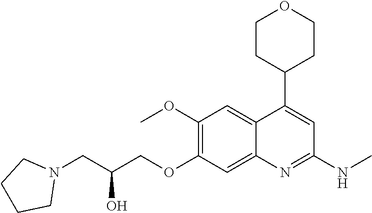 [SMILES: CNc1cc(C2CCOCC2)c2cc(OC)c(OC[C@@H](O)CN3CCCC3)cc2n1] |
| 910 | 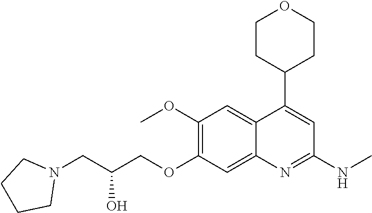 [SMILES: CNc1cc(C2CCOCC2)c2cc(OC)c(OC[C@H](O)CN3CCCC3)cc2n1] |
| 911 | 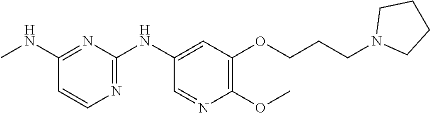 [SMILES: CNc1ccnc(Nc2cnc(OC)c(OCCCN3CCCC3)c2)n1] |
| 912 | 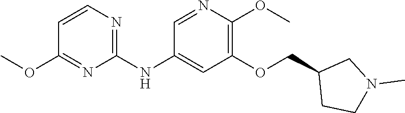 |
| 913 | 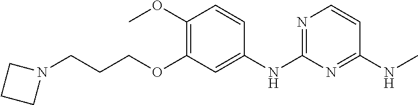 [SMILES: CNc1ccnc(Nc2ccc(OC)c(OCCCN3CCC3)c2)n1] |
| 914 | 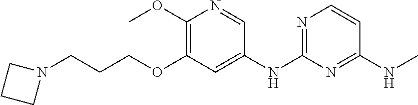 [SMILES: CNc1ccnc(Nc2cnc(OC)c(OCCCN3CCC3)c2)n1] |
| 915 | 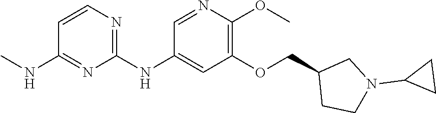 [SMILES: CNc1ccnc(Nc2cnc(OC)c(OC[C@@H]3CCN(C4CC4)C3)c2)n1] |
| 916 | 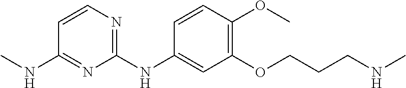 |
| 917 | 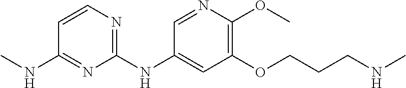 |
| 918 | 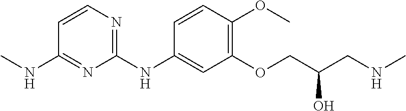 |
| 919 | 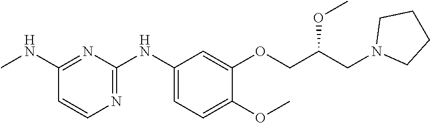 [SMILES: CNc1ccnc(Nc2ccc(OC)c(OC[C@@H](CN3CCCC3)OC)c2)n1] |
| 920 | 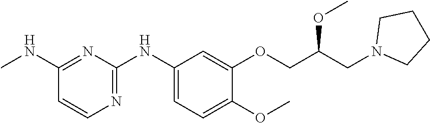 [SMILES: CNc1ccnc(Nc2ccc(OC)c(OC[C@H](CN3CCCC3)OC)c2)n1] |
| 921 | 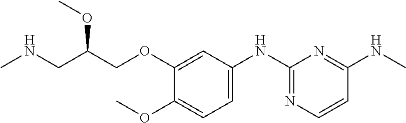 |
| 922 | 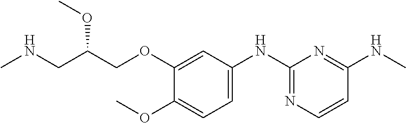 |
| 927 | 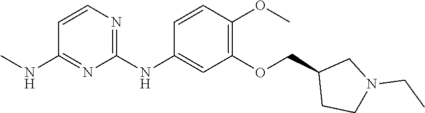 [SMILES: CCN1CC[C@@H](COc2cc(Nc3nccc(NC)n3)ccc2OC)C1] |
| 928 | 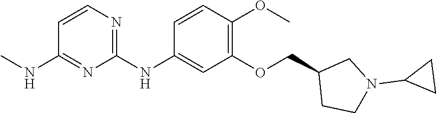 [SMILES: CNc1ccnc(Nc2ccc(OC)c(OC[C@@H]3CCN(C4CC4)C3)c2)n1] |
| 929 | 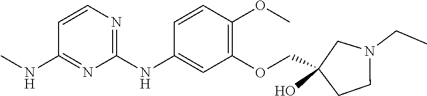 [SMILES: CCN1CC[C@](O)(COc2cc(Nc3nccc(NC)n3)ccc2OC)C1] |
| 930 | 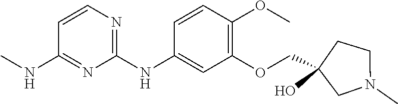 |
| 931 | 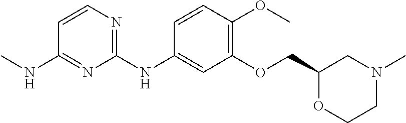 |
| 932 | 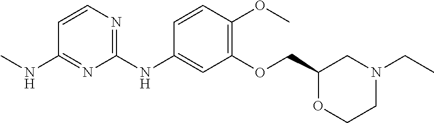 [SMILES: CCN1CCO[C@@H](COc2cc(Nc3nccc(NC)n3)ccc2OC)C1] |
| 933 | 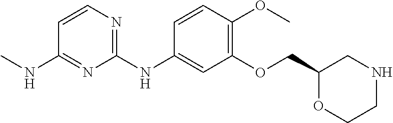 [SMILES: CNc1ccnc(Nc2ccc(OC)c(OC[C@H]3CNCCO3)c2)n1] |
| 934 | 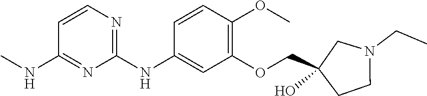 [SMILES: CCN1CC[C@@](O)(COc2cc(Nc3nccc(NC)n3)ccc2OC)C1] |
| 935 | 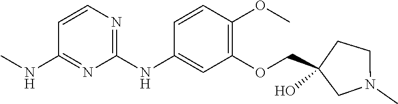 |
| 936 | 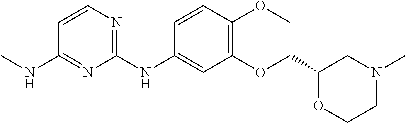 |
| 937 | 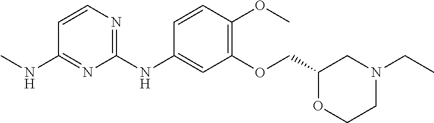 [SMILES: CCN1CCO[C@H](COc2cc(Nc3nccc(NC)n3)ccc2OC)C1] |
| 938 | 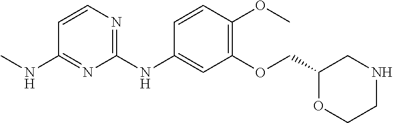 [SMILES: CNc1ccnc(Nc2ccc(OC)c(OC[C@@H]3CNCCO3)c2)n1] |
| 939 | 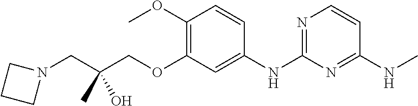 [SMILES: CNc1ccnc(Nc2ccc(OC)c(OC[C@](C)(O)CN3CCC3)c2)n1] |
| 940 | 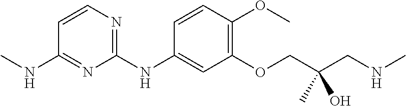 |
| 941 | 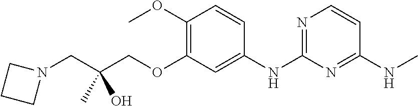 [SMILES: CNc1ccnc(Nc2ccc(OC)c(OC[C@@](C)(O)CN3CCC3)c2)n1] |
| 942 | 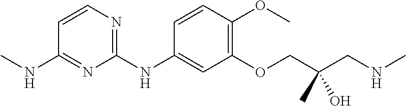 |
| 943 | 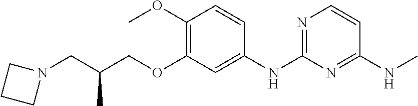 [SMILES: CNc1ccnc(Nc2ccc(OC)c(OC[C@@H](C)CN3CCC3)c2)n1] |
| 944 | 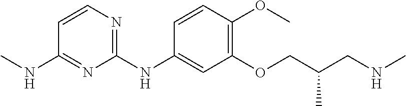 |
| 945 | 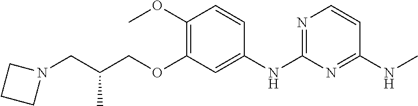 [SMILES: CNc1ccnc(Nc2ccc(OC)c(OC[C@H](C)CN3CCC3)c2)n1] |
| 946 | 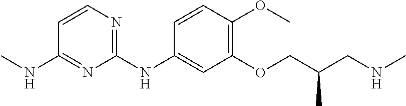 |
| 947 | 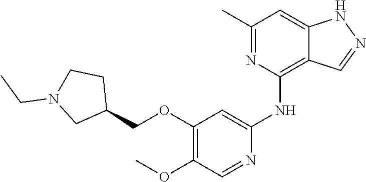 [SMILES: CCN1CC[C@@H](COc2cc(Nc3nc(C)cc4[nH]ncc34)ncc2OC)C1] |
| 948 | 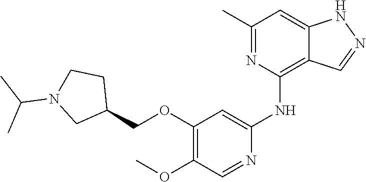 [SMILES: COc1cnc(Nc2nc(C)cc3[nH]ncc23)cc1OC[C@@H]1CCN(C(C)C)C1] |
| 949 | 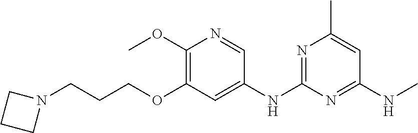 [SMILES: CNc1cc(C)nc(Nc2cnc(OC)c(OCCCN3CCC3)c2)n1] |
| 950 | 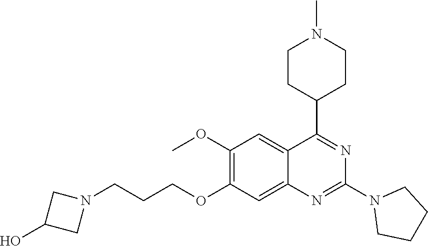 [SMILES: COc1cc2c(C3CCN(C)CC3)nc(N3CCCC3)nc2cc1OCCCN1CC(O)C1] |
| 951 | 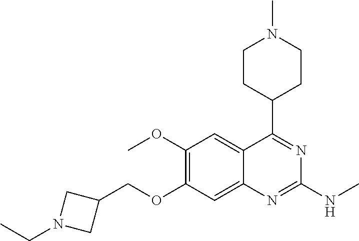 [SMILES: CCN1CC(COc2ccc(/C(=N\C(=N/C)NC)C3CCN(C)CC3)cc2OC)C1] |
| 961 | 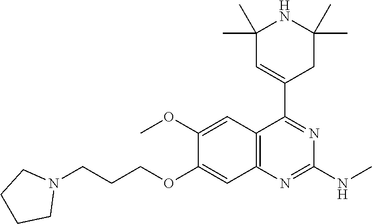 [SMILES: C/N=C(\N=C(\C1=CC(C)(C)NC(C)(C)C1)c1ccc(OCCCN2CCCC2)c(OC)c1)NC] |
| 962 | 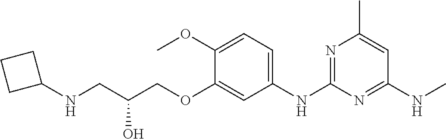 [SMILES: CNc1cc(C)nc(Nc2ccc(OC)c(OC[C@H](O)CNC3CCC3)c2)n1] |
| 963 | 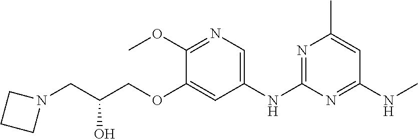 [SMILES: CNc1cc(C)nc(Nc2cnc(OC)c(OC[C@H](O)CN3CCC3)c2)n1] |
| 964 | 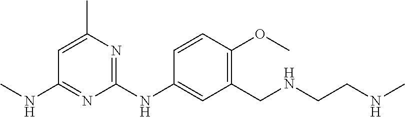 |
| 965 | 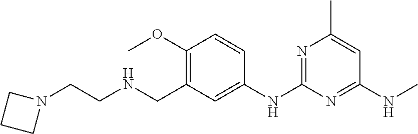 [SMILES: CNc1cc(C)nc(Nc2ccc(OC)c(CNCCN3CCC3)c2)n1] |
| 966 | 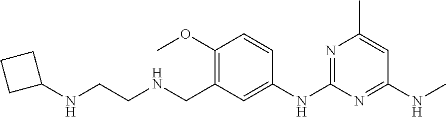 [SMILES: CNc1cc(C)nc(Nc2ccc(OC)c(CNCCNC3CCC3)c2)n1] |
| 967 | 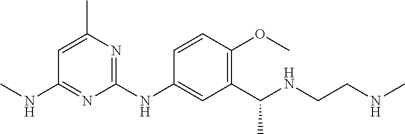 |
| 968 | 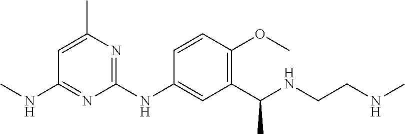 |
| 969 | 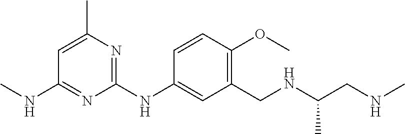 |
| 970 | 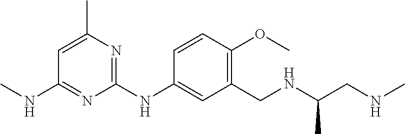 |
| 971 | 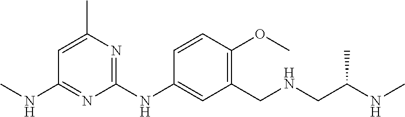 |
| 972 | 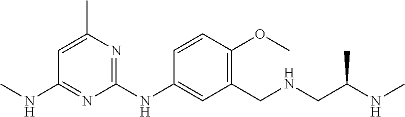 |
| 974 | 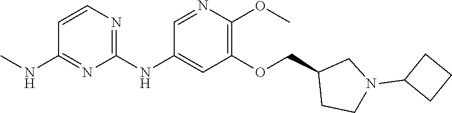 [SMILES: CNc1ccnc(Nc2cnc(OC)c(OC[C@@H]3CCN(C4CCC4)C3)c2)n1] |
| 975 | 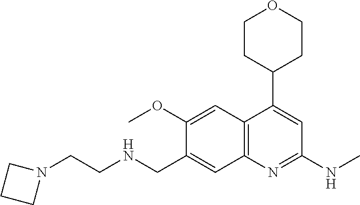 [SMILES: C/N=C(\C=C(/c1ccc(CNCCN2CCC2)c(OC)c1)C1CCOCC1)NC] |
| 976 | 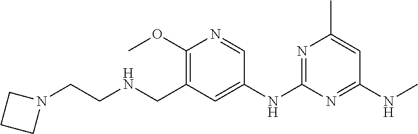 [SMILES: CNc1cc(C)nc(Nc2cnc(OC)c(CNCCN3CCC3)c2)n1] |
| 977 | 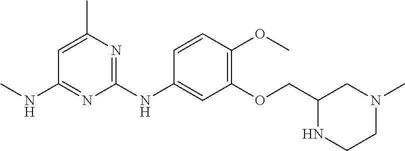 |
| 983 | 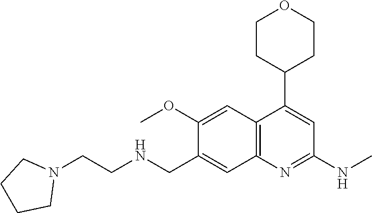 [SMILES: C/N=C(\C=C(/c1ccc(CNCCN2CCCC2)c(OC)c1)C1CCOCC1)NC] |
| 985 | 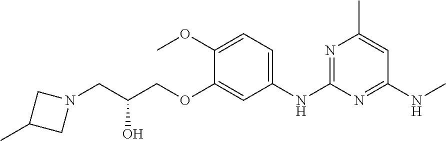 |
| 986 | 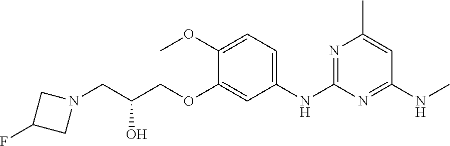 [SMILES: CNc1cc(C)nc(Nc2ccc(OC)c(OC[C@H](O)CN3CC(F)C3)c2)n1] |
| 989 | 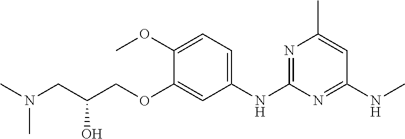 |
| 990 | 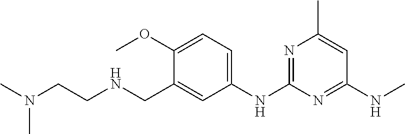 |
| 991 | 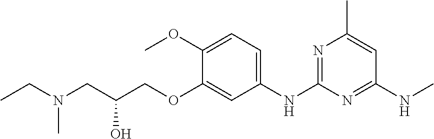 [SMILES: CCN(C)C[C@@H](O)COc1cc(Nc2nc(C)cc(NC)n2)ccc1OC] |
| 992 | 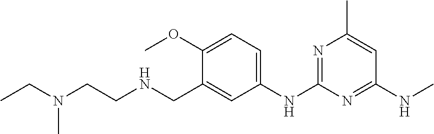 [SMILES: CCN(C)CCNCc1cc(Nc2nc(C)cc(NC)n2)ccc1OC] |
| 993 | 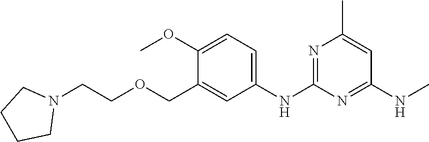 [SMILES: CNc1cc(C)nc(Nc2ccc(OC)c(COCCN3CCCC3)c2)n1] |
| 994 | 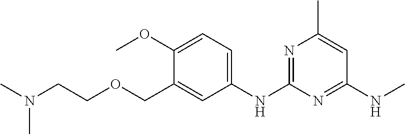 |
| 997 | 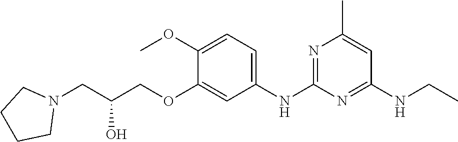 [SMILES: CCNc1cc(C)nc(Nc2ccc(OC)c(OC[C@H](O)CN3CCCC3)c2)n1] |
| 998 | 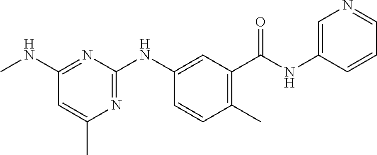 [SMILES: CNc1cc(C)nc(Nc2ccc(C)c(C(=O)Nc3cccnc3)c2)n1] |
| 999 | 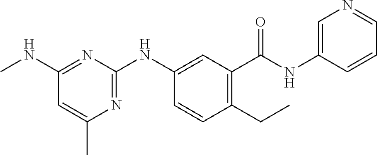 [SMILES: CCc1ccc(Nc2nc(C)cc(NC)n2)cc1C(=O)Nc1cccnc1] |
| 1000 | 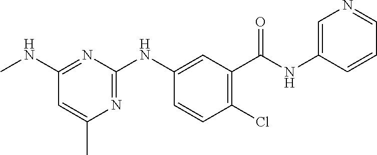 [SMILES: CNc1cc(C)nc(Nc2ccc(Cl)c(C(=O)Nc3cccnc3)c2)n1] |
| 1001 | 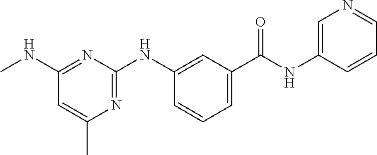 [SMILES: CNc1cc(C)nc(Nc2cccc(C(=O)Nc3cccnc3)c2)n1] |
| 1002 | 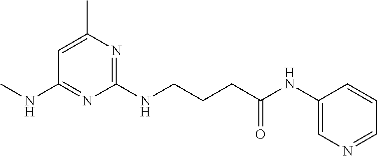 [SMILES: CNc1cc(C)nc(NCCCC(=O)Nc2cccnc2)n1] |
| 1004 | 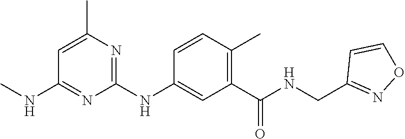 [SMILES: CNc1cc(C)nc(Nc2ccc(C)c(C(=O)NCc3ccon3)c2)n1] |
| 1005 | 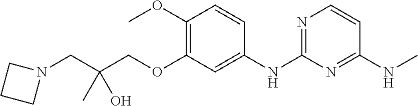 [SMILES: CNc1ccnc(Nc2ccc(OC)c(OCC(C)(O)CN3CCC3)c2)n1] |
| 1006 | 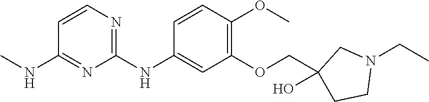 [SMILES: CCN1CCC(O)(COc2cc(Nc3nccc(NC)n3)ccc2OC)C1] |
| 1007 | 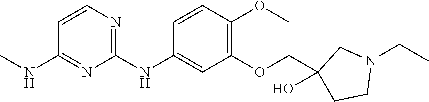 [SMILES: CCN1CCC(O)(COc2cc(Nc3nccc(NC)n3)ccc2OC)C1] |
| 1008 | 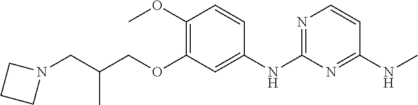 [SMILES: CNc1ccnc(Nc2ccc(OC)c(OCC(C)CN3CCC3)c2)n1] |
| 1009 | 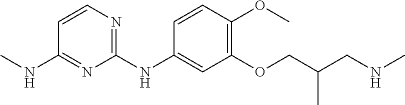 |
| 1010 | 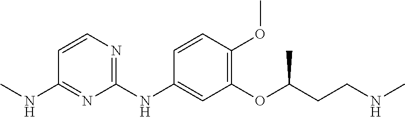 |
| 1011 | 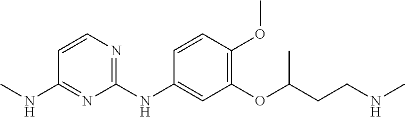 |
| 1012 | 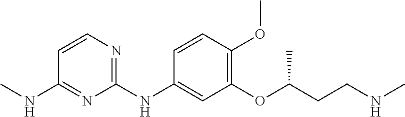 |
| 1013 | 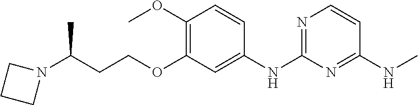 [SMILES: CNc1ccnc(Nc2ccc(OC)c(OCC[C@H](C)N3CCC3)c2)n1] |
| 1014 | 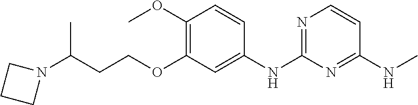 [SMILES: CNc1ccnc(Nc2ccc(OC)c(OCCC(C)N3CCC3)c2)n1] |
| 1015 | 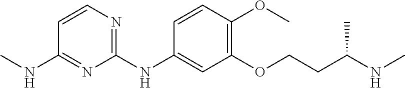 |
| 1016 | 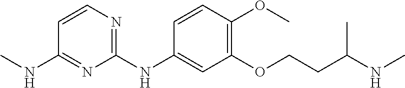 |
| 1017 | 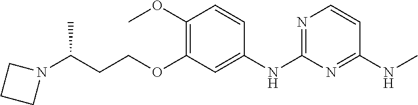 [SMILES: CNc1ccnc(Nc2ccc(OC)c(OCC[C@@H](C)N3CCC3)c2)n1] |
| 1018 | 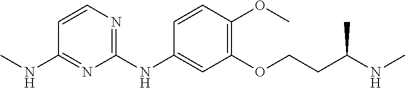 |
| 1019 | 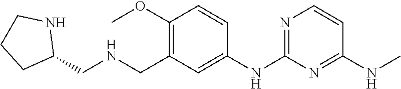 [SMILES: CNc1ccnc(Nc2ccc(OC)c(CNC[C@@H]3CCCN3)c2)n1] |
| 1020 | 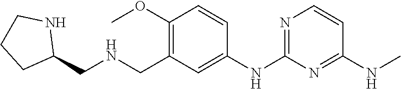 [SMILES: CNc1ccnc(Nc2ccc(OC)c(CNC[C@H]3CCCN3)c2)n1] |
| 1021 | 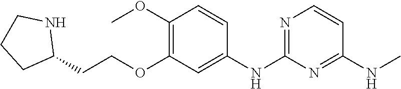 [SMILES: CNc1ccnc(Nc2ccc(OC)c(OCC[C@@H]3CCCN3)c2)n1] |
| 1022 | 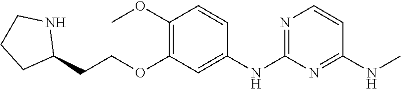 [SMILES: CNc1ccnc(Nc2ccc(OC)c(OCC[C@H]3CCCN3)c2)n1] |
| 1023 | 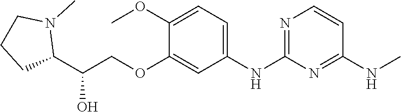 [SMILES: CNc1ccnc(Nc2ccc(OC)c(OC[C@H](O)[C@@H]3CCCN3C)c2)n1] |
| 1024 | 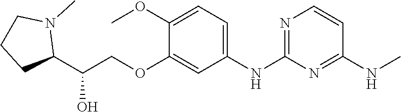 [SMILES: CNc1ccnc(Nc2ccc(OC)c(OC[C@H](O)[C@H]3CCCN3C)c2)n1] |
| 1025 | 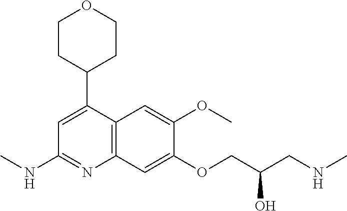 |
| 1026 | 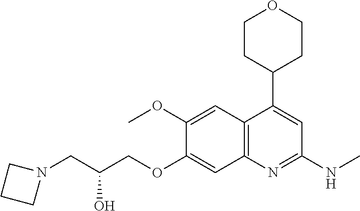 [SMILES: C/N=C(\C=C(/c1ccc(OC[C@H](O)CN2CCC2)c(OC)c1)C1CCOCC1)NC] |
| 1027 | 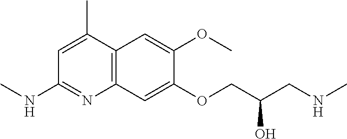 |
| 1028 | 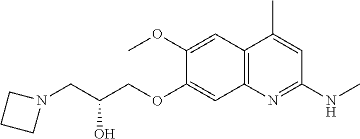 [SMILES: C/N=C(\C=C(\C)c1ccc(OC[C@H](O)CN2CCC2)c(OC)c1)NC] |
| 1029 | 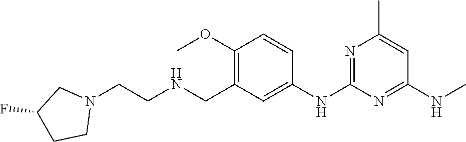 [SMILES: CNc1cc(C)nc(Nc2ccc(OC)c(CNCCN3CC[C@H](F)C3)c2)n1] |
| 1030 | 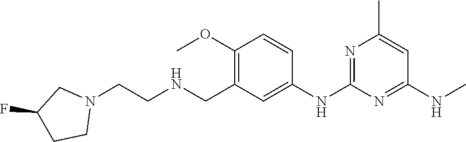 [SMILES: CNc1cc(C)nc(Nc2ccc(OC)c(CNCCN3CC[C@@H](F)C3)c2)n1] |
| 1031 | 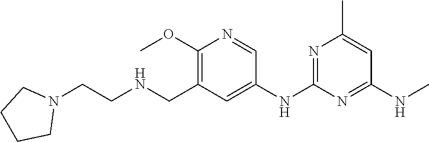 [SMILES: CNc1cc(C)nc(Nc2cnc(OC)c(CNCCN3CCCC3)c2)n1] |
| 1032 | 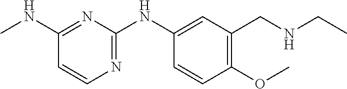 [SMILES: CCNCc1cc(Nc2nccc(NC)n2)ccc1OC] |
| 1033 | 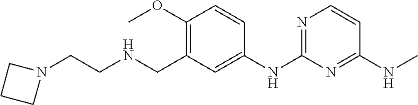 [SMILES: C=C/C(CNCCN1CCC1)=C(\C=C/CNc1nccc(NC)n1)OC] |
| 1034 | 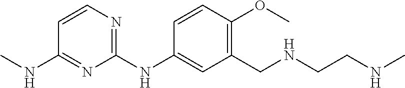 |
| 1035 | 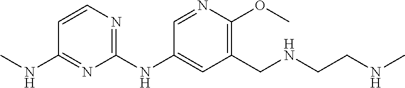 |
| 1036 | 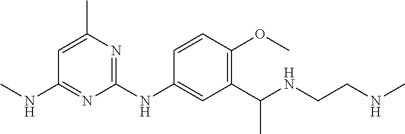 |
| 1037 | 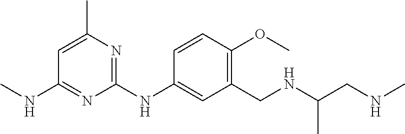 |
| 1038 | 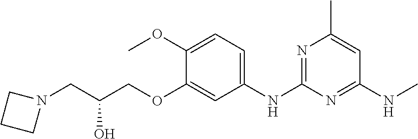 [SMILES: C\C=C(OC[C@H](O)CN1CCC1)/C(=C\C=C\Nc1nc(C)cc(NC)n1)OC] |
| 1039 | 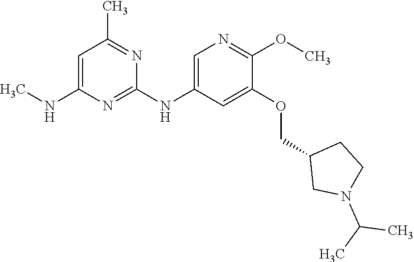 [SMILES: CNc1cc(C)nc(Nc2cnc(OC)c(OC[C@@H]3CCN(C(C)C)C3)c2)n1] |
| 1040 | 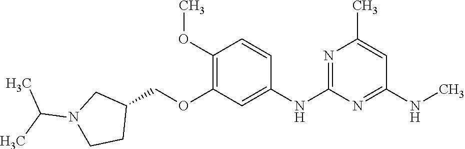 [SMILES: CNc1cc(C)nc(Nc2ccc(OC)c(OC[C@@H]3CCN(C(C)C)C3)c2)n1] |
| 1041 | 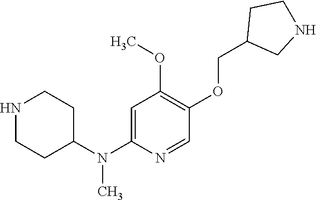 [SMILES: COc1cc(N(C)C2CCNCC2)ncc1OCC1CCNC1] |
| 1042 | 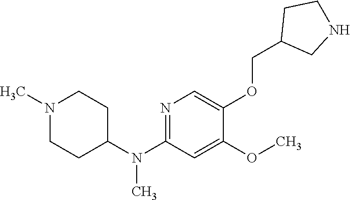 [SMILES: COc1cc(N(C)C2CCN(C)CC2)ncc1OCC1CCNC1] |

TABLE 1E

| Cmpd. | |
|---|---|
| No. | Structure |
| 1043 | 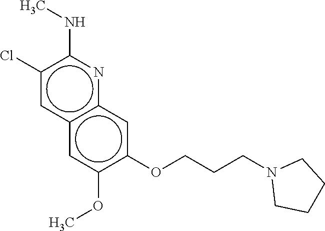 |
| 1044 | 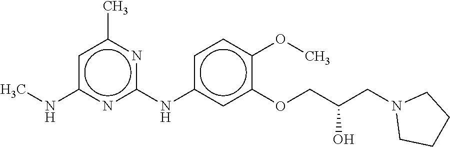 |
| 1045 | 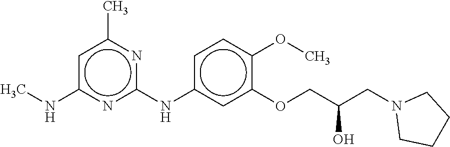 |
| 1046 | 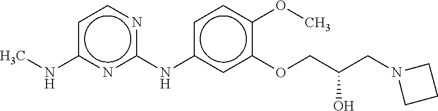 |
| 1047 | 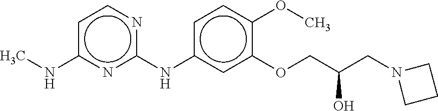 |
| 1048 | 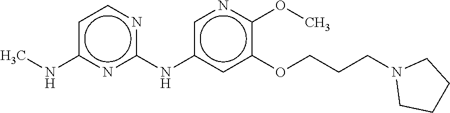 |
| 1049 | 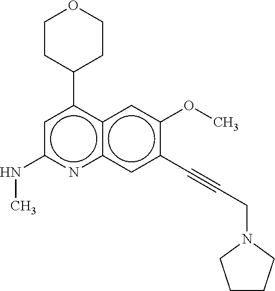 |
| 1050 | 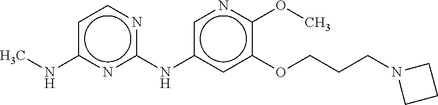 |
| 1051 | 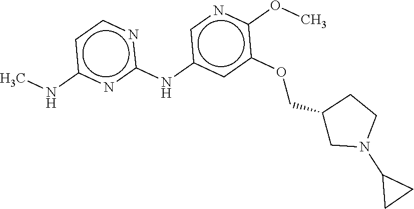 |
| 1052 | 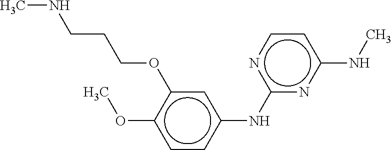 |
| 1053 | 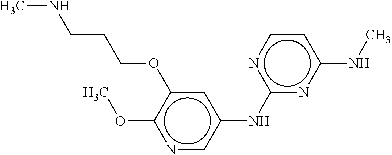 |
| 1054 | 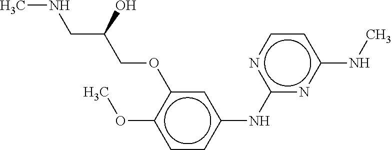 |
| 1055 | 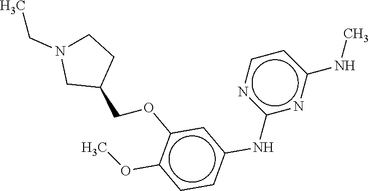 |
| 1056 | 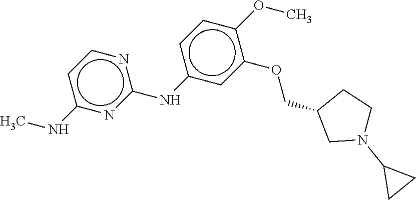 |
| 1057 | 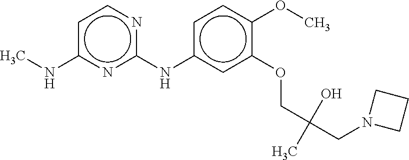 |
| 1058 | 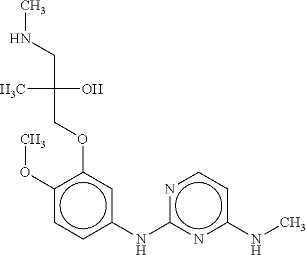 |
| 1059 | 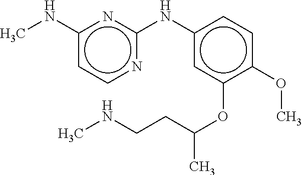 |
| 1060 | 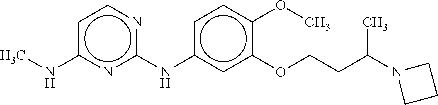 |
| 1061 | 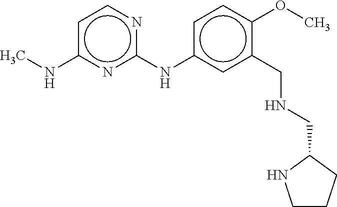 |
| 1062 | 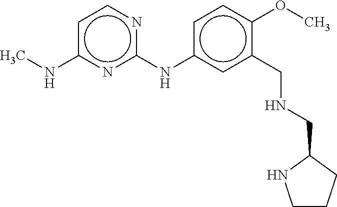 |
| 1063 | 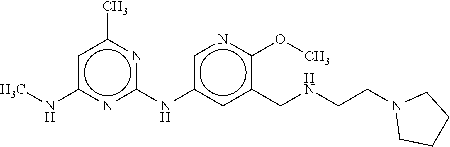 |
| 1064 | 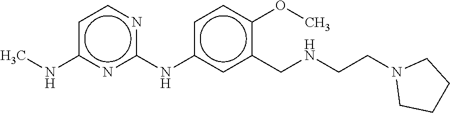 |
| 1065 | 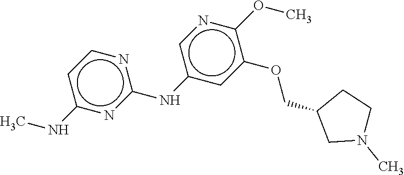 |
| 1066 | 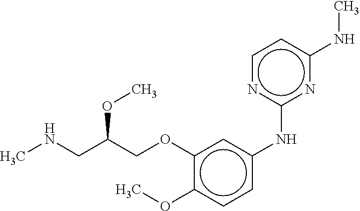 |
| 1067 | 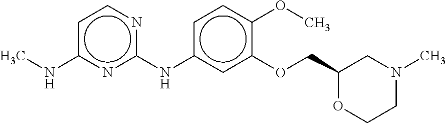 |
| 1068 | 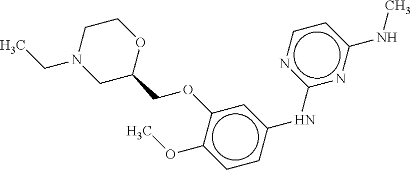 |
| 1069 | 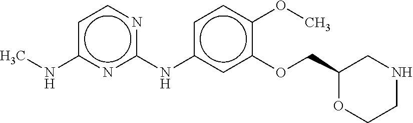 |
| 1070 | 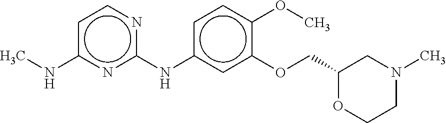 |
| 1071 | 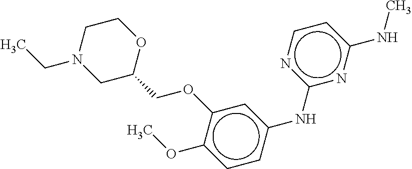 |
| 1072 | 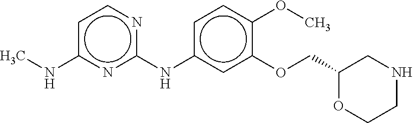 |
| 1073 | 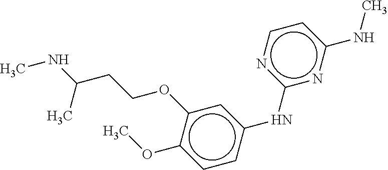 |
| 1074 | 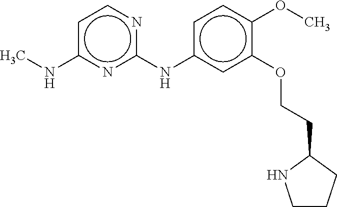 |
| 1075 | 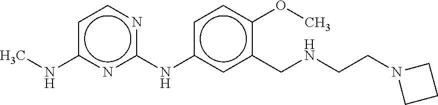 |
| 1076 | 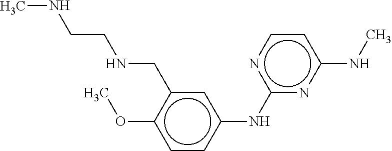 |
| 1077 | 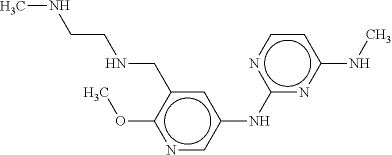 |
| 1078 | 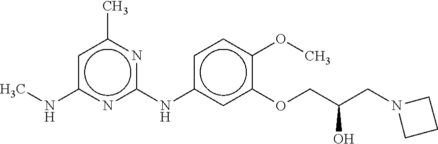 |
| 1079 | 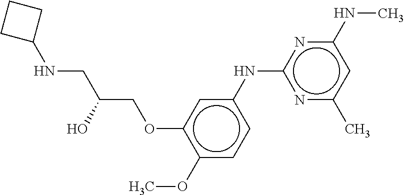 |
| 1080 | 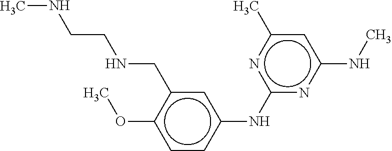 |
| 1081 | 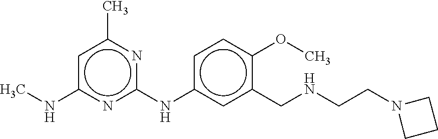 |
| 1082 | 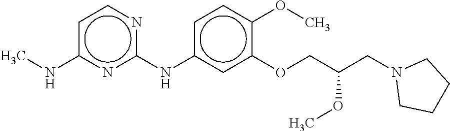 |
| 1083 | 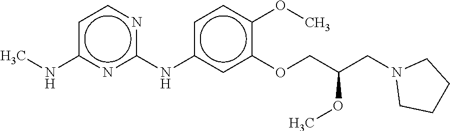 |
| 1084 | 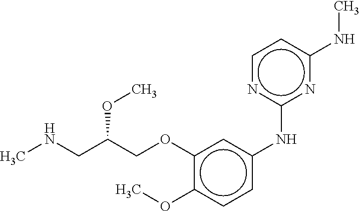 |
| 1085 | 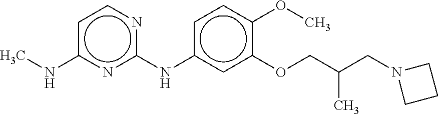 |
| 1086 | 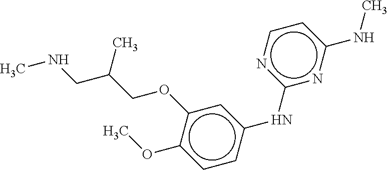 |
| 1087 | 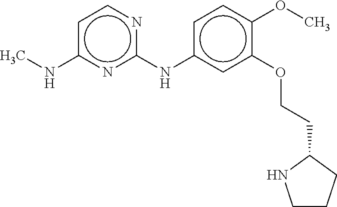 |
| 1088 | 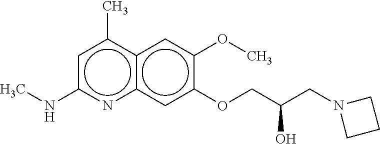 |
| 1089 | 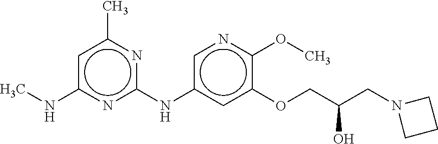 |
| 1090 | 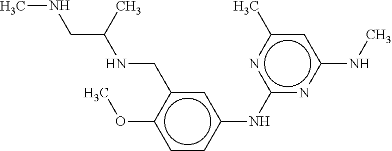 |
| 1091 | 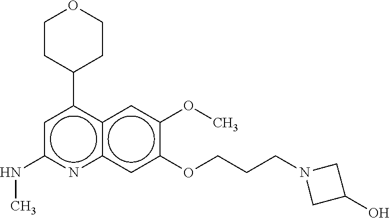 |
| 1092 | 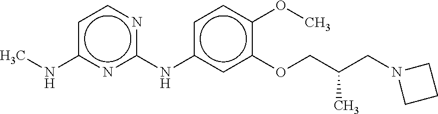 |
| 1093 | 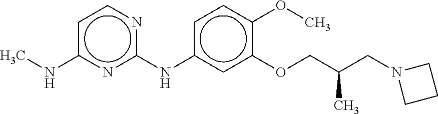 |
| 1094 | 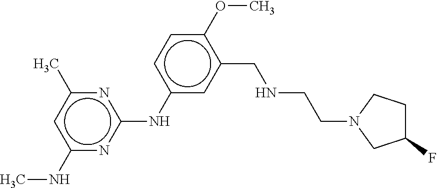 |
| 1095 | 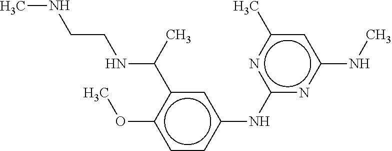 |
| 1096 | 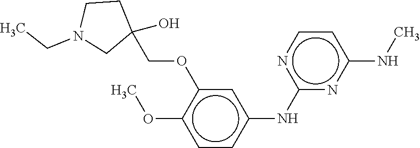 |
| 1097 | 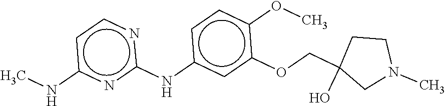 |
| 1098 | 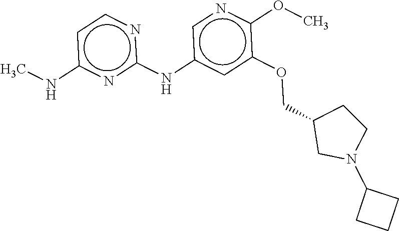 |
| 1099 | 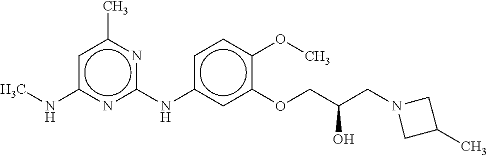 |
| 1100 | 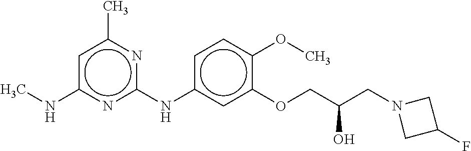 |
| 1101 | 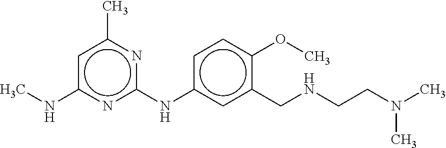 |
| 1102 | 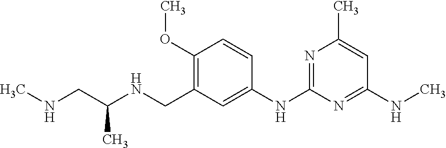 |
| 1103 | 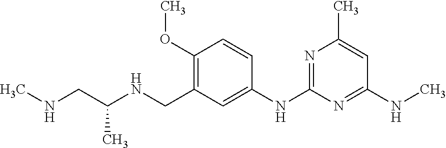 |
| 1104 | 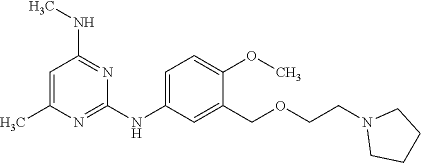 [SMILES: CNc1cc(C)nc(Nc2ccc(OC)c(COCCN3CCCC3)c2)n1] |
| 1105 | 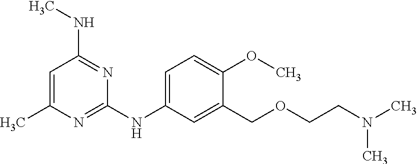 |
| 1106 | 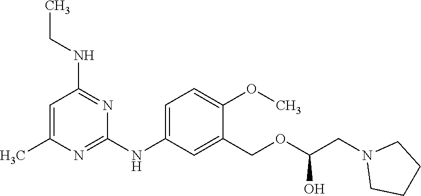 [SMILES: CCNc1cc(C)nc(Nc2ccc(OC)c(CO[C@H](O)CN3CCCC3)c2)n1] |
| 1107 | 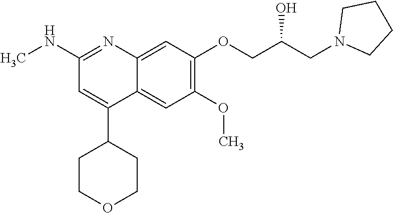 [SMILES: CNc1cc(C2CCOCC2)c2cc(OC)c(OC[C@H](O)CN3CCCC3)cc2n1] |
| 1108 | 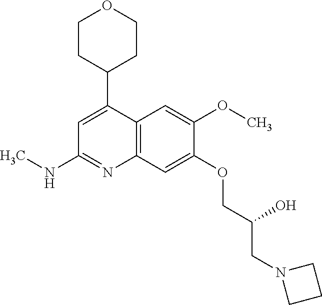 [SMILES: CNc1cc(C2CCOCC2)c2cc(OC)c(OC[C@H](O)CN3CCC3)cc2n1] |
| 1109 | 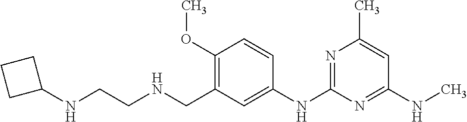 [SMILES: CNc1cc(C)nc(Nc2ccc(OC)c(CNCCNC3CCC3)c2)n1] |
| 1110 | 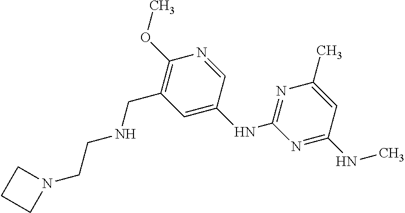 [SMILES: CNc1cc(C)nc(Nc2cnc(OC)c(CNCCN3CCC3)c2)n1] |
| 1111 | 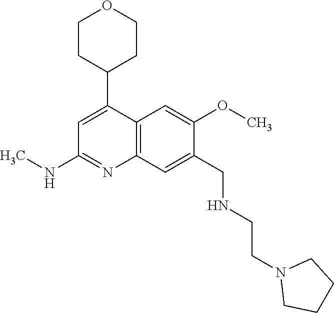 [SMILES: CNc1cc(C2CCOCC2)c2cc(OC)c(CNCCN3CCCC3)cc2n1] |
| 1112 | 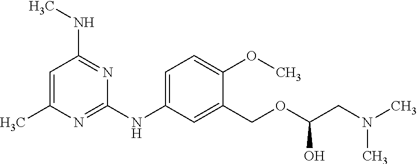 |
| 1113 | 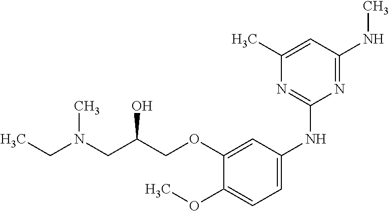 [SMILES: CCN(C)C[C@@H](O)COc1cc(Nc2nc(C)cc(NC)n2)ccc1OC] |
| 1114 | 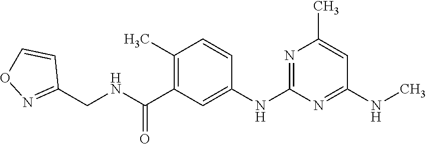 [SMILES: CNc1cc(C)nc(Nc2ccc(C)c(C(=O)NCc3ccon3)c2)n1] |
| 1115 | 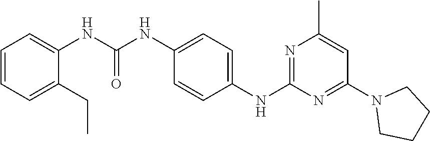 [SMILES: CCc1ccccc1NC(=O)Nc1ccc(Nc2nc(C)cc(N3CCCC3)n2)cc1] |
| 1116 | 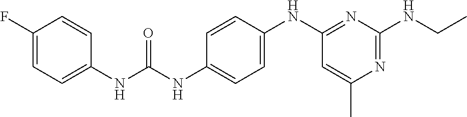 [SMILES: CCNc1nc(C)cc(Nc2ccc(NC(=O)Nc3ccc(F)cc3)cc2)n1] |
| 1117 | 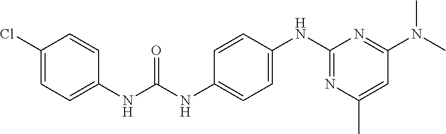 [SMILES: Cc1cc(N(C)C)nc(Nc2ccc(NC(=O)Nc3ccc(Cl)cc3)cc2)n1] |
| 1118 | 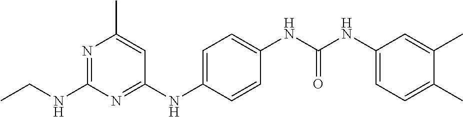 [SMILES: CCNc1nc(C)cc(Nc2ccc(NC(=O)Nc3ccc(C)c(C)c3)cc2)n1] |

TABLE 2

| Compound | |
|---|---|
| No. | Structure |
| A1 | 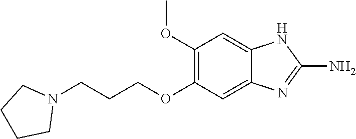 [SMILES: COc1cc2[nH]c(N)nc2cc1OCCCN1CCCC1] |
| A2 | 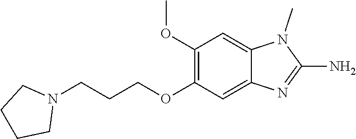 [SMILES: COc1cc2c(cc1OCCCN1CCCC1)nc(N)n2C] |
| A3 | 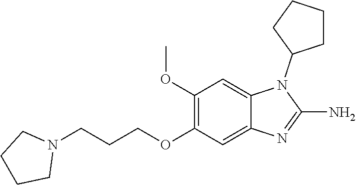 [SMILES: COc1cc2c(cc1OCCCN1CCCC1)nc(N)n2C1CCCC1] |
| A4 | 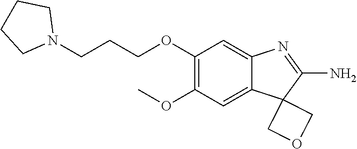 [SMILES: COc1cc2c(cc1OCCCN1CCCC1)N=C(N)C21COC1] |
| A5 | 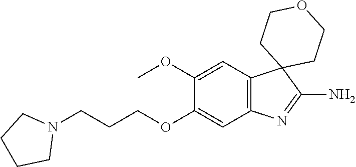 [SMILES: COc1cc2c(cc1OCCCN1CCCC1)N=C(N)C21CCOCC1] |
| A6 | 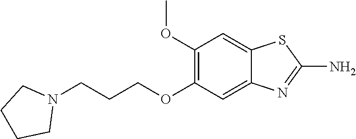 [SMILES: COc1cc2sc(N)nc2cc1OCCCN1CCCC1] |
| A7 | 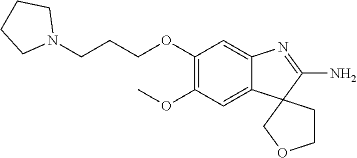 [SMILES: COc1cc2c(cc1OCCCN1CCCC1)N=C(N)C21CCOC1] |
| A8 | 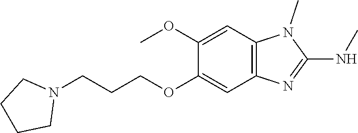 [SMILES: CNc1nc2cc(OCCCN3CCCC3)c(OC)cc2n1C] |
| A9 | 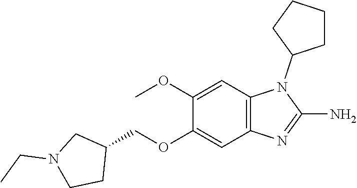 [SMILES: CCN1CC[C@@H](COc2cc3nc(N)n(C4CCCC4)c3cc2OC)C1] |
| A10 | 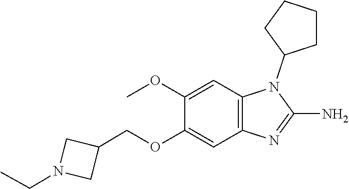 [SMILES: CCN1CC(COc2cc3nc(N)n(C4CCCC4)c3cc2OC)C1] |
| A11 | 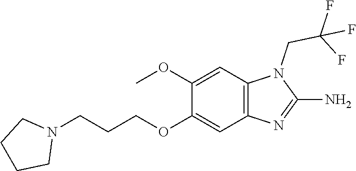 [SMILES: COc1cc2c(cc1OCCCN1CCCC1)nc(N)n2CC(F)(F)F] |
| A12 | 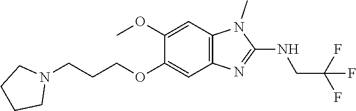 [SMILES: COc1cc2c(cc1OCCCN1CCCC1)nc(NCC(F)(F)F)n2C] |
| A13 | 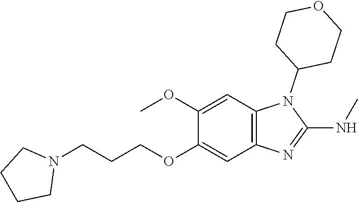 [SMILES: CNc1nc2cc(OCCCN3CCCC3)c(OC)cc2n1C1CCOCC1] |
| A14 | 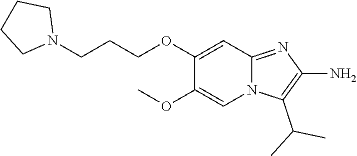 [SMILES: COc1cn2c(C(C)C)c(N)nc2cc1OCCCN1CCCC1] |
| A15 | 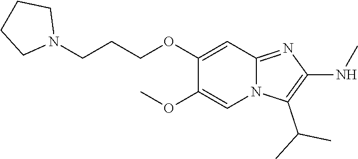 [SMILES: CNc1nc2cc(OCCCN3CCCC3)c(OC)cn2c1C(C)C] |
| A16 | 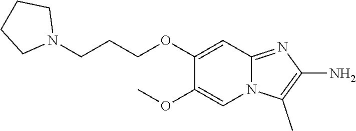 [SMILES: COc1cn2c(C)c(N)nc2cc1OCCCN1CCCC1] |
| A17 | 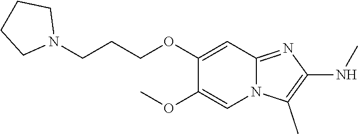 [SMILES: CNc1nc2cc(OCCCN3CCCC3)c(OC)cn2c1C] |
| A18 | 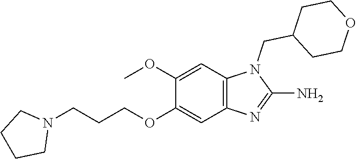 [SMILES: COc1cc2c(cc1OCCCN1CCCC1)nc(N)n2CC1CCOCC1] |
| A19 | 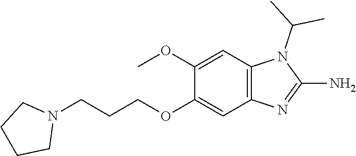 [SMILES: COc1cc2c(cc1OCCCN1CCCC1)nc(N)n2C(C)C] |
| A20 | 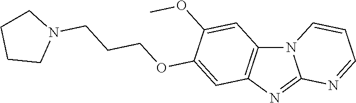 [SMILES: COc1cc2c(cc1OCCCN1CCCC1)nc1ncccn12] |
| A21 | 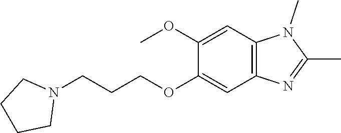 [SMILES: COc1cc2c(cc1OCCCN1CCCC1)nc(C)n2C] |
| A22 | 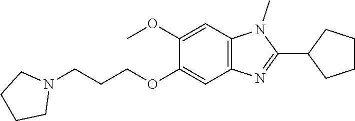 [SMILES: COc1cc2c(cc1OCCCN1CCCC1)nc(C1CCCC1)n2C] |
| A23 | 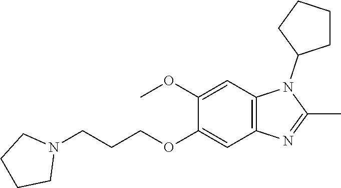 [SMILES: COc1cc2c(cc1OCCCN1CCCC1)nc(C)n2C1CCCC1] |
| A24 | 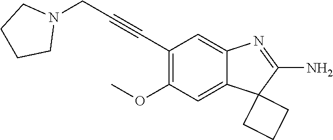 [SMILES: COc1cc2c(cc1C#CCN1CCCC1)N=C(N)C21CCC1] |
| A25 | 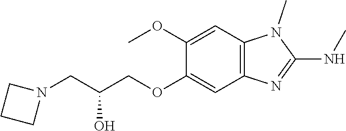 [SMILES: CNc1nc2cc(OC[C@H](O)CN3CCC3)c(OC)cc2n1C] |
| A26 | 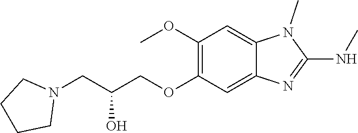 [SMILES: CNc1nc2cc(OC[C@H](O)CN3CCCC3)c(OC)cc2n1C] |
| A27 | 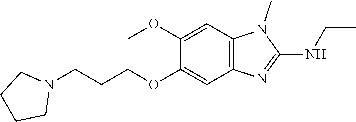 [SMILES: CCNc1nc2cc(OCCCN3CCCC3)c(OC)cc2n1C] |
| A28 | 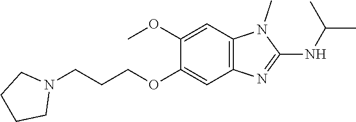 [SMILES: COc1cc2c(cc1OCCCN1CCCC1)nc(NC(C)C)n2C] |
| A29 | 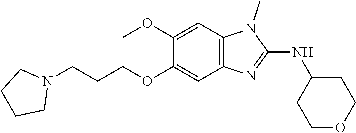 [SMILES: COc1cc2c(cc1OCCCN1CCCC1)nc(NC1CCOCC1)n2C] |
| A30 | 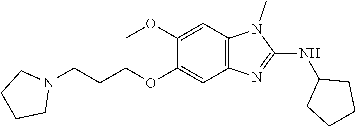 [SMILES: COc1cc2c(cc1OCCCN1CCCC1)nc(NC1CCCC1)n2C] |
| A31 | 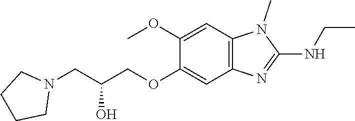 [SMILES: CCNc1nc2cc(OC[C@H](O)CN3CCCC3)c(OC)cc2n1C] |
| A32 | 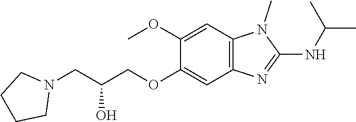 [SMILES: COc1cc2c(cc1OC[C@H](O)CN1CCCC1)nc(NC(C)C)n2C] |
| A33 | 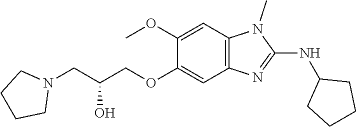 [SMILES: COc1cc2c(cc1OC[C@H](O)CN1CCCC1)nc(NC1CCCC1)n2C] |
| A34 | 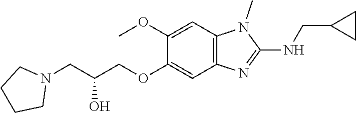 [SMILES: COc1cc2c(cc1OC[C@H](O)CN1CCCC1)nc(NCC1CC1)n2C] |
| A35 | 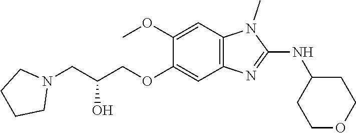 [SMILES: COc1cc2c(cc1OC[C@H](O)CN1CCCC1)nc(NC1CCOCC1)n2C] |
| A36 | 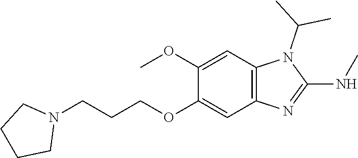 [SMILES: CNc1nc2cc(OCCCN3CCCC3)c(OC)cc2n1C(C)C] |
| A37 | 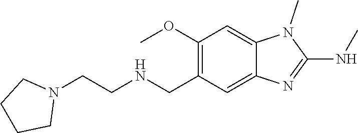 [SMILES: CNc1nc2cc(CNCCN3CCCC3)c(OC)cc2n1C] |
| A38 | 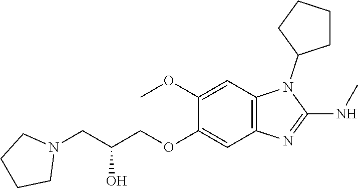 [SMILES: CNc1nc2cc(OC[C@H](O)CN3CCCC3)c(OC)cc2n1C1CCCC1] |
| A39 | 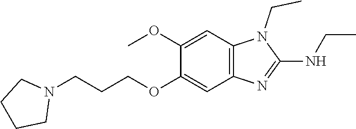 [SMILES: CCNc1nc2cc(OCCCN3CCCC3)c(OC)cc2n1CC] |
| A40 | 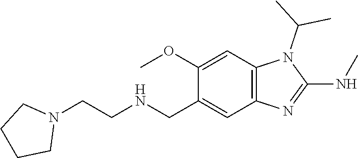 [SMILES: CNc1nc2cc(CNCCN3CCCC3)c(OC)cc2n1C(C)C] |
| A41 | 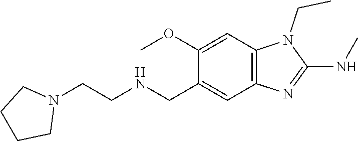 [SMILES: CCn1c(NC)nc2cc(CNCCN3CCCC3)c(OC)cc21] |
| A42 | 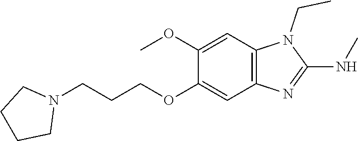 [SMILES: CCn1c(NC)nc2cc(OCCCN3CCCC3)c(OC)cc21] |
| A43 | 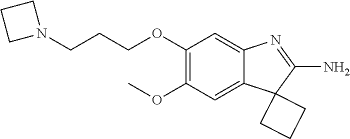 [SMILES: COc1cc2c(cc1OCCCN1CCC1)N=C(N)C21CCC1] |
| A44 | 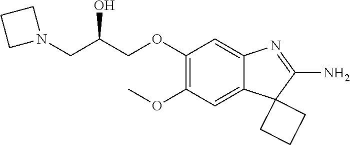 [SMILES: COc1cc2c(cc1OC[C@H](O)CN1CCC1)N=C(N)C21CCC1] |
| A45 | 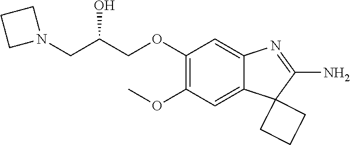 [SMILES: COc1cc2c(cc1OC[C@@H](O)CN1CCC1)N=C(N)C21CCC1] |
| A46 | 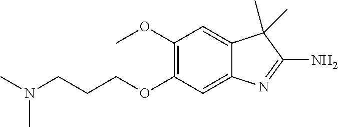 [SMILES: COc1cc2c(cc1OCCCN(C)C)N=C(N)C2(C)C] |
| A47 | 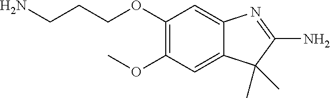 [SMILES: COc1cc2c(cc1OCCCN)N=C(N)C2(C)C] |
| A48 | 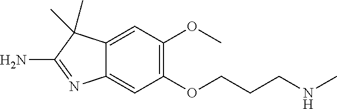 [SMILES: CNCCCOc1cc2c(cc1OC)C(C)(C)C(N)=N2] |
| A49 | 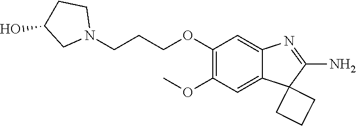 [SMILES: COc1cc2c(cc1OCCCN1CC[C@@H](O)C1)N=C(N)C21CCC1] |
| A50 | 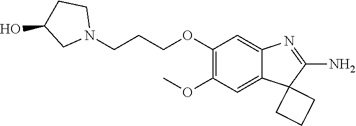 [SMILES: COc1cc2c(cc1OCCCN1CC[C@H](O)C1)N=C(N)C21CCC1] |
| A51 | 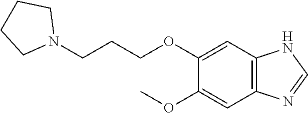 [SMILES: COc1cc2nc[nH]c2cc1OCCCN1CCCC1] |
| A52 | 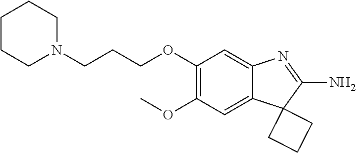 [SMILES: COc1cc2c(cc1OCCCN1CCCCC1)N=C(N)C21CCC1] |
| A53 | 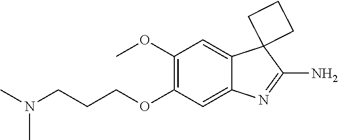 [SMILES: COc1cc2c(cc1OCCCN(C)C)N=C(N)C21CCC1] |
| A54 | 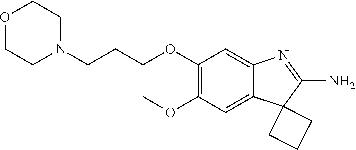 [SMILES: COc1cc2c(cc1OCCCN1CCOCC1)N=C(N)C21CCC1] |
| A55 | 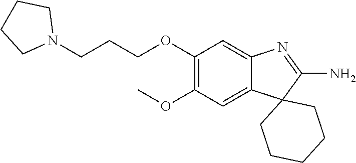 [SMILES: COc1cc2c(cc1OCCCN1CCCC1)N=C(N)C21CCCCC1] |
| A56 | 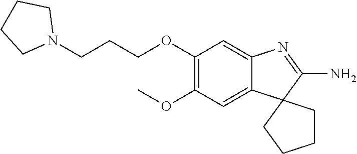 [SMILES: COc1cc2c(cc1OCCCN1CCCC1)N=C(N)C21CCCC1] |
| A57 | 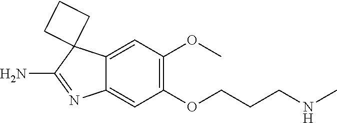 [SMILES: CNCCCOc1cc2c(cc1OC)C1(CCC1)C(N)=N2] |
| A58 | 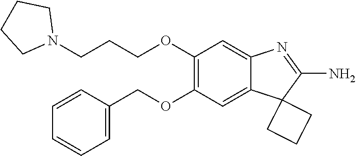 [SMILES: NC1=Nc2cc(OCCCN3CCCC3)c(OCc3ccccc3)cc2C12CCC2] |
| A59 | 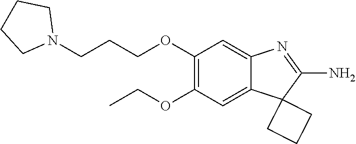 [SMILES: CCOc1cc2c(cc1OCCCN1CCCC1)N=C(N)C21CCC1] |
| A60 | 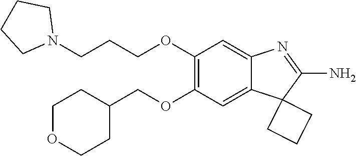 [SMILES: NC1=Nc2cc(OCCCN3CCCC3)c(OCC3CCOCC3)cc2C12CCC2] |
| A61 | 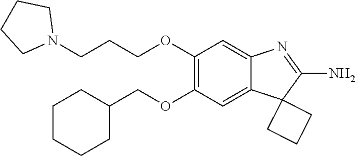 [SMILES: NC1=Nc2cc(OCCCN3CCCC3)c(OCC3CCCCC3)cc2C12CCC2] |
| A62 | 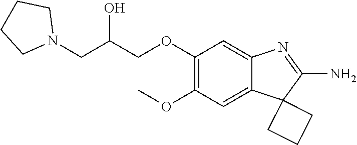 [SMILES: COc1cc2c(cc1OCC(O)CN1CCCC1)N=C(N)C21CCC1] |
| A63 | 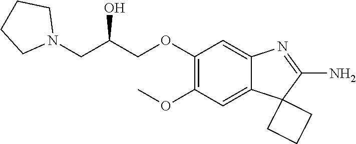 [SMILES: COc1cc2c(cc1OC[C@H](O)CN1CCCC1)N=C(N)C21CCC1] |
| A64 | 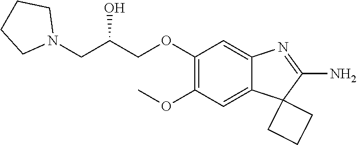 [SMILES: COc1cc2c(cc1OC[C@@H](O)CN1CCCC1)N=C(N)C21CCC1] |
| A65 | 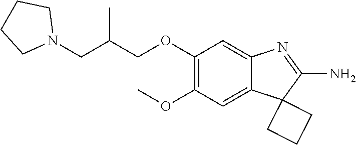 [SMILES: COc1cc2c(cc1OCC(C)CN1CCCC1)N=C(N)C21CCC1] |
| A66 | 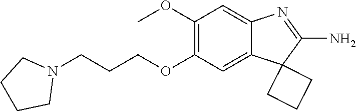 [SMILES: COc1cc2c(cc1OCCCN1CCCC1)C1(CCC1)C(N)=N2] |
| A67 | 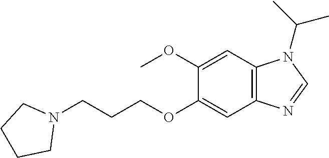 [SMILES: COc1cc2c(cc1OCCCN1CCCC1)ncn2C(C)C] |
| A68 | 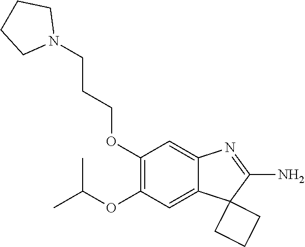 [SMILES: CC(C)Oc1cc2c(cc1OCCCN1CCCC1)N=C(N)C21CCC1] |
| A69 | 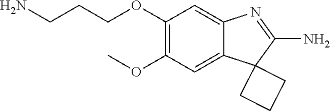 [SMILES: COc1cc2c(cc1OCCCN)N=C(N)C21CCC1] |
| A70 | 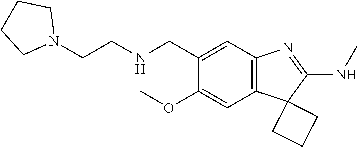 [SMILES: CNC1=Nc2cc(CNCCN3CCCC3)c(OC)cc2C12CCC2] |
| A71 | 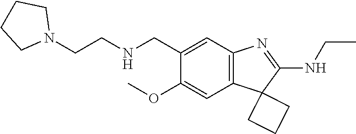 [SMILES: CCNC1=Nc2cc(CNCCN3CCCC3)c(OC)cc2C12CCC2] |
| A72 | 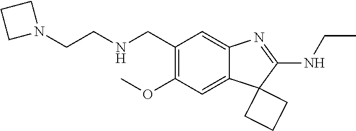 [SMILES: CCNC1=Nc2cc(CNCCN3CCC3)c(OC)cc2C12CCC2] |
| A73 | 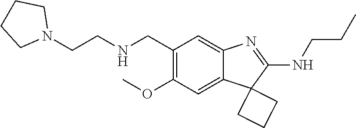 [SMILES: CCCNC1=Nc2cc(CNCCN3CCCC3)c(OC)cc2C12CCC2] |
| A74 | 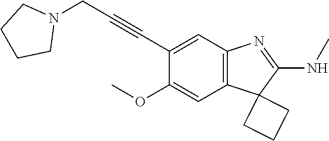 [SMILES: CNC1=Nc2cc(C#CCN3CCCC3)c(OC)cc2C12CCC2] |
| A75 | 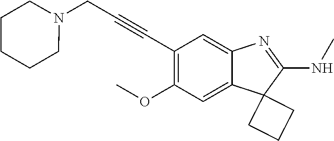 [SMILES: CNC1=Nc2cc(C#CCN3CCCCC3)c(OC)cc2C12CCC2] |
| A76 | 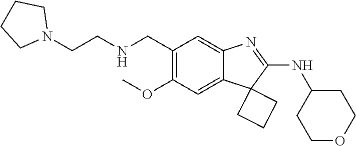 [SMILES: COc1cc2c(cc1CNCCN1CCCC1)N=C(NC1CCOCC1)C21CCC1] |
| A77 | 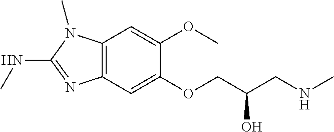 |
| A78 | 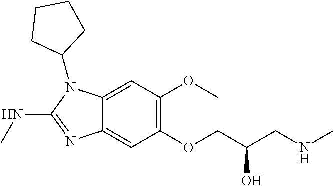 |
| A79 | 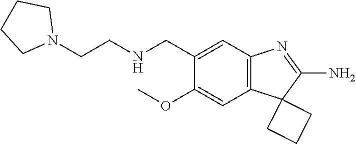 [SMILES: COc1cc2c(cc1CNCCN1CCCC1)N=C(N)C21CCC1] |
| A80 | 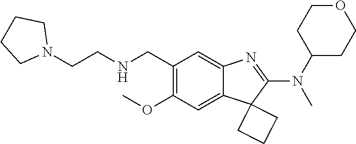 [SMILES: COc1cc2c(cc1CNCCN1CCCC1)N=C(N(C)C1CCOCC1)C21CCC1] |
| A81 | 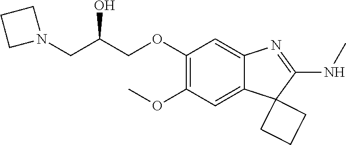 [SMILES: CNC1=Nc2cc(OC[C@H](O)CN3CCC3)c(OC)cc2C12CCC2] |
| A82 | 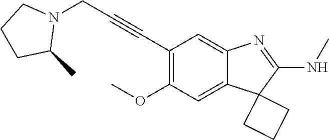 [SMILES: CNC1=Nc2cc(C#CCN3CCC[C@@H]3C)c(OC)cc2C12CCC2] |
| A83 | 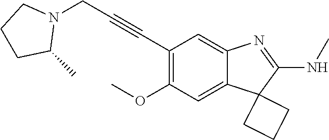 [SMILES: CNC1=Nc2cc(C#CCN3CCC[C@H]3C)c(OC)cc2C12CCC2] |
| A84 | 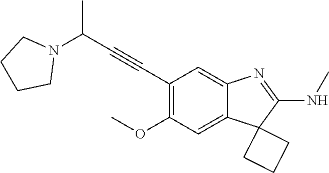 [SMILES: CNC1=Nc2cc(C#CC(C)N3CCCC3)c(OC)cc2C12CCC2] |
| A85 | 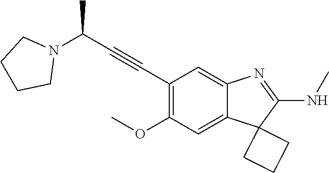 [SMILES: CNC1=Nc2cc(C#C[C@H](C)N3CCCC3)c(OC)cc2C12CCC2] |
| A86 | 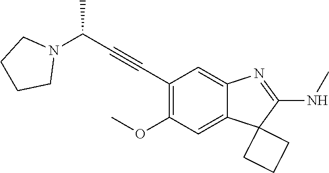 [SMILES: CNC1=Nc2cc(C#C[C@@H](C)N3CCCC3)c(OC)cc2C12CCC2] |
| A87 | 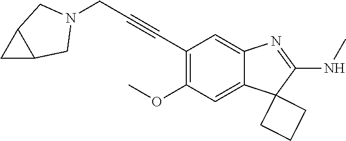 [SMILES: CNC1=Nc2cc(C#CCN3CC4CC4C3)c(OC)cc2C12CCC2] |
| A88 | 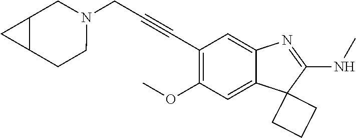 [SMILES: CNC1=Nc2cc(C#CCN3CCC4CC4C3)c(OC)cc2C12CCC2] |
| A89 | 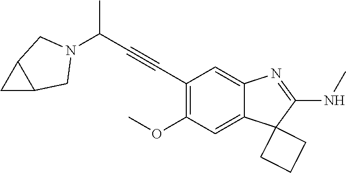 [SMILES: CNC1=Nc2cc(C#CC(C)N3CC4CC4C3)c(OC)cc2C12CCC2] |
| A90 | 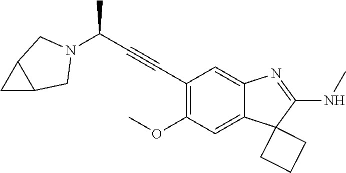 [SMILES: CNC1=Nc2cc(C#C[C@H](C)N3CC4CC4C3)c(OC)cc2C12CCC2] |
| A91 | 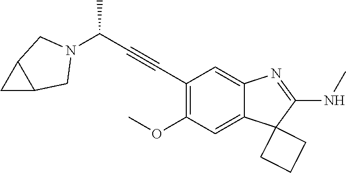 [SMILES: CNC1=Nc2cc(C#C[C@@H](C)N3CC4CC4C3)c(OC)cc2C12CCC2] |
| A92 | 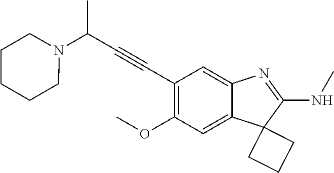 [SMILES: CNC1=Nc2cc(C#CC(C)N3CCCCC3)c(OC)cc2C12CCC2] |
| A93 | 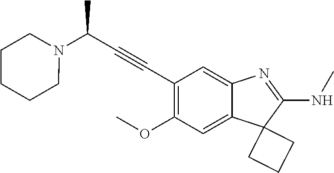 [SMILES: CNC1=Nc2cc(C#C[C@H](C)N3CCCCC3)c(OC)cc2C12CCC2] |
| A94 | 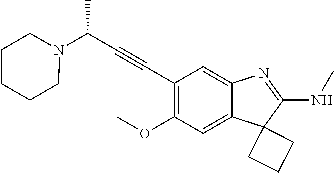 [SMILES: CNC1=Nc2cc(C#C[C@@H](C)N3CCCCC3)c(OC)cc2C12CCC2] |
| A95 | 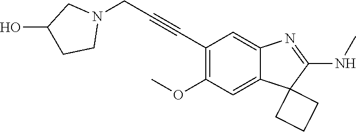 [SMILES: CNC1=Nc2cc(C#CCN3CCC(O)C3)c(OC)cc2C12CCC2] |
| A96 | 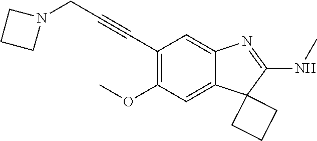 [SMILES: CNC1=Nc2cc(C#CCN3CCC3)c(OC)cc2C12CCC2] |
| A97 | 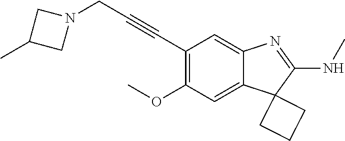 |
| A98 | 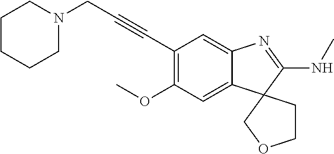 [SMILES: CNC1=Nc2cc(C#CCN3CCCCC3)c(OC)cc2C12CCOC2] |
| A99 | 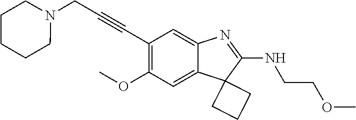 [SMILES: COCCNC1=Nc2cc(C#CCN3CCCCC3)c(OC)cc2C12CCC2] |
| A100 | 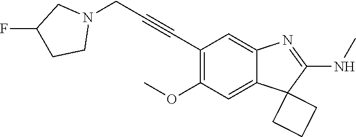 [SMILES: CNC1=Nc2cc(C#CCN3CCC(F)C3)c(OC)cc2C12CCC2] |
| A101 | 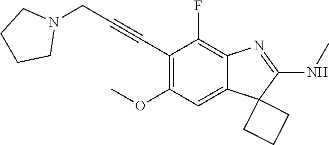 [SMILES: CNC1=Nc2c(cc(OC)c(C#CCN3CCCC3)c2F)C12CCC2] |
| A106 | 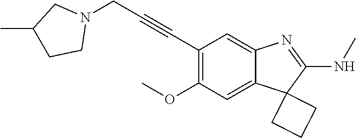 |
| A107 | 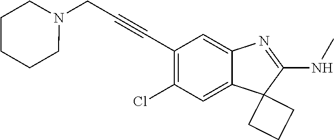 [SMILES: CNC1=Nc2cc(C#CCN3CCCCC3)c(Cl)cc2C12CCC2] |
| A110 | 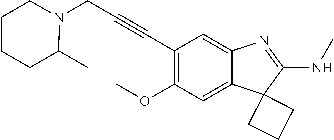 [SMILES: CNC1=Nc2cc(C#CCN3CCCCC3C)c(OC)cc2C12CCC2] |
| A111 | 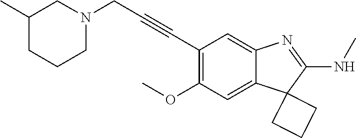 |
| A112 | 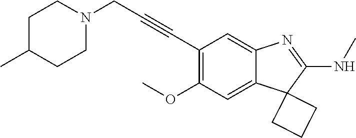 |
| A113 | 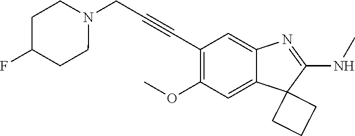 [SMILES: CNC1=Nc2cc(C#CCN3CCC(F)CC3)c(OC)cc2C12CCC2] |
| A114 | 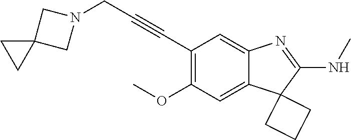 [SMILES: CNC1=Nc2cc(C#CCN3CC4(CC4)C3)c(OC)cc2C12CCC2] |
| A115 | 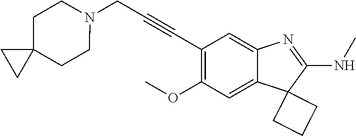 [SMILES: CNC1=Nc2cc(C#CCN3CCC4(CC3)CC4)c(OC)cc2C12CCC2] |
| A116 | 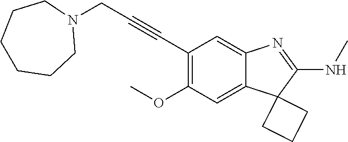 [SMILES: CNC1=Nc2cc(C#CCN3CCCCCC3)c(OC)cc2C12CCC2] |
| A117 | 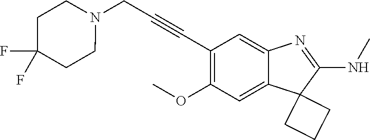 [SMILES: CNC1=Nc2cc(C#CCN3CCC(F)(F)CC3)c(OC)cc2C12CCC2] |
| A118 | 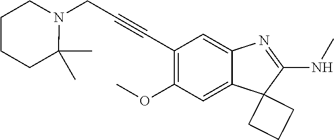 [SMILES: CNC1=Nc2cc(C#CCN3CCCCC3(C)C)c(OC)cc2C12CCC2] |
| A119 | 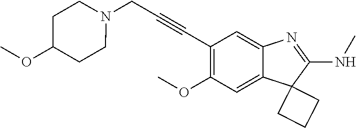 |
| A120 | 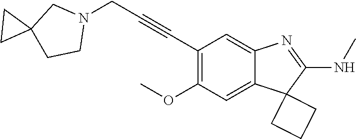 [SMILES: CNC1=Nc2cc(C#CCN3CCC4(CC4)C3)c(OC)cc2C12CCC2] |
| A121 | 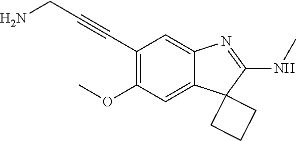 [SMILES: CNC1=Nc2cc(C#CCN)c(OC)cc2C12CCC2] |
| A122 | 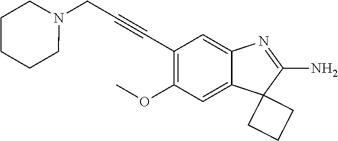 [SMILES: COc1cc2c(cc1C#CCN1CCCCC1)N=C(N)C21CCC1] |
| A123 | 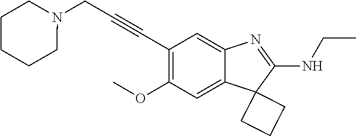 [SMILES: CCNC1=Nc2cc(C#CCN3CCCCC3)c(OC)cc2C12CCC2] |
| A124 | 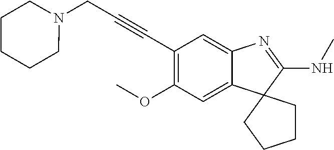 [SMILES: CNC1=Nc2cc(C#CCN3CCCCC3)c(OC)cc2C12CCCC2] |
| A125 | 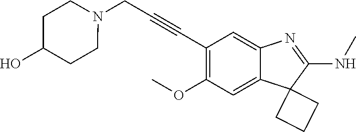 [SMILES: CNC1=Nc2cc(C#CCN3CCC(O)CC3)c(OC)cc2C12CCC2] |
| A126 | 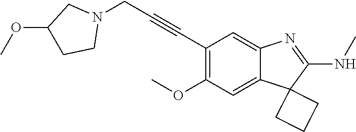 |
| A127 | 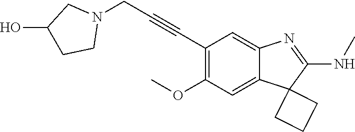 [SMILES: CNC1=Nc2cc(C#CCN3CCC(O)C3)c(OC)cc2C12CCC2] |
| A128 | 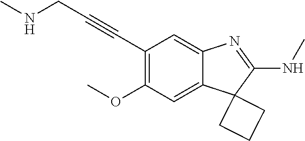 |
| A129 | 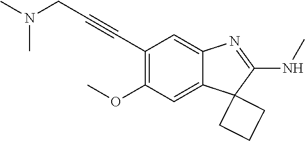 |
| A130 | 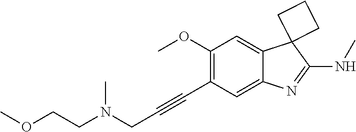 |
| A131 | 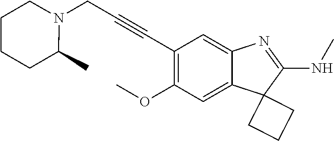 [SMILES: CNC1=Nc2cc(C#CCN3CCCC[C@@H]3C)c(OC)cc2C12CCC2] |
| A132 | 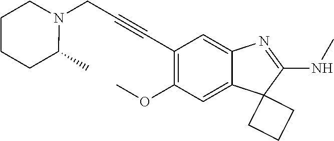 [SMILES: CNC1=Nc2cc(C#CCN3CCCC[C@H]3C)c(OC)cc2C12CCC2] |
| A133 | 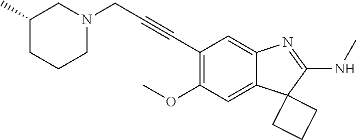 |
| A134 | 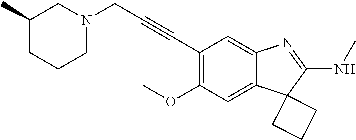 |
| A135 | 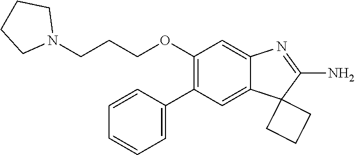 [SMILES: NC1=Nc2cc(OCCCN3CCCC3)c(-c3ccccc3)cc2C12CCC2] |
| A136 | 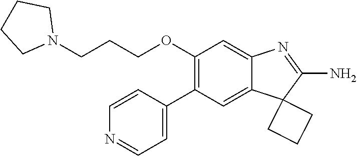 [SMILES: NC1=Nc2cc(OCCCN3CCCC3)c(-c3ccncc3)cc2C12CCC2] |
| A137 | 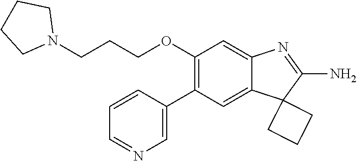 [SMILES: NC1=Nc2cc(OCCCN3CCCC3)c(-c3cccnc3)cc2C12CCC2] |
| A138 | 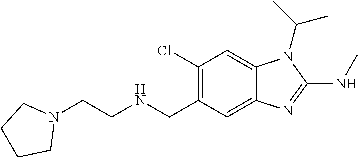 [SMILES: CNc1nc2cc(CNCCN3CCCC3)c(Cl)cc2n1C(C)C] |
| A139 | 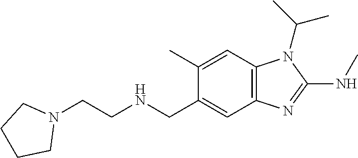 [SMILES: CNc1nc2cc(CNCCN3CCCC3)c(C)cc2n1C(C)C] |
| A140 | 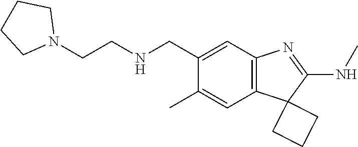 [SMILES: CNC1=Nc2cc(CNCCN3CCCC3)c(C)cc2C12CCC2] |
| A141 | 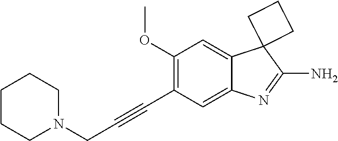 [SMILES: COc1cc2c(cc1C#CCN1CCCCC1)N=C(N)C21CCC1] |

The compounds of Table 2 are the compounds found in U.S. Application Nos. 62/402,863 and 62/509,620, and PCT Appl'n No. PCT/US2017/054468, the entire contents of which are incorporated herein by reference.

TABLE 3

| Cmpd. | |
|---|---|
| No. | Structure |
| B1 | 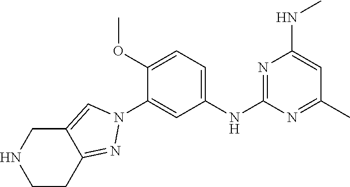 [SMILES: CNc1cc(C)nc(Nc2ccc(OC)c(-n3cc4c(n3)CCNC4)c2)n1] |
| B2 | 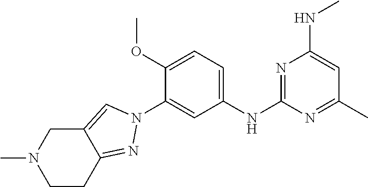 |
| B3 | 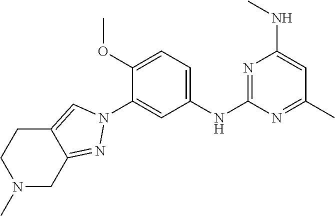 [SMILES: CNc1cc(C)nc(Nc2ccc(OC)c(-n3cc4c(n3)CN(C)CC4)c2)n1] |
| B4 | 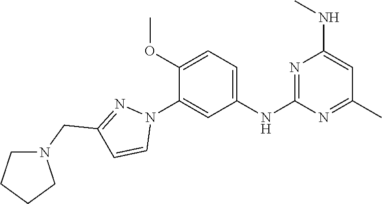 [SMILES: CNc1cc(C)nc(Nc2ccc(OC)c(-n3ccc(CN4CCCC4)n3)c2)n1] |
| B5 | 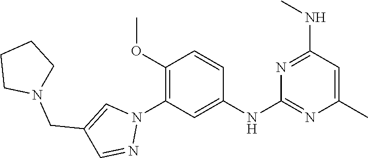 [SMILES: CNc1cc(C)nc(Nc2ccc(OC)c(-n3cc(CN4CCCC4)cn3)c2)n1] |
| B6 | 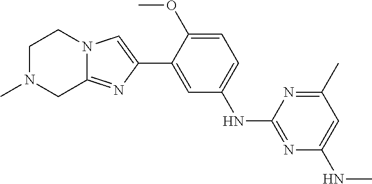 |
| B7 | 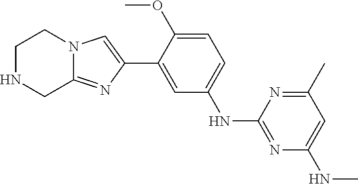 [SMILES: CNc1cc(C)nc(Nc2ccc(OC)c(-c3cn4c(n3)CNCC4)c2)n1] |
| B8 | 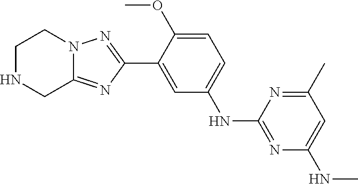 [SMILES: CNc1cc(C)nc(Nc2ccc(OC)c(-c3nc4n(n3)CCNC4)c2)n1] |
| B9 | 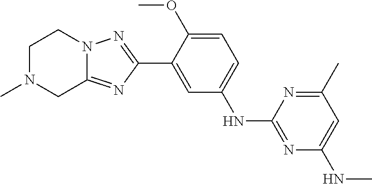 |
| B10 | 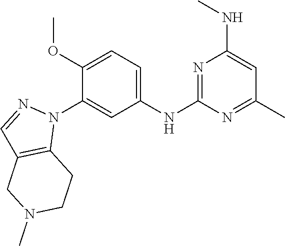 [SMILES: CNc1cc(C)nc(Nc2ccc(OC)c(-n3ncc4c3CCN(C)C4)c2)n1] |
| B11 | 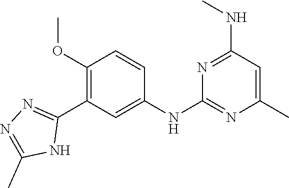 [SMILES: CNc1cc(C)nc(Nc2ccc(OC)c(-c3nnc(C)[nH]3)c2)n1] |
| B12 | 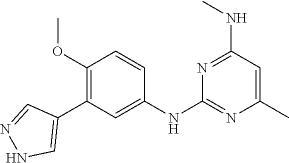 [SMILES: CNc1cc(C)nc(Nc2ccc(OC)c(-c3cn[nH]c3)c2)n1] |
| B13 | 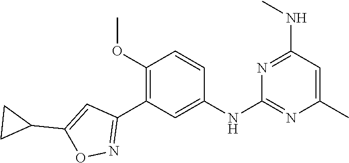 [SMILES: CNc1cc(C)nc(Nc2ccc(OC)c(-c3cc(C4CC4)on3)c2)n1] |
| B14 | 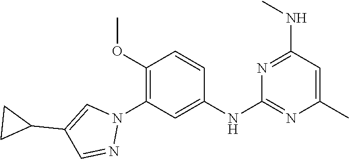 [SMILES: CNc1cc(C)nc(Nc2ccc(OC)c(-n3cc(C4CC4)cn3)c2)n1] |
| B15 | 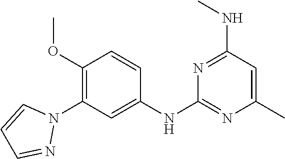 [SMILES: CNc1cc(C)nc(Nc2ccc(OC)c(-n3cccn3)c2)n1] |
| B16 | 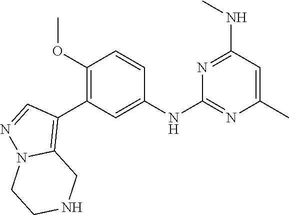 [SMILES: CNc1cc(C)nc(Nc2ccc(OC)c(-c3cnn4c3CNCC4)c2)n1] |
| B17 | 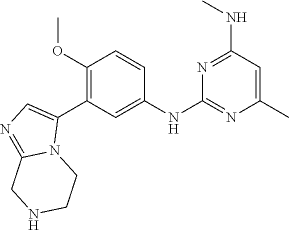 [SMILES: CNc1cc(C)nc(Nc2ccc(OC)c(-c3cnc4n3CCNC4)c2)n1] |
| B18 | 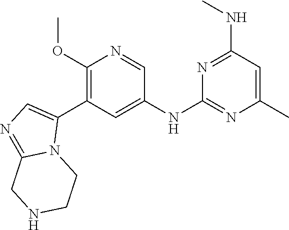 [SMILES: CNc1cc(C)nc(Nc2cnc(OC)c(-c3cnc4n3CCNC4)c2)n1] |
| B19 | 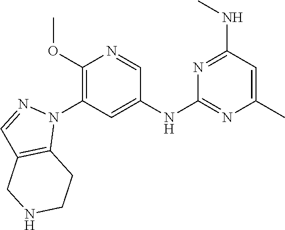 [SMILES: CNc1cc(C)nc(Nc2cnc(OC)c(-n3ncc4c3CCNC4)c2)n1] |
| B20 | 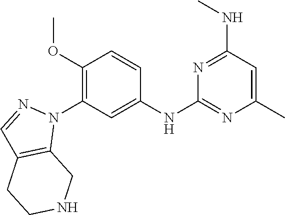 [SMILES: CNc1cc(C)nc(Nc2ccc(OC)c(-n3ncc4c3CNCC4)c2)n1] |
| B21 | 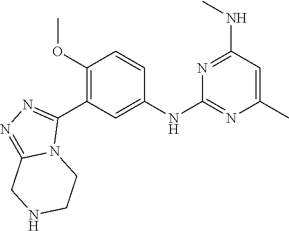 [SMILES: CNc1cc(C)nc(Nc2ccc(OC)c(-c3nnc4n3CCNC4)c2)n1] |
| B22 | 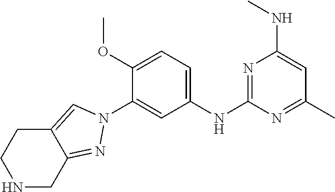 [SMILES: CNc1cc(C)nc(Nc2ccc(OC)c(-n3cc4c(n3)CNCC4)c2)n1] |
| B23 | 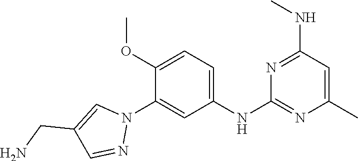 [SMILES: CNc1cc(C)nc(Nc2ccc(OC)c(-n3cc(CN)cn3)c2)n1] |
| B24 | 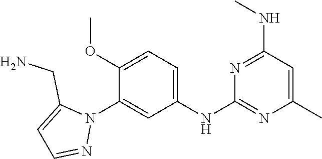 [SMILES: CNc1cc(C)nc(Nc2ccc(OC)c(-n3nccc3CN)c2)n1] |
| B25 | 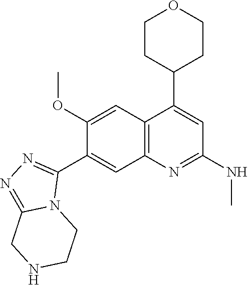 [SMILES: CNc1cc(C2CCOCC2)c2cc(OC)c(-c3nnc4n3CCNC4)cc2n1] |
| B26 | 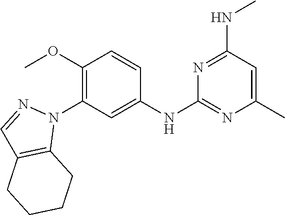 [SMILES: CNc1cc(C)nc(Nc2ccc(OC)c(-n3ncc4c3CCCC4)c2)n1] |
| B27 | 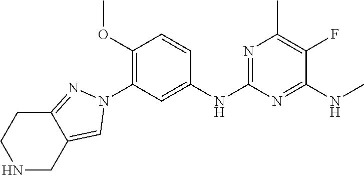 [SMILES: CNc1nc(Nc2ccc(OC)c(-n3cc4c(n3)CCNC4)c2)nc(C)c1F] |
| B28 | 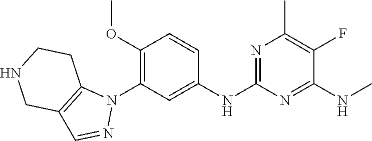 [SMILES: CNc1nc(Nc2ccc(OC)c(-n3ncc4c3CCNC4)c2)nc(C)c1F] |
| B29 | 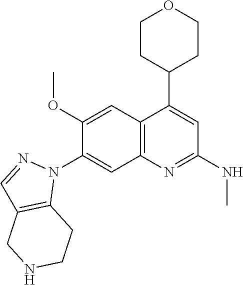 [SMILES: CNc1cc(C2CCOCC2)c2cc(OC)c(-n3ncc4c3CCNC4)cc2n1] |
| B30 | 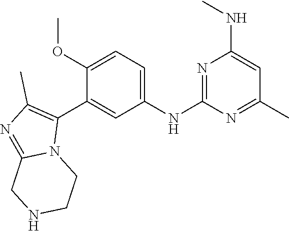 [SMILES: CNc1cc(C)nc(Nc2ccc(OC)c(-c3c(C)nc4n3CCNC4)c2)n1] |
| B31 | 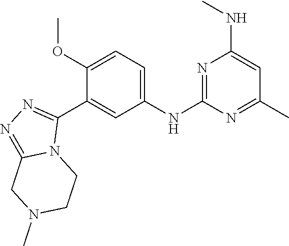 [SMILES: CNc1cc(C)nc(Nc2ccc(OC)c(-c3nnc4n3CCN(C)C4)c2)n1] |
| B32 | 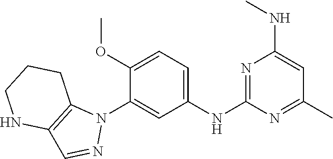 [SMILES: CNc1cc(C)nc(Nc2ccc(OC)c(-n3ncc4c3CCCN4)c2)n1] |
| B33 | 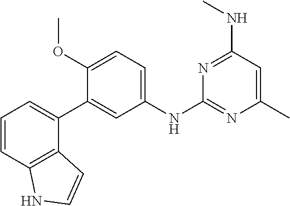 [SMILES: CNc1cc(C)nc(Nc2ccc(OC)c(-c3cccc4[nH]ccc34)c2)n1] |
| B34 | 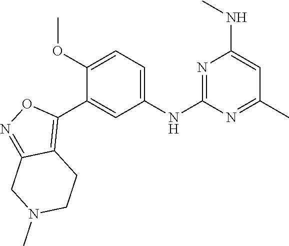 [SMILES: CNc1cc(C)nc(Nc2ccc(OC)c(-c3onc4c3CCN(C)C4)c2)n1] |
| B35 | 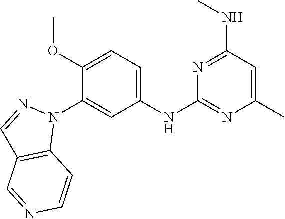 [SMILES: CNc1cc(C)nc(Nc2ccc(OC)c(-n3ncc4cnccc43)c2)n1] |
| B36 | 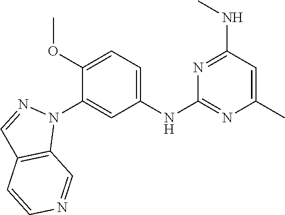 [SMILES: CNc1cc(C)nc(Nc2ccc(OC)c(-n3ncc4ccncc43)c2)n1] |
| B37 | 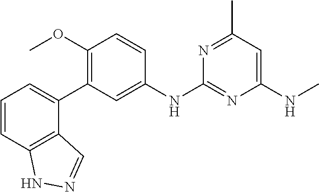 [SMILES: CNc1cc(C)nc(Nc2ccc(OC)c(-c3cccc4[nH]ncc34)c2)n1] |
| B38 | 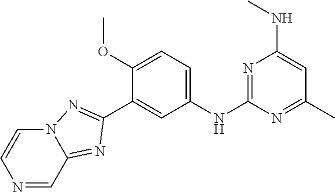 [SMILES: CNc1cc(C)nc(Nc2ccc(OC)c(-c3nc4cnccn4n3)c2)n1] |
| B39 | 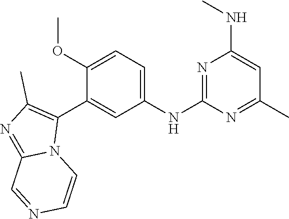 [SMILES: CNc1cc(C)nc(Nc2ccc(OC)c(-c3c(C)nc4cnccn34)c2)n1] |
| B40 | 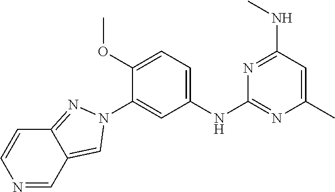 [SMILES: CNc1cc(C)nc(Nc2ccc(OC)c(-n3cc4cnccc4n3)c2)n1] |
| B41 | 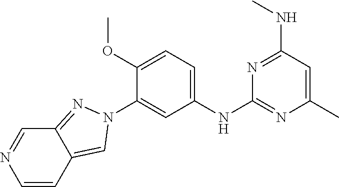 [SMILES: CNc1cc(C)nc(Nc2ccc(OC)c(-n3cc4ccncc4n3)c2)n1] |
| B42 | 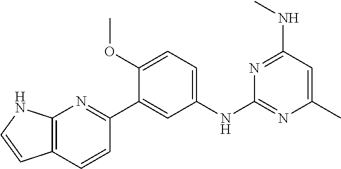 [SMILES: CNc1cc(C)nc(Nc2ccc(OC)c(-c3ccc4cc[nH]c4n3)c2)n1] |
| B43 | 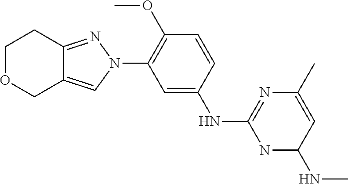 [SMILES: CNC1C=C(C)N=C(Nc2ccc(OC)c(-n3cc4c(n3)CCOC4)c2)N1] |
| B44 | 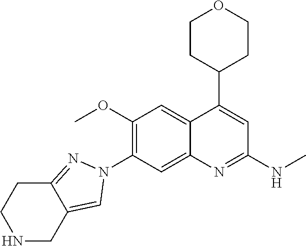 [SMILES: CNc1cc(C2CCOCC2)c2cc(OC)c(-n3cc4c(n3)CCNC4)cc2n1] |
| B45 | 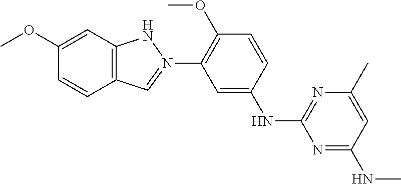 |
| B46 | 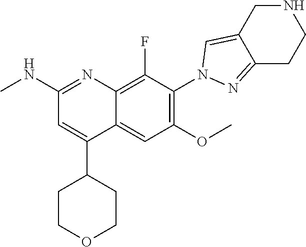 [SMILES: CNc1cc(C2CCOCC2)c2cc(OC)c(-n3cc4c(n3)CCNC4)c(F)c2n1] |
| B47 | 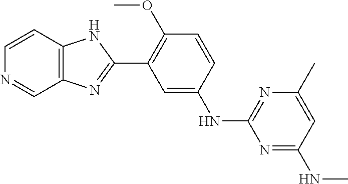 [SMILES: CNc1cc(C)nc(Nc2ccc(OC)c(-c3nc4cnccc4[nH]3)c2)n1] |
| B48 | 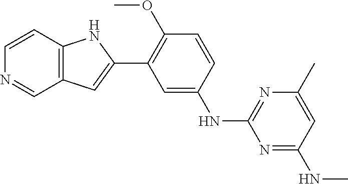 [SMILES: CNc1cc(C)nc(Nc2ccc(OC)c(-c3cc4cnccc4[nH]3)c2)n1] |
| B49 | 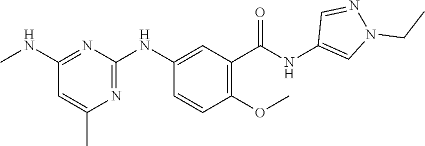 [SMILES: CCn1cc(NC(=O)c2cc(Nc3nc(C)cc(NC)n3)ccc2OC)cn1] |
| B50 | 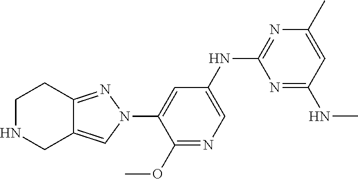 [SMILES: CNc1cc(C)nc(Nc2cnc(OC)c(-n3cc4c(n3)CCNC4)c2)n1] |
| B51 | 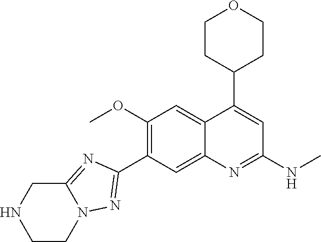 [SMILES: CNc1cc(C2CCOCC2)c2cc(OC)c(-c3nc4n(n3)CCNC4)cc2n1] |
| B52 | 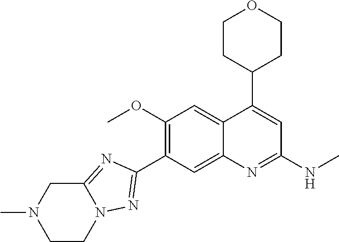 |
| B53 | 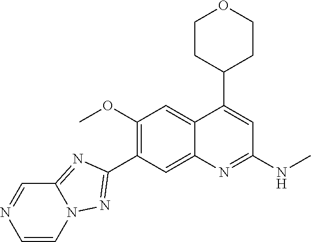 [SMILES: CNc1cc(C2CCOCC2)c2cc(OC)c(-c3nc4cnccn4n3)cc2n1] |
| B54 | 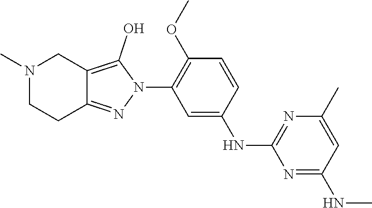 |
| B55 | 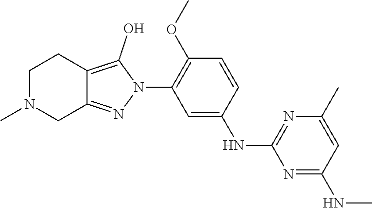 |
| B56 | 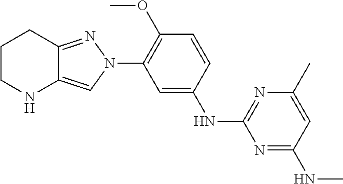 [SMILES: CNc1cc(C)nc(Nc2ccc(OC)c(-n3cc4c(n3)CCCN4)c2)n1] |
| B57 | 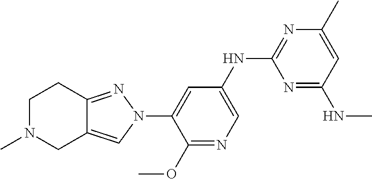 |
| B58 | 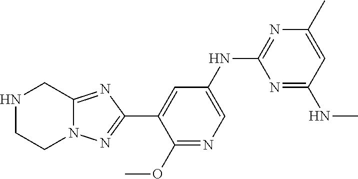 [SMILES: CNc1cc(C)nc(Nc2cnc(OC)c(-c3nc4n(n3)CCNC4)c2)n1] |
| B59 | 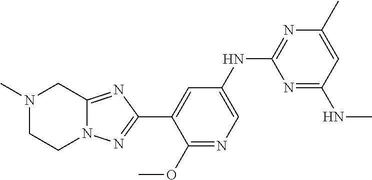 |
| B60 | 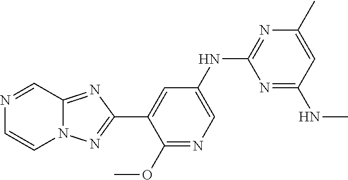 [SMILES: CNc1cc(C)nc(Nc2cnc(OC)c(-c3nc4cnccn4n3)c2)n1] |
| B61 | 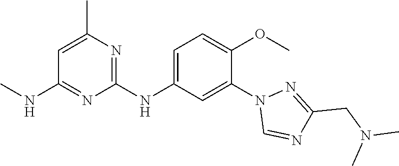 |
| B62 | 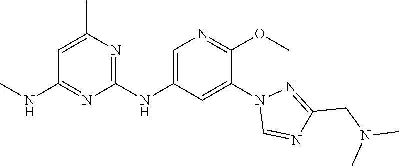 |
| B63 | 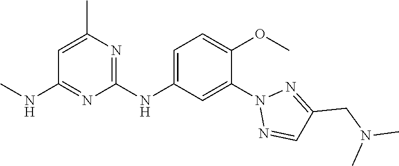 |
| B64 | 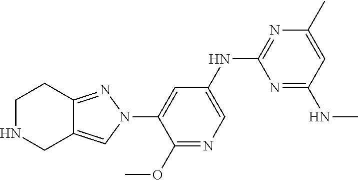 [SMILES: CNc1cc(C)nc(Nc2cnc(OC)c(-n3cc4c(n3)CCNC4)c2)n1] |
| B65 | 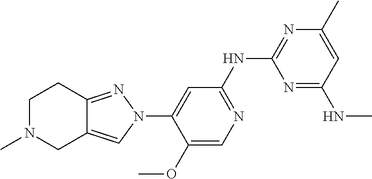 |
| B66 | 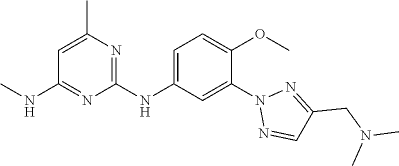 |
| B67 | 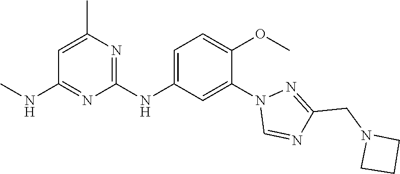 [SMILES: CNc1cc(C)nc(Nc2ccc(OC)c(-n3cnc(CN4CCC4)n3)c2)n1] |
| B68 | 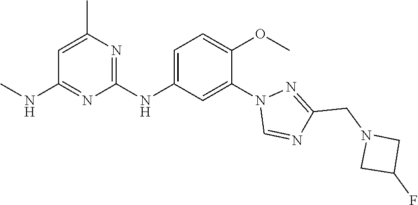 [SMILES: CNc1cc(C)nc(Nc2ccc(OC)c(-n3cnc(CN4CC(F)C4)n3)c2)n1] |
| B69 | 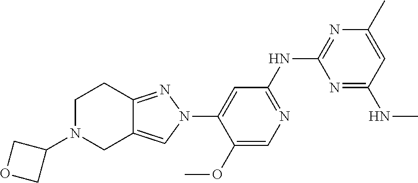 [SMILES: CNc1cc(C)nc(Nc2cc(-n3cc4c(n3)CCN(C3COC3)C4)c(OC)cn2)n1] |
| B70 | 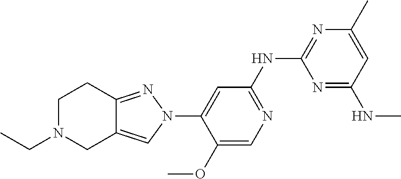 [SMILES: CCN1CCc2nn(-c3cc(Nc4nc(C)cc(NC)n4)ncc3OC)cc2C1] |
| B71 | 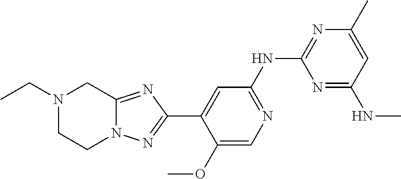 [SMILES: CCN1CCn2nc(-c3cc(Nc4nc(C)cc(NC)n4)ncc3OC)nc2C1] |
| B72 | 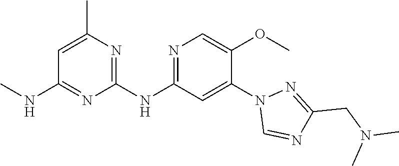 |
| B73 | 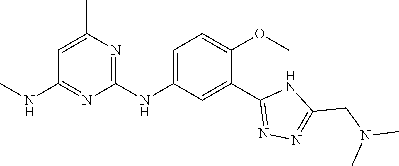 |
| B74 | 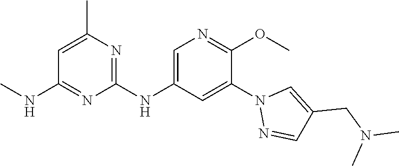 |
| B75 | 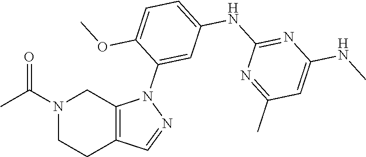 [SMILES: CNc1cc(C)nc(Nc2ccc(OC)c(-n3ncc4c3CN(C(C)=O)CC4)c2)n1] |
| B76 | 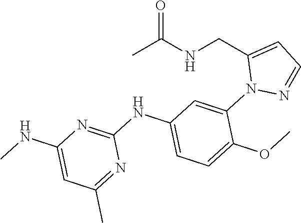 [SMILES: CNc1cc(C)nc(Nc2ccc(OC)c(-n3nccc3CNC(C)=O)c2)n1] |
| B77 | 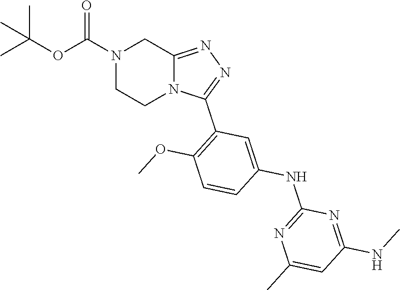 [SMILES: CNc1cc(C)nc(Nc2ccc(OC)c(-c3nnc4n3CCN(C(=O)OC(C)(C)C)C4)c2)n1] |
| B78 | 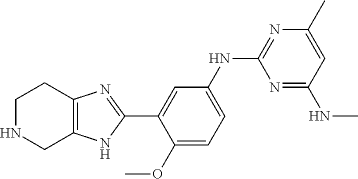 [SMILES: CNc1cc(C)nc(Nc2ccc(OC)c(-c3nc4c([nH]3)CNCC4)c2)n1] |
| B79 | 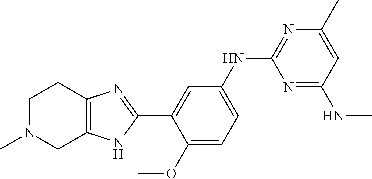 |
| B80 | 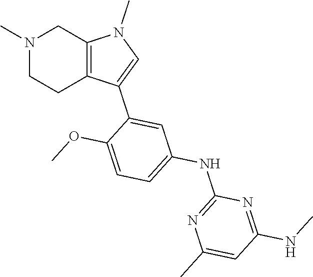 |
| B81 | 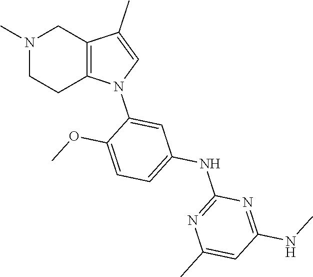 |
| B82 | 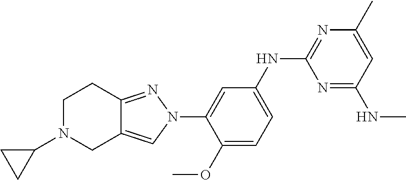 [SMILES: CNc1cc(C)nc(Nc2ccc(OC)c(-n3cc4c(n3)CCN(C3CC3)C4)c2)n1] |
| B83 | 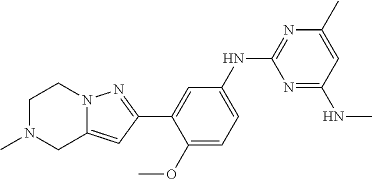 |
| B84 | 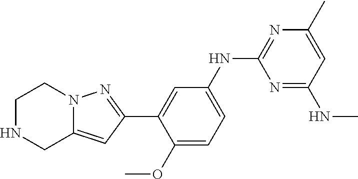 [SMILES: CNc1cc(C)nc(Nc2ccc(OC)c(-c3cc4n(n3)CCNC4)c2)n1] |
| B85 | 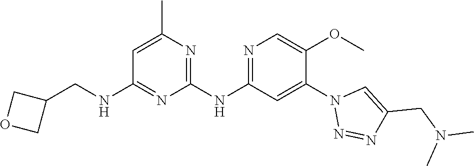 [SMILES: COc1cnc(Nc2nc(C)cc(NCC3COC3)n2)cc1-n1cc(CN(C)C)nn1] |
| B86 | 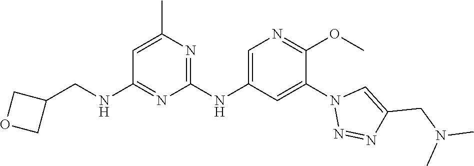 [SMILES: COc1ncc(Nc2nc(C)cc(NCC3COC3)n2)cc1-n1cc(CN(C)C)nn1] |
| B87 | 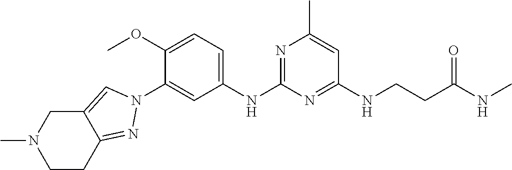 |
| B88 | 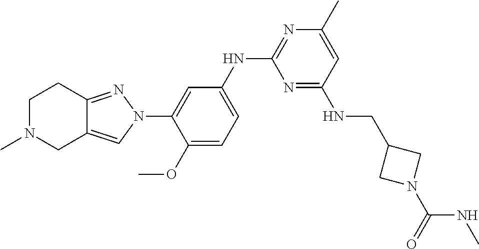 |
| B89 | 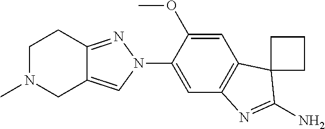 [SMILES: COc1cc2c(cc1-n1cc3c(n1)CCN(C)C3)N=C(N)C21CCC1] |
| B90 | 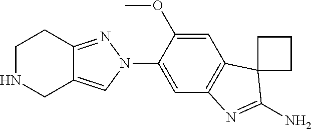 [SMILES: COc1cc2c(cc1-n1cc3c(n1)CCNC3)N=C(N)C21CCC1] |
| B91 | 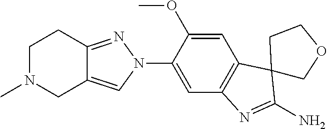 [SMILES: COc1cc2c(cc1-n1cc3c(n1)CCN(C)C3)N=C(N)C21CCOC1] |
| B92 | 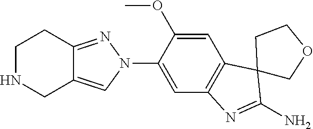 [SMILES: COc1cc2c(cc1-n1cc3c(n1)CCNC3)N=C(N)C21CCOC1] |
| B93 | 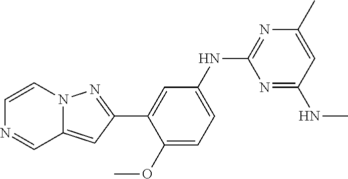 [SMILES: CNc1cc(C)nc(Nc2ccc(OC)c(-c3cc4cnccn4n3)c2)n1] |
| B94 | 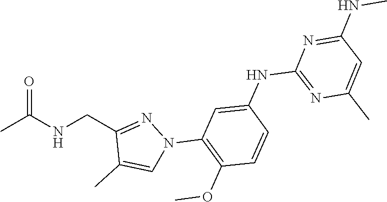 [SMILES: CNc1cc(C)nc(Nc2ccc(OC)c(-n3cc(C)c(CNC(C)=O)n3)c2)n1] |
| B95 | 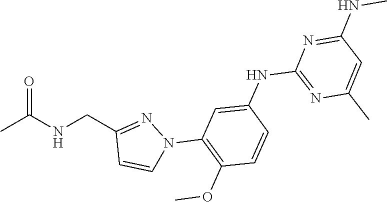 [SMILES: CNc1cc(C)nc(Nc2ccc(OC)c(-n3ccc(CNC(C)=O)n3)c2)n1] |
| B96 | 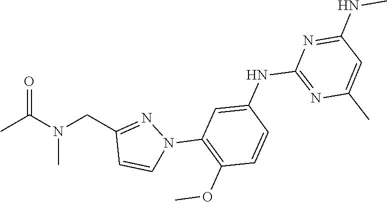 [SMILES: CNc1cc(C)nc(Nc2ccc(OC)c(-n3ccc(CN(C)C(C)=O)n3)c2)n1] |
| B97 | 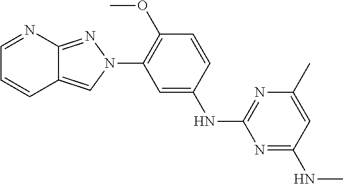 [SMILES: CNc1cc(C)nc(Nc2ccc(OC)c(-n3cc4cccnc4n3)c2)n1] |
| B98 | 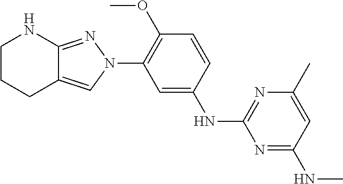 [SMILES: CNc1cc(C)nc(Nc2ccc(OC)c(-n3cc4c(n3)NCCC4)c2)n1] |
| B99 | 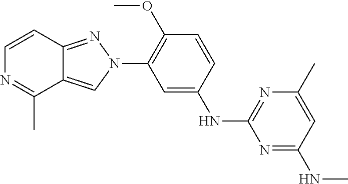 [SMILES: CNc1cc(C)nc(Nc2ccc(OC)c(-n3cc4c(C)nccc4n3)c2)n1] |
| B100 | 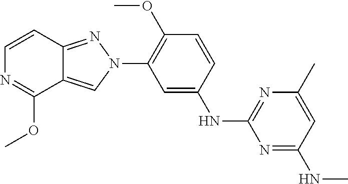 [SMILES: CNc1cc(C)nc(Nc2ccc(OC)c(-n3cc4c(OC)nccc4n3)c2)n1] |
| B101 | 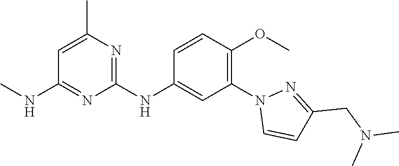 |
| B102 | 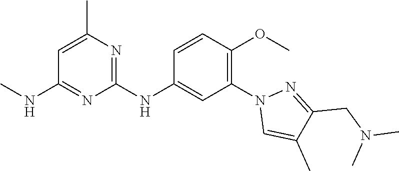 |
| B103 | 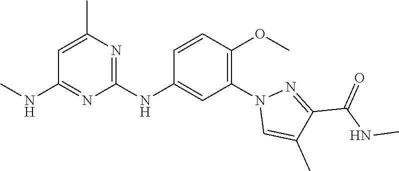 |
| B104 | 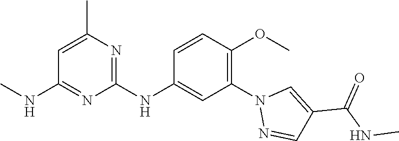 |
| B105 | 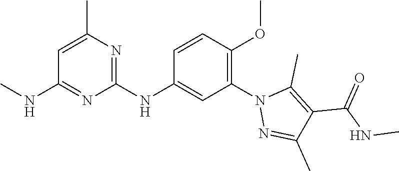 |
| B106 | 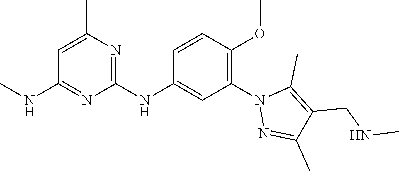 |
| B107 | 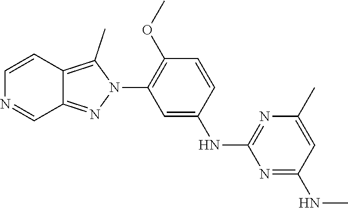 [SMILES: CNc1cc(C)nc(Nc2ccc(OC)c(-n3nc4cnccc4c3C)c2)n1] |
| B108 | 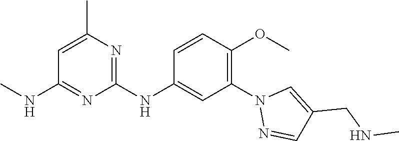 |
| B109 | 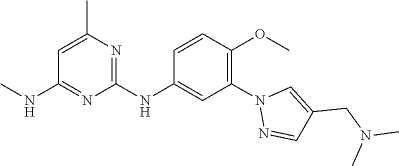 |
| B110 | 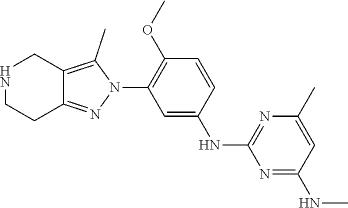 [SMILES: CNc1cc(C)nc(Nc2ccc(OC)c(-n3nc4c(c3C)CNCC4)c2)n1] |
| B111 | 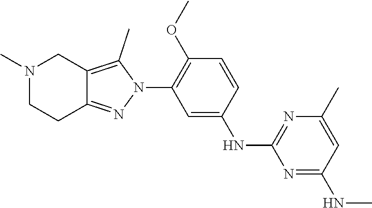 |
| B112 | 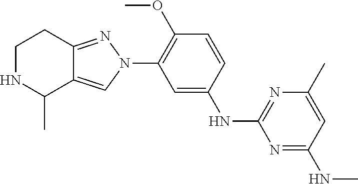 [SMILES: CNc1cc(C)nc(Nc2ccc(OC)c(-n3cc4c(n3)CCNC4C)c2)n1] |
| B113 | 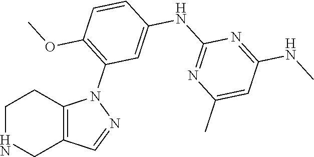 [SMILES: CNc1cc(C)nc(Nc2ccc(OC)c(-n3ncc4c3CCNC4)c2)n1] |
| B114 | 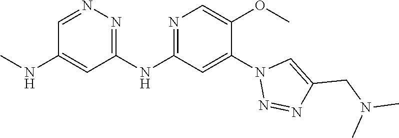 |
| B115 | 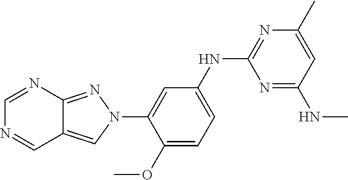 [SMILES: CNc1cc(C)nc(Nc2ccc(OC)c(-n3cc4cncnc4n3)c2)n1] |
| B116 | 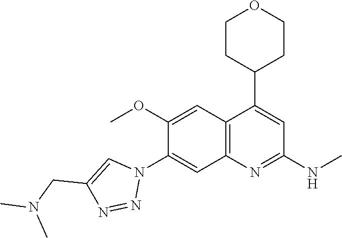 |
| B117 | 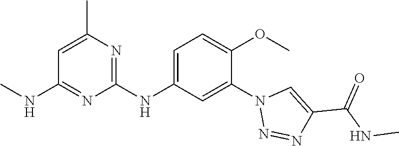 |
| B118 | 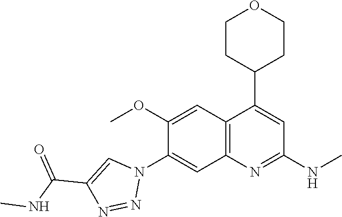 |
| B119 | 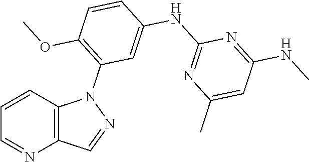 [SMILES: CNc1cc(C)nc(Nc2ccc(OC)c(-n3ncc4ncccc43)c2)n1] |
| B120 | 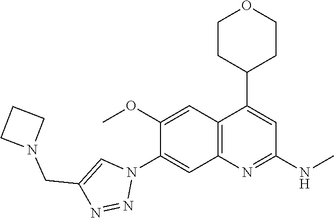 [SMILES: CNc1cc(C2CCOCC2)c2cc(OC)c(-n3cc(CN4CCC4)nn3)cc2n1] |
| B121 | 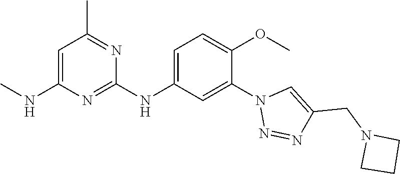 [SMILES: CNc1cc(C)nc(Nc2ccc(OC)c(-n3cc(CN4CCC4)nn3)c2)n1] |
| B122 | 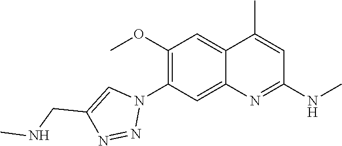 |
| B123 | 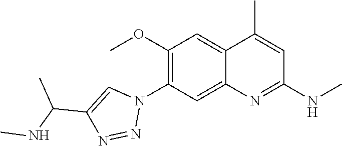 |
| B124 | 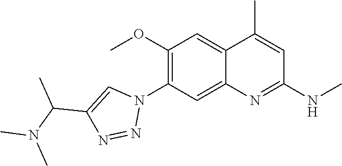 |
| B125 | 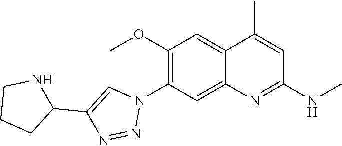 [SMILES: CNc1cc(C)c2cc(OC)c(-n3cc(C4CCCN4)nn3)cc2n1] |
| B126 | 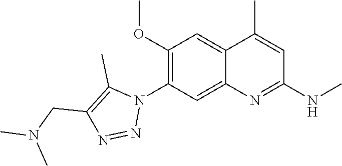 |
| B127 | 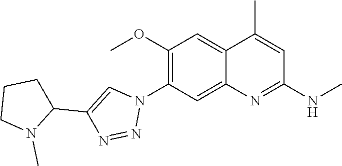 [SMILES: CNc1cc(C)c2cc(OC)c(-n3cc(C4CCCN4C)nn3)cc2n1] |
| B128 | 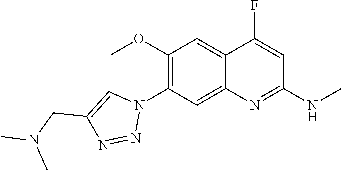 |
| B129 | 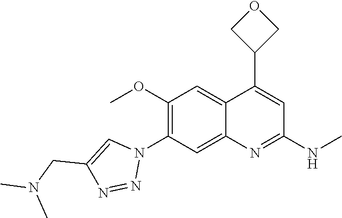 |
| B130 | 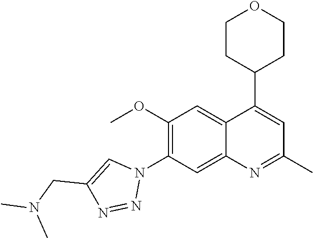 |
| B131 | 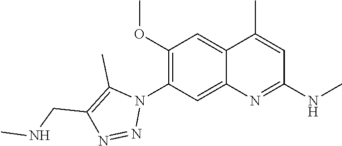 |
| B132 | 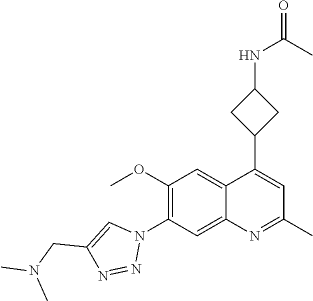 [SMILES: COc1cc2c(C3CC(NC(C)=O)C3)cc(C)nc2cc1-n1cc(CN(C)C)nn1] |
| B133 | 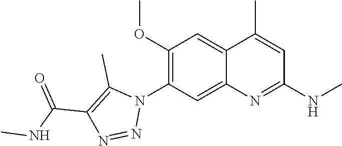 |
| B134 | 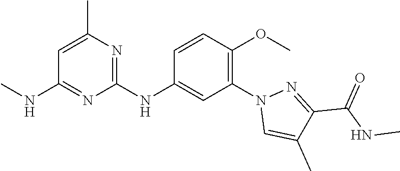 |
| B135 | 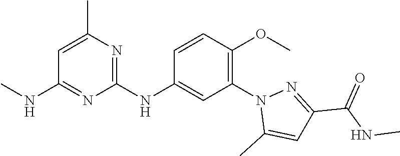 |
| B136 | 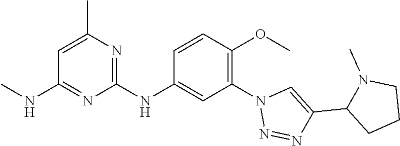 [SMILES: CNc1cc(C)nc(Nc2ccc(OC)c(-n3cc(C4CCCN4C)nn3)c2)n1] |
| B137 | 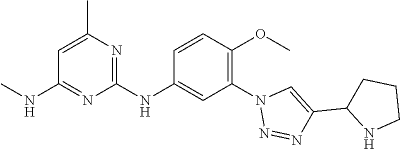 [SMILES: CNc1cc(C)nc(Nc2ccc(OC)c(-n3cc(C4CCCN4)nn3)c2)n1] |
| B138 | 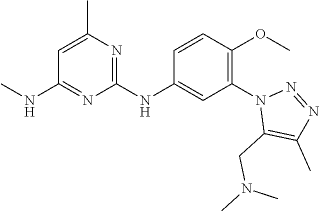 [SMILES: CNc1cc(C)nc(Nc2ccc(OC)c(-n3nnc(C)c3CN(C)C)c2)n1] |
| B139 | 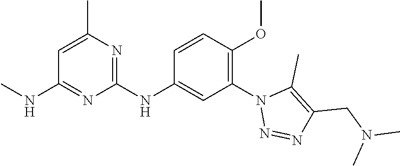 |
| B140 | 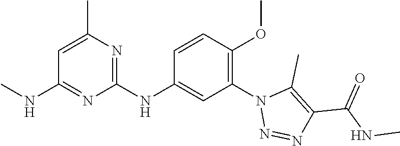 |
| B141 | 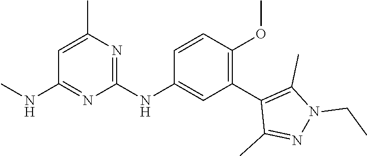 [SMILES: CCn1nc(C)c(-c2cc(Nc3nc(C)cc(NC)n3)ccc2OC)c1C] |
| B142 | 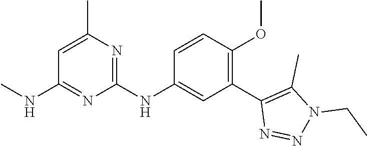 [SMILES: CCn1nnc(-c2cc(Nc3nc(C)cc(NC)n3)ccc2OC)c1C] |
| B143 | 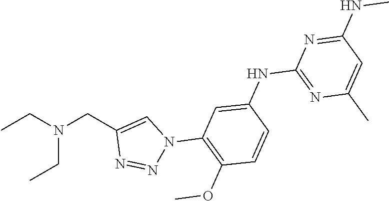 [SMILES: CCN(CC)Cc1cn(-c2cc(Nc3nc(C)cc(NC)n3)ccc2OC)nn1] |
| B144 | 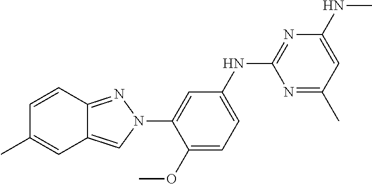 |
| B145 | 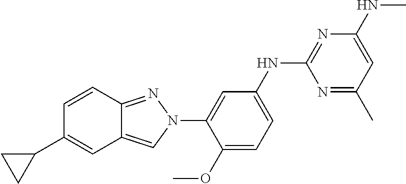 [SMILES: CNc1cc(C)nc(Nc2ccc(OC)c(-n3cc4cc(C5CC5)ccc4n3)c2)n1] |
| B146 | 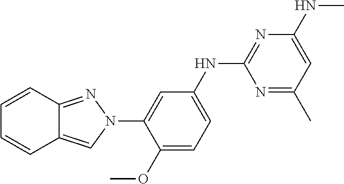 [SMILES: CNc1cc(C)nc(Nc2ccc(OC)c(-n3cc4ccccc4n3)c2)n1] |
| B147 | 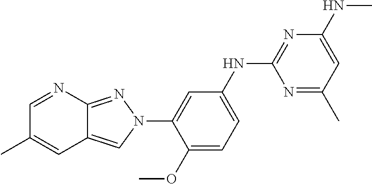 |
| B148 | 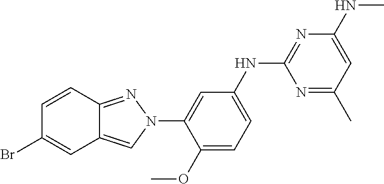 [SMILES: CNc1cc(C)nc(Nc2ccc(OC)c(-n3cc4cc(Br)ccc4n3)c2)n1] |
| B149 | 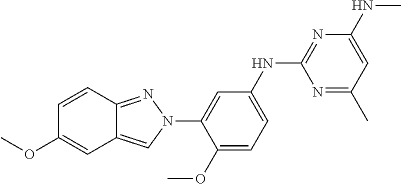 |
| B150 | 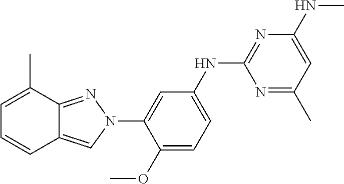 [SMILES: CNc1cc(C)nc(Nc2ccc(OC)c(-n3cc4cccc(C)c4n3)c2)n1] |
| B151 | 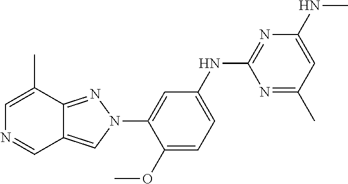 [SMILES: CNc1cc(C)nc(Nc2ccc(OC)c(-n3cc4cncc(C)c4n3)c2)n1] |
| B152 | 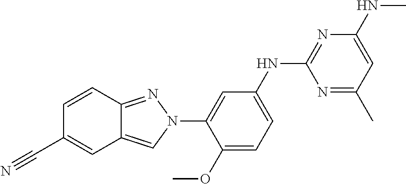 [SMILES: CNc1cc(C)nc(Nc2ccc(OC)c(-n3cc4cc(C#N)ccc4n3)c2)n1] |
| B153 | 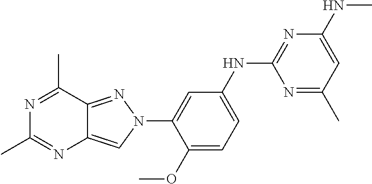 |
| B154 | 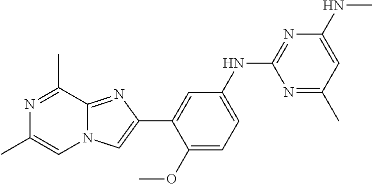 |
| B155 | 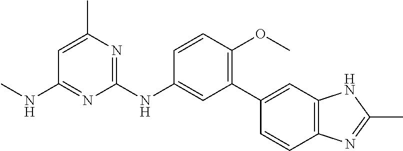 |
| B156 | 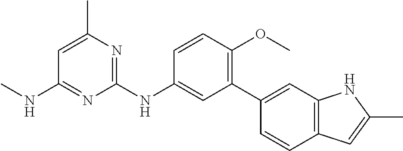 |
| B157 | 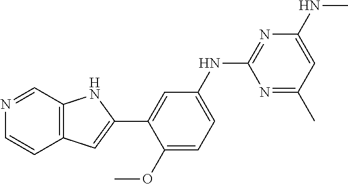 [SMILES: CNc1cc(C)nc(Nc2ccc(OC)c(-c3cc4ccncc4[nH]3)c2)n1] |
| B158 | 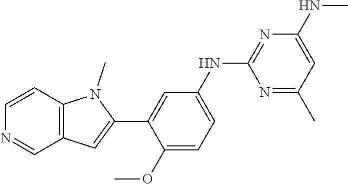 [SMILES: CNc1cc(C)nc(Nc2ccc(OC)c(-c3cc4cnccc4n3C)c2)n1] |
| B159 | 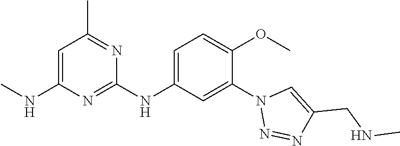 |
| B160 | 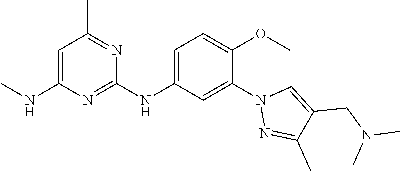 |
| B161 | 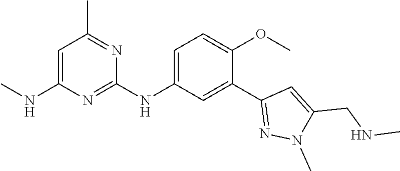 |
| B162 | 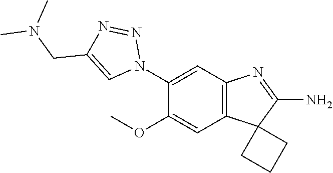 [SMILES: COc1cc2c(cc1-n1cc(CN(C)C)nn1)N=C(N)C21CCC1] |
| B163 | 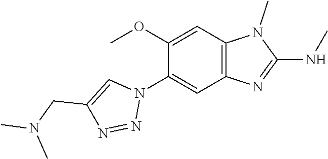 |
| B164 | 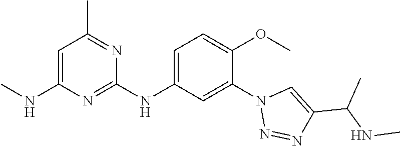 |
| B165 | 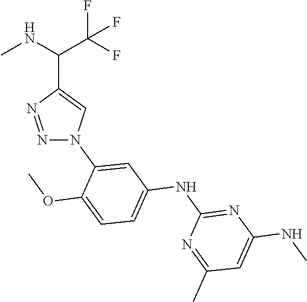 |
| B166 | 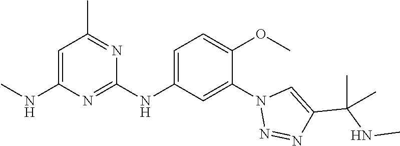 |
| B167 | 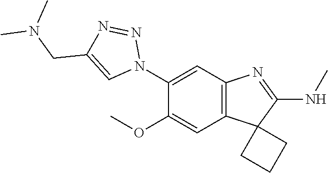 |
| B168 | 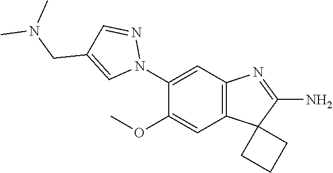 [SMILES: COc1cc2c(cc1-n1cc(CN(C)C)cn1)N=C(N)C21CCC1] |
| B169 | 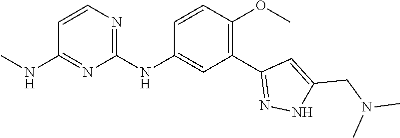 |
| B170 | 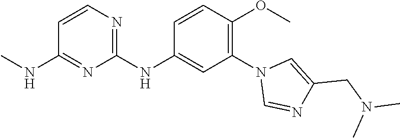 |
| B171 | 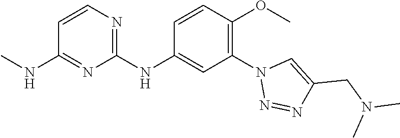 |
| B172 | 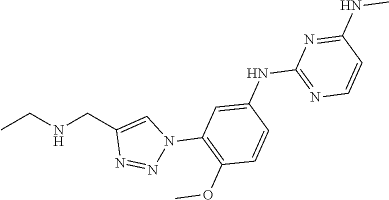 [SMILES: CCNCc1cn(-c2cc(Nc3nccc(NC)n3)ccc2OC)nn1] |
| B173 | 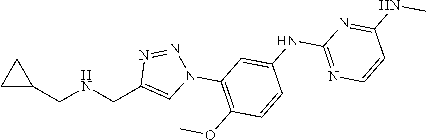 [SMILES: CNc1ccnc(Nc2ccc(OC)c(-n3cc(CNCC4CC4)nn3)c2)n1] |
| B174 | 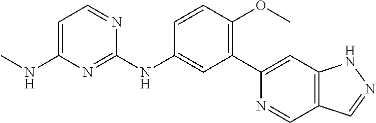 [SMILES: CNc1ccnc(Nc2ccc(OC)c(-c3cc4[nH]ncc4cn3)c2)n1] |
| B175 | 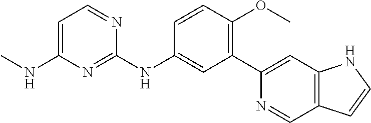 [SMILES: CNc1ccnc(Nc2ccc(OC)c(-c3cc4[nH]ccc4cn3)c2)n1] |
| B176 | 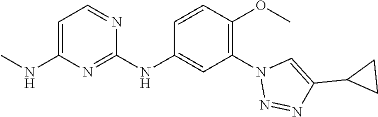 [SMILES: CNc1ccnc(Nc2ccc(OC)c(-n3cc(C4CC4)nn3)c2)n1] |
| B177 | 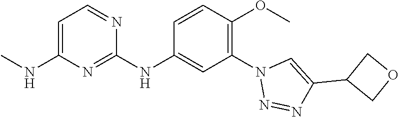 [SMILES: CNc1ccnc(Nc2ccc(OC)c(-n3cc(C4COC4)nn3)c2)n1] |
| B178 | 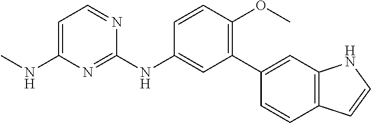 [SMILES: CNc1ccnc(Nc2ccc(OC)c(-c3ccc4cc[nH]c4c3)c2)n1] |
| B179 | 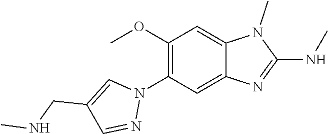 |
| B180 | 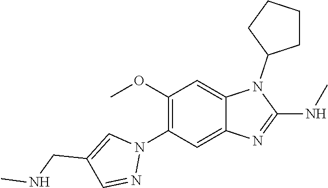 |
| B181 | 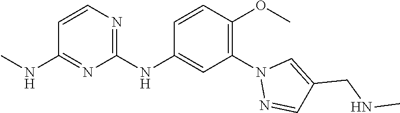 |
| B182 | 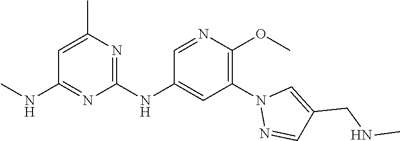 |
| B183 | 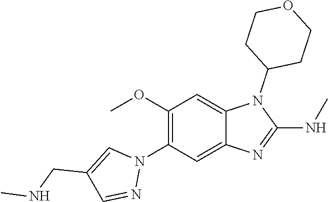 |
| B184 | 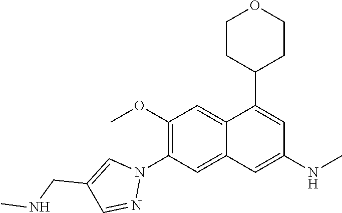 |
| B185 | 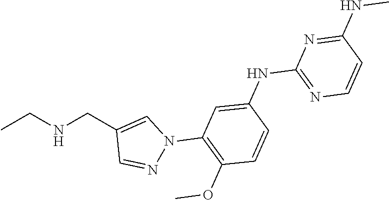 [SMILES: CCNCc1cnn(-c2cc(Nc3nccc(NC)n3)ccc2OC)c1] |
| B186 | 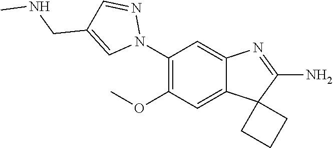 [SMILES: CNCc1cnn(-c2cc3c(cc2OC)C2(CCC2)C(N)=N3)c1] |
| B187 | 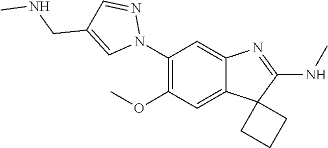 |
| B188 | 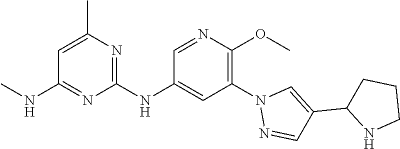 [SMILES: CNc1cc(C)nc(Nc2cnc(OC)c(-n3cc(C4CCCN4)cn3)c2)n1] |
| B191 | 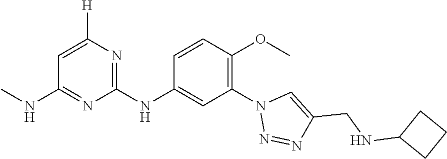 [SMILES: CNc1ccnc(Nc2ccc(OC)c(-n3cc(CNC4CCC4)nn3)c2)n1] |
| B192 | 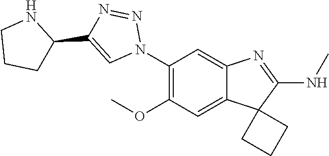 [SMILES: CNC1=Nc2cc(-n3cc([C@H]4CCCN4)nn3)c(OC)cc2C12CCC2] |
| B193 | 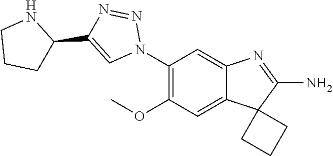 [SMILES: COc1cc2c(cc1-n1cc([C@H]3CCCN3)nn1)N=C(N)C21CCC1] |
| B194 | 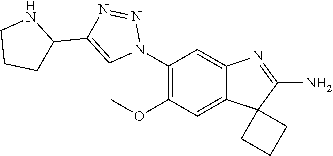 [SMILES: COc1cc2c(cc1-n1cc(C3CCCN3)nn1)N=C(N)C21CCC1] |
| B195 | 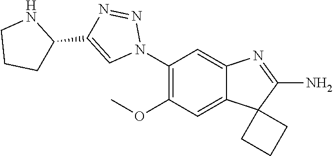 [SMILES: COc1cc2c(cc1-n1cc([C@@H]3CCCN3)nn1)N=C(N)C21CCC1] |
| B196 | 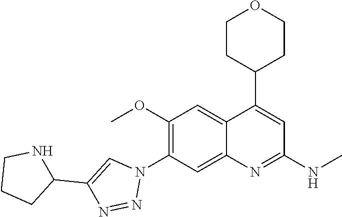 [SMILES: CNc1cc(C2CCOCC2)c2cc(OC)c(-n3cc(C4CCCN4)nn3)cc2n1] |
| B197 | 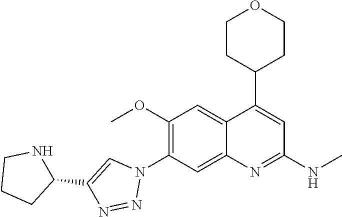 [SMILES: CNc1cc(C2CCOCC2)c2cc(OC)c(-n3cc([C@@H]4CCCN4)nn3)cc2n1] |
| B198 | 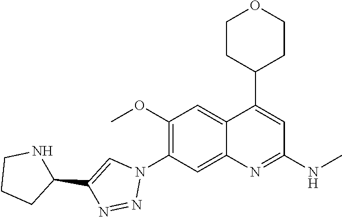 [SMILES: CNc1cc(C2CCOCC2)c2cc(OC)c(-n3cc([C@H]4CCCN4)nn3)cc2n1] |
| B199 | 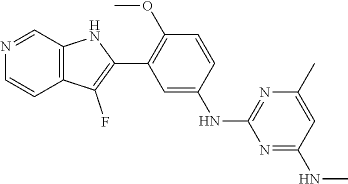 [SMILES: CNc1cc(C)nc(Nc2ccc(OC)c(-c3[nH]c4cnccc4c3F)c2)n1] |
| B200 | 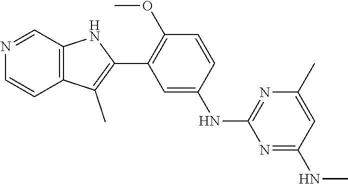 [SMILES: CNc1cc(C)nc(Nc2ccc(OC)c(-c3[nH]c4cnccc4c3C)c2)n1] |
| B201 | 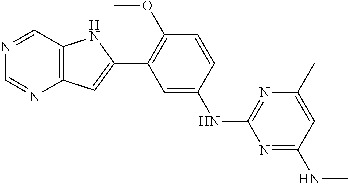 [SMILES: CNc1cc(C)nc(Nc2ccc(OC)c(-c3cc4ncncc4[nH]3)c2)n1] |
| B202 | 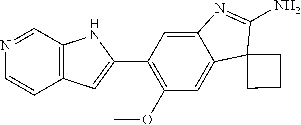 [SMILES: COc1cc2c(cc1-c1cc3ccncc3[nH]1)N=C(N)C21CCC1] |
| B203 | 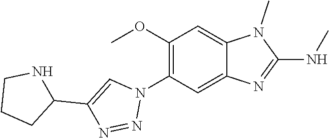 [SMILES: CNc1nc2cc(-n3cc(C4CCCN4)nn3)c(OC)cc2n1C] |
| B204 | 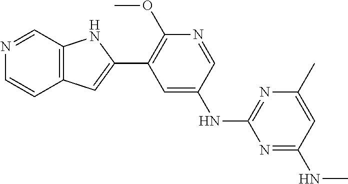 [SMILES: CNc1cc(C)nc(Nc2cnc(OC)c(-c3cc4ccncc4[nH]3)c2)n1] |
| B205 | 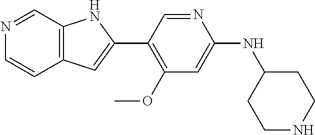 [SMILES: COc1cc(NC2CCNCC2)ncc1-c1cc2ccncc2[nH]1] |
| B206 | 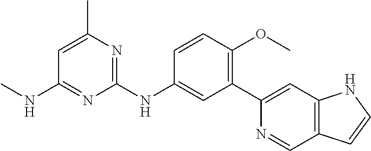 [SMILES: CNc1cc(C)nc(Nc2ccc(OC)c(-c3cc4[nH]ccc4cn3)c2)n1] |
| B207 | 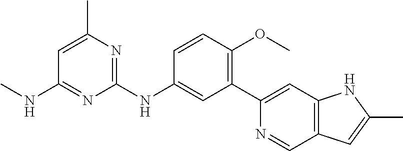 |
| B208 | 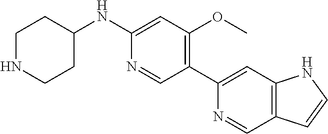 [SMILES: COc1cc(NC2CCNCC2)ncc1-c1cc2[nH]ccc2cn1] |
| B209 | 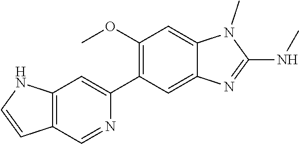 [SMILES: CNc1nc2cc(-c3cc4[nH]ccc4cn3)c(OC)cc2n1C] |
| B210 | 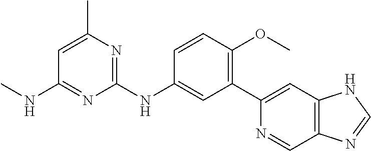 [SMILES: CNc1cc(C)nc(Nc2ccc(OC)c(-c3cc4[nH]cnc4cn3)c2)n1] |
| B211 | 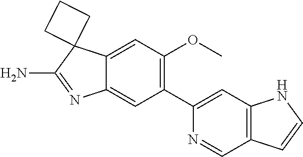 [SMILES: COc1cc2c(cc1-c1cc3[nH]ccc3cn1)N=C(N)C21CCC1] |
| B212 | 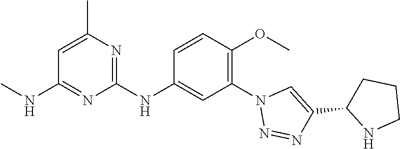 [SMILES: CNc1cc(C)nc(Nc2ccc(OC)c(-n3cc([C@@H]4CCCN4)nn3)c2)n1] |
| B213 | 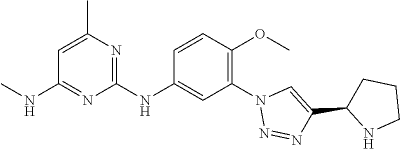 [SMILES: CNc1cc(C)nc(Nc2ccc(OC)c(-n3cc([C@H]4CCCN4)nn3)c2)n1] |
| B214 | 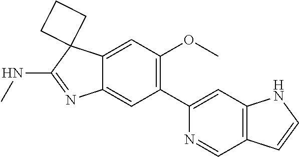 [SMILES: CNC1=Nc2cc(-c3cc4[nH]ccc4cn3)c(OC)cc2C12CCC2] |
| B215 | 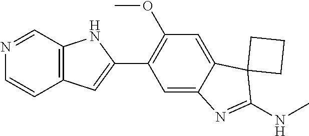 [SMILES: CNC1=Nc2cc(-c3cc4ccncc4[nH]3)c(OC)cc2C12CCC2] |
| B216 | 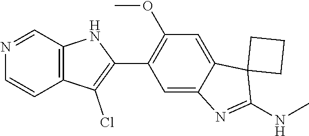 [SMILES: CNC1=Nc2cc(-c3[nH]c4cnccc4c3Cl)c(OC)cc2C12CCC2] |
| B217 | 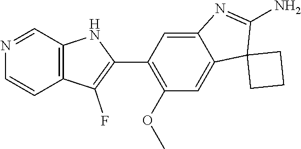 [SMILES: COc1cc2c(cc1-c1[nH]c3cnccc3c1F)N=C(N)C21CCC1] |
| B218 | 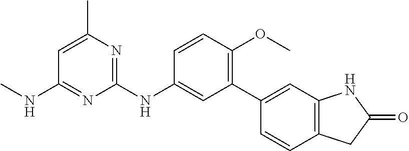 [SMILES: CNc1cc(C)nc(Nc2ccc(OC)c(-c3ccc4c(c3)NC(=O)C4)c2)n1] |
| B219 | 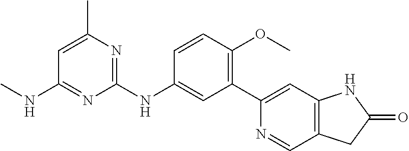 [SMILES: CNc1cc(C)nc(Nc2ccc(OC)c(-c3cc4c(cn3)CC(=O)N4)c2)n1] |
| B220 | 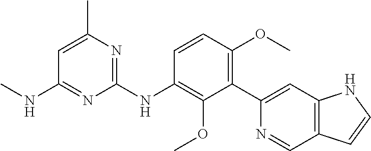 [SMILES: CNc1cc(C)nc(Nc2ccc(OC)c(-c3cc4[nH]ccc4cn3)c2OC)n1] |
| B221 | 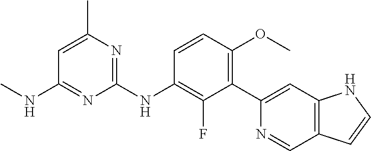 [SMILES: CNc1cc(C)nc(Nc2ccc(OC)c(-c3cc4[nH]ccc4cn3)c2F)n1] |
| B222 | 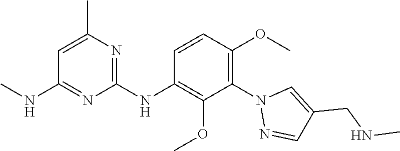 |
| B223 | 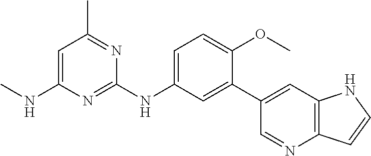 [SMILES: CNc1cc(C)nc(Nc2ccc(OC)c(-c3cnc4cc[nH]c4c3)c2)n1] |
| B224 | 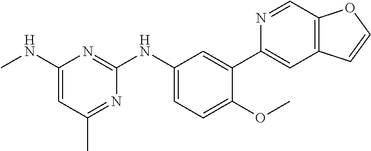 [SMILES: CNc1cc(C)nc(Nc2ccc(OC)c(-c3cc4ccoc4cn3)c2)n1] |
| B225 | 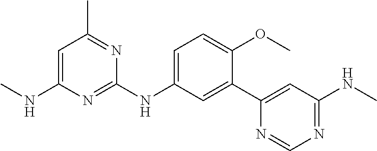 |
| B226 | 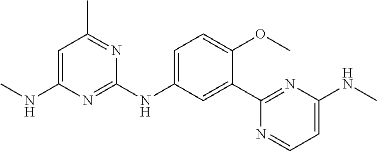 |
| B227 | 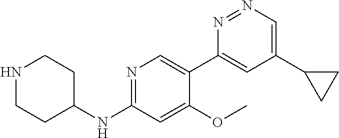 [SMILES: COc1cc(NC2CCNCC2)ncc1-c1cc(C2CC2)cnn1] |
| B228 | 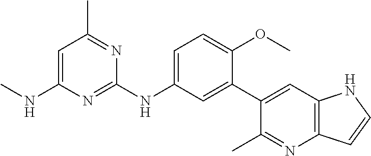 [SMILES: CNc1cc(C)nc(Nc2ccc(OC)c(-c3cc4[nH]ccc4nc3C)c2)n1] |
| B229 | 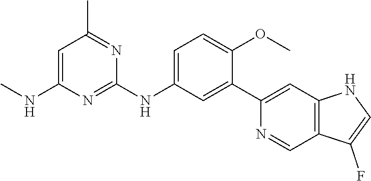 [SMILES: CNc1cc(C)nc(Nc2ccc(OC)c(-c3cc4[nH]cc(F)c4cn3)c2)n1] |
| B230 | 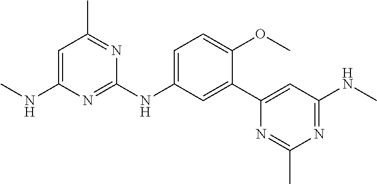 |
| B231 | 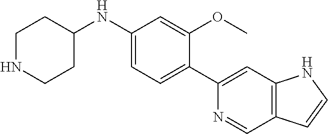 [SMILES: COc1cc(NC2CCNCC2)ccc1-c1cc2[nH]ccc2cn1] |
| B232 | 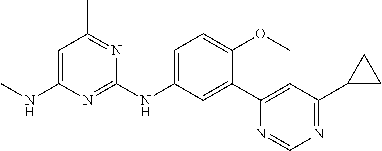 [SMILES: CNc1cc(C)nc(Nc2ccc(OC)c(-c3cc(C4CC4)ncn3)c2)n1] |
| B233 | 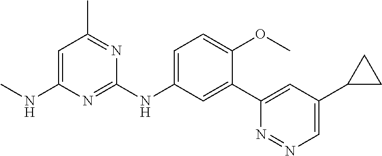 [SMILES: CNc1cc(C)nc(Nc2ccc(OC)c(-c3cc(C4CC4)cnn3)c2)n1] |
| B234 | 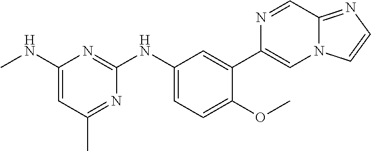 [SMILES: CNc1cc(C)nc(Nc2ccc(OC)c(-c3cn4ccnc4cn3)c2)n1] |
| B235 | 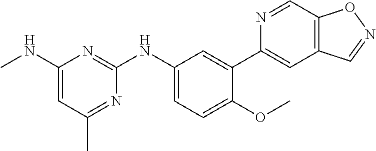 [SMILES: CNc1cc(C)nc(Nc2ccc(OC)c(-c3cc4cnoc4cn3)c2)n1] |
| B236 | 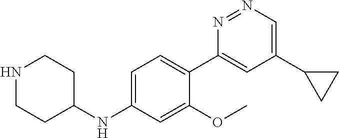 [SMILES: COc1cc(NC2CCNCC2)ccc1-c1cc(C2CC2)cnn1] |
| B237 | 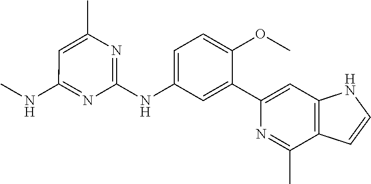 [SMILES: CNc1cc(C)nc(Nc2ccc(OC)c(-c3cc4[nH]ccc4c(C)n3)c2)n1] |
| B238 | 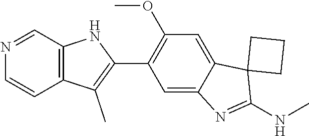 [SMILES: CNC1=Nc2cc(-c3[nH]c4cnccc4c3C)c(OC)cc2C12CCC2] |
| B239 | 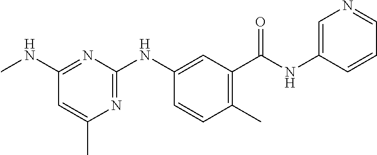 [SMILES: CNc1cc(C)nc(Nc2ccc(C)c(C(=O)Nc3cccnc3)c2)n1] |
| B240 | 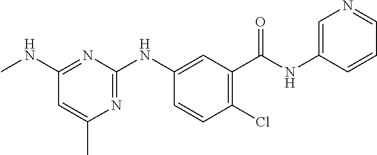 [SMILES: CNc1cc(C)nc(Nc2ccc(Cl)c(C(=O)Nc3cccnc3)c2)n1] |
| B241 | 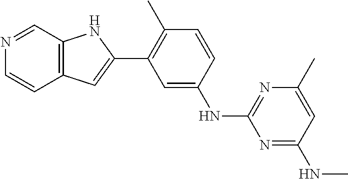 [SMILES: CNc1cc(C)nc(Nc2ccc(C)c(-c3cc4ccncc4[nH]3)c2)n1] |
| B242 | 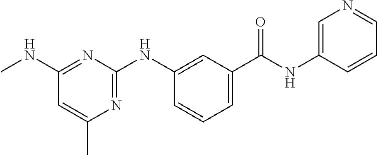 [SMILES: CNc1cc(C)nc(Nc2cccc(C(=O)Nc3cccnc3)c2)n1] |
| B243 | 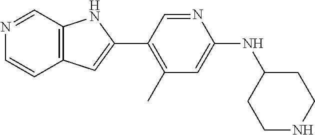 [SMILES: Cc1cc(NC2CCNCC2)ncc1-c1cc2ccncc2[nH]1] |
| B244 | 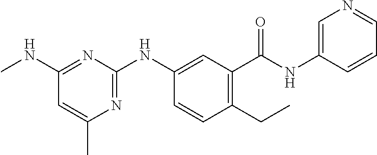 [SMILES: CCc1ccc(Nc2nc(C)cc(NC)n2)cc1C(=O)Nc1cccnc1] |
| B245 | 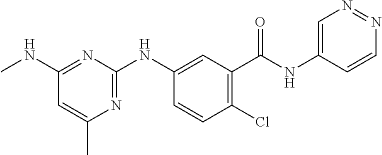 [SMILES: CNc1cc(C)nc(Nc2ccc(Cl)c(C(=O)Nc3ccnnc3)c2)n1] |
| B246 | 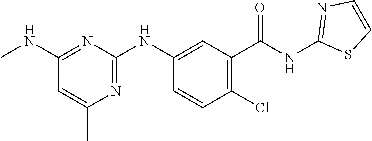 [SMILES: CNc1cc(C)nc(Nc2ccc(Cl)c(C(=O)Nc3nccs3)c2)n1] |
| B247 | 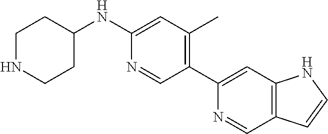 [SMILES: Cc1cc(NC2CCNCC2)ncc1-c1cc2[nH]ccc2cn1] |
| B248 | 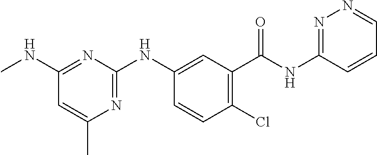 [SMILES: CNc1cc(C)nc(Nc2ccc(Cl)c(C(=O)Nc3cccnn3)c2)n1] |
| B249 | 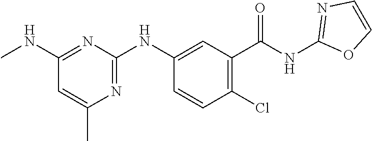 [SMILES: CNc1cc(C)nc(Nc2ccc(Cl)c(C(=O)Nc3ncco3)c2)n1] |
| B250 | 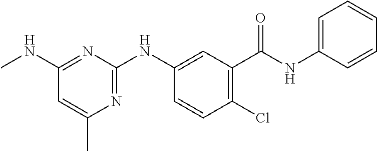 [SMILES: CNc1cc(C)nc(Nc2ccc(Cl)c(C(=O)Nc3ccccc3)c2)n1] |
| B251 | 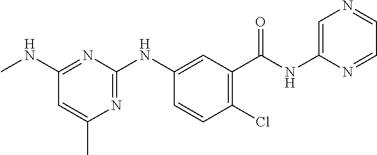 [SMILES: CNc1cc(C)nc(Nc2ccc(Cl)c(C(=O)Nc3cnccn3)c2)n1] |
| B252 | 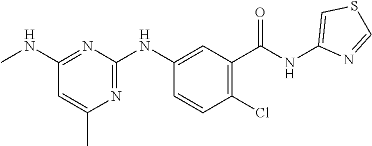 [SMILES: CNc1cc(C)nc(Nc2ccc(Cl)c(C(=O)Nc3cscn3)c2)n1] |
| B253 | 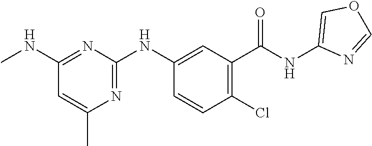 [SMILES: CNc1cc(C)nc(Nc2ccc(Cl)c(C(=O)Nc3cocn3)c2)n1] |
| B254 | 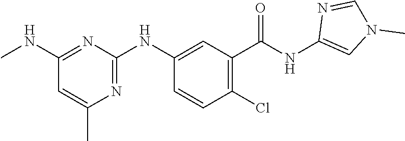 |
| B255 | 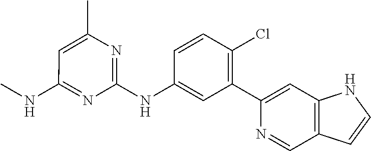 [SMILES: CNc1cc(C)nc(Nc2ccc(Cl)c(-c3cc4[nH]ccc4cn3)c2)n1] |
| B256 | 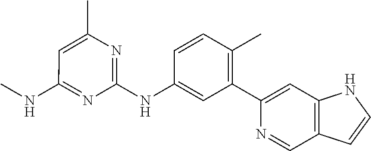 [SMILES: CNc1cc(C)nc(Nc2ccc(C)c(-c3cc4[nH]ccc4cn3)c2)n1] |
| B257 | 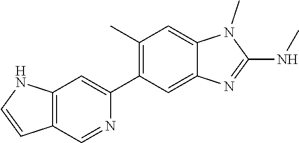 [SMILES: CNc1nc2cc(-c3cc4[nH]ccc4cn3)c(C)cc2n1C] |
| B258 | 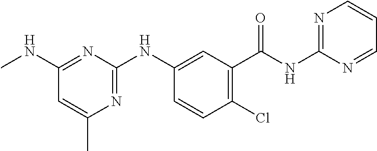 [SMILES: CNc1cc(C)nc(Nc2ccc(Cl)c(C(=O)Nc3ncccn3)c2)n1] |
| B259 | 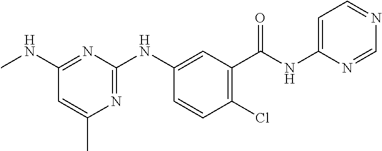 [SMILES: CNc1cc(C)nc(Nc2ccc(Cl)c(C(=O)Nc3ccncn3)c2)n1] |
| B260 | 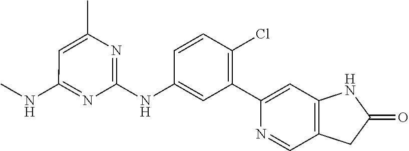 [SMILES: CNc1cc(C)nc(Nc2ccc(Cl)c(-c3cc4c(cn3)CC(=O)N4)c2)n1] |
| B261 | 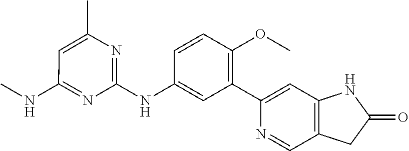 [SMILES: CNc1cc(C)nc(Nc2ccc(OC)c(-c3cc4c(cn3)CC(=O)N4)c2)n1] |
| B262 | 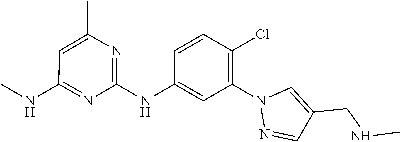 |
| B269 | 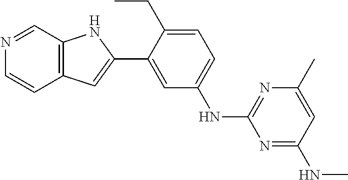 [SMILES: CCc1ccc(Nc2nc(C)cc(NC)n2)cc1-c1cc2ccncc2[nH]1] |
| B271 | 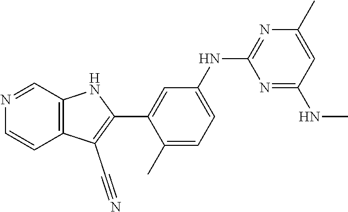 [SMILES: CNc1cc(C)nc(Nc2ccc(C)c(-c3[nH]c4cnccc4c3C#N)c2)n1] |
| B274 | 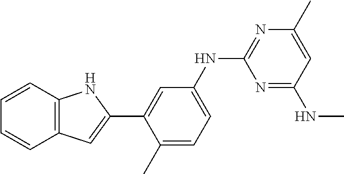 [SMILES: CNc1cc(C)nc(Nc2ccc(C)c(-c3cc4ccccc4[nH]3)c2)n1] |
| B276 | 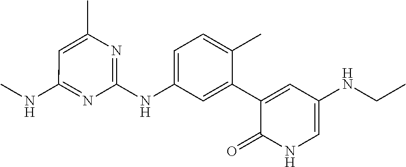 [SMILES: CCNc1c[nH]c(=O)c(-c2cc(Nc3nc(C)cc(NC)n3)ccc2C)c1] |
| B277 | 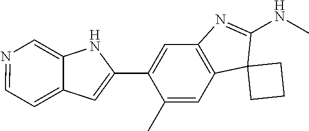 [SMILES: CNC1=Nc2cc(-c3cc4ccncc4[nH]3)c(C)cc2C12CCC2] |
| B278 | 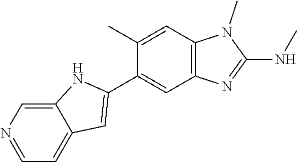 [SMILES: CNc1nc2cc(-c3cc4ccncc4[nH]3)c(C)cc2n1C] |
| B279 | 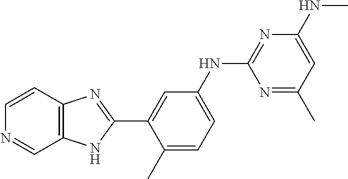 [SMILES: CNc1cc(C)nc(Nc2ccc(C)c(-c3nc4ccncc4[nH]3)c2)n1] |
| B280 | 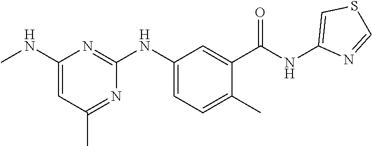 [SMILES: CNc1cc(C)nc(Nc2ccc(C)c(C(=O)Nc3cscn3)c2)n1] |
| B281 | 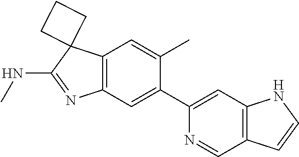 [SMILES: CNC1=Nc2cc(-c3cc4[nH]ccc4cn3)c(C)cc2C12CCC2] |
| B282 | 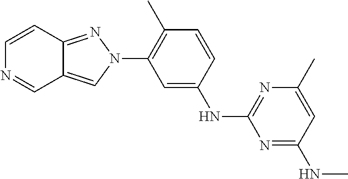 [SMILES: CNc1cc(C)nc(Nc2ccc(C)c(-n3cc4cnccc4n3)c2)n1] |
| B283 | 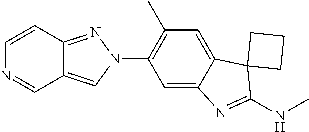 [SMILES: CNC1=Nc2cc(-n3cc4cnccc4n3)c(C)cc2C12CCC2] |
| B284 | 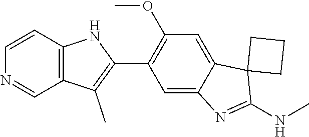 [SMILES: CNC1=Nc2cc(-c3[nH]c4ccncc4c3C)c(OC)cc2C12CCC2] |
| B285 | 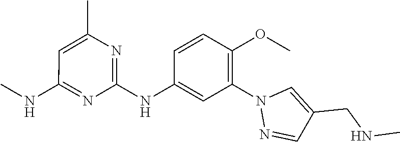 |
| B286 | 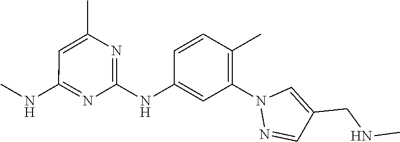 |
| B287 | 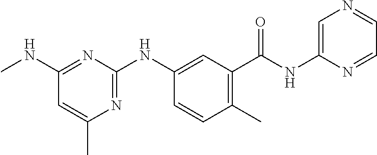 [SMILES: CNc1cc(C)nc(Nc2ccc(C)c(C(=O)Nc3cnccn3)c2)n1] |
| B288 | 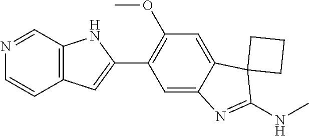 [SMILES: CNC1=Nc2cc(-c3cc4ccncc4[nH]3)c(OC)cc2C12CCC2] |
| B289 | 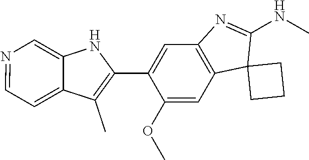 [SMILES: CNC1=Nc2cc(-c3[nH]c4cnccc4c3C)c(OC)cc2C12CCC2] |
| B290 | 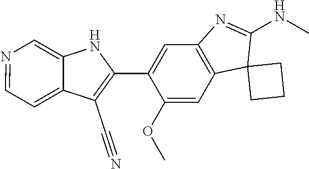 [SMILES: CNC1=Nc2cc(-c3[nH]c4cnccc4c3C#N)c(OC)cc2C12CCC2] |
| B291 | 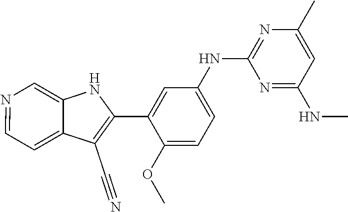 [SMILES: CNc1cc(C)nc(Nc2ccc(OC)c(-c3[nH]c4cnccc4c3C#N)c2)n1] |

The compounds of Table 3 are the compounds found in U.S. Application Nos. 62/436,139 and 62/517,840, and PCT Application No. PCT/US20170067192, the entire contents of which are incorporated herein by reference.

TABLE 4

| Compound | |
|---|---|
| No. | Structure |
| C1 | 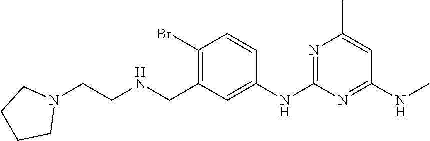 [SMILES: CNc1cc(C)nc(Nc2ccc(Br)c(CNCCN3CCCC3)c2)n1] |
| C2 | 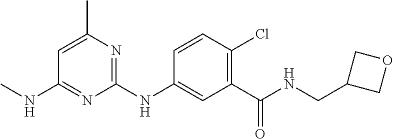 [SMILES: CNc1cc(C)nc(Nc2ccc(Cl)c(C(=O)NCC3COC3)c2)n1] |
| C3 | 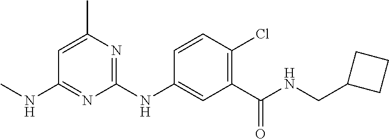 [SMILES: CNc1cc(C)nc(Nc2ccc(Cl)c(C(=O)NCC3CCC3)c2)n1] |
| C4 | 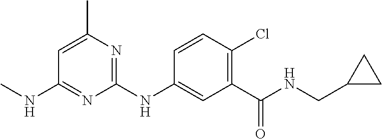 [SMILES: CNc1cc(C)nc(Nc2ccc(Cl)c(C(=O)NCC3CC3)c2)n1] |
| C5 | 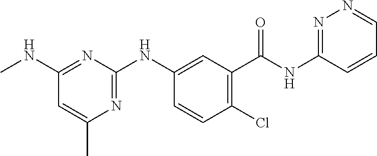 [SMILES: CNc1cc(C)nc(Nc2ccc(Cl)c(C(=O)Nc3cccnn3)c2)n1] |
| C6 | 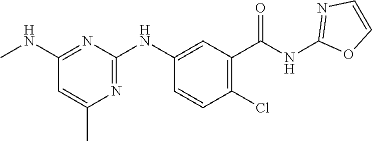 [SMILES: CNc1cc(C)nc(Nc2ccc(Cl)c(C(=O)Nc3ncco3)c2)n1] |
| C7 | 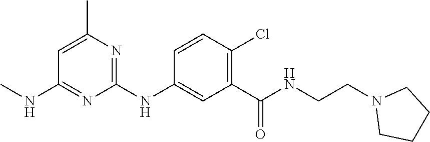 [SMILES: CNc1cc(C)nc(Nc2ccc(Cl)c(C(=O)NCCN3CCCC3)c2)n1] |
| C8 | 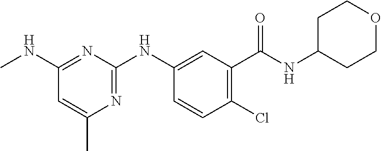 [SMILES: CNc1cc(C)nc(Nc2ccc(Cl)c(C(=O)NC3CCOCC3)c2)n1] |
| C9 | 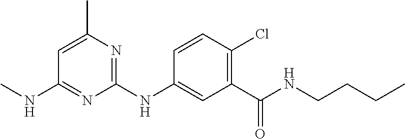 [SMILES: CCCCNC(=O)c1cc(Nc2nc(C)cc(NC)n2)ccc1Cl] |
| C10 | 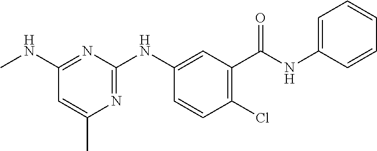 [SMILES: CNc1cc(C)nc(Nc2ccc(Cl)c(C(=O)Nc3ccccc3)c2)n1] |
| C11 | 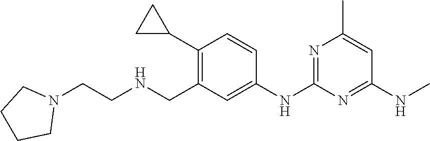 [SMILES: CNc1cc(C)nc(Nc2ccc(C3CC3)c(CNCCN3CCCC3)c2)n1] |
| C12 | 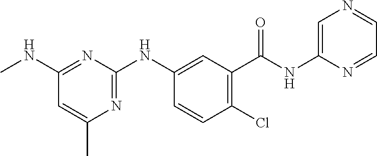 [SMILES: CNc1cc(C)nc(Nc2ccc(Cl)c(C(=O)Nc3cnccn3)c2)n1] |
| C13 | 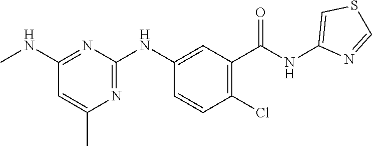 [SMILES: CNc1cc(C)nc(Nc2ccc(Cl)c(C(=O)Nc3cscn3)c2)n1] |
| C14 | 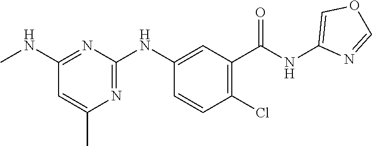 [SMILES: CNc1cc(C)nc(Nc2ccc(Cl)c(C(=O)Nc3cocn3)c2)n1] |
| C15 | 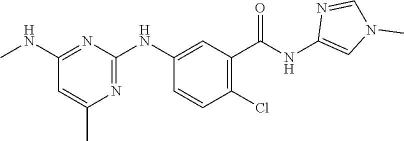 |
| C16 | 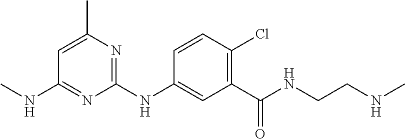 |
| C17 | 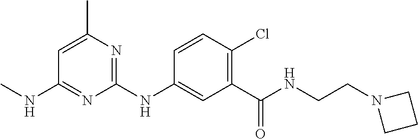 [SMILES: CNc1cc(C)nc(Nc2ccc(Cl)c(C(=O)NCCN3CCC3)c2)n1] |
| C18 | 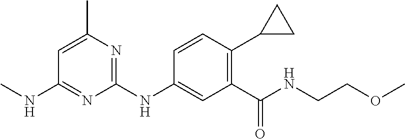 |
| C19 | 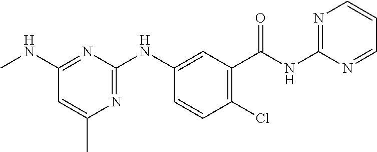 [SMILES: CNc1cc(C)nc(Nc2ccc(Cl)c(C(=O)Nc3ncccn3)c2)n1] |
| C20 | 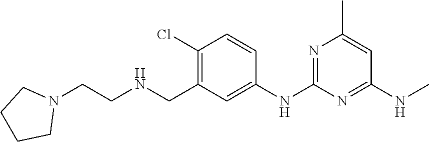 [SMILES: CNc1cc(C)nc(Nc2ccc(Cl)c(CNCCN3CCCC3)c2)n1] |
| C21 | 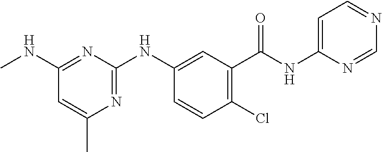 [SMILES: CNc1cc(C)nc(Nc2ccc(Cl)c(C(=O)Nc3ccncn3)c2)n1] |
| C22 | 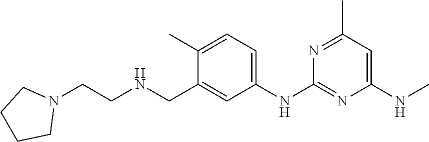 [SMILES: CNc1cc(C)nc(Nc2ccc(C)c(CNCCN3CCCC3)c2)n1] |
| C23 | 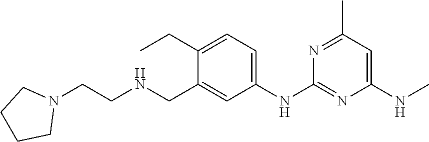 [SMILES: CCc1ccc(Nc2nc(C)cc(NC)n2)cc1CNCCN1CCCC1] |
| C24 | 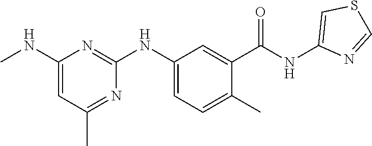 [SMILES: CNc1cc(C)nc(Nc2ccc(C)c(C(=O)Nc3cscn3)c2)n1] |
| C25 | 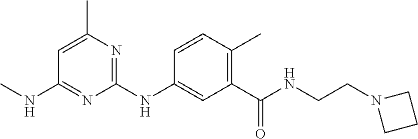 [SMILES: CNc1cc(C)nc(Nc2ccc(C)c(C(=O)NCCN3CCC3)c2)n1] |
| C26 | 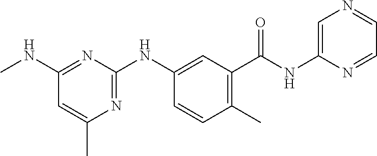 [SMILES: CNc1cc(C)nc(Nc2ccc(C)c(C(=O)Nc3cnccn3)c2)n1] |
| C27 | 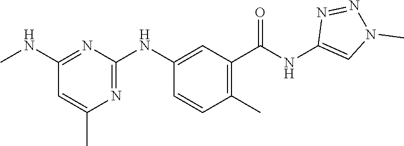 |
| C28 | 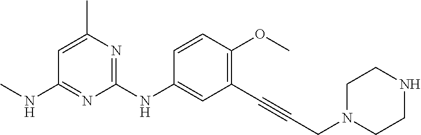 [SMILES: CNc1cc(C)nc(Nc2ccc(OC)c(C#CCN3CCNCC3)c2)n1] |
| C29 | 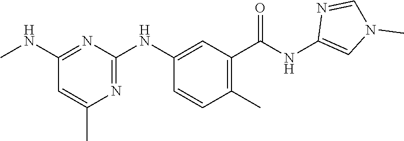 |
| C30 | 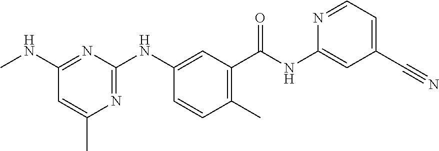 [SMILES: CNc1cc(C)nc(Nc2ccc(C)c(C(=O)Nc3cc(C#N)ccn3)c2)n1] |
| C31 | 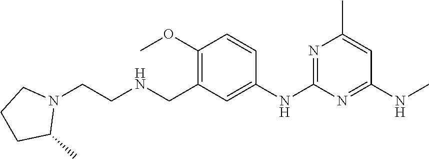 [SMILES: CNc1cc(C)nc(Nc2ccc(OC)c(CNCCN3CCC[C@H]3C)c2)n1] |
| C32 | 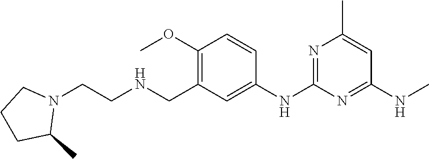 [SMILES: CNc1cc(C)nc(Nc2ccc(OC)c(CNCCN3CCC[C@@H]3C)c2)n1] |
| C33 | 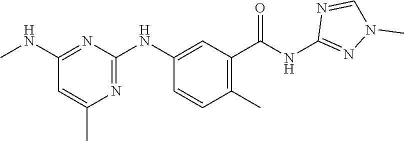 |
| C34 | 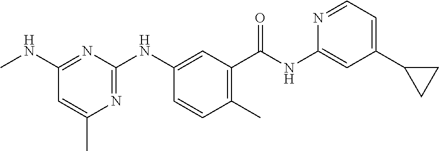 [SMILES: CNc1cc(C)nc(Nc2ccc(C)c(C(=O)Nc3cc(C4CC4)ccn3)c2)n1] |
| C35 | 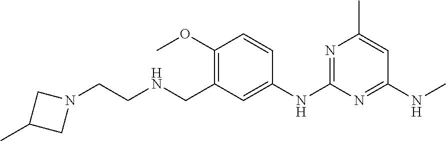 |
| C36 | 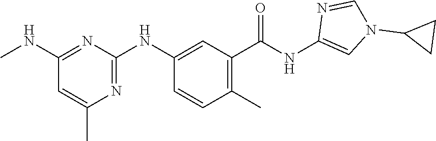 [SMILES: CNc1cc(C)nc(Nc2ccc(C)c(C(=O)Nc3cn(C4CC4)cn3)c2)n1] |
| C37 | 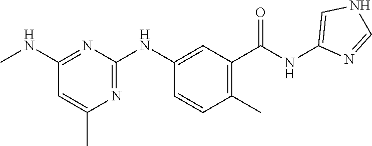 [SMILES: CNc1cc(C)nc(Nc2ccc(C)c(C(=O)Nc3c[nH]cn3)c2)n1] |
| C38 | 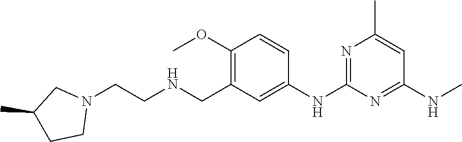 |
| C39 | 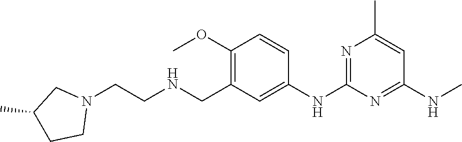 |
| C40 | 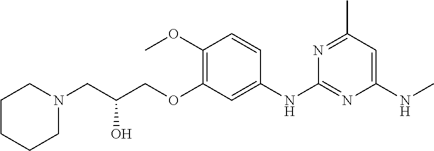 [SMILES: CNc1cc(C)nc(Nc2ccc(OC)c(OC[C@H](O)CN3CCCCC3)c2)n1] |
| C41 | 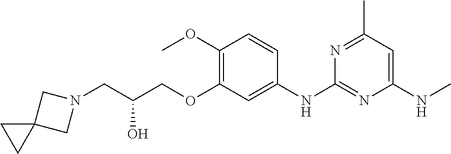 [SMILES: CNc1cc(C)nc(Nc2ccc(OC)c(OC[C@H](O)CN3CC4(CC4)C3)c2)n1] |
| C42 | 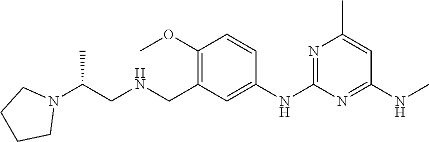 [SMILES: CNc1cc(C)nc(Nc2ccc(OC)c(CNC[C@@H](C)N3CCCC3)c2)n1] |
| C43 | 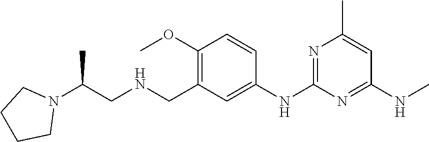 [SMILES: CNc1cc(C)nc(Nc2ccc(OC)c(CNC[C@H](C)N3CCCC3)c2)n1] |
| C44 | 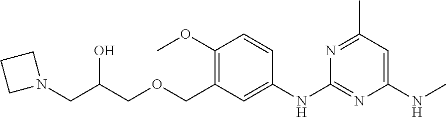 [SMILES: CNc1cc(C)nc(Nc2ccc(OC)c(COCC(O)CN3CCC3)c2)n1] |
| C45 | 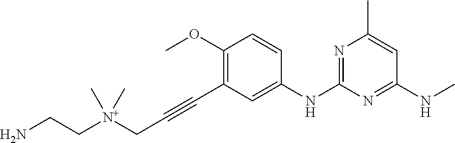 [SMILES: CNc1cc(C)nc(Nc2ccc(OC)c(C#CC[N+](C)(C)CCN)c2)n1] |
| C46 | 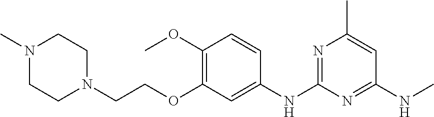 |
| C47 | 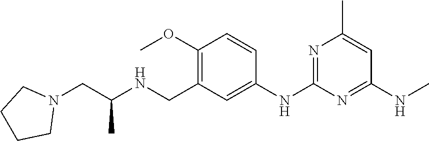 [SMILES: CNc1cc(C)nc(Nc2ccc(OC)c(CN[C@@H](C)CN3CCCC3)c2)n1] |
| C48 | 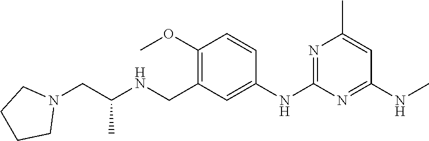 [SMILES: CNc1cc(C)nc(Nc2ccc(OC)c(CN[C@H](C)CN3CCCC3)c2)n1] |
| C49 | 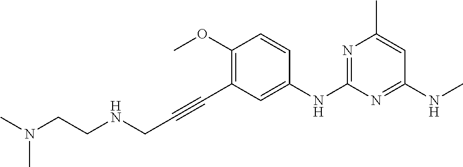 |
| C50 | 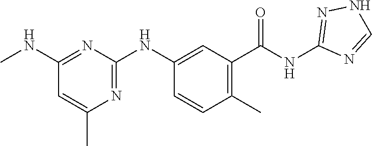 [SMILES: CNc1cc(C)nc(Nc2ccc(C)c(C(=O)Nc3nc[nH]n3)c2)n1] |
| C51 | 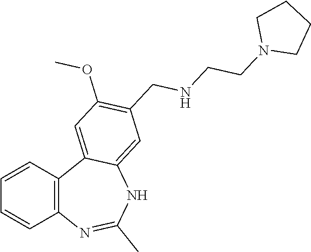 [SMILES: COc1cc2c(cc1CNCCN1CCCC1)NC(C)=Nc1ccccc1-2] |
| C52 | 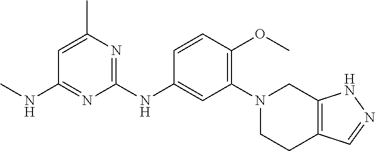 [SMILES: CNc1cc(C)nc(Nc2ccc(OC)c(N3CCc4cn[nH]c4C3)c2)n1] |
| C53 | 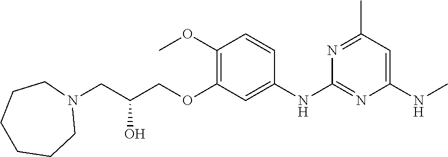 [SMILES: CNc1cc(C)nc(Nc2ccc(OC)c(OC[C@H](O)CN3CCCCCC3)c2)n1] |
| C54 | 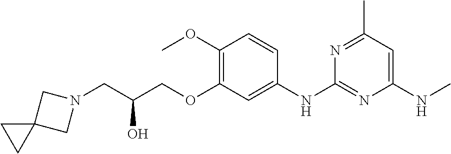 [SMILES: CNc1cc(C)nc(Nc2ccc(OC)c(OC[C@@H](O)CN3CC4(CC4)C3)c2)n1] |
| C55 | 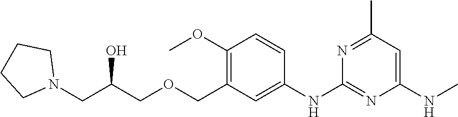 [SMILES: CNc1cc(C)nc(Nc2ccc(OC)c(COC[C@H](O)CN3CCCC3)c2)n1] |
| C56 | 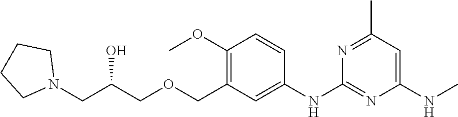 [SMILES: CNc1cc(C)nc(Nc2ccc(OC)c(COC[C@@H](O)CN3CCCC3)c2)n1] |
| C57 | 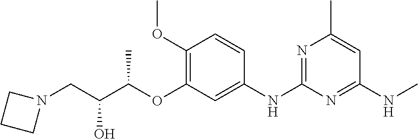 [SMILES: CNc1cc(C)nc(Nc2ccc(OC)c(O[C@@H](C)[C@H](O)CN3CCC3)c2)n1] |
| C58 | 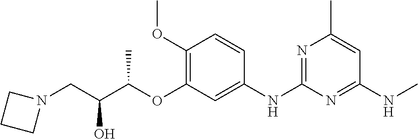 [SMILES: CNc1cc(C)nc(Nc2ccc(OC)c(O[C@@H](C)[C@@H](O)CN3CCC3)c2)n1] |
| C59 | 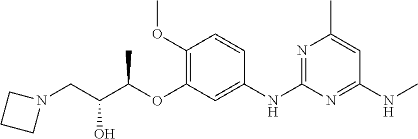 [SMILES: CNc1cc(C)nc(Nc2ccc(OC)c(O[C@H](C)[C@H](O)CN3CCC3)c2)n1] |
| C60 | 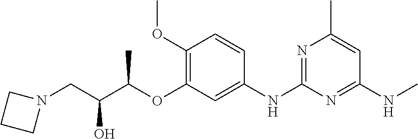 [SMILES: CNc1cc(C)nc(Nc2ccc(OC)c(O[C@H](C)[C@@H](O)CN3CCC3)c2)n1] |
| C61 | 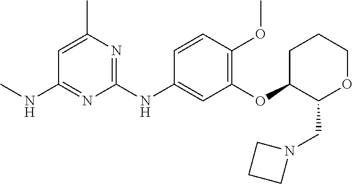 [SMILES: CNc1cc(C)nc(Nc2ccc(OC)c(O[C@H]3CCCO[C@@H]3CN3CCC3)c2)n1] |
| C62 | 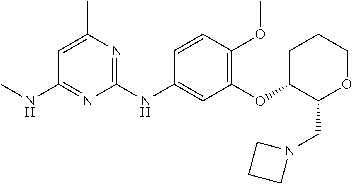 [SMILES: CNc1cc(C)nc(Nc2ccc(OC)c(O[C@@H]3CCCO[C@@H]3CN3CCC3)c2)n1] |
| C63 | 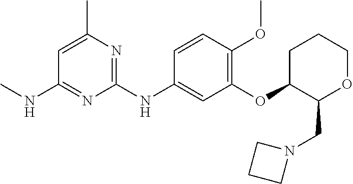 [SMILES: CNc1cc(C)nc(Nc2ccc(OC)c(O[C@H]3CCCO[C@H]3CN3CCC3)c2)n1] |
| C64 | 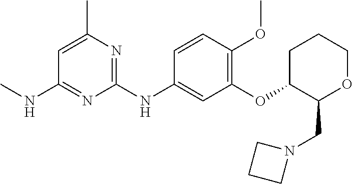 [SMILES: CNc1cc(C)nc(Nc2ccc(OC)c(O[C@@H]3CCCO[C@H]3CN3CCC3)c2)n1] |
| C65 | 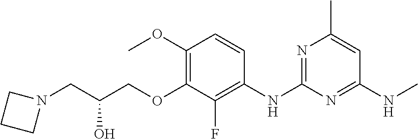 [SMILES: CNc1cc(C)nc(Nc2ccc(OC)c(OC[C@H](O)CN3CCC3)c2F)n1] |
| C66 | 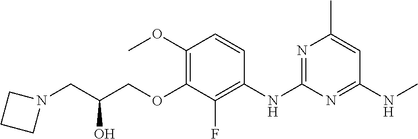 [SMILES: CNc1cc(C)nc(Nc2ccc(OC)c(OC[C@@H](O)CN3CCC3)c2F)n1] |
| C67 | 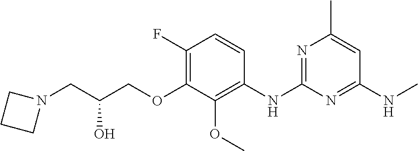 [SMILES: CNc1cc(C)nc(Nc2ccc(F)c(OC[C@H](O)CN3CCC3)c2OC)n1] |
| C68 | 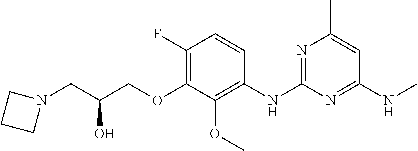 [SMILES: CNc1cc(C)nc(Nc2ccc(F)c(OC[C@@H](O)CN3CCC3)c2OC)n1] |
| C69 | 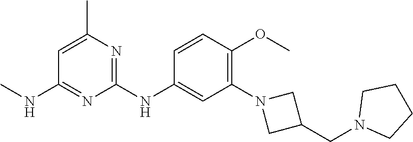 [SMILES: CNc1cc(C)nc(Nc2ccc(OC)c(N3CC(CN4CCCC4)C3)c2)n1] |
| C70 | 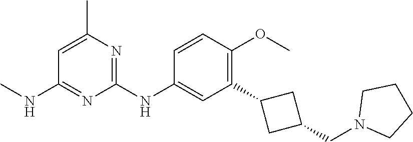 [SMILES: CNc1cc(C)nc(Nc2ccc(OC)c([C@H]3C[C@@H](CN4CCCC4)C3)c2)n1] |
| C71 | 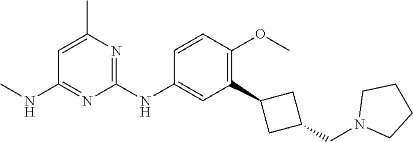 [SMILES: CNc1cc(C)nc(Nc2ccc(OC)c([C@H]3C[C@H](CN4CCCC4)C3)c2)n1] |
| C72 | 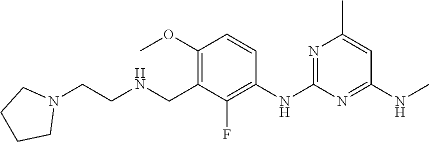 [SMILES: CNc1cc(C)nc(Nc2ccc(OC)c(CNCCN3CCCC3)c2F)n1] |
| C73 | 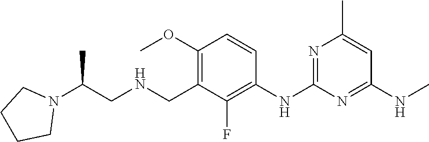 [SMILES: CNc1cc(C)nc(Nc2ccc(OC)c(CNC[C@H](C)N3CCCC3)c2F)n1] |
| C74 | 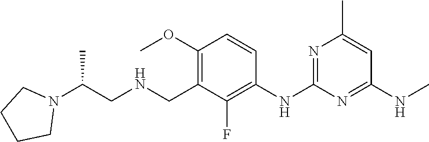 [SMILES: CNc1cc(C)nc(Nc2ccc(OC)c(CNC[C@@H](C)N3CCCC3)c2F)n1] |
| C75 | 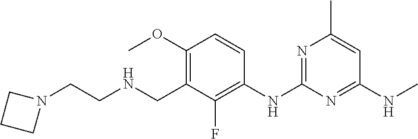 [SMILES: CNc1cc(C)nc(Nc2ccc(OC)c(CNCCN3CCC3)c2F)n1] |
| C76 | 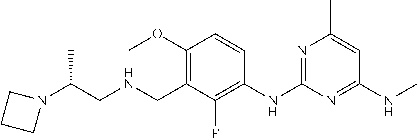 [SMILES: CNc1cc(C)nc(Nc2ccc(OC)c(CNC[C@@H](C)N3CCC3)c2F)n1] |
| C77 | 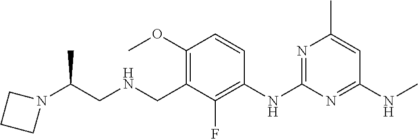 [SMILES: CNc1cc(C)nc(Nc2ccc(OC)c(CNC[C@H](C)N3CCC3)c2F)n1] |
| C78 | 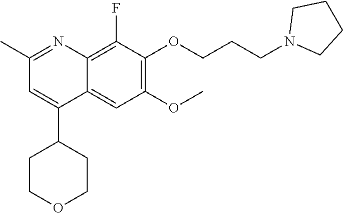 [SMILES: COc1cc2c(C3CCOCC3)cc(C)nc2c(F)c1OCCCN1CCCC1] |
| C79 | 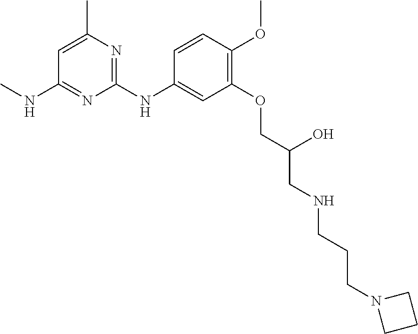 [SMILES: CNc1cc(C)nc(Nc2ccc(OC)c(OCC(O)CNCCCN3CCC3)c2)n1] |
| C79S | 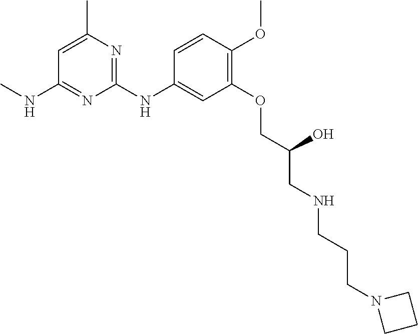 [SMILES: CNc1cc(C)nc(Nc2ccc(OC)c(OC[C@@H](O)CNCCCN3CCC3)c2)n1] |
| C79R | 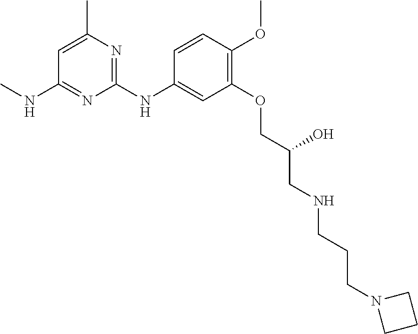 [SMILES: CNc1cc(C)nc(Nc2ccc(OC)c(OC[C@H](O)CNCCCN3CCC3)c2)n1] |
| C80 | 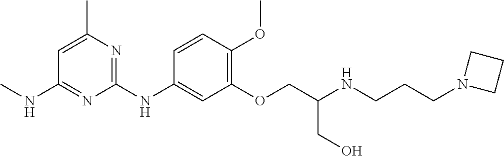 [SMILES: CNc1cc(C)nc(Nc2ccc(OC)c(OCC(CO)NCCCN3CCC3)c2)n1] |
| C80S | 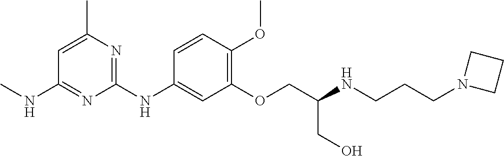 [SMILES: CNc1cc(C)nc(Nc2ccc(OC)c(OC[C@H](CO)NCCCN3CCC3)c2)n1] |
| C80R | 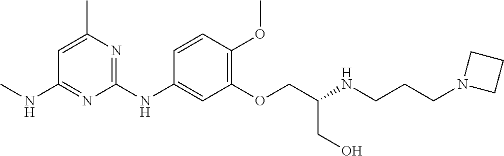 [SMILES: CNc1cc(C)nc(Nc2ccc(OC)c(OC[C@@H](CO)NCCCN3CCC3)c2)n1] |

The compounds of Table 4 are the compounds found in U.S. Application No. 62/573,442 and 62/746,495, and PCT Application No. PCT/US2018/056333, the entire contents of which are incorporated herein by reference.

TABLE 4A

| Cmpd. | |
|---|---|
| No. | Structure |
| CA1 | 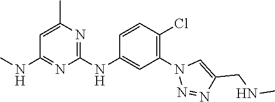 |
| CA2 | 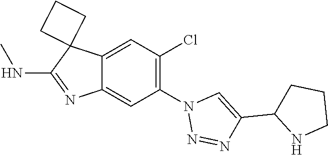 [SMILES: CNC1=Nc2cc(-n3cc(C4CCCN4)nn3)c(Cl)cc2C12CCC2] |
| CA2S | 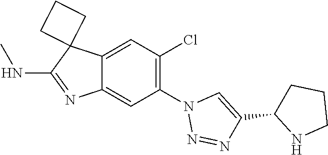 [SMILES: CNC1=Nc2cc(-n3cc([C@@H]4CCCN4)nn3)c(Cl)cc2C12CCC2] |
| CA2R | 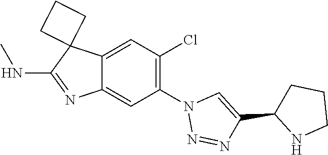 [SMILES: CNC1=Nc2cc(-n3cc([C@H]4CCCN4)nn3)c(Cl)cc2C12CCC2] |
| CA3 | 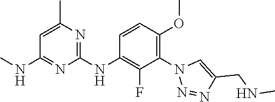 |
| CA4 | 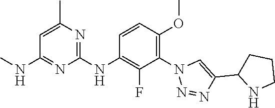 [SMILES: CNc1cc(C)nc(Nc2ccc(OC)c(-n3cc(C4CCCN4)nn3)c2F)n1] |
| CA4S | 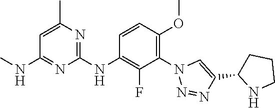 [SMILES: CNc1cc(C)nc(Nc2ccc(OC)c(-n3cc([C@@H]4CCCN4)nn3)c2F)n1] |
| CA4R | 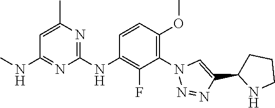 [SMILES: CNc1cc(C)nc(Nc2ccc(OC)c(-n3cc([C@H]4CCCN4)nn3)c2F)n1] |
| CA5 | 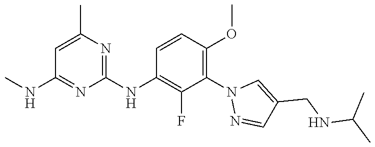 [SMILES: CNc1cc(C)nc(Nc2ccc(OC)c(-n3cc(CNC(C)C)cn3)c2F)n1] |
| CA6 | 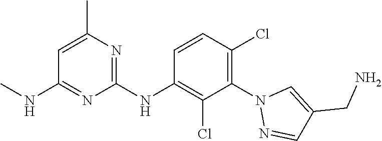 [SMILES: CNc1cc(C)nc(Nc2ccc(Cl)c(-n3cc(CN)cn3)c2Cl)n1] |
| CA7 | 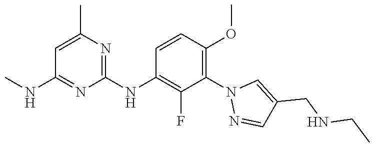 [SMILES: CCNCc1cnn(-c2c(OC)ccc(Nc3nc(C)cc(NC)n3)c2F)c1] |
| CA8 | 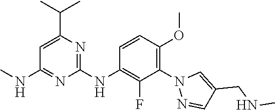 |
| CA9 | 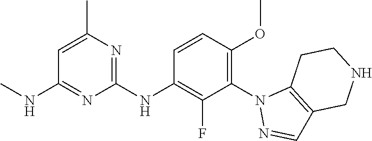 [SMILES: CNc1cc(C)nc(Nc2ccc(OC)c(-n3ncc4c3CCNC4)c2F)n1] |
| CA10 | 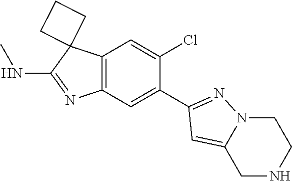 [SMILES: CNC1=Nc2cc(-c3cc4n(n3)CCNC4)c(Cl)cc2C12CCC2] |
| CA11 | 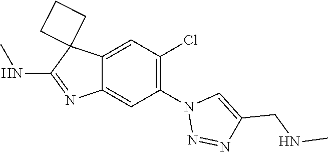 |
| CA12 | 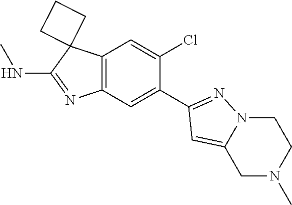 |
| CA13 | 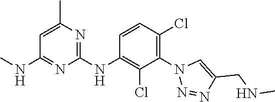 |
| CA14 | 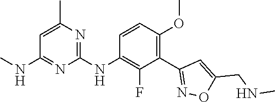 |
| CA15 | 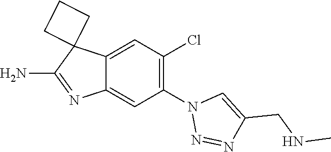 [SMILES: CNCc1cn(-c2cc3c(cc2Cl)C2(CCC2)C(N)=N3)nn1] |
| CA16 | 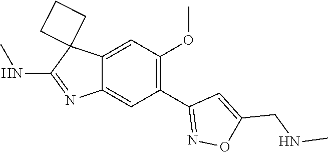 |
| CA17 | 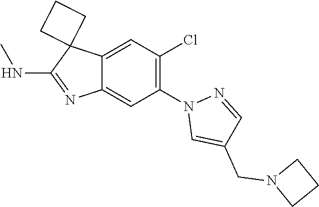 [SMILES: CNC1=Nc2cc(-n3cc(CN4CCC4)cn3)c(Cl)cc2C12CCC2] |
| CA18 | 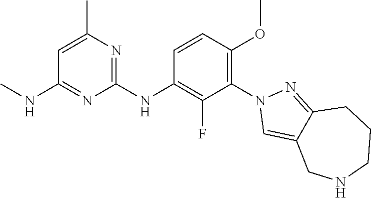 [SMILES: CNc1cc(C)nc(Nc2ccc(OC)c(-n3cc4c(n3)CCCNC4)c2F)n1] |
| CA19 | 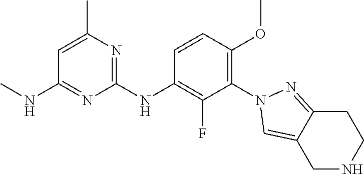 [SMILES: CNc1cc(C)nc(Nc2ccc(OC)c(-n3cc4c(n3)CCNC4)c2F)n1] |
| CA20 | 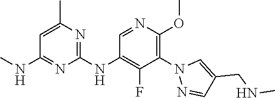 |
| CA21 | 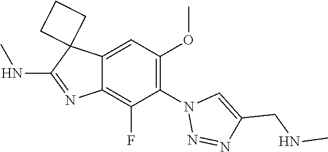 |
| CA22 | 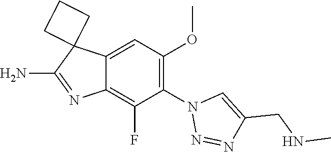 [SMILES: CNCc1cn(-c2c(OC)cc3c(c2F)N=C(N)C32CCC2)nn1] |
| CA23 | 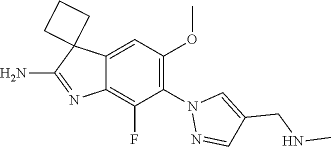 [SMILES: CNCc1cnn(-c2c(OC)cc3c(c2F)N=C(N)C32CCC2)c1] |
| CA24 | 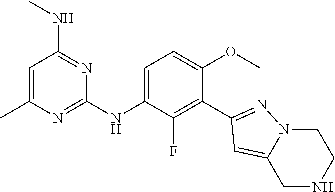 [SMILES: CNc1cc(C)nc(Nc2ccc(OC)c(-c3cc4n(n3)CCNC4)c2F)n1] |
| CA25 | 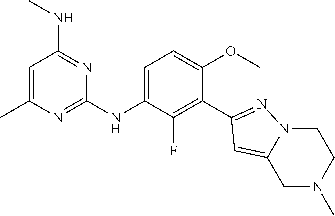 |
| CA26 | 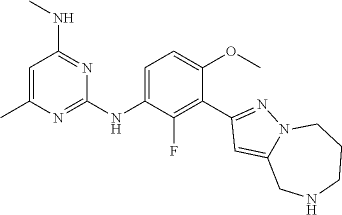 [SMILES: CNc1cc(C)nc(Nc2ccc(OC)c(-c3cc4n(n3)CCCNC4)c2F)n1] |
| CA27 | 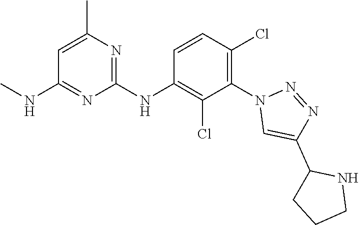 [SMILES: CNc1cc(C)nc(Nc2ccc(Cl)c(-n3cc(C4CCCN4)nn3)c2Cl)n1] |
| CA27R | 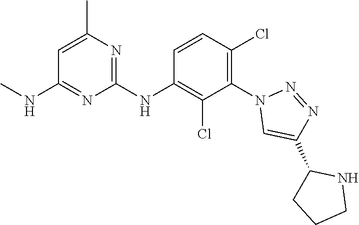 [SMILES: CNc1cc(C)nc(Nc2ccc(Cl)c(-n3cc([C@H]4CCCN4)nn3)c2Cl)n1] |
| CA27S | 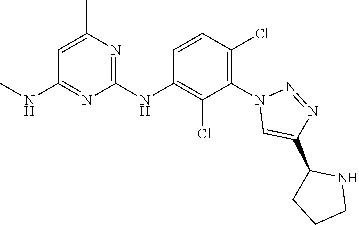 [SMILES: CNc1cc(C)nc(Nc2ccc(Cl)c(-n3cc([C@@H]4CCCN4)nn3)c2Cl)n1] |
| CA28 | 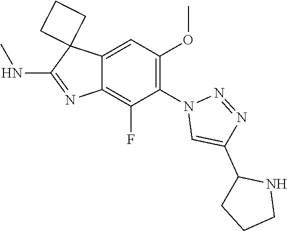 [SMILES: CNC1=Nc2c(cc(OC)c(-n3cc(C4CCCN4)nn3)c2F)C12CCC2] |
| CA28R | 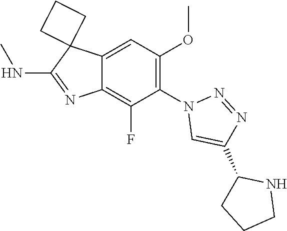 [SMILES: CNC1=Nc2c(cc(OC)c(-n3cc([C@H]4CCCN4)nn3)c2F)C12CCC2] |
| CA28S | 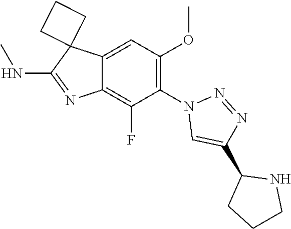 [SMILES: CNC1=Nc2c(cc(OC)c(-n3cc([C@@H]4CCCN4)nn3)c2F)C12CCC2] |
| CA29 | 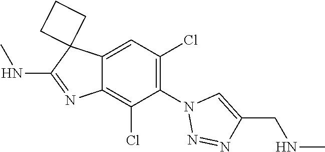 |
| CA30 | 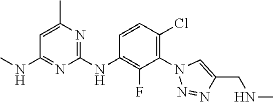 |
| CA31 | 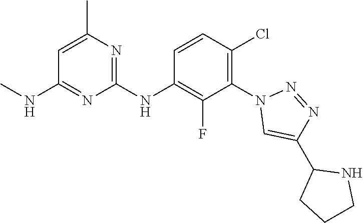 [SMILES: CNc1cc(C)nc(Nc2ccc(Cl)c(-n3cc(C4CCCN4)nn3)c2F)n1] |
| CA31S | 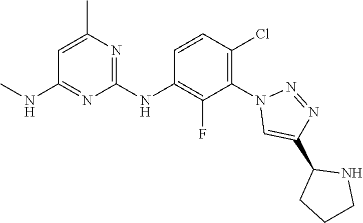 [SMILES: CNc1cc(C)nc(Nc2ccc(Cl)c(-n3cc([C@@H]4CCCN4)nn3)c2F)n1] |
| CA31R | 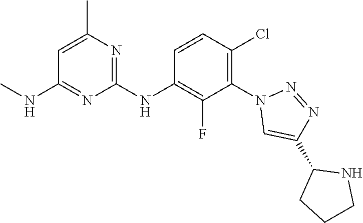 [SMILES: CNc1cc(C)nc(Nc2ccc(Cl)c(-n3cc([C@H]4CCCN4)nn3)c2F)n1] |
| CA32 | 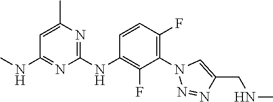 |
| CA33 | 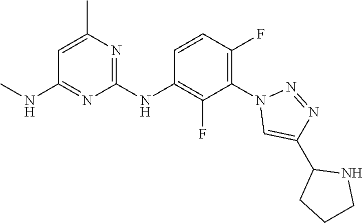 [SMILES: CNc1cc(C)nc(Nc2ccc(F)c(-n3cc(C4CCCN4)nn3)c2F)n1] |
| CA33S | 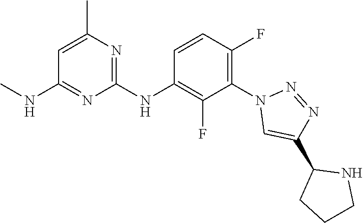 [SMILES: CNc1cc(C)nc(Nc2ccc(F)c(-n3cc([C@@H]4CCCN4)nn3)c2F)n1] |
| CA33R | 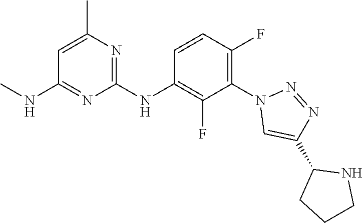 [SMILES: CNc1cc(C)nc(Nc2ccc(F)c(-n3cc([C@H]4CCCN4)nn3)c2F)n1] |
| CA34 | 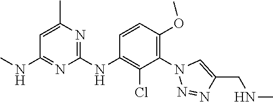 |
| CA35 | 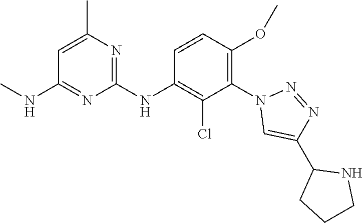 [SMILES: CNc1cc(C)nc(Nc2ccc(OC)c(-n3cc(C4CCCN4)nn3)c2Cl)n1] |
| CA35S | 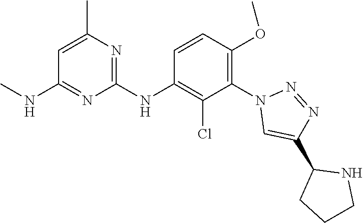 [SMILES: CNc1cc(C)nc(Nc2ccc(OC)c(-n3cc([C@@H]4CCCN4)nn3)c2Cl)n1] |
| CA35R | 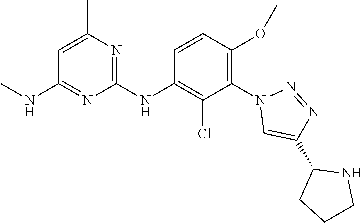 [SMILES: CNc1cc(C)nc(Nc2ccc(OC)c(-n3cc([C@H]4CCCN4)nn3)c2Cl)n1] |
| CA36 | 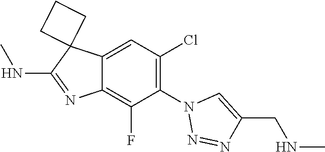 |
| CA37 | 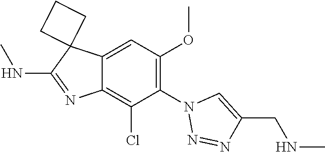 |
| CA38 | 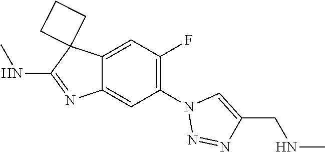 |
| CA39 | 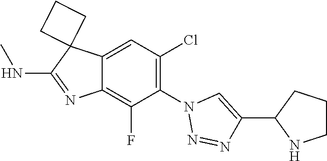 [SMILES: CNC1=Nc2c(cc(Cl)c(-n3cc(C4CCCN4)nn3)c2F)C12CCC2] |
| CA39S | 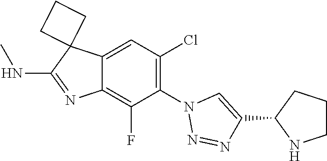 [SMILES: CNC1=Nc2c(cc(Cl)c(-n3cc([C@@H]4CCCN4)nn3)c2F)C12CCC2] |
| CA39R | 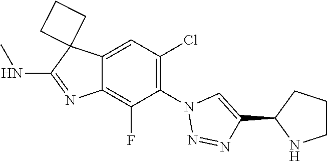 [SMILES: CNC1=Nc2c(cc(Cl)c(-n3cc([C@H]4CCCN4)nn3)c2F)C12CCC2] |
| CA40 | 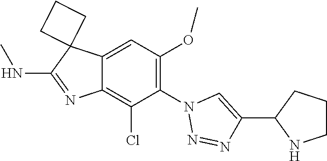 [SMILES: CNC1=Nc2c(cc(OC)c(-n3cc(C4CCCN4)nn3)c2Cl)C12CCC2] |
| CA40S | 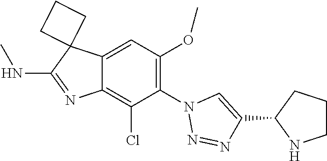 [SMILES: CNC1=Nc2c(cc(OC)c(-n3cc([C@@H]4CCCN4)nn3)c2Cl)C12CCC2] |
| CA40R | 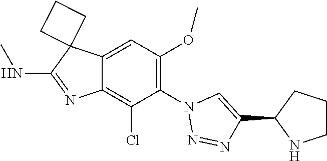 [SMILES: CNC1=Nc2c(cc(OC)c(-n3cc([C@H]4CCCN4)nn3)c2Cl)C12CCC2] |
| CA41 | 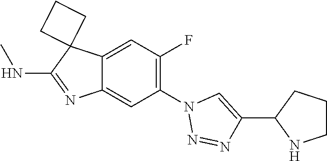 [SMILES: CNC1=Nc2cc(-n3cc(C4CCCN4)nn3)c(F)cc2C12CCC2] |
| CA41S | 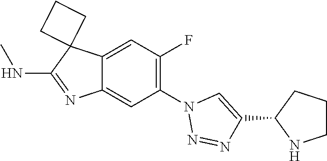 [SMILES: CNC1=Nc2cc(-n3cc([C@@H]4CCCN4)nn3)c(F)cc2C12CCC2] |
| CA41R | 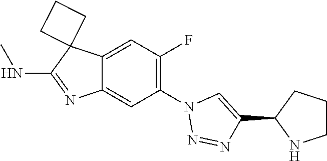 [SMILES: CNC1=Nc2cc(-n3cc([C@H]4CCCN4)nn3)c(F)cc2C12CCC2] |
| CA42 | 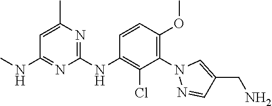 [SMILES: CNc1cc(C)nc(Nc2ccc(OC)c(-n3cc(CN)cn3)c2Cl)n1] |
| CA43 | 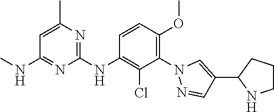 [SMILES: CNc1cc(C)nc(Nc2ccc(OC)c(-n3cc(C4CCCN4)cn3)c2Cl)n1] |
| CA43S | 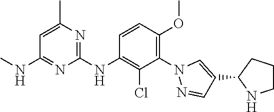 [SMILES: CNc1cc(C)nc(Nc2ccc(OC)c(-n3cc([C@@H]4CCCN4)cn3)c2Cl)n1] |
| CA43R | 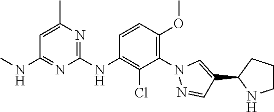 [SMILES: CNc1cc(C)nc(Nc2ccc(OC)c(-n3cc([C@H]4CCCN4)cn3)c2Cl)n1] |
| CA44 | 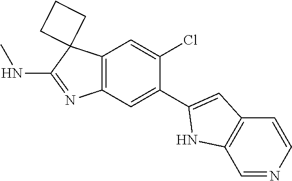 [SMILES: CNC1=Nc2cc(-c3cc4ccncc4[nH]3)c(Cl)cc2C12CCC2] |
| CA45 | 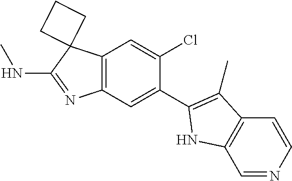 [SMILES: CNC1=Nc2cc(-c3[nH]c4cnccc4c3C)c(Cl)cc2C12CCC2] |
| CA46 | 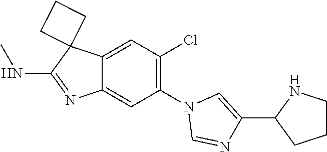 [SMILES: CNC1=Nc2cc(-n3cnc(C4CCCN4)c3)c(Cl)cc2C12CCC2] |
| CA46S | 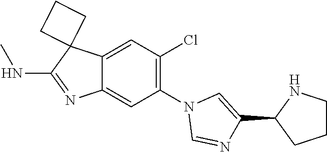 [SMILES: CNC1=Nc2cc(-n3cnc([C@@H]4CCCN4)c3)c(Cl)cc2C12CCC2] |
| CA46R | 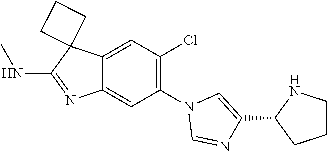 [SMILES: CNC1=Nc2cc(-n3cnc([C@H]4CCCN4)c3)c(Cl)cc2C12CCC2] |
| CA47 | 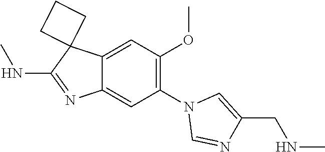 |
| CA48 | 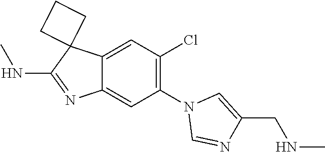 |
| CA49 | 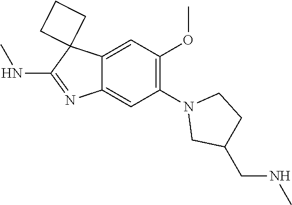 |
| CA50 | 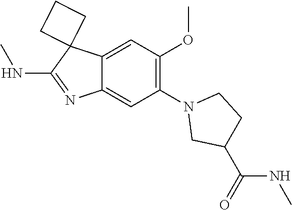 |
| CA51 | 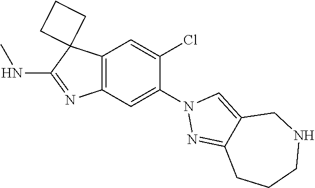 [SMILES: CNC1=Nc2cc(-n3cc4c(n3)CCCNC4)c(Cl)cc2C12CCC2] |
| CA52 | 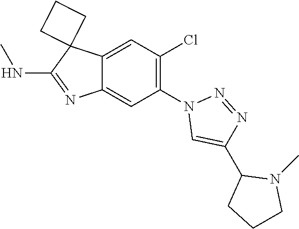 |
| CA52S | 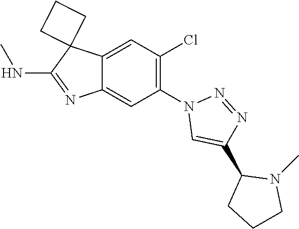 |
| CA52R | 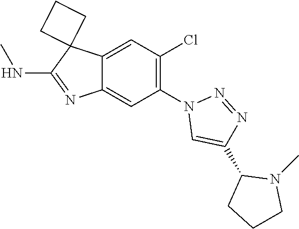 |
| CA53 | 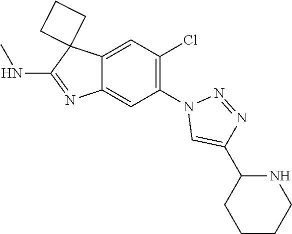 [SMILES: CNC1=Nc2cc(-n3cc(C4CCCCN4)nn3)c(Cl)cc2C12CCC2] |
| CA53S | 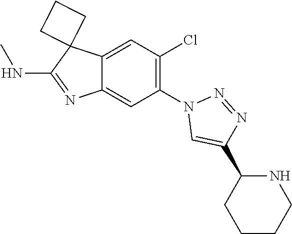 [SMILES: CNC1=Nc2cc(-n3cc([C@@H]4CCCCN4)nn3)c(Cl)cc2C12CCC2] |
| CA53R | 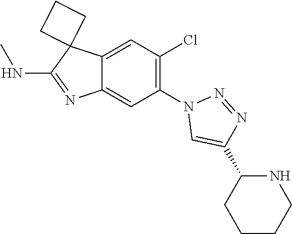 [SMILES: CNC1=Nc2cc(-n3cc([C@H]4CCCCN4)nn3)c(Cl)cc2C12CCC2] |
| CA54 | 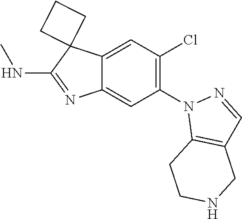 [SMILES: CNC1=Nc2cc(-n3ncc4c3CCNC4)c(Cl)cc2C12CCC2] |
| CA55 | 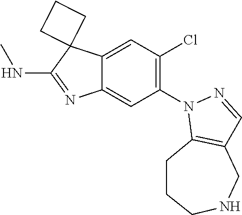 [SMILES: CNC1=Nc2cc(-n3ncc4c3CCCNC4)c(Cl)cc2C12CCC2] |
| CA56 | 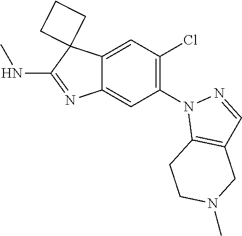 [SMILES: CNC1=Nc2cc(-n3ncc4c3CCN(C)C4)c(Cl)cc2C12CCC2] |
| CA57 | 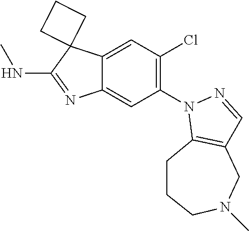 |
| CA58 | 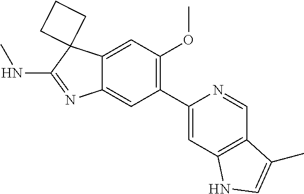 |
| CA59 | 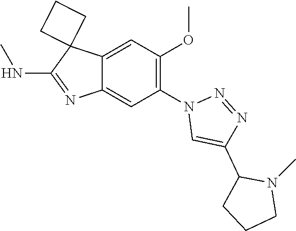 |
| CA59S | 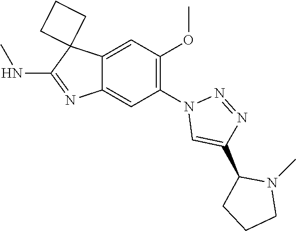 |
| CA59R | 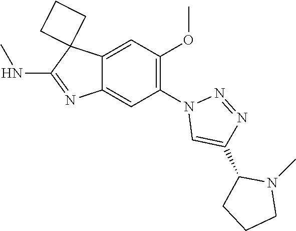 |
| CA60 | 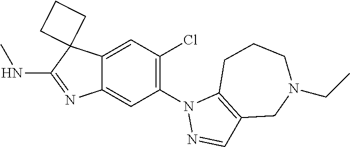 [SMILES: CCN1CCCc2c(cnn2-c2cc3c(cc2Cl)C2(CCC2)C(NC)=N3)C1] |
| CA61 | 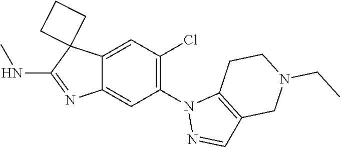 [SMILES: CCN1CCc2c(cnn2-c2cc3c(cc2Cl)C2(CCC2)C(NC)=N3)C1] |
| CA62 | 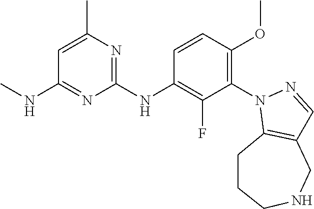 [SMILES: CNc1cc(C)nc(Nc2ccc(OC)c(-n3ncc4c3CCCNC4)c2F)n1] |
| CA63 | 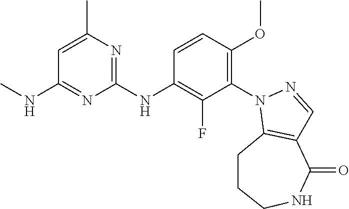 [SMILES: CNc1cc(C)nc(Nc2ccc(OC)c(-n3ncc4c3CCCNC4=O)c2F)n1] |
| CA64 | 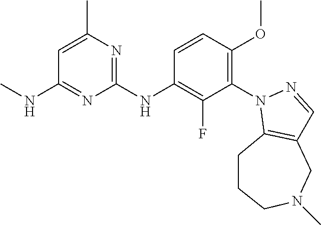 |
| CA65 | 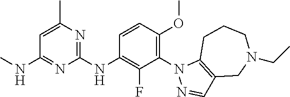 [SMILES: CCN1CCCc2c(cnn2-c2c(OC)ccc(Nc3nc(C)cc(NC)n3)c2F)C1] |
| CA66 | 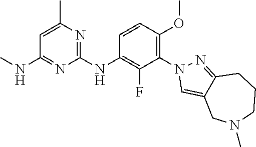 [SMILES: CNc1cc(C)nc(Nc2ccc(OC)c(-n3cc4c(n3)CCCN(C)C4)c2F)n1] |
| CA67 | 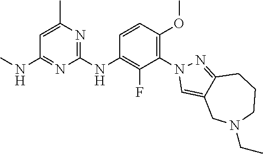 [SMILES: CCN1CCCc2nn(-c3c(OC)ccc(Nc4nc(C)cc(NC)n4)c3F)cc2C1] |
| CA68 | 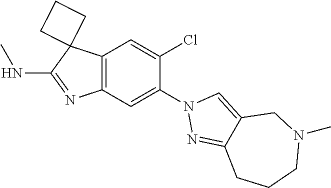 |
| CA69 | 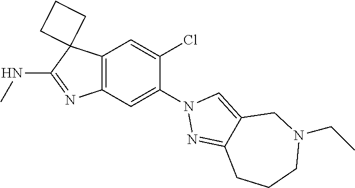 [SMILES: CCN1CCCc2nn(-c3cc4c(cc3Cl)C3(CCC3)C(NC)=N4)cc2C1] |
| CA70 | 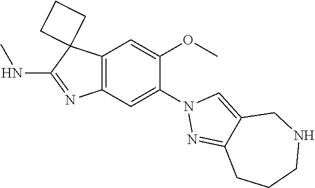 [SMILES: CNC1=Nc2cc(-n3cc4c(n3)CCCNC4)c(OC)cc2C12CCC2] |
| CA71 | 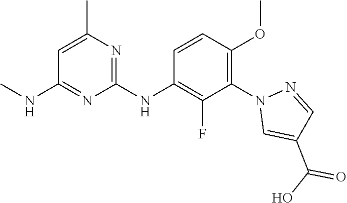 [SMILES: CNc1cc(C)nc(Nc2ccc(OC)c(-n3cc(C(=O)O)cn3)c2F)n1] |
| CA72 | 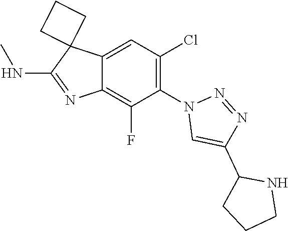 [SMILES: CNC1=Nc2c(cc(Cl)c(-n3cc(C4CCCN4)nn3)c2F)C12CCC2] |
| CA72S | 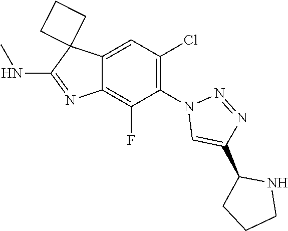 [SMILES: CNC1=Nc2c(cc(Cl)c(-n3cc([C@@H]4CCCN4)nn3)c2F)C12CCC2] |
| CA72R | 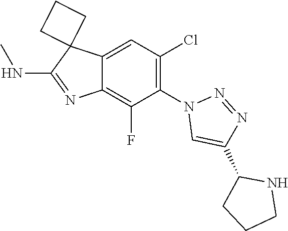 [SMILES: CNC1=Nc2c(cc(Cl)c(-n3cc([C@H]4CCCN4)nn3)c2F)C12CCC2] |
| CA73 | 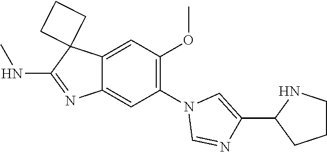 [SMILES: CNC1=Nc2cc(-n3cnc(C4CCCN4)c3)c(OC)cc2C12CCC2] |
| CA73S | 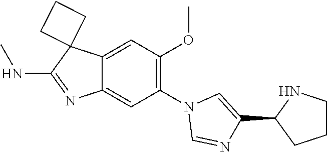 [SMILES: CNC1=Nc2cc(-n3cnc([C@@H]4CCCN4)c3)c(OC)cc2C12CCC2] |
| CA73R | 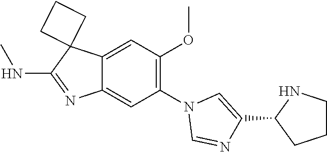 [SMILES: CNC1=Nc2cc(-n3cnc([C@H]4CCCN4)c3)c(OC)cc2C12CCC2] |
| CA74 | 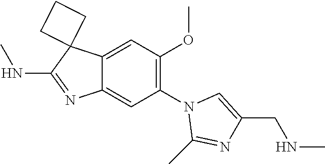 |
| CA75 | 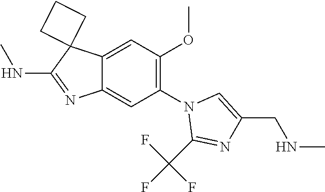 |
| CA76 | 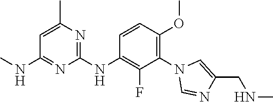 |

The compounds of Table 4A are the compounds found in U.S. Application Nos. 62/681,804, 62/746,252, and 62/746,495, and PCT Application No. PCT/US2018/056333, the entire contents of which are incorporated herein by reference.

TABLE 5

| Compound No. | Structure |
|---|---|
| D1 | 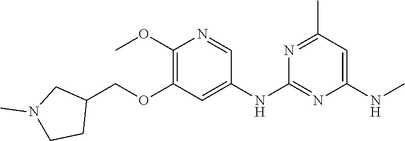 |
| D1R | 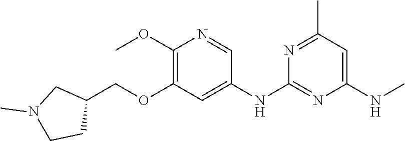 |
| D1S | 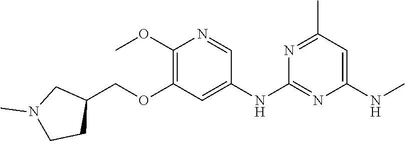 |
| D2 | 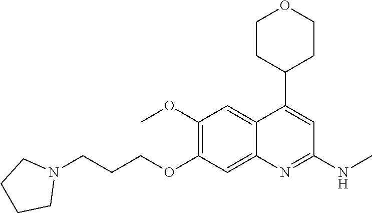 [SMILES: CNc1cc(C2CCOCC2)c2cc(OC)c(OCCCN3CCCC3)cc2n1] |
| D3 | 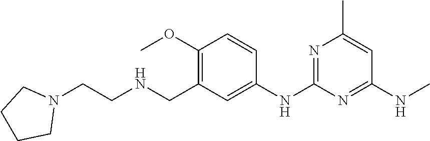 [SMILES: CNc1cc(C)nc(Nc2ccc(OC)c(CNCCN3CCCC3)c2)n1] |
| D4 | 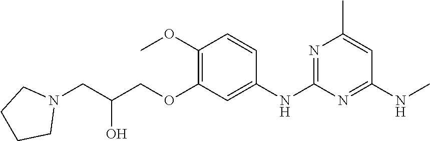 [SMILES: CNc1cc(C)nc(Nc2ccc(OC)c(OCC(O)CN3CCCC3)c2)n1] |
| D4R | 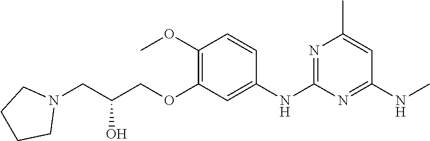 [SMILES: CNc1cc(C)nc(Nc2ccc(OC)c(OC[C@H](O)CN3CCCC3)c2)n1] |
| D4S | 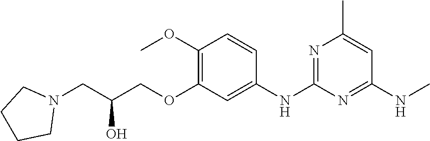 [SMILES: CNc1cc(C)nc(Nc2ccc(OC)c(OC[C@@H](O)CN3CCCC3)c2)n1] |
| D5 | 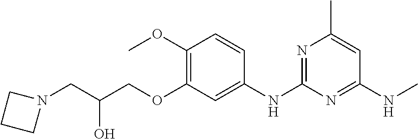 [SMILES: CNc1cc(C)nc(Nc2ccc(OC)c(OCC(O)CN3CCC3)c2)n1] |
| D5R | 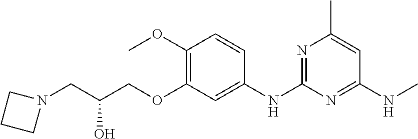 [SMILES: CNc1cc(C)nc(Nc2ccc(OC)c(OC[C@H](O)CN3CCC3)c2)n1] |
| D5S | 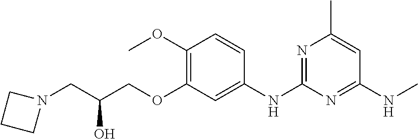 [SMILES: CNc1cc(C)nc(Nc2ccc(OC)c(OC[C@@H](O)CN3CCC3)c2)n1] |
| D6 | 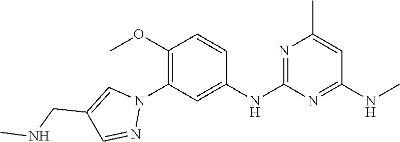 |
| D7 | 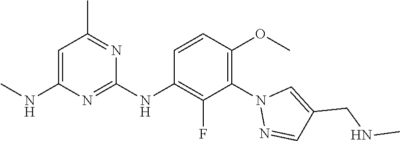 |

The compounds of Table 5 are the compounds found in U.S. Application No. 62/573,917, and PCT Application No. PCT/US2018/056428, the entire contents of which are incorporated herein by reference.

In some embodiments, the EHMT2 inhibitor is a compound selected from Compound Nos. A75, CA51, CA70, DIR, D2, D3, D4R, D5R, D6, and D7, tautomers thereof, pharmaceutically acceptable salts thereof, and pharmaceutically acceptable salts of the tautomers.

In some embodiments, the EHMT2 inhibitor is a compound selected from Compound Nos. A75, CA51, CA70, D1R, D2, D3, D4R, D5R, D6, and D7, and pharmaceutically acceptable salts thereof.

In some embodiments, the EHMT2 inhibitor is a compound selected from Compound Nos. A75, CA51, CA70, D1R, D2, D3, D4R, D5R, D6, and D7.

In some embodiments, the EHMT2 inhibitor is Compound No. A75 or a pharmaceutically acceptable salt thereof.

In some embodiments, the EHMT2 inhibitor is Compound No. A75.

In some embodiments, the EHMT2 inhibitor is Compound No. CA51 or a pharmaceutically acceptable salt thereof.

In some embodiments, the EHMT2 inhibitor is Compound No. CA51.

In some embodiments, the EHMT2 inhibitor is Compound No. CA70 or a pharmaceutically acceptable salt thereof.

In some embodiments, the EHMT2 inhibitor is Compound No. CA70.

In some embodiments, the EHMT2 inhibitor is Compound No. D1R or a pharmaceutically acceptable salt thereof.

In some embodiments, the EHMT2 inhibitor is Compound No. DIR.

In some embodiments, the EHMT2 inhibitor is Compound No. D2 or a pharmaceutically acceptable salt thereof.

In some embodiments, the EHMT2 inhibitor is Compound No. D2

In some embodiments, the EHMT2 inhibitor is Compound No. D3 or a pharmaceutically acceptable salt thereof.

In some embodiments, the EHMT2 inhibitor is Compound No. D3.

In some embodiments, the EHMT2 inhibitor is Compound No. D4R or a pharmaceutically acceptable salt thereof.

In some embodiments, the EHMT2 inhibitor is Compound No. D4R.

In some embodiments, the EHMT2 inhibitor is Compound No. D5R or a pharmaceutically acceptable salt thereof.

In some embodiments, the EHMT2 inhibitor is Compound No. D5R.

In some embodiments, the EHMT2 inhibitor is Compound No. D6 or a pharmaceutically acceptable salt thereof.

In some embodiments, the EHMT2 inhibitor is Compound No. D6.

In some embodiments, the EHMT2 inhibitor is Compound No. D7 or a pharmaceutically acceptable salt thereof.

In some embodiments, the EHMT2 inhibitor is Compound No. D7.

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intended to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, s-pentyl or n-hexyl.

In certain embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “cycloalkyl” refers to a saturated or unsaturated nonaromatic hydrocarbon mono- or multi-ring (e.g., fused, bridged, or spiro rings) system having 3 to 30 carbon atoms (e.g., C₃-C₁₂, C₃-C₁₀, or C₃-C₈). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl.

The term “heterocycloalkyl” refers to a saturated, partially unsaturated, or unsaturated nonaromatic 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, and the like. In the case of multicyclic non-aromatic rings, only one of the rings needs to be non-aromatic (e.g., 1,2,3,4-tetrahydronaphthalenyl or 2,3-dihydroindole).

The term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, “alkyl linker” or “alkylene linker” is intended to include C₁, C₂, C₃, C₄, C₅ or C₆ straight chain (linear) saturated divalent aliphatic hydrocarbon groups and C₃, C₄, C₅ or C₆ branched saturated aliphatic hydrocarbon groups. For example, C₁-C₆ alkylene linker is intended to include C₁, C₂, C₃, C₄, C₅ and C₆ alkylene linker groups. Examples of alkylene linker include, moieties having from one to six carbon atoms, such as, but not limited to, methyl (—CH₂—), ethyl (—CH₂CH₂—), n-propyl (—CH₂CH₂CH₂—), i-propyl (—CHCH₃CH₂—), n-butyl (—CH₂CH₂CH₂CH₂—), s-butyl (—CHCH₃CH₂CH₂—), i-butyl (—C(CH₃)₂CH₂—), n-pentyl (—CH₂CH₂CH₂CH₂CH₂—), s-pentyl (—CHCH₃CH₂CH₂CH₂—) or n-hexyl (—CH₂CH₂CH₂CH₂CH₂CH₂—).

“Alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups.

In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

The term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

“Alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms. As used herein, “C₂-C₆ alkenylene linker” or “C₂-C₆ alkynylene linker” is intended to include C₂, C₃, C₄, C₅ or C₆ chain (linear or branched) divalent unsaturated aliphatic hydrocarbon groups. For example, C₂-C₆ alkenylene linker is intended to include C₂, C₃, C₄, C₅ and C₆ alkenylene linker groups.

The term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

“Aryl” includes groups with aromaticity, including “conjugated,” or multicyclic systems with one or more aromatic rings and do not contain any heteroatom in the ring structure. Examples include phenyl, naphthalenyl, etc.

“Heteroaryl” groups are aryl groups, as defined above, except having from one to four heteroatoms in the ring structure, and may also be referred to as “aryl heterocycles” or “heteroaromatics.” As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted (i.e., N or NR wherein R is H or other substituents, as defined). The nitrogen and sulfur heteroatoms may optionally be oxidized (i.e., N→O and S(O) p, where p=1 or 2). It is to be noted that total number of S and O atoms in the aromatic heterocycle is not more than 1.

Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like.

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthrydine, indole, benzofuran, purine, benzofuran, deazapurine, indolizine.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

As used herein, “carbocycle” or “carbocyclic ring” is intended to include any stable monocyclic, bicyclic or tricyclic ring having the specified number of carbons, any of which may be saturated, unsaturated, or aromatic. Carbocycle includes cycloalkyl and aryl. For example, a C₃-C₁₄ carbocycle is intended to include a monocyclic, bicyclic or tricyclic ring having 3, 4, 5, 6, 7, 8, 9, 10, 11, 12, 13 or 14 carbon atoms. Examples of carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, fluorenyl, phenyl, naphthyl, indanyl, adamantyl and tetrahydronaphthyl. Bridged rings are also included in the definition of carbocycle, including, for example, [3.3.0]bicyclooctane, [4.3.0]bicyclononane, and [4.4.0]bicyclodecane and [2.2.2]bicyclooctane. A bridged ring occurs when one or more carbon atoms link two non-adjacent carbon atoms. In some embodiments, bridge rings are one or two carbon atoms. It is noted that a bridge always converts a monocyclic ring into a tricyclic ring. When a ring is bridged, the substituents recited for the ring may also be present on the bridge. Fused (e.g., naphthyl, tetrahydronaphthyl) and spiro rings are also included.

As used herein, “heterocycle” or “heterocyclic group” includes any ring structure (saturated, unsaturated, or aromatic) which contains at least one ring heteroatom (e.g., 1-4 heteroatoms selected from N, O and S). Heterocycle includes heterocycloalkyl and heteroaryl. Examples of heterocycles include, but are not limited to, morpholine, pyrrolidine, tetrahydrothiophene, piperidine, piperazine, oxetane, pyran, tetrahydropyran, azetidine, and tetrahydrofuran.

Examples of heterocyclic groups include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl (e.g., benzo[d][1,3]dioxole-5-yl), morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, 1,2,4-oxadiazol5(4H)-one, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl and xanthenyl.

The term “substituted,” as used herein, means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N=N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

The term “hydroxy” or “hydroxyl” includes groups with an —OH or —O—.

As used herein, “halo” or “halogen” refers to fluoro, chloro, bromo and iodo. The term “perhalogenated” generally refers to a moiety wherein all hydrogen atoms are replaced by halogen atoms. The term “haloalkyl” or “haloalkoxyl” refers to an alkyl or alkoxyl substituted with one or more halogen atoms.

The term “carbonyl” includes compounds and moieties which contain a carbon connected with a double bond to an oxygen atom. Examples of moieties containing a carbonyl include, but are not limited to, aldehydes, ketones, carboxylic acids, amides, esters, anhydrides, etc.

The term “carboxyl” refers to —COOH or its C₁-C₆ alkyl ester.

“Acyl” includes moieties that contain the acyl radical (R—C(O)—) or a carbonyl group. “Substituted acyl” includes acyl groups where one or more of the hydrogen atoms are replaced by, for example, alkyl groups, alkynyl groups, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Aroyl” includes moieties with an aryl or heteroaromatic moiety bound to a carbonyl group. Examples of aroyl groups include phenylcarboxy, naphthyl carboxy, etc.

“Alkoxyalkyl,” “alkylaminoalkyl,” and “thioalkoxyalkyl” include alkyl groups, as described above, wherein oxygen, nitrogen, or sulfur atoms replace one or more hydrocarbon backbone carbon atoms.

The term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

The term “ether” or “alkoxy” includes compounds or moieties which contain an oxygen bonded to two carbon atoms or heteroatoms. For example, the term includes “alkoxyalkyl,” which refers to an alkyl, alkenyl, or alkynyl group covalently bonded to an oxygen atom which is covalently bonded to an alkyl group.

The term “ester” includes compounds or moieties which contain a carbon or a heteroatom bound to an oxygen atom which is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc.

The term “thioalkyl” includes compounds or moieties which contain an alkyl group connected with a sulfur atom. The thioalkyl groups can be substituted with groups such as alkyl, alkenyl, alkynyl, halogen, hydroxyl, alkylcarbonyloxy, arylcarbonyloxy, alkoxycarbonyloxy, aryloxycarbonyloxy, carboxylate, carboxyacid, alkylcarbonyl, arylcarbonyl, alkoxycarbonyl, aminocarbonyl, alkylaminocarbonyl, dialkylaminocarbonyl, alkylthiocarbonyl, alkoxyl, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties.

The term “thiocarbonyl” or “thiocarboxy” includes compounds and moieties which contain a carbon connected with a double bond to a sulfur atom.

The term “thioether” includes moieties which contain a sulfur atom bonded to two carbon atoms or heteroatoms. Examples of thioethers include, but are not limited to alkthioalkyls, alkthioalkenyls, and alkthioalkynyls. The term “alkthioalkyls” include moieties with an alkyl, alkenyl, or alkynyl group bonded to a sulfur atom which is bonded to an alkyl group. Similarly, the term “alkthioalkenyls” refers to moieties wherein an alkyl, alkenyl or alkynyl group is bonded to a sulfur atom which is covalently bonded to an alkenyl group; and “alkthioalkynyls” refers to moieties wherein an alkyl, alkenyl or alkynyl group is bonded to a sulfur atom which is covalently bonded to an alkynyl group.

As used herein, “amine” or “amino” refers to —NH₂. “Alkylamino” includes groups of compounds wherein the nitrogen of —NH₂ is bound to at least one alkyl group. Examples of alkylamino groups include benzylamino, methylamino, ethylamino, phenethylamino, etc. “Dialkylamino” includes groups wherein the nitrogen of —NH₂ is bound to two alkyl groups. Examples of dialkylamino groups include, but are not limited to, dimethylamino and diethylamino. “Arylamino” and “diarylamino” include groups wherein the nitrogen is bound to at least one or two aryl groups, respectively. “Aminoaryl” and “aminoaryloxy” refer to aryl and aryloxy substituted with amino. “Alkylarylamino,” “alkylaminoaryl” or “arylaminoalkyl” refers to an amino group which is bound to at least one alkyl group and at least one aryl group. “Alkaminoalkyl” refers to an alkyl, alkenyl, or alkynyl group bound to a nitrogen atom which is also bound to an alkyl group. “Acylamino” includes groups wherein nitrogen is bound to an acyl group. Examples of acylamino include, but are not limited to, alkylcarbonylamino, arylcarbonylamino, carbamoyl and ureido groups.

The term “amide” or “aminocarboxy” includes compounds or moieties that contain a nitrogen atom that is bound to the carbon of a carbonyl or a thiocarbonyl group. The term includes “alkaminocarboxy” groups that include alkyl, alkenyl or alkynyl groups bound to an amino group which is bound to the carbon of a carbonyl or thiocarbonyl group. It also includes “arylaminocarboxy” groups that include aryl or heteroaryl moieties bound to an amino group that is bound to the carbon of a carbonyl or thiocarbonyl group. The terms “alkylaminocarboxy”, “alkenylaminocarboxy”, “alkynylaminocarboxy” and “arylaminocarboxy” include moieties wherein alkyl, alkenyl, alkynyl and aryl moieties, respectively, are bound to a nitrogen atom which is in turn bound to the carbon of a carbonyl group. Amides can be substituted with substituents such as straight chain alkyl, branched alkyl, cycloalkyl, aryl, heteroaryl or heterocycle. Substituents on amide groups may be further substituted.

Compounds of the present disclosure that contain nitrogens can be converted to N-oxides by treatment with an oxidizing agent (e.g., 3-chloroperoxybenzoic acid (mCPBA) and/or hydrogen peroxides) to afford other compounds of the present disclosure. Thus, all shown and claimed nitrogen-containing compounds are considered, when allowed by valency and structure, to include both the compound as shown and its N-oxide derivative (which can be designated as N→O or N⁺—O⁻). Furthermore, in other instances, the nitrogens in the compounds of the present disclosure can be converted to N-hydroxy or N-alkoxy compounds. For example, N-hydroxy compounds can be prepared by oxidation of the parent amine by an oxidizing agent such as m-CPBA. All shown and claimed nitrogen-containing compounds are also considered, when allowed by valency and structure, to cover both the compound as shown and its N-hydroxy (i.e., N—OH) and N-alkoxy (i.e., N—OR, wherein R is substituted or unsubstituted C₁-C₆ alkyl, C₁-C₆ alkenyl, C₁-C₆ alkynyl, 3-14-membered carbocycle or 3-14-membered heterocycle) derivatives.

In the present specification, the structural formula of the compound represents a certain isomer for convenience in some cases, but the present disclosure includes all isomers, such as geometrical isomers, optical isomers based on an asymmetrical carbon, stereoisomers, tautomers, and the like, it being understood that not all isomers may have the same level of activity. In addition, a crystal polymorphism may be present for the compounds represented by the formula. It is noted that any crystal form, crystal form mixture, or anhydride or hydrate thereof is included in the scope of the present disclosure.

“Isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

A carbon atom bonded to four nonidentical substituents is termed a “chiral center.”

“Chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Eit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

“Geometric isomer” means the diastereomers that owe their existence to hindered rotation about double bonds or a cycloalkyl linker (e.g., 1,3-cylcobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It should also be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

Furthermore, the structures and other compounds discussed in this disclosure include all atropic isomers thereof, it being understood that not all atropic isomers may have the same level of activity. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertible by tautomerizations is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), imine-enamine and enamine-enamine. Examples of lactam-lactim tautomerism are as shown below.

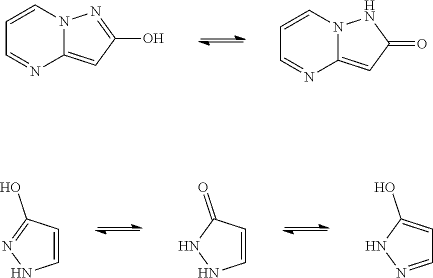 [SMILES: Oc1cc2ncccn2n1 ; O=c1cc2ncccn2[nH]1 ; Oc1cc[nH]n1 ; O=c1cc[nH][nH]1 ; Oc1ccn[nH]1]

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

The term “crystal polymorphs”, “polymorphs” or “crystal forms” means crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

The compounds of any Formula described herein include the compounds themselves, as well as their salts, and their solvates, if applicable. A salt, for example, can be formed between an anion and a positively charged group (e.g., amino) on a substituted benzene compound. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate). The term “pharmaceutically acceptable anion” refers to an anion suitable for forming a pharmaceutically acceptable salt. Likewise, a salt can also be formed between a cation and a negatively charged group (e.g., carboxylate) on a substituted benzene compound. Suitable cations include sodium ion, potassium ion, magnesium ion, calcium ion, and an ammonium cation such as tetramethylammonium ion. The substituted benzene compounds also include those salts containing quaternary nitrogen atoms.

Additionally, the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

“Solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As defined herein, the term “derivative” refers to compounds that have a common core structure, and are substituted with various groups as described herein. For example, all of the compounds represented by Formula (II) are substituted bi-heterocyclic compounds, and have Formula (II) as a common core.

The term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonimides, tetrazoles, sulfonates and phosphonates. See, e.g., Patani and LaVoie, Chem. Rev. 96, 3147-3176, 1996.

The present disclosure is intended to include all isotopes of atoms occurring in the present compounds. Isotopes include those atoms having the same atomic number but different mass numbers. By way of general example and without limitation, isotopes of hydrogen include tritium and deuterium, and isotopes of carbon include C-13 and C-14.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

The present disclosure provides methods for the synthesis of the compounds of any of the Formulae described herein. The present disclosure also provides detailed methods for the synthesis of various disclosed compounds of the present disclosure according to the following schemes as well as those shown in the Examples.

Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the respective embodiments remain operable. Moreover, two or more steps or actions can be conducted simultaneously.

The synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

Compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Afechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagentsfor Organic Synthesis, John Wiley and Sons (1995), incorporated by reference herein, are useful and recognized reference textbooks of organic synthesis known to those in the art. The following descriptions of synthetic methods are designed to illustrate, but not to limit, general procedures for the preparation of compounds of the present disclosure.

Compounds of the present disclosure can be conveniently prepared by a variety of methods familiar to those skilled in the art.

One of ordinary skill in the art will note that, during the reaction sequences and synthetic schemes described herein, the order of certain steps may be changed, such as the introduction and removal of protecting groups.

One of ordinary skill in the art will recognize that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

Some aspects of this disclosure provide that compounds that inhibit the histone methyltransferase activity of G9a, also known as KMTIC (lysine methyltransferase 1C) or EHMT2 (euchromatic histone methyltransferase 2), or a mutant thereof are useful for treating and/or preventing certain conditions, diseases, and disorders in which EHMT2 plays a role, e.g., certain blood disorders disclosed herein. The present disclosure provides methods for treating conditions, diseases, and disorders, the course of which can be influenced by modulating the methylation status of histones or other proteins, wherein said methylation status is mediated at least in part by the activity of EHMT2. Modulation of the methylation status of histones can in turn influence the level of expression of target genes activated by methylation, and/or target genes suppressed by methylation. The therapeutic methods provided herein typically comprise administering to a subject in need of such treatment, a therapeutically effective amount of an EHMT2 inhibitor, e.g., of an EHMT2 inhibitory compound provided herein, or a pharmaceutically acceptable salt, polymorph, solvate, or stereoisomer thereof.

Unless otherwise stated, any description of a method of treatment includes use of the respective agent(s), e.g., an EHMT2 inhibitor, to provide such treatment or prophylaxis as is described herein, as well as use of such an agent, e.g., of the EHMT2 inhibitor, to prepare a medicament to treat or prevent such condition.

In still another aspect, this disclosure relates to a method of modulating the activity of EHMT2, which catalyzes the dimethylation of lysine 9 on histone H3 (H3K9) in a subject in need thereof.

The present disclosure also provides methods for treating conditions and diseases the course of which can be influenced by modulating the methylation status of histones or other proteins, wherein said methylation status is mediated at least in part by the activity of EHMT2, by administering to a subject having such a disease or condition, or being at risk of developing such a disease or condition, an EHMT2 inhibitor, e.g., an EHMT2 inhibitor provided herein. Modulation of the methylation status of histones can in turn influence the level of expression of target genes activated by methylation, and/or target genes suppressed by methylation.

For example, certain methods and compounds disclosed herein are useful for preventing or treating a blood disorder (e.g., sickle-cell disease).

As used herein, a “subject” is interchangeable with a “subject in need thereof”, both of which refer to a subject having a disorder in which EHMT2-mediated protein methylation plays a part, or a subject having an increased risk of developing such disorder relative to the population at large. A “subject” includes a mammal. The mammal can be e.g., a human or appropriate non-human mammal, such as primate, rodent, mouse, rat, dog, cat, cow, horse, goat, camel, sheep or a pig. The subject can also be a bird or fowl. In some embodiments, the subject is a human. A subject in need thereof can be one who has been previously diagnosed or identified as having a blood disorder. A subject in need thereof can also be one who has (e.g., is suffering from) a blood disorder. In some embodiments, a subject in need thereof can be one who has an increased risk of developing such disorder relative to the population at large (e.g., a subject who is predisposed to developing such disorder relative to the population at large). A subject in need thereof can have a refractory or resistant blood disorder (e.g., a blood disorder that doesn't respond or hasn't yet responded to treatment). The subject may be resistant at start of treatment or may become resistant during treatment. In some embodiments, the subject in need thereof received and failed all known effective therapies for a blood disorder. In some embodiments, the subject in need thereof received at least one prior therapy. In a preferred embodiment, the subject has a blood disorder. In some embodiments, the blood disorder is Acute lymphoblastic leukemia (ALL), Acute myeloid leukemia (AML) (e.g., acute promyelocytic leukemia, APL), Amyloidosis, Anemia, Aplastic anemia, Bone marrow failure syndromes, Chronic lymphocytic leukemia (CLL), Chronic myeloid leukemia (CML), Deep vein thrombosis (DVT), Diamond-Blackfan anemia, Dyskeratosis congenita (DKC), Eosinophilic disorder, Essential thrombocythemia, Fanconi anemia, Gaucher disease, Hemochromatosis, Hemolytic anemia, Hemophilia, Hereditary spherocytosis, Hodgkin's lymphoma, Idiopathic thrombocytopenic purpura (ITP), Inherited bone marrow failure syndromes, Iron-deficiency anemia, Langerhans cell histiocytosis, Large granular lymphocytic (LGL) leukemia, Leukemia, Leukopenia, Mastocytosis, Monoclonal gammopathy, Multiple myeloma, Myelodysplastic syndromes (MDS), Myelofibrosis, Myeloproliferative neoplasms (MPN), Non-Hodgkin's lymphoma, Paroxysmal nocturnal hemoglobinuria (PNH), Pernicious anemia (B12 deficiency), Polycythemia vera, Porphyria, Post-transplant lymphoproliferative disorder (PTLD), Pulmonary embolism (PE), Shwachman-Diamond syndrome (SDS), Sickle-cell disease (SCD), Thalassemias, Thrombocytopenia, Thrombotic thrombocytopenic purpura (TTP), Venous thromboembolism, Von Willebrand disease, or Waldenstrom's macroglobulinemia (lymphoplasmacytic lymphoma). In some embodiments, the subject has sickle-cell disease. In some embodiments, the subject has a blood disorder known to those skilled in the art, e.g., a blood disorder described in Table 6 below, and in Kim et al., Nature Medicine 23:213-222, 2017 and Soellner et al., Clinical Genetics 91:3-13, 2017

TABLE 6

| | | Molecular | Frequencies | | | |
|---|---|---|---|---|---|---|
| Disorder | Chromosome | Alterations | (%) | MLID | recurrence risk | Clinical features |
| Transient Neonatal | 6q24 | UPD(6)pat | 41 | | <1% | IUGR, transient |
| Diabetes mellitus | | | | | | diabetes, |
| (TNDM) | | | | | | hyperglycemia |
| | | | | | | without ketoacidosis, |
| | | | | | | macroglossia, |
| | | | | | | omphalocele |
| | | dup(6q) | 29 | | Increased in case of | |
| | | | | | a paternal structural | |
| | | | | | variation | |
| | | PLAGL1:alt-TSS- | 30% | 50% | In case of a ZFP57 | |
| | | DMR: LOM | | | mutation | |
| Silver-Russell | 7 | upd(7)mat | 7-10% | 1 case | <1%, but a single | IUGR/PNGR, relative |
| syndrome (SRS) | | | | | familial structural | macrocephaly, |
| | | | | | variation has been | asymmetry, G1 |
| | | | | | reported | prominent |
| | | | | | | forehead/triangular |
| | | | | | | face, feeding |
| | | | | | | difficulties |
| | | CNVs (dup7p), | Single cases | | Increased in case of | |
| | | del7q | | | a familial structural | |
| | | | | | variation | |
| | 11p15.5 | upd(11)mat | n = 1 | — | <1% | |
| | | upd(11p15)mat | 1-2% | — | Increased in case of | |
| | | | | | a familial structural | |
| | | | | | variation | |
| | | H19/IGF2:IG- | >38% | ~10% | Only single families, | |
| | | DMR: LOM | | | risk might be | |
| | | | | | increased in case of | |
| | | | | | MLID | |
| | | CDKN1C | n = 1 | — | 50% in case of | |
| | | mutations | | | maternal | |
| | | | | | transmission | |
| | | IGF2 mutations | n = 1 | — | 50% in case of | |
| | | | | | paternal | |
| | | | | | transmission | |
| Birk-Barel mental | 8q24.3 | KCNK9 mutations | Unknown | — | 50% in case of | Intellectual disability, |
| retardation | | | | | maternal | hyperactivity, feeding |
| | | | | | transmission | difficulties, hypotonia, |
| | | | | | | elongated face |
| Beckwith- | 11p15.5 | upd(11)pat | 20% | — | No | Pre- and postnatal |
| Wiedemann | | | | | | overgrowth, |
| syndrome (BWS) | | | | | | organomegaly, |
| | | | | | | macroglossia, |
| | | | | | | omphalocele, neonatal |
| | | | | | | hypoglycemia, |
| | | | | | | hemihypertrophy, |
| | | | | | | increased tumor risk |
| | | Uniparental | ~10% | | | |
| | | diploidy* | | | | |
| | | Paternal UPD | ~90% | | | |
| | | dup(11p15)pat | 1-2% | — | Increased in case of | |
| | | | | | a familial structural | |
| | | | | | variation | |
| | | H19/IGF2:IG- | 4% | — | 20% (in case of | |
| | | DMR: GOM | | | microdeletions or | |
| | | | | | SNPs in the | |
| | | | | | OCT4/SOX2 | |
| | | | | | binding site) | |
| | | KCNQ1OT1:TSS- | 50% | 25 | Only single families | |
| | | DMR: LOM | | | have been reported, | |
| | | | | | but the risk might be | |
| | | | | | increased shen | |
| | | | | | MLID | |
| | | CDKN1C | 5% | — | 50% in case of | |
| | | mutations | | | maternal | |
| | | | | | transmission | |
| Temple syndrome | 14q32 | upd(14)mat | 78.4% | — | <1%, but increased | IUGR, PNGR, |
| (UPD(14)mat) | | | | | in case of familial | hypotonia, feeding |
| | | | | | Robertsonian | difficulties in infancy, |
| | | | | | translocation | truncal obesity, |
| | | | | | | scoliosis, precocious |
| | | | | | | puberty |
| | | del(14q32)pat | 9.8% | — | <1%, but increased | |
| | | | | | in case of familial | |
| | | | | | translocation | |
| | | MEG3/DLK1:IG- | 11.7% | NR | Unknown | |
| | | DMR | | | | |
| | | and | | | | |
| | | MEG3:TSS-DMR: | | | | |
| | | LOM | | | | |
| Kagami-Ogata | 14q32 | upd(14)pat | 65.4% | — | <1%, but increased | IUGR, |
| syndrome | | | | | in case of familial | polyhydramnion, |
| (UPD(14)pat) | | | | | Robertsonian | abdominal and |
| | | | | | translocation | thoracal wall defects, |
| | | | | | | bell-shaped thorax, |
| | | | | | | coat-hanger ribs |
| | | del(14q32)mat | 19.2% | — | <1%, but increased | |
| | | | | | in case of familial | |
| | | | | | translocation | |
| | | MEG3/DLK1:IG- | 15.4% | NR | | |
| | | DMR | | | | |
| | | and | | | | |
| | | MEG3:TSS-DMR: | | | | |
| | | GOM | | | | |
| Angelman | 15q11q13 | upd(15)pat | 1-2% | — | <1% | Mental retardation, |
| syndrome (AS) | | | | | | microcephaly, no |
| | | | | | | speech, unmotivated |
| | | | | | | laughing, ataxia, |
| | | | | | | seizures |
| | | del(15q11q13)mat | 75% | — | <1%, but increased | |
| | | | | | in case of familial | |
| | | | | | translocation | |
| | | SNURF:TSS- | ~3% | — | Up to 50% | |
| | | DMR: LOM | | | | |
| | | UBE3A mutations | 5-10% | — | Up to 50% | |
| Prader-Willi | 15q11q13 | upd(15)mat | 25-30% | — | <1% | PNGR, mental |
| syndrome (PWS) | | | | | | retardation, neonatal |
| | | | | | | hypotonia, |
| | | | | | | hypogenitalism, |
| | | | | | | hypopigmentation, |
| | | | | | | obesity/hyperphagia |
| | | del(15q11q13)pat | 70-75% | — | <1%, but increased | |
| | | | | | in case of familial | |
| | | | | | translocation | |
| | | SNURF:TSS- | ~1% | 1 case | Up to 50% | |
| | | DMR: GOM | | | | |
| Precocious puberty | 15q11.2 | MKRN3 mutations | Unknown | | 50% in case of | Precocious puberty |
| | | | | | paternal | (girls: 5.75 years, |
| | | | | | transmission | boys: 8.10 years) |
| Schaaf-Yang | 15q11.2 | MAGEL2 | Unknown | — | 50% in case of | Neonatal hypotonia, |
| syndrome | | mutations | | | paternal | feeding problems in |
| (SHFYNG) | | | | | transmission | infancy, then |
| | | | | | | hyperphagia, |
| | | | | | | developmental delay, |
| | | | | | | hypogonadism |
| Sporadic | 20q13 | upd(20)pat | 10-25% | | <1% | Resistance to PTH |
| pseudohypopara- | | | | | | and other hormones, |
| thyreoidism 1b | | | | | | Albright hereditary |
| | | | | | | osteodystrophy, |
| | | | | | | subcutaneous |
| | | | | | | ossifications, feeding |
| | | | | | | behavior anomalies, |
| | | | | | | abnormal growth |
| | | del(20q13) | Rare | | <1%, but increased | |
| | | | | | in case of familial | |
| | | | | | translocation | |
| | | GNAS-NESP:TSS- | >60% | 12.5% | <1% | |
| | | DMR: LOM | | | | |
| | | GNAS-XL:Ex1- | | | | |
| | | DMR:LOM GNAS | | | | |
| | | A/B:TSS-DMR | | | | |
| upd(20)mat | 20 | upd(20)mat | Unknown | 9 cases | <1%, but familial | IUGR, PNGR, failure |
| | | | | | translocation should | to thrive |
| | | | | | be considered | |

Some aspects of the present disclosure provide diagnostic and/or prognostic methods that are useful for predicting the response of a subject having a blood disorder to treatment with an EHMT2 inhibitor. For example, in some embodiments, a method is provided that comprises determining a levels of a globin, e.g., gamma globin and/or fetal hemoglobin (HbF), in a subject having a blood disorder, e.g., sickle-cell disease or a blood disorder described herein, and comparing the level of the globin determined in the subject with a reference or control level, wherein if the level of the globin determined in the subject is higher than the reference or control level, the subject is identified as likely to respond to treatment according to a method provided herein, e.g., to a method comprising administering a therapeutically effective amount of an EHMT2 inhibitor, either alone or in combination with one or more additional therapeutic agent, to the subject. In some embodiments, the control or reference level is a level expected or observed in a healthy subject, e.g., an age- and sex-matched subject. In some embodiments, the reference or control level is an average level measured across a population of subject, e.g., a population of age- and sex-matched subjects. In some embodiments, the control or reference level is an everage level observed amongst subjects having the same disease, e.g., amongst subjects having sickle-cell disease. In some embodiments, the method comprises determining a level of globin mRNA, e.g., a level of gamma-globin mRNA. In some embodiments, the method comprises determining a level of globin protein, e.g., a level of fetal hemoglobin protein (HbF). Suitable methods for determining globin levels in a subject are known to those of ordinary skill in the art, and the present disclosure is not limited in this respect. In some embodiments, the method further comprises administering an EHMT2 inhibitor according to the methods provided herein to the subject identified as likely to respond to such treatment.

In some embodiments, a subject is identified as likely to respond to treatment according to the methods provided herein, if the HbF level before treatment is greater than 0.5% of total hemoglobin. In some embodiments, a subject is identified as likely to respond to treatment according to the methods provided herein, if the HbF level before treatment is greater than 0.75%, greater than 0.8%, greater than 0.9%, greater than 1%, greater than 1.5%, greater than 2%, greater than 2.5%, greater than 3%, greater than 4%, greater than 5%, greater than 7.5%, greater than 10%, greater than 15%, greater than 20%, greater than 25%, or greater than 30% of total hemoglobin.

As used herein, “candidate compound” refers to a compound of the present disclosure, or a pharmaceutically acceptable salt, polymorph or solvate thereof, that has been or will be tested in one or more in vitro or in vivo biological assays, in order to determine if that compound is likely to elicit a desired biological or medical response in a cell, tissue, system, animal or human that is being sought by a researcher or clinician. A candidate compound is a compound of the present disclosure, or a pharmaceutically acceptable salt, polymorph or solvate thereof. The biological or medical response can be treatment or prevention of a blood disorder. The biological response or effect can also include a change in cell proliferation or growth that occurs in vitro or in an animal model, as well as other biological changes that are observable in vitro. In vitro or in vivo biological assays can include, but are not limited to, enzymatic activity assays, electrophoretic mobility shift assays, reporter gene assays, in vitro cell viability assays, and the assays described herein.

In some embodiments, an in vitro biological assay that can be used includes the steps of (1) mixing a histone substrate (e.g., an isolated histone sample or an isolated histone peptide representative of human histone H3 residues 1-15) with recombinant EHMT2 enzymes; (2) adding a compound of the disclosure to this mixture; (3) adding non-radioactive and ³H-labeled S-Adenosyl methionine (SAM) to start the reaction; (4) adding excessive amount of non-radioactive SAM to stop the reaction; (4) washing off the free non-incorporated ³H-SAM; and (5) detecting the quantity of ³H-labeled histone substrate by any methods known in the art (e.g., by a PerkinElmer TopCount platereader).

In some embodiments, an in vitro study that can be used includes the steps of (1) treating blood disorder model cells with a compound of this disclosure; (2) incubating the cells for a set period of time; (3) fixing the cells; (4) treating the cells with primary antibodies that bind to dimethylated histone substrates; (5) treating the cells with a secondary antibody (e.g. an antibody conjugated to an infrared dye); (6) detecting the quantity of bound antibody by any methods known in the art (e.g., by a Licor Odyssey Infrared Scanner).

As used herein, “treating” or “treat” describes the management and care of a patient for the purpose of combating a disease, condition, or disorder and includes the administration of a therapeutic agent, e.g., an EHMT2 inhibitor, e.g., an EHMT2 inhibitor provided herein, or a pharmaceutically acceptable salt, polymorph or solvate thereof, either alone or together with one or more additional therapeutic agent, to alleviate the symptoms or complications of a disease, condition or disorder, or to eliminate the disease, condition or disorder. The term “treat” can also include treatment of a cell in vitro or an animal model.

A compound of the present disclosure, or a pharmaceutically acceptable salt, polymorph or solvate thereof, can or may also be used to prevent a relevant disease, condition or disorder, or used to identify suitable candidates for such purposes. As used herein, “preventing,” “prevent,” or “protecting against” describes reducing or eliminating the onset of the symptoms or complications of such disease, condition or disorder.

One skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18^{th} edition (1990). These texts can, of course, also be referred to in making or using an aspect of the disclosure.

As used herein, “combination therapy” or “co-therapy” includes the administration of a compound of the present disclosure, or a pharmaceutically acceptable salt, polymorph or solvate thereof, and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents.

The present disclosure also provides pharmaceutical compositions comprising a compound of any of the Formulae described herein in combination with at least one pharmaceutically acceptable excipient or carrier.

A “pharmaceutical composition” is a formulation containing the compounds of the present disclosure in a form suitable for administration to a subject. In some embodiments, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In some embodiments, the active compound is mixed under sterile conditions with a pharmaceutically acceptable carrier, and with any preservatives, buffers, or propellants that are required.

As used herein, the phrase “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical composition of the disclosure is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition (e.g., blood disorders, and the like) and the health of the patient should preferably be closely monitored during and for a reasonable period after treatment.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In a preferred aspect, the disease or condition to be treated is a blood disorder. In some embodiments, e.g., in some embodiments disclosed herein that comprise administering an EHMT2 inhibitor to a subject having a blood disorder, e.g., sickle-cell disease (also referred to as sickle-cell anemia), a therapeutically effective amount of an EHMT2 inhibitor is an amount sufficient to raise the fetal hemoglobin (HbF) level in the subject by at least 2-fold, at least 3-fold, at least 4-fold, at least 5-fold, at least 10-fold, at least 15-fold, at least 10-fold, at least 30-fold, at least 50-fold, at least 100-fold, or at least 1000-fold. In some embodiments, e.g., in some embodiments disclosed herein that comprise administering an EHMT2 inhibitor to a subject having a blood disorder, e.g., sickle-cell disease (also referred to as sickle-cell anemia), a therapeutically effective amount of an EHMT2 inhibitor is an amount sufficient to raise the fetal hemoglobin (HbF) level in the subject to at least 1%, at least 2%, at least 3%, at least 4%, at least 5%, at least 6%, at least 7%, at least 8%, at least 9%, at least 10%, at least 15%, at least 20%/0, at least 25%, at least 30%, at least 40%, or at least 50% of total hemoglobin in the subject.

For any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose used in the methods provided herein should be sufficient to result in slowing, and preferably regressing, the symptoms of the blood disorder and also preferably causing complete regression of the blood disorder. Dosages can range from about 0.01 mg/kg per day to about 5000 mg/kg per day. In preferred aspects, dosages can range from about 1 mg/kg per day to about 1000 mg/kg per day. In an aspect, the dose will be in the range of about 0.1 mg/day to about 50 g/day; about 0.1 mg/day to about 25 g/day; about 0.1 mg/day to about 10 g/day; about 0.1 mg to about 3 g/day; or about 0.1 mg to about 1 g/day, in single, divided, or continuous doses (which dose may be adjusted for the patient's weight in kg, body surface area in m², and age in years). An effective amount of a pharmaceutical agent is that which provides an objectively identifiable improvement as noted by the clinician or other qualified observer. Improvement in survival and growth indicates regression. As used herein, the term “dosage effective manner” refers to amount of an active compound to produce the desired biological effect in a subject or cell.

The pharmaceutical compositions can be included in a container, pack, or dispenser together with instructions for administration.

The compounds of the present disclosure are capable of further forming salts. All of these forms are also contemplated within the scope of the claimed disclosure.

As used herein, “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ration other than 1:1, e.g., 3:1, 2:1, 1:2, or 1:3.

It should be understood that all references to pharmaceutically acceptable salts include solvent addition forms (solvates) or crystal forms (polymorphs) as defined herein, of the same salt.

The compounds of the present disclosure can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., acetate, propionate or other ester.

The compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In some embodiments, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the disclosure can be found in Remington: the Science and Practice of Pharmacy, 19^{th} edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. Some non-limiting embodiments having now been described by way of written description, those of skill in the art will recognize that the concepts, strategies, methods, and aspects of this disclosure can be practiced in a variety of embodiments and that the foregoing description and examples below are for purposes of illustration and not limitation of the claims that follow.

### Example 1: Synthesis of EHMT2 Inhibitor Compounds

EHMT2 inhibitor compounds useful for the treatment of blood disorders as provided herein were synthesized or may be synthesized by, e.g., methods described in U.S. Application Nos. 62/323,602, 62/348,837, 62/402,997, 62/402,863, 62/509,620, 62/436,139, 62/517,840, 62/573,442, 62/681,804, 62/746,252, and 62/746,495, and Ser. No. 15/601,888, and PCT Application Nos. PCT/US2017/027918, PCT/US2017/054468, PCT/US2017/067192, PCT/US2018/056333, and PCT/US2018/056428, the contents of each of which are incorporated herein by reference in their entireties.

### Example 2: Treatment of Sickle-Cell Anemia

A first subject having sickle-cell disease is administered an EHMT2 inhibitor provided herein. The EHMT2 inhibitor is administered to the subject at a dose sufficient to inhibit more than 90% EHMT2 methyltransferase activity in the subject and/or to increase fetal hemoglobin (HbF) levels to at least 20% total hemoglobin in the subject.

A second subject is treated with a similar EHMT2 inhibitor regimen as the first subject, and also administered hydroxyurea at a dose used for the clinical treatment of sickle-cell anemia.

A third subject is treated with a similar EHMT2 inhibitor regimen as the first subject, and also administered L-glutamine at a dose used for the clinical treatment of sickle-cell anemia.

A fourth subject is treated with a similar EHMT2 inhibitor regimen as the first subject, and also administered hydroxyurea and L-Glutamine at a dose used for the clinical treatment of sickle-cell anemia.

### Example 3: In Vitro Combination Studies of EHMT2 Inhibitor Compounds with Other Agents

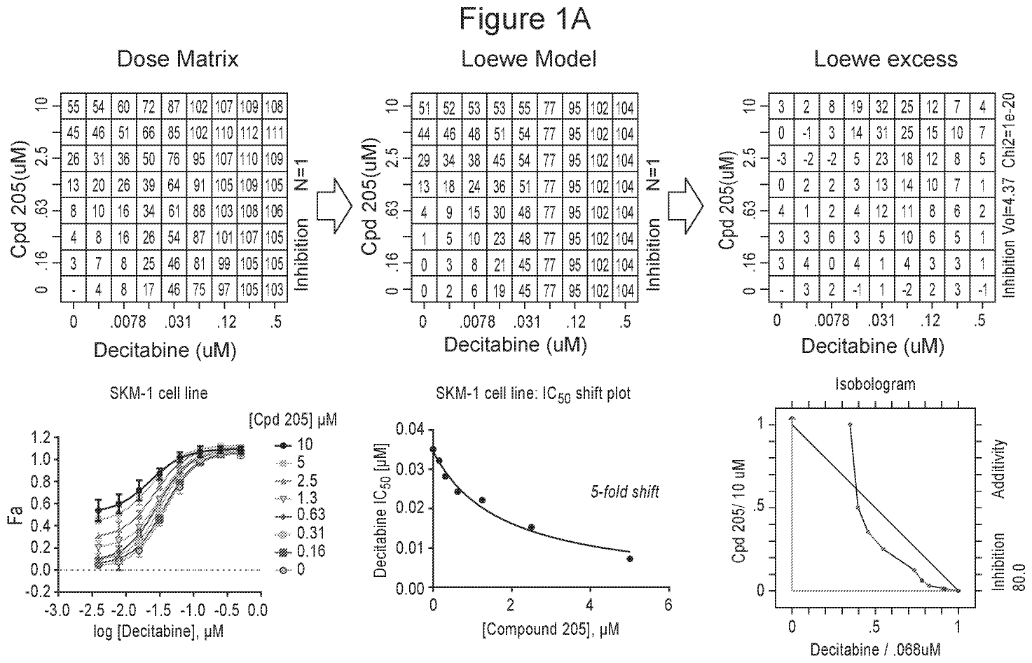

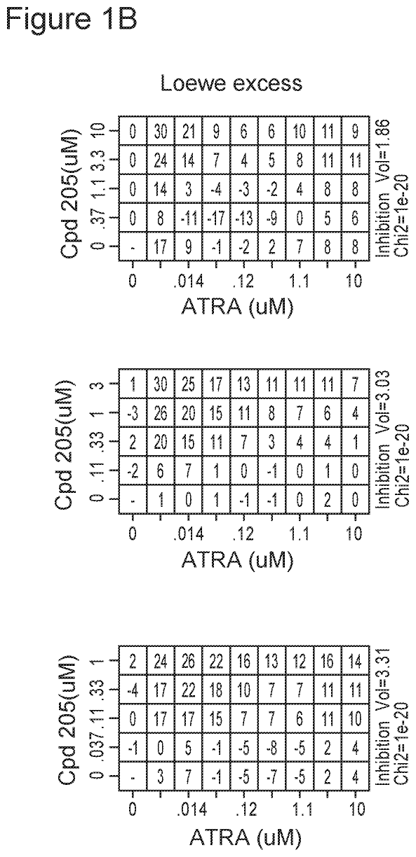

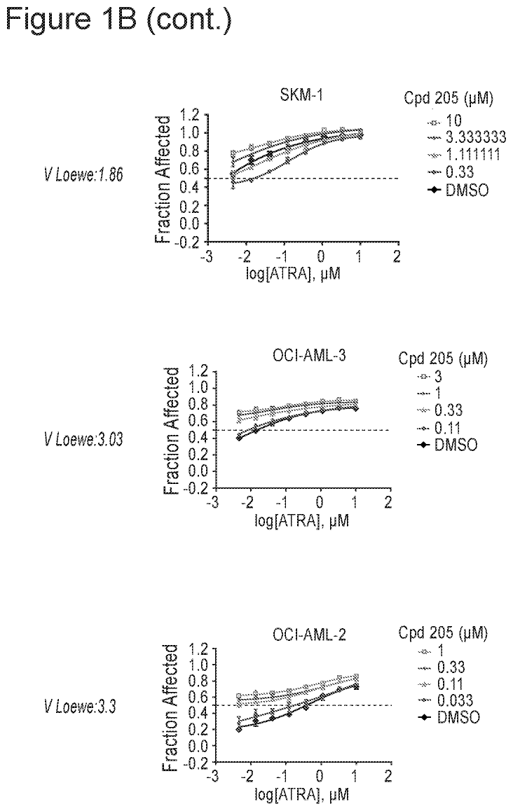

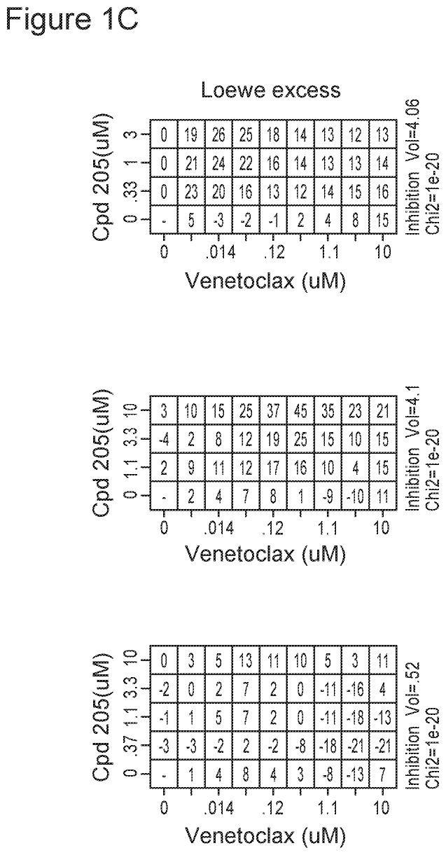

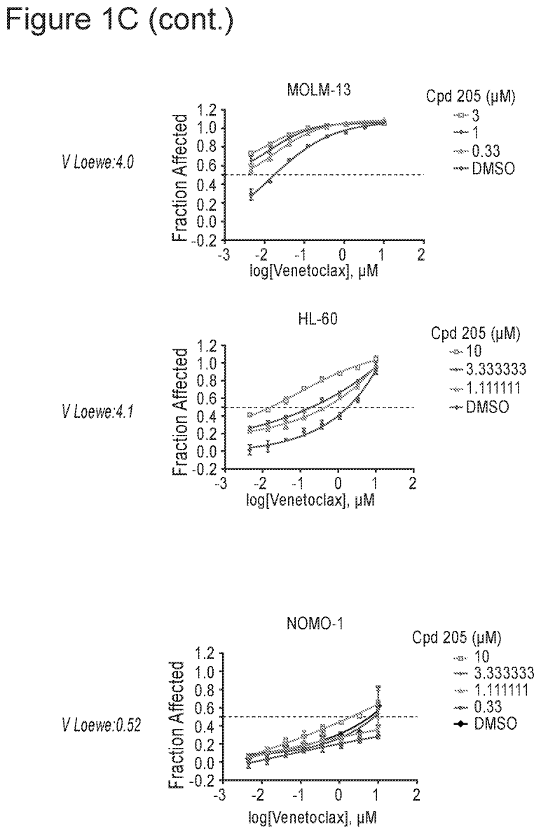

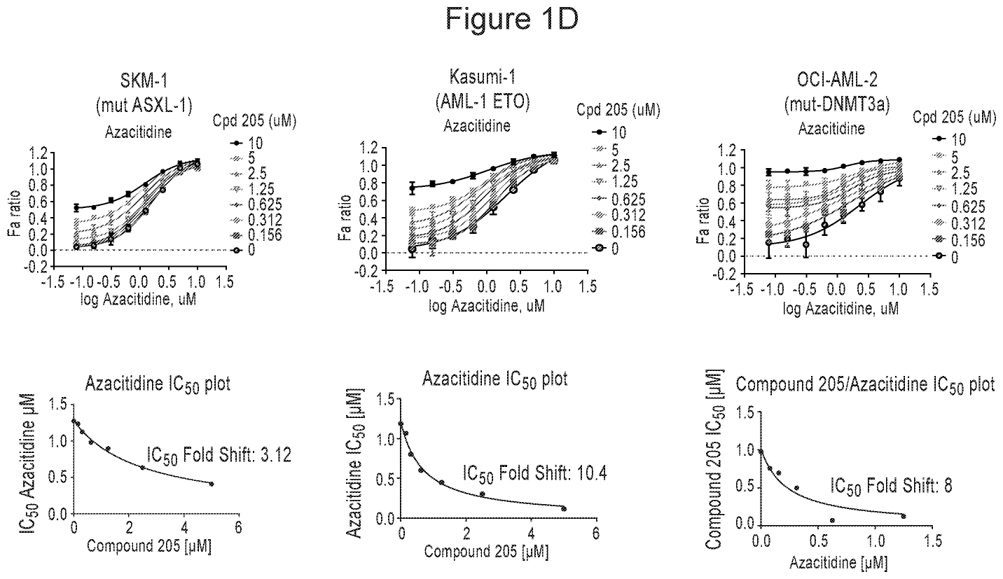

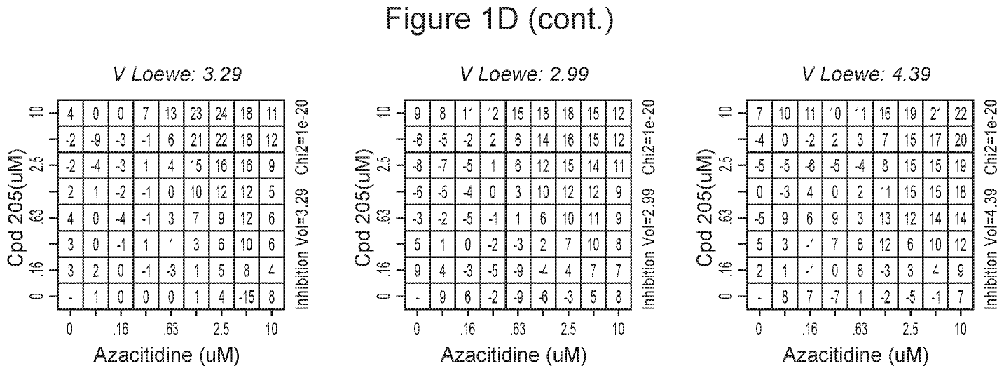

Pretreatment model: Various cell lines were seeded in flasks at densities that ensured log-linear growth rates for the duration of the assay. Flasks were dosed with 3 or 4 concentrations of Compound 205 (an EHMT2 inhibitor) in 3-fold dilutions and one additional flask was dosed with DMSO (vehicle) only at a final concentration of 0.1% v/v. Cultures were incubated in a humidified atmosphere of 5% CO₂ at 37° C. for 4 days. On Day 4 cells were spun down, resuspended in fresh medium, counted and diluted to the original cell density in new flasks. Cell cultures were re-dosed with Compound 205 and incubated for an additional 3 days. Cells were then spun down on Day 7, re-suspended in fresh medium and plated to assay-ready 384 well plates with an automated multichannel dispenser. The assay-ready 384-well plates contained 3-fold serial dilutions in triplicated of the combination partner compounds alone or in combination with the corresponding pre-treatment concentration of Compound 205. Compounds listed in Table 7 were dispensed with a HP-D300 nanoliter dispenser (Tecan, Mannedorf, Switzerland), with each plate containing 8 combination partners. Plates were then incubated for an additional three or seven days as noted in FIG. 1D to follow a 7+3 or 7+7 model (cell lines with slow growth characteristics were tested in a 7+7 model). Quantification of proliferation through measurement of cellular adenosine triphosphate (ATP) was performed via a luminescent cell viability assay and read on a plate reader with luminescence module. Quantification of synergy was performed with Chalice software (Horizon™, Cambridge, UK) using the Loewe Additivity model and calculating the Loewe Volume or V Loewe (Lehar J et al. (2007) Chemical combination effects predict connectivity in biological systems, Molecular Systems Biology 3:80). Examples of dose matrix, Loewe excess model, synergy quantification by V Loewe and isobologram are shown in FIG. 1A. Dose response curves of Fa (fraction affected) vs log concentration of compound in the presence or absence of a combination partner, IC₅₀ of one compound vs concentration of combination partner plots shown in FIG. 1A were generated with Graphpad Prism software.

Fa was calculated with the formula:

Fa=1−(Luminescence of test compound/Luminesence of untreated control)

Examples of pretreatment model studies are shown in FIGS. 1B and 1C.

Cotreatment Model: various cell lines were directly plated to 384 well plates with an automated multichannel dispenser onto plates containing 3-fold serial dilutions of combination partners, and Compound 205 in a matrix format in quadruplicates. Cells were incubated for seven days under humidified atmosphere of 5% CO₂ at 37° C. The final concentration of DMSO (vehicle) in the assay was 0.1% v/v. Quantification of proliferation through measurement of cellular adenosine triphosphate (ATP) was performed via a luminescent cell viability assay. Plates were read in a plate reader with luminescence module. Quantification of synergy was performed using the Loewe Additivity model and calculating the Loewe Volume (V Loewe) with the Chalice Software (Horizon) and dose response curves and IC₅₀ vs concentration plots were generated with Graphpad Prism software.

Examples of co-treatment studies are shown in FIG. 1D. The results of the combination studies of Compound 205 with other therapies in the pretreatment and cotreatment models described above are summarized in Table 8A and Table 8B.

TABLE 7

| Rationale | Modality | Drug name |
|---|---|---|
| AML Standard of Care | Antimetabolite | Cytarabine (Ara-C) |
| | Topoisomerase II inhibitor | Daunorubicin |
| Epigenetic drugs | DNA Hypomethylating agent | Azacitidine |
| | | Decitabine |
| | HDAC inhibitors | Pracinostat |
| | | Panobinostat |
| | EZH2 inhibitor | Tazemetostat |
| | DOT1L inhibitor | Pinometostat |
| | IDH1/2 inhibitors | AG-120 |
| | | AG-220 |
| Targeted Therapies | Differentiation agent | ATRA |
| | FLT3 inhibitors | Gilteritinib |
| | | Midostaurin |
| | BCL2 inhibitor | Venetoclax |

TABLE 8A

| | | Cell line | | | | | |
|---|---|---|---|---|---|---|---|
| | | AML-193 | AP-1060 | EOL-1 | HL-60 | Kasumi-1 | ML-2 |
| | | Model tested | | | | | |
| | | 7 + 7 | 7 + 7 | 7 + 3 | 7 + 3 | 7 + 7 | 7 + 3 |
| | | Genetic alterations | | | | | |
| | | | | | | | MLL- |
| | | | PML- | | MYC | AML1- | AF6 |
| | | | RARA | MLL-PTD | amplification | ETO | TP53 |
| Combination | Cytarabine | B | C | A | C | B | C |
| partner | Daunorubicin | C | C | C | C | C | B |
| | ATRA | D | C | C | A | A | A |
| | Azacitidine | B | C | C | C | B | C |
| | Decitabine | A | C | C | A | A | A |
| | Pinometostat | F | C | A | F | A | F |
| | (EPZ-5676) | | | | | | |
| | Tazemetostat | F | B | B | F | C | F |
| | (EPZ-6438) | | | | | | |
| | Gilteritinib | C | B | B | A | B | A |
| | Midostaurin | C | B | A | C | B | C |
| | Panobinostat | C | B | A | B | B | B |
| | Pracinostat | C | B | A | B | B | C |
| | Venetoclax | E | A | B | A | A | E |

Cell line

| | | | MOLM- | | OCI- | OCI- | |
|---|---|---|---|---|---|---|---|
| | | MOLM-13 | 16 | NOMO-1 | AML-2 | AML-3 | SKM-1 |
| | | Model tested | | | | | |
| | | 7 + 3 | 7 + 3 | 7 + 3 | 7 + 3 | 7 + 3 | 7 + 3 |
| | | Genetic alterations | | | | | |
| | | MLL-AF9 | | MLL-AF9 | | DNMT3A | |
| | | FLIT3-ITD | | KRAS | DNMT3 | NPM1 | ASXL1 |
| Combination | Cytarabine | C | E | E | C | C | C |
| partner | Daunorubicin | C | C | E | C | C | C |
| | ATRA | C | D | C | A | A | B |
| | Azacitidine | C | C | B | B | C | C |
| | Decitabine | C | D | C | A | C | B |
| | Pinometostat | A | F | F | B | F | B |
| | (EPZ-5676) | | | | | | |
| | Tazemetostat | F | F | F | B | F | C |
| | (EPZ-6438) | | | | | | |
| | Gilteritinib | A | C | B | B | C | B |
| | Midostaurin | A | E | E | B | C | C |
| | Panobinostat | C | B | C | A | B | B |
| | Pracinostat | C | C | C | B | C | C |
| | Venetoclax | A | E | C | B | D | B |

TABLE 8B

| | | Cell line | | | | | |
|---|---|---|---|---|---|---|---|
| | | AML-193 | AP-1060 | EOL-1 | HL-60 | Kasumi-1 | |
| | | Genetic Alterations | | | | | |
| | | | PML- | | | AML1- | |
| | | | RARA | MLL-PTD | MYCamp | ETO | |
| Combination | Azacitidine | A | C | C | A | A | |
| partner | Decitabine | A | A | C | A | A | |
| | Pinometostat | D | A | A | E | A | |
| | (EPZ-5676) | | | | | | |
| | Tazemetostat | E | A | B | E | B | |
| | (EPZ-6438) | | | | | | |
| | Cytarabine | | | A | | B | |
| | Atra | | | A | | C | |
| | Pracinostat | | | D | | | |
| | Venetoclax | | | C | | A | |
| | | Cell line | | | | | |
| | | | | | OCI- | OCI- | |
| | | ML-2 | MOLM-13 | MOLM-16 | AML2 | AML-3 | SKM-1 |
| | | Genetic Alterations | | | | | |
| | | MLL-AF6 | MLL-AF9 | | | DNMT3A | |
| | | TP53 | FLIT3-ITD | | DNMT3 | NPM1 | ASXL1 |
| Combination | Azacitidine | B | C | C | A | D | A |
| partner | Decitabine | A | B | E | A | C | A |
| | Pinometostat | A | A | A | A | D | A |
| | (EPZ-5676) | | | | | | |
| | Tazemetostat | E | E | C | A | E | C |
| | (EPZ-6438) | | | | | | |
| | Cytarabine | | | | C | D | A |
| | Atra | | | | A | A | A |
| | Pracinostat | | | | A | D | |
| | Venetoclax | | | | C | C | C |

| A | B | C | D | E | F |
|---|---|---|---|---|---|
| Synergy | Slight Synergy | Additivity | Slight Antagonism | Antagonism | No effect |
| Loewe | Loewe volume | Loewe volume | Loewe volume | Loewe | Neither agent or |
| volume >2 | between 1.0 and | between −1 and 0.9 | between −1.1 and 2 | volume <−2 | combination of |
| | 1.9 | | | | the two reached |
| | | | | | 50% inhibition |

### Example 4: In Vitro Single-Agent Studies of EHMT2 Inhibitor Compound

A screen of 284 cell lines to assess the antiproliferative effect of EHMT2 inhibition was conducted by treating cell lines in 384-well format with a half-log step dilutions of Compound 205 over 10 concentrations with a maximum concentration of 0.1% v/v DMSO. Cells were plated on Day 0 and treated with compound on Day 1. Culture medium was replaced on day 7 and cells redosed. After 10-day incubation, cells were fixed and stained with nuclear dye. Automated fluorescence microscopy was carried out using a Molecular Devices ImageXpress Micro XL high-content imager, and images were collected with a 4× objective. 16-bit TIFF images were acquired and analyzed with MetaXpress 5.1.0.41 software. Cell proliferation was measured by the fluorescence intensity of the incorporated nuclear dye. Cell count IC₅₀ is the test compound concentration at 50% of maximal response of the untreated control.

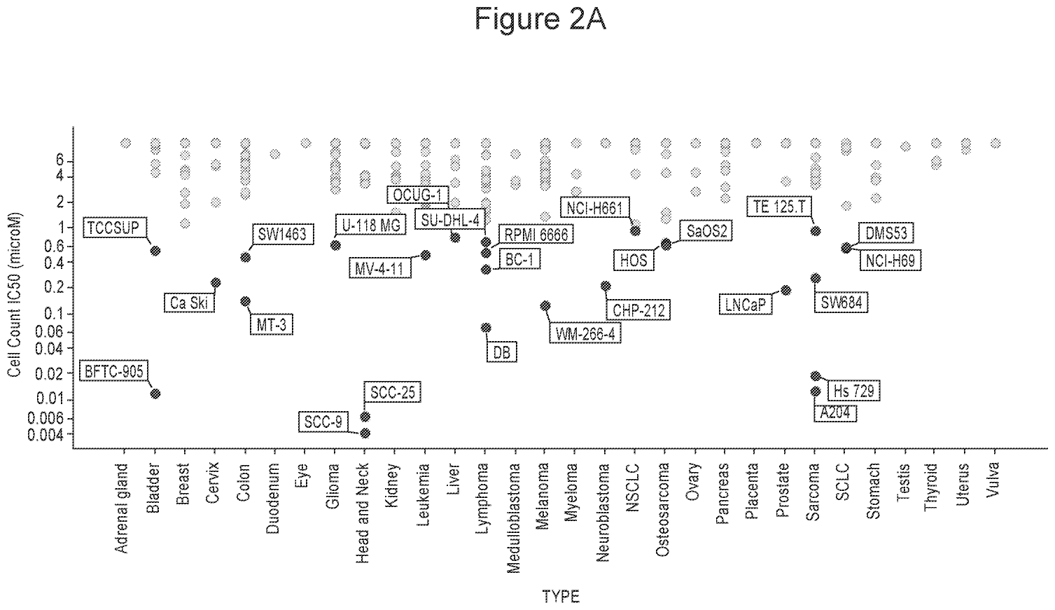

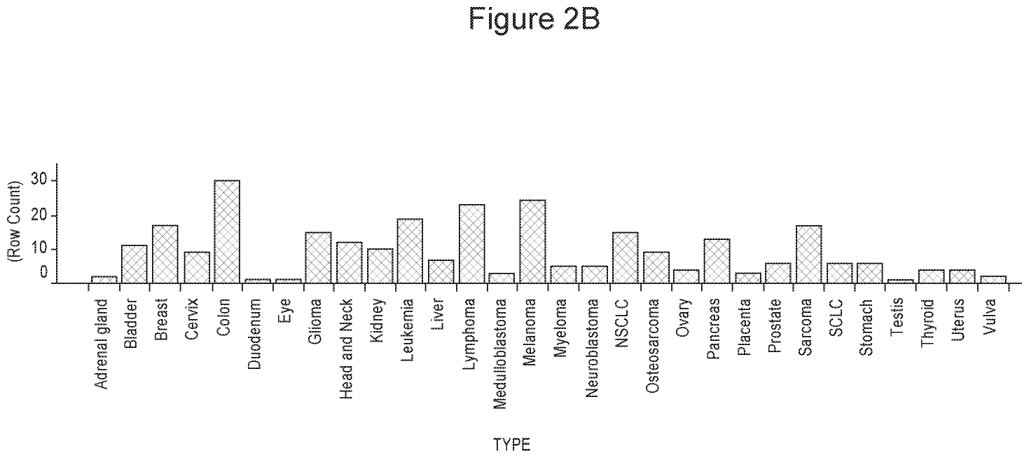

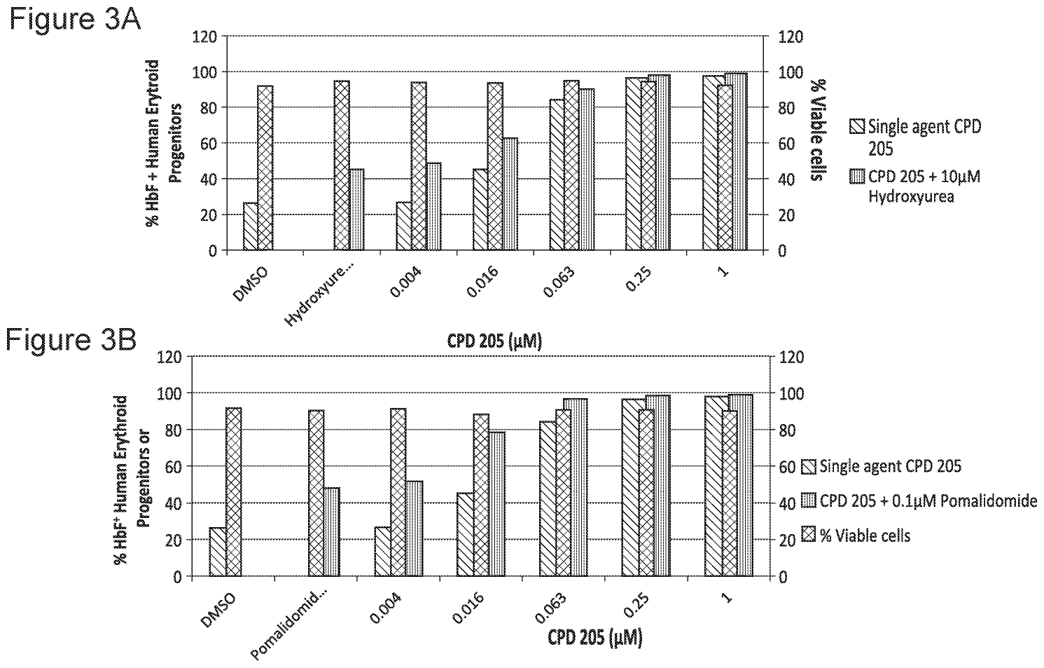

The results of the single-agent studies of EHMT2 Inhibitor, Compound 205, are summarized in FIGS. 2 and 3. FIG. 2A shows a plot of Cell Count IC₅₀ in micromolar (microM) concentration values for all cell lines vs type of cancer. Cell lines with Cell Count IC₅₀ less than 1 uM are labeled on the graph. The number of cell lines within each type of cancer are shown as a bar graph in FIG. 2B. Table 9 shows the results for the 284 cell lines (“A” means IC₅₀<10 nM; “B” means IC₅₀ ranging between 10 nM and <100 nM; “C” means IC₅₀ ranging between 100 nM and <1 μM; “D” means IC₅₀ ranging between 1 μM and 10 μM; “E” means IC₅₀ >10 μM).

TABLE 9
Cell count results for the 284 tested cell lines.

| | | | Cell Count |
|---|---|---|---|
| Cell Line | Tissue | Type | IC50 (μM) |
| SCC-9 | Head and Neck | Head and Neck | A |
| SCC-25 | Head and Neck | Head and Neck | A |
| BFTC-905 | Bladder | Bladder | B |
| A204 | Soft & Connective Tissue | Sarcoma | B |
| Hs 729 | Soft & Connective Tissue | Sarcoma | B |
| DB | Hematopoietic | Lymphoma | B |
| WM-266-4 | Skin | Melanoma | C |
| MT-3 | Colon | Colon | C |
| LNCaP | Prostate | Prostate | C |
| CHP-212 | Central Nervous System | Neuroblastoma | C |
| Ca Ski | Female GU | Cervix | C |
| SW684 | Soft & Connective Tissue | Sarcoma | C |
| BC-1 | Hematopoietic | Lymphoma | C |
| SW1463 | Colon | Colon | C |
| MV-4-11 | Hematopoietic | Leukemia | C |
| RPMI 6666 | Hematopoietic | Lymphoma | C |
| TCCSUP | Bladder | Bladder | C |
| DMS53 | Lung | SCLC | C |
| NCI-H69 | Lung | SCLC | C |
| U-118 MG | Central Nervous System | Glioma | C |
| SaOS2 | Bone | Osteosarcoma | C |
| HOS | Bone | Osteosarcoma | C |
| SU-DHL-4 | Hematopoietic | Lymphoma | C |
| OCUG-1 | Liver | Liver | C |
| NCI-H661 | Lung | NSCLC | C |
| TE 125.T | Soft & Connective Tissue | Sarcoma | C |
| COR-L105 | Lung | NSCLC | D |
| CAMA-1 | Breast | Breast | D |
| NAMALWA | Hematopoietic | Lymphoma | D |
| SJSA1 | Bone | Osteosarcoma | D |
| MeWo | Skin | Melanoma | D |
| ACHN | Kidney | Kidney | D |
| Hs 445 | Hematopoietic | Lymphoma | D |
| MG-63 | Bone | Osteosarcoma | D |
| ARH-77 | Hematopoietic | Myeloma | D |
| TF-1 | Hematopoietic | Leukemia | D |
| RS4;11 | Hematopoietic | Leukemia | D |
| SR | Hematopoietic | Lymphoma | D |
| NALM-6 | Hematopoietic | Leukemia | D |
| DMS114 | Lung | SCLC | D |
| SK-N-AS | Central Nervous System | Neuroblastoma | D |
| MOLT-16 | Hematopoietic | Leukemia | D |
| MDA MB 468 | Breast | Breast | D |
| SUP-T1 | Hematopoietic | Lymphoma | D |
| DoTc2 4510 | Female GU | Cervix | D |
| SU-DHL-10 | Hematopoietic | Lymphoma | D |
| HUH-6 Clone 5 | Liver | Liver | D |
| KATO III | Stomach | Stomach | D |
| HPAF-II | Pancreas | Pancreas | D |
| Jurkat | Hematopoietic | Leukemia | D |
| Colo 201 | Colon | Colon | D |
| SK-BR-3 | Breast | Breast | D |
| LS123 | Colon | Colon | D |
| RPMI 8226 | Hematopoietic | Myeloma | D |
| PA-1 | Female GU | Ovary | D |
| SKO-007 | Hematopoietic | Myeloma | D |
| SNB-19 | Central Nervous System | Glioma | D |
| Daudi | Hematopoietic | Lymphoma | D |
| AsPC-1 | Pancreas | Pancreas | D |
| SK-MEL-28 | Skin | Melanoma | D |
| COLO 829 | Skin | Melanoma | D |
| BV-173 | Hematopoietic | Leukemia | D |
| SJRH30 | Soft & Connective Tissue | Sarcoma | D |
| D283 Med | Central Nervous System | Medulloblastoma | D |
| Thp 1 | Hematopoietic | Leukemia | D |
| OE21 | Head and Neck | Head and Neck | D |
| FaDu | Head and Neck | Head and Neck | D |
| U-138MG | Central Nervous System | Glioma | D |
| HT | Hematopoietic | Lymphoma | D |
| SNU-423 | Liver | Liver | D |
| A172 | Central Nervous System | Glioma | D |
| Hs 683 | Central Nervous System | Glioma | D |
| JeKo-1 | Hematopoietic | Lymphoma | D |
| 22Rv1 | Prostate | Prostate | D |
| Hs 611.T | Hematopoietic | Lymphoma | D |
| SNU-C2B | Colon | Colon | D |
| Daoy | Central Nervous System | Medulloblastoma | D |
| A2058 | Skin | Melanoma | D |
| RKO-AS45-1 | Colon | Colon | D |
| SNU-5 | Stomach | Stomach | D |
| MOLT-3 | Hematopoietic | Leukemia | D |
| LS513 | Colon | Colon | D |
| SK-PN-DW | Soft & Connective Tissue | Sarcoma | D |
| SU-DHL-8 | Hematopoietic | Lymphoma | D |
| C32TG | Skin | Melanoma | D |
| MES-SA | Soft & Connective Tissue | Sarcoma | D |
| Caki-1 | Kidney | Kidney | D |
| G-402 | Kidney | Kidney | D |
| A388 | Skin | Head and Neck | D |
| EM-2 | Hematopoietic | Leukemia | D |
| DOHH-2 | Hematopoietic | Lymphoma | D |
| SNU-16 | Stomach | Stomach | D |
| DBTRG-05MG | Central Nervous System | Glioma | D |
| G-361 | Skin | Melanoma | D |
| CML-T1 | Hematopoietic | Leukemia | D |
| A-704 | Kidney | Kidney | D |
| Detroit 562 | Head and Neck | Head and Neck | D |
| Colo 205 | Colon | Colon | D |
| Cal 27 | Head and Neck | Head and Neck | D |
| RD | Soft & Connective Tissue | Sarcoma | D |
| SW403 | Colon | Colon | D |
| MDA MB 453 | Breast | Breast | D |
| 769-P | Kidney | Kidney | D |
| CA46 | Hematopoietic | Lymphoma | D |
| A427 | Lung | NSCLC | D |
| SK-MEL-3 | Skin | Melanoma | D |
| MHH-PREB-1 | Hematopoietic | Leukemia | D |
| U266B1 | Hematopoietic | Myeloma | D |
| TE 381.T | Soft & Connective Tissue | Sarcoma | D |
| KHOS-240S | Bone | Osteosarcoma | D |
| CaOV3 | Female GU | Ovary | D |
| HT-1197 | Bladder | Bladder | D |
| SH-4 | Skin | Melanoma | D |
| C32 | Skin | Melanoma | D |
| BT474 | Breast | Breast | D |
| TUR | Hematopoietic | Lymphoma | D |
| ST486 | Hematopoietic | Lymphoma | D |
| PSN-1 | Pancreas | Pancreas | D |
| U-87 MG | Central Nervous System | Glioma | D |
| AUS65 | Breast | Breast | D |
| SW1417 | Colon | Colon | D |
| Hs 936.T(C1) | Skin | Melanoma | D |
| Hs 695T | Skin | Melanoma | D |
| Hs 821.T | Soft & Connective Tissue | Sarcoma | D |
| MS751 | Female GU | Cervix | D |
| SW1783 | Central Nervous System | Glioma | D |
| A498 | Kidney | Kidney | D |
| RPMI-7951 | Skin | Melanoma | D |
| HuCCT1 | Liver | Liver | D |
| MEG01 | Hematopoietic | Leukemia | D |
| AGS | Stomach | Stomach | D |
| BHT-101 | Endocrine | Thyroid | D |
| HuP-T4 | Pancreas | Pancreas | D |
| RKOE6 | Colon | Colon | D |
| Hs 294T | Skin | Melanoma | D |
| SiHa | Female GU | Cervix | D |
| DK-MG | Central Nervous System | Glioma | D |
| WiDr | Colon | Colon | D |
| SCaBER | Bladder | Bladder | D |
| NCI-H747 | Colon | Colon | D |
| LS411N | Colon | Colon | D |
| SW837 | Colon | Colon | D |
| A101D | Skin | Melanoma | D |
| TT | Endocrine | Thyroid | D |
| LS-174T | Colon | Colon | D |
| Hs 688(A).T | Skin | Melanoma | D |
| HLF | Liver | Liver | D |
| HT-29 | Colon | Colon | D |
| SW872 | Soft & Connective Tissue | Sarcoma | D |
| MDA MB 231 | Breast | Breast | D |
| Ramos (RA 1) | Hematopoietic | Lymphoma | D |
| SW620 | Colon | Colon | D |
| RKO | Colon | Colon | D |
| U2OS | Bone | Osteosarcoma | D |
| D341 Med | Central Nervous System | Medulloblastoma | D |
| EB2 | Hematopoietic | Lymphoma | D |
| HuTu 80 | Duodenum | Duodenum | D |
| SW48 | Colon | Colon | D |
| SW1088 | Central Nervous System | Glioma | D |
| Caki-2 | Kidney | Kidney | D |
| K562 | Hematopoietic | Leukemia | D |
| CCF-STTG1 | Central Nervous System | Glioma | D |
| PANC-1 | Pancreas | Pancreas | D |
| NCIH446 | Lung | SCLC | D |
| HEC-1-A | Female GU | Uterus | D |
| SKMES1 | Lung | NSCLC | D |
| 647-V | Bladder | Bladder | D |
| SK-MEL-1 | Skin | Melanoma | D |
| SW900 | Lung | SCLC | D |
| A375 | Skin | Melanoma | D |
| NTERA-2 cl.D1 | Testis | Testis | D |
| J82 | Bladder | Bladder | D |
| BxPC-3 | Pancreas | Pancreas | D |
| COR-L23 | Lung | NSCLC | D |
| Mia PaCa-2 | Pancreas | Pancreas | D |
| SW480 | Colon | Colon | D |
| A431 | Skin | Head and Neck | D |
| UM-UC-3 | Bladder | Bladder | D |
| 5637 | Bladder | Bladder | F |
| 639-V | Bladder | Bladder | E |
| 786-O | Kidney | Kidney | E |
| A-253 | Head and Neck | Head and Neck | E |
| A549 | Lung | NSCLC | E |
| A-673 | Soft & Connective Tissue | Sarcoma | E |
| A7 | Skin | Melanoma | E |
| AN3 CA | Female GU | Uterus | E |
| BE(2)C | Central Nervous System | Neuroblastoma | E |
| BeWo | Placenta | Placenta | E |
| BM-1604 | Prostate | Prostate | E |
| BPH1 | Prostate | Prostate | E |
| BT20 | Breast | Breast | E |
| BT-549 | Breast | Breast | E |
| C-33A | Female GU | Cervix | E |
| C-4 II | Female GU | Cervix | E |
| CAL-62 | Endocrine | Thyroid | E |
| Calu1 | Lung | NSCLC | E |
| Calu6 | Lung | NSCLC | E |
| Capan-1 | Pancreas | Pancreas | E |
| Capan-2 | Pancreas | Pancreas | E |
| CCRFCEM | Hematopoietic | Leukemia | E |
| CEM-C1 | Hematopoietic | Leukemia | E |
| CFPAC-1 | Pancreas | Pancreas | E |
| CGTH-W-1 | Endocrine | Thyroid | E |
| ChaGoK1 | Lung | NSCLC | E |
| CHL-1 | Skin | Melanoma | E |
| Colo 320 HSR | Colon | Colon | E |
| Colo 320DM | Colon | Colon | E |
| DLD-1 | Colon | Colon | E |
| DU145 | Prostate | Prostate | E |
| EB-3 | Hematopoietic | Lymphoma | E |
| EFM-19 | Breast | Breast | E |
| G-292, clone | Bone | Osteosarcoma | E |
| A141B1 | | | |
| G-401 | Kidney | Kidney | E |
| H4 | Central Nervous System | Glioma | E |
| HCT-116 | Colon | Colon | E |
| HCT-15 | Colon | Colon | E |
| HCT-8 | Colon | Colon | E |
| HEL-92-1-7 | Hematopoietic | Leukemia | E |
| HeLa | Female GU | Cervix | E |
| HepG2 | Liver | Liver | E |
| HLE | Liver | Liver | E |
| HMCB | Skin | Melanoma | E |
| Hs 229.T | Lung | NSCLC | E |
| Hs 578T | Breast | Breast | E |
| HS 746T | Stomach | Stomach | E |
| Hs 766T | Pancreas | Pancreas | E |
| Hs 852.T | Skin | Melanoma | E |
| Hs 888.Sk | Bone | Osteosarcoma | E |
| Hs 934.T | Skin | Melanoma | E |
| HT-1080 | Soft & Connective Tissue | Sarcoma | E |
| HT1376 | Bladder | Bladder | E |
| HT-3 | Female GU | Cervix | E |
| IM-9 | Hematopoietic | Myeloma | E |
| JAR | Placenta | Placenta | E |
| JEG-3 | Placenta | Placenta | E |
| Jiyoye | Hematopoietic | Lymphoma | E |
| KLE | Female GU | Uterus | E |
| KPL-1 | Breast | Breast | E |
| KU812 | Hematopoietic | Leukemia | E |
| LS1034 | Colon | Colon | E |
| M059J | Central Nervous System | Glioma | E |
| MALME3M | Skin | Melanoma | E |
| MCF7 | Breast | Breast | E |
| MC-IXC | Central Nervous System | Neuroblastoma | E |
| MDA-MB-415 | Breast | Breast | E |
| MDA-MB-436 | Breast | Breast | E |
| ME-180 | Female GU | Cervix | E |
| MX1 | Hematopoietic | Leukemia | E |
| NCI-H292 | Lung | NSCLC | E |
| NCI-H295R | Endocrine | Adrenal gland | E |
| NCIH441 | Lung | NSCLC | E |
| NCI-H460 | Lung | NSCLC | E |
| NCI-H508 | Colon | Colon | E |
| NCI-H520 | Lung | NSCLC | E |
| NCI-H596 | Lung | NSCLC | E |
| OE19 | Head and Neck | Head and Neck | E |
| OE33 | Head and Neck | Head and Neck | E |
| OVCAR3 | Female GU | Ovary | E |
| PC-3 | Prostate | Prostate | E |
| PFSK-1 | Central Nervous System | Glioma | E |
| Raji | Hematopoietic | Lymphoma | E |
| RL95-2 | Female GU | Uterus | E |
| SCC-4 | Head and Neck | Head and Neck | E |
| SHP-77 | Lung | SCLC | E |
| SK-LMS-1 | Soft & Connective Tissue | Sarcoma | E |
| SK-NEP-1 | Kidney | Kidney | E |
| SK-N-FI | Central Nervous System | Neuroblastoma | E |
| SKOV3 | Female GU | Ovary | E |
| SK-UT-1 | Soft & Connective Tissue | Sarcoma | E |
| SNU-1 | Stomach | Stomach | E |
| SU.86.86 | Pancreas | Pancreas | E |
| SW-13 | Endocrine | Adrenal gland | E |
| SW1353 | Bone | Osteosarcoma | E |
| SW948 | Colon | Colon | E |
| SW954 | Female GU | Vulva | E |
| SW962 | Female GU | Vulva | E |
| SW982 | Soft & Connective Tissue | Sarcoma | E |
| T24 | Bladder | Bladder | E |
| T47D | Breast | Breast | E |
| T98G | Central Nervous System | Glioma | E |
| VA-ES-BJ | Soft & Connective Tissue | Sarcoma | E |
| Y79 | Eve | Eye | E |
| YAPC | Pancreas | Pancreas | E |
| ZR-75-1 | Breast | Breast | E |

### Example 5: Human CD34+ Progenitors Assay to Test for Fetal Hemoglobin Induction

An in-vitro system to test the ability of compound to induce fetal hemoglobin expression was developed. This system used Human CD34+ progenitor cells freshly isolated from healthy donors blood collections. Peripheral blood mononuclear cells (PBMCs) were isolated from whole blood by density gradient centrifugation using SepMatem-50 and Lymphoprep™ from Stem Cell Technologies. CD34⁺ hematopoietic progenitor cells (HSPCs) were isolated from PBMCs by magnetic separation using Stem Cells Technologies CD34+ positive selection/isolation kit (Stem Cell Technologies, #18056). CD34 cells were cultured using a 2-phase 14 day culture system. During phase 1 (day 0 to day 7) cells were expanded. Expansion was followed by phase 2 (day 7 to day 14) where cells were differentiated toward the erythroid lineage. Compounds were added on day 1 and day 7 as 1000× stock after being diluted in dimethyl sulfoxide (DMSO) in a 3-fold series. Final DMSO concentration in the assay was 0.1%. At day 14 cells were harvested for Fluorescent Activated Cell Sorting (FACS) analysis and for HbF quantitation by Mass spectrometry. For cell surface and intracellular marker analysis by FACS, cells were fixed and stained with a cocktail of antibodies covering erythroid lineage markers, HbF and H3 and H3K9me2 (Table 11). Data was collected using Canto II flow cytometer (BD Biosciences) and the FACSDiva software. Data was analyzed using FlowJo software. % HbF⁺ cells and ratios H3/H3K9me2 intensities were calculated for CD71⁺/CD235a⁺ gated populations.

TABLE 10
Human CD34+ system culture conditions for Phase 1 and Phase 2

| | | | |
|---|---|---|---|
| | | Catalog | |
| Additive | Source | Number | [final] |

CD34+ Culture Conditions Phase 1

| | | | |
|---|---|---|---|
| IMDM | Thermofisher | 12440079 | 94% |
| Human Serum | | | 5% |
| Glutamax 100× | Thermofisher | 35050-061 | 1% |
| Holotransferrin | Sigma | T4132-1G | 330 ug/mL |
| Insulin | Sigma | I9278 | 10 ug/mL |
| Heparin | Sigma | 1304005 | 2 IU/mL |
| EPO | R&D | 287-TC | 0.5 U/mL |
| SCF | R&D | 255-SC- | 100 ng/mL |
| | | 010/CF | |
| IL-3 | R&D | 203-IL-010 | 5 ng/mL |
| Hydrocortisone | Sigma | H6909 | 1 μM |

CD34+ Culture Conditions Phase 2

| | | | |
|---|---|---|---|
| IMDM | Thermofisher | 12440079 | 94% |
| Human Serum | | | 5% |
| Glutamax 100× | Thermofisher | 35050-061 | 1% |
| Holotransferrin | Sigma | T4132-1G | 330 ug/mL |
| Insulin | Sigma | I9278 | 10 ug/mL |
| Heparin | Sigma | 1304005 | 2 IU/mL |
| EPO | R&D | 287-TC | 3 U/mL |
| SCF | R&D | 255-SC- | 100 ng/mL |
| | | 010/CF | |

TABLE 11
Antibodies cocktail for FACS analysis

| Antibody | Conjugate | Vendor | Cat No |
|---|---|---|---|
| CD34 | BV510 | Biolegend | 343528 |
| CD235a | BV421 | BD | 562938 |
| CD71 | PE-Cy7 | eBiosciences | 25071942 |
| CD45 | APC/fire 450 | Biolegend | 368518 |
| CD36 | Percp-Cy5.5 | BD | 561536 |
| H3 | A647 | CST | 12230S |
| HbF | PE | Invitrogen | MHFH04-4 |
| H3K9me2 | A488 | Abcam | Ab203850 |

### Example 6: HBF Inducers and Combination Studies for G9A Inhibitors

A list of pharmacological agents was evaluated for their potential to induce fetal Hemoglobin (HbF) in order to identify combination partners for our EHMT1/2 inhibitors. HbF can be induced by toxicity; therefore, the potential of the agents to induce HbF were evaluated in the context of cell viability. (Table 12). The EHMT1/2 inhibitor Compound 205 was evaluated in combination with a fixed dose of 10 μM Hydroxyurea and 0.1 μM Pomalidomide. A combination of 0.016 μM compound 205 and 10 μM Hydroxyurea showed a clear positive effect while maintaining cell viability >90%. 10 μM Hydroxyurea as a single agent was able to induce 01% HbF⁺ cells from 26% basal level to 45% while 0.016 μM compound 205 as a single agent induced to 45%. In combination these two agents were able to induced % HbF⁺ to 63% (FIG. 3A). A combination of 0.016 μM compound 205 and 0.1 μM Pomalidomide showed a clear positive effect while maintaining cell viability >90%. 0.1 μM Pomalidomide as a single agent was able to induce ° % HbF cells from 26% basal level to 48% while 0.016 μM compound 205 as a single agent induced to 45%. In combination these two agents were able to induce % HbF⁺ to 78% (FIG. 3B).

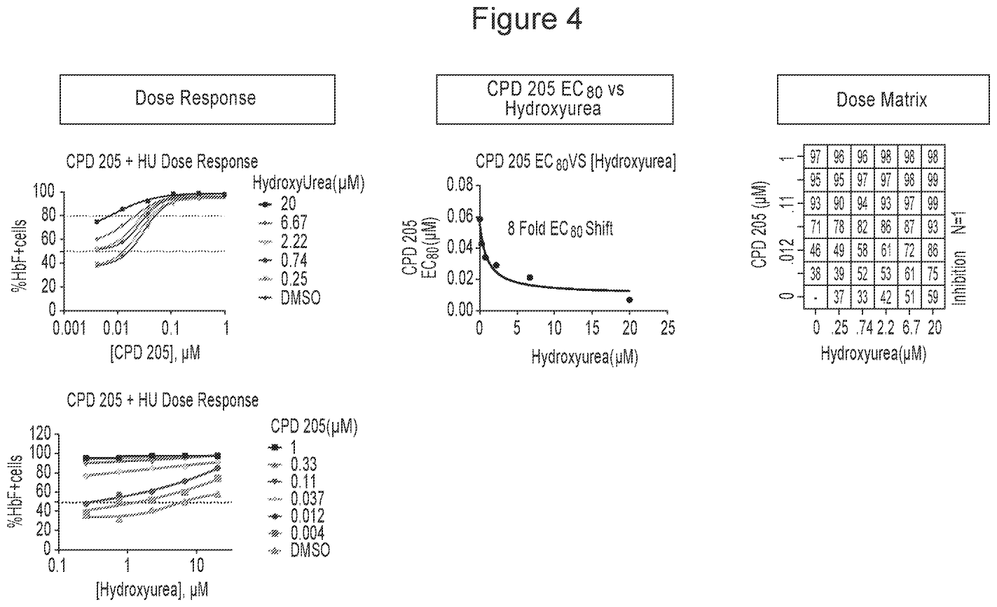

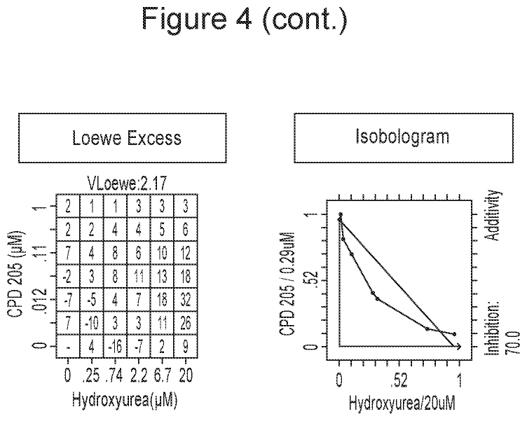

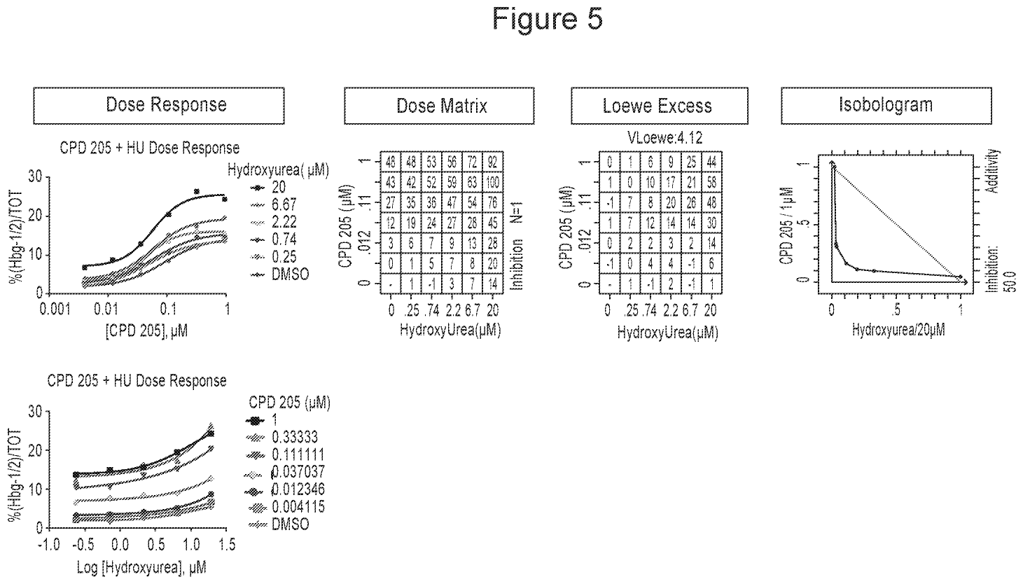

Hydroxyurea was also evaluated as single agent and in combination with the EHMT1/2 inhibitor compound 205 in a matrix format using CD34⁺ cells isolated from a pool of 5 healthy donors. Results showed the ability of these two agents to act synergistically using data from FACS analysis and MS quantification. (FIG. 4 and FIG. 5, respectively). It is noted that for Loewe excess determination in Chalice, data was normalized to the highest and lowest Hbγ induction observed under the conditions and dose ranges in the assay.

TABLE 12
Pharmacological agents with potential to induce Fetal hemoglobin expression

| | | Observed Induction of |
|---|---|---|
| | | % HbF + Human |
| Agent | Class | Erythroid Progenitors |
| Hydroxyurea | SOC for SCD | Yes |
| Entinostat | Pan-HDAC Inhibitor | Yes |
| Vorinostat | Pan-HDAC Inhibitor | No |
| Panobinostat | Pan-HDAC Inhibitor | No |
| Acethylon ACY-957 | HDAC 1/2 Inhibitor | Yes |
| BG-45 | HDAC 3 Inhibitor | Yes |
| Decitabine | DMNT1 Inhibitor | Yes |
| Desloratidine | Anti-histamine (Claritin) | No |
| Benzerazide | Decarboxilase Inhibitor | Very small |
| | (Parkinson) | |
| Pomalidomide | Immunomodulator | Yes |
| Metformin | Diabetes drug shown to be | Yes |
| | FOXO-3 Inducer | |
| PDE9 | Phosphodiesterase 9 Inhibitors | No |

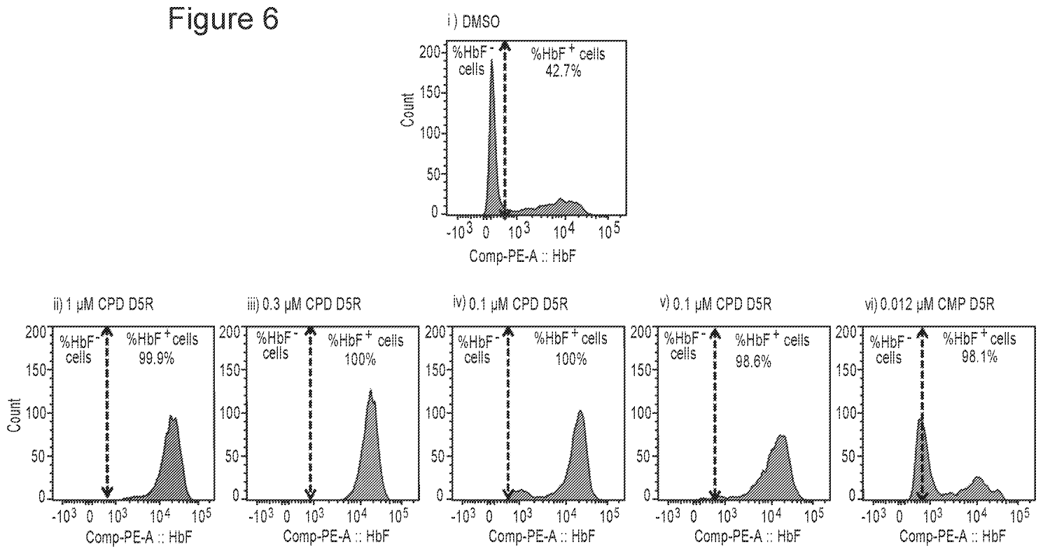

An agent can be defined as an HbF pan cellular inducer if it has the capability to induce the expression of HbF in all the cells of a treated population versus in a fraction of cells (heterocellular). For each treatment, HbF expressing cells (HbF⁺) were expressed as a percent of the total population and were defined as cells right of the threshold bar which was determined based on the DMSO control shown in (FIG. 6 (i)) (dotted lines). In FIG. 6 (i) are cells treated at 0.1% DMSO showed baseline levels of 42.7% of HbF expressing cells, in FIG. 6 (ii)-(vi) are cells treated with Compound D5R in a dose response manner. In this range of concentration, Compound D5R was able to sustain pan-cellularity effect of induction of HbF shown by % HbF+ cells >98.

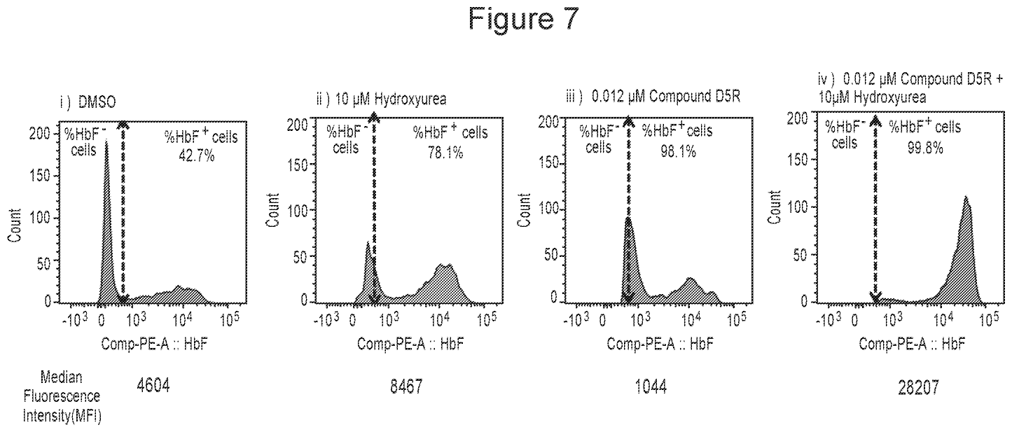

An agent can be defined as an HbF pan cellular inducer if it has the capability to induce the expression of HbF in all the cells of a treated population versus in a fraction of cells (heterocellular). For each treatment, HbF expressing cells (HbF⁺) were expressed as a percent of the total population and are defined as cells right of the threshold bar which was determined based on the DMSO control shown in (FIG. 7 (i)) (dotted lines). In FIG. 7 (i) are cells treated at 0.1% DMSO showed baseline levels of 42.7% of HbF expressing cells with a wide spread of MFI, in FIG. 7 (ii) are cells treated at 10 μM Hydroxyurea showed 78.1% of HbF expressing cells with a wide spread of MFI but with most of the positive cells concentrated at ˜10(4) Fluorescence Intensity, in FIG. 7 (iii) are cells treated at 0.012 μM Compound D5R showed 98.1% of HbF expressing cells with a wide spread of MFI but with most of the positive cells concentrated at ˜10(3) Fluorescence Intensity, in FIG. 7 (iv) are cells treated at 10 μM Hydroxyurea in combination with 0.012 μM Compound D5R showed 99.8%% of HbF expressing cells with a strong single peak centered at ˜3×10(4) Fluorescence Intensity

Aspects of this disclosure can be embodied in other specific forms without departing from the spirit or essential characteristics thereof. The foregoing embodiments are therefore to be considered in all respects illustrative rather than limiting on the disclosed inventive concepts described herein. The scope of the disclosure is thus indicated by the appended claims rather than by the foregoing description, and all changes that come within the meaning and range of equivalency of the claims are intended to be embraced therein.

1. A method of preventing or treating a blood disorder, the method comprising administering to a subject in need thereof a therapeutically effective amount of an EHMT2 inhibitor.

2. The method of claim 1, wherein the blood disorder is Acute lymphoblastic leukemia (ALL), Acute myeloid leukemia (AML), Amyloidosis, Anemia, Aplastic anemia, Bone marrow failure syndromes, Chronic lymphocytic leukemia (CLL), Chronic myeloid leukemia (CML), Deep vein thrombosis (DVT), Diamond-Blackfan anemia, Dyskeratosis congenita (DKC), Eosinophilic disorder, Essential thrombocythemia, Fanconi anemia, Gaucher disease, Hemochromatosis, Hemolytic anemia, Hemophilia, Hereditary spherocytosis, Hodgkin's lymphoma, Idiopathic thrombocytopenic purpura (ITP), Inherited bone marrow failure syndromes, Iron-deficiency anemia, Langerhans cell histiocytosis, Large granular lymphocytic (LGL) leukemia, Leukemia, Leukopenia, Mastocytosis, Monoclonal gammopathy, Multiple myeloma, Myelodysplastic syndromes (MDS), Myelofibrosis, Myeloproliferative neoplasms (MPN), Non-Hodgkin's lymphoma, Paroxysmal nocturnal hemoglobinuria (PNH), Pernicious anemia (B12 deficiency), Polycythemia vera, Porphyria, Post-transplant lymphoproliferative disorder (PTLD), Pulmonary embolism (PE), Shwachman-Diamond syndrome (SDS), Sickle-cell disease (SCD), Thalassemia, Thrombocytopenia, Thrombotic thrombocytopenic purpura (TTP), Venous thromboembolism, Von Willebrand disease, or Waldenstrom's macroglobulinemia (lymphoplasmacytic lymphoma).

3. The method of claim 1, wherein the EHMT2 inhibitor is a compound of Formula (I): 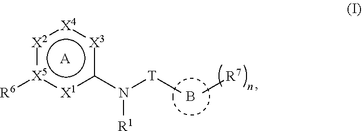
or a tautomer thereof, or a pharmaceutically acceptable salt of the compound or the tautomer, wherein
ring A is phenyl or a 5- or 6-membered heteroaryl;
X¹ is N, CR², or NR²⁺ as valency permits;
X² is N, CR³, or NR³⁺ as valency permits;
X³ is N, CR⁴, or NR⁴⁺ as valency permits;
X⁴ is N or CR⁵, or X⁴ is absent such that ring A is a 5-membered heteroaryl containing at least one N atom;
X⁵ is C or N as valency permits;
B is absent or a ring structure selected from the group consisting of C₆-C₁₀ aryl, C₃-C₁₀ cycloalkyl, 5- to 10-membered heteroaryl, and 4- to 12-membered heterocycloalkyl containing 1-4 heteroatoms selected from N, O, and S;
T is a bond or C₁-C₆ alkylene, C₂-C₆ alkenylene, or C₂-C₆ alkynylene linker optionally substituted with one or more of halo, cyano, hydroxyl, oxo, or C₁-C₆ alkoxy when B is present; or T is H and n is 0 when B is absent; or T is C₁-C₆ alkyl optionally substituted with (R⁷)ₙ when B is absent; or when B is absent, T and R¹ together with the atoms to which they are attached optionally form a 4-7 membered heterocycloalkyl or 5-6 membered heteroaryl, each of which is optionally substituted with (R⁷)ₙ;
R¹ is H or C₁-C₄ alkyl;
each of R², R³, and R⁴, independently is selected from the group consisting of H, halo, cyano, C₁-C₆ alkoxyl, C₆-C₁₀ aryl, NR^{a}R^{b}, C(O)NR^{a}R^{b}, NR^{a}C(O)R^{b}, C₃-C₈ cycloalkyl, 4- to 7-membered heterocycloalkyl, 5- to 6-membered heteroaryl, and C₁-C₆ alkyl, wherein C₁-C₆ alkoxyl and C₁-C₆ alkyl are optionally substituted with one or more of halo, OR, or NR^{a}R^{b}, in which each of R^{a} and R^{b} independently is H or C₁-C₆ alkyl, or R³ is -Q¹-T¹, in which Q¹ is a bond or C₁-C₆ alkylene, C₂-C₆ alkenylene, or C₂-C₆ alkynylene linker optionally substituted with one or more of halo, cyano, hydroxyl, oxo, or C₁-C₆ alkoxyl, and T¹ is H, halo, cyano, NR⁸R⁹, C(O)NR⁸R⁹, OR⁸, OR⁹, or R^{S1}, in which R^{S1} is C₃-C₈ cycloalkyl, phenyl, 4- to 12-membered heterocycloalkyl containing 1-4 heteroatoms selected from N, O, and S, or a 5- or 6-membered heteroaryl and R^{S1} is optionally substituted with one or more of halo, C₁-C₆ alkyl, hydroxyl, oxo, —C(O)R⁹, —SO₂R⁸, —SO₂N(R⁸)₂, —NR⁸C(O)R⁹, amino, mono- or di-alkylamino, or C₁-C₆ alkoxyl; or when ring A is a 5-membered heteroaryl containing at least one N atom, R⁴ is a spiro-fused 4- to 12-membered heterocycloalkyl containing 1-4 heteroatoms selected from N, O, and S;
each of R^{2′}, R^{3′} and R^{4′} independently is H or C₁-C₃ alkyl;
R⁵ is selected from the group consisting of H, F, Br, cyano, C₁-C₆ alkoxyl, C₆-C₁₀ aryl, NR^{a}R^{b}, C(O)NR^{a}R^{b}, NR^{a}C(O)R^{b}, C₃-C₈ cycloalkyl, 4- to 12-membered heterocycloalkyl containing 1-4 heteroatoms selected from N, O, and S, C₁-C₆ alkyl optionally substituted with one or more of halo, OR^{a} or NR^{a}R^{b}, and C₂-C₆ alkynyl optionally substituted with 4- to 12-membered heterocycloalkyl; wherein said C₃-C₈ cycloalkyl or 4- to 12-membered heterocycloalkyl are optionally substituted with one or more of halo, C(O)R^{a}, OR, NR^{a}R^{b}, 4- to 7-membered heterocycloalkyl, —C₁-C₆ alkylene-4- to 7-membered heterocycloalkyl, or C₁-C₄ alkyl optionally substituted with one or more of halo, OR^{a} or NR^{a}R^{b}, in which each of R^{a} and R^{b} independently is H or C₁-C₆ alkyl; or
R⁵ and one of R³ or R⁴ together with the atoms to which they are attached form phenyl or a 5- or 6-membered heteroaryl; or R⁵ and one of R^{3′} or R^{4′} together with the atoms to which they are attached form a 5- or 6-membered heteroaryl, in which the phenyl or 5- or 6-membered heteroaryl as formed is optionally substituted with one or more of halo, C₁-C₃ alkyl, hydroxyl or C₁-C₃ alkoxyl;
R⁶ is absent when X⁵ is N and ring A is a 6-membered heteroaryl; or R⁶ is -Q¹-T¹, in which Q¹ is a bond or C₁-C₆ alkylene, C₂-C₆ alkenylene, or C₂-C₆ alkynylene linker optionally substituted with one or more of halo, cyano, hydroxyl, oxo, or C₁-C₆ alkoxyl, and T¹ is H, halo, cyano, NR⁸R⁹, C(O)NR⁸R⁹, C(O)R⁹, OR⁸, OR⁹, or R^{S1}, in which R^{S1} is C₃-C₈ cycloalkyl, phenyl, 4- to 12-membered heterocycloalkyl containing 1-4 heteroatoms selected from N, O, and S, or a 5- or 6-membered heteroaryl and R^{S1} is optionally substituted with one or more of halo, C₁-C₆ alkyl, hydroxyl, oxo, —C(O)R⁹, —SO₂R⁸, —SO₂N(R⁸)₂, —NR⁸C(O)R⁹, NR⁸R⁹, or C₁-C₆ alkoxyl; and R⁶ is not NR⁸C(O)NR¹²R¹³; or
R⁶ and one of R² or R³ together with the atoms to which they are attached form phenyl or a 5- or 6-membered heteroaryl; or R⁶ and one of R^{2′} or R^{3′} together with the atoms to which they are attached form a 5- or 6-membered heteroaryl, in which the phenyl or 5- or 6-membered heteroaryl as formed is optionally substituted with one or more of halo, C₁-C₃ alkyl, hydroxyl, oxo (═O), C₁-C₃ alkoxyl, or -Q¹-T¹;
each R⁷ is independently oxo (═O) or -Q²-T², in which each Q² independently is a bond or C₁-C₆ alkylene, C₂-C₆ alkenylene, or C₂-C₆ alkynylene linker optionally substituted with one or more of halo, cyano, hydroxyl, amino, mono- or di-alkylamino, or C₁-C₆ alkoxyl, and each T² independently is H, halo, cyano, OR¹⁰, OR¹¹, C(O)R¹¹, NR¹⁰R¹¹, C(O)NR¹⁰R¹¹, NR¹⁰C(O)R¹¹, 5- to 10-membered heteroaryl, C₃-C₈ cycloalkyl, or 4- to 12-membered heterocycloalkyl containing 1-4 heteroatoms selected from N, O, and S, and wherein the 5- to 10-membered heteroaryl, C₃-C₈ cycloalkyl or 4- to 12-membered heterocycloalkyl is optionally substituted with one or more of halo, C₁-C₆ alkyl optionally substituted with NR^{x}R^{y}, hydroxyl, oxo, N(R⁸)₂, cyano, C₁-C₆ haloalkyl, —SO₂R⁸, or C₁-C₆ alkoxyl, each of R^{x} and R^{y} independently being H or C₁-C₆ alkyl; and R⁷ is not H or C(O)OR^{g};
each R⁸ independently is H or C₁-C₆ alkyl;
each R⁹ is independently -Q³-T³, in which Q³ is a bond or C₁-C₆ alkylene, C₂-C₆ alkenylene, or C₂-C₆ alkynylene linker optionally substituted with one or more of halo, cyano, hydroxyl, or C₁-C₆ alkoxyl, and T³ is H, halo, OR¹², OR¹³, NR¹²R¹³, NR¹²C(O)R¹³, C(O)NR¹²R¹³, C(O)R¹³, S(O)₂R¹³, S(O)₂NR¹²R¹³, or R^{S2}, in which R^{S2} is C₃-C₈ cycloalkyl, C₆-C₁₀ aryl, 4- to 12-membered heterocycloalkyl containing 1-4 heteroatoms selected from N, O, and S, or a 5- to 10-membered heteroaryl, and R^{S2} is optionally substituted with one or more -Q⁴-T⁴, wherein each Q⁴ independently is a bond or C₁-C₃ alkylene, C₂-C₃ alkenylene, or C₂-C₃ alkynylene linker each optionally substituted with one or more of halo, cyano, hydroxyl, or C₁-C₆ alkoxy, and each T⁴ independently is selected from the group consisting of H, halo, cyano, C₁-C₆ alkyl, C₃-C₈ cycloalkyl, C₆-C₁₀ aryl, 4- to 7-membered heterocycloalkyl containing 1-4 heteroatoms selected from N, O, and S, 5- to 6-membered heteroaryl, OR^{c}, C(O)R^{c}, S(O)₂R^{c}, NR^{c}R^{d}, C(O)NR^{c}R^{d}, and NR^{c}C(O)R^{d}, each of R^{c} and R^{d} independently being H or C₁-C₆ alkyl; or -Q⁴-T⁴ is oxo; or
R⁸ and R⁹ taken together with the nitrogen atom to which they are attached form a 4- to 12-membered heterocycloalkyl containing 1-4 heteroatoms selected from N, O and S, which is optionally substituted with one or more of -Q⁵-T⁵, wherein each Q⁵ independently is a bond or C₁-C₃ alkylene, C₂-C₃ alkenylene, or C₂-C₃ alkynylene linker each optionally substituted with one or more of halo, cyano, hydroxyl, or C₁-C₆ alkoxy, and each T⁵ independently is selected from the group consisting of H, halo, cyano, C₁-C₆ alkyl, C₃-C₈ cycloalkyl, C₆-C₁₀ aryl, 4- to 7-membered heterocycloalkyl containing 1-4 heteroatoms selected from N, O, and S, 5- to 6-membered heteroaryl, OR^{e}, C(O)R^{e}, S(O)₂R^{e}, S(O)₂NR^{e}R^{f}, NR^{e}R^{f}, C(O)NR^{e}R^{f}, and NR^{e}C(O)R^{f}, each of R^{e} and R^{f} independently being H or C₁-C₆ alkyl; or -Q⁵-T⁵ is oxo;
R¹⁰ is selected from the group consisting of H and C₁-C₆ alkyl;
R¹¹ is -Q⁶-T⁶, in which Q⁶ is a bond or C₁-C₆ alkylene, C₂-C₆ alkenylene, or C₂-C₆ alkynylene linker optionally substituted with one or more of halo, cyano, hydroxyl, oxo, or C₁-C₆ alkoxyl, and T⁶ is H, halo, OR^{g}, NR^{g}R^{h}, NR^{g}C(O)R^{h}, C(O)NR^{g}R^{h}, C(O)R^{g}, S(O)₂R^{g}, or R^{S3}, in which each of R^{g} and R^{h} independently is H, phenyl, C₃-C₈ cycloalkyl, or C₁-C₆ alkyl optionally substituted with C₃-C₈ cycloalkyl, or R^{g} and R^{h} together with the nitrogen atom to which they are attached form a 4- to 12-membered heterocycloalkyl containing 1-4 heteroatoms selected from N, O, and S, and R^{S3} is C₃-C₈ cycloalkyl, C₆-C₁₀ aryl, 4- to 12-membered heterocycloalkyl containing 1-4 heteroatoms selected from N, O and S, or a 5- to 10-membered heteroaryl, and R^{S3} is optionally substituted with one or more -Q⁷-T⁷, wherein each Q⁷ independently is a bond or C₁-C₃ alkylene, C₂-C₃ alkenylene, or C₂-C₃ alkynylene linker each optionally substituted with one or more of halo, cyano, hydroxyl, or C₁-C₆ alkoxy, and each T⁷ independently is selected from the group consisting of H, halo, cyano, C₁-C₆ alkyl, C₃-C₈ cycloalkyl, C₆-C₁₀ aryl, 4- to 7-membered heterocycloalkyl containing 1-4 heteroatoms selected from N, O, and S, 5- to 6-membered heteroaryl, OR^{j}, C(O)R^{j}, NR^{j}R^{k}, C(O)NR^{j}R^{k}, S(O)₂R^{j}, and NR^{j}C(O)R^{k}, each of R^{j} and R^{k} independently being H or C₁-C₆ alkyl optionally substituted with one or more halo; or -Q⁷-T⁷ is oxo; or
R¹⁰ and R¹¹ taken together with the nitrogen atom to which they are attached form a 4- to 12-membered heterocycloalkyl containing 1-4 heteroatoms selected from N, O, and S, which is optionally substituted with one or more of halo, C₁-C₆ alkyl, hydroxyl, or C₁-C₆ alkoxyl;
R¹² is H or C₁-C₆ alkyl;
R¹³ is C₁-C₆ alkyl, C₃-C₈ cycloalkyl, C₆-C₁₀ aryl, 4- to 12-membered heterocycloalkyl containing 1-4 heteroatoms selected from N, O, and S, or a 5- to 10-membered heteroaryl, each of which is optionally substituted with one or more -Q⁸-T⁸, wherein each Q⁸ independently is a bond or C₁-C₃ alkylene, C₂-C₃ alkenylene, or C₂-C₃ alkynylene linker each optionally substituted with one or more of halo, cyano, hydroxyl, or C₁-C₆ alkoxy, and each T⁸ independently is selected from the group consisting of H, halo, cyano, C₁-C₆ alkyl, C₃-C₈ cycloalkyl, C₆-C₁₀ aryl, 4- to 7-membered heterocycloalkyl containing 1-4 heteroatoms selected from N, O, and S, and 5- to 6-membered heteroaryl; or -Q⁸-T⁸ is oxo; and
n is 0, 1, 2, 3, or 4, provided that
the compound of Formula (I) is not
2-cyclohexyl-6-methoxy-N-[1-(1-methylethyl)-4-piperidinyl]-7-[3-(1-pyrrolidinyl)propoxy]-4-quinazolinamine;
N-(1-isopropylpiperidin-4-yl)-6-methoxy-2-(4-methyl-1,4-diazepan-1-yl)-7-(3-(piperidin-1-yl)propoxy)quinazolin-4-amine;
2-(4,4-difluoropiperidin-1-yl)-N-(1-isopropylpiperidin-4-yl)-6-methoxy-7-(3-(pyrrolidin-1-yl)propoxy)quinazolin-4-amine; or
2-(4-isopropyl-1,4-diazepan-1-yl)-N-(1-isopropylpiperidin-4-yl)-6-methoxy-7-(3-(piperidin-1-yl)propoxy)quinazolin-4-amine.

4-9. (canceled)

10. The method of claim 3, wherein the EHMT2 inhibitor is a compound of Formula (II): 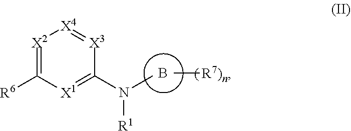
wherein
ring B is phenyl or pyridyl,
one or both of X¹ and X² are N while X³ is CR⁴ and X⁴ is CR⁵ or one or both of X¹ and X³ are N while X² is CR³ and X⁴ is CR⁵; and
n is 1, 2, or 3.

11. The method of claim 10, wherein the EHMT2 inhibitor is a compound of Formula (IIa1), (IIa2), (IIa3), (IIa4), or (IIa5):

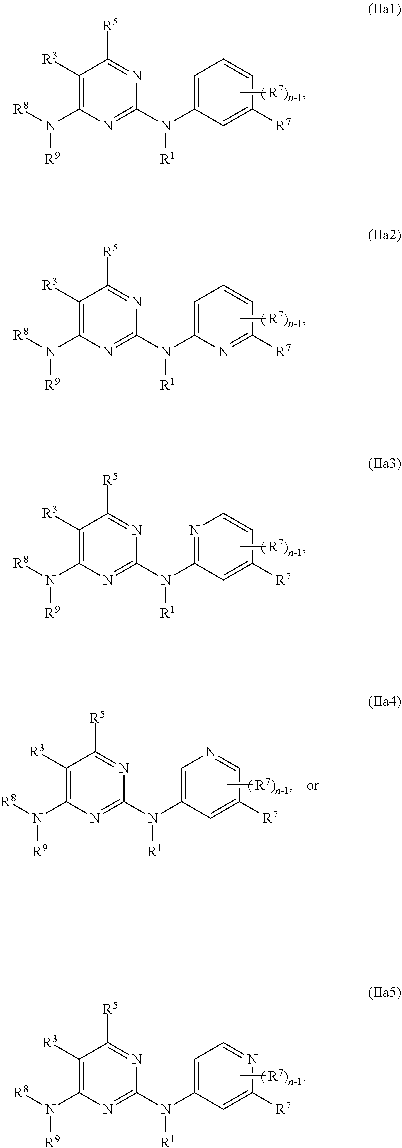

12-19. (canceled)

20. The method of claim 3, wherein the EHMT2 inhibitor is a compound of Formula (III): 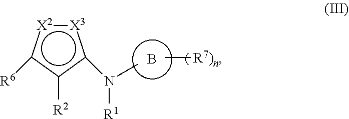
wherein
ring B is phenyl or pyridyl,
at least one of X² and X³ is N; and
n is 1 or 2.

21-25. (canceled)

26. The method of claim 3, wherein the EHMT2 inhibitor is a compound of Formula (IV): 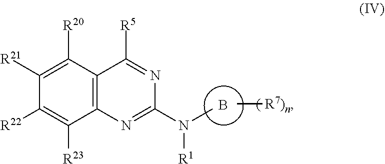
wherein
ring B is C₃-C₆ cycloalkyl;
each of R²⁰, R²¹, R²² and R²³ independently is H, halo, C₁-C₃ alkyl, hydroxyl, or C₁-C₃ alkoxyl; and
n is 1 or 2.

27-45. (canceled)

46. The method of claim 1, wherein the EHMT2 inhibitor is a compound of Formula (V): 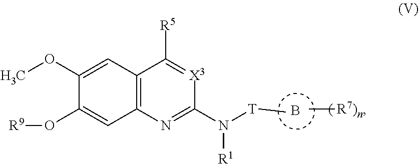
wherein
ring B is absent or C₃-C₆ cycloalkyl;
X³ is N or CR⁴ in which R⁴ is H or C₁-C₄ alkyl;
R¹ is H or C₁-C₄ alkyl;
or when B is absent, T and R¹ together with the atoms to which they are attached optionally form a 4-7 membered heterocycloalkyl or 5-6 membered heteroaryl, each of which is optionally substituted with (R⁷)ₙ; or when B is absent, T is H and n is 0;
each R⁷ is independently oxo (═O) or -Q²-T², in which each Q² independently is a bond or C₁-C₆ alkylene, C₂-C₆ alkenylene, or C₂-C₆ alkynylene linker optionally substituted with one or more of halo, cyano, hydroxyl, amino, mono- or di-alkylamino, or C₁-C₆ alkoxyl, and each T² independently is H, halo, OR¹⁰, OR¹¹, C(O)R¹¹, NR¹⁰R¹¹, C(O)NR¹⁰R¹¹, NR¹⁰C(O)R¹¹, C₃-C₈ cycloalkyl, or 4- to 12-membered heterocycloalkyl containing 1-4 heteroatoms selected from N, O, and S, and wherein the C₃-C₈ cycloalkyl or 4- to 12-membered heterocycloalkyl is optionally substituted with one or more of halo, C₁-C₆ alkyl optionally substituted with NR^{x}R^{y}, hydroxyl, oxo, N(R⁸)₂, cyano, C₁-C₆ haloalkyl, —SO₂R, or C₁-C₆ alkoxyl, each of R^{x} and R^{y} independently being H or C₁-C₆ alkyl; and R₇ is not H or C(O)OR^{g};
R⁵ is selected from the group consisting of C₁-C₆ alkyl, C₃-C₈ cycloalkyl and 4- to 12-membered heterocycloalkyl containing 1-4 heteroatoms selected from N, O and S, wherein the C₃-C₈ cycloalkyl and 4- to 12-membered heterocycloalkyl is optionally substituted with one or more of 4- to 7-membered heterocycloalkyl, —C₁-C₆ alkylene-4- to 7-membered heterocycloalkyl, —C(O)C₁-C₆ alkyl or C₁-C₆ alkyl optionally substituted with one or more of halo or OR^{a};
R⁹ is -Q³-T³, in which Q³ is a bond or C₁-C₆ alkylene, C₂-C₆ alkenylene, or C₂-C₆ alkynylene linker optionally substituted with one or more of halo, cyano, hydroxyl, or C₁-C₆ alkoxyl, and T³ is 4- to 12-membered heterocycloalkyl containing 1-4 heteroatoms selected from N, O, and S, optionally substituted with one or more -Q⁴-T⁴, wherein each Q⁴ independently is a bond or C₁-C₃ alkylene, C₂-C₃ alkenylene, or C₂-C₃ alkynylene linker each optionally substituted with one or more of halo, cyano, hydroxyl, or C₁-C₆ alkoxy, and each T⁴ independently is selected from the group consisting of H, halo, cyano, C₁-C₆ alkyl, C₃-C₈ cycloalkyl, C₆-C₁₀ aryl, 4- to 7-membered heterocycloalkyl containing 1-4 heteroatoms selected from N, O, and S, 5- to 6-membered heteroaryl, OR^{c}, C(O)R^{c}, S(O)₂R^{c}, NR^{c}R^{d}, C(O)NR^{c}R^{d}, and NR^{c}C(O)R^{d}, each of R^{c} and R^{d} independently being H or C₁-C₆ alkyl; or -Q⁴-T⁴ is oxo; and
n is 0, 1 or 2.

47-48. (canceled)

49. The method of claim 3, wherein the EHMT2 inhibitor is a compound of Formula (VII): 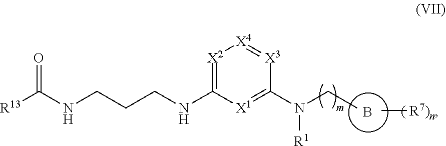
wherein m is 1 or 2 and n is 0, 1, or 2.

50. (canceled)

51. The method of claim 3, wherein the EHMT2 inhibitor is a compound of Formula (VIIIa): 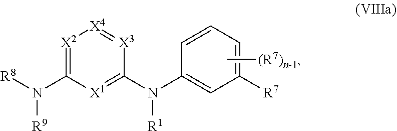
wherein
X¹ is Nor CR²;
X² is N or CR³;
X³ is N or CR⁴;
X⁴ is N or CR⁵;
R² is selected from the group consisting of H, C₃-C₈ cycloalkyl, and C₁-C₆ alkyl optionally substituted with one or more of halo, ORV, or NR^{a}R^{b};
each of R³ and R⁴ is H; and
R⁵ are independently selected from the group consisting of H, C₃-C₈ cycloalkyl, and C₁-C₆ alkyl optionally substituted with one or more of halo or OR^{a}; or
R⁵ and one of R³ or R⁴ together with the atoms to which they are attached form phenyl or a 5- or 6-membered heteroaryl; and
wherein at least one of R² or R⁵ is not H.

52. The method of claim 3, wherein the EHMT2 inhibitor is a compound of Formula (VIIIb): 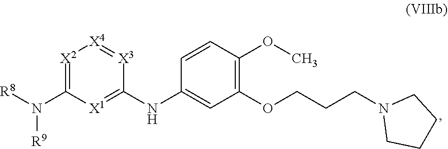
wherein
X¹ is N or CR²;
X² is N or CR³;
X³ is N or CR⁴;
X⁴ is N or CR⁵;
R² is selected from the group consisting of H, C₃-C₈ cycloalkyl, and C₁-C₆ alkyl each of R³ and R⁴ is H; and
R⁵ is selected from the group consisting of H, C₃-C₈ cycloalkyl, and C₁-C₆ alkyl; or
R⁵ and one of R³ or R⁴ together with the atoms to which they are attached form phenyl or a 5- or 6-membered heteroaryl; and
wherein at least one of R² or R⁵ is not H.

53. The method of claim 3, wherein the EHMT2 inhibitor is a compound of Formula (VIIIc): 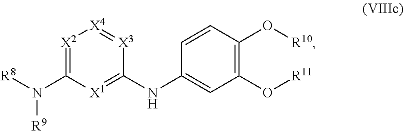
wherein
X¹ is Nor CR²;
X² is N or CR³;
X³ is N or CR⁴;
X⁴ is N or CR⁵;
R² is selected from the group consisting of H, C₃-C₈ cycloalkyl, and C₁-C₆ alkyl each of R³ and R⁴ is H; and
R⁵ is selected from the group consisting of H, C₃-C₈ cycloalkyl, and C₁-C₆ alkyl; or
R⁵ and one of R³ or R⁴ together with the atoms to which they are attached form phenyl or a 5- or 6-membered heteroaryl; and
wherein at least one of R² or R⁵ is not H.

54. The method of claim 1, wherein the EHMT2 inhibitor is a compound of (IX): 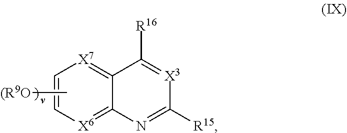
or a tautomer thereof, or a pharmaceutically acceptable salt of the compound or the tautomer, wherein
X⁶ is N or CH;
X⁷ is N or CH;
X³ is N or CR⁴;
R⁴ is selected from the group consisting of H, halo, cyano, C₁-C₆ alkoxyl, C₆-C₁₀ aryl, NR^{a}R^{b}, C(O)NR^{a}R^{b}, NR^{a}C(O)R^{b}, C₃-C₈ cycloalkyl, 4- to 7-membered heterocycloalkyl, 5- to 6-membered heteroaryl, and C₁-C₆ alkyl, wherein C₁-C₆ alkoxyl and C₁-C₆ alkyl are optionally substituted with one or more of halo, OR^{a}, or NR^{a}R^{b}, in which each of R^{a} and R^{b} independently is H or C₁-C₆ alkyl;
each R⁹ is independently -Q³-T³, in which each Q³ is independently a bond or C₁-C₆ alkylene, C₂-C₆ alkenylene, or C₂-C₆ alkynylene linker optionally substituted with one or more of halo, cyano, hydroxyl, or C₁-C₆ alkoxyl, and each T³ is independently H, halo, OR¹², OR¹³, NR¹²R¹³, NR¹²C(O)R¹³, C(O)NR¹²R¹³, C(O)R¹³, S(O)₂R¹³, S(O)₂NR¹²R¹³, or R^{S2}, in which each R^{S2} is independently C₃-C₈ cycloalkyl, C₆-C₁₀ aryl, 4- to 12-membered heterocycloalkyl containing 1-4 heteroatoms selected from N, O, and S, or a 5- to 10-membered heteroaryl, and each R^{S2} is independently optionally substituted with one or more -Q⁴-T⁴, wherein each Q⁴ independently is a bond or C₁-C₃ alkylene, C₂-C₃ alkenylene, or C₂-C₃ alkynylene linker each optionally substituted with one or more of halo, cyano, hydroxyl, or C₁-C₆ alkoxy, and each T⁴ independently is selected from the group consisting of H, halo, cyano, C₁-C₆ alkyl, C₃-C₈ cycloalkyl, C₆-C₁₀ aryl, 4- to 7-membered heterocycloalkyl containing 1-4 heteroatoms selected from N, O, and S, 5- to 6-membered heteroaryl, OR^{c}, C(O)R^{c}, S(O)₂R^{c}, N^{c}R^{d}, C(O)NR^{c}R^{d}, and NR^{c}C(O)R^{d}, each of R^{c} and R^{d} independently being H or C₁-C₆ alkyl; or -Q⁴-T⁴ is oxo; or
R¹² is H or C₁-C₆ alkyl;
R¹³ is C₁-C₆ alkyl, C₃-C₈ cycloalkyl, C₆-C₁₀ aryl, 4- to 12-membered heterocycloalkyl containing 1-4 heteroatoms selected from N, O, and S, or a 5- to 10-membered heteroaryl, each of which is optionally substituted with one or more -Q⁸-T⁸, wherein each Q⁸ independently is a bond or C₁-C₃ alkylene, C₂-C₃ alkenylene, or C₂-C₃ alkynylene linker each optionally substituted with one or more of halo, cyano, hydroxyl, or C₁-C₆ alkoxy, and each T⁸ independently is selected from the group consisting of H, halo, cyano, C₁-C₆ alkyl, C₃-C₈ cycloalkyl, C₆-C₁₀ aryl, 4- to 7-membered heterocycloalkyl containing 1-4 heteroatoms selected from N, O, and S, and 5- to 6-membered heteroaryl; or -Q⁹-T⁹ is oxo;
R¹⁶ is C₁-C₆ alkyl, NHR¹⁷, C₃-C₈ cycloalkyl, C₆-C₁₀ aryl, 4- to 12-membered heterocycloalkyl containing 1-4 heteroatoms selected from N, O, and S, or 5- to 10-membered heteroaryl, wherein each of said C₁-C₆ alkyl, C₃-C₈ cycloalkyl, C₆-C₁₀ aryl, 4- to 12-membered heterocycloalkyl, and 5- to 10-membered heteroaryl is optionally substituted with one or more -Q⁹-T⁹, wherein each Q⁹ independently is a bond or C₁-C₃ alkylene, C₂-C₃ alkenylene, or C₂-C₃ alkynylene linker each optionally substituted with one or more of halo, cyano, hydroxyl, or C₁-C₆ alkoxy, and each T⁹ independently is selected from the group consisting of H, halo, cyano, C₁-C₆ alkyl, C₃-C₈ cycloalkyl, C₆-C₁₀ aryl, 4- to 7-membered heterocycloalkyl containing 1-4 heteroatoms selected from N, O, and S, and 5- to 6-membered heteroaryl; or -Q⁹-T⁹ is oxo;
R¹⁶ is C₁-C₆ alkyl, C₂-C₆ alkenyl, C₂-C₆ alkynyl, C₃-C₈ cycloalkyl, C₆-C₁₀ aryl, 4- to 12-membered heterocycloalkyl containing 1-4 heteroatoms selected from N, O, and S, or a 5- to 10-membered heteroaryl, each of which is optionally substituted with one or more -Q¹⁰-T¹⁰, wherein each Q¹⁰ independently is a bond or C₁-C₃ alkylene, C₂-C₃ alkenylene, or C₂-C₃ alkynylene linker each optionally substituted with one or more of halo, cyano, hydroxyl, or C₁-C₆ alkoxy, and each T¹⁰ independently is selected from the group consisting of H, halo, cyano, C₁-C₆ alkyl, C₃-C₈ cycloalkyl, C₆-C₁₀ aryl, 4- to 7-membered heterocycloalkyl containing 1-4 heteroatoms selected from N, O, and S, and 5- to 6-membered heteroaryl; or -Q¹⁰-T¹⁰ is oxo;
R¹⁷ is H or C₁-C₆ alkyl; and
v is 0, 1, or 2.

55-66. (canceled)

67. The method of claim 1, wherein the EHMT2 inhibitor is a compound of Formula (I′): 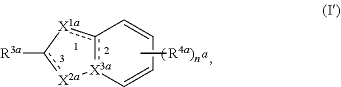
or a tautomer thereof, or a pharmaceutically acceptable salt of the compound or the tautomer, wherein
X^{1a} is O, S, CR^{1a}R^{11a}, or NR^{1a′} when [symbol] is a single bond, or X^{1a} is N when [symbol] is a double bond;
X^{2a} is N or CR^{2a} when [symbol] is a double bond, or X^{2a} is NR^{2a′} when [symbol] is a single bond;
X^{3a} is N or C; when X^{3a} is N, [symbol] is a double bond and [symbol] is a single bond, and when X^{3a} is C, [symbol] is a single bond and [symbol] is a double bond;
each of R^{1a}, R^{2a} and R^{11a}, independently, is -Q^{1a}-T^{1a}, in which each Q^{1a} independently is a bond or C₁-C₆ alkylene, C₂-C₆ alkenylene, or C₂-C₆ alkynylene linker optionally substituted with one or more of halo, cyano, hydroxyl, or C₁-C₆ alkoxyl, and each T^{1a} independently is H, halo, cyano, NR^{5a}R^{6a},l C(O)NR^{5a}R^{6a}, —OC(O)NR^{5a}R^{6a}, C(O)OR^{5a}, —OC(O)R^{5a}, C(O)R^{5a}, —NR^{5a}C(O)R^{6a}, —NR^{5a}C(O)OR^{6a}, OR^{5a}, or R^{S1a}, in which R^{S1a} is C₃-C₁₂ cycloalkyl, phenyl, 4- to 12-membered heterocycloalkyl containing 1-4 heteroatoms selected from N, O, and S, or a 5- or 6-membered heteroaryl and R^{S1a} is optionally substituted with one or more of halo, C₁-C₆ alkyl, hydroxyl, oxo, —C(O)R^{6a}, —SO₂R^{5a}, —SO₂N(R^{5a})₂, —NR^{5a}C(O)R^{6a}, amino, mono- or di-alkylamino, or C₁-C₆ alkoxyl; or
R^{1a} and R^{11a} together with the carbon atom to which they are attached form a C₃-C₁₂ cycloalkyl or 4- to 12-membered heterocycloalkyl containing 1-4 heteroatoms selected from N, O, and S, wherein the C₃-C₁₂ cycloalkyl or 4- to 12-membered heterocycloalkyl is optionally substituted with one or more of halo, C₁-C₆ alkyl, hydroxyl, oxo, amino, mono- or di-alkylamino, or C₁-C₆ alkoxyl;
each of R^{1a′} and R^{2a′}, independently, is -Q^{2a}-T^{2a}, in which each Q^{2a} is independently a bond or C₁-C₆ alkylene, C₂-C₆ alkenylene, or C₂-C₆ alkynylene linker optionally substituted with one or more of halo, cyano, hydroxyl, or C₁-C₆ alkoxyl, and each T^{2a} is independently H, halo, cyano, or R^{S2a}, in which each R^{S2a} is independently C₃-C₁₂ cycloalkyl, phenyl, 4- to 12-membered heterocycloalkyl containing 1-4 heteroatoms selected from N, O, and S, or a 5- or 6-membered heteroaryl and each R^{S2a} is independently optionally substituted with one or more of halo, C₁-C₆ alkyl, hydroxyl, oxo, —C(O)R^{6a}, —SO₂R^{5a}, —SO₂N(R^{5a})₂, —NR^{5a}C(O)R^{6a}, amino, mono- or di-alkylamino, or C₁-C₆ alkoxyl;
R^{3a} is H, NR^{aa}R^{ba}, OR^{aa}, or R^{S4a}, in which R^{S4a} is C₁-C₆ alkyl, C₂-C₆ alkenyl, C₂-C₆ alkynyl, C₃-C₁₂ cycloalkyl, phenyl, 5- or 6-membered heteroaryl, or 4- to 12-membered heterocycloalkyl containing 1-4 heteroatoms selected from N, O, and S, wherein each of R^{aa} and R^{ba} independently is H or R^{S5a}, or R^{aa} and R^{ba} together with the nitrogen atom to which they are attached form a 4- to 12-membered heterocycloalkyl containing 1-4 heteroatoms selected from N, O, and S; in which each R^{S5a} is independently C₁-C₆ alkyl, phenyl, 5- or 6-membered heteroaryl, or 4- to 12-membered heterocycloalkyl containing 1-4 heteroatoms selected from N, O, and S, and each of R^{S4a}, R^{S5a}, and the heterocycloalkyl formed by R^{aa} and R^{ba} is independently optionally substituted with one or more of halo, hydroxyl, oxo, CN, amino, mono- or di-alkylamino, C₁-C₆ alkyl, C₁-C₆ alkoxyl, C₃-C₁₂ cycloalkyl, phenyl, 5- or 6-membered heteroaryl, or 4- to 12-membered heterocycloalkyl containing 1-4 heteroatoms selected from N, O, and S, or alternatively;
R^{3a} and one of R^{1a′}, R^{2a′}, R^{1a}, R^{2a} and R^{11a}, together with the atoms to which they are attached, form a 5- or 6-membered heteroaryl that is optionally substituted with one or more of halo, C₁-C₃ alkyl, hydroxyl or C₁-C₃ alkoxyl; or
R^{3a} is oxo and [symbol] is a single bond;
each R^{4a} independently is -Q^{3a}-T^{3a}, in which each Q^{3a} independently is a bond or C₁-C₆ alkylene, C₂-C₆ alkenylene, or C₂-C₆ alkynylene linker optionally substituted with one or more of halo, cyano, hydroxyl, amino, mono- or di-alkylamino, or C₁-C₆ alkoxyl, and each T^{3a} independently is H, halo, cyano, OR^{7a}, OR^{8a}, C(O)R^{8a}, NR^{7a}R^{8a}, C(O)NR^{7a}R^{8a}, NR^{7a}C(O)R^{8a}, C₆-C₁₀ aryl, 5- to 10-membered heteroaryl, C₃-C₁₂ cycloalkyl, or 4- to 12-membered heterocycloalkyl containing 1-4 heteroatoms selected from N, O, and S, and wherein the C₆-C₁₀ aryl, 5- to 10-membered heteroaryl, C₃-C₁₂ cycloalkyl or 4- to 12-membered heterocycloalkyl is optionally substituted with one or more of halo, hydroxyl, cyano, C₁-C₆ haloalkyl, —SO₂R^{5a}, C₁-C₆ alkoxyl or C₁-C₆ alkyl optionally substituted with one or more of NR^{5a}R^{6a};
each of R^{5a}, R^{6a}, and R^{7a}, independently, is H or C₁-C₆ alkyl optionally substituted with one or more of halo, cyano, hydroxyl, amino, mono- or di-alkylamino, or C₁-C₆ alkoxyl;
R^{8a} is -Q^{4a}-T^{4a}, in which Q^{4a} is a bond or C₁-C₆ alkylene, C₂-C₆ alkenylene, or C₂-C₆ alkynylene linker optionally substituted with one or more of halo, cyano, hydroxyl, or C₁-C₆ alkoxyl, and T^{4a} is H, halo, or R^{S3a}, in which R^{S3a} is C₃-C₁₂ cycloalkyl, C₆-C₁₀ aryl, 4- to 12-membered heterocycloalkyl containing 1-4 heteroatoms selected from N, O and S, or a 5- to 10-membered heteroaryl, and R^{S3a} is optionally substituted with one or more -Q^{5a}-T^{5a}, wherein each Q^{5a} independently is a bond or C₁-C₃ alkylene, C₂-C₃ alkenylene, or C₂-C₃ alkynylene linker each optionally substituted with one or more of halo, cyano, hydroxyl, or C₁-C₆ alkoxy, and each T^{5a} independently is selected from the group consisting of H, halo, cyano, C₁-C₆ alkyl, C₃-C₁₂ cycloalkyl, C₆-C₁₀ aryl, 4- to 7-membered heterocycloalkyl containing 1-4 heteroatoms selected from N, O, and S, 5- to 6-membered heteroaryl, OR^{ca}, C(O)R^{ca}, NR^{ca}R^{da}, C(O)NR^{ca}R^{da}, S(O)₂R^{ca}, and NR^{ca}C(O)R^{da}, each of R^{ca} and R^{da} independently being H or C₁-C₆ alkyl optionally substituted with one or more halo; or -Q^{5a}-T^{5a} is oxo; and
n is 1, 2, 3, or 4.

68. The method of claim 1, wherein the EHMT2 inhibitor is a compound of Formula (I″), (II″), or (III″): 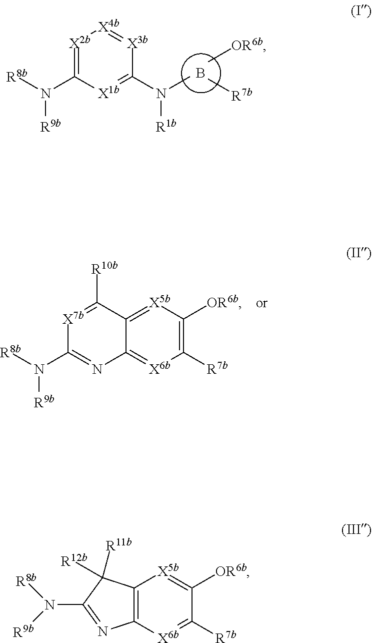
or a tautomer thereof, or a pharmaceutically acceptable salt of the compound or the tautomer, wherein
X^{1b} is N or CR^{2b};
X^{2b} is N or CR^{3b};
X^{3b} is N or CR^{4b};
X^{4b} is N or CR^{5b};
each of X^{5b}, X^{6b} and X^{7b} is independently N or CH;
B is C₆-C₁₀ aryl or 5- to 10-membered heteroaryl;
R^{1b} is H or C₁-C₄ alkyl;
each of R^{2b}, R^{3b}, R^{4b}, and R^{5b}, independently is selected from the group consisting of H, halo, cyano, C₁-C₆ alkoxyl, C₆-C₁₀ aryl, OH, NR^{ab}R^{bb}, C(O)NR^{ab}R^{bb}, NR^{ab}C(O)R^{bb}, C(O)OR^{ab}, OC(O)R^{ab}, OC(O)NR^{ab}R^{bb}, NR^{ab}C(O)OR^{bb}, C₃-C₈ cycloalkyl, 4- to 7-membered heterocycloalkyl, 5- to 6-membered heteroaryl, C₁-C₆ alkyl, C₂-C₆ alkenyl, and C₂-C₆ alkynyl, wherein the C₆-C₁₀ aryl, C₃-C₈ cycloalkyl, 4- to 7-membered heterocycloalkyl, 5- to 6-membered heteroaryl, C₁-C₆ alkoxyl, C₁-C₆ alkyl, C₂-C₆ alkenyl, and C₂-C₆ alkynyl, are each optionally substituted with one or more of halo, OR^{ab}, or NR^{ab}R^{bb}, in which each of R^{ab} and R^{bb} independently is H or C₁-C₆ alkyl;
R^{6b} is -Q^{1b}-T^{1b}, in which Q^{1b} is a bond, or C₁-C₆ alkylene, C₂-C₆ alkenylene, or C₂-C₆ alkynylene linker each optionally substituted with one or more of halo, cyano, hydroxyl, oxo, or C₁-C₆ alkoxyl, and T^{1b} is H, halo, cyano, or R^{S1b}, in which R^{S1b} is C₃-C₈ cycloalkyl, phenyl, 4- to 12-membered heterocycloalkyl containing 1-4 heteroatoms selected from N, O, and S, or a 5- or 6-membered heteroaryl and R^{S1b} is optionally substituted with one or more of halo, C₁-C₆ alkyl, C₂-C₆ alkenyl, C₂-C₆ alkynyl, hydroxyl, oxo, —C(O)R^{cb}, —C(O)OR^{cb}, —SO₂R^{cb}, —SO₂N(R^{cb})₂, —NR^{cb}C(O)R^{db}, —C(O)NR^{cb}R^{db}, —NR^{cb}C(O)OR^{db}, —OC(O)NR^{cb}R^{db}, NR^{cb}R^{db}, or C₁-C₆ alkoxyl, in which each of R^{cb} and R^{db} independently is H or C₁-C₆ alkyl;
R^{7b} is -Q^{2b}-T^{2b}, in which Q^{2b} is a bond, C(O)NR^{cb}, or NR^{cb}C(O), R^{cb} being H or C₁-C₆ alkyl and T^{2b} is 5- to 10-membered heteroaryl or 4- to 12-membered heterocycloalkyl, and wherein the 5- to 10-membered heteroaryl or 4- to 12-membered heterocycloalkyl is optionally substituted with one or more -Q^{3b}-T^{3b}, wherein each Q^{3b} independently is a bond or C₁-C₃ alkylene linker each optionally substituted with one or more of halo, cyano, hydroxyl, or C₁-C₆ alkoxy, and each T^{3b} independently is selected from the group consisting of H, halo, cyano, C₁-C₆ alkyl, C₂-C₆ alkenyl, C₂-C₆ alkynyl, C₃-C₈ cycloalkyl, C₆-C₁₀ aryl, 4- to 7-membered heterocycloalkyl containing 1-4 heteroatoms selected from N, O, and S, 5- to 6-membered heteroaryl, OR^{fb}, C(O)R^{fb}, C(O)OR^{fb}, OC(O)R^{fb}, S(O)₂R^{fb}, NR^{fb}R^{gb}, OC(O)NR^{fb}R^{gb}, NR^{fb}C(O)OR^{gb}, C(O)NR^{fb}R^{gb}, and NR^{fb}C(O)R^{gb}, each of R^{fb} and R^{gb} independently being H or C₁-C₆ alkyl, in which the C₃-C₈ cycloalkyl, C₆-C₁₀ aryl, 4- to 7-membered heterocycloalkyl or 5- to 6-membered heteroaryl is optionally substituted with one or more halo, cyano, hydroxyl, C₁-C₆ alkyl, C₂-C₆ alkenyl, C₂-C₆ alkynyl, or C₁-C₆ alkoxy; or Q^{3b}-T^{3b} is oxo;
R^{8b} is H or C₁-C₆ alkyl;
R^{9b} is -Q^{4b}-T^{4b}, in which Q^{4b} is a bond or C₁-C₆ alkylene, C₂-C₆ alkenylene, or C₂-C₆ alkynylene linker each optionally substituted with one or more of halo, cyano, hydroxyl, or C₁-C₆ alkoxyl, and T^{4b} is H, halo, OR^{hb}, NR^{hb}R^{ib}, NR^{hb}C(O)R^{ib}, C(O)NR^{hb}R^{ib}, C(O)R^{hb}, C(O)OR^{hb}, NR^{hb}C(O)OR^{ib}, OC(O)NR^{hb}R^{ib}, S(O)₂R^{hb}, S(O)₂NR^{hb}R^{ib}, or R^{S2b}, in which each of R^{hb} and R^{ib} independently is H or C₁-C₆ alkyl, and R^{S2b} is C₃-C₈ cycloalkyl, C₆-C₁₀ aryl, 4- to 12-membered heterocycloalkyl containing 1-4 heteroatoms selected from N, O, and S, or a 5- to 10-membered heteroaryl, and R^{S2b} is optionally substituted with one or more -Q^{5b}-T^{5b}, wherein each Q^{5b} independently is a bond or C₁-C₃ alkylene linker each optionally substituted with one or more of halo, cyano, hydroxyl, or C₁-C₆ alkoxy, and each T^{5b} independently is selected from the group consisting of H, halo, cyano, C₁-C₆ alkyl, C₂-C₆ alkenyl, C₂-C₆ alkynyl, C₃-C₈ cycloalkyl, C₆-C₁₀ aryl, 4- to 7-membered heterocycloalkyl containing 1-4 heteroatoms selected from N, O, and S, 5- to 6-membered heteroaryl, OR^{jb}, C(O)R^{jb}, C(O)OR^{jb}, OC(O)R^{jb}, S(O)₂R^{jb}, NR^{jb}R^{kb}, OC(O)NR^{jb}R^{kb}, NR^{jb}C(O)OR^{kb}, C(O)NR^{jb}R^{kb}, and NR^{jb}C(O)R^{kb}, each of R^{jb} and R^{kb} independently being H or C₁-C₆ alkyl; or -Q^{5b}-T^{5b}- is oxo;
R^{10b} is 4- to 12-membered heterocycloalkyl containing 1-4 heteroatoms selected from N, O, and S, which is optionally substituted with one or more halo, cyano, hydroxyl, oxo, amino, mono- or di-alkylamino, C₁-C₆ alkyl, C₂-C₆ alkenyl, C₂-C₆ alkynyl, or C₁-C₆ alkoxy; and
R^{11b} and R^{12b} together with the carbon atom to which they are attached form a C₃-C₁₂ cycloalkyl or 4- to 12-membered heterocycloalkyl containing 1-4 heteroatoms selected from N, O, and S, wherein the C₃-C₁₂ cycloalkyl or 4- to 12-membered heterocycloalkyl is optionally substituted with one or more of halo, C₁-C₆ alkyl, C₂-C₆ alkenyl, C₂-C₆ alkynyl, hydroxyl, oxo, amino, mono- or di-alkylamino, or C₁-C₆ alkoxyl.

69-122. (canceled)

123. The method of claim 1, wherein the EHMT2 inhibitor is a compound of Formula (I′″), (II′″), or (III′″): 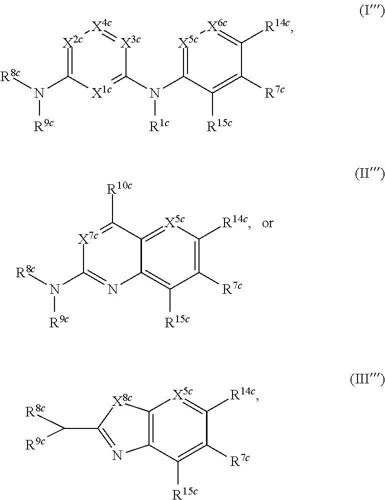
tautomers thereof, and pharmaceutically acceptable salts of the compounds and the tautomers, wherein
X^{1c} is N or CR^{2c};
X^{2c} is N or CR^{3c};
X^{3c} is N or CR^{4c};
X^{4c} is N or CR^{5c};
each of X^{5c}, X^{6c} and X^{7c} is independently N or CH;
X^{8c} is NR^{13C} or CR^{11c}R^{12c};
R^{1c} is H or C₁-C₄ alkyl;
each of R^{2c}, R^{3c}, R^{4c}, and R^{5c}, independently is selected from the group consisting of H, halo, cyano, C₁-C₆ alkoxyl, C₆-C₁₀ aryl, OH, NR^{ac}R^{bc}, C(O)NR^{ac}R^{bc}, NR^{ac}C(O)R^{bc}, C(O)OR^{ac}, OC(O)R^{ac}, OC(O)NR^{ac}R^{bc}, NR^{ac}C(O)OR^{bc}, C₃-C₈ cycloalkyl, 4- to 7-membered heterocycloalkyl, 5- to 6-membered heteroaryl, C₁-C₆ alkyl, C₂-C₆ alkenyl, and C₂-C₆ alkynyl, wherein the C₆-C₁₀ aryl, C₃-C₈ cycloalkyl, 4- to 7-membered heterocycloalkyl, 5- to 6-membered heteroaryl, C₁-C₆ alkoxyl, C₁-C₆ alkyl, C₂-C₆ alkenyl, and C₂-C₆ alkynyl, are each optionally substituted with one or more of halo, OR^{ac}, or NR^{ac}R^{bc}, in which each of R^{ac} and R independently is H or C₁-C₆ alkyl;
R^{6c} is -Q^{1c}-T^{1c}, in which Q^{1c} is a bond, or C₁-C₆ alkylene, C₂-C₆ alkenylene, or C₂-C₆ alkynylene linker each optionally substituted with one or more of halo, cyano, hydroxyl, oxo, or C₁-C₆ alkoxyl, and T^{1c} is H, halo, cyano, or R^{S1c}, in which R^{S1c} is C₃-C₈ cycloalkyl, phenyl, 4- to 12-membered heterocycloalkyl containing 1-4 heteroatoms selected from N, O, and S, or a 5- or 6-membered heteroaryl and R^{S1c} is optionally substituted with one or more of halo, C₁-C₆ alkyl, C₂-C₆ alkenyl, C₂-C₆ alkynyl, hydroxyl, oxo, —C(O)R^{cc}, —C(O)OR^{cc}, —SO₂R^{cc}, —SO₂N(R^{cc})₂, —NR^{cc}C(O)R^{dc}, —C(O)NR^{cc}R^{dc}, —NR^{cc}C(O)OR^{dc}, —OC(O)NR^{cc}R^{dc}, NR^{cc}R^{dc}, or C₁-C₆ alkoxyl, in which each of R^{cc} and R^{dc} independently is H or C₁-C₆ alkyl;
R^{7c} is -Q^{2c}-T^{2c}, in which Q^{2c} is a bond, C₁-C₆ alkylene, C₂-C₆ alkenylene, or C₂-C₆ alkynylene linker optionally substituted with one or more of halo, cyano, hydroxyl, amino, mono- or di-alkylamino, and T^{2c} is H, halo, cyano, OR^{cc}, OR^{fc}, C(O)R^{fc}, NR^{cc}R^{fc}, C(O)NR^{cc}R^{fc}, NR^{cc}C(O)R^{fc}, C₆-C₁₀ aryl, 5- to 10-membered heteroaryl, C₃-C₁₂ cycloalkyl, or 4- to 12-membered heterocycloalkyl, and wherein the C₆-C₁₀ aryl, 5- to 10-membered heteroaryl, C₃-C₁₂ cycloalkyl, or 4- to 12-membered heterocycloalkyl is optionally substituted with one or more -Q^{3c}-T^{3c}, wherein each Q^{3c} independently is a bond or C₁-C₃ alkylene linker each optionally substituted with one or more of halo, cyano, hydroxyl, or C₁-C₆ alkoxy, and each T^{3c} independently is selected from the group consisting of H, halo, cyano, C₁-C₆ alkyl, C₂-C₆ alkenyl, C₂-C₆ alkynyl, C₃-C₈ cycloalkyl, C₆-C₁₀ aryl, 4- to 7-membered heterocycloalkyl containing 1-4 heteroatoms selected from N, O, and S, 5- to 6-membered heteroaryl, OR^{cc}, OR^{fc}, C(O)R^{fc}, C(O)OR^{fc}, OC(O)R_{fc}, S(O)₂R^{fc}, NR^{fc}R^{gc}, OC(O)NR^{fc}R^{gc}, NR^{fc}C(O)OR^{gc}, C(O)NR^{fc}R^{gc}, and NR^{fc}C(O)R^{gc}; or -Q^{3c}-T^{3c}, is oxo;
each R^{cc} independently is H or C₁-C₆ alkyl optionally substituted with one or more of halo, cyano, hydroxyl, amino, mono- or di-alkylamino, or C₁-C₆ alkoxyl;
each of R^{fc} and R^{gc}, independently, is -Q^{6c}-T⁶, in which each Q is independently a bond or C₁-C₆ alkylene, C₂-C₆ alkenylene, or C₂-C₆ alkynylene linker each optionally substituted with one or more of halo, cyano, hydroxyl, or C₁-C₆ alkoxyl, and each T⁶ is independently H, halo, OR^{m1c}, NR^{m1c}R^{m2c}, NR^{m1c}C(O)R^{m2c}, C(O)NR^{m1c}R^{m2c}, C(O)R^{m1c}, C(O)OR^{m1c}, NR^{m1c}C(O)OR^{m2c}, OC(O)NR^{m1c}R^{m2c}, S(O)₂R^{m1c}, S(O)₂NR^{m1c}R^{m2c}, or R^{S3c}, in which each of R^{m1c} and R^{m2c} independently is H, C₁-C₆ alkyl, or (C₁-C₆ alkyl)-R^{S3c}, and each R^{S3c} is independently C₃-C₈ cycloalkyl, C₆-C₁₀ aryl, 4- to 12-membered heterocycloalkyl containing 1-4 heteroatoms selected from N, O, and S, or a 5- to 10-membered heteroaryl, and each R^{S3c} is independently optionally substituted with one or more -Q^{7c}-T^{7c}, wherein each Q^{7c} independently is a bond or C₁-C₃ alkylene linker each optionally substituted with one or more of halo, cyano, hydroxyl, or C₁-C₆ alkoxy, and each T^{7c} independently is selected from the group consisting of H, halo, cyano, C₁-C₆ alkyl, C₂-C₆ alkenyl, C₂-C₆ alkynyl, C₃-C₈ cycloalkyl, C₆-C₁₀ aryl, 4- to 7-membered heterocycloalkyl containing 1-4 heteroatoms selected from N, O, and S, 5- to 6-membered heteroaryl, OR^{n1c}, C(O)R^{n1c}, C(O)OR^{n1c}, OC(O)R^{n1c}, S(O)₂R^{n1c}, NR^{n1c}R^{n2c}, OC(O)NR^{n1c}R^{n2c}, NR^{n1c}C(O)OR^{n2c}, C(O)NR^{n1c}R^{n2c}, and NR^{n1c}C(O)R^{n2c}, each of R^{n1c} and R^{n2c} independently being H or C₁-C₆ alkyl; or -Q^{7c}-T^{7c} is oxo;
R^{8c} is H or C₁-C₆ alkyl;
R^{5c} is -Q^{4c}-T^{4c}, in which Q^{4c} is a bond or C₁-C₆ alkylene, C₂-C₆ alkenylene, or C₂-C₆ alkynylene linker each optionally substituted with one or more of halo, cyano, hydroxyl, or C₁-C₆ alkoxyl, and T^{4c} is H, halo, OR^{hc}, NR^{hc}R^{ic}, NR^{hc}(O)R^{ic}, C(O)NR^{hc}R^{ic}, C(O)R^{hc}, C(O)OR^{hc}, NR^{hc}C(O)OR^{ic}, OC(O)NR^{hc}R^{ic}, S(O)₂R^{hc}, S(O)₂NR^{hc}R^{ic}, or R^{S2c}, in which each of R^{hc} and R^{ic} independently is H or C₁-C₆ alkyl, and R^{S2c} is C₃-C₈ cycloalkyl, C₆-C₁₀ aryl, 4- to 12-membered heterocycloalkyl containing 1-4 heteroatoms selected from N, O, and S, or a 5- to 10-membered heteroaryl, and R^{S2c} is optionally substituted with one or more -Q⁵-T⁵, wherein each Q⁵ independently is a bond or C₁-C₃ alkylene linker each optionally substituted with one or more of halo, cyano, hydroxyl, or C₁-C₆ alkoxy, and each T^{5c} independently is selected from the group consisting of H, halo, cyano, C₁-C₆ alkyl, C₂-C₆ alkenyl, C₂-C₆ alkynyl, C₃-C₈ cycloalkyl, C₆-C₁₀ aryl, 4- to 7-membered heterocycloalkyl containing 1-4 heteroatoms selected from N, O, and S, 5- to 6-membered heteroaryl, OR^{jc}, C(O)R^{jc}, C(O)OR^{jc}, OC(O)R^{jc}, S(O)₂R^{jc}, NR^{jc}R^{kc}, OC(O)NR^{jc}R^{kc}, NR^{jc}C(O)OR^{kc}, C(O)NR^{jc}R^{kc}, and NR^{jc}C(O)R^{kc}, each of R^{jc} and R^{kc} independently being H or C₁-C₆ alkyl; or -Q^{5c}-T^{5c} is oxo;
R^{10c} is halo, C₁-C₆ alkyl, C₂-C₆ alkenyl, C₂-C₆ alkynyl, C₃-C₈ cycloalkyl, or 4- to 12-membered heterocycloalkyl containing 1-4 heteroatoms selected from N, O, and S, wherein each of the C₁-C₆ alkyl, C₂-C₆ alkenyl, C₂-C₆ alkynyl, C₃-C₈ cycloalkyl, and 4- to 12-membered heterocycloalkyl is optionally substituted with one or more halo, cyano, hydroxyl, oxo, amino, mono- or di-alkylamino, C₁-C₆ alkyl, C₂-C₆ alkenyl, C₂-C₆ alkynyl, C₁-C₆ alkoxy, C(O)NR^{jc}R^{kc}, or NR^{jc}C(O)R^{kc};
R^{11c} and R^{12c} together with the carbon atom to which they are attached form a C₃-C₁₂ cycloalkyl or 4- to 12-membered heterocycloalkyl containing 1-4 heteroatoms selected from N, O, and S, wherein the C₃-C₁₂ cycloalkyl or 4- to 12-membered heterocycloalkyl is optionally substituted with one or more of halo, C₁-C₆ alkyl, C₂-C₆ alkenyl, C₂-C₆ alkynyl, hydroxyl, oxo, amino, mono- or di-alkylamino, or C₁-C₆ alkoxyl;
R^{13c} is H, C₁-C₆ alkyl, C₂-C₆ alkenyl, C₂-C₆ alkynyl, C₃-C₁₂ cycloalkyl, or 4- to 12-membered heterocycloalkyl containing 1-4 heteroatoms selected from N, O, and S; and
each of R^{14c} and R^{15c}, independently, is H, halo, cyano, C₁-C₆ alkyl optionally substituted with one or more of halo or cyano, C₂-C₆ alkenyl optionally substituted with one or more of halo or cyano, C₂-C₆ alkynyl optionally substituted with one or more of halo or cyano, C₃-C₈ cycloalkyl optionally substituted with one or more of halo or cyano, or —OR^{6c}.

124-147. (canceled)

148. The method of claim 1, wherein the EHMT2 inhibitor is selected from those in Tables 1A-1E, 2-4, 4A, and 5, and pharmaceutically acceptable salts thereof.

149. The method of claim 1, wherein the EHMT2 inhibitor is a compound selected from Compound Nos. A75, CA51, CA70, DIR, D2, D3, D4R, D5R, D6, and D7, tautomers thereof, pharmaceutically acceptable salts thereof, and pharmaceutically acceptable salts of the tautomers.

150-175. (canceled)

176. The method of claim 1, further comprising administering to the subject in need thereof a therapeutically effective amount of one or more additional therapeutic agent.

177-183. (canceled)

184. The method of claim 176, wherein the one or more additional therapeutic agent comprises a standard-of-care agent, a therapeutic agent for a blood disorder, a histone deacetylase (HDAC) inhibitor, a DNA methyltransferase (DNMT) inhibitor or a hypomethylating agent, a BCL11A inhibitor, a KLF inhibitor, a GATA inhibitor, a c-MYB inhibitor, a PRMT1 inhibitor, a PRMT5 inhibitor, a LSD inhibitor, a P-selectin inhibitor, an immunosuppressive agent, an anti-inflammatory agent, an antihistamine, an aromatic L-amino acid decarboxylase (AADC) or DOPA decarboxylase inhibitor, an immunomodulatory drug, an interleukin-1 beta inhibitor, a cell transplant or a cell population transplant, a clinical intervention associated with preparing a subject for a transplantation procedure, a gene or a protein that induces expression of a target gene or to provide and/or express a functional copy of a gene product in a target cell, or any combination thereof.

185-218. (canceled)
